SEQUENCE LISTING (66 sequences)

SEQ ID NO: 1 - protein, 43 aa, synthetic construct
  REGION 1..43: note = Description of Artificial Sequence: Synthetic polypeptide
  VARIANT 4..33: note = MOD_RES - Any amino acid
  VARIANT 4..33: note = MISC_FEATURE - This region may encompass 20-30 residues
  REGION 1..43: note = See specification as filed for detailed description of substitutions and preferred embodiments
GTFXXXXXXXXXXXXXXXXXXXXXXXXXXXXXXGPSSGAPPPS

SEQ ID NO: 2 - protein, 37 aa, synthetic construct
  REGION 1..37: note = Description of Artificial Sequence: Synthetic polypeptide
  VARIANT 4: note = MOD_RES - Thr or Ile
  VARIANT 7: note = MOD_RES - Tyr, Val or Leu
  VARIANT 9: note = MOD_RES - Ile or Ser
  VARIANT 10: note = MOD_RES - Tyr, Gln or Ala
  VARIANT 11: note = MOD_RES - Leu, Met or 2-Methyl-L-leucine
  VARIANT 12: note = MOD_RES - Asp or Glu
  VARIANT 13: note = MOD_RES - Lys, Gly or Glu
  VARIANT 16: note = MOD_RES - Ala or Val
  VARIANT 17: note = MOD_RES - 2-aminoisobutyric acid or Lys
  VARIANT 18: note = MOD_RES - Glu or Leu
  VARIANT 20: note = MOD_RES - Val or Ile
  VARIANT 21: note = MOD_RES - Asn, Ala or Glu
  VARIANT 24: note = MOD_RES - Leu or Val
  VARIANT 25: note = MOD_RES - Ala or Lys
  VARIANT 37: note = C-terminal amino acid, amino acid ester, or amino acid amide that is optionally substituted with from 1-2 modifying groups
  REGION 1..37: note = See specification as filed for detailed description of substitutions and preferred embodiments
GTFXSDXSXXXXXQAXXXFXXWLXXGGPSSGAPPPSX

SEQ ID NO: 3 - protein, 37 aa, synthetic construct; identical to SEQ ID NO: 2
  REGION 1..37: note = Description of Artificial Sequence: Synthetic polypeptide
  VARIANT 4: note = MOD_RES - Thr or Ile
  VARIANT 7: note = MOD_RES - Tyr, Val or Leu
  VARIANT 9: note = MOD_RES - Ile or Ser
  VARIANT 10: note = MOD_RES - Tyr, Gln or Ala
  VARIANT 11: note = MOD_RES - Leu, Met or 2-Methyl-L-leucine
  VARIANT 12: note = MOD_RES - Asp or Glu
  VARIANT 13: note = MOD_RES - Lys, Gly or Glu
  VARIANT 16: note = MOD_RES - Ala or Val
  SITE 17: note = MOD_RES - 2-aminoisobutyric acid
  VARIANT 18: note = MOD_RES - Glu or Leu
  VARIANT 20: note = MOD_RES - Val or Ile
  VARIANT 21: note = MOD_RES - Asn, Ala or Glu
  VARIANT 24: note = MOD_RES - Leu or Val
  VARIANT 25: note = MOD_RES - Ala or Lys
  VARIANT 37: note = C-terminal amino acid, amino acid ester, or amino acid amide that is optionally substituted with from 1-2 modifying groups
  REGION 1..37: note = See specification as filed for detailed description of substitutions and preferred embodiments

SEQ ID NO: 4 - protein, 37 aa, synthetic construct
  REGION 1..37: note = Description of Artificial Sequence: Synthetic polypeptide
  SITE 17: note = MOD_RES - 2-aminoisobutyric acid
  VARIANT 37: note = C-terminal amino acid, amino acid ester, or amino acid amide that is optionally substituted with from 1-2 modifying groups
GTFTSDYSIYLDKQAAXEFVNWLLAGGPSSGAPPPSX

SEQ ID NO: 5 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDKQAAXEFVNWLLAGGPSSGAPPPSK

SEQ ID NO: 6 - protein, 37 aa, synthetic construct; identical to SEQ ID NO: 5
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de

SEQ ID NO: 7 - protein, 37 aa, synthetic construct; identical to SEQ ID NO: 5
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-((S)-4-carboxy-4-palmitamidobutanoyl)-L-lysine

SEQ ID NO: 8 - protein, 37 aa, synthetic construct; identical to SEQ ID NO: 5
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-palmitoyl-L-lysinamide

SEQ ID NO: 9 - protein, 37 aa, synthetic construct; identical to SEQ ID NO: 5
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-palmitoyl-L-lysine

SEQ ID NO: 10 - protein, 37 aa, synthetic construct
  REGION 1..37: note = Description of Artificial Sequence: Synthetic polypeptide
  REGION 1..37: note = See specification as filed for detailed description of substitutions and preferred embodiments
  SITE 37: note = N6-[N-(1-oxohexadecyl)-L-gamma-glutamyl]-L-lysinami de
GTFTSDVSSYLEGQAAKEFIAWLVKGGPSSGAPPPSK

SEQ ID NO: 11 - protein, 38 aa, synthetic construct
  REGION 1..38: note = Description of Artificial Sequence: Synthetic polypeptide
  REGION 1..38: note = See specification as filed for detailed description of substitutions and preferred embodiments
  SITE 38: note = N6-[N-(1-oxohexadecyl)-L-gamma-glutamyl]-L-lysinami de
GTFISDYSIAMDKIRQQDFVNWLLAQRGPSSGAPPPSK

SEQ ID NO: 12 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDLSIYLDKQAAXEFVNWLLAGGPSSGAPPPSK

SEQ ID NO: 13 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDEQAAXEFVNWLLAGGPSSGAPPPSK

SEQ ID NO: 14 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDKQAVXEFVNWLLAGGPSSGAPPPSK

SEQ ID NO: 15 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDKQAAXLFVNWLLAGGPSSGAPPPSK

SEQ ID NO: 16 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDKQAAXEFINWLLAGGPSSGAPPPSK

SEQ ID NO: 17 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDKQAAXEFVEWLLAGGPSSGAPPPSK

SEQ ID NO: 18 - protein, 37 aa, synthetic construct
  REGION 1..37: note = Description of Artificial Sequence: Synthetic polypeptide
  SITE 17: note = MOD_RES - 2-aminoisobutyric acid
  SITE 37: note = Amidated residue
GTFTSDYSIYLDKQAVXEFINWLLAGGPSSGAPPPSK

SEQ ID NO: 19 - protein, 37 aa, synthetic construct
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de
GTFTSDYSIQMDKQAAXEFVNWLLAGGPSSGAPPPSK

SEQ ID NO: 20 - protein, 37 aa, synthetic construct
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de
GTFTSDYSIAMDKQAAXEFVNWLLAGGPSSGAPPPSK

SEQ ID NO: 21 - protein, 37 aa, synthetic construct
  REGION 1..37: note = Description of Artificial Sequence: Synthetic polypeptide
  SITE 11: note = MOD_RES - 2-Methyl-L-leucine
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de
GTFTSDYSIYXDKQAAXEFVNWLLAGGPSSGAPPPSK

SEQ ID NO: 22 - protein, 36 aa, synthetic construct
  REGION 1..36: note = Description of Artificial Sequence: Synthetic polypeptide
  SITE 17: note = MOD_RES - 2-aminoisobutyric acid
  SITE 36: note = Amidated residue
GTFTSDYSIYLDRQAAXEFVNWLLAGGPSSGAPPPS

SEQ ID NO: 23 - protein, 38 aa, synthetic construct
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de
EGTFTSDYSIYLDKQAVXEFVNWLLAGGPSSGAPPPSK

SEQ ID NO: 24 - protein, 38 aa, synthetic construct
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 22: note = N6-((S)-4-carboxy-4-palmitamidobutanoyl)-L-lysine
  SITE 38: note = Amidated residue
EGTFTSDYSIYLDKQAAXEFVKWLLAGGPSSGAPPPSK

SEQ ID NO: 25 - protein, 40 aa, synthetic construct
  REGION 1..40: note = Description of Artificial Sequence: Synthetic polypeptide
  SITE 18: note = MOD_RES - 2-aminoisobutyric acid
  REGION 1..40: note = See specification as filed for detailed description of substitutions and preferred embodiments
  SITE 30: note = N6-((S)-4-carboxy-4-palmitamidobutanoyl)-L-lysine
  SITE 40: note = Amidated residue
EGTFTSDYSIYLDKQAAXEFVNWLLAGRGKGPSSGAPPPS

SEQ ID NO: 26 - protein, 38 aa, synthetic construct
  REGION 1..38: note = Description of Artificial Sequence: Synthetic polypeptide
  REGION 1..38: note = See specification as filed for detailed description of substitutions and preferred embodiments
  SITE 18: note = N6-((S)-4-carboxy-4-palmitamidobutanoyl)-L-lysine
  SITE 38: note = Amidated residue
EGTFTSDYSIYLDKQAAKEFVNWLLAGGPSSGAPPPSK

SEQ ID NO: 27 - protein, 38 aa, synthetic construct
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 8: note = N6-((S)-4-carboxy-4-palmitamidobutanoyl)-L-lysine
  SITE 38: note = Amidated residue
EGTFTSDKSIYLDKQAAXEFVNWLLAGGPSSGAPPPSK

SEQ ID NO: 28 - protein, 37 aa, synthetic construct; identical to SEQ ID NO: 5
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-((S)-4-carboxy-4-palmitamidobutanoyl)-L-lysinami de

SEQ ID NO: 29 - protein, 37 aa, synthetic construct
  REGION 1..37: note = Description of Artificial Sequence: Synthetic polypeptide
  SITE 10: note = MOD_RES - (S)-2-amino-3-(4-hydroxyphenyl)-2-methylpropanoic acid
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de
GTFTSDYSIXLDKQAAXEFVNWLLAGGPSSGAPPPSK

SEQ ID NO: 30 - protein, 37 aa, synthetic construct; identical to SEQ ID NO: 14
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de

SEQ ID NO: 31 - protein, 37 aa, synthetic construct
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 21: note = N6-((S)-4-carboxy-4-palmitamidobutanoyl)-L-lysine
  SITE 37: note = Amidated residue
GTFTSDYSIYLDKQAAXEFVKWLLAGGPSSGAPPPSK

SEQ ID NO: 32 - protein, 39 aa, synthetic construct
  REGION 1..39: note = Description of Artificial Sequence: Synthetic polypeptide
  SITE 17: note = MOD_RES - 2-aminoisobutyric acid
  REGION 1..39: note = See specification as filed for detailed description of substitutions and preferred embodiments
  SITE 29: note = N6-((S)-4-carboxy-4-palmitamidobutanoyl)-L-lysine
  SITE 39: note = Amidated residue
GTFTSDYSIYLDKQAAXEFVNWLLAGRGKGPSSGAPPPS

SEQ ID NO: 33 - protein, 37 aa, synthetic construct
  REGION 1..37: note = Description of Artificial Sequence: Synthetic polypeptide
  REGION 1..37: note = See specification as filed for detailed description of substitutions and preferred embodiments
  SITE 17: note = N6-((S)-4-carboxy-4-palmitamidobutanoyl)-L-lysine
  SITE 37: note = Amidated residue
GTFTSDYSIYLDKQAAKEFVNWLLAGGPSSGAPPPSK

SEQ ID NO: 34 - protein, 37 aa, synthetic construct
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 7: note = N6-((S)-4-carboxy-4-palmitamidobutanoyl)-L-lysine
  SITE 37: note = Amidated residue
GTFTSDKSIYLDKQAAXEFVNWLLAGGPSSGAPPPSK

SEQ ID NO: 35 - protein, 40 aa, synthetic construct
  REGION 1..40: note = Description of Artificial Sequence: Synthetic polypeptide
  VARIANT 4: note = MOD_RES - Thr or Ile
  VARIANT 7: note = MOD_RES - Tyr, Val, Leu or lysine residue substituted with a modifying group
  VARIANT 9: note = MOD_RES - Ile or Ser
  VARIANT 10: note = MOD_RES - Tyr, (S)-2-amino-3-(4-hydroxyphenyl)-2-methylpropanoic acid, Gln, Ala or 2-aminoisobutyric acid
  VARIANT 11: note = MOD_RES - Leu, Met or 2-Methyl-L-leucine
  VARIANT 12: note = MOD_RES - Asp or Glu
  VARIANT 13: note = MOD_RES - Lys, Gly, Arg or Glu
  VARIANT 14: note = MOD_RES - Gln or Ile
  VARIANT 15: note = MOD_RES - Ala, His or Arg
  VARIANT 16: note = MOD_RES - Ala, Gln or Val
  VARIANT 17: note = MOD_RES - Ala, 2-aminoisobutyric acid, Lys, lysine residue substituted with a modifying group, or Gln
  VARIANT 18: note = MOD_RES - Ala, Asp, Glu, 2-aminoisobutyric acid or Leu
  VARIANT 19: note = MOD_RES - Phe or Ala
  VARIANT 20: note = MOD_RES - Val or Ile
  VARIANT 21: note = MOD_RES - Asn, Ala, Gln, lysine residue substituted with a modifying group, or Glu
  VARIANT 24: note = MOD_RES - Ile, Leu or Val
  VARIANT 25: note = MOD_RES - Ala, Lys or Ile
  VARIANT 26..29: note = MOD_RES - This region may encompass GRG-lysine residue substituted with a modifying group or QR or Q or G
  VARIANT 39: note = MOD_RES - Ser or absent
  VARIANT 40: note = C-terminal amino acid, amino acid ester, or amino acid amide that is optionally substituted with from 1-2 modifying groups
  REGION 1..40: note = See specification as filed for detailed description of substitutions and preferred embodiments
GTFXSDXSXXXXXXXXXXXXXWLXXXXXXGPSSGAPPPXX

SEQ ID NO: 36 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDYSIYLDKQAAXEFVNWLLAGGPSSGAPPPSK

SEQ ID NO: 37 - protein, 38 aa, synthetic construct; identical to SEQ ID NO: 36
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  VARIANT 38: note = N6-[N-(1-oxohexadecyl)-L-gamma-glutamyl]-L-lysinami de, N6-((S)-4-carboxy-4-palmitamidobutanoyl)-L-lysine, N6-palmitoyl-L-lysinamide, or N6-palmitoyl-L-lysine

SEQ ID NO: 38 - protein, 38 aa, synthetic construct; identical to SEQ ID NO: 36
  REGION 1..38: note = Description of Artificial Sequence: Synthetic polypeptide
  SITE 1: note = MOD_RES - D-Glu
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de

SEQ ID NO: 39 - protein, 38 aa, synthetic construct
  REGION 1..38: note = Description of Artificial Sequence: Synthetic polypeptide
  REGION 1..38: note = See specification as filed for detailed description of substitutions and preferred embodiments
  SITE 38: note = N6-[N-(1-oxohexadecyl)-L-gamma-glutamyl]-L-lysinami de
EGTFTSDVSSYLEGQAAKEFIAWLVKGGPSSGAPPPSK

SEQ ID NO: 40 - protein, 39 aa, synthetic construct
  REGION 1..39: note = Description of Artificial Sequence: Synthetic polypeptide
  REGION 1..39: note = See specification as filed for detailed description of substitutions and preferred embodiments
  SITE 39: note = N6-[N-(1-oxohexadecyl)-L-gamma-glutamyl]-L-lysinami de
EGTFISDYSIAMDKIRQQDFVNWLLAQRGPSSGAPPPSK

SEQ ID NO: 41 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDLSIYLDKQAAXEFVNWLLAGGPSSGAPPPSK

SEQ ID NO: 42 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDYSIYLDEQAAXEFVNWLLAGGPSSGAPPPSK

SEQ ID NO: 43 - protein, 38 aa, synthetic construct; identical to SEQ ID NO: 23
  REGION 1..38: note = Description of Artificial Sequence: Synthetic polypeptide
  SITE 18: note = MOD_RES - 2-aminoisobutyric acid
  SITE 38: note = Amidated residue

SEQ ID NO: 44 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDYSIYLDKQAAXLFVNWLLAGGPSSGAPPPSK

SEQ ID NO: 45 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDYSIYLDKQAAXEFINWLLAGGPSSGAPPPSK

SEQ ID NO: 46 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDYSIYLDKQAAXEFVEWLLAGGPSSGAPPPSK

SEQ ID NO: 47 - protein, 38 aa, synthetic construct
  REGION 1..38: note = Description of Artificial Sequence: Synthetic polypeptide
  SITE 18: note = MOD_RES - 2-aminoisobutyric acid
  SITE 38: note = Amidated residue
EGTFTSDYSIYLDKQAVXEFINWLLAGGPSSGAPPPSK

SEQ ID NO: 48 - protein, 38 aa, synthetic construct
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de
EGTFTSDYSIQMDKQAAXEFVNWLLAGGPSSGAPPPSK

SEQ ID NO: 49 - protein, 38 aa, synthetic construct
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de
EGTFTSDYSIAMDKQAAXEFVNWLLAGGPSSGAPPPSK

SEQ ID NO: 50 - protein, 38 aa, synthetic construct
  REGION 1..38: note = Description of Artificial Sequence: Synthetic polypeptide
  SITE 12: note = MOD_RES - 2-Methyl-L-leucine
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de
EGTFTSDYSIYXDKQAAXEFVNWLLAGGPSSGAPPPSK

SEQ ID NO: 51 - protein, 37 aa, synthetic construct
  REGION 1..37: note = Description of Artificial Sequence: Synthetic polypeptide
  SITE 18: note = MOD_RES - 2-aminoisobutyric acid
  SITE 37: note = Amidated residue
EGTFTSDYSIYLDRQAAXEFVNWLLAGGPSSGAPPPS

SEQ ID NO: 52 - protein, 38 aa, synthetic construct; identical to SEQ ID NO: 23
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de

SEQ ID NO: 53 - protein, 38 aa, synthetic construct; identical to SEQ ID NO: 24
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  VARIANT 22: note = Lysine residue substituted with a modifying group
  SITE 38: note = Amidated residue

SEQ ID NO: 54 - protein, 40 aa, synthetic construct; identical to SEQ ID NO: 25
  REGION 1..40: note = Description of Artificial Sequence: Synthetic polypeptide
  SITE 18: note = MOD_RES - 2-aminoisobutyric acid
  VARIANT 30: note = Lysine residue substituted with a modifying group
  SITE 40: note = Amidated residue
  REGION 1..40: note = See specification as filed for detailed description of substitutions and preferred embodiments

SEQ ID NO: 55 - protein, 38 aa, synthetic construct; identical to SEQ ID NO: 26
  REGION 1..38: note = Description of Artificial Sequence: Synthetic polypeptide
  REGION 1..38: note = See specification as filed for detailed description of substitutions and preferred embodiments
  VARIANT 18: note = Lysine residue substituted with a modifying group
  SITE 38: note = Amidated residue

SEQ ID NO: 56 - protein, 38 aa, synthetic construct; identical to SEQ ID NO: 27
  REGION 1..38: note = Description of Artificial Sequence: Synthetic polypeptide
  SITE 18: note = MOD_RES - 2-aminoisobutyric acid
  REGION 1..38: note = See specification as filed for detailed description of substitutions and preferred embodiments
  SITE 8: note = N6-((S)-4-carboxy-4-palmitamidobutanoyl)-L-lysine
  SITE 38: note = Amidated residue

SEQ ID NO: 57 - protein, 37 aa, synthetic construct; identical to SEQ ID NO: 5
  REGION 1..37: note = Description of Artificial Sequence: Synthetic polypeptide
  SITE 17: note = MOD_RES - 2-aminoisobutyric acid
  REGION 1..37: note = See specification as filed for detailed description of substitutions and preferred embodiments
  SITE 37: note = N6-[N-(1-oxohexadecyl)-L-gamma-glutamyl]-L-lysinami de

SEQ ID NO: 58 - protein, 38 aa, synthetic construct
  REGION 1..38: note = Description of Artificial Sequence: Synthetic polypeptide
  SITE 11: note = MOD_RES - (S)-2-amino-3-(4-hydroxyphenyl)-2-methylpropanoic acid
  SITE 18: note = MOD_RES - 2-aminoisobutyric acid
  REGION 1..38: note = See specification as filed for detailed description of substitutions and preferred embodiments
  SITE 38: note = N6-[N-(1-oxohexadecyl)-L-gamma-glutamyl]-L-lysinami de
EGTFTSDYSIXLDKQAAXEFVNWLLAGGPSSGAPPPSK

SEQ ID NO: 59 - protein, 37 aa, synthetic construct; identical to SEQ ID NO: 4
  SITE 17: note = 2-aminoisobutyric acid
  VARIANT 37: note = C-terminal amino acid, amino acid ester, or amino acid amide that is optionally substituted with from 1-2 modifying groups

SEQ ID NO: 60 - protein, 38 aa, synthetic construct; identical to SEQ ID NO: 36
  SITE 1: note = 2-((2-oxo-2-((2-(3-oxomorpholino)ethyl)amino)ethyl) thio)acetyl modified residue
  SITE 18: note = 2-aminoisobutyric acid
  SITE 38: note = N6-[N-(1-oxohexadecyl)-L-gamma-glutamyl]-L-lysinami de

SEQ ID NO: 61 - protein, 38 aa, synthetic construct; identical to SEQ ID NO: 36
  SITE 1: note = 2-((2-oxo-2-((2-(2-oxopiperidin-1-yl)ethyl)amino)et hyl)thio)acetyl modified residue
  SITE 18: note = 2-aminoisobutyric acid
  SITE 38: note = N6-[N-(1-oxohexadecyl)-L-gamma-glutamyl]-L-lysinami de

SEQ ID NO: 62 - protein, 38 aa, synthetic construct; identical to SEQ ID NO: 36
  SITE 1: note = (S)-2-((2-((2-(3-hydroxy-2-oxopyrrolidin-1-yl)ethyl )amino)-2-oxoethyl)thio)acetyl modified residue
  SITE 18: note = 2-aminoisobutyric acid
  SITE 38: note = N6-[N-(1-oxohexadecyl)-L-gamma-glutamyl]-L-lysinami de

SEQ ID NO: 63 - protein, 38 aa, synthetic construct; identical to SEQ ID NO: 36
  SITE 1: note = (S)-2-((2-((2-(3-methyl-2-oxopyrrolidin-1-yl)ethyl) amino)-2-oxoethyl)thio)acetyl modified residue
  SITE 18: note = 2-aminoisobutyric acid
  SITE 38: note = N6-[N-(1-oxohexadecyl)-L-gamma-glutamyl]-L-lysinami de

SEQ ID NO: 64 - protein, 38 aa, synthetic construct; identical to SEQ ID NO: 36
  SITE 1: note = (R)-2-((2-((2-(3-methyl-2-oxopyrrolidin-1-yl)ethyl) amino)-2-oxoethyl)thio)acetyl modified residue
  SITE 18: note = 2-aminoisobutyric acid
  SITE 38: note = N6-[N-(1-oxohexadecyl)-L-gamma-glutamyl]-L-lysinami de

SEQ ID NO: 65 - protein, 38 aa, synthetic construct; identical to SEQ ID NO: 36
  SITE 1: note = (S)-2-((2-((2-(3-methyl-2,5-dioxopyrrolidin-1-yl)et hyl)amino)-2-oxoethyl)thio)acetyl modified residue
  SITE 18: note = 2-aminoisobutyric acid
  SITE 38: note = N6-[N-(1-oxohexadecyl)-L-gamma-glutamyl]-L-lysinami de

SEQ ID NO: 66 - protein, 38 aa, synthetic construct; identical to SEQ ID NO: 36
  SITE 1: note = 2-((2-((2-((3S,4R)-3,4-dimethyl-2,5-dioxopyrrolidin -1-yl)ethyl)amino)-2-oxoethyl)thio)acetyl modified residue
  SITE 18: note = 2-aminoisobutyric acid
  SITE 38: note = N6-[N-(1-oxohexadecyl)-L-gamma-glutamyl]-L-lysinami de

(19) United States
(12) Patent Application Publication
(10) Pub. No.: US 2026/0176320 A1
(43) Pub. Date: Jun. 25, 2026

(54) MODULATORS OF G-PROTEIN COUPLED RECEPTORS

(71) Applicant: Carmot Therapeutics Inc., Berkeley, CA (US)

(72) Inventors: Johan Enquist, San Francisco, CA (US); Shyam Krishnan, Novato, CA (US); Suman Atwal, San Jose, CA (US); Daniel Erlanson, San Francisco, CA (US); Raymond V. Fucini, San Bruno, CA (US); Stig Hansen, Kensington, CA (US); Andrew Sawayama, San Francisco, CA (US); Steven Sethofer, Emeryville, CA (US)

(73) Assignee: Carmot Therapeutics Inc., Berkeley, CA (US)

(21) Appl. No.: 19/091,745

(22) Filed: Mar. 26, 2025

Related U.S. Application Data
(63) Continuation of application No. 18/087,063, filed on Dec. 22, 2022, now abandoned.
Application No. 18/087,063 is a continuation of application No. 17/040,653, now Pat. No. 11,535,660.
(60) Provisional application No. 62/647,604, filed on Mar. 23, 2018.

(51) Int. Cl. C07K 14/605; A61K 38/00; A61K 47/54

(52) U.S. Cl. CPC C07K 14/605; A61K 47/545; A61K 38/00

(57) ABSTRACT

This disclosure features chemical entities (e.g., a compound or a pharmaceutically acceptable salt and/or hydrate and/or prodrug of the compound) that modulate (e.g., agonize or partially agonize or antagonize) glucagon-like peptide-1 receptor (“GLP-1R”) and/or the gastric inhibitory polypeptide receptor (“GIPR”). The chemical entities are useful, e.g., for treating a subject (e.g., a human) having a disease, disorder, or condition in which modulation (e.g., agonism, partial agonism or antagonism) of GLP-1R and/or GIPR activities is beneficial for the treatment or prevention of the underlying pathology and/or symptoms and/or progression of the disease, disorder, or condition. In some embodiments, the modulation results in an enhancement of (e.g., an increase in) existing levels (e.g., normal or below normal levels) of GLP-1R and/or GIPR activity (e.g., signaling). In some embodiments, the chemical entities described herein further modulate (e.g., attenuate, uncouple) β-arrestin signaling relative to what is observed with the native ligand. This disclosure also features compositions as well as other methods of using and making the said chemical entities.

### PRIORITY CLAIM

This application is a continuation of U.S. application Ser. No. 18/087,063, filed on Dec. 22, 2022, which is a continuation of U.S. application Ser. No. 17/040,653, filed on Sep. 23, 2020, which is a National Stage application under 35 U.S.C. § 371 of International Application No. PCT/US2019/023726, having an International Filing Date of Mar. 22, 2019, which claims the benefit of U.S. Provisional Application Ser. No. 62/647,604, filed on Mar. 23, 2018. The disclosures of those applications are incorporated by reference herein in their entirety.

### SEQUENCE LISTING

This application contains a Sequence Listing that has been submitted electronically as an XML file named “124921_C1002_SL_rev.XML.” The XML file, created on Sep. 5, 2024, is 177,982 bytes in size. The material in the XML file is hereby incorporated by reference in its entirety

### TECHNICAL FIELD

This disclosure features chemical entities (e.g., a compound or a pharmaceutically acceptable salt and/or hydrate and/or prodrug of the compound) that modulate (e.g., agonize or partially agonize or antagonize) glucagon-like peptide-1 receptor (“GLP-1R”) and/or the gastric inhibitory polypeptide receptor (“GIPR”). The chemical entities are useful, e.g., for treating a subject (e.g., a human) having a disease, disorder, or condition in which modulation (e.g., agonism, partial agonism or antagonism) of GLP-1R and/or GIPR activities is beneficial for the treatment or prevention of the underlying pathology and/or symptoms and/or progression of the disease, disorder, or condition. In some embodiments, the modulation results in an enhancement of (e.g., an increase in) existing levels (e.g., normal or below normal levels) of GLP-1R and/or GIPR activity (e.g., signaling). In some embodiments, the chemical entities described herein further modulate (e.g., attenuate, uncouple) β-arrestin signaling relative to what is observed with the native ligand. This disclosure also features compositions as well as other methods of using and making the said chemical entities.

### BACKGROUND

Diabetes mellitus type 2 (type-2 diabetes) is characterized by high blood glucose and insulin resistance. Type 2 diabetes as well as conditions that are co-morbid or sequela with type-2 diabetes affect tens of millions of people in the United States alone. Type-2 diabetes is frequently associated with obesity.

Nonalcoholic steatohepatitis (NASH) is liver inflammation and damage caused by a buildup of fat in the liver. It is part of a group of conditions called nonalcoholic fatty liver disease (NAFLD). NASH and NAFLD tend to develop in patients having one of the following risk factors: obesity, dyslipidemia, and glucose intolerance and appears to be linked to insulin resistance (e.g., as in obesity or metabolic syndrome).

Incretin hormones are hormones that provide glycemic control in response to food intake. Gastric inhibitory polypeptide (“GIP”) and glucagon-like peptide-1 (“GLP-1”) are primary incretin hormones secreted from small intestinal L cells and K cells, respectively, on ingestion of glucose or nutrients to stimulate insulin secretion from pancreatic β cells. GIP and GLP-1 undergo degradation by dipeptidyl peptidase-4 (DPP-4), and rapidly lose their biological activities (see, e.g., Y. Sieno, et. al, Journal of Diabetes Investigation 2013, 4, 108-130).

The actions of GIP and GLP-1 are believed to be mediated by their specific receptors, the GIP receptor (GIPR) and the GLP-1 receptor (GLP-1R), respectively, which both belong to the G-protein coupled receptor family and are expressed in pancreatic β-cells, as well as in various tissues and organs. GLP-1 activities include, without limitation, stimulation of insulin synthesis and secretion, inhibition of glucagon secretion, and inhibition of food intake. GIP activities include, without limitation, stimulation of glucose-dependent insulin secretion, an increase in β-cell mass, stimulation of glucagon secretion, and a decrease in gastric acid secretion. See, e.g., WO 2016/131893.

GLP-1 and GLP-1 analogues, acting as agonists at the GLP-1 receptor, have been shown to be effective in glycemic control, e.g., type-2 diabetes. See, e.g., WO 2016/131893. In addition to their insulinotropic effects, GIP and GLP-1 are believed to be involved in various biological processes in different tissues and organs that express GIPR and GLP-1R, including, e.g., the pancreas, fat, bone, brain, heart, kidney, eye, nerves, and liver. By way of example, investigations using mice lacking GIPR and/or GLP-1R, as well as mice lacking DPP-4, showed involvement of GIP and GLP-1 in divergent biological activities. The results of these investigations point to involvement of GIP and GLP-1 in treating and/or preventing diabetes-related microvascular complications (e.g., retinopathy, nephropathy and neuropathy) and macrovascular complications (e.g., coronary artery disease, peripheral artery disease and cerebrovascular disease), as well as diabetes-related comorbidity (e.g., obesity, non-alcoholic fatty liver disease, bone fracture and cognitive dysfunction). See, e.g., Sieno at page 108.

### SUMMARY

This disclosure features chemical entities (e.g., a compound or a pharmaceutically acceptable salt and/or hydrate and/or prodrug of the compound) that modulate (e.g., agonize or partially agonize or antagonize) glucagon-like peptide-1 receptor (“GLP-1R”) and/or the gastric inhibitory polypeptide receptor (“GIPR”). The chemical entities are useful, e.g., for treating a subject (e.g., a human) having a disease, disorder, or condition in which modulation (e.g., agonism, partial agonism or antagonism) of GLP-1R and/or GIPR activities is beneficial for the treatment or prevention of the underlying pathology and/or symptoms and/or progression of the disease, disorder, or condition. In some embodiments, the modulation results in an enhancement of (e.g., an increase in) existing levels (e.g., normal or below normal levels) of GLP-1R and/or GIPR activity (e.g., signaling). In some embodiments, the chemical entities described herein further modulate (e.g., attenuate, uncouple) β-arrestin signaling relative to what is observed with the native ligand. This disclosure also features compositions as well as other methods of using and making the said chemical entities.

An “agonist” of GLP-1R includes compounds that, at the protein level, directly bind or modify GLP-1R such that an activity of GLP-1R is increased, e.g., by activation, stabilization, altered distribution, or otherwise relative to GLP-1R activity in absence of ligand.

Certain compounds described herein that agonize GLP-1R to a lesser extent (partial agonists) than a GLP-1R full agonist (e.g. native GLP-1) can function in assays as antagonists as well as agonists. These compounds antagonize activation of GLP-1R by a GLP-1R full agonist because they prevent the full effect of GLP-1R interaction. However, the compounds also, on their own, activate some GLP-1R activity, typically less than a corresponding amount of the GLP-1R full agonist. Such compounds are sometimes referred to herein as “partial agonists of GLP-1R”.

An “antagonist” of GLP-1R includes compounds that, at the protein level, directly bind or modify GLP-1R such that an activity of GLP-1R is decreased, e.g., by inhibition, blocking or dampening agonist-mediated responses, altered distribution, or otherwise relative to GLP-1R activity in absence of ligand.

In some embodiments, the compounds described herein are agonists (e.g. full agonists) of GLP-1R. In other embodiments, the compounds described herein are partial agonists or antagonists of GLP-1R.

An “agonist” of GIPR includes compounds that, at the protein level, directly bind or modify GIPR such that an activity of GIPR is increased, e.g., by activation, stabilization, altered distribution, or otherwise relative to GIPR activity in absence of ligand.

Certain compounds described herein that agonize GIPR to a lesser extent than native GIP (s full agonist) can function as antagonists as well as agonists. These compounds are partial antagonists as they reduce activation of GIPR by native GIP because they reduce the full effect of native GIP. However, the compounds also, on their own, activate some GIPR activity, typically less than a corresponding amount of native GIP. Such compounds are sometimes referred to herein as “partial agonists of GIPR”.

An “antagonist” of GIPR includes compounds that, at the protein level, directly bind or modify GIPR such that an activity of GIPR is decreased, e.g., by inhibition, blocking or dampening agonist-mediated responses, altered distribution, or otherwise relative to GIPR activity in absence of ligand.

In some embodiments, the compounds described herein are agonists (e.g. full agonists) of GIPR. In other embodiments, the compounds described herein are partial agonists of GIPR. In still other embodiments, the compounds described herein are or antagonists of GIPR.

In some embodiments, the compounds described herein are full agonists of both GLP-1R and GIPR. In some embodiments, the compounds described herein are partial agonists of both GLP-1R and GIPR. This disclosure also contemplates chemical entities that (i) agonize one of GLP-1R and GIPR (e.g., GLP-1R); and antagonize the other of GLP-1R and GIPR (e.g., GIPR). For example, this disclosure contemplates chemical entities that (i) fully agonize one of GLP-1R and GIPR (e.g., GLP-1R); and partially aginize or antagonize the other of GLP-1R and GIPR (e.g., GIPR). In certain embodiments, the chemical entities that are full agonists of GLP-1R and partial agonists or antagonists of GIPR; or that are partial agonists or antagonists of GLP-1R and full agonists of GIPR.

Generally, a receptor exists in an active (Ra) and an inactive (Ri) conformation. Certain compounds that affect the receptor can alter the ratio of Ra to Ri (Ra/Ri). For example, a full agonist increases the ratio of Ra/Ri and can cause a “maximal”, saturating effect. A partial agonist, when bound to the receptor, gives a response that is lower than that elicited by a full agonist (e.g., an endogenous agonist). Thus, the Ra/Ri for a partial agonist is less than for a full agonist. However, the potency of a partial agonist may be greater or less than that of the full agonist.

In some embodiments, the chemical entities described herein further modulate (e.g, reduce, e.g., attenuate, disrupt, inhibit) β-arrestin coupling and/or b-arrestin signaling and GLP-1R and/or GIPR internalization. In certain embodiments, the chemical entities described herein modulate (e.g., reduce, e.g., attenuate, disrupt, inhibit) recruitment of β-arrestin to a G-protein coupled receptor (e.g., GLP-1R). In certain embodiments, the chemical entities described herein modulate (e.g., reduce, e.g., attenuate, disrupt, inhibit) formation of a complex (“coupling”) (e.g., a signaling complex) between β-arrestin and a G-protein coupled receptor (e.g., GLP-1R or GIPR). The effects of the chemical entities described herein on β-arrestin signaling and associated downstream processes (e.g., those delineated above) can be assessed using conventional methods, e.g., PathHunter β-arrestin Assay for determining β-arresting coupling (see Examples section). By way of example, a value of β-arrestin (GLP-1R) EC50<1 μM in the aforemented assay indicates a compound that induces β-arrestin recruitment to GLP-1R. GLP-1, the native ligand for GLP-1R, and therapeutic analogs thereof, such a liraglutide, are potent recruiters of β-arrestin. As another example, a value of β-arrestin (GLP1R) EC₅₀>1 μM (e.g., >10 μM) indicates a compound that does not substantially induce β-arrestin recruitment to GLP-1R Therapeutic agents that modulate G-protein coupled receptors (e.g., GLP-1R and/or GIPR) can produce a variety of effects depending on the degree of cAMP activation versus β-arrestin-based signaling. It has been shown that b-arrestin coupling is a key strep in receptor internalization and subsequent de-sensitization and attenuation of signaling. Both GLP-1 (and the liraglutide analog) and GIP have been shown to produce rapid receptor internalization. Thus, compounds that activate GLP-1R and/or GIPR CAMP signaling but do not substantially couple to β-arrestin have the potential to prolong receptor signaling and extend pharmacological benefits. In some embodiments, the chemical entities described herein exhibit relatively strong GLP-1R and/or GIPR mediated CAMP activation with minimal or no detectable β-arrestin coupling. In some embodiments, the chemical entities described herein exhibit relatively strong GLP-1R mediated CAMP activation and no or little GIPR mediated cAMP activation with minimal or no detectable β-arrestin coupling.

In some embodiments, the compounds described herein show reduced activity in a rodent aversion model, while maintaining a relatively high potency in glucose clearance assay. Aversion models, such as conditioned taste aversion, are commonly used to identify compounds with adverse effects such as nausea. Nausea causing agents, such a Exendin-4 and liraglutide, are know to have a strong signal in conditioned taste aversion models. Advantageously, some of the chemical entities described herein are therefore expected to have a reduced likelihood of producing unwanted side effects, such as nausea, when administered to a patient, while maintaining full pharmacological benefit.

Accordingly, in one aspect, this disclosure features peptide-based chemical entities (e.g., N-protected peptide-based chemical entities; e.g., peptide-based chemical entities having from 30-50 amino acids, 30-45 amino acids, 30-40 aminoacids, 35-40 amino acids; e.g., an N-protected peptide-based chemical entities having from 30-50 amino acids, 30-45 amino acids, 30-40 amino acids, 35-40 amino acids). The peptide-based chemical entities modulate (e.g., agonize or partially agonize or antagonize) glucagon-like peptide-1 receptor (“GLP-1R”) and/or the gastric inhibitory polypeptide receptor (“GIPR”) and optionally further modulate (e.g., uncouple, attenuate,) β-arrestin signaling and/or aversion as described herein.

In some embodiments, the peptide-based chemical entities agonize or partially agonize GLP-1R.

In some embodiments, the peptide-based chemical entities agonize or partially agonize or antagonize GIPR.

In some embodiments, said peptide-based chemical entities reduce (e.g., uncouple, attenuate, inhibit) β-arrestin signaling; e.g., reduce (e.g., uncouple, attenuate, inhibit) the recruitment of β-arrestin to a G-protein coupled receptor (e.g., GLP-1R); e.g., reduce (e.g, attenuate, disrupt, inhibit) the formation of a complex (e.g., a signaling complex) between β-arrestin and a G-protein coupled receptor (e.g., GLP-1R).

In certain embodiments, the peptide-based chemical entities:
agonize or partially agonize GLP-1R;
agonize or partially agonize or antagonize GIPR;
reduce (e.g., uncouple, attenuate, inhibit) β-arrestin signaling; e.g., reduce (e.g., attenuate, disrupt, inhibit) the recruitment of β-arrestin to a G-protein coupled receptor (e.g., GLP-1R and/or GIPR); e.g., reduce (e.g., attenuate, disrupt, inhibit) the formation of a complex (e.g., a signaling complex) between β-arrestin and a G-protein coupled receptor (e.g., GLP-1R and/or GIPR); and
stimulate glucose clearance in vivo (GTT test) without causing aversion.

In some embodiments, the peptide-based chemical entities exhibit a value of CAMP (GLP1R) EC50 of less than about 10 nM, 5 nM, or 1 nM (e.g., less than about 1 nM).

In some embodiments, the peptide-based chemical entities exhibit a value of cAMP (GIPR) EC50 of less than about 100 nm, 50 nM, or 10 nM (e.g., less than about 10 nM).

In some embodiments, the peptide-based chemical entities inhibit GIP induced CAMP production (GIPR antagonism).

In some embodiments, the compounds described herein exhibit a value of β-arrestin (GLP1R) EC50>1 μM.

In some embodiments, the peptide-based chemical entities:
exhibit a value of cAMP (GLP1R) EC50 of less than about 10 nM, 5 nM, or 1 nM (e.g., less than about 1 nM);
exhibit a value of cAMP (GIPR) EC50 of less than about 100 nm, 50 nM, or 10 nM (e.g., less than about 10 nM) or are GIPR antagonists;
exhibit a value of β-arrestin (GLPIR) EC50>1 μM; and
stimulate glucose clearance in vivo (GTT test) without causing aversion (nausea)

In one aspect, the featured peptide-based chemical entities include compounds of Formula (IAA), or a pharmaceutically acceptable salt thereof:

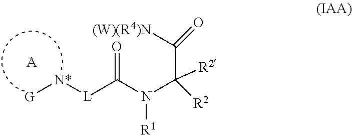

in which ring A, G, L, W, R¹, R², R^{2′}, and R⁴ can be as defined anywhere herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing, partially agonizing, or antagonizing) GLP-1R and/or GIPR activities are featured that include contacting GLP-1R and/or GIPR with a chemical entity described herein (e.g., a compound described generically or specifically herein or a pharmaceutically acceptable salt thereof or compositions containing the same). Methods include in vitro methods, e.g., contacting a sample that includes one or more cells, each independently comprising one or more of GLP-1R and/or GIPR with the chemical entity. Methods can also include in vivo methods; e.g., administering the chemical entity to a subject (e.g., a human) having a disease, disorder, or condition in which modulation of GLP-1R and/or GIPR signaling is beneficial for the treatment of the underlying pathology and/or symptoms and/or progression of the disease, disorder, or condition (e.g., diabetes; e.g., NASH; e.g., obesity). In vivo methods include, but are not limited to modulating (e.g., increasing) insulin levels and modulating (e.g., decreasing) glucose levels in a subject (e.g., a human). In some of the foregoing embodiments, said methods of modulating are achieved without substantially stimulating β-arrestin signaling; e.g., without stimulating the recruitment of β-arrestin to a G-protein coupled receptor (e.g., GLP-1R); e.g., without stimulating the formation of a complex (e.g, a signaling complex) between β-arrestin and a G-protein coupled receptor (e.g., GLP-1R). In some of the foregoing embodiments, said methods of modulating are achieved without causing aversion or nausea.

In a further aspect, methods of treatment of a disease, disorder, or condition are featured, in which modulation of GLP-1R and/or GIPR signaling is beneficial for the treatment of the underlying pathology and/or symptoms and/or progression of the disease, disorder, or condition. The methods include administering to a subject in need of such treatment an effective amount of a chemical entity described herein (e.g., a compound described generically or specifically herein, a pharmaceutically acceptable salt thereof, or compositions containing the same).

In another aspect, this disclosure features methods of treating a subject having a disease, disorder, or condition in which modulation of GLP-1R and/or GIPR signaling is beneficial for the treatment of the underlying pathology and/or symptoms and/or progression of the disease, disorder, or condition. The methods include administering a chemical entity described herein (e.g., a compound described generically or specifically herein, a pharmaceutically acceptable salt thereof or compositions containing the same) in an amount effective to treat the disease, disorder, or condition.

In a further aspect, methods of treatment are featured that include administering to a subject chemical entity described herein (e.g., a compound described generically or specifically herein, a pharmaceutically acceptable salt thereof, or compositions containing the same). The methods include administering the chemical entity in an amount effective to treat a disease, disorder, or condition, wherein modulation of GLP-1R and/or GIPR is beneficial for the treatment of the underlying pathology and/or symptoms and/or progression of the disease, disorder, or condition, thereby treating the disease, disorder, or condition.

In another aspect, methods of treatment can further include:
(i) administering a first therapeutic agent that modulates (e.g., agonizes, partially agonizes, or antagonizes) GLP-1R and/or GIPR to a subject as defined anywhere herein;
(ii) determining that the subject is suffering from one or more side effects (e.g., aversion, nausea or vomiting); and
(iii) ceasing administration of the first therapeutic agent and administering a chemical entity as described herein (e.g., a compound having formula (IAA), (IA), or (I)).

Non-limiting examples of such diseases, disorders, and conditions include metabolic syndrome; diabetes (e.g., type 2 diabetes); obesity; obesity-related disorders; impaired glucose tolerance; insulin resistance; non-alcoholic steatohepatitis (NASH); fatty liver disease; steatohepatitis; and other forms of inflammation in metabolically important tissues including, liver, fat, pancreas, kidney, and gut.

Other non-limiting examples of such diseases, disorders, and conditions include neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome).

Still other non-limiting examples of such diseases, disorders, and conditions include bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging pregnancy, protection against bone fractures, and malnutrition.

In certain embodiments, the disease, disorder, or condition is diabetes.

In other embodiments, the disease, disorder, or condition is NASH.

In still other embodiments, the disease, disorder, or condition is obesity.

In other embodiments, the disease, disorder, or condition is Alzheimer's disease (AD) or Parkinson's disease (PD).

In still other embodiments, the disease, disorder, or condition is a bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging or pregnancy.

In certain embodiments, the chemical entities described herein are useful for protection against bone fractures.

The methods described herein can further include treating one or more conditions associated, co-morbid or sequela with any one or more of the conditions described herein. By way of non-limiting example, the methods can further include treating one or more conditions that are co-morbid or sequela with diabetes (e.g., type 2 diabetes), such as obesity, obesity-related disorders, metabolic syndrome, impaired glucose tolerance; insulin resistance; cardiovascular risk factors. (e.g., coronary artery disease, peripheral artery disease, cerebrovascular disease, hypertension, and risk factors related to unmanaged cholesterol and/or lipid levels, and/or inflammation), retinopathy, nephropathy, neuropathy, NASH, bone fracture and cognitive dysfunction.

In another aspect, this disclosure features methods for screening a candidate compound for treatment of a disease, disorder, or condition, in which modulation of GLP-1R and/or GIPR is beneficial for the treatment of the underlying pathology and/or symptoms and/or progression of the disease, disorder, or condition; the method comprising:
(a) contacting the candidate compound with (i) a β-Arrestin-coupled GPCR receptor signaling complex or (ii) one or more GPCR receptor signaling complexes that are not complexed with β-arrestin (e.g., uncoupled GLP-1R and/or uncoupled GIPR); and
(b) detecting (i) the disruption of the b-arrestin coupled complex; or (ii) detecting the formation of the signaling complex in the absence of b-arrestin coupling; wherein the candidate compound modulates (e.g., agonizes, partially agonizes,) GLP-1R and/or GIPR.

In certain embodiments, the methods further include selecting a candidate compound that exhibits a value of β-arrestin (GLP-1R) EC50>1 μM.

The methods can further include identifying the subject.

The methods can further include administering one or more other therapeutic agents (e.g., in combination with a chemical entity described herein).

Embodiments can include one of more of the following advantageous properties.

In some embodiments, the compounds described herein exhibit a value of AUC Score of 0-25% of vehicle, which corresponds to a finding of maximal decrease in glucose excursion compared to control.

In some embodiments, the compounds described herein exhibit a value of cAMP (GLP-1R) EC50 of less than about 1 nM, which is indicative that the compound is a relatively potent GLP-1R agonist.

In some embodiments, the compounds described herein exhibit a value of cAMP (GLP-1R) Emax of greater than about 80%, which is indicative that the compound is an agonist that can fully activate GLP-1R.

In some embodiments, the compounds described herein exhibit a value of cAMP (GIPR) EC50 of less than about 10 nM, which is indicative that the compoundis a relatively potent GIPR agonist.

In some embodiments, the compounds described herein exhibit a value of cAMP (GIPR) EC50 of less than about 10 nM, which is indicative that the compoundis a relatively potent GIPR antagonist.

GLP-1 and GIP are susceptible to rapid degradation by dipeptidyl peptidease-IV (DPP-IV) (see, e.g., Deacon, et al. Journal of Clinical Endocrinology & Metabolism, 1995, 80, 952-957). As such, GLP-1 and GIP have been shown to exhibit relatively short half-life times in human due to DPP-IV degradation. Advantageously, the compounds described herein exhibit relatively long half-life times in the presence of DPP-IV when compared to those of GLP-1 and GIP.

In some embodiments, the compounds described herein exhibit a value of β-arrestin coupling (GLP-1R) EC50>1 μM in a β-arrestin (GLPIR) assay that measures the formation of a complex between GLP-1R and β-arrestin in cells. A value of β-arrestin (GLP1R) EC50>1 μM indicates a compound that does not substantially induce β-arrestin recruitment to GLP-1R.

In some embodiments, the compounds described herein exhibit a value of conditioned taste aversion (“CTA”)=0.6-1.0, which indicates no measurable aversion to a compound, which is desired. A value of CTA=0.0-0.6 indicates significant aversion to a compound. The conditioned taste aversion assay measures the preference for a dilute saccharin solution that is associated with compound administration.

In some embodiments, the compounds described herein have a reduced likelihood of producing unwanted side effects when administered to a patient. A non-limiting example of such a side effect is nausea.

Other embodiments include those described in the Detailed Description, drawings, and/or in the claims.

### DESCRIPTION OF DRAWINGS

FIG. 1 depicts representative compounds of Formula (IAA).

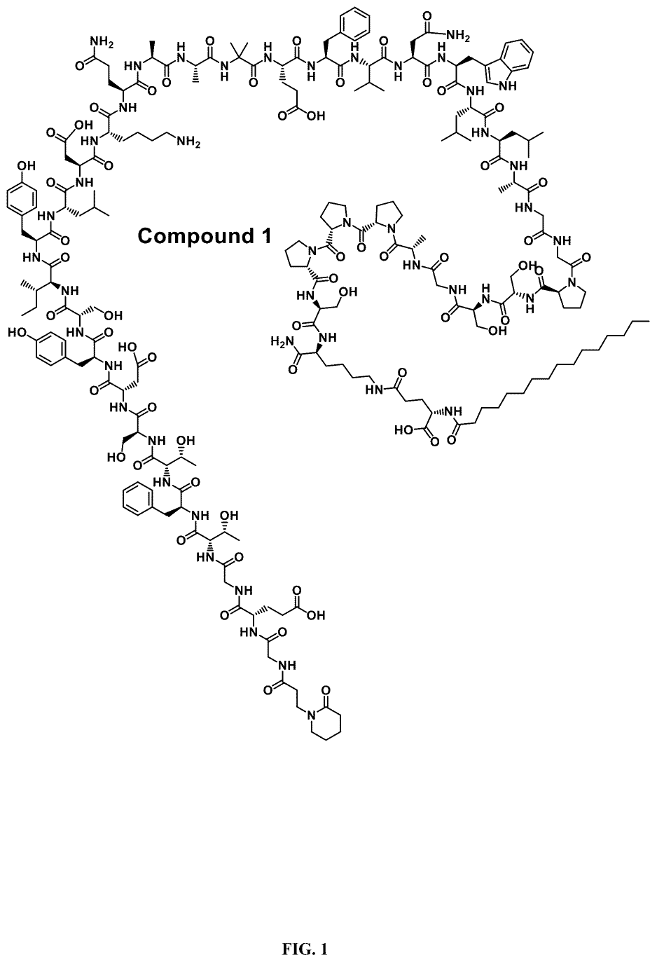

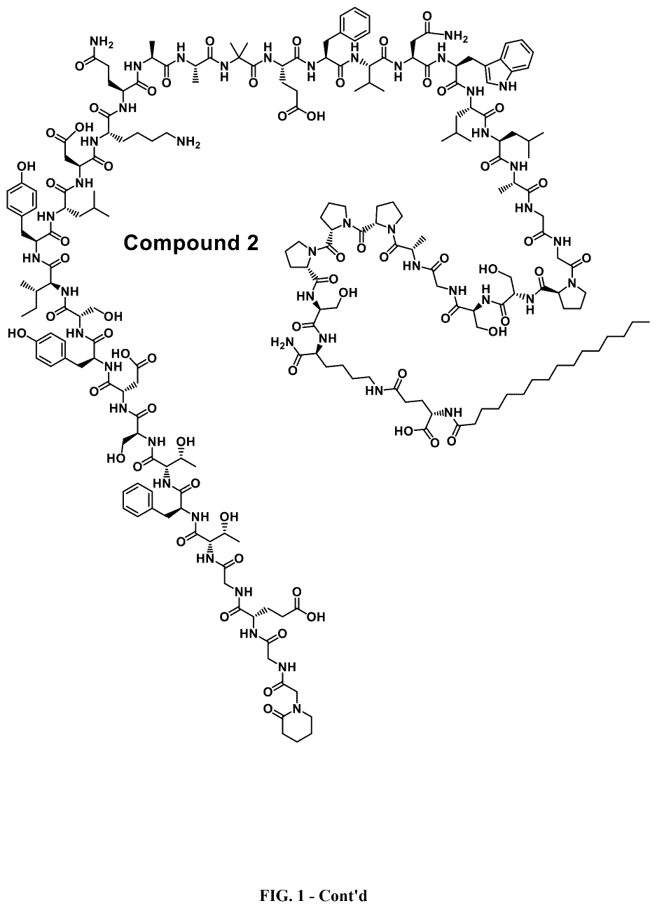

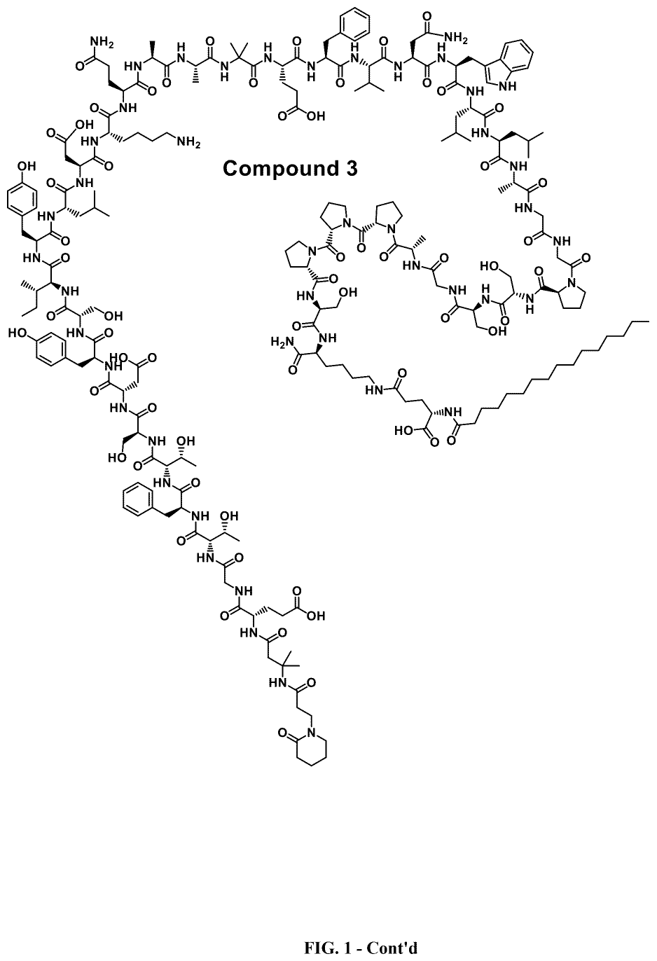

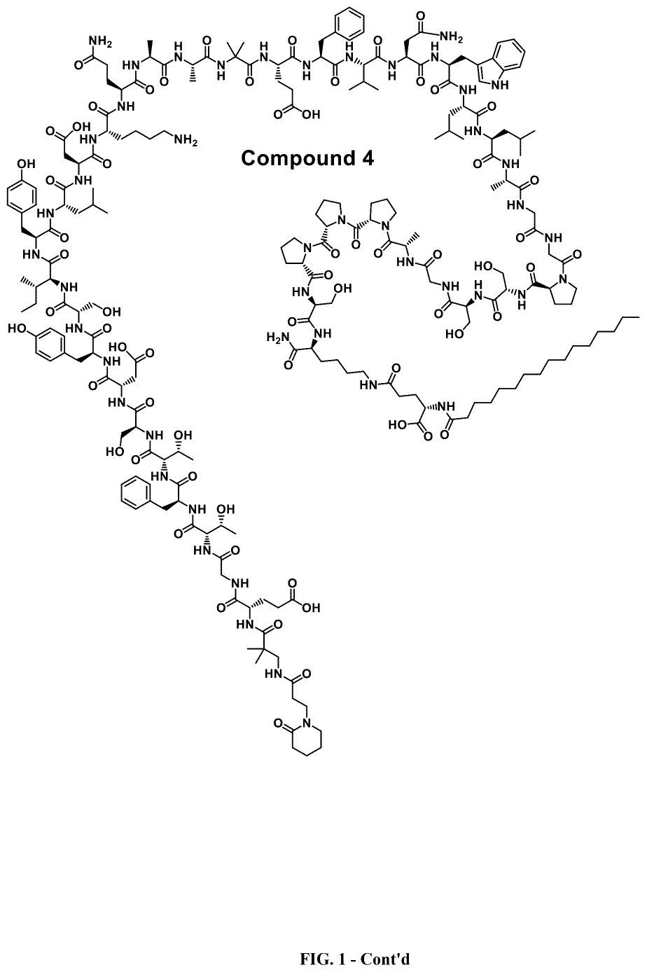

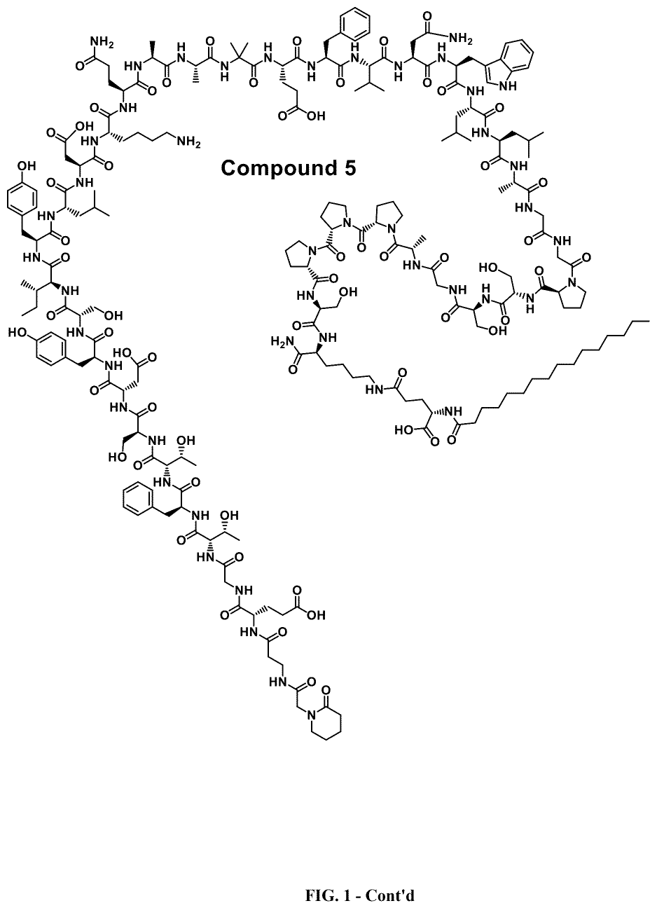

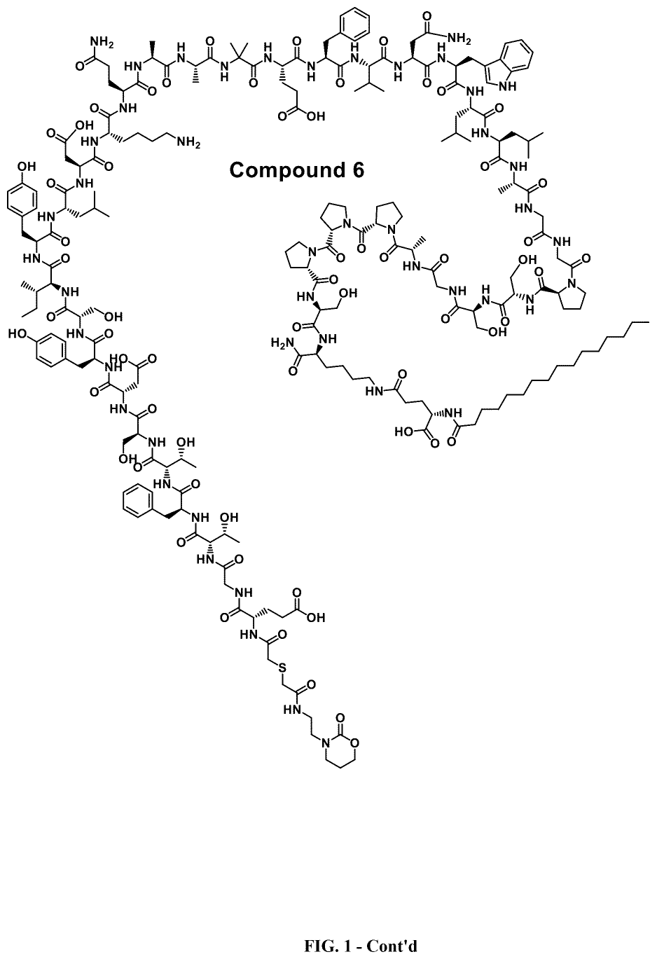

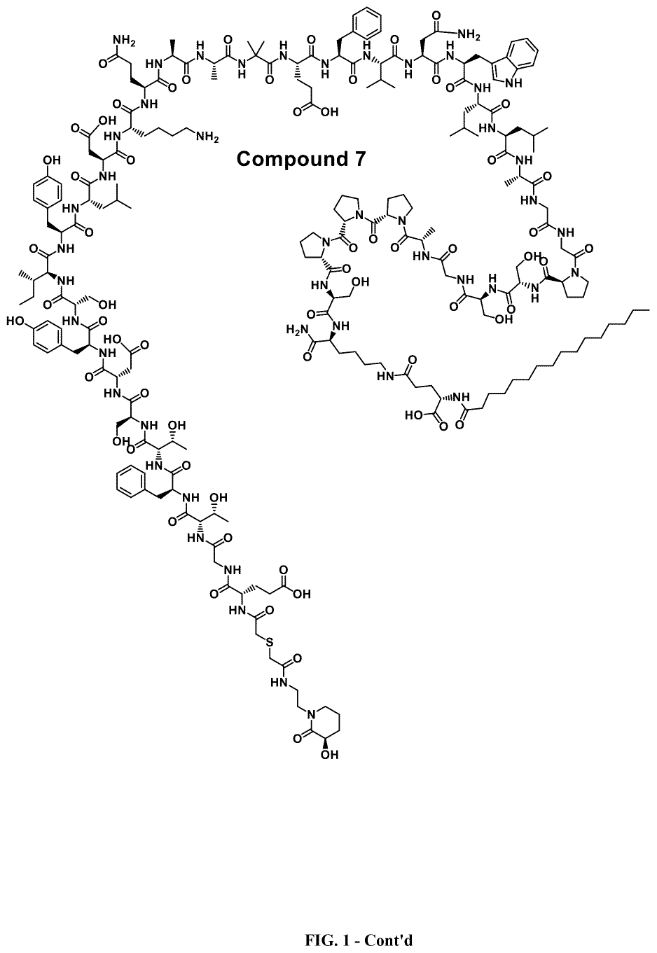

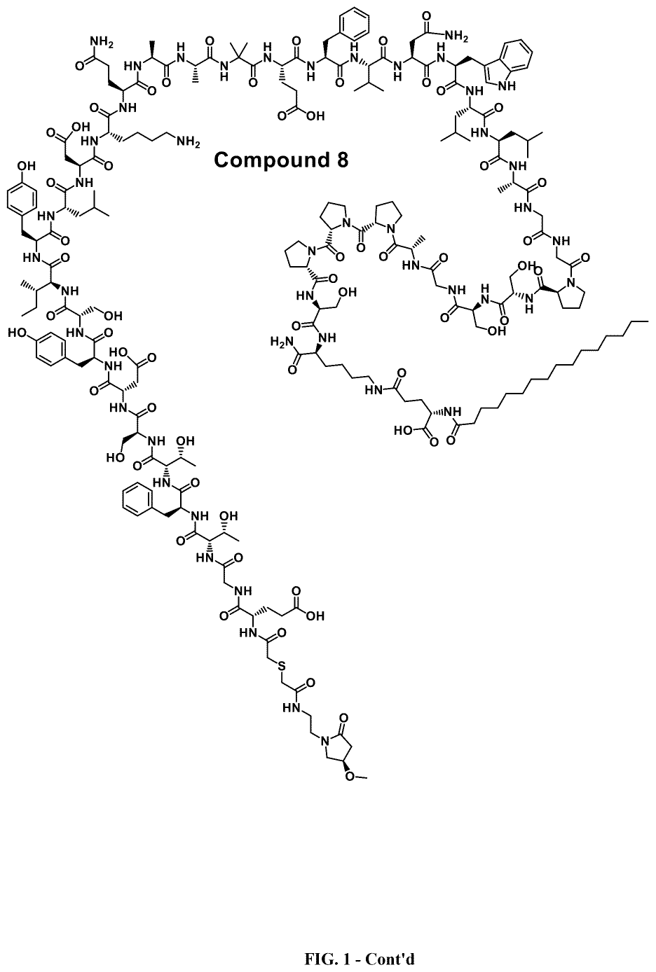

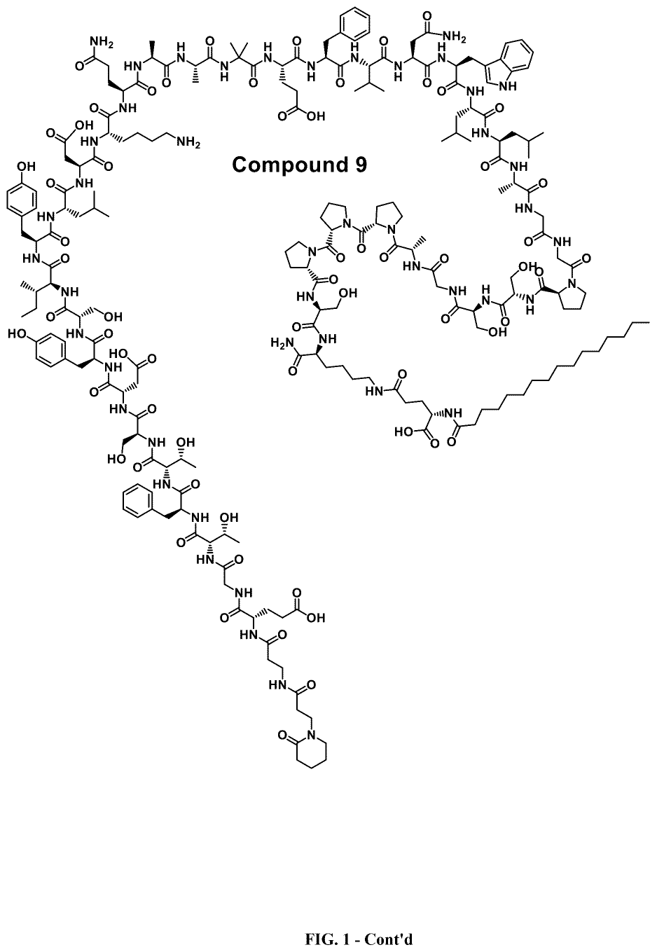

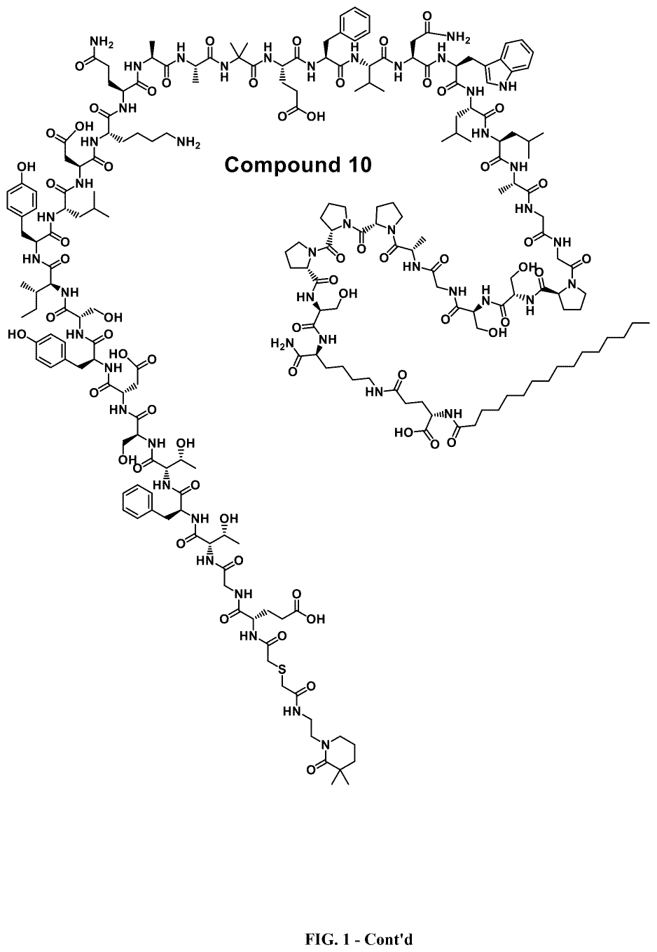

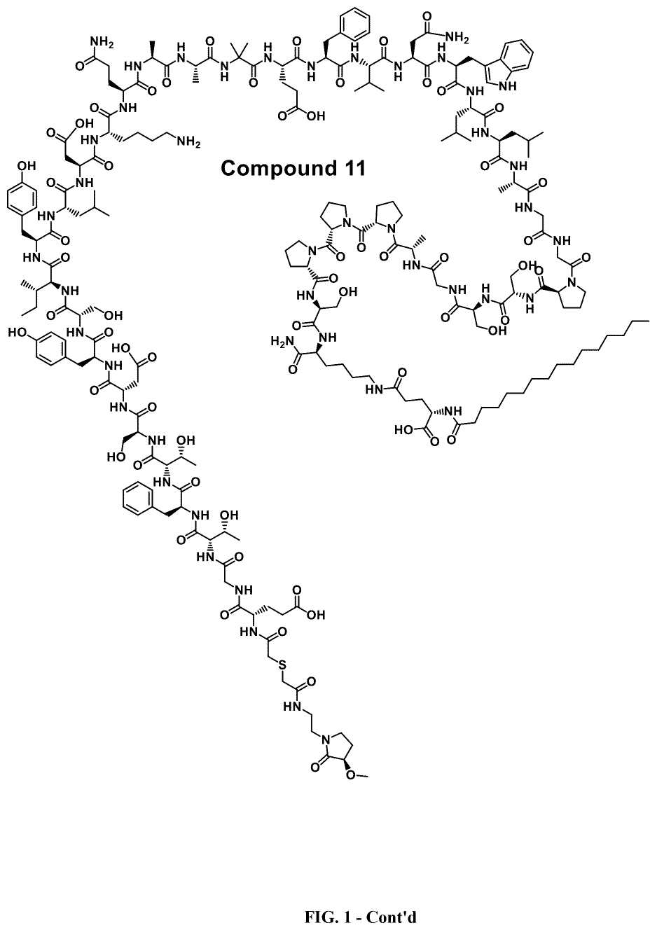

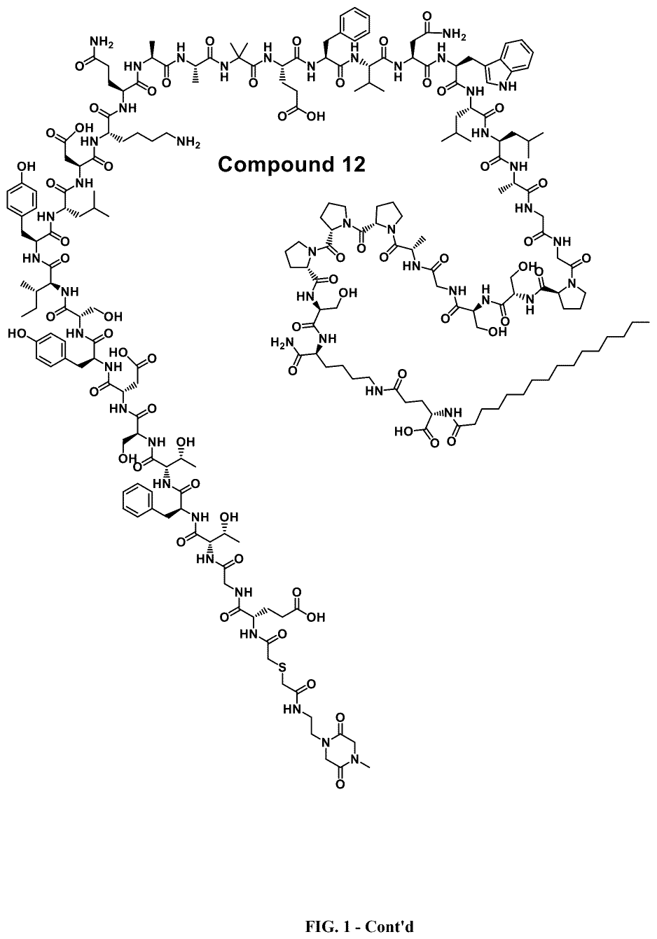

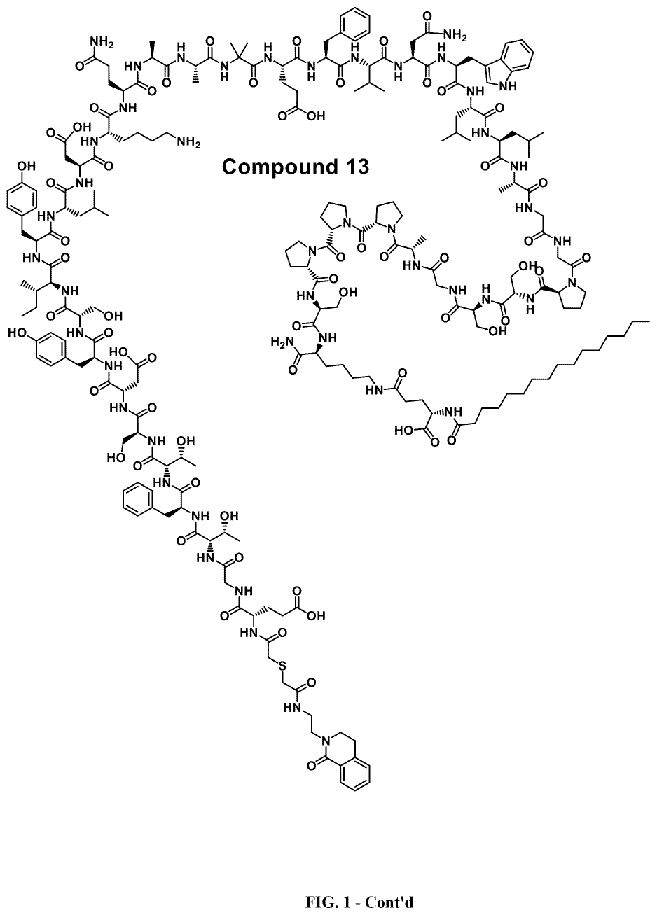

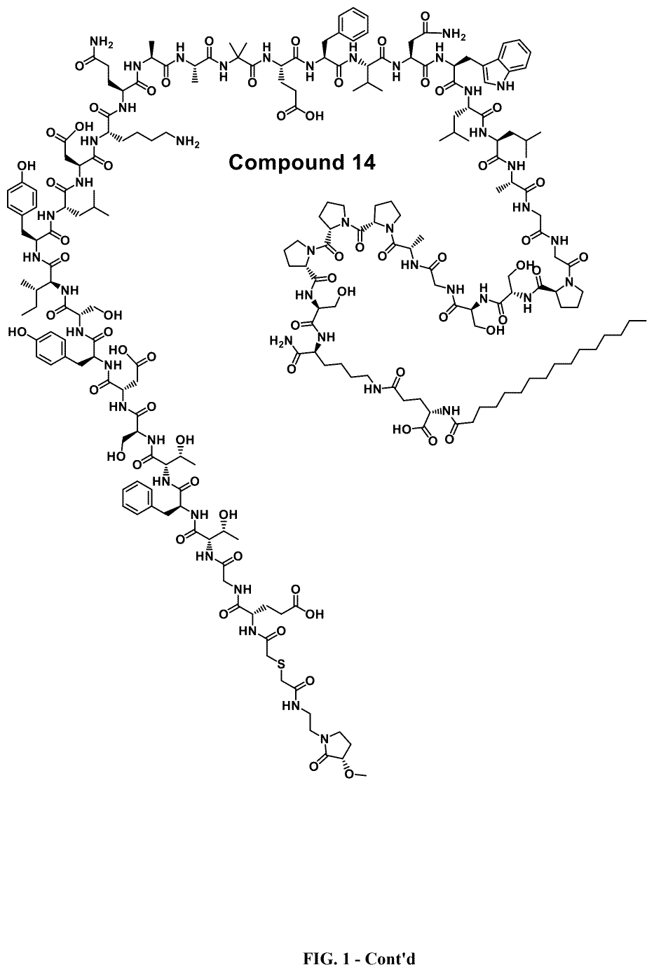

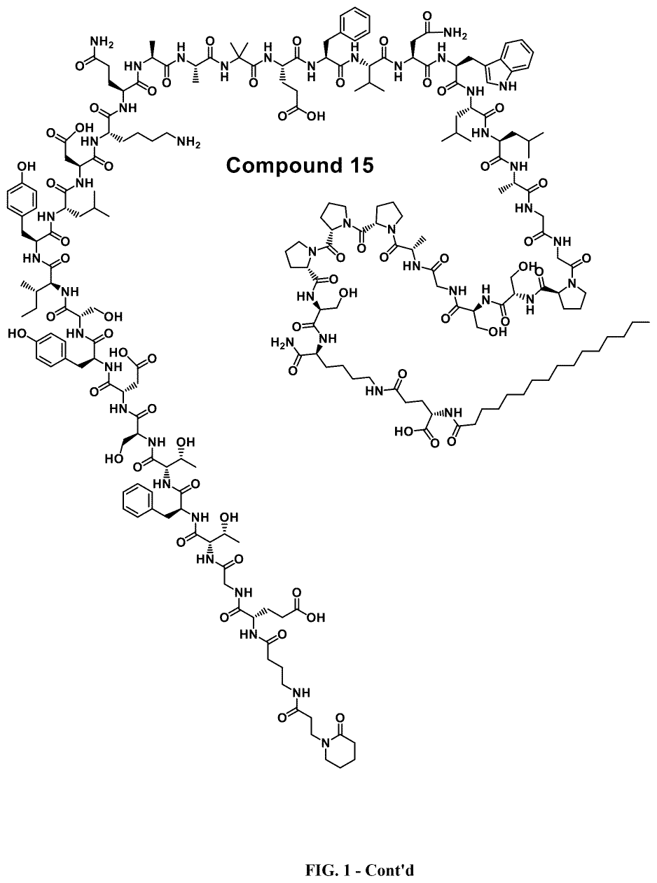

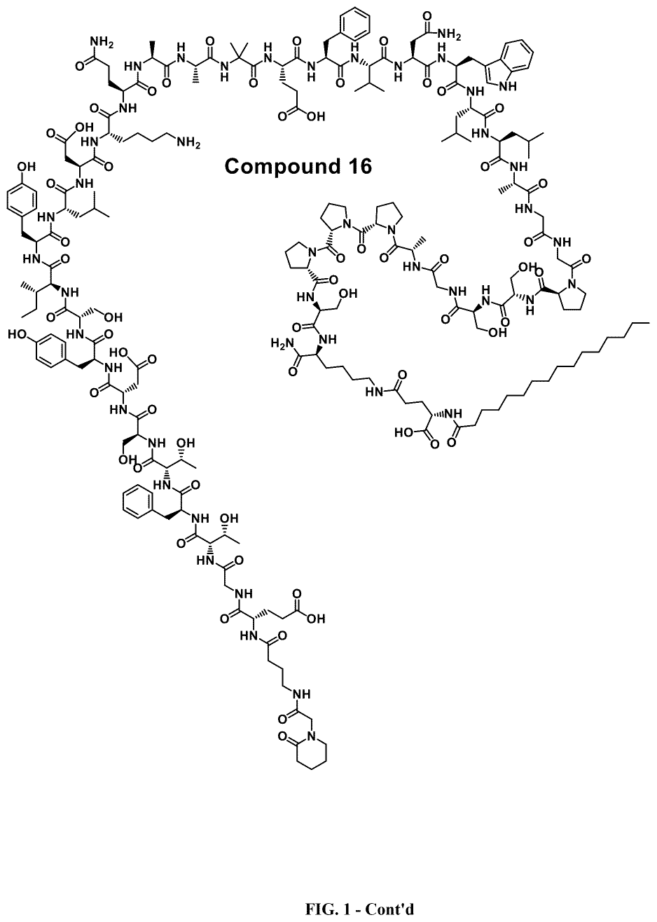

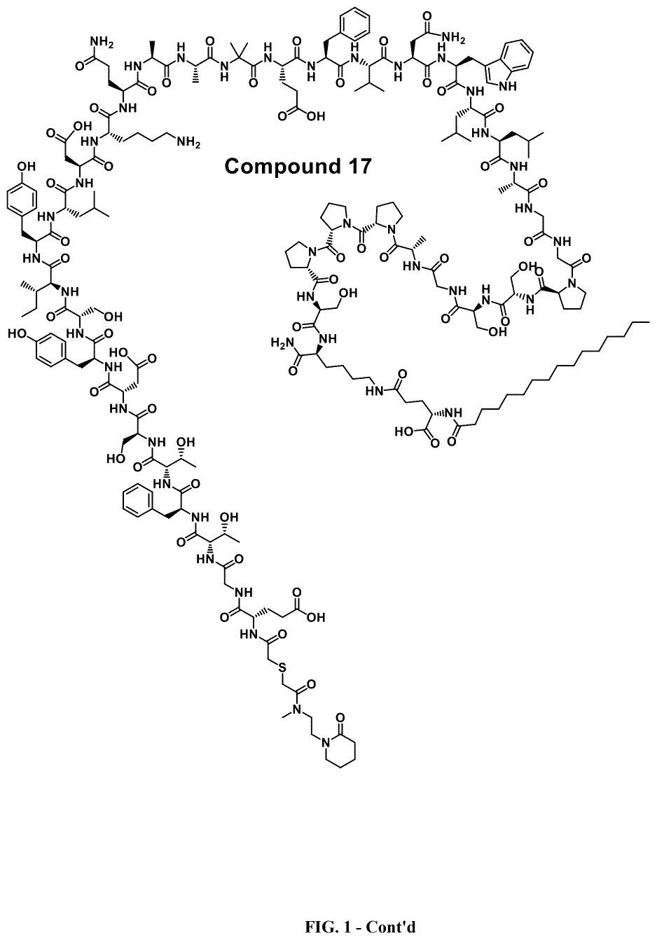

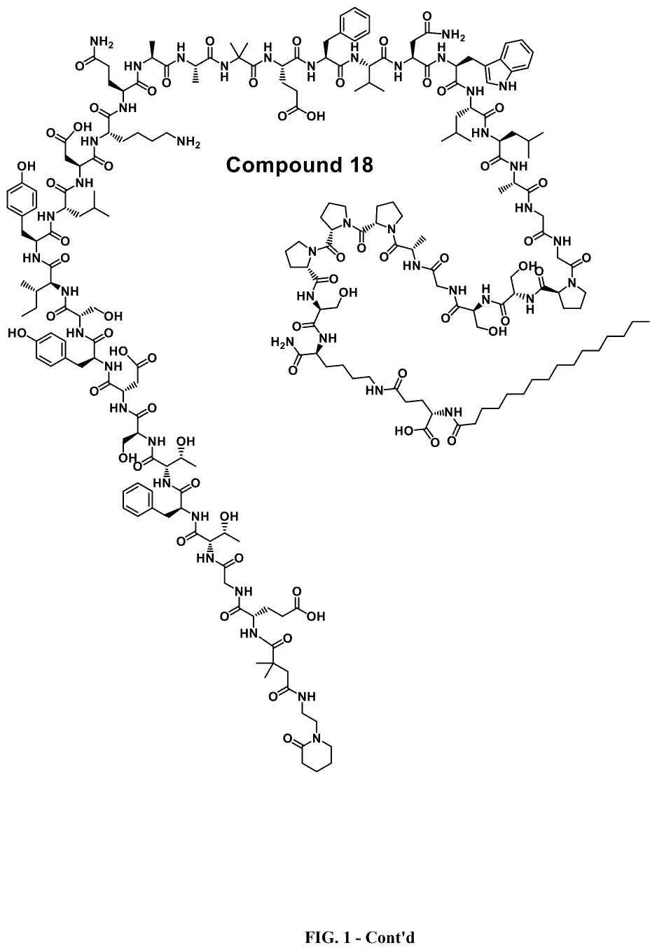

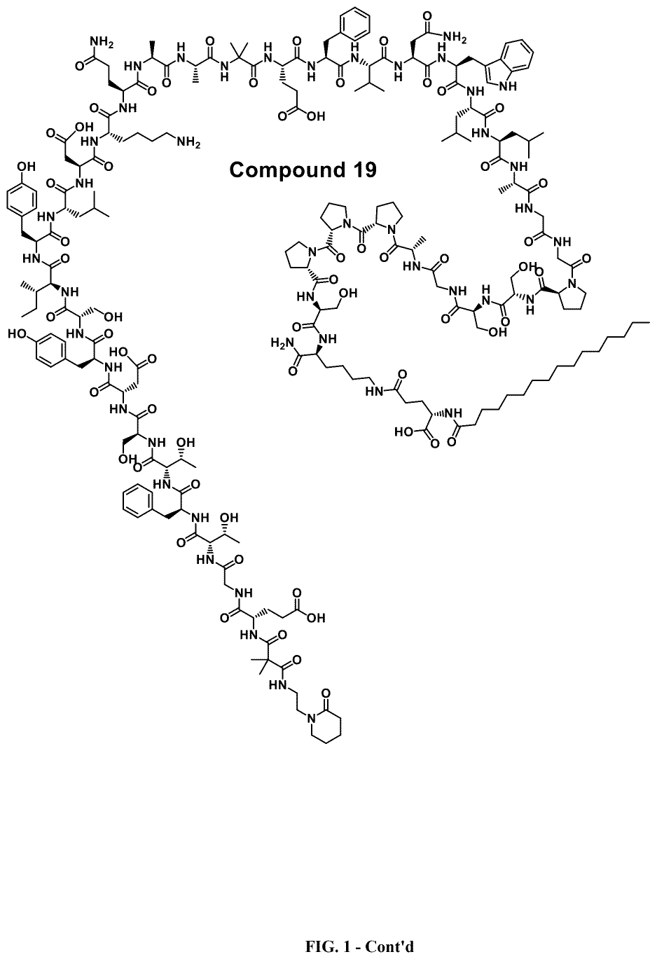

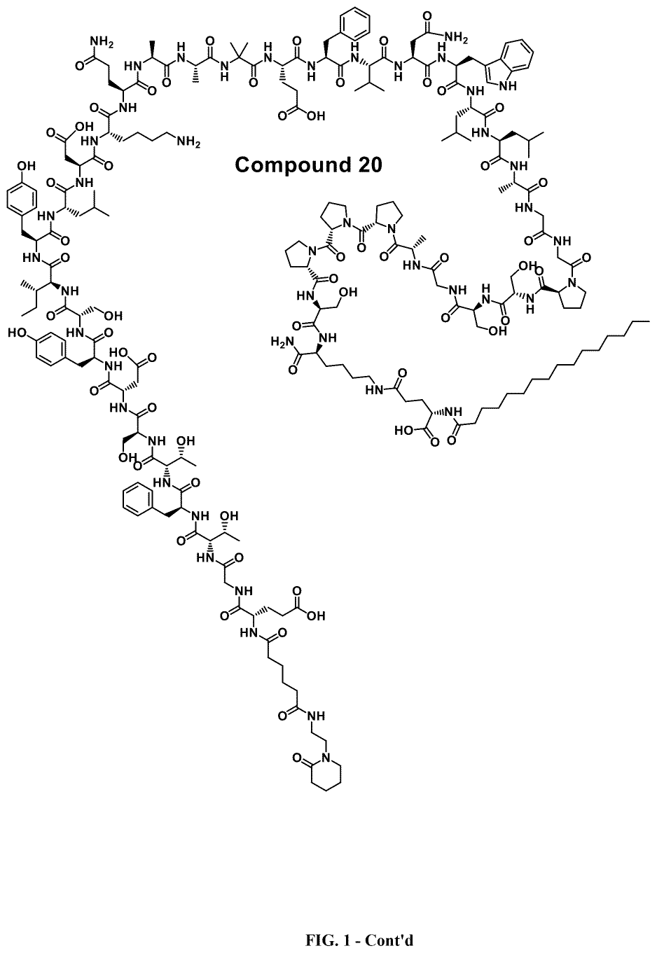

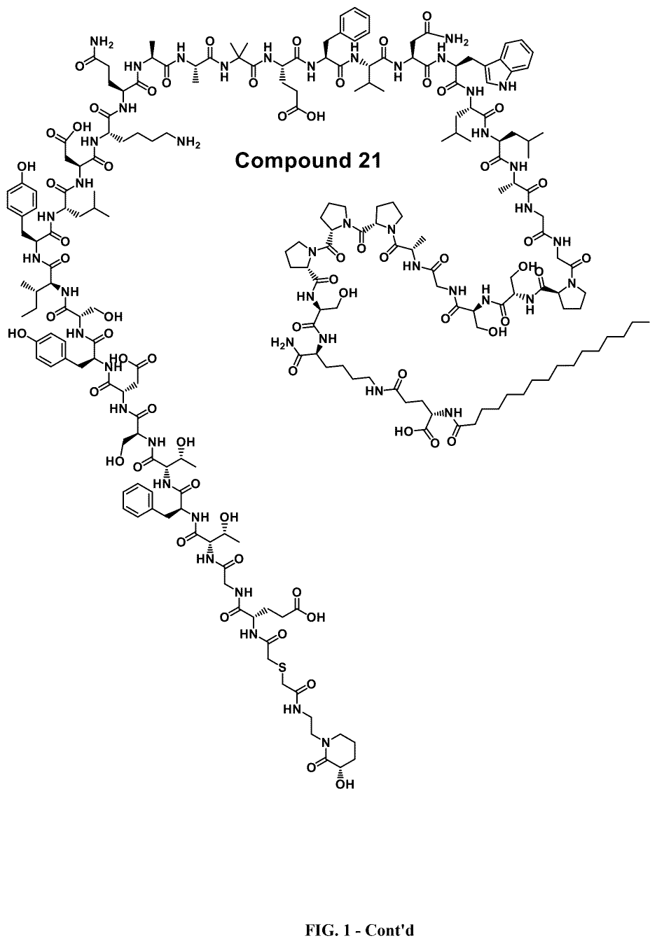

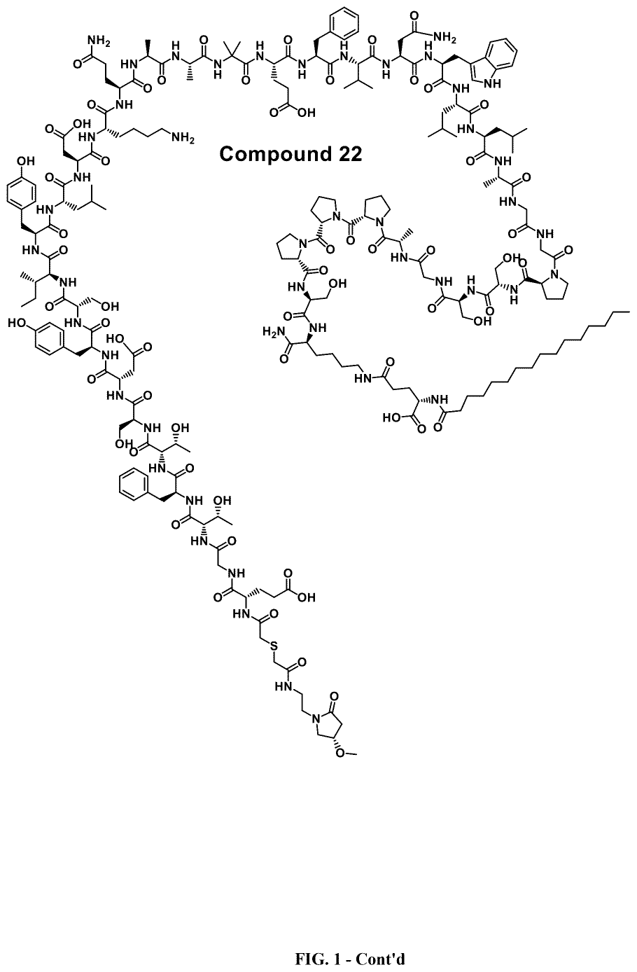

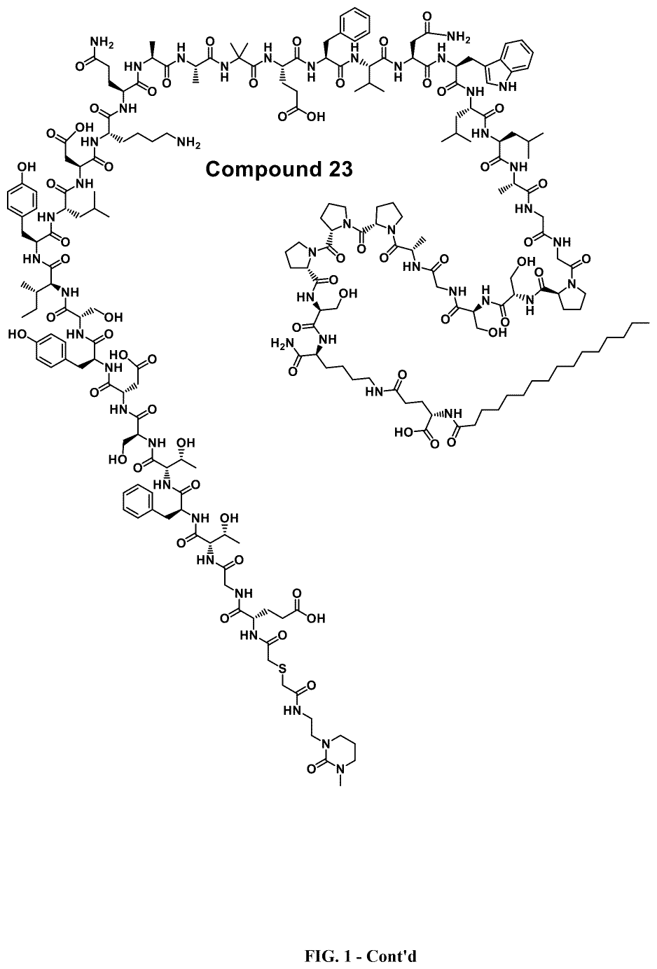

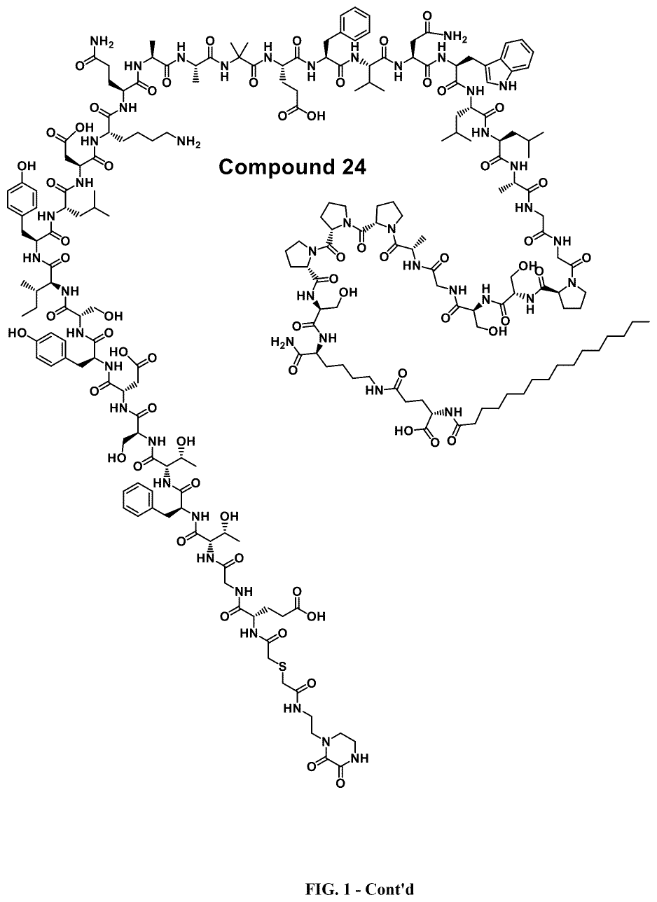

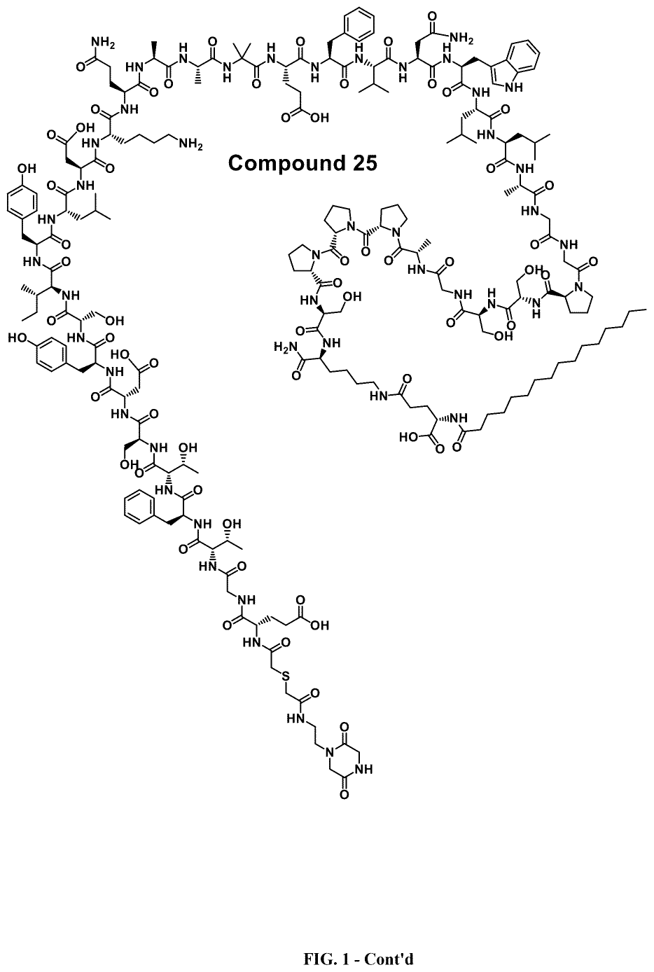

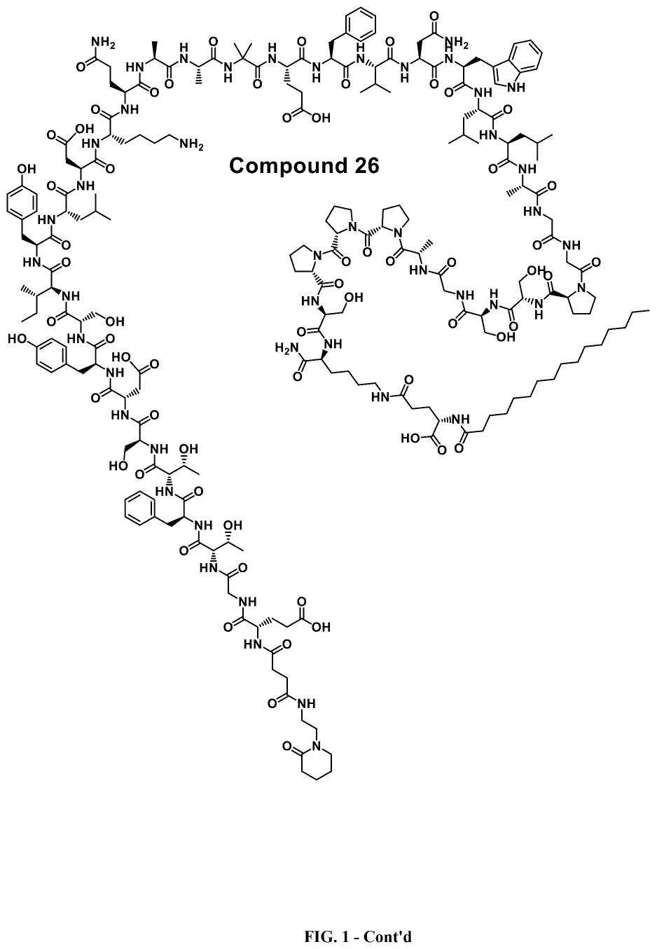

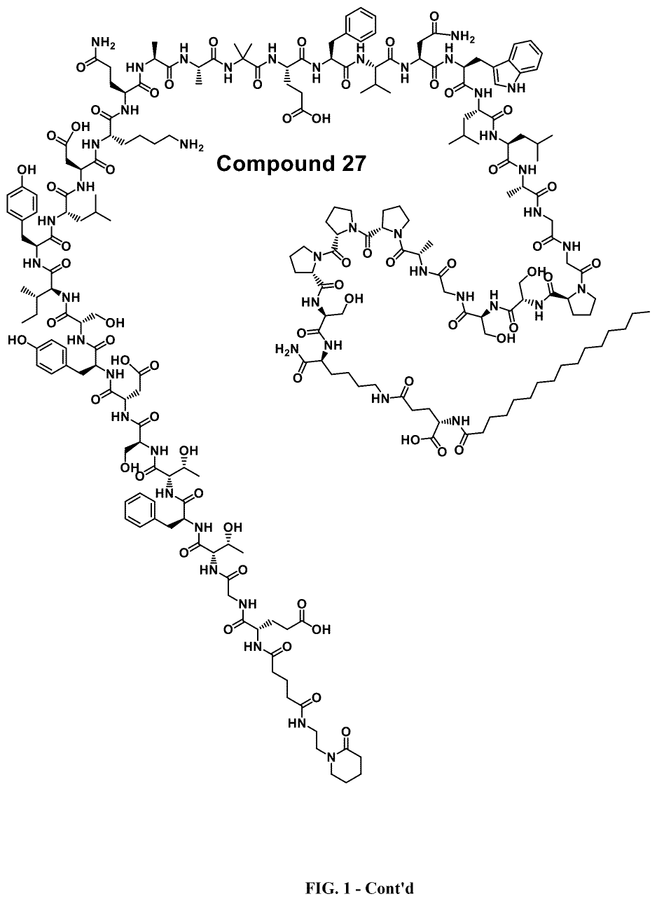

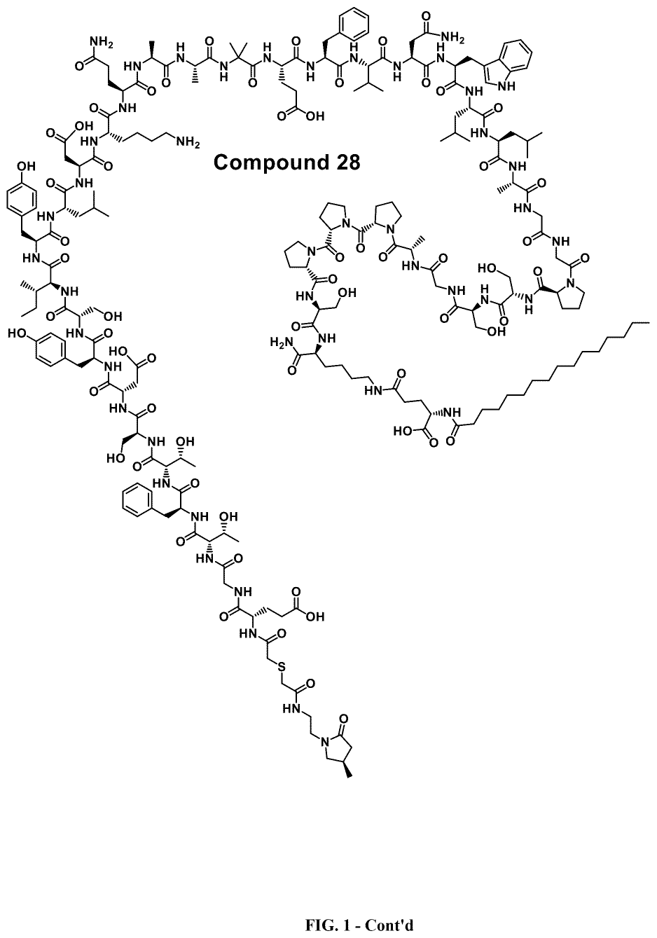

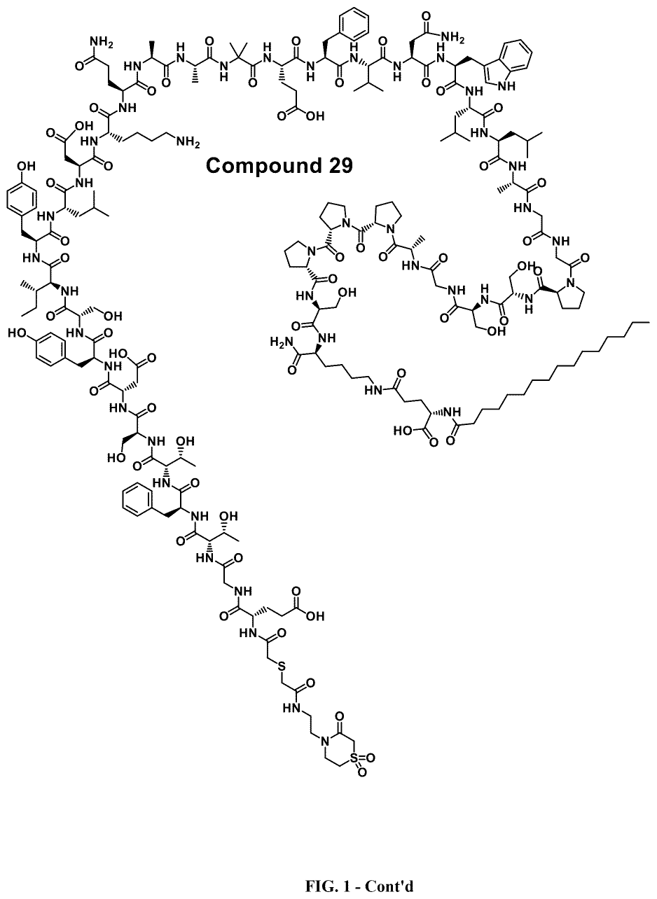

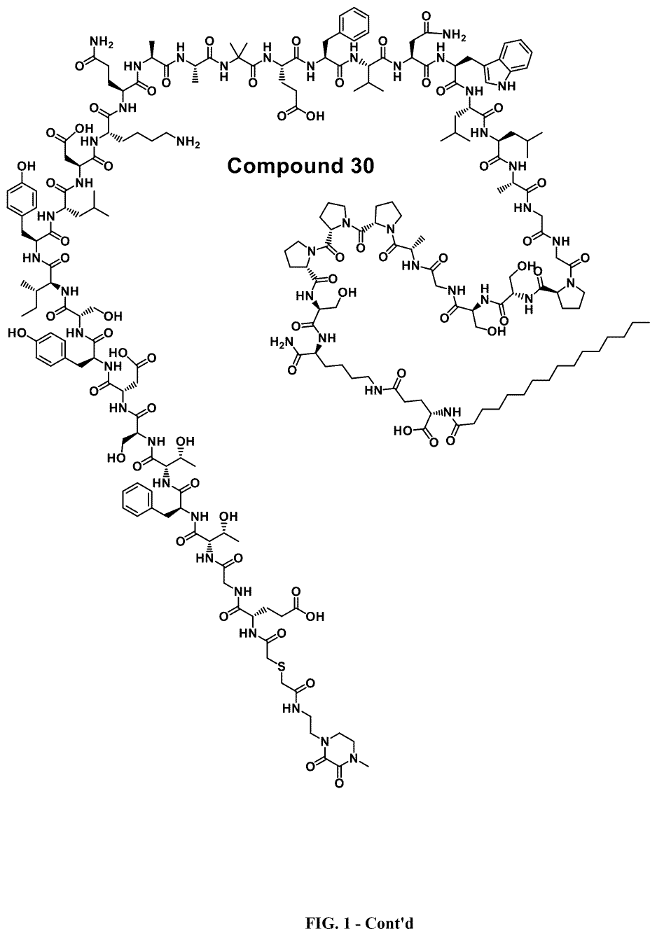

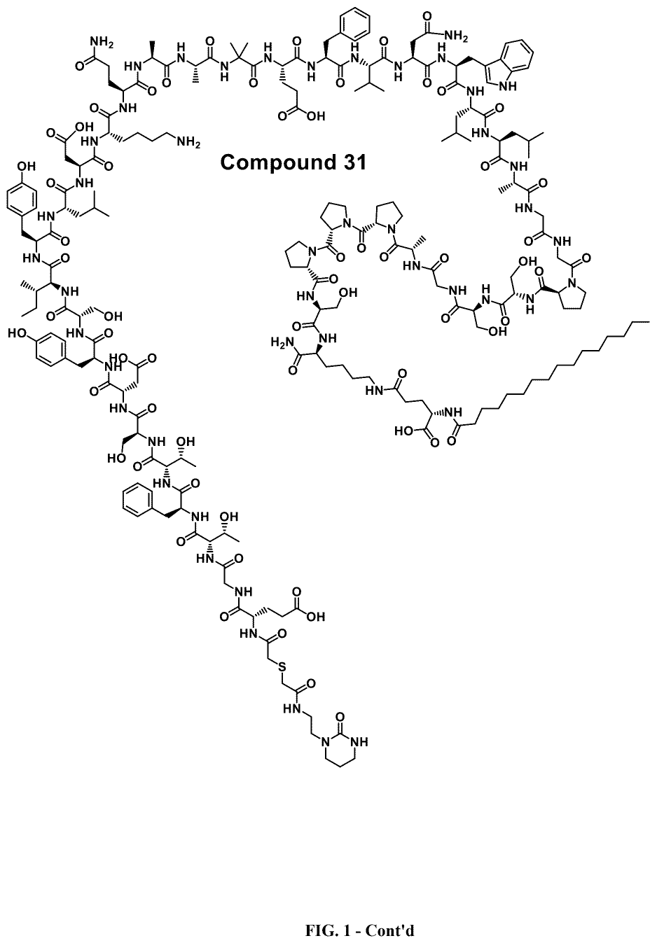

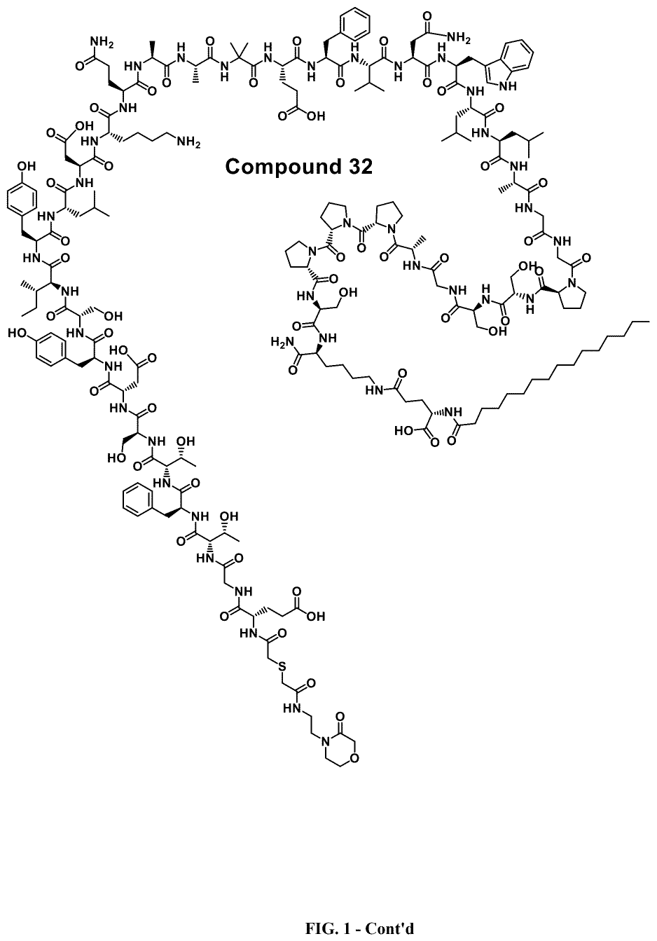

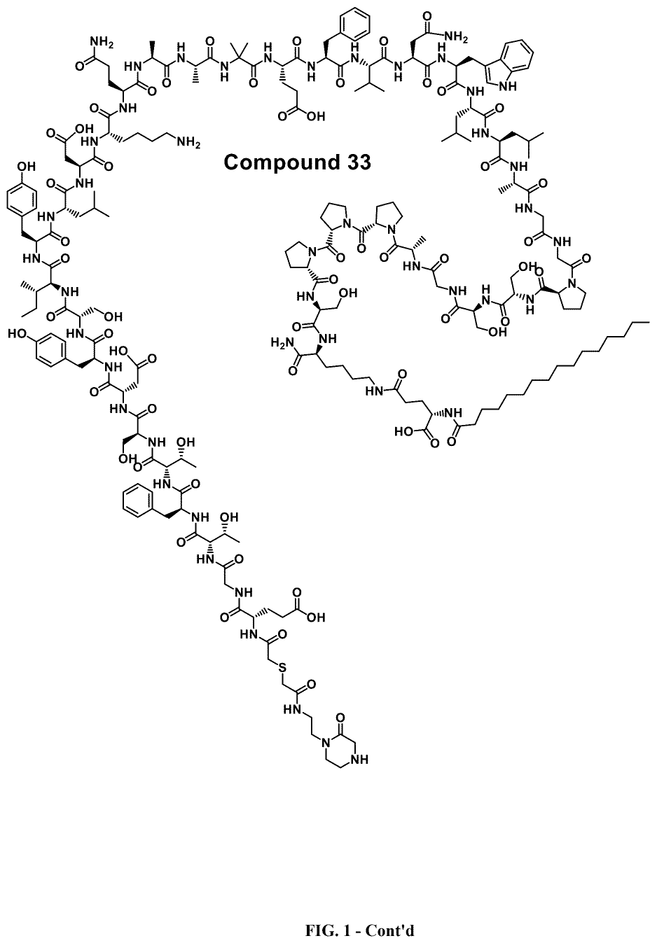

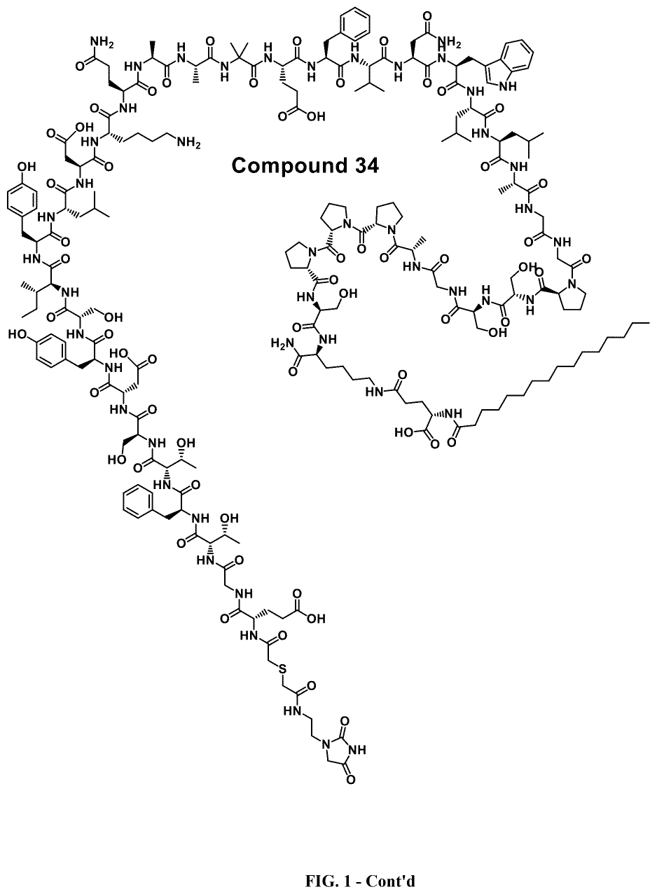

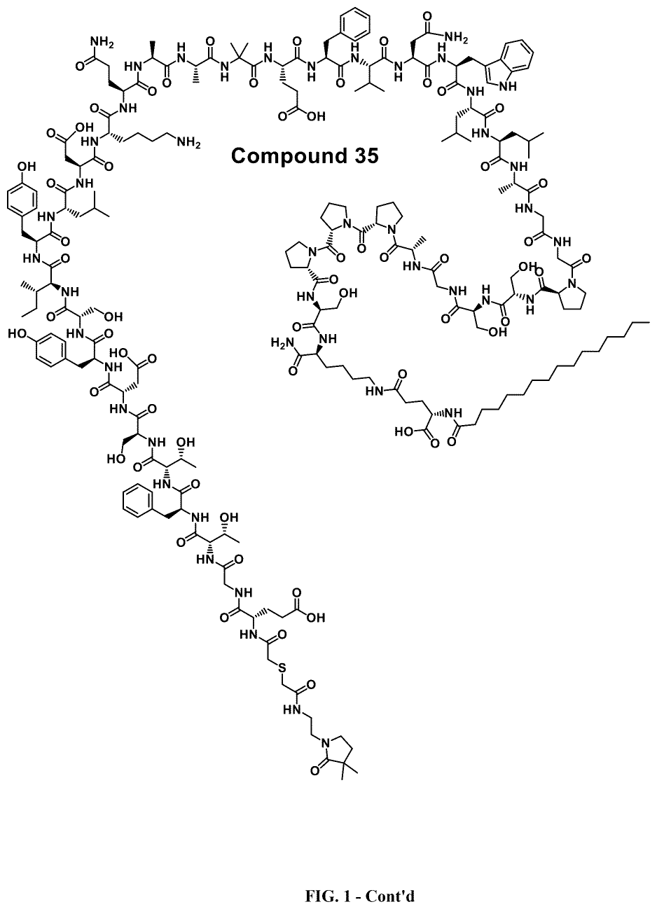

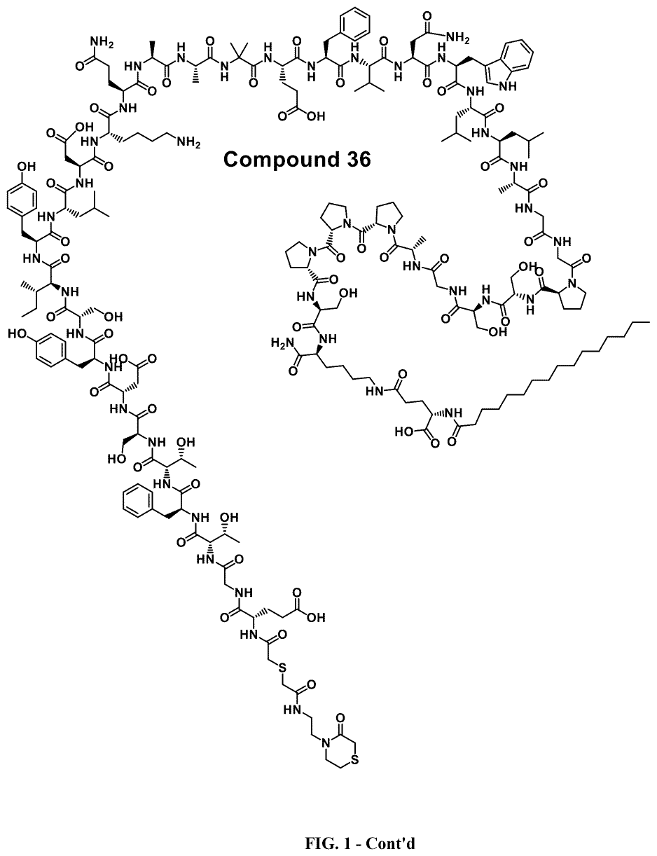

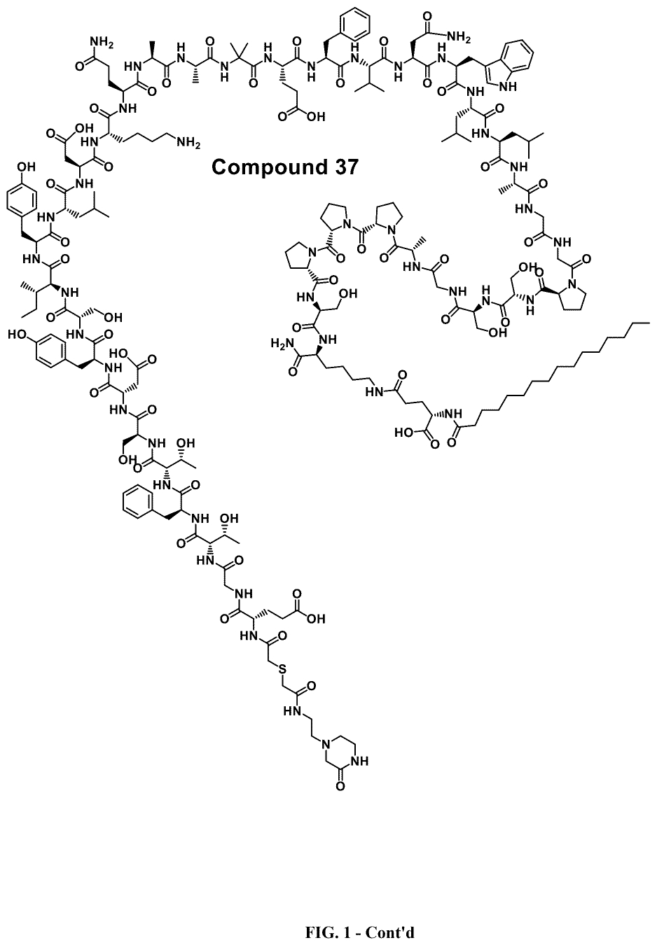

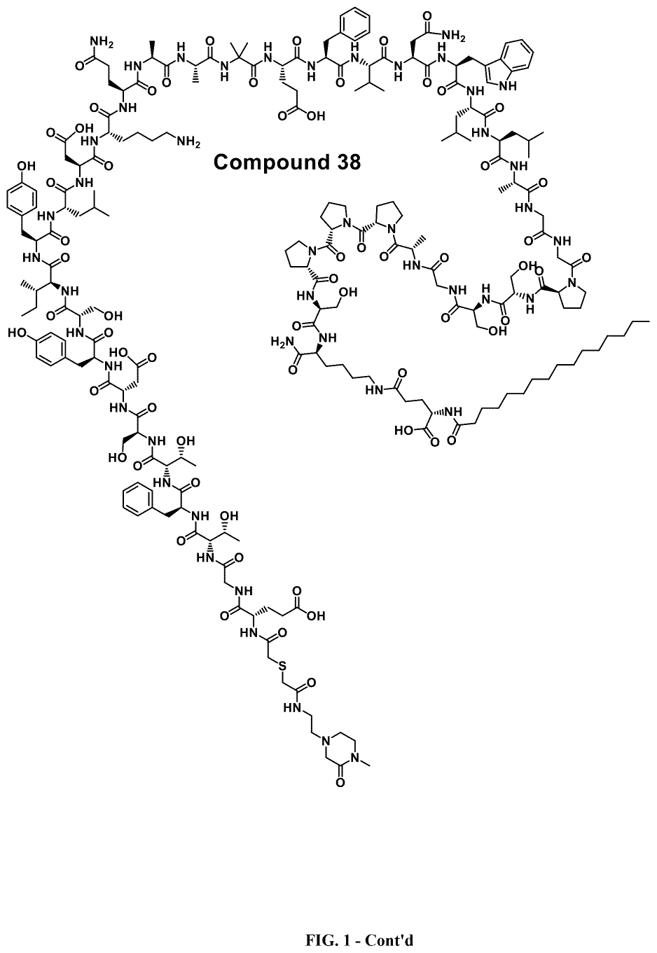

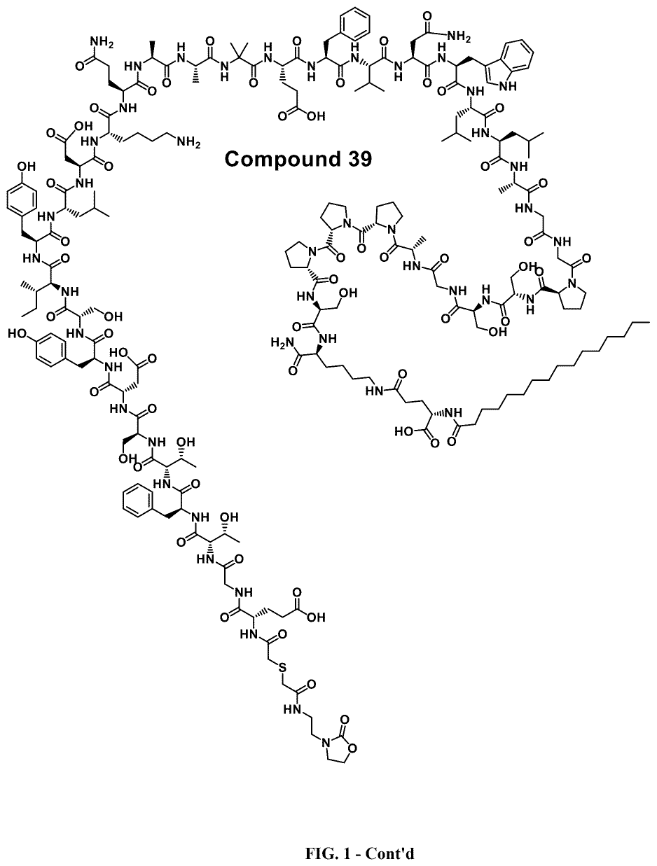

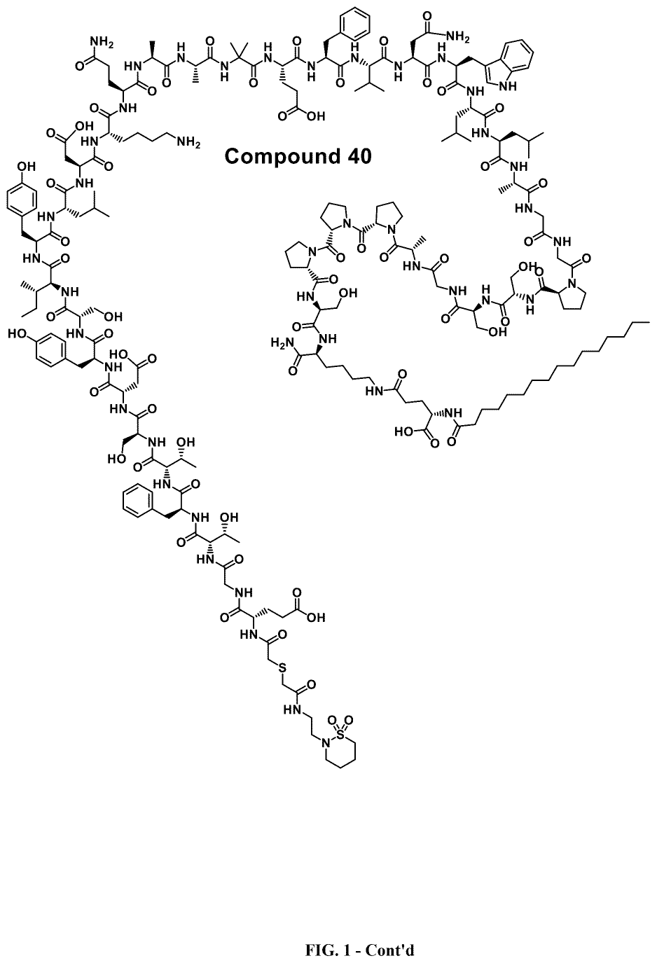

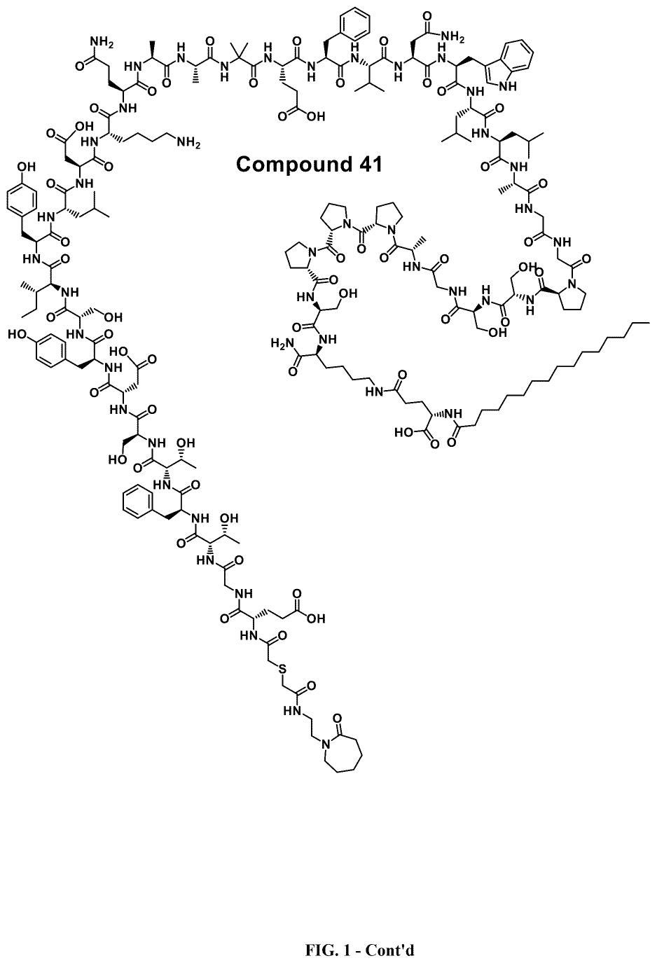

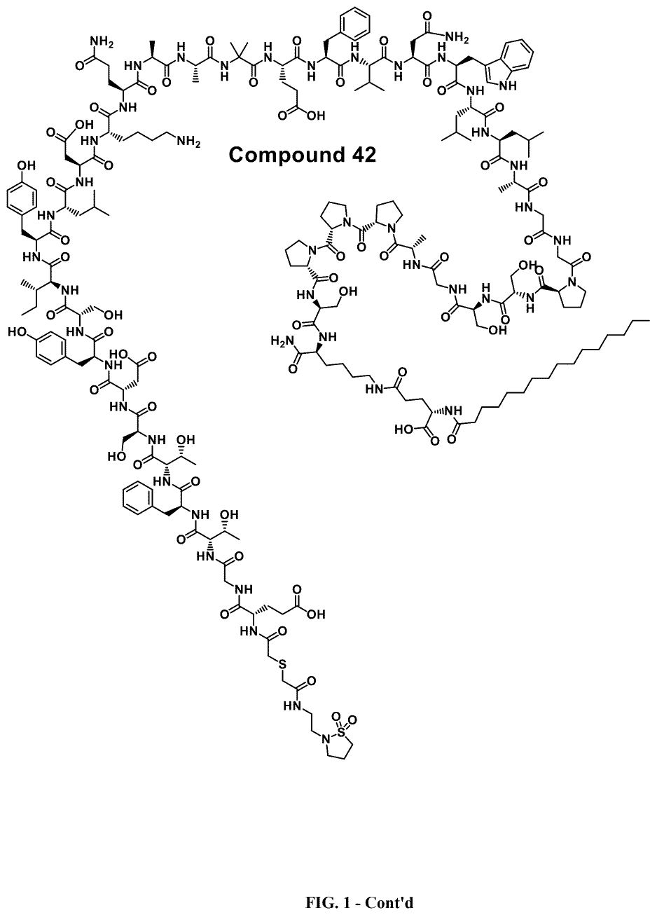

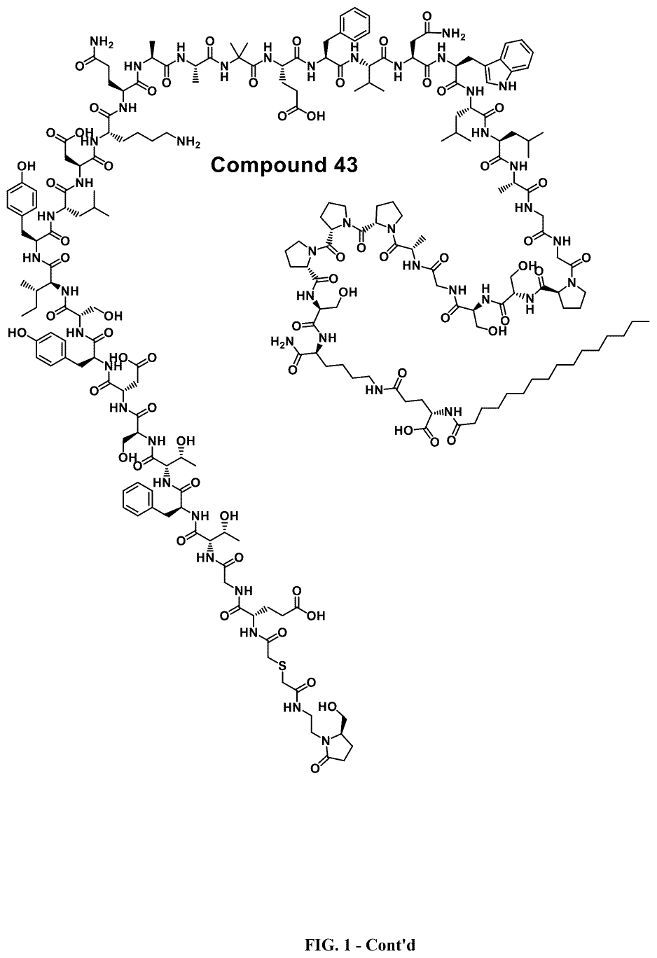

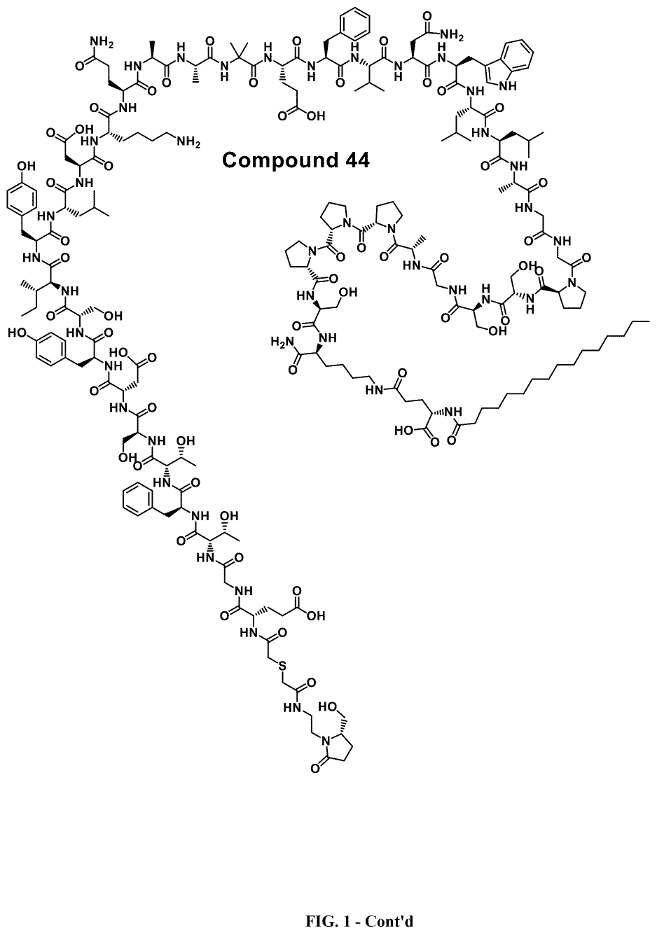

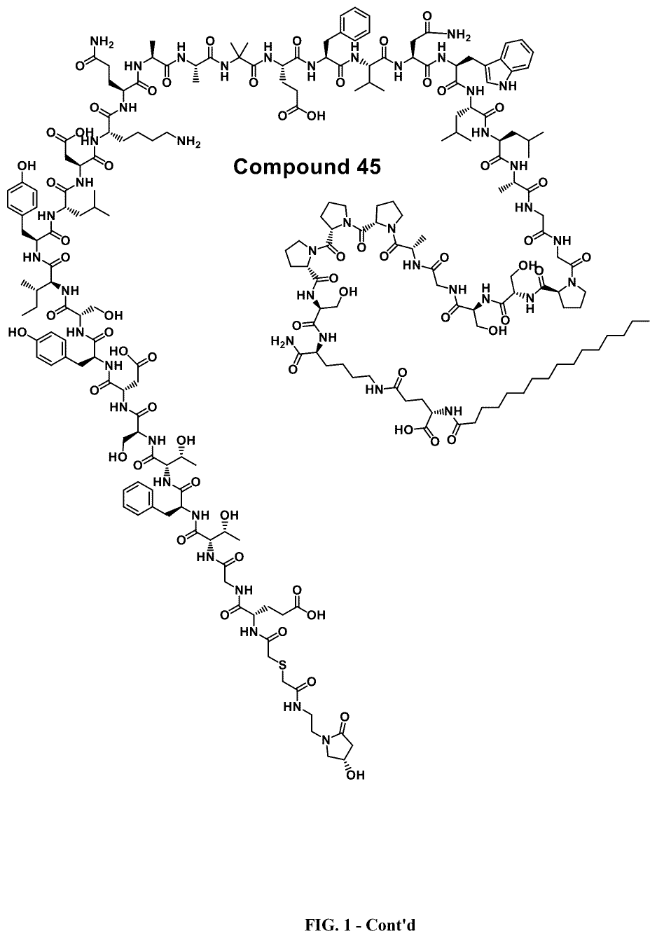

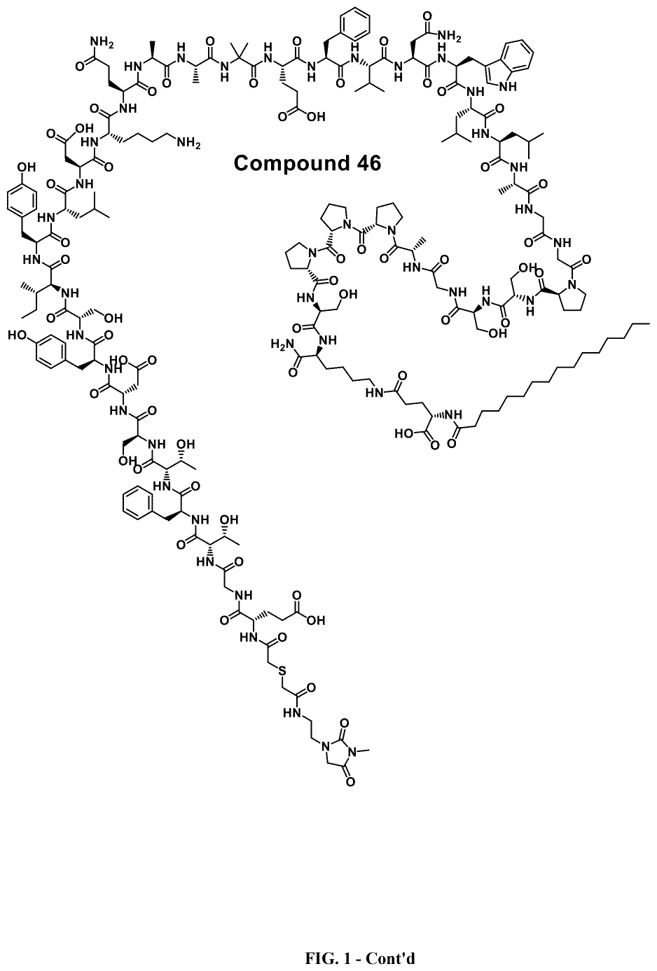

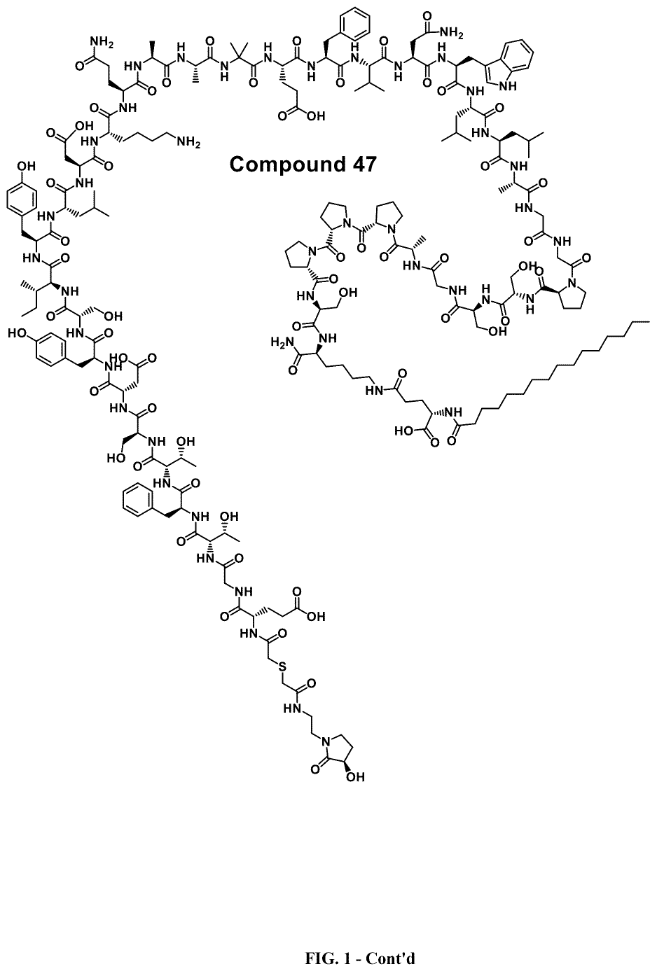

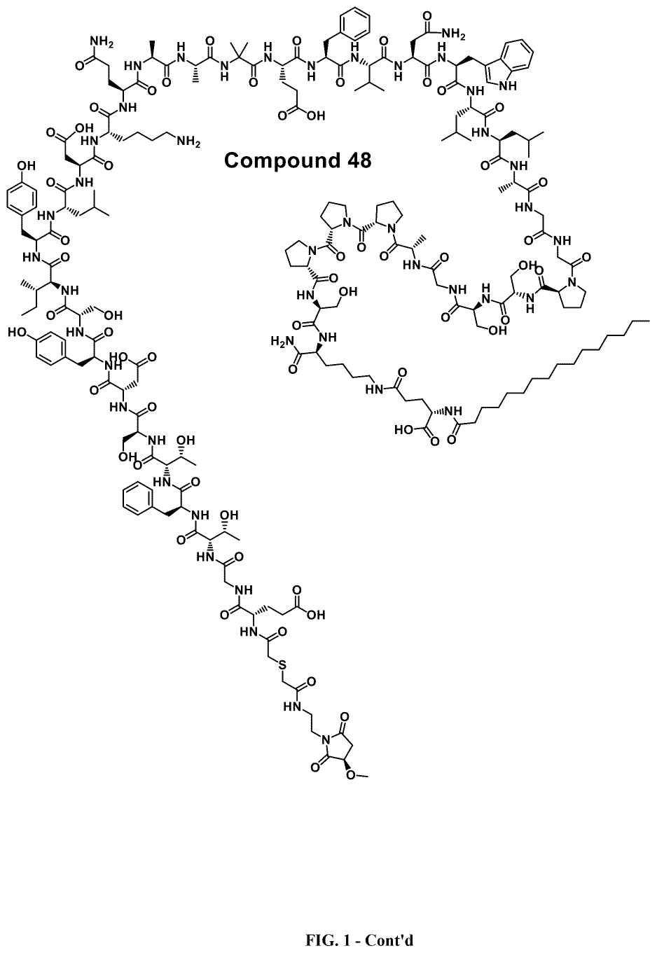

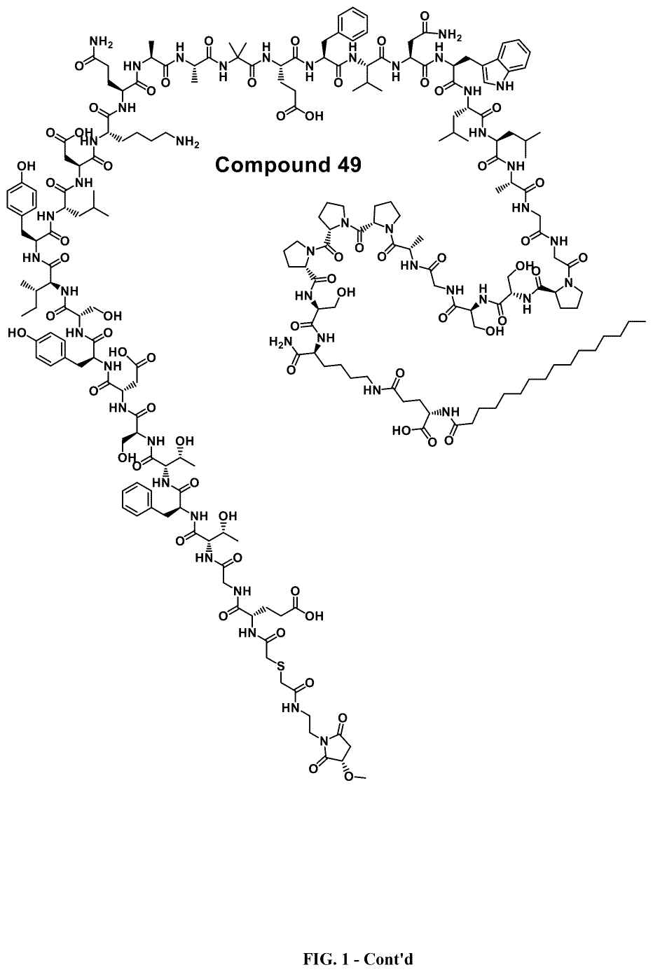

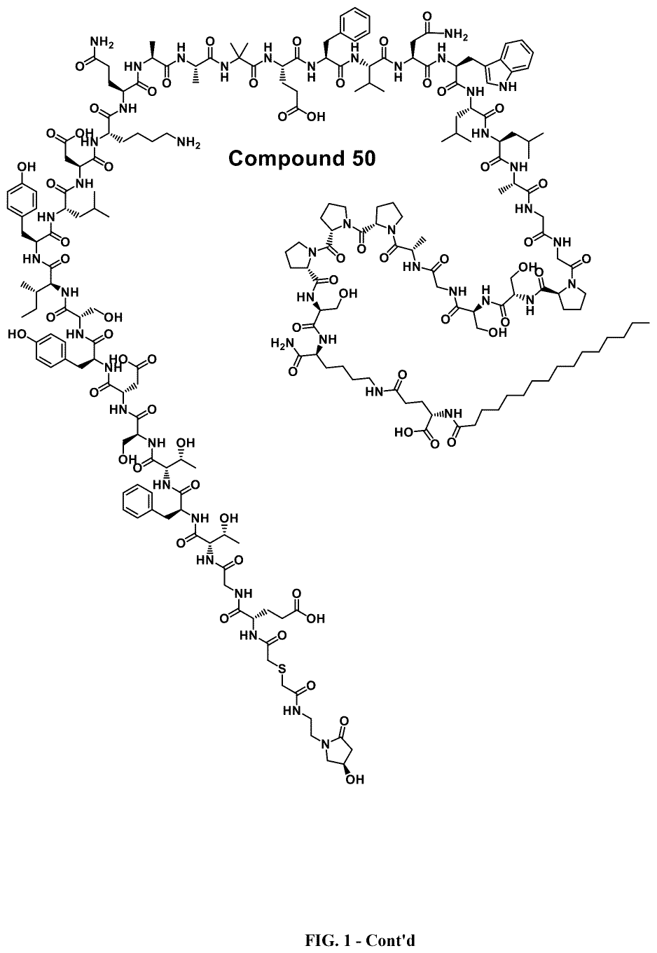

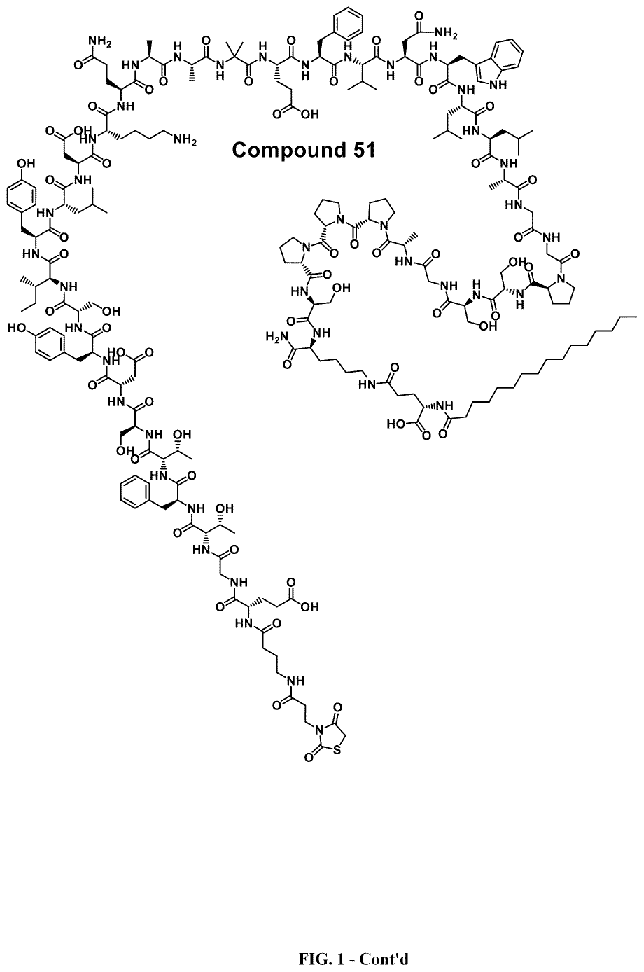

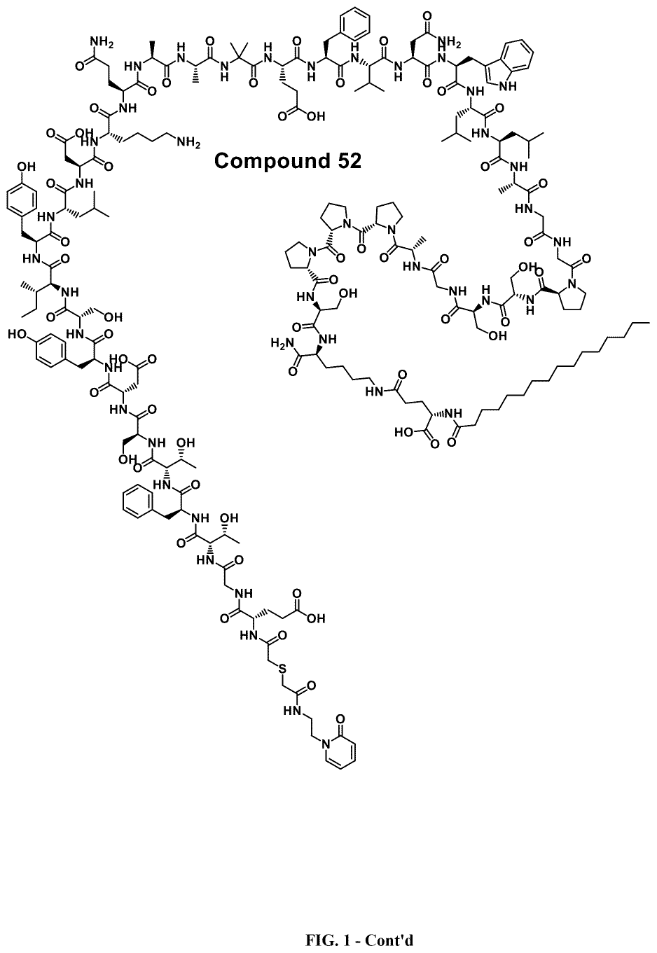

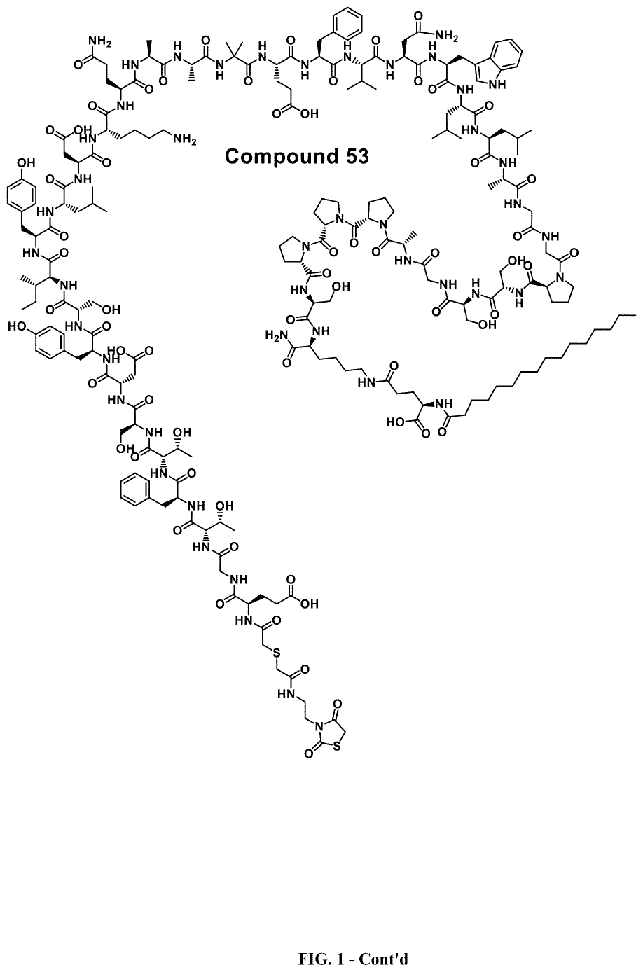

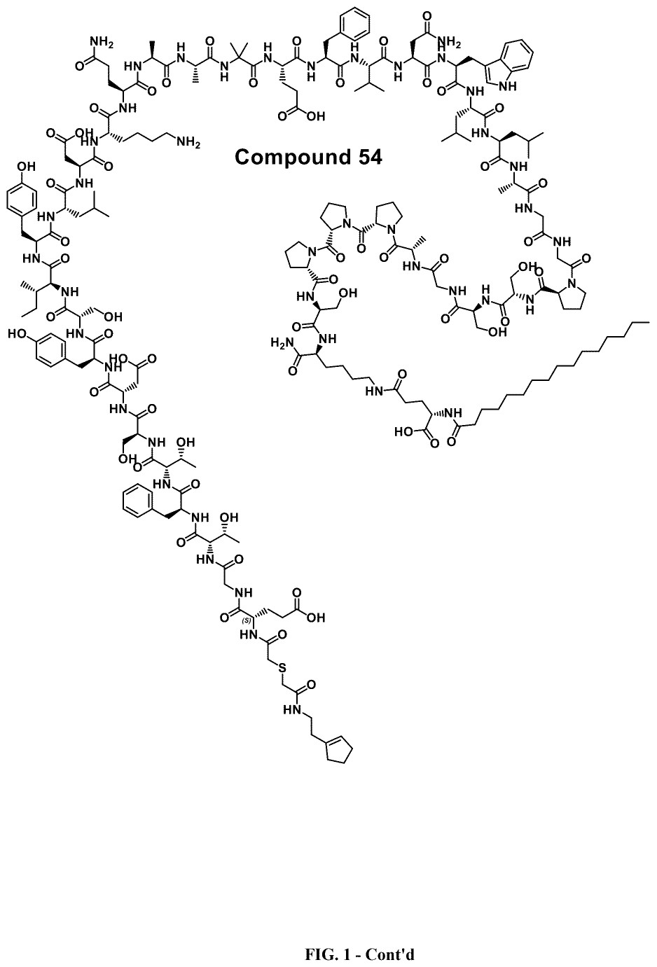

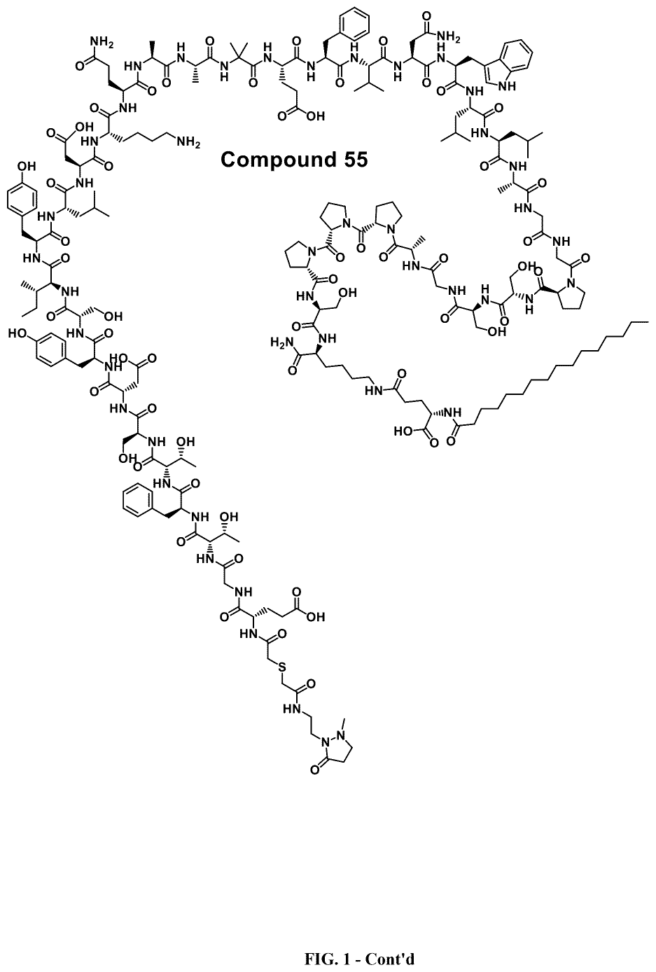

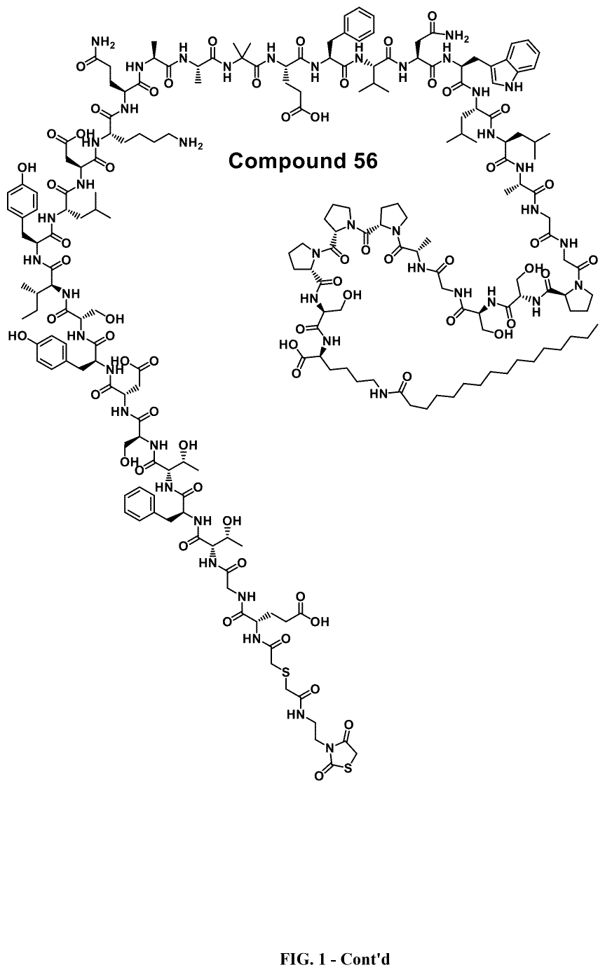

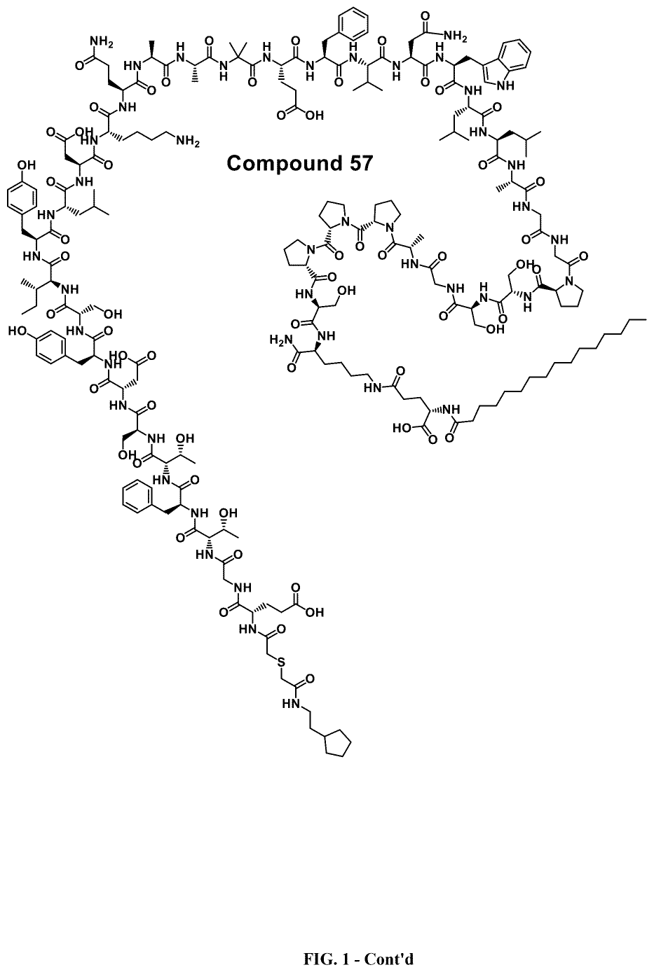

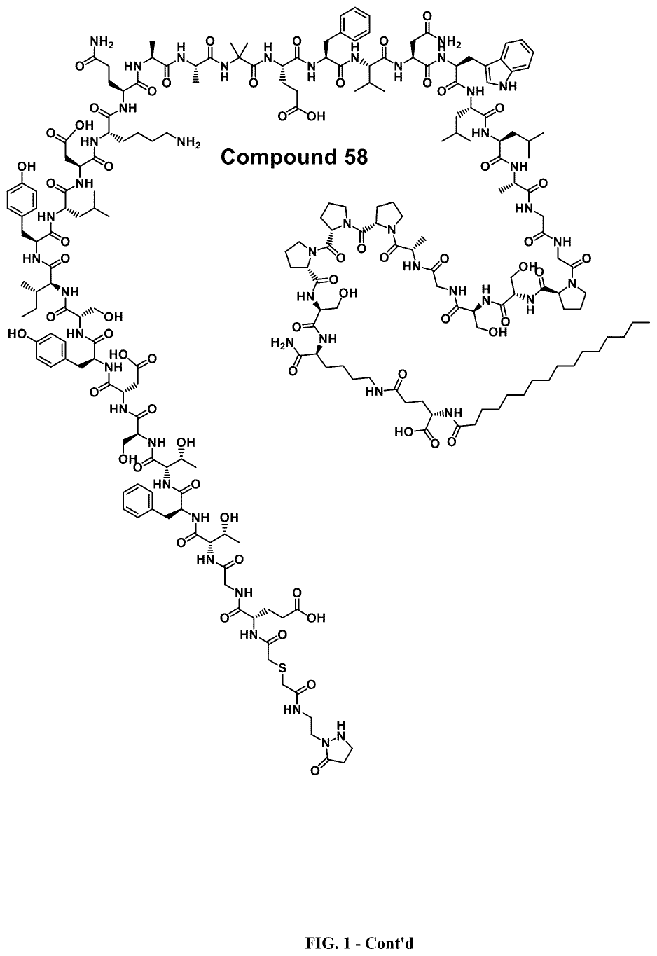

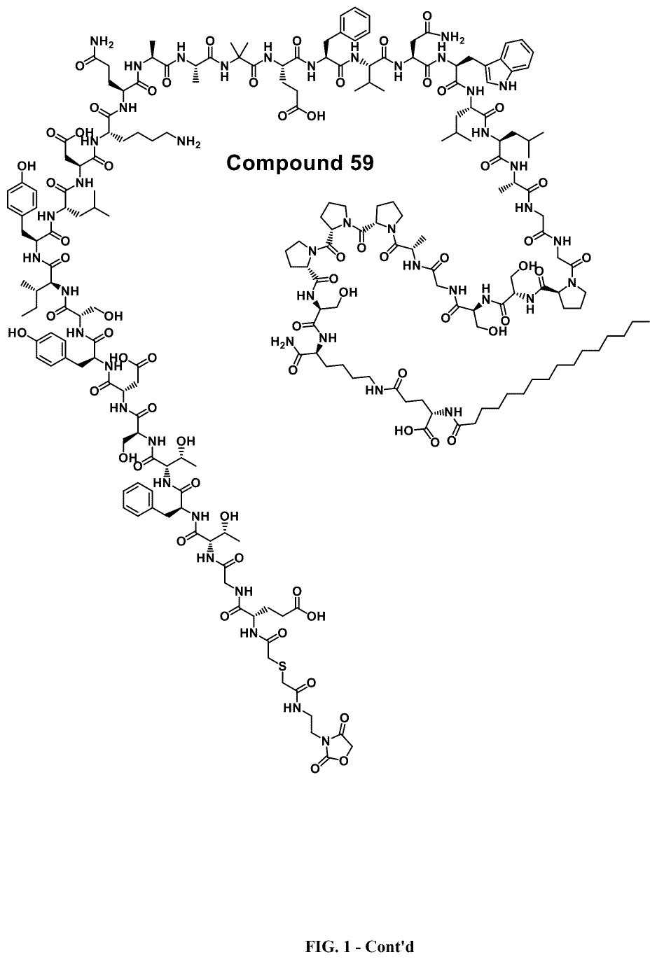

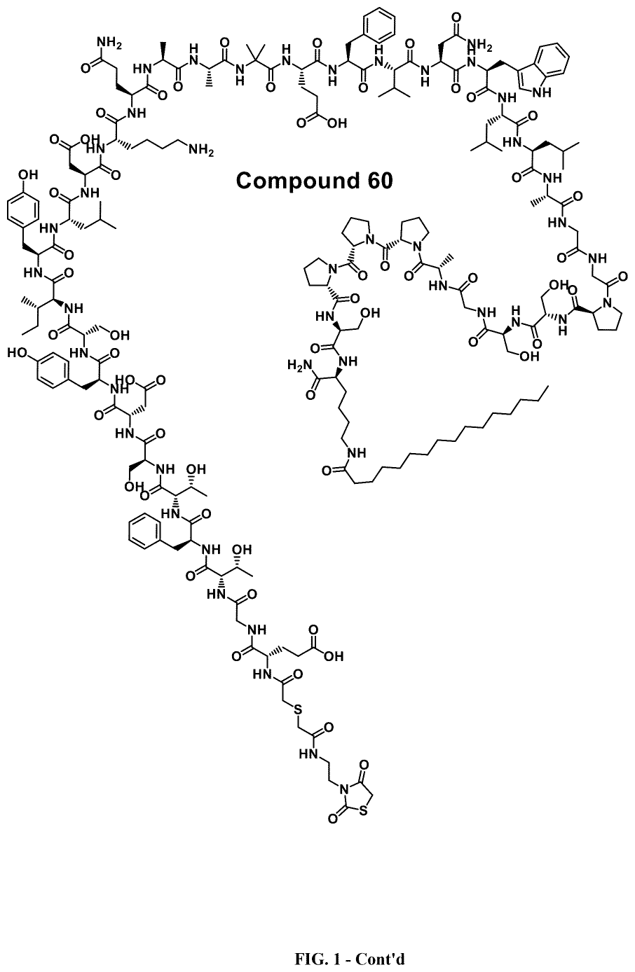

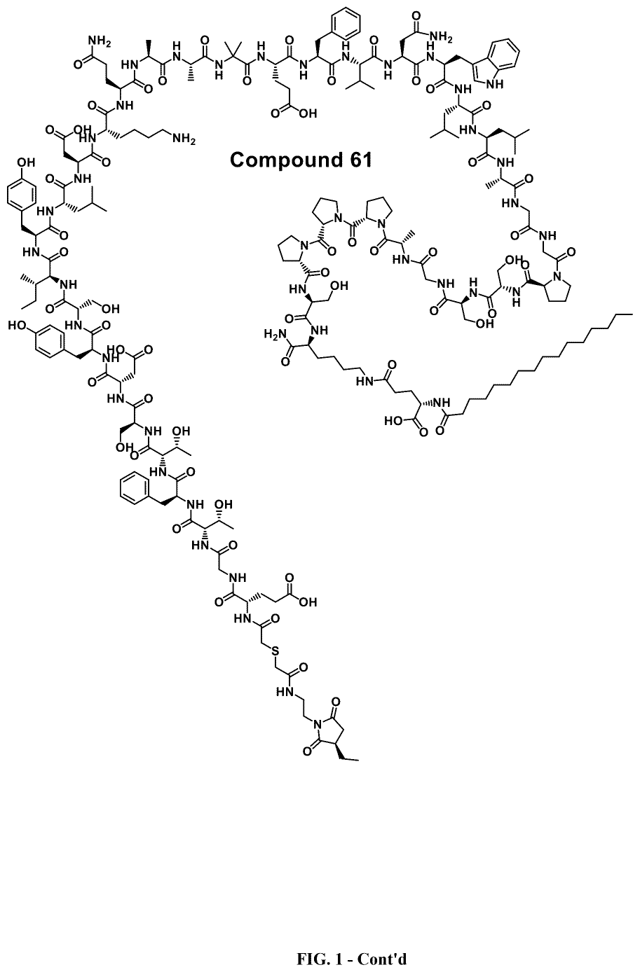

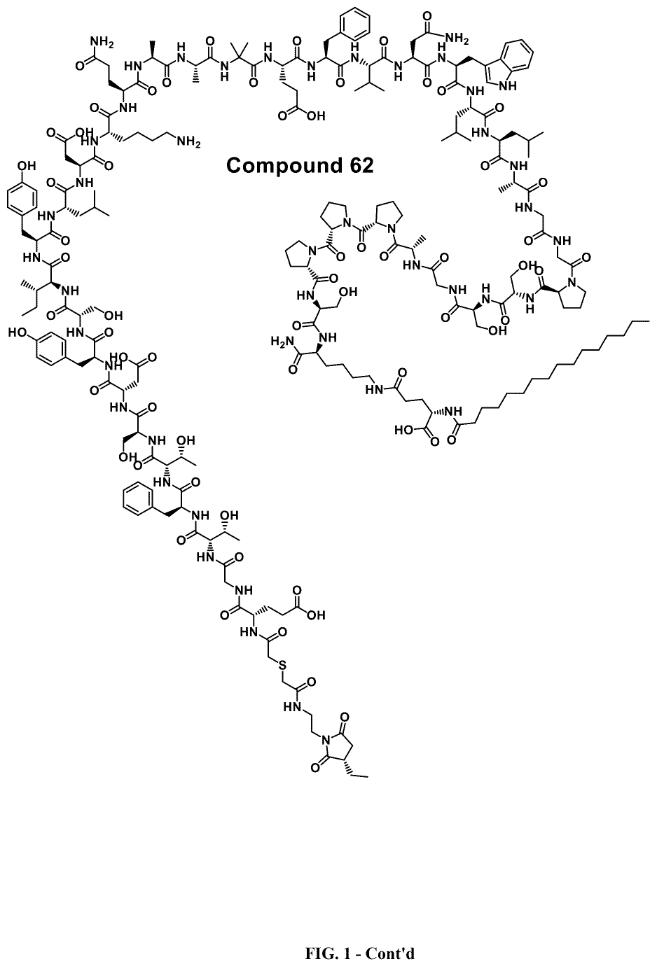

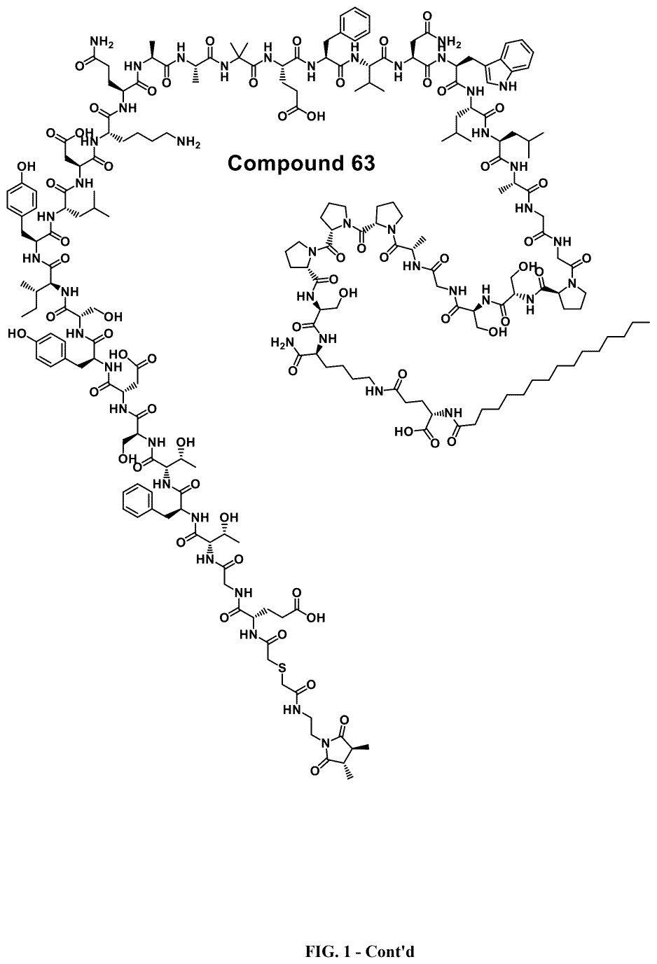

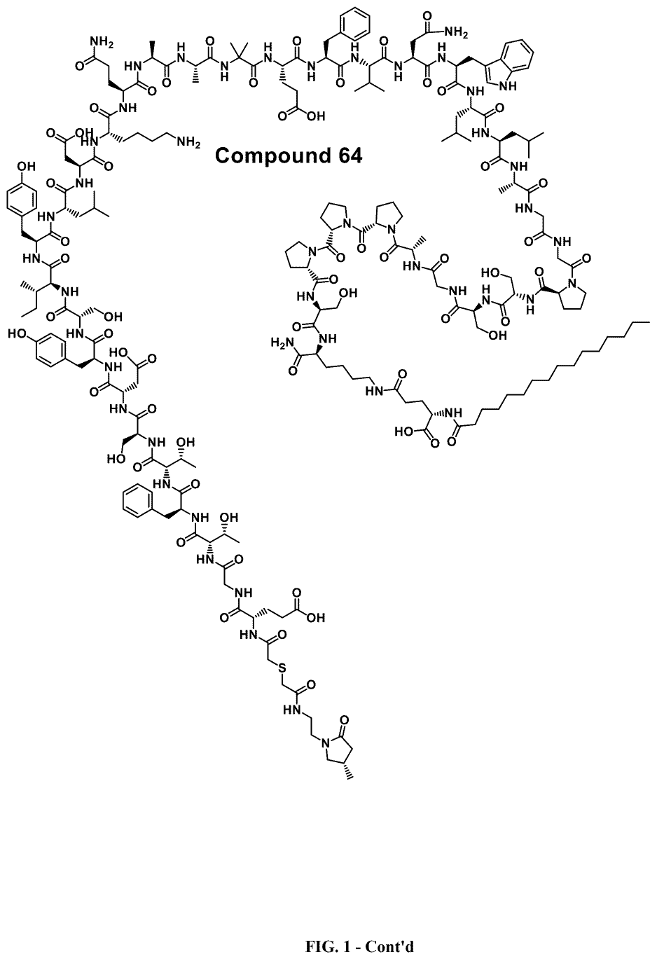

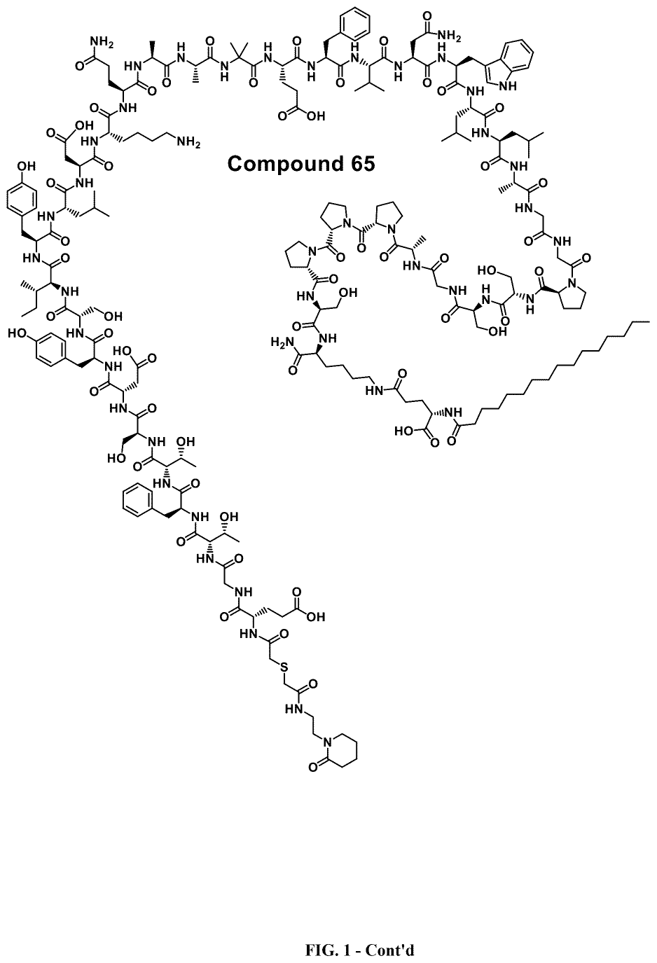

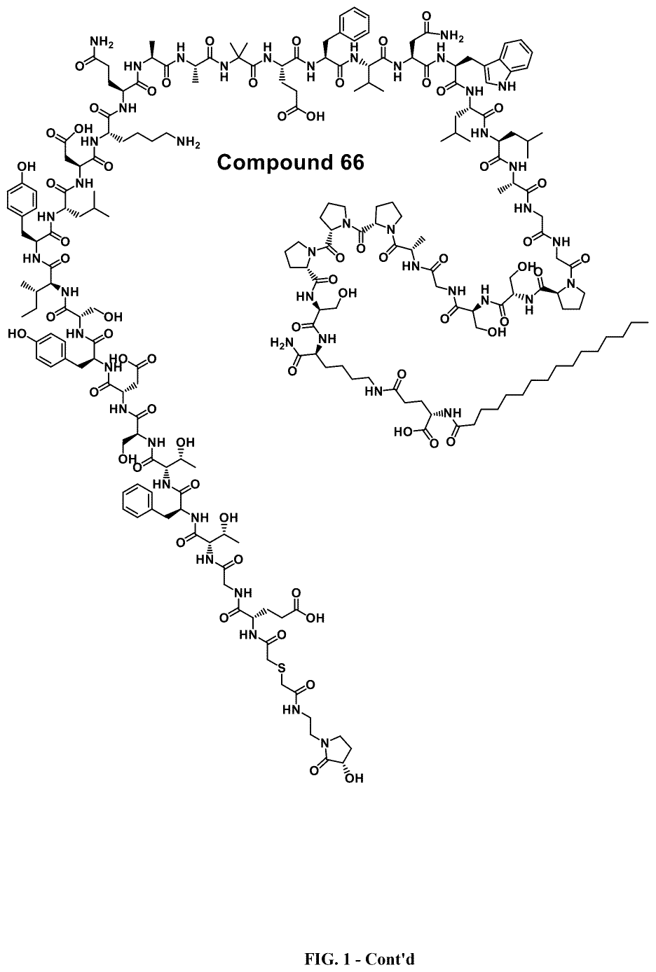

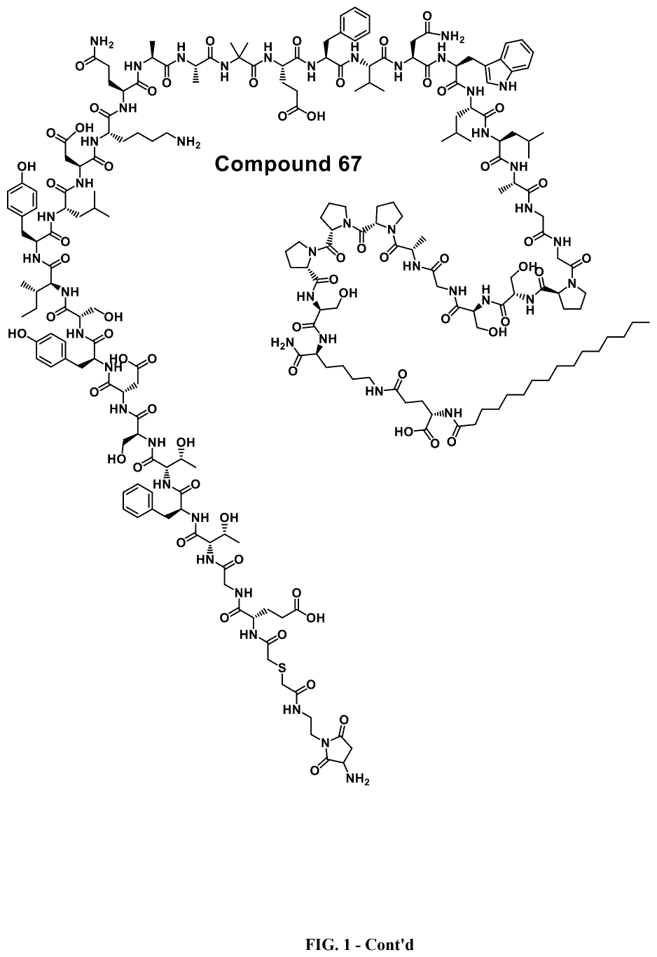

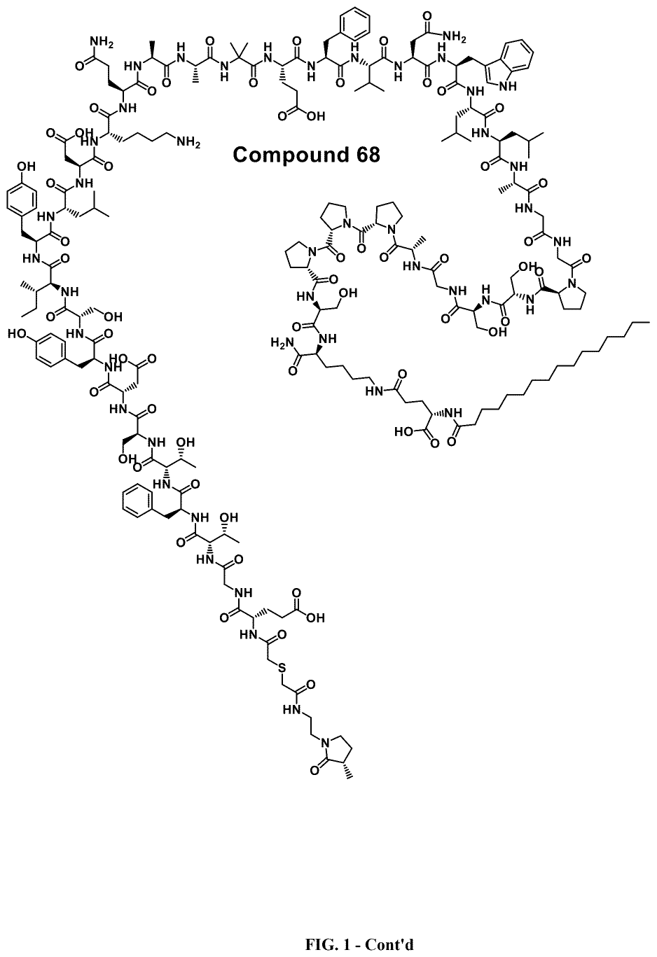

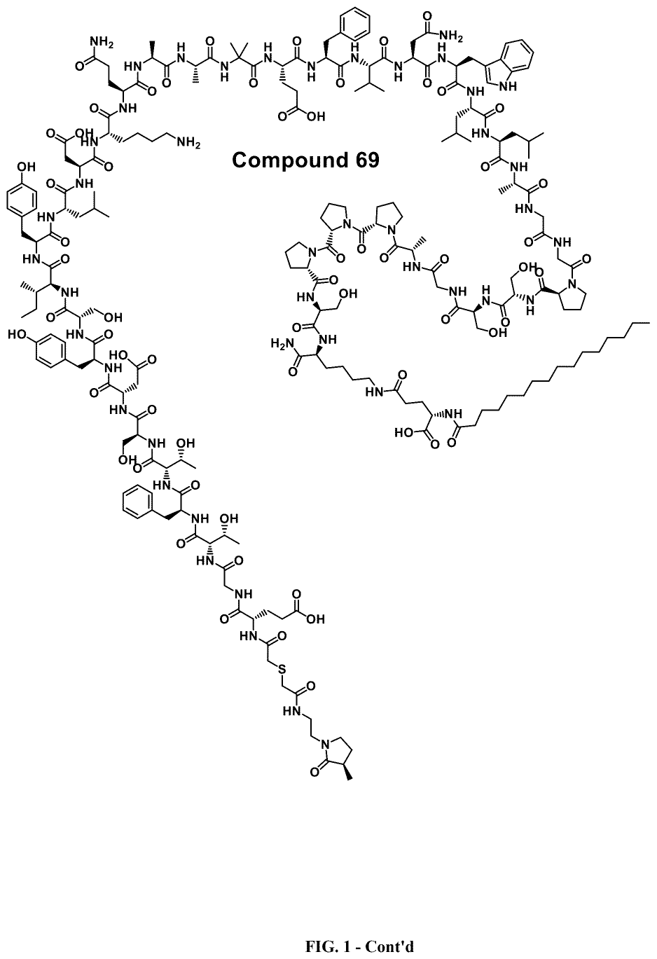

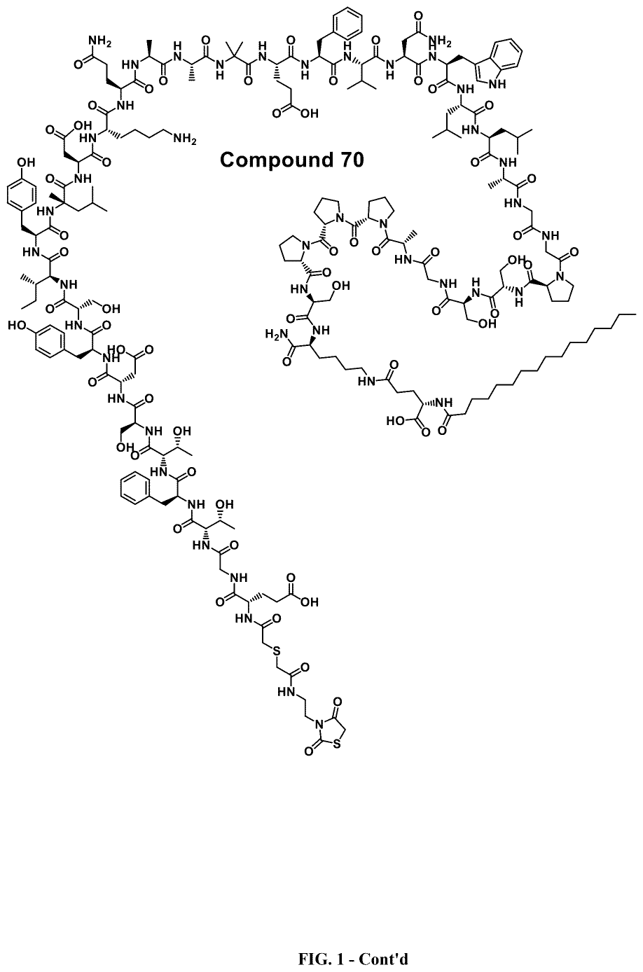

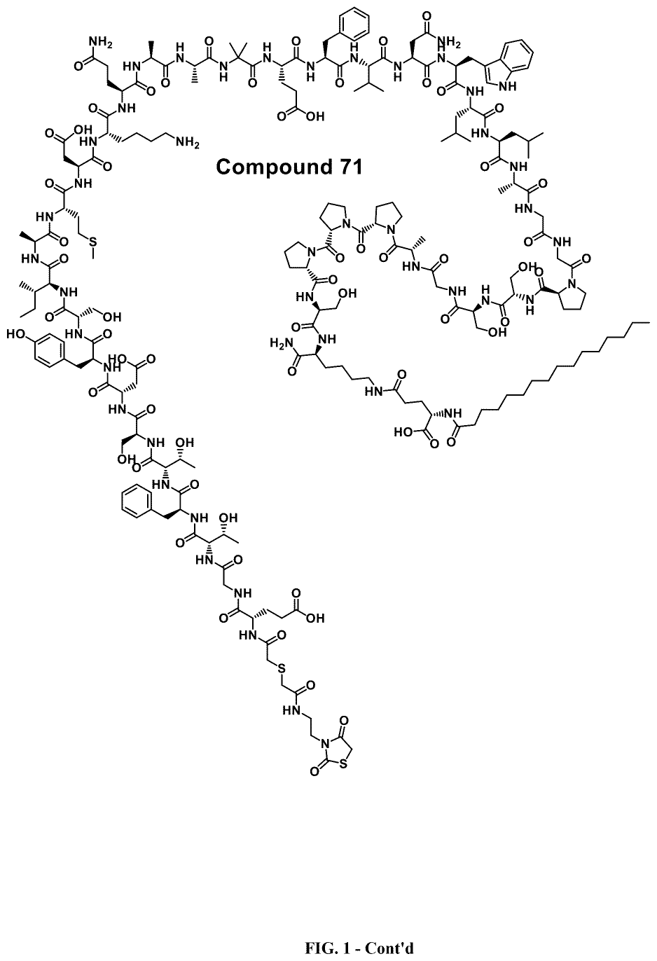

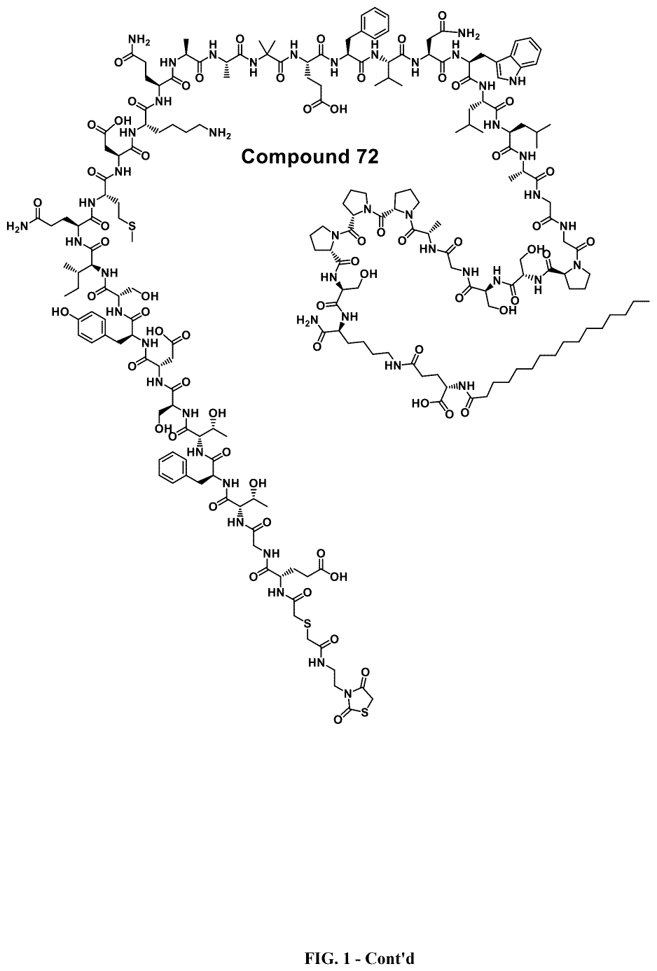

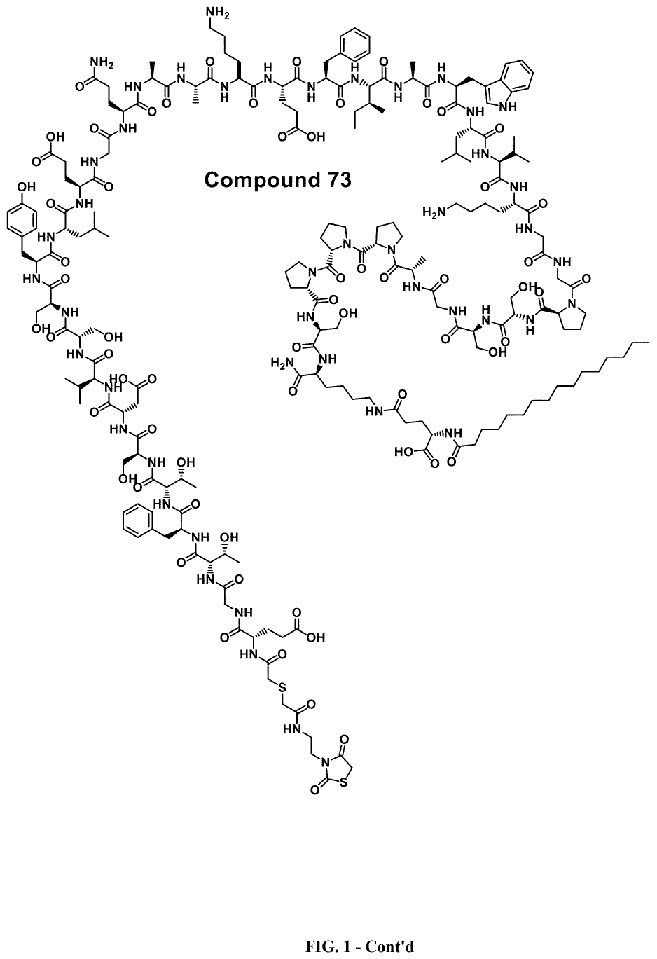

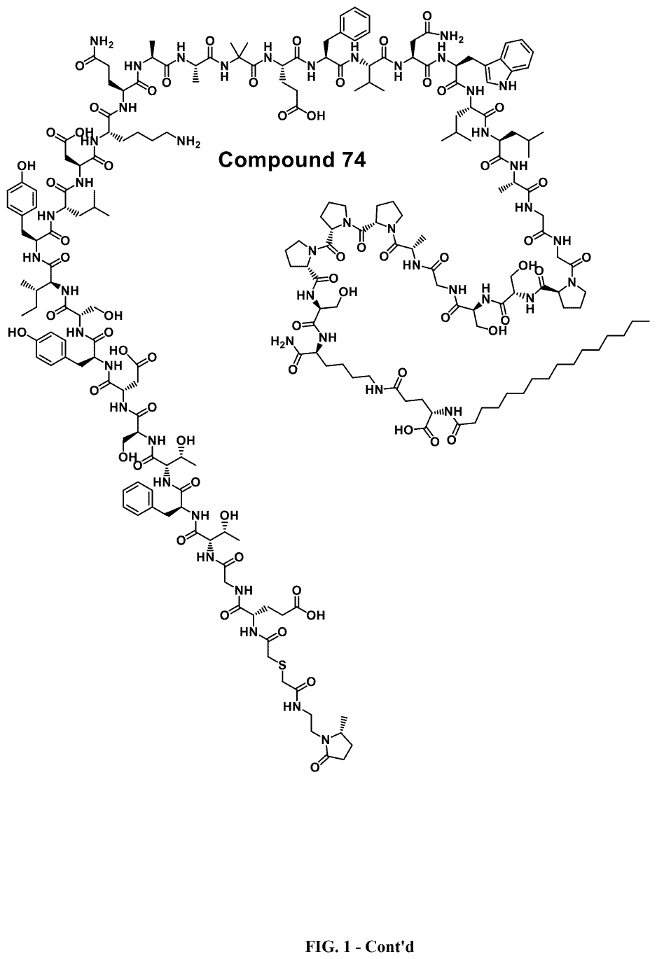

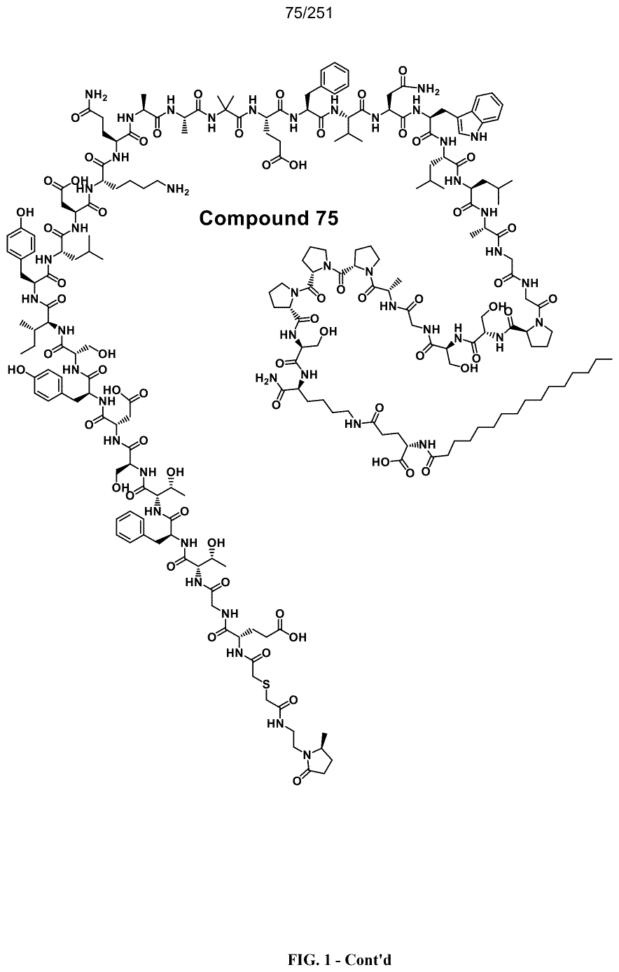

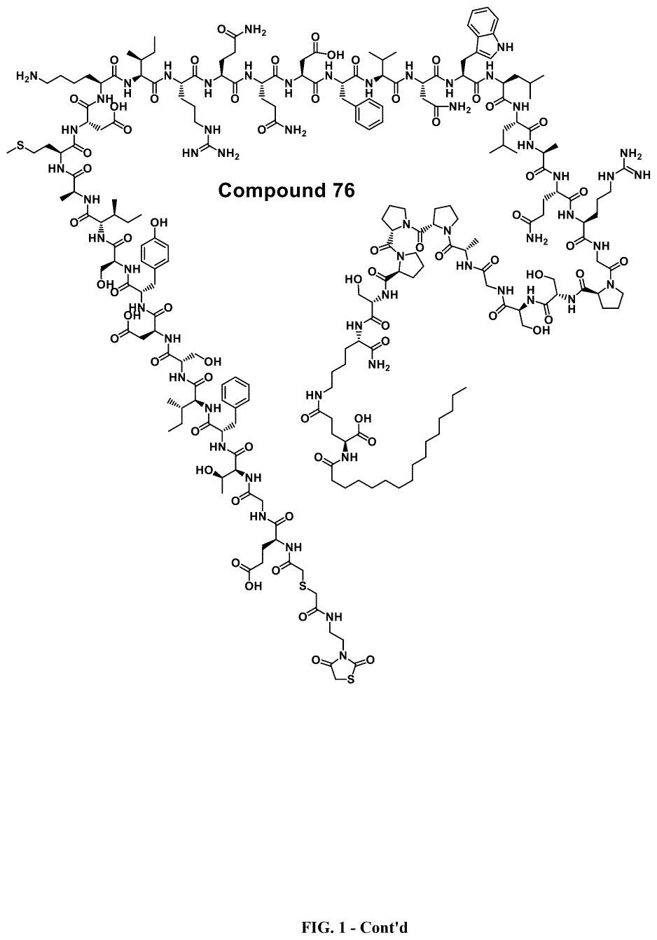

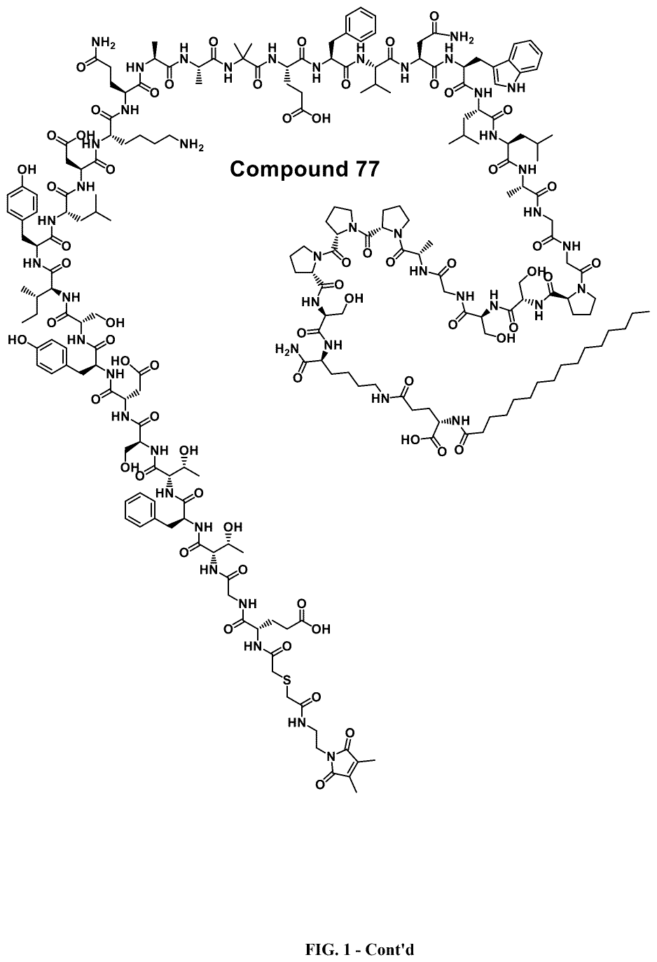

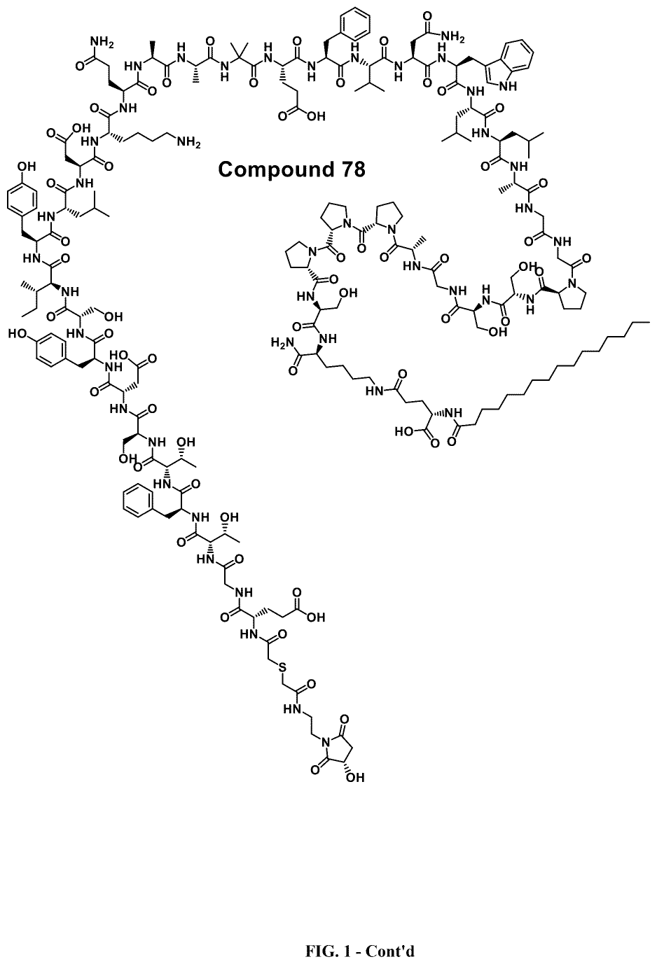

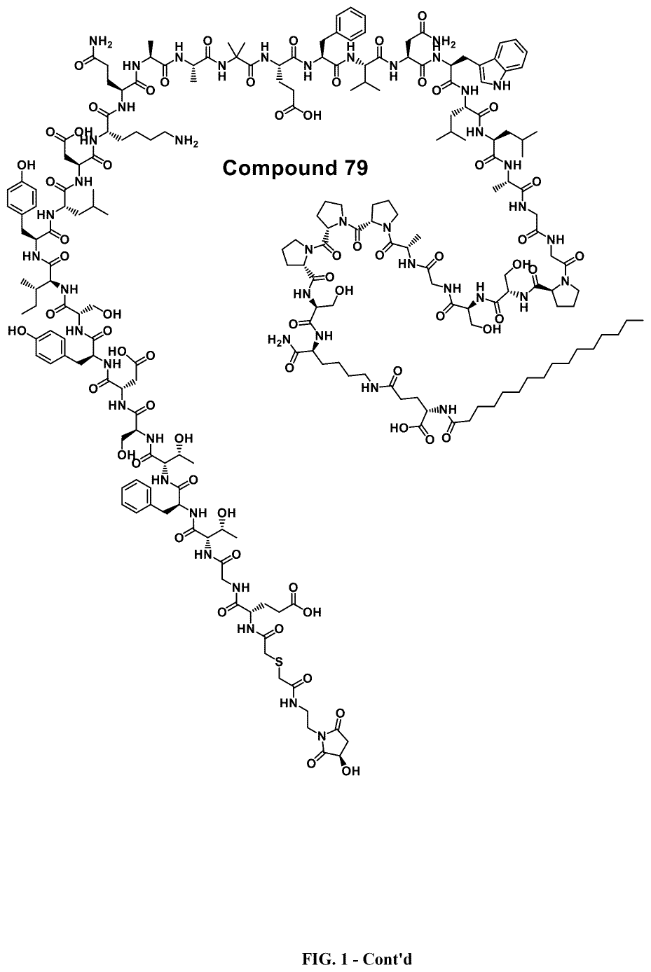

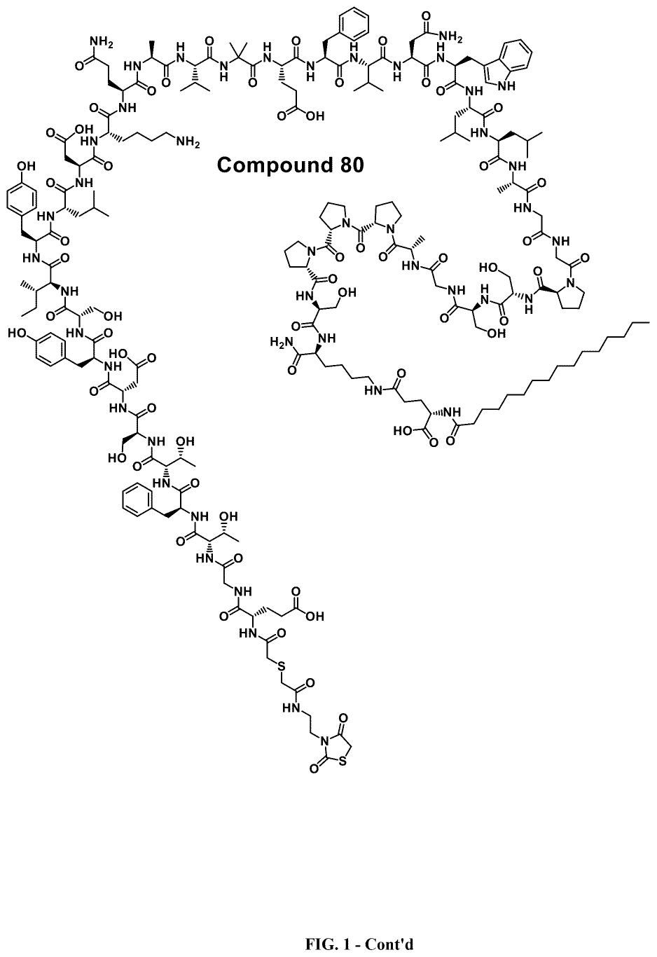

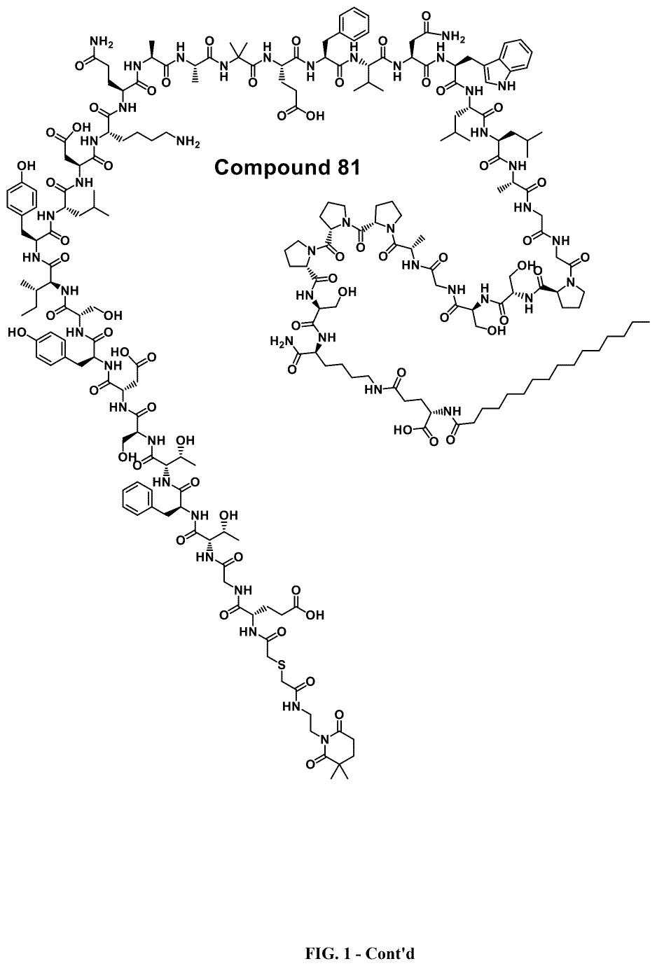

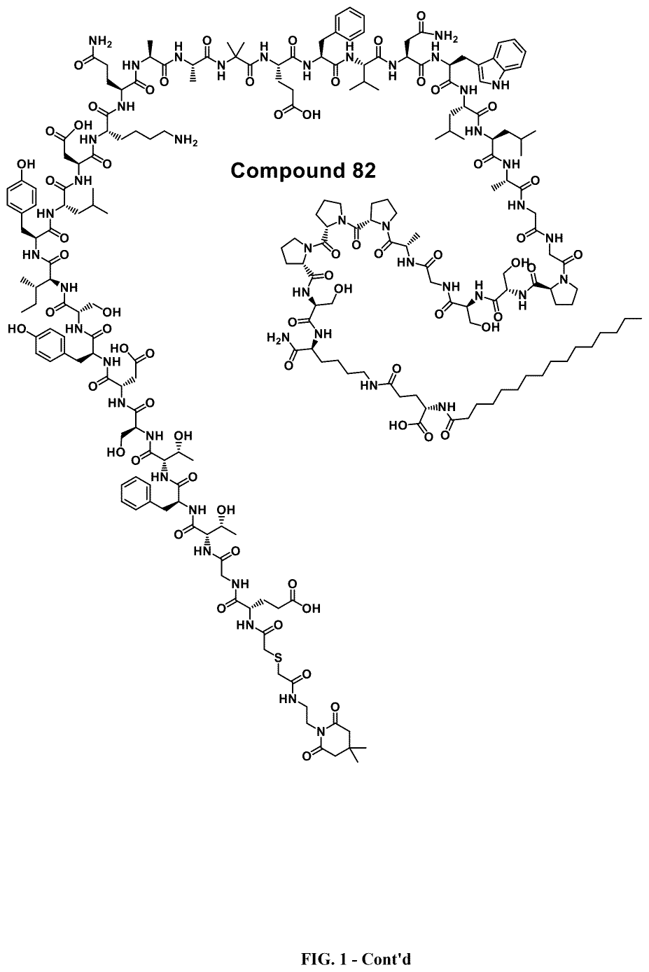

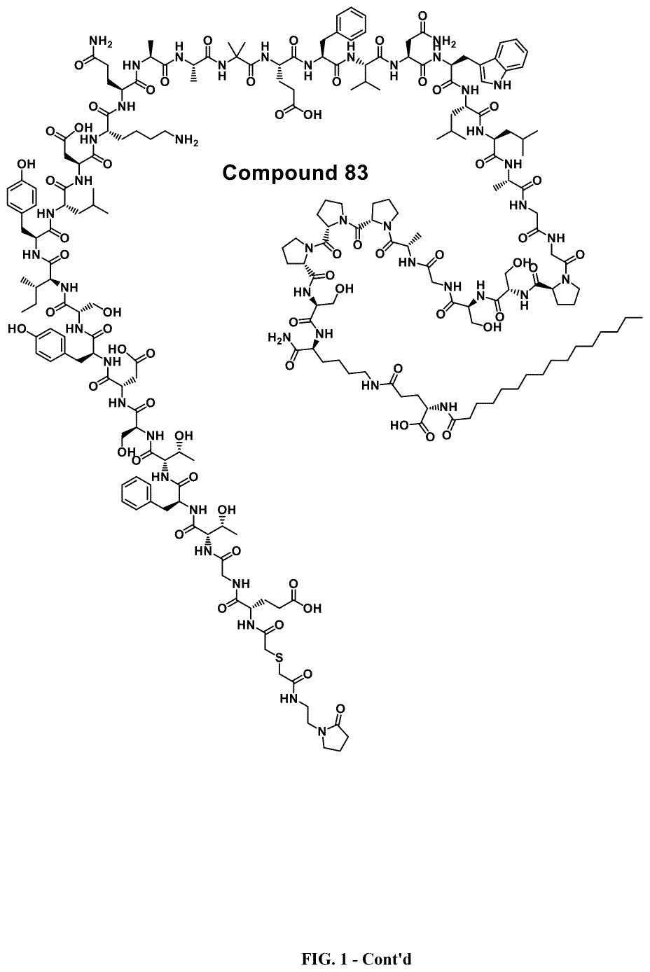

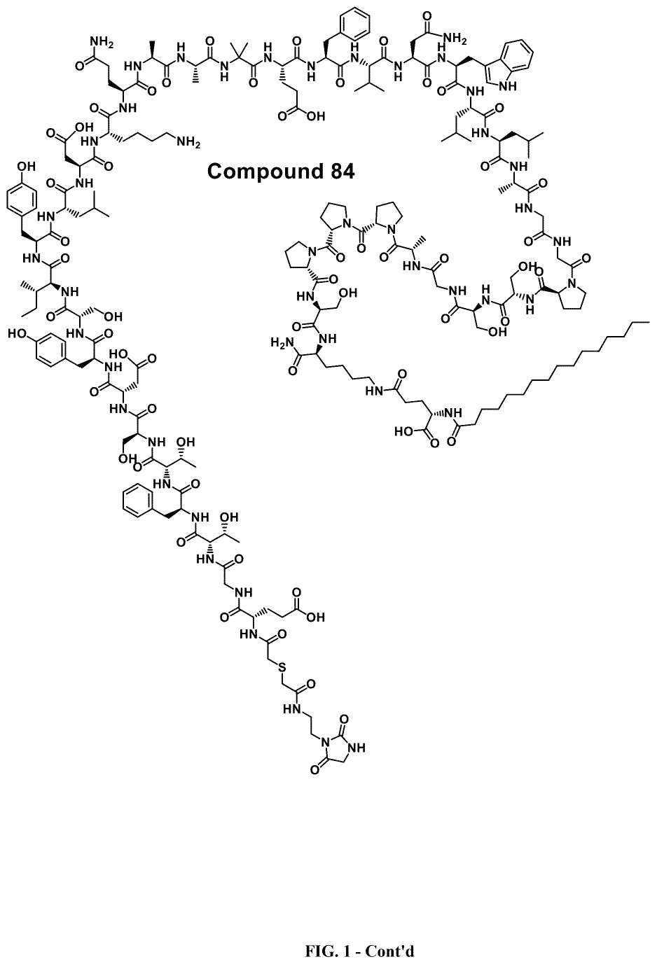

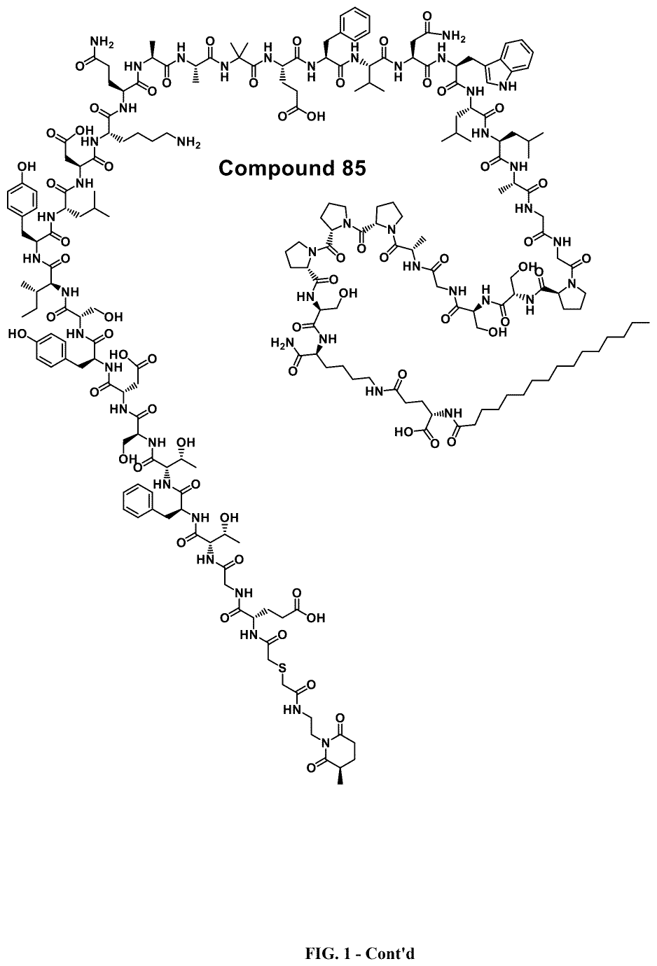

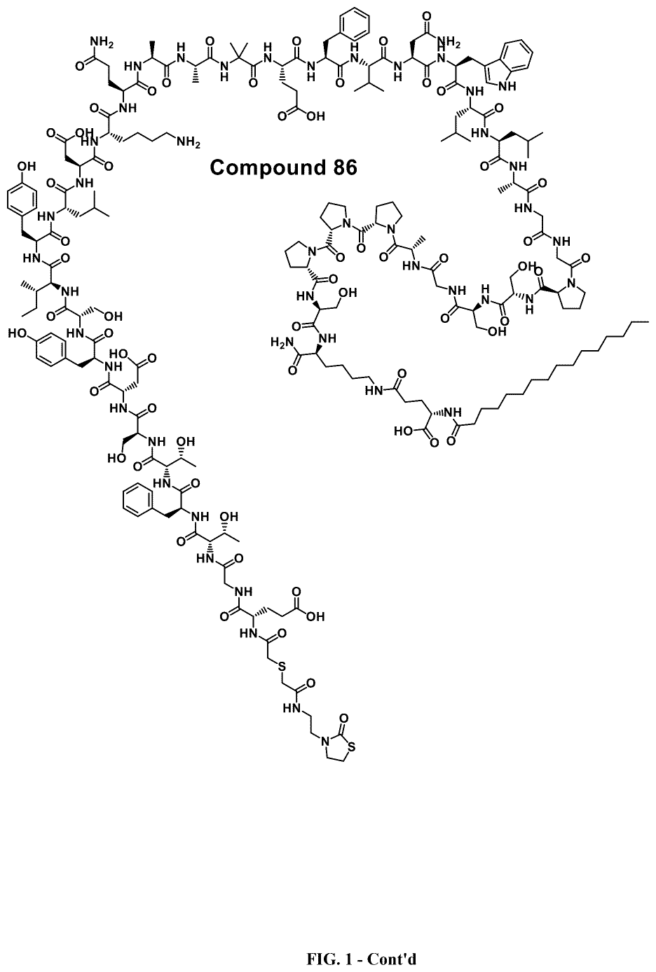

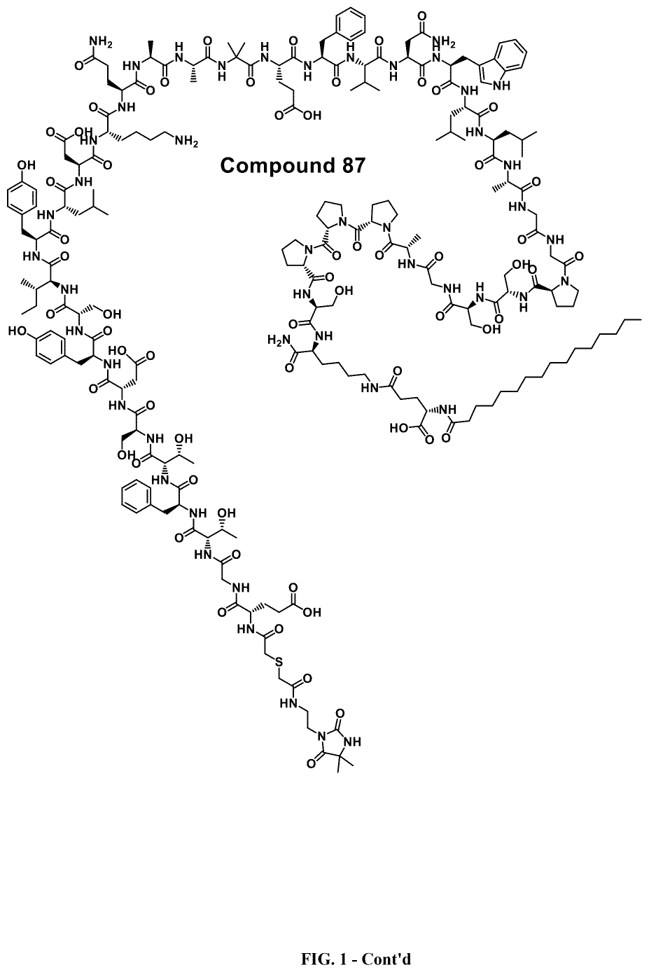

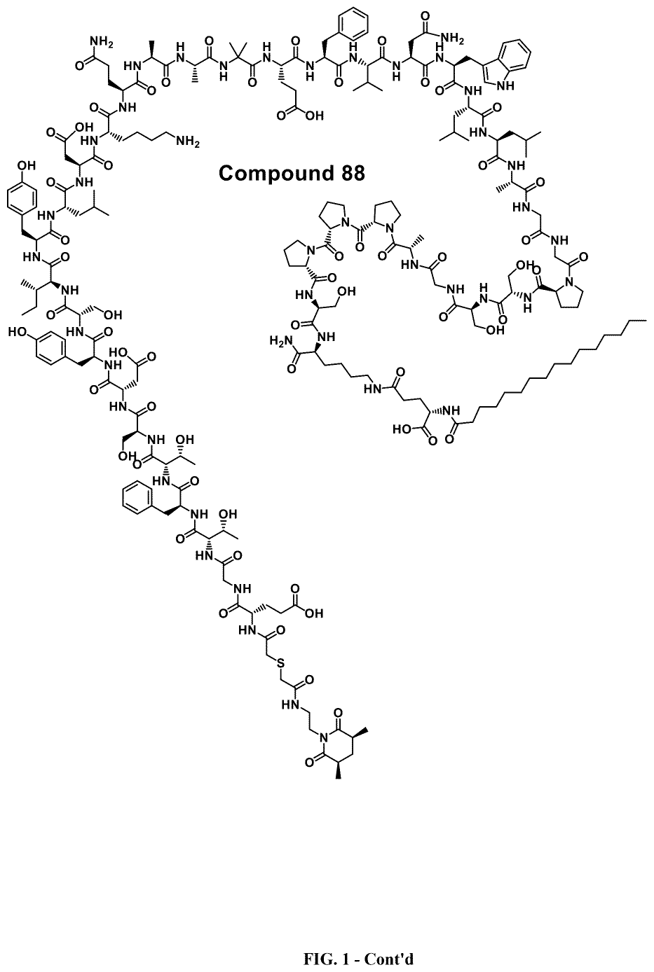

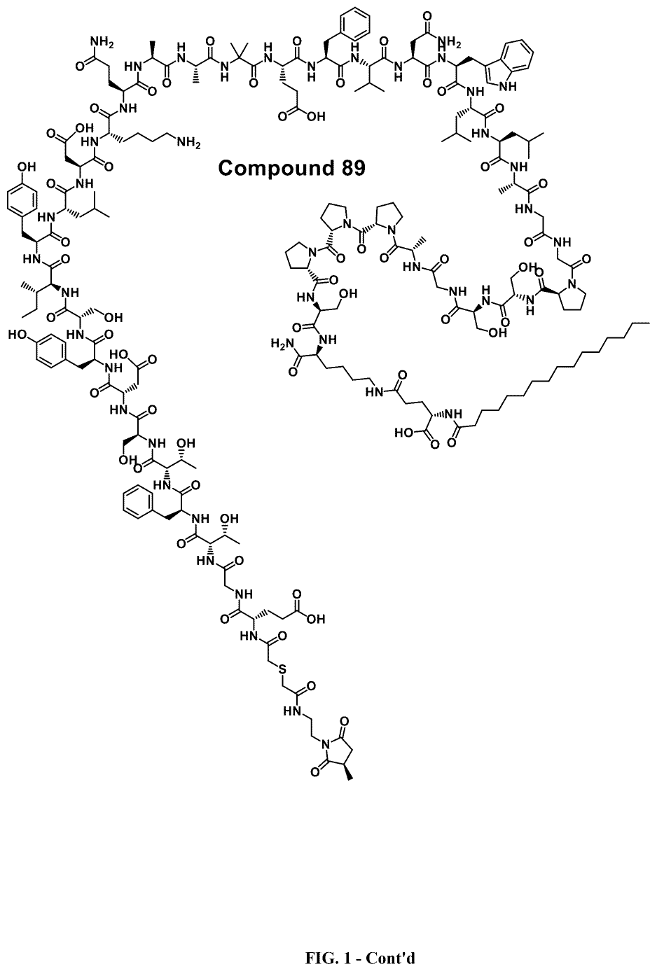

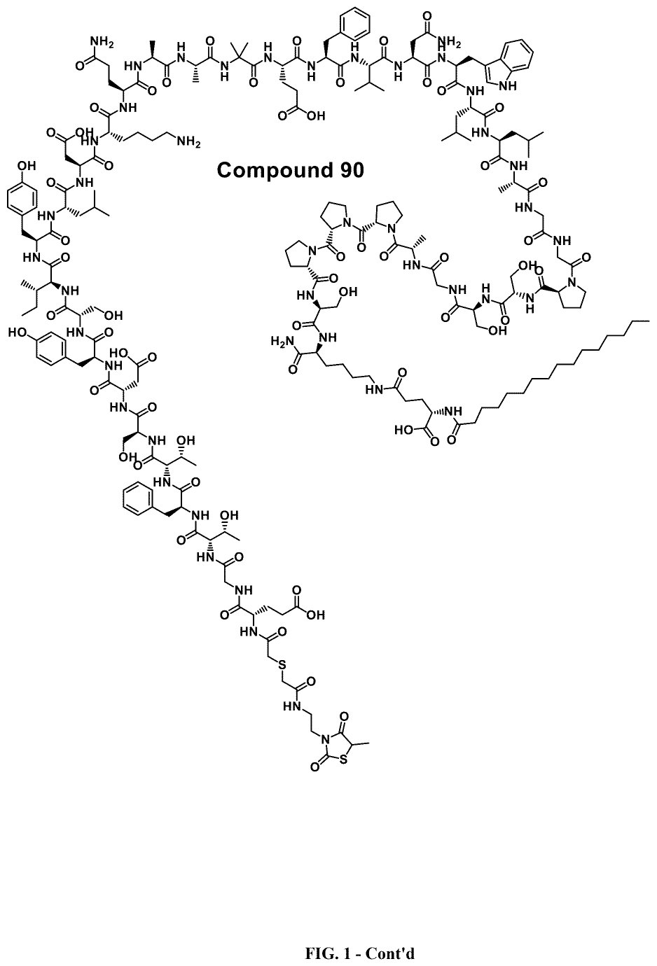

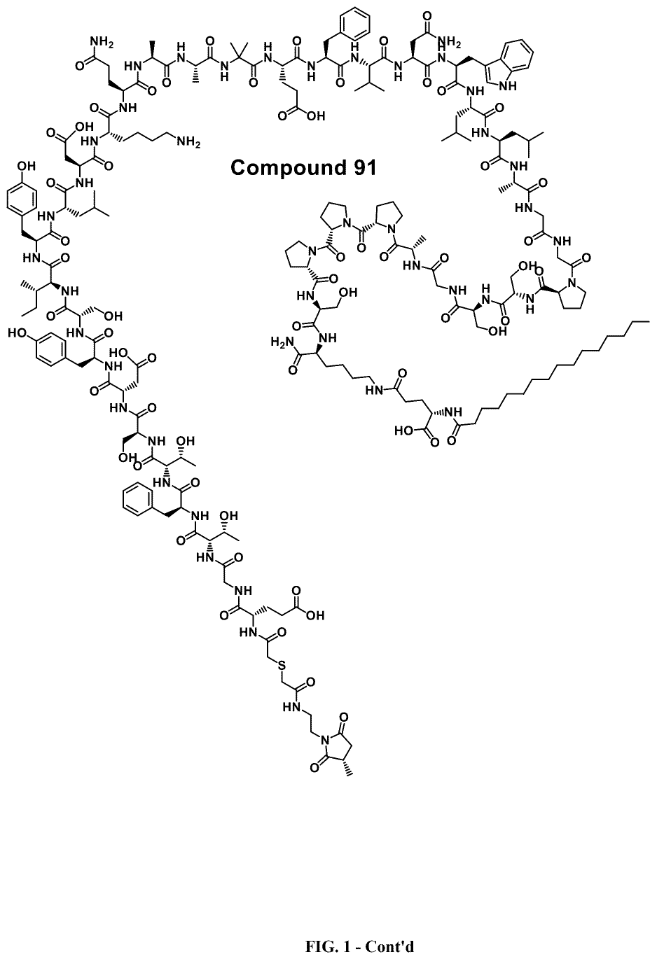

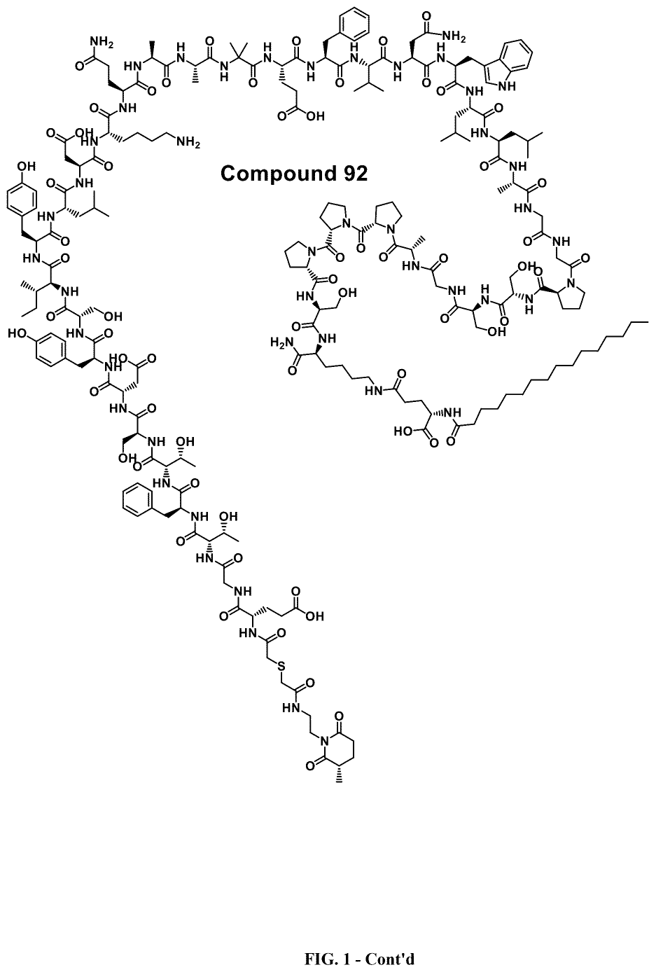

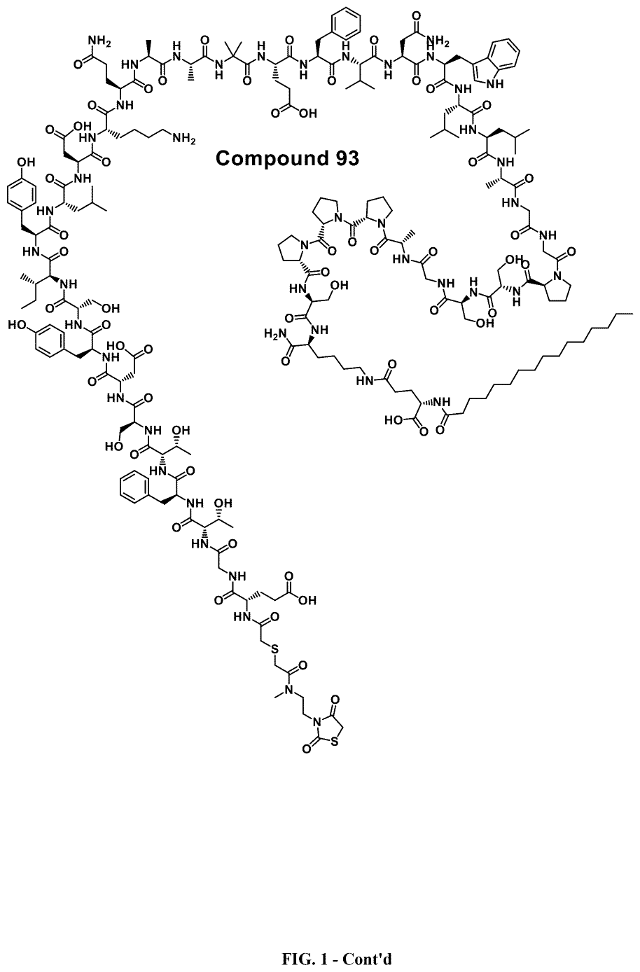

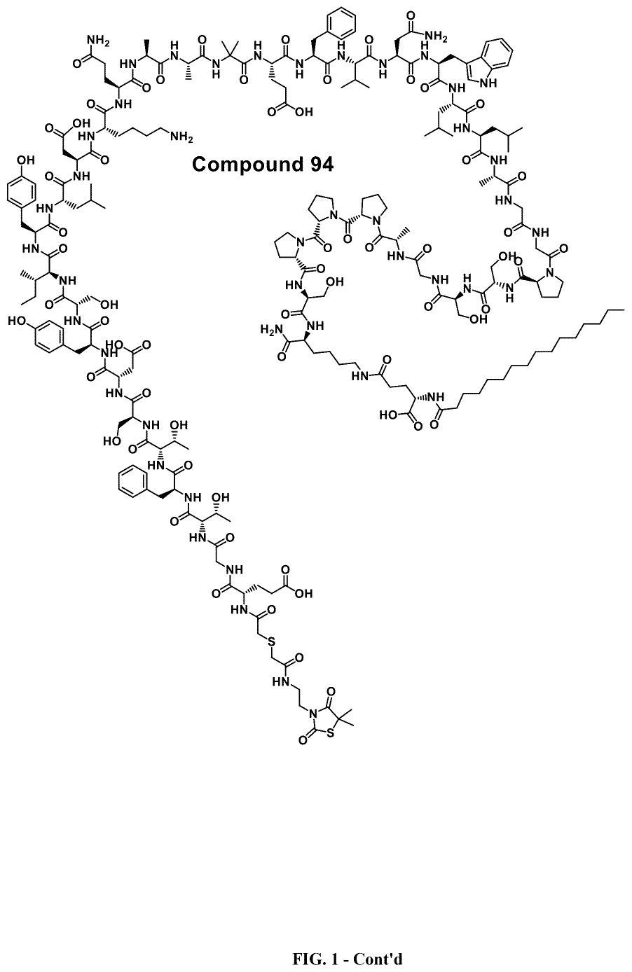

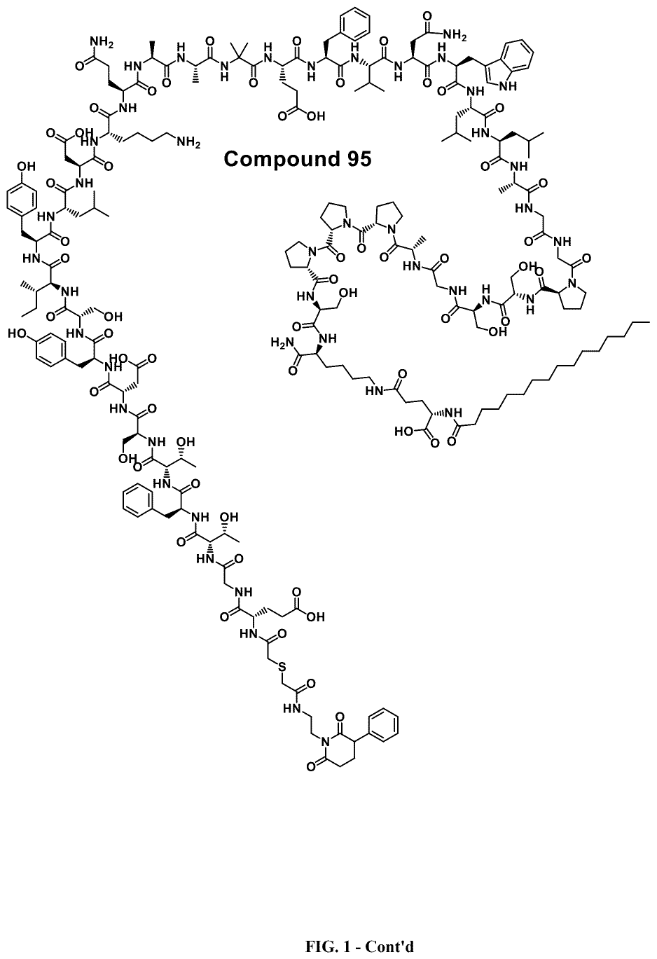

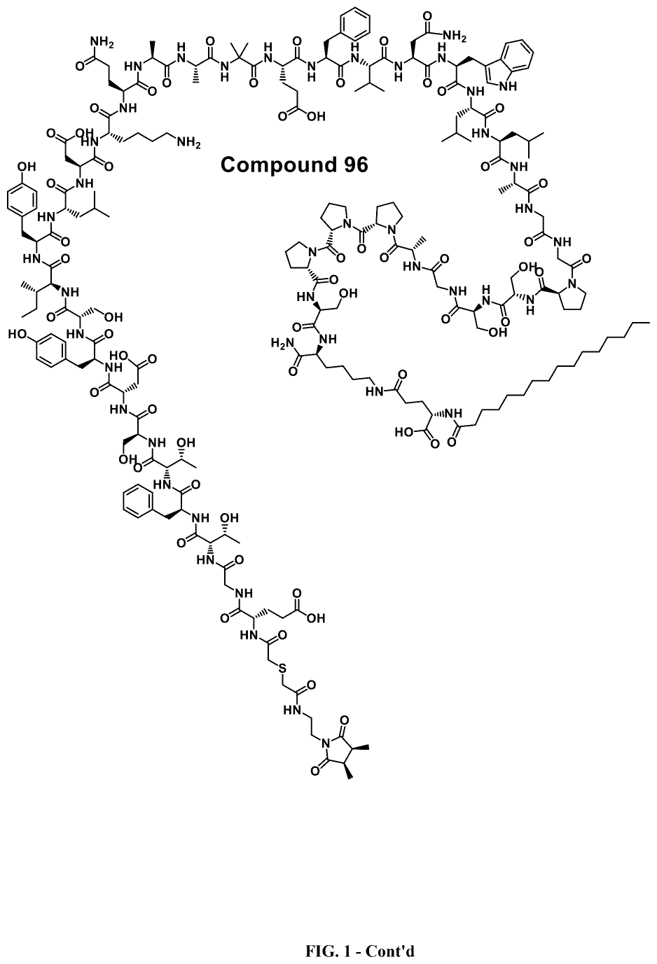

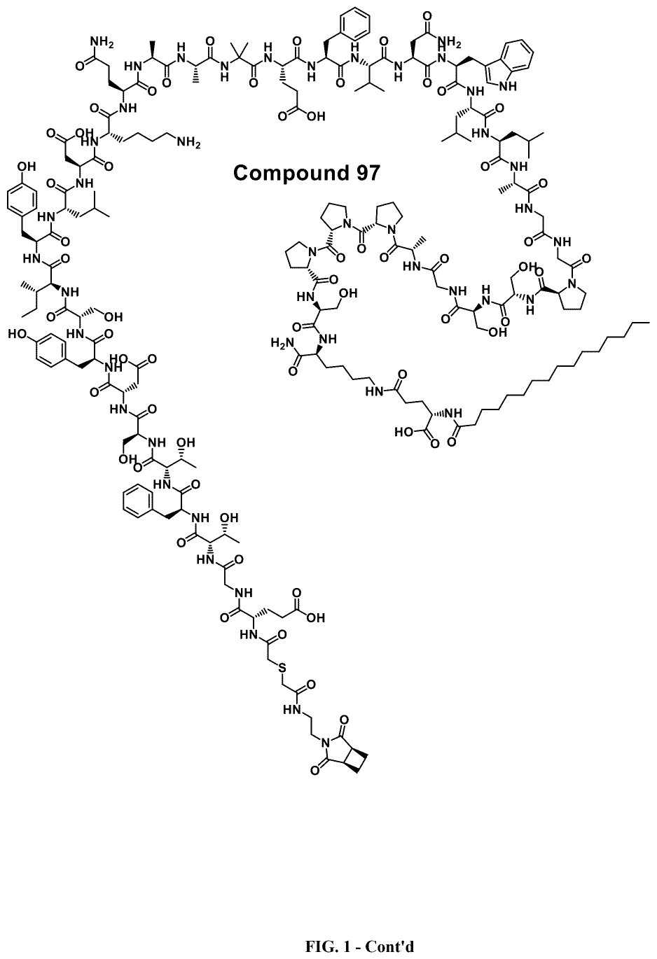

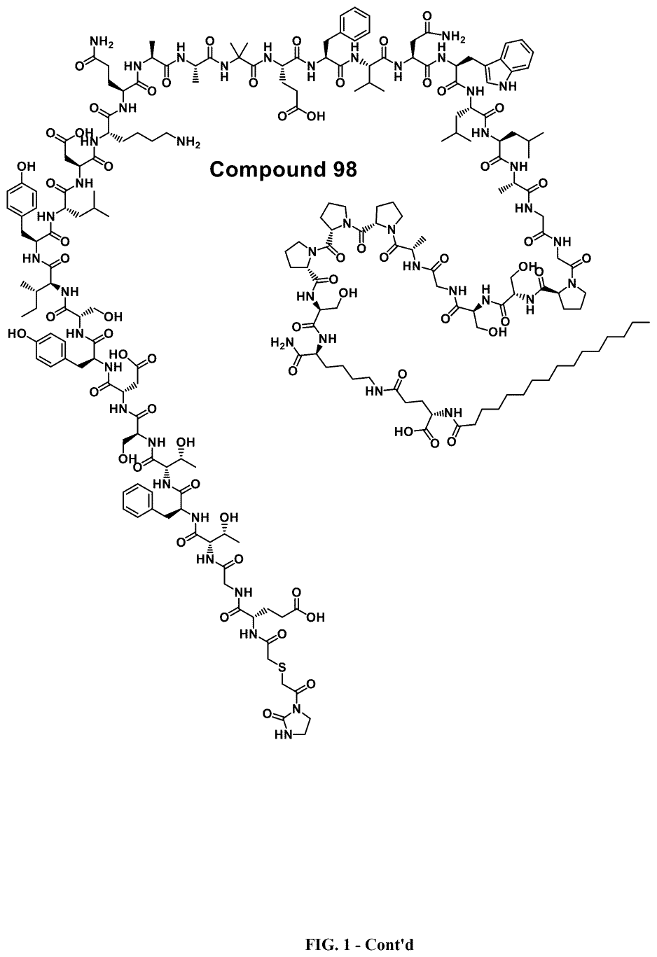

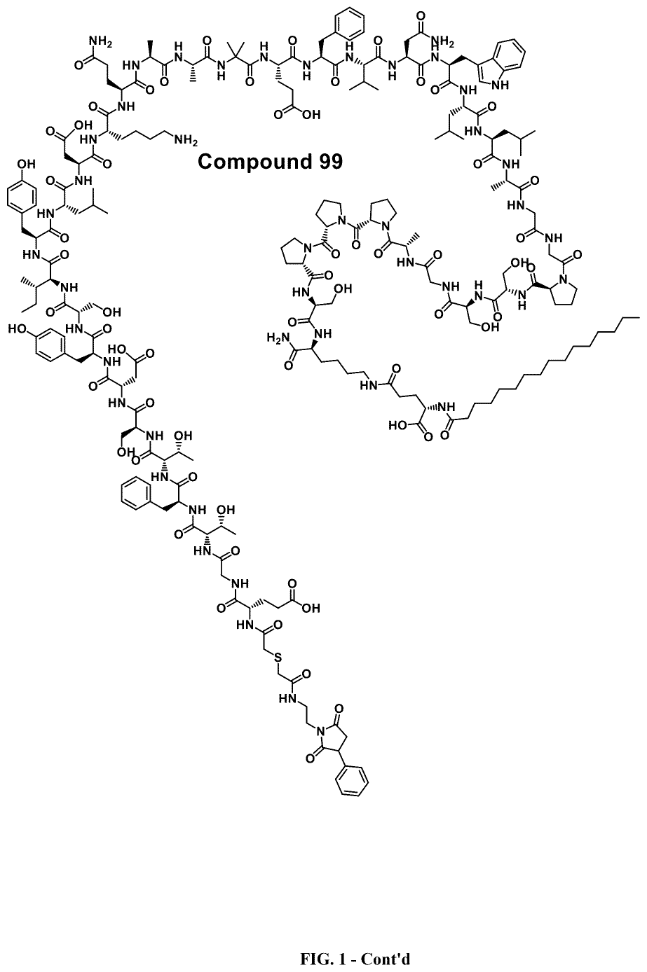

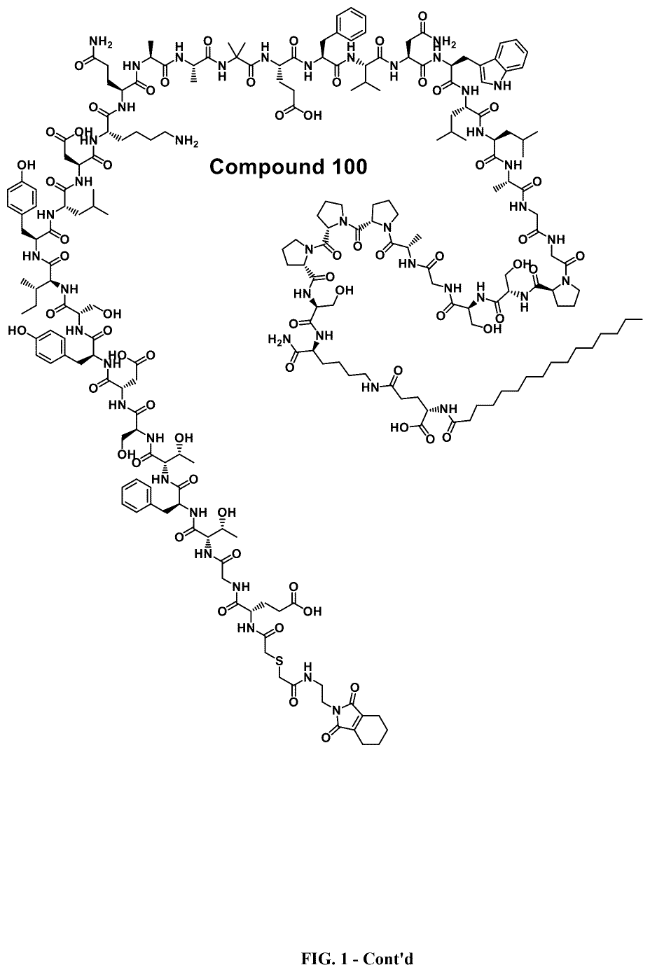

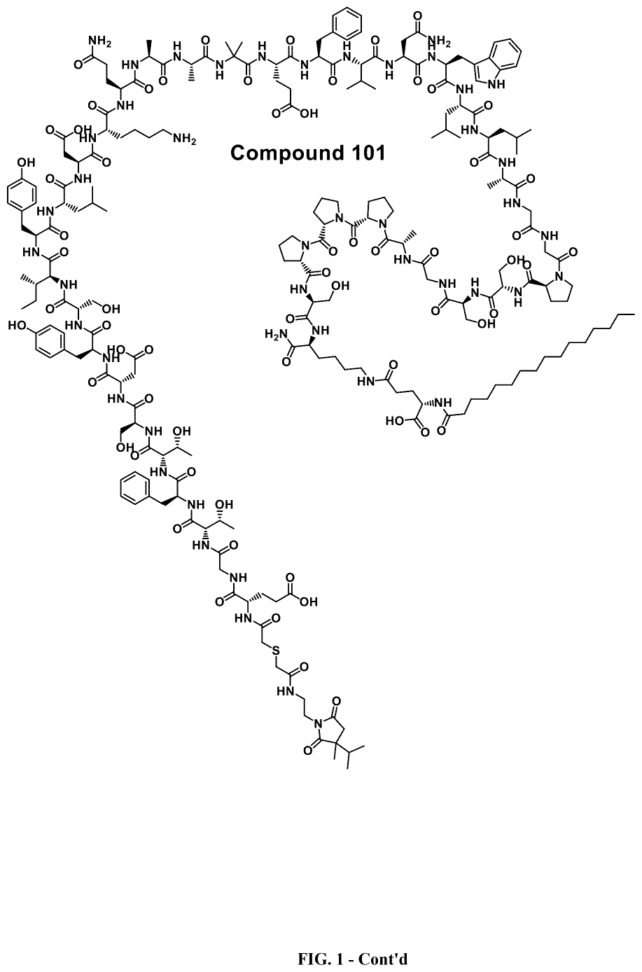

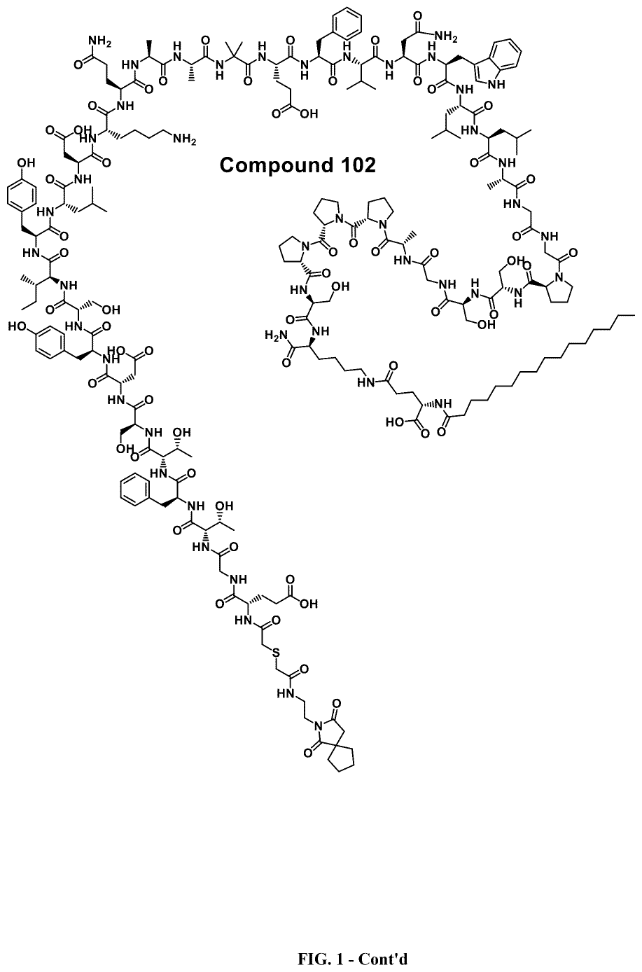

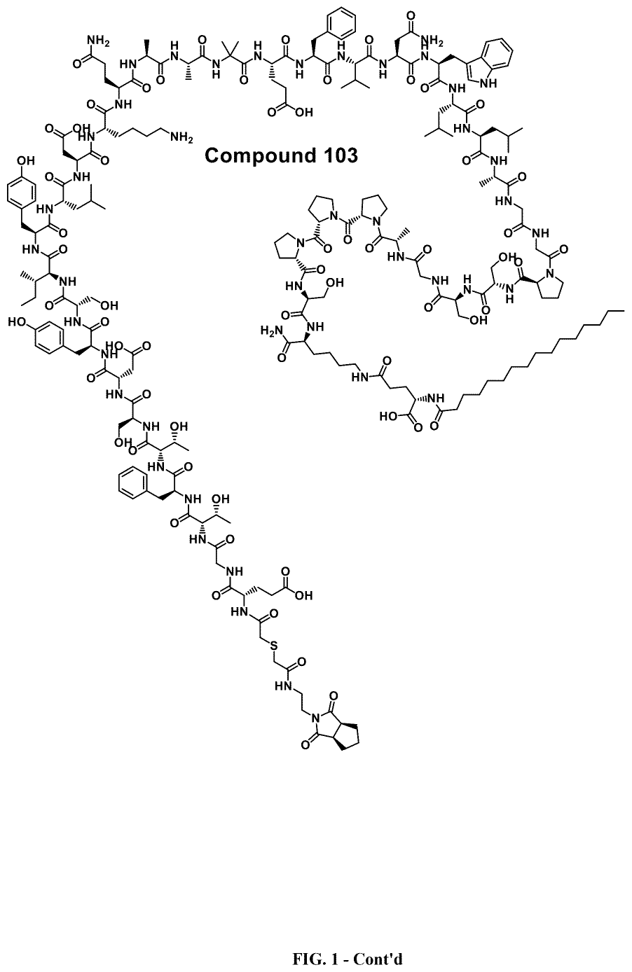

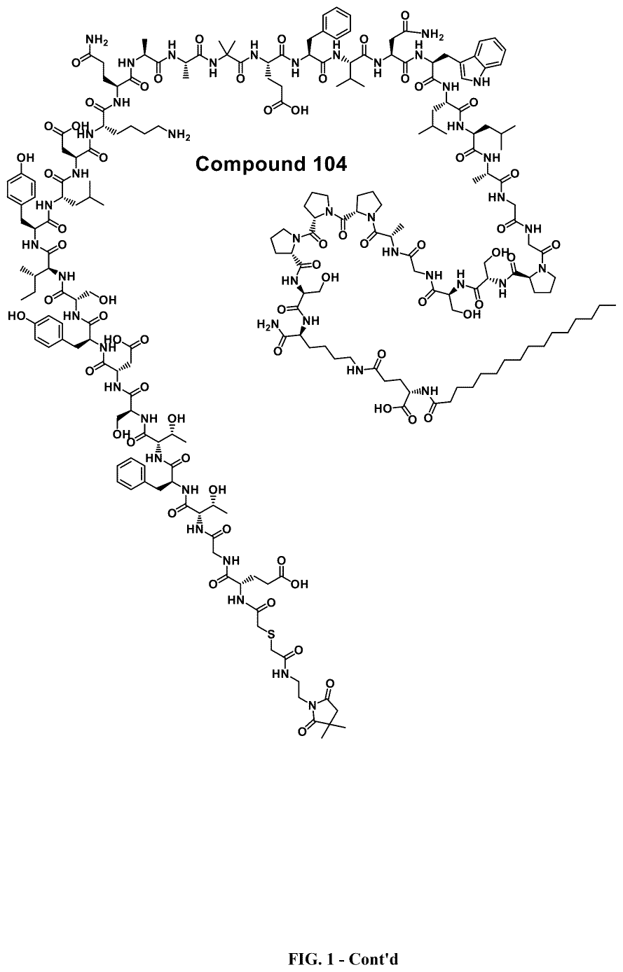

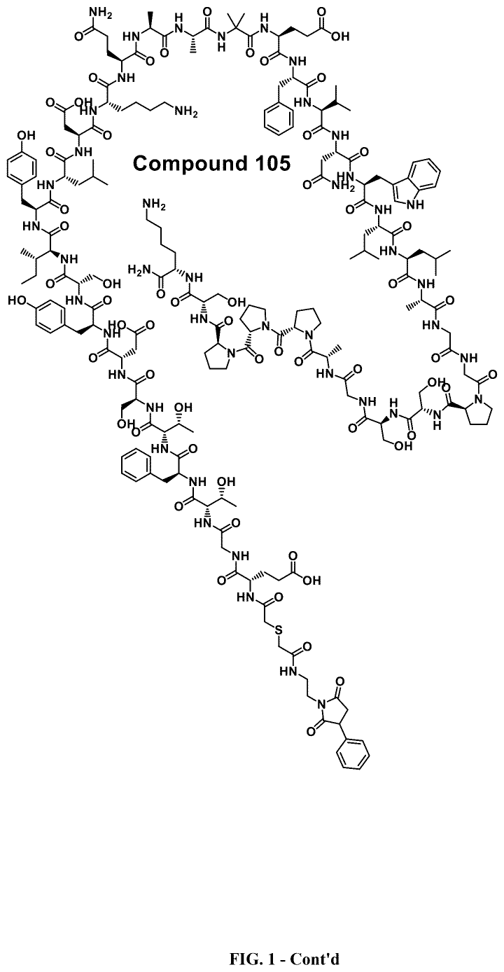

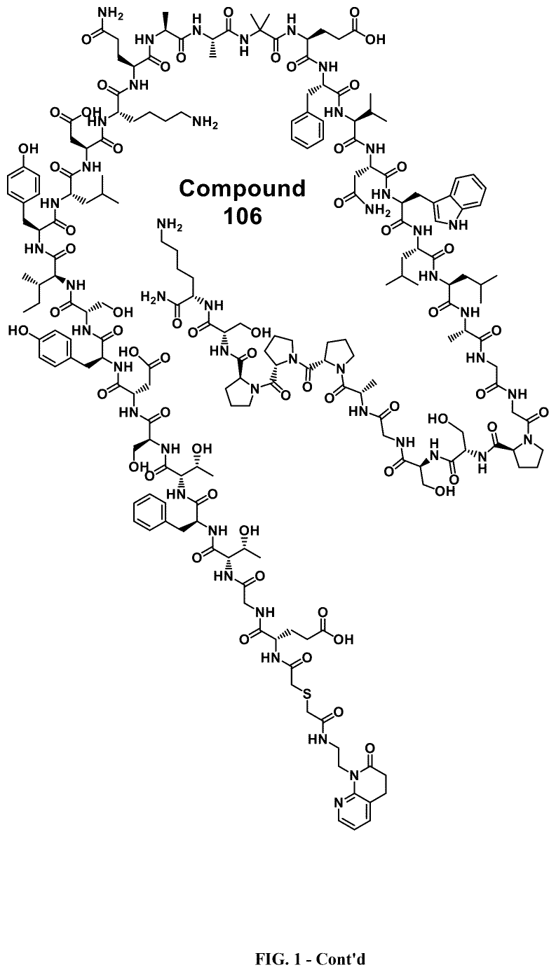

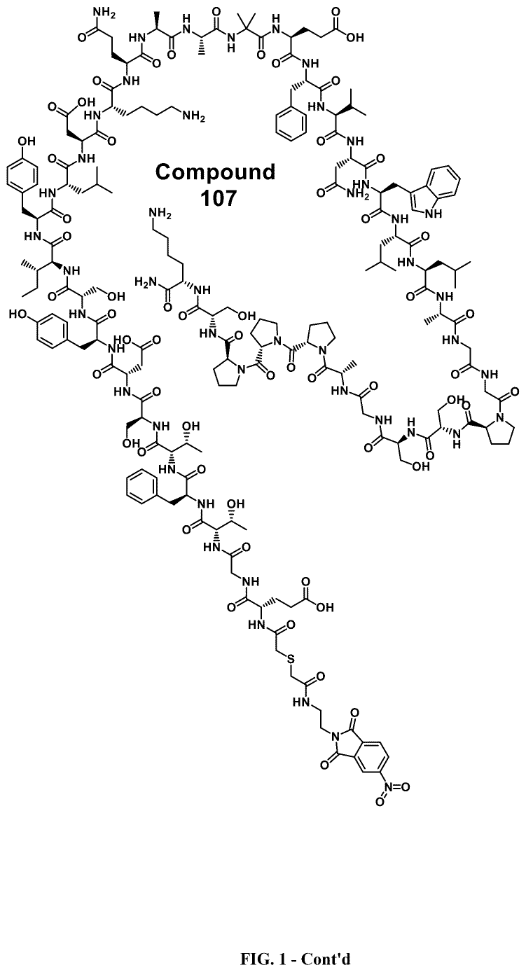

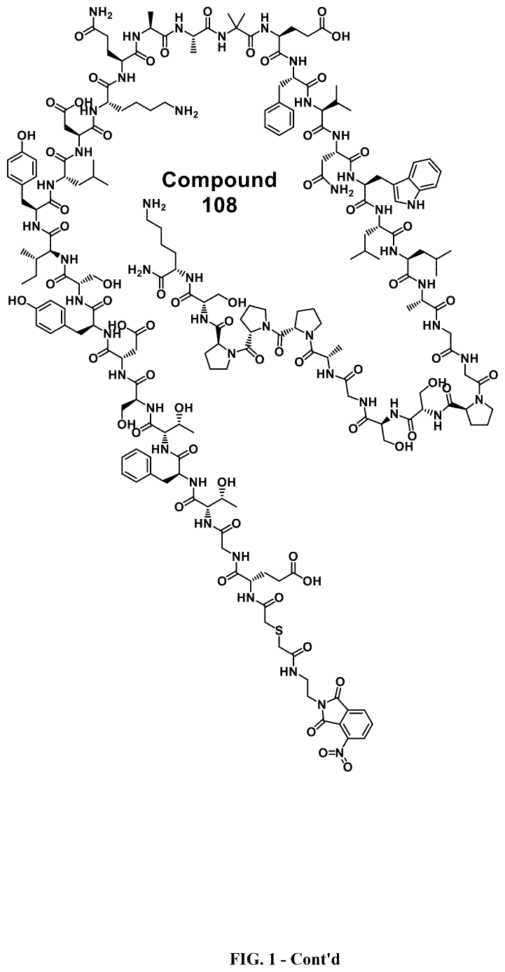

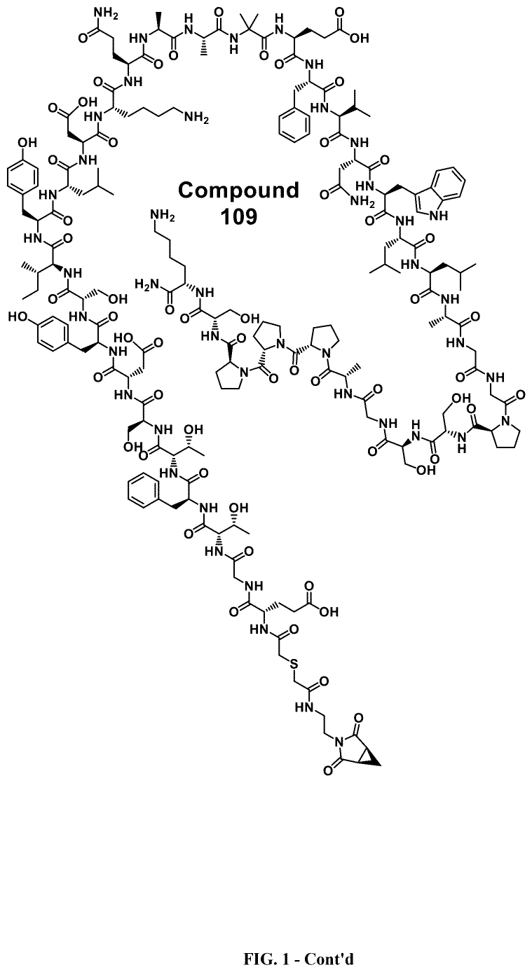

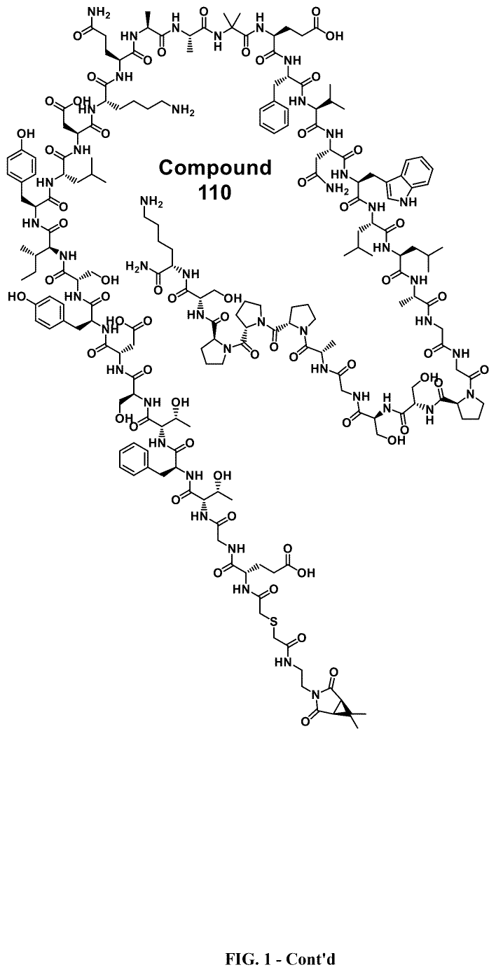

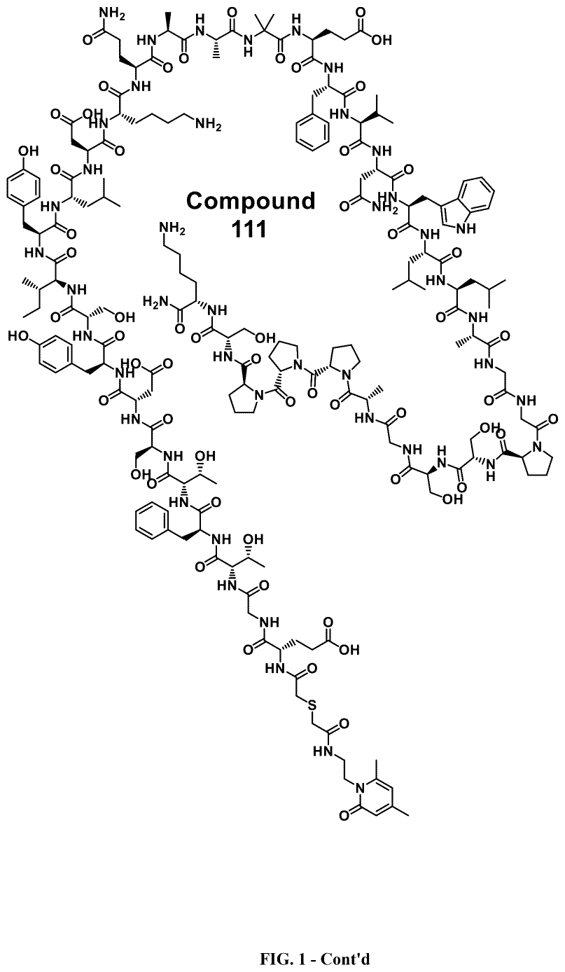

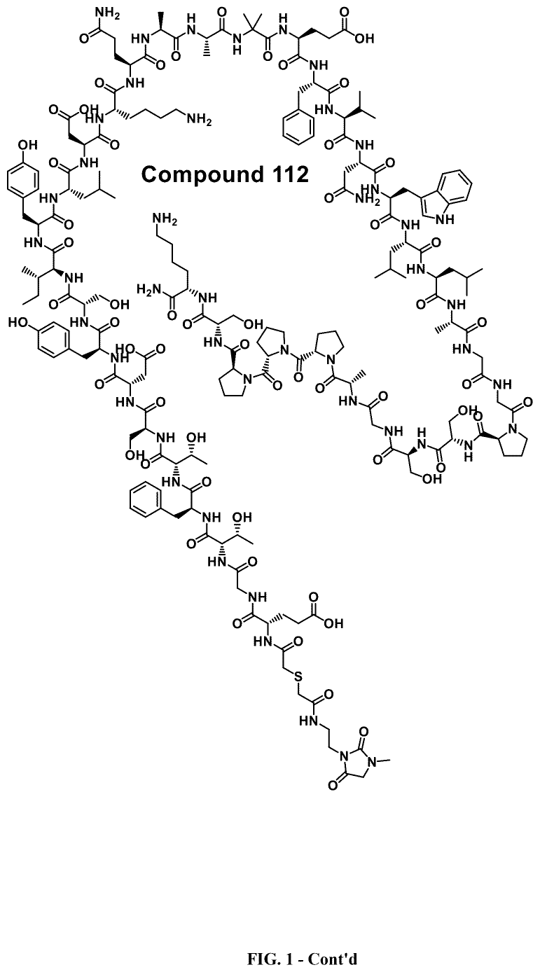

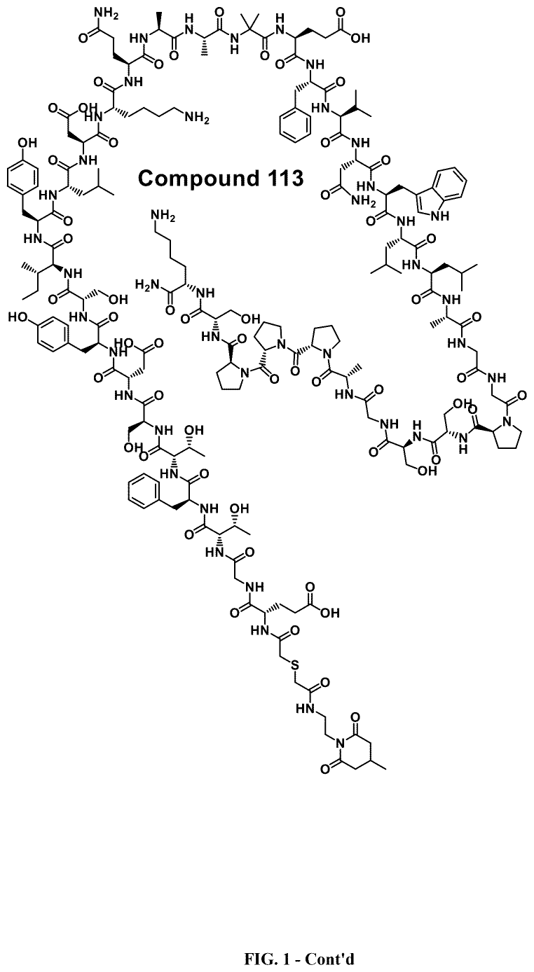

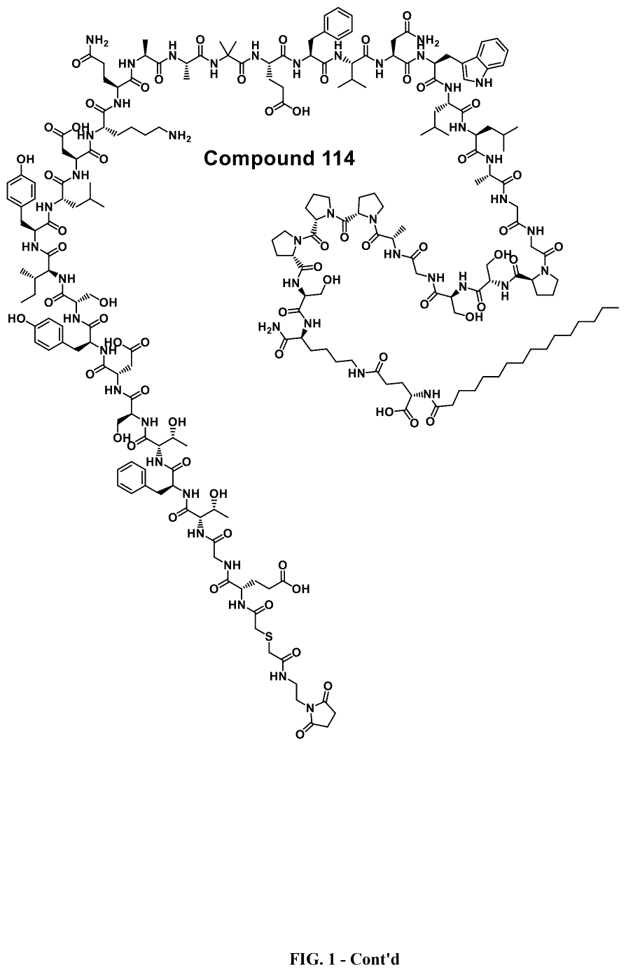

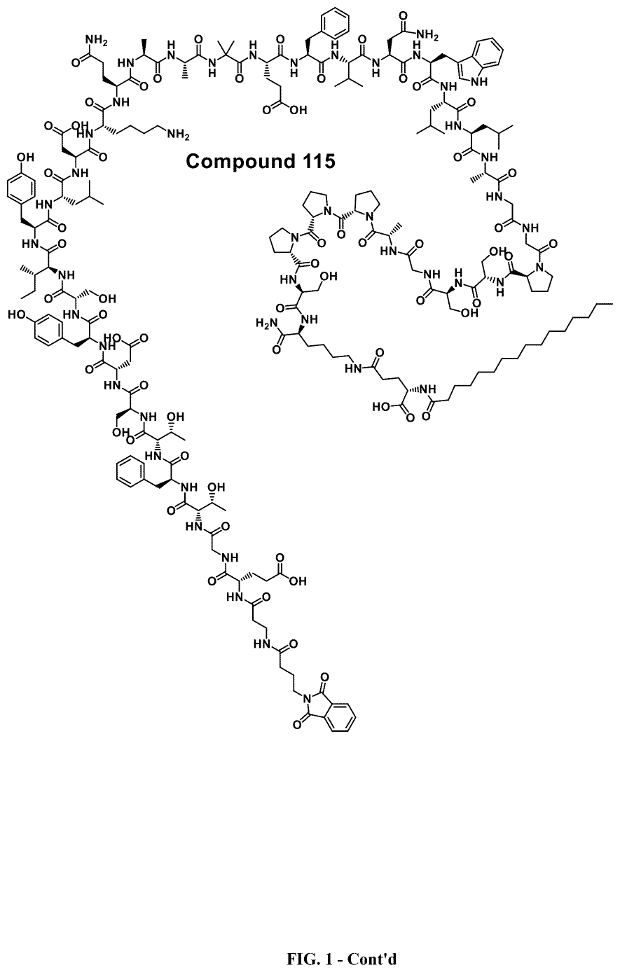

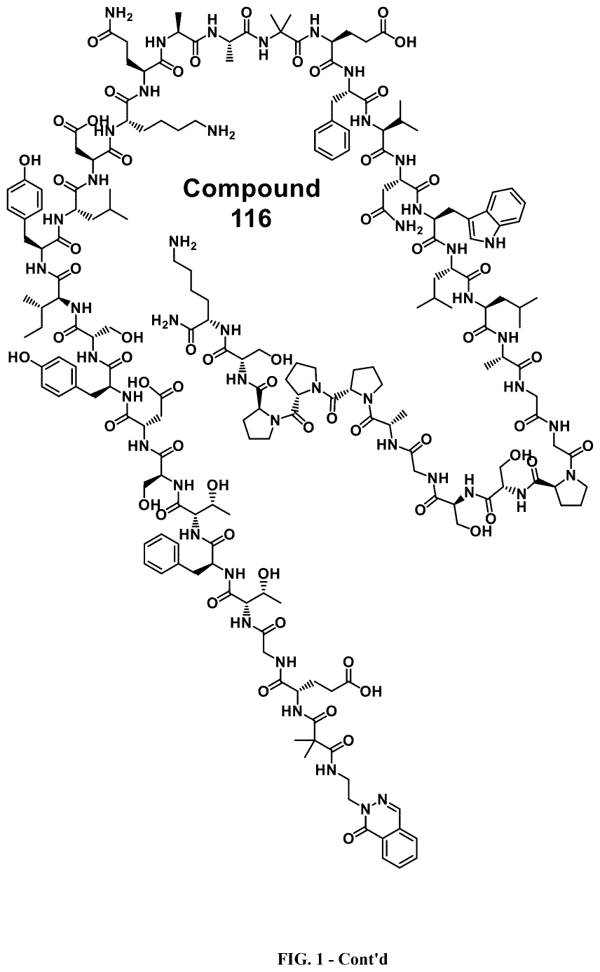

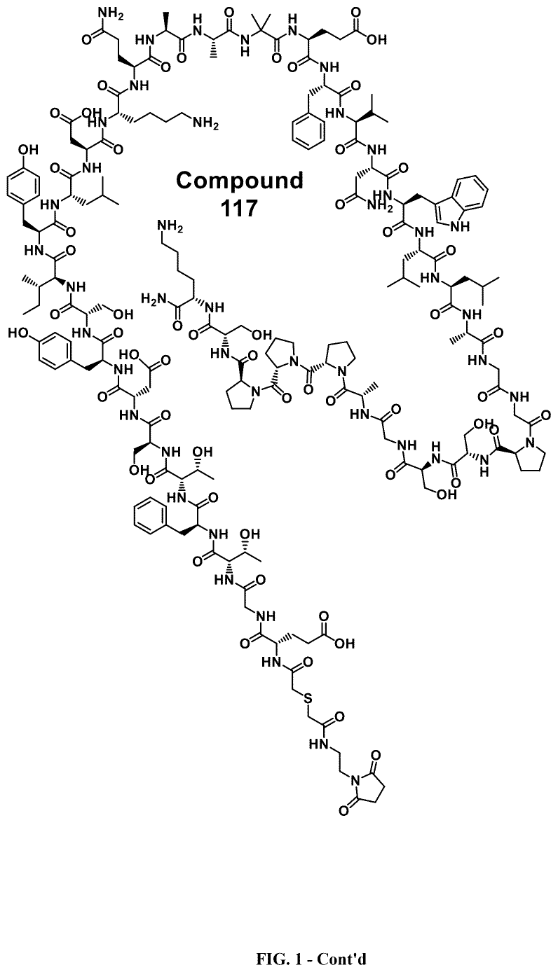

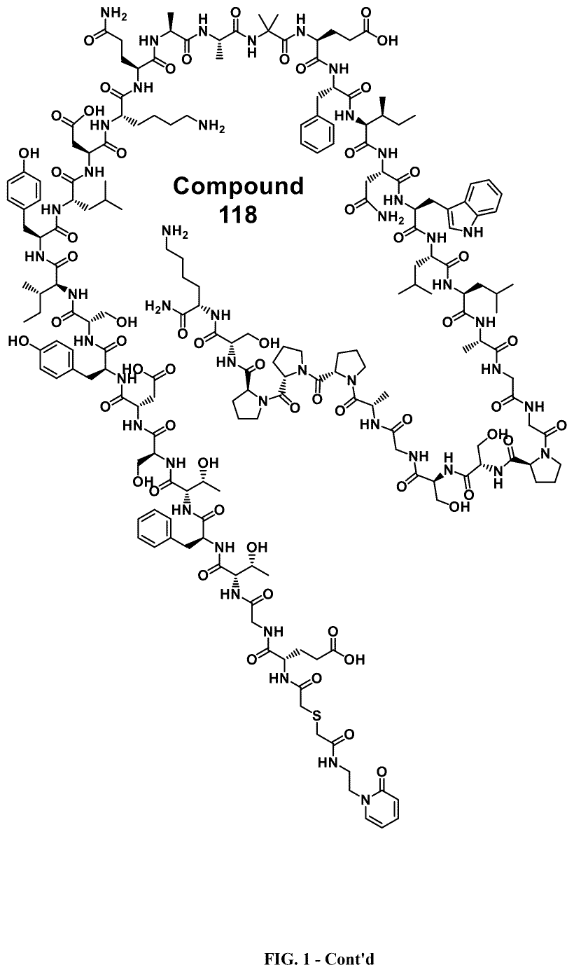

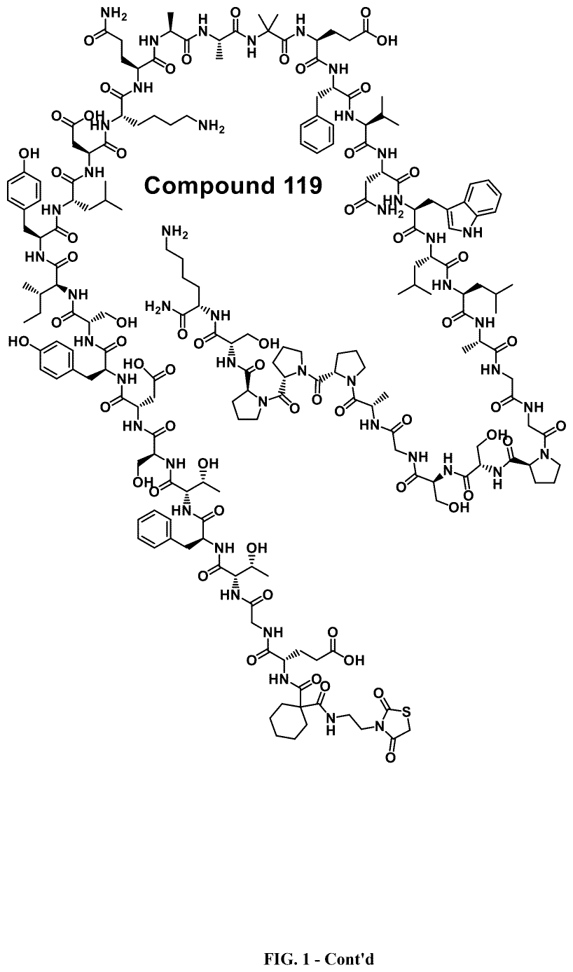

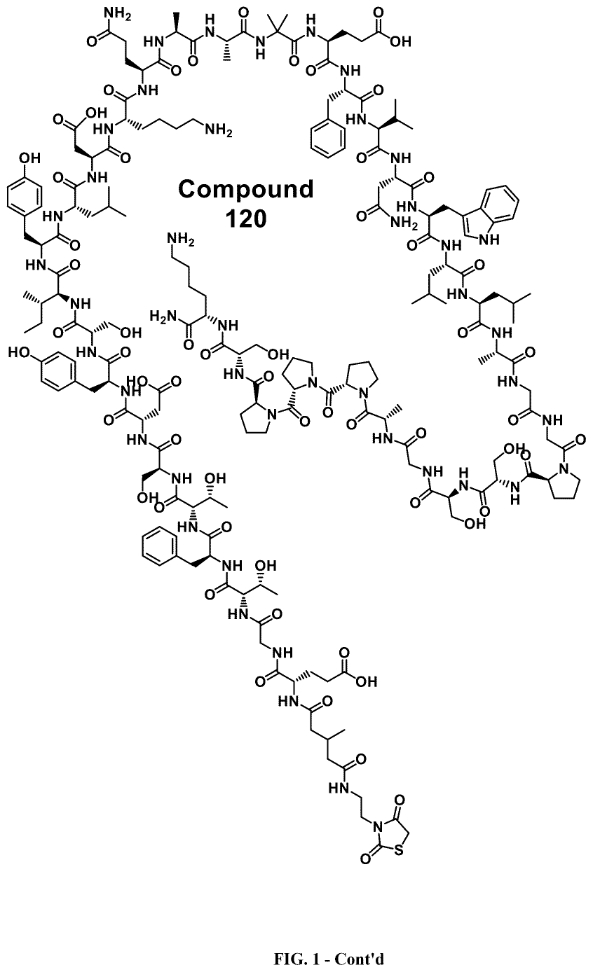

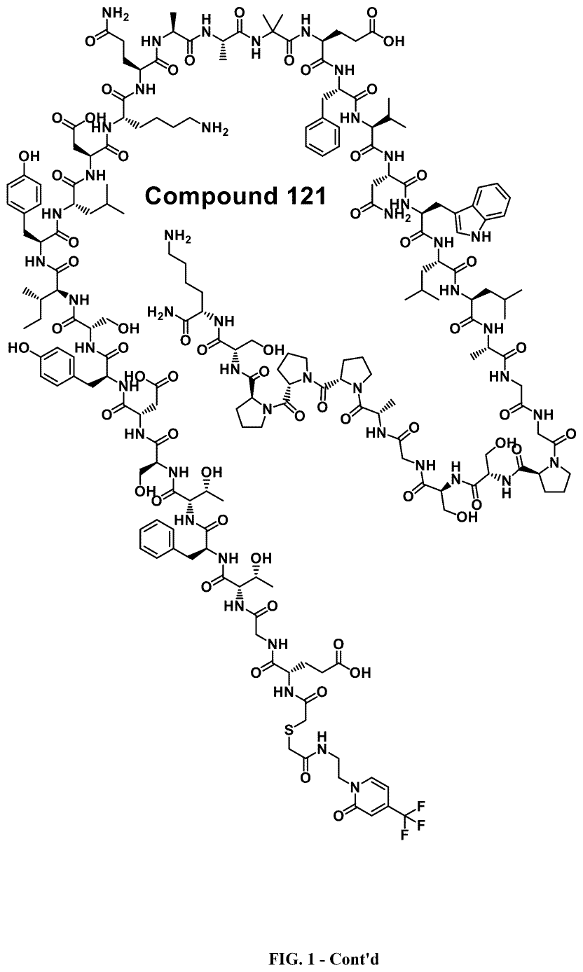

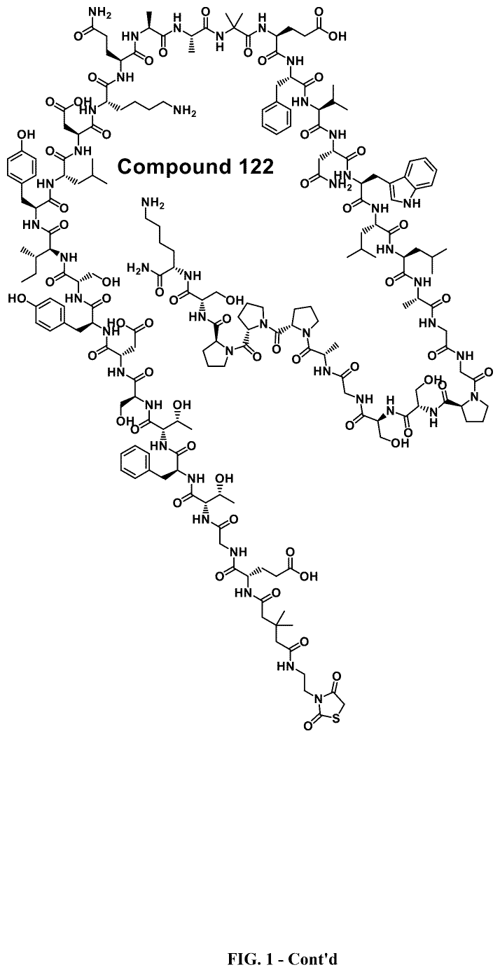

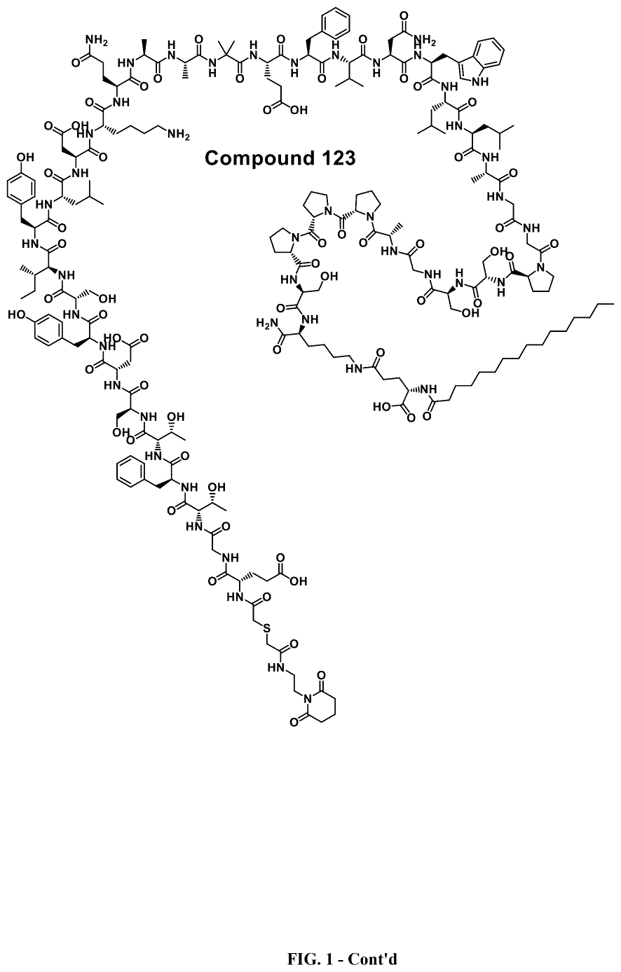

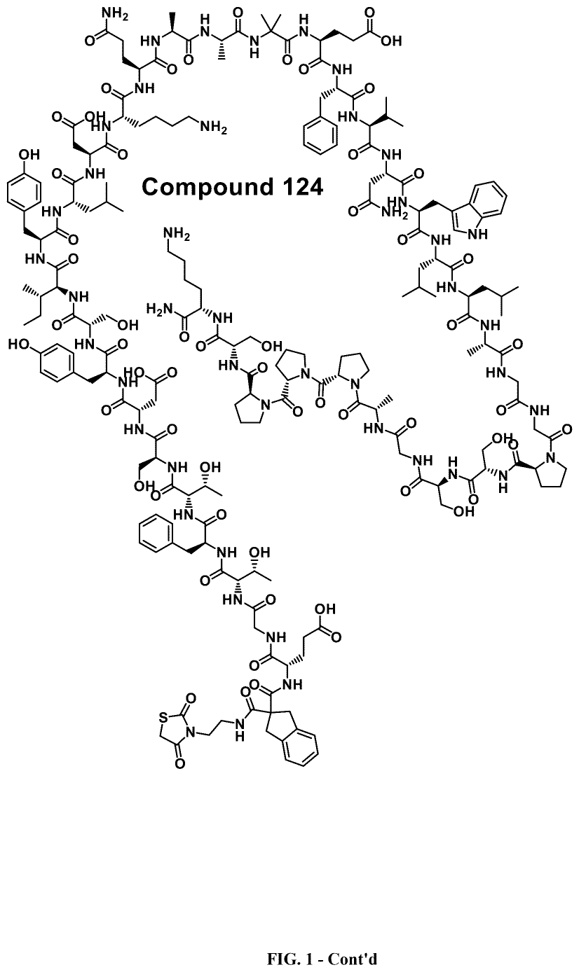

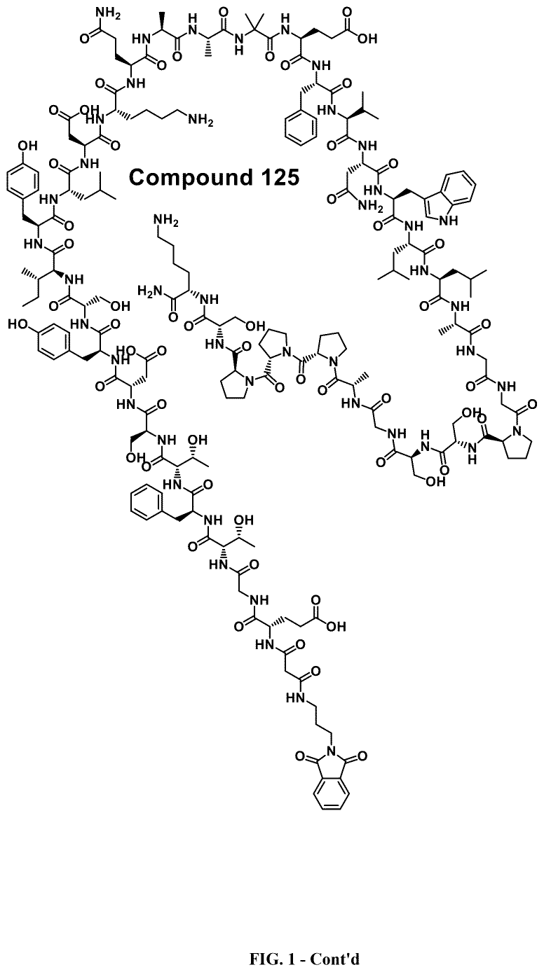

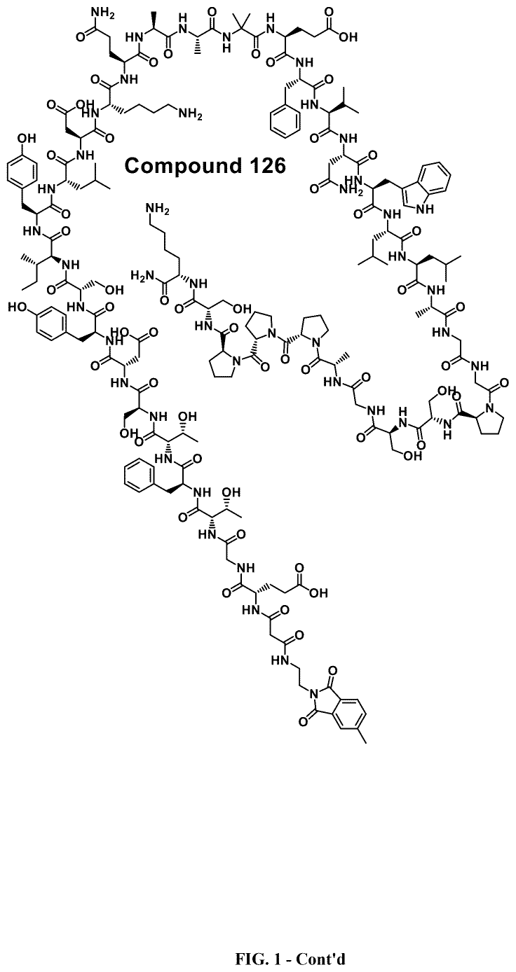

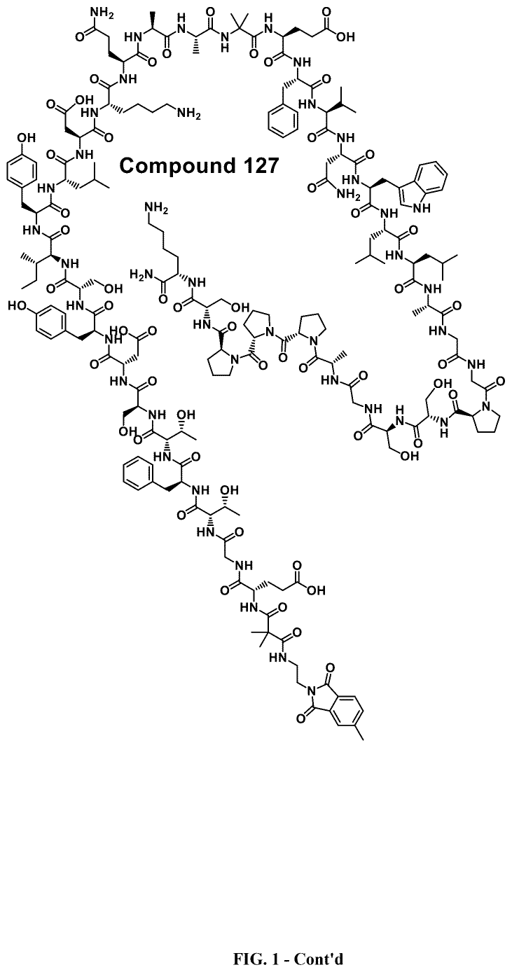

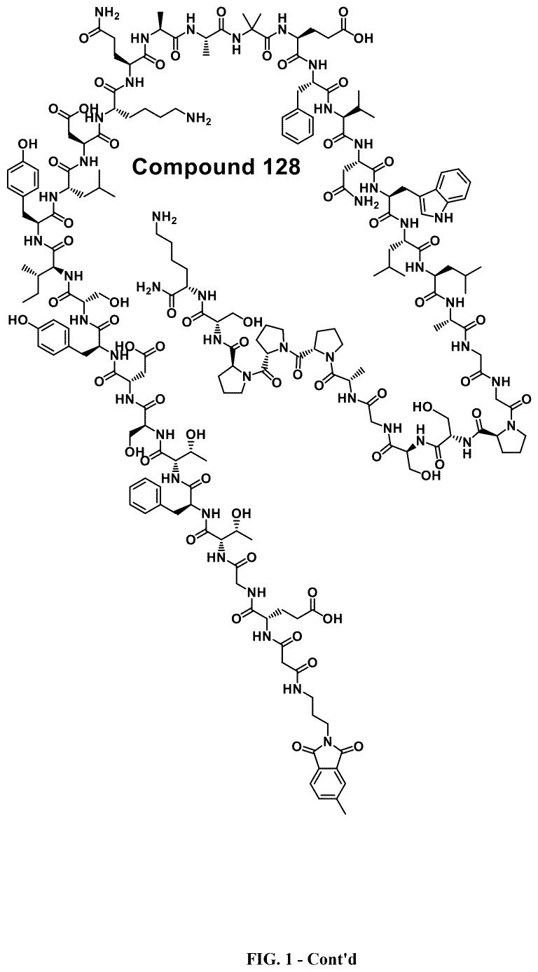

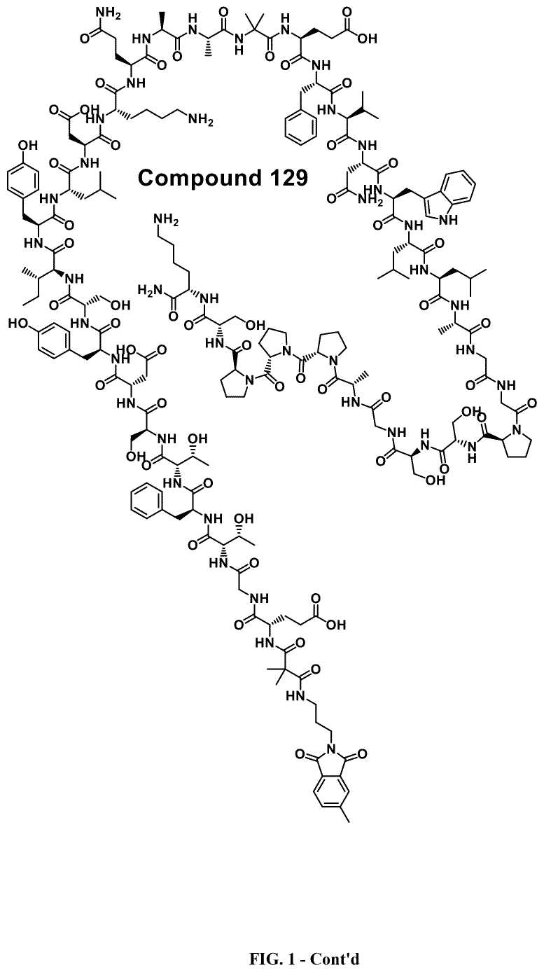

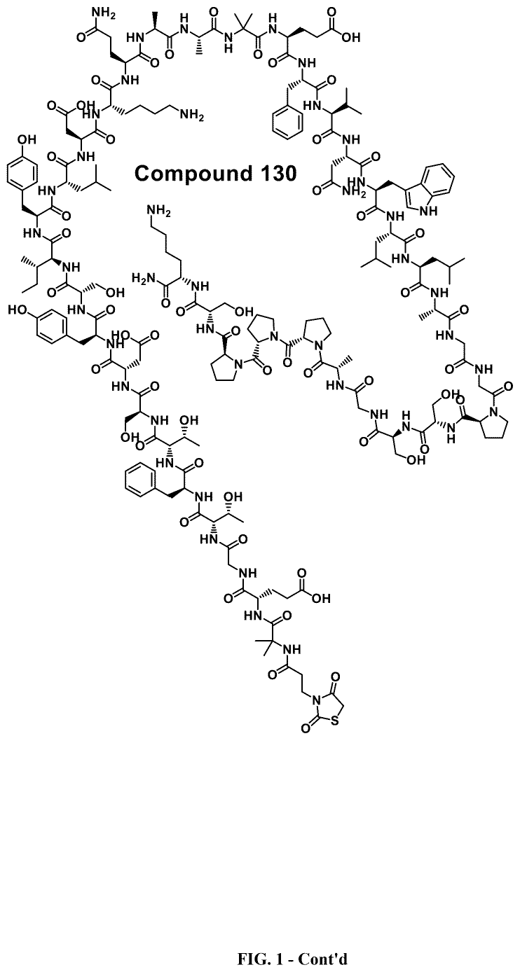

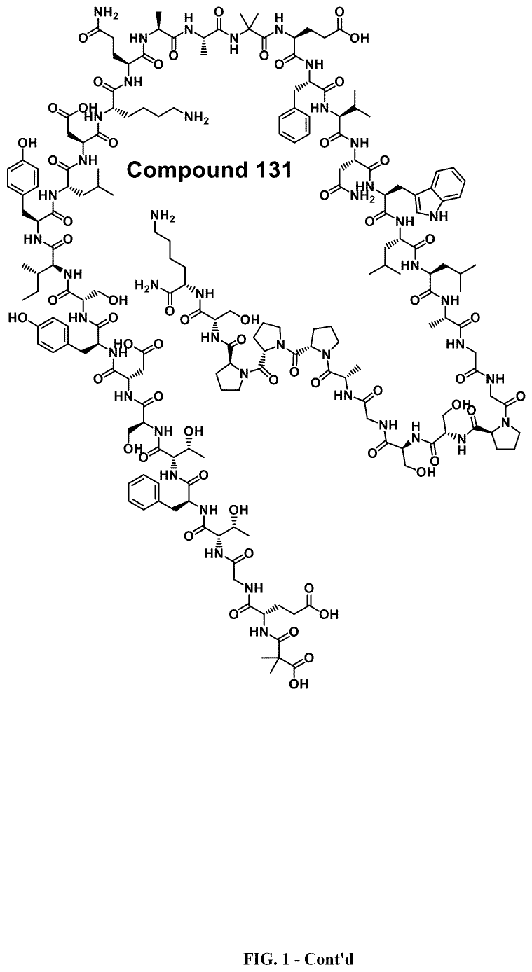

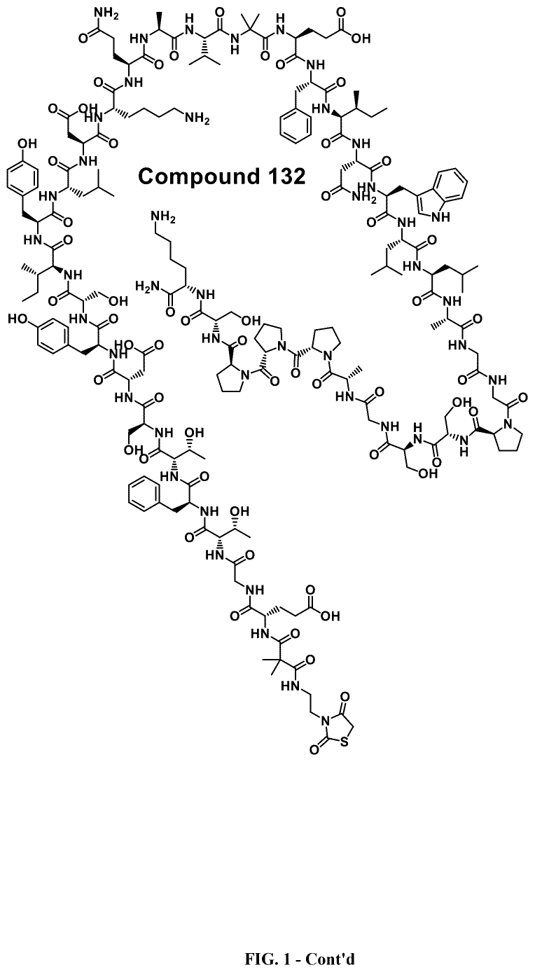

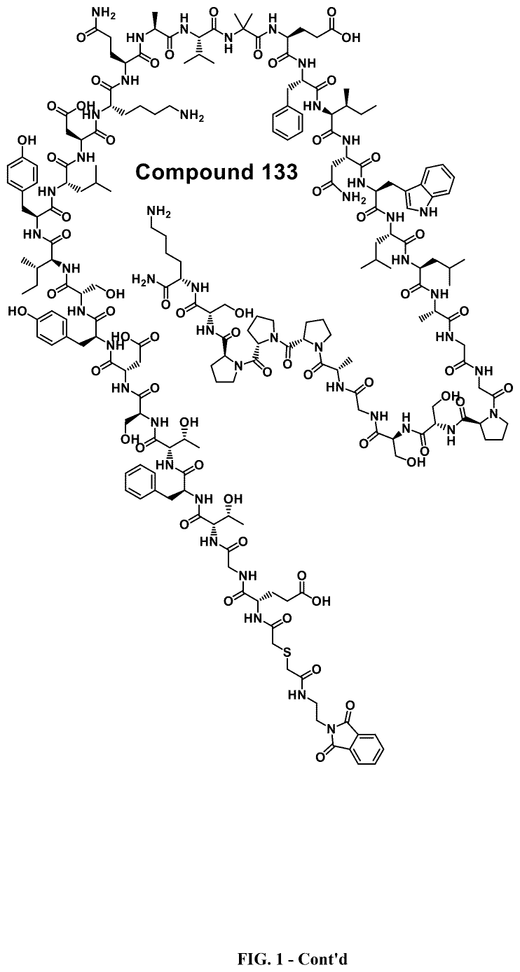

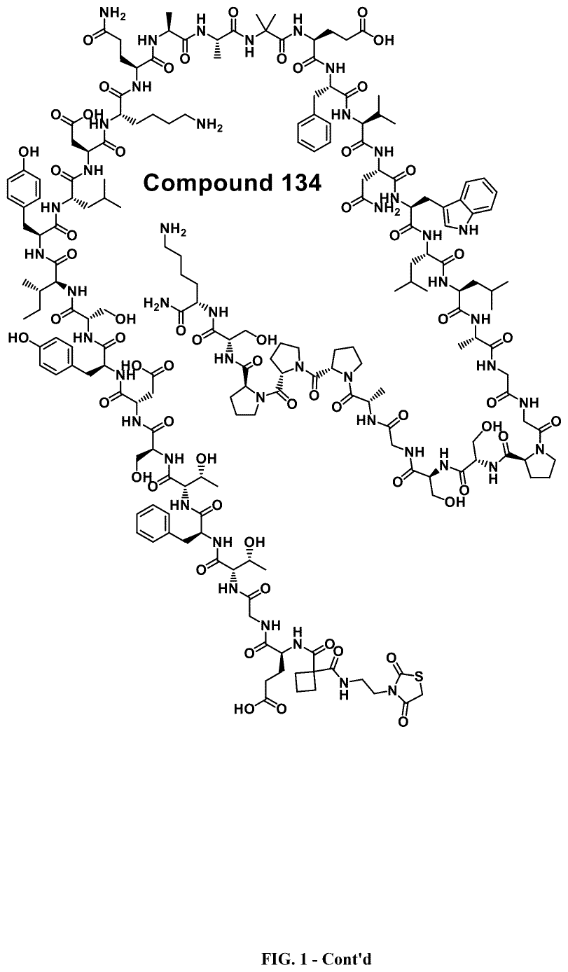

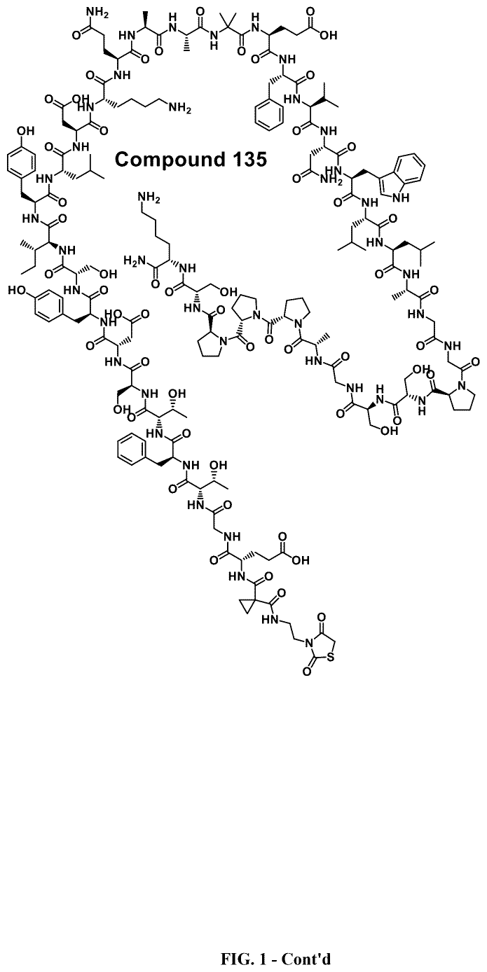

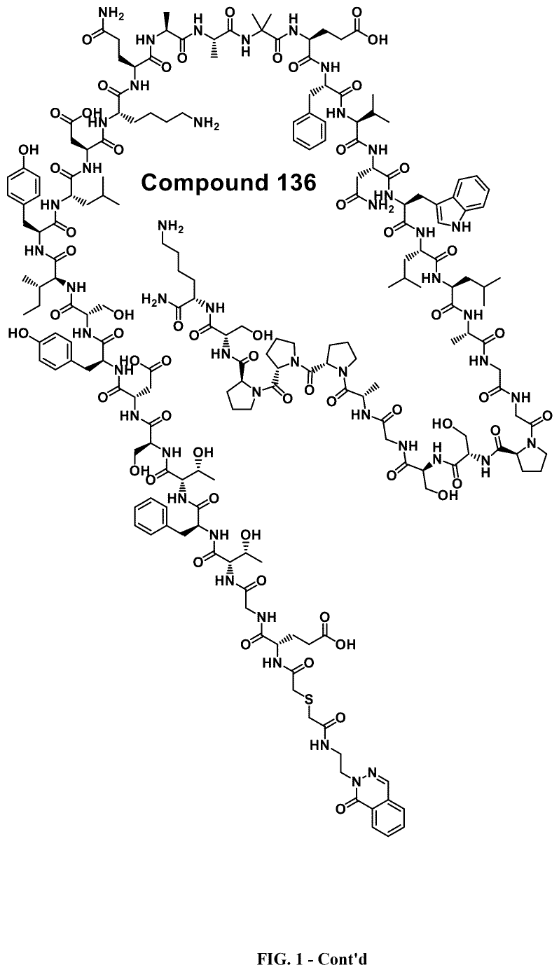

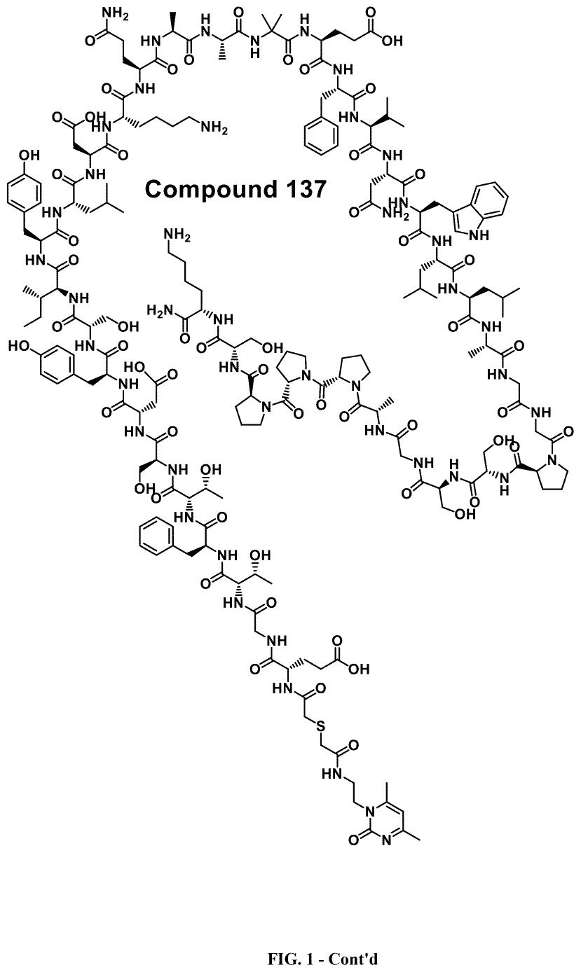

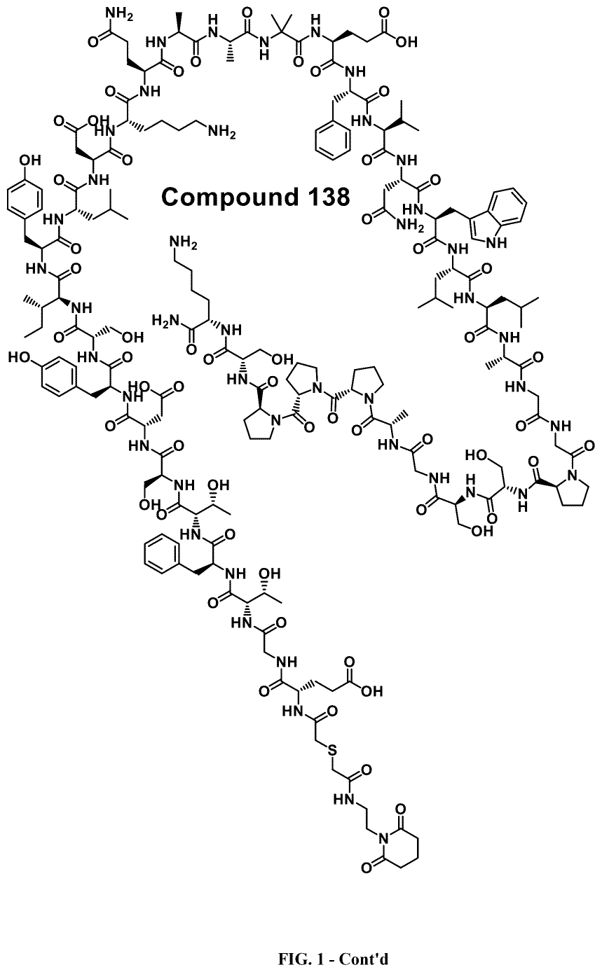

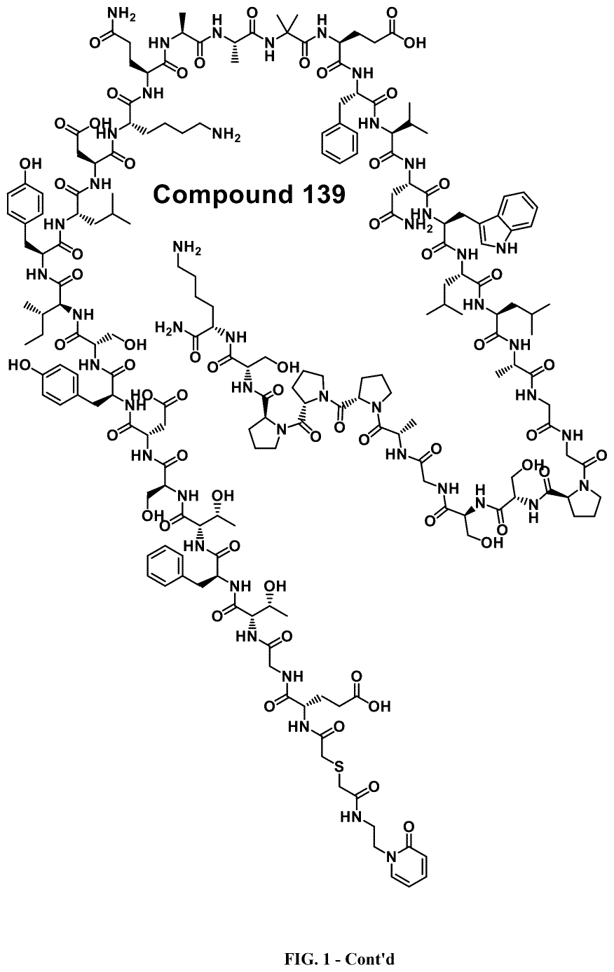

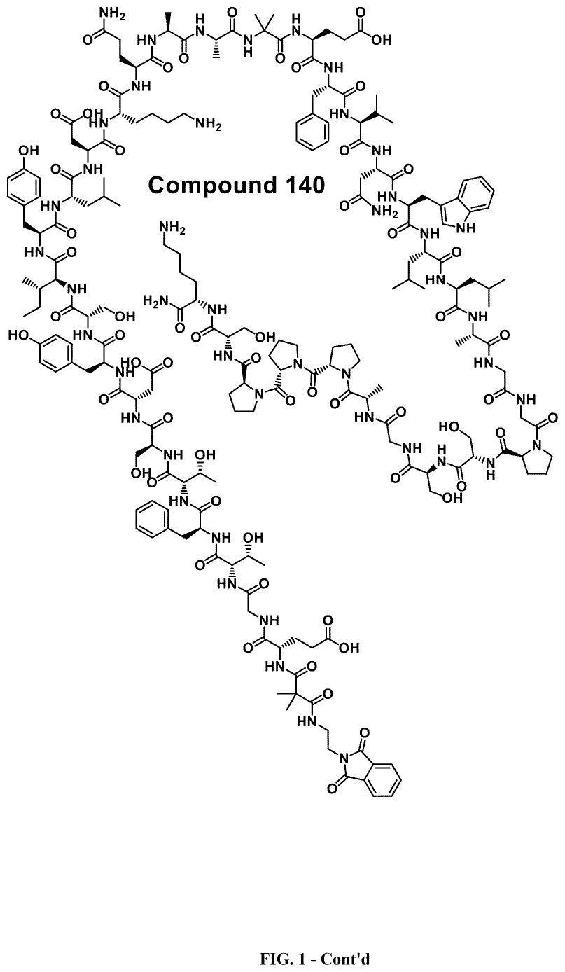

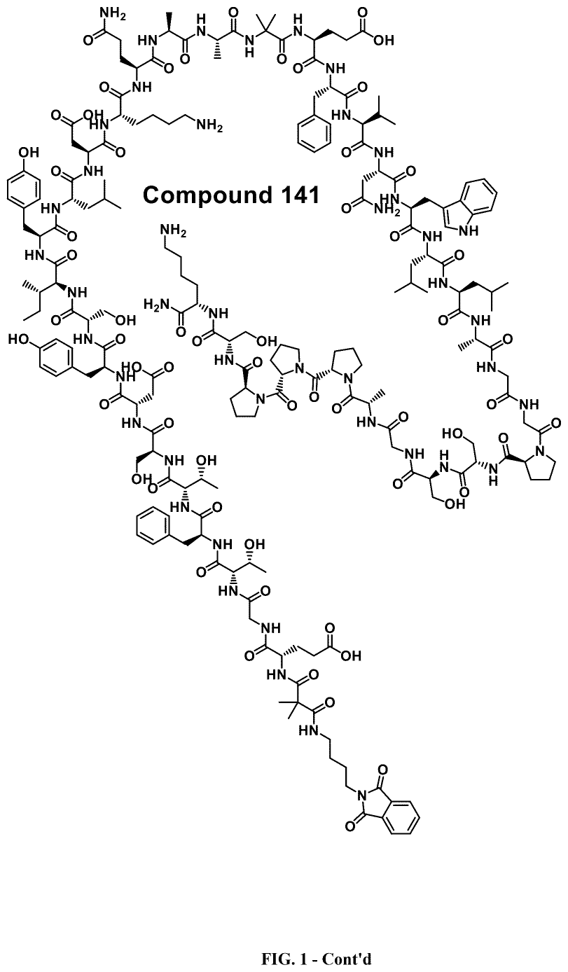

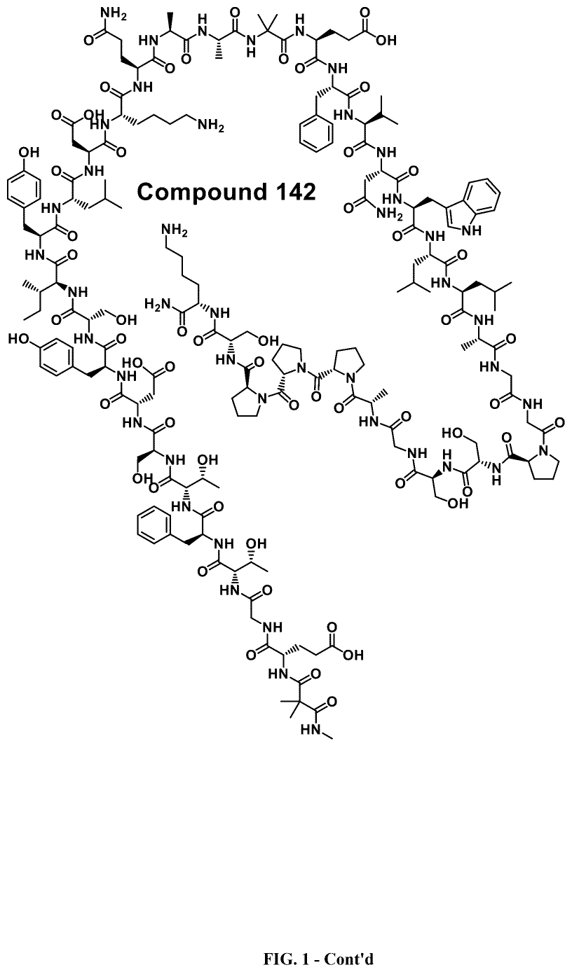

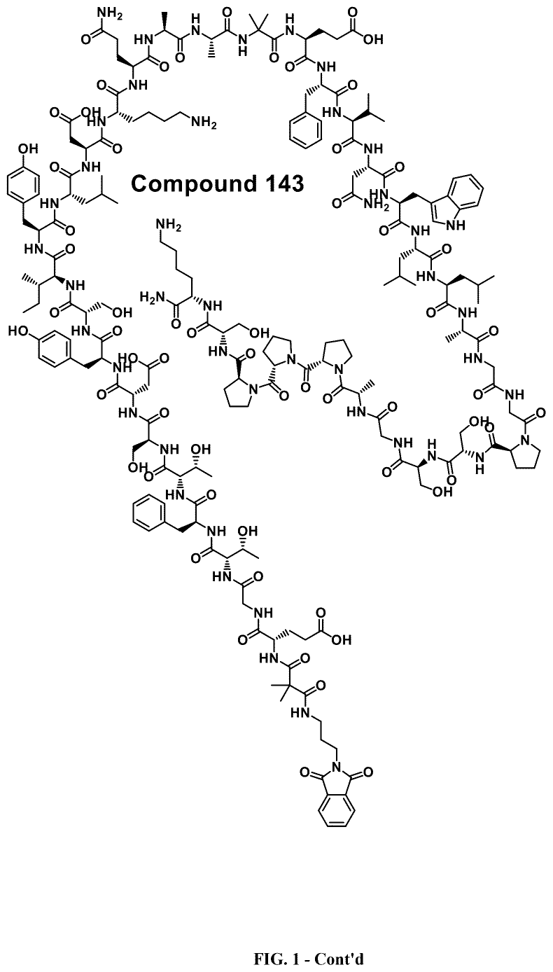

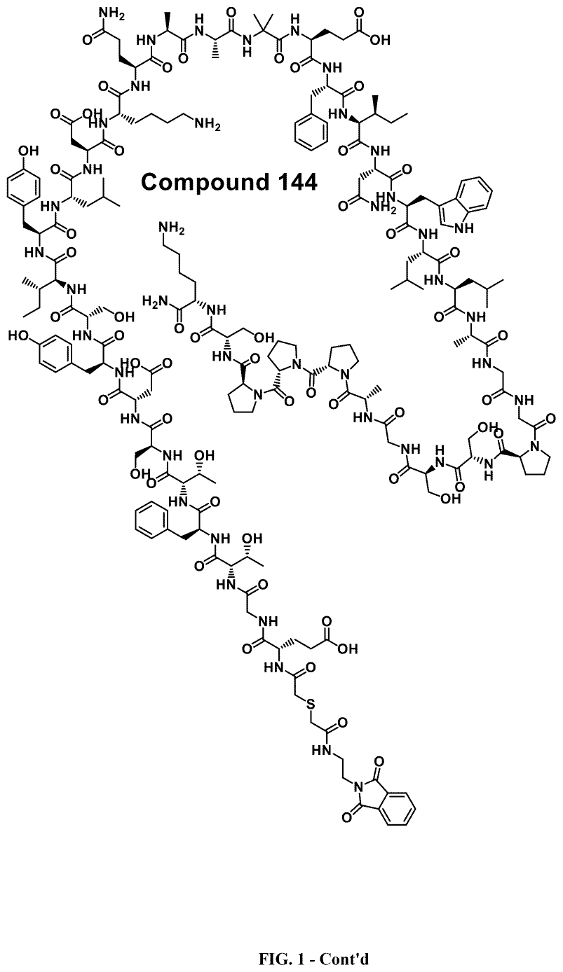

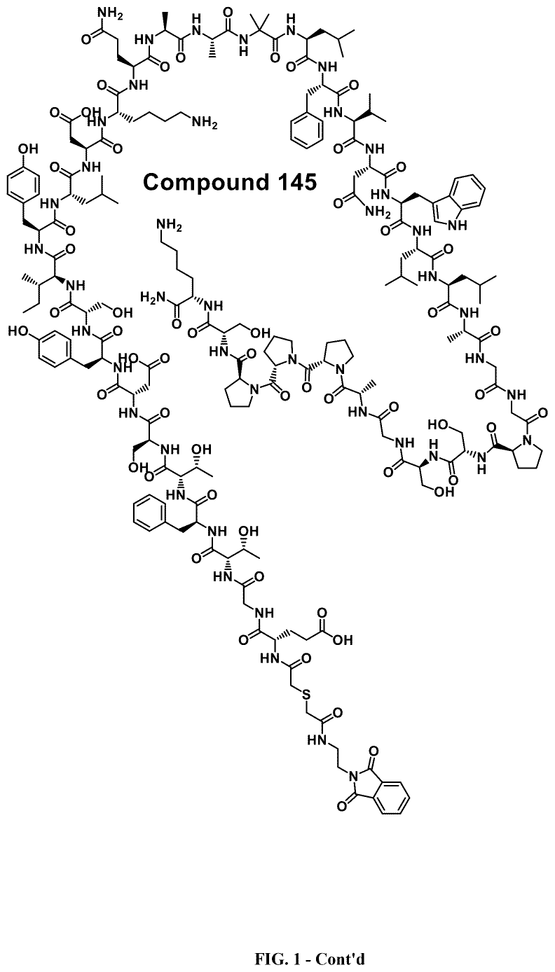

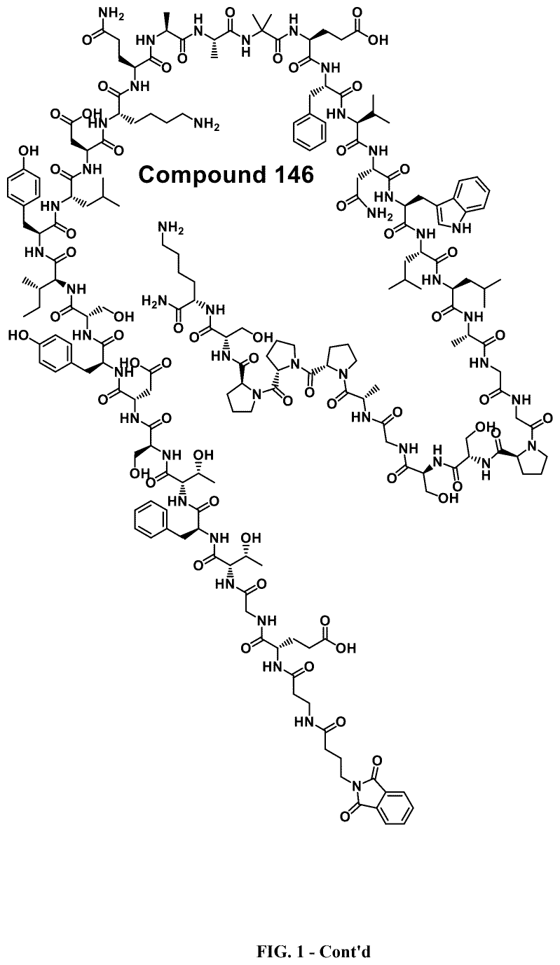

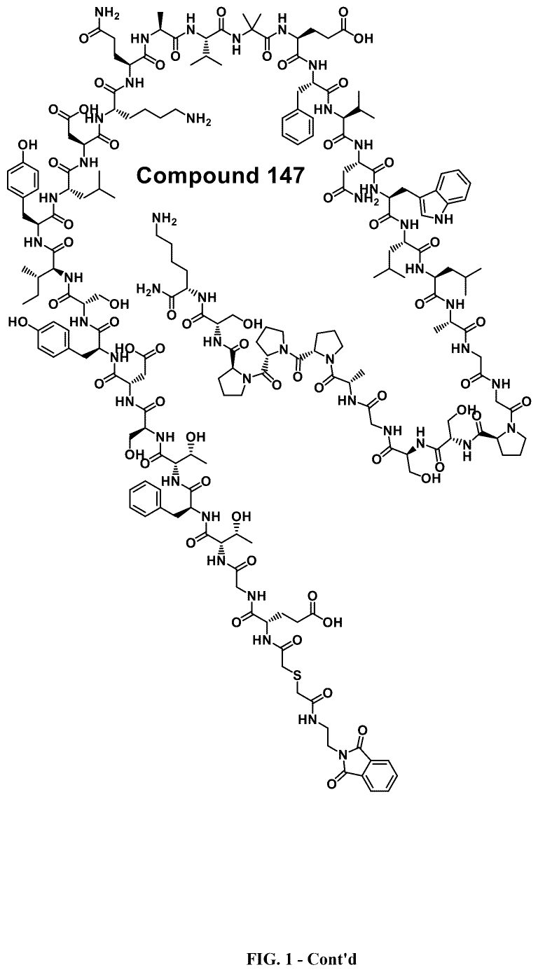

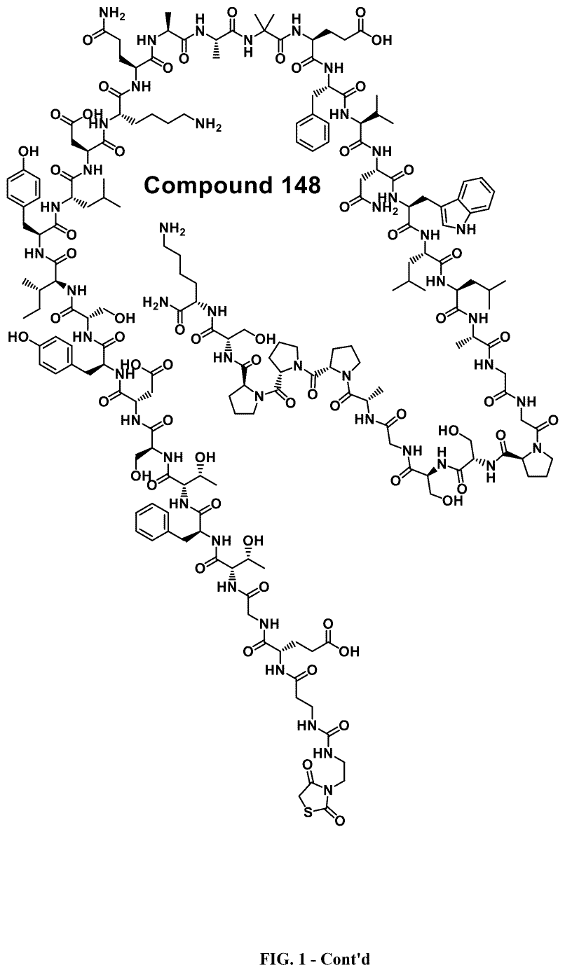

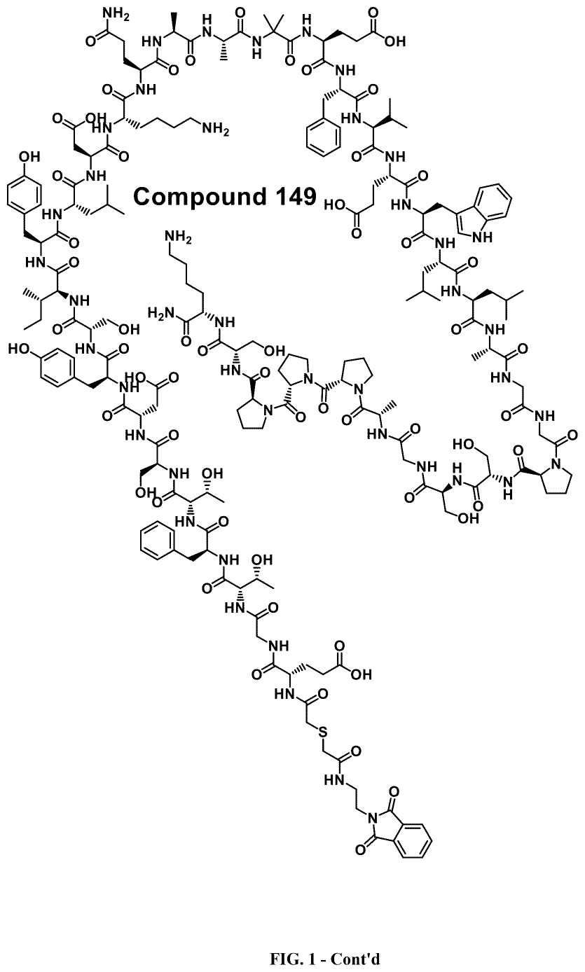

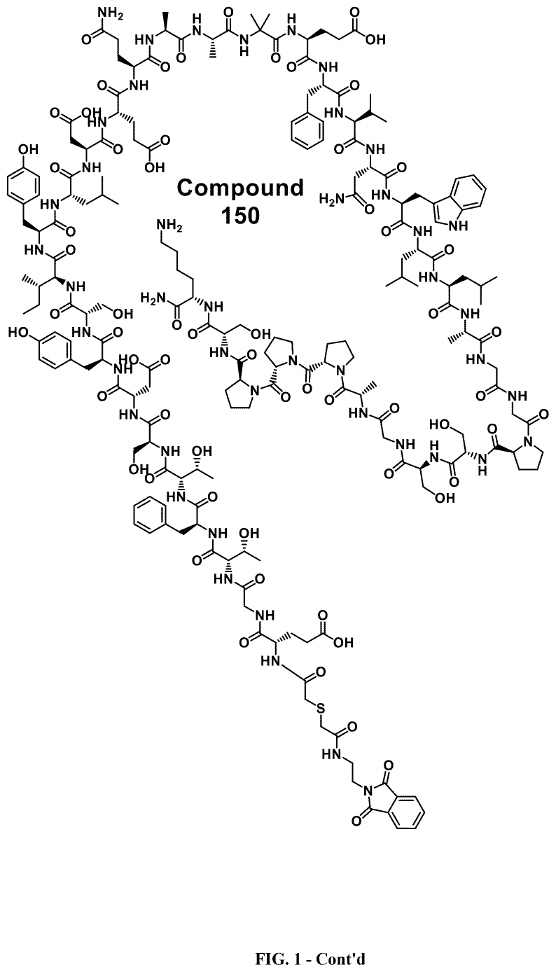

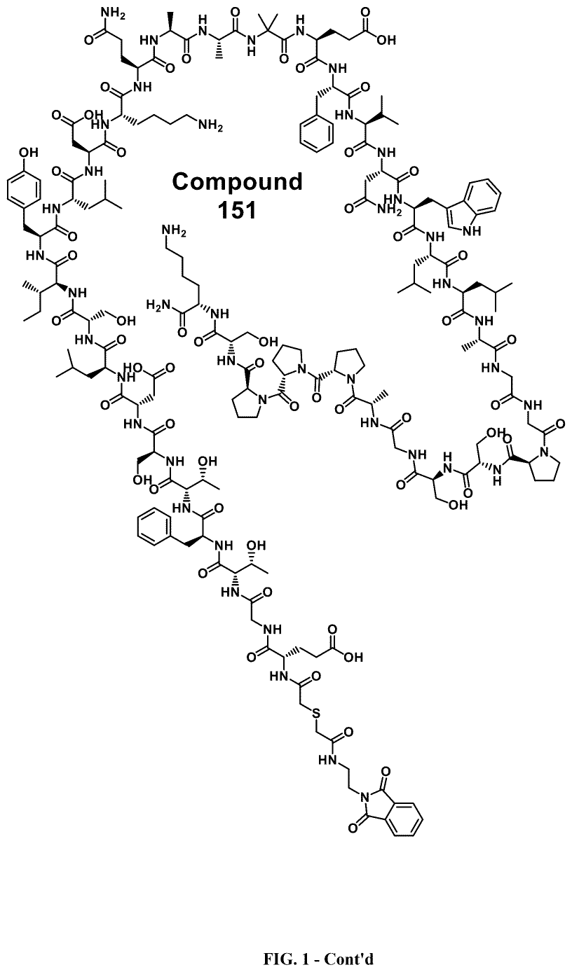

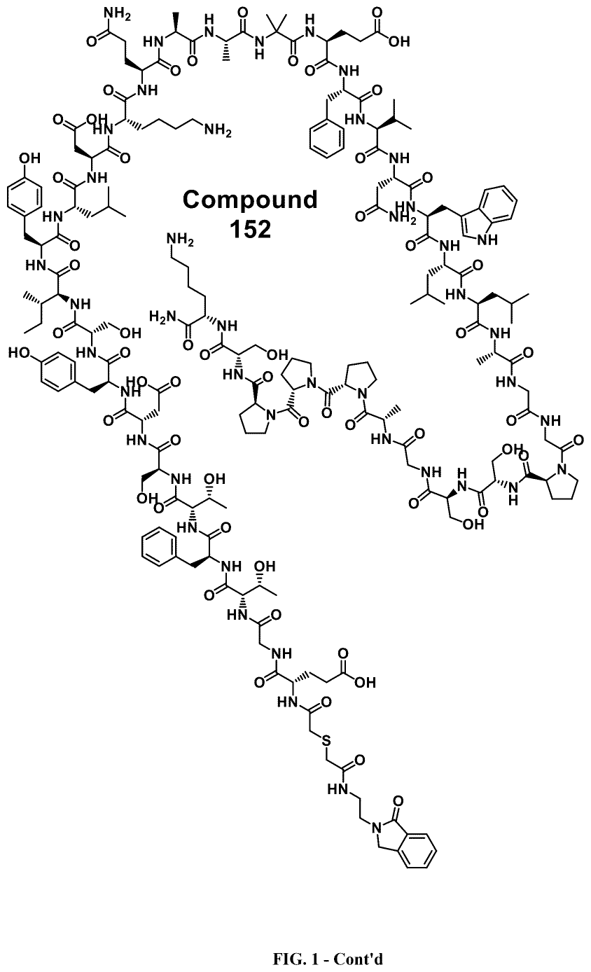

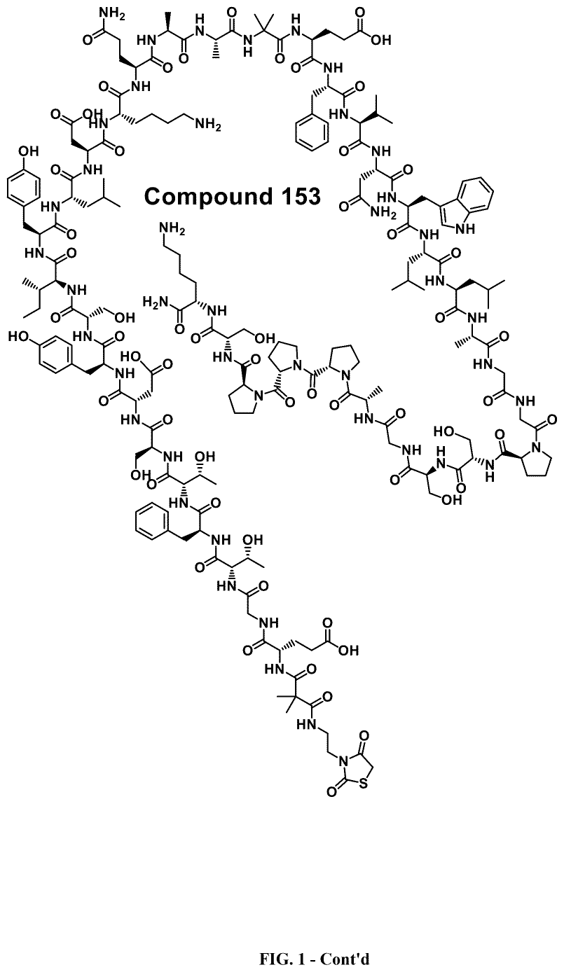

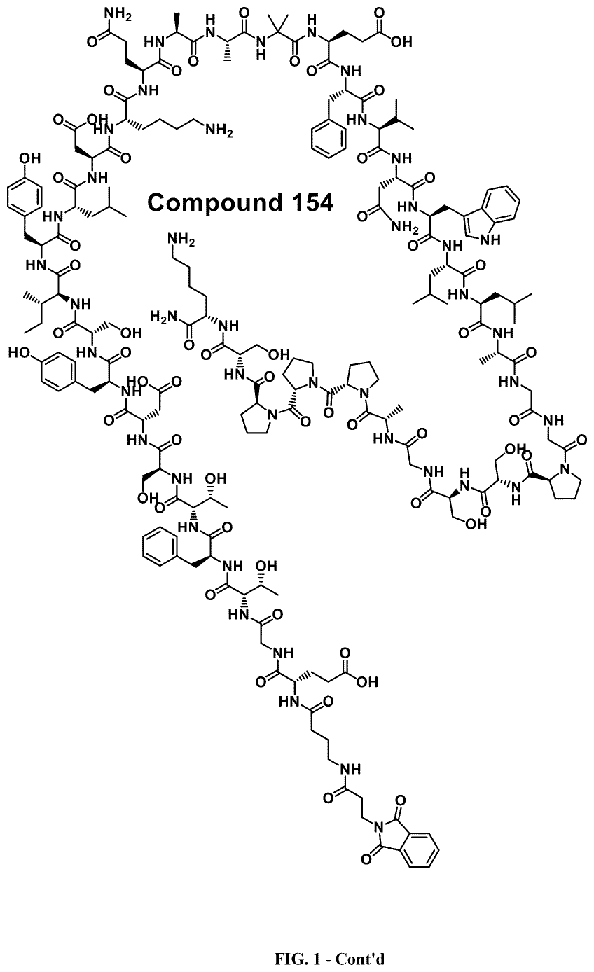

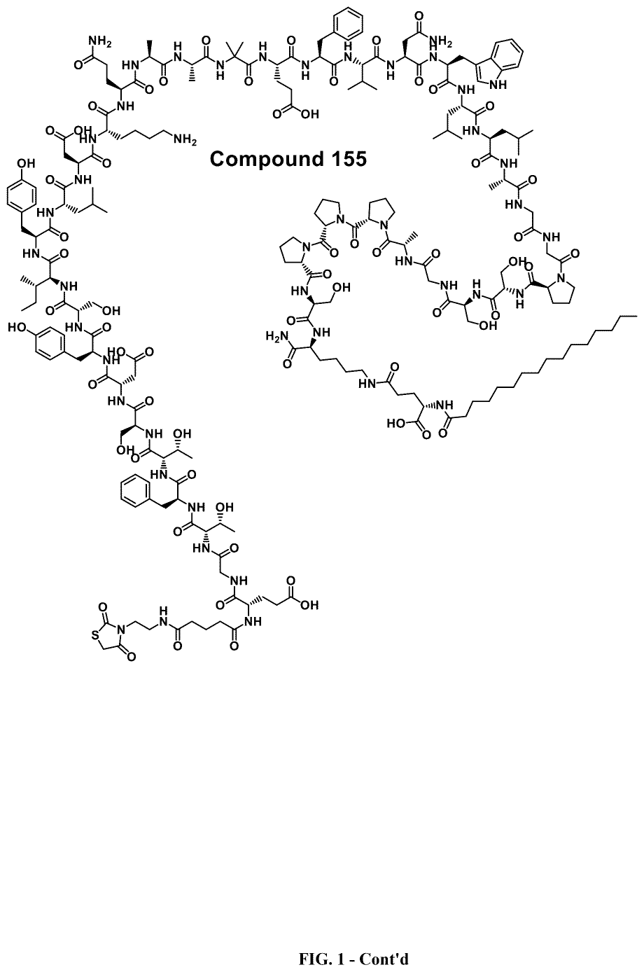

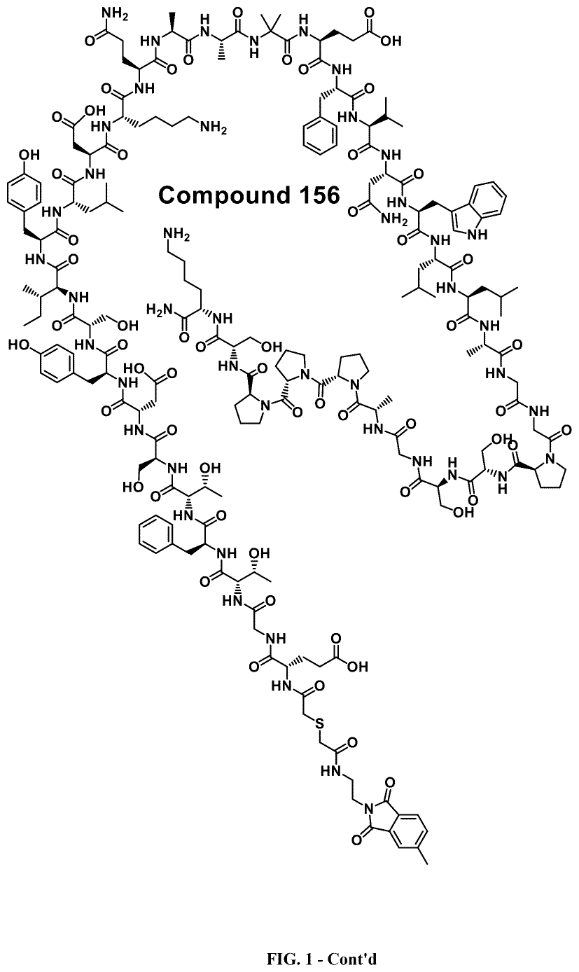

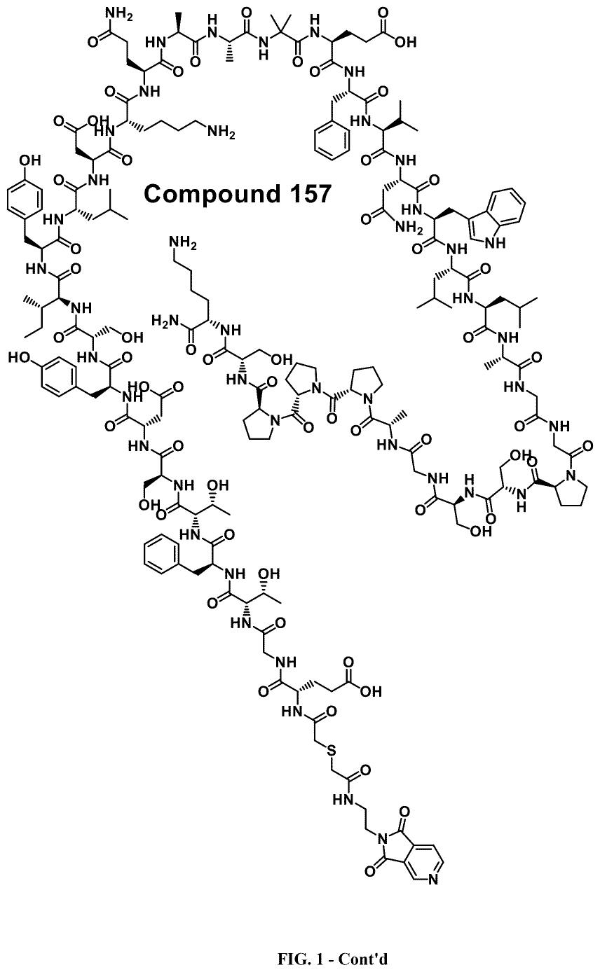

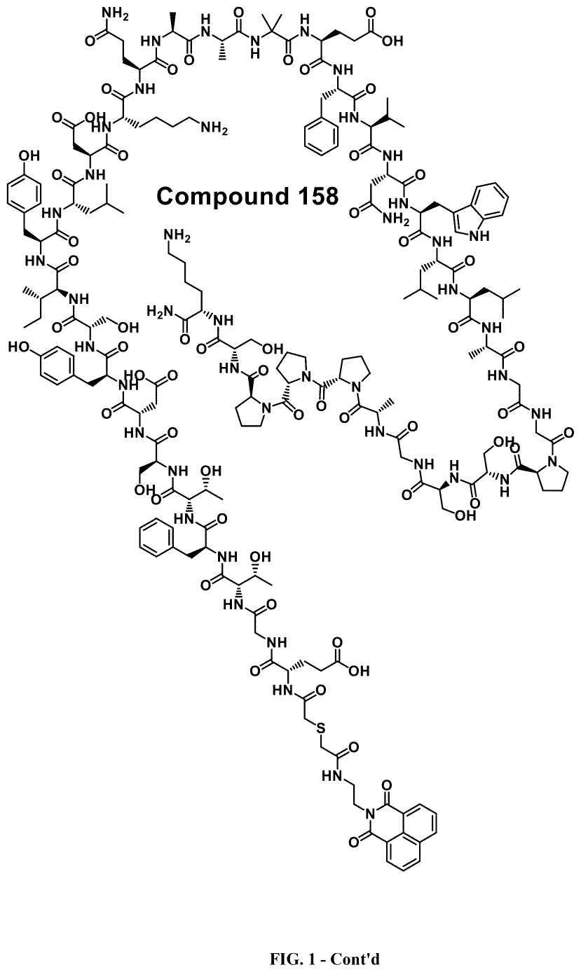

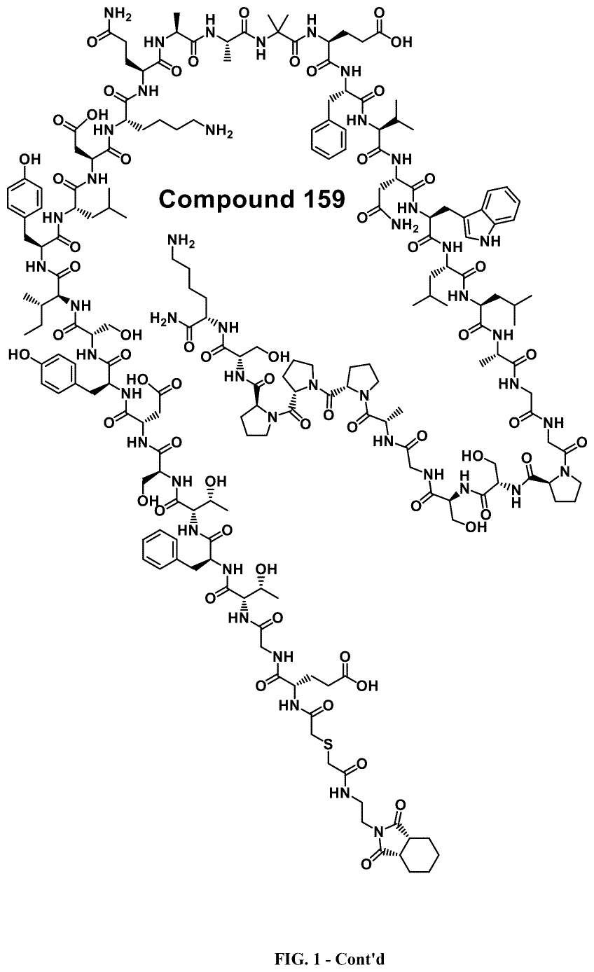

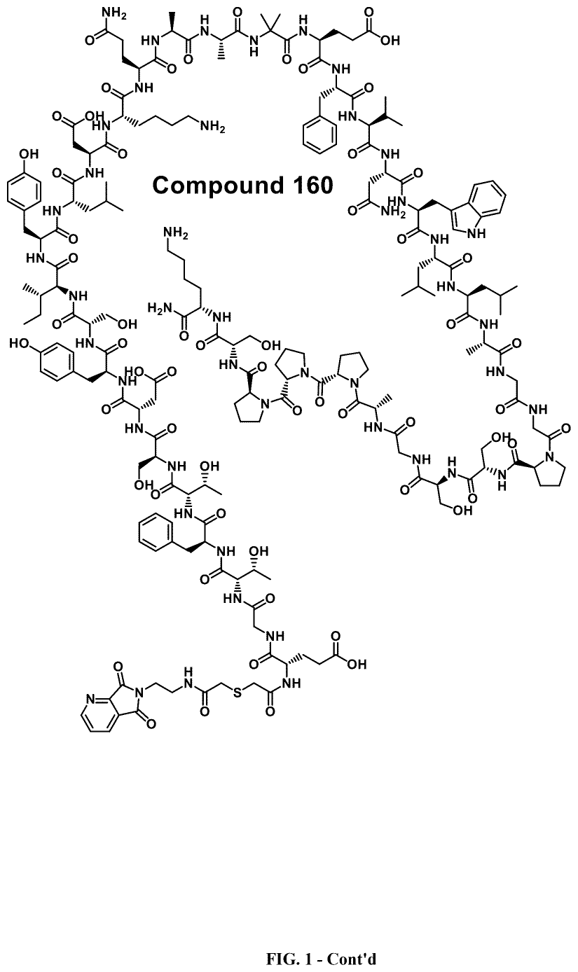

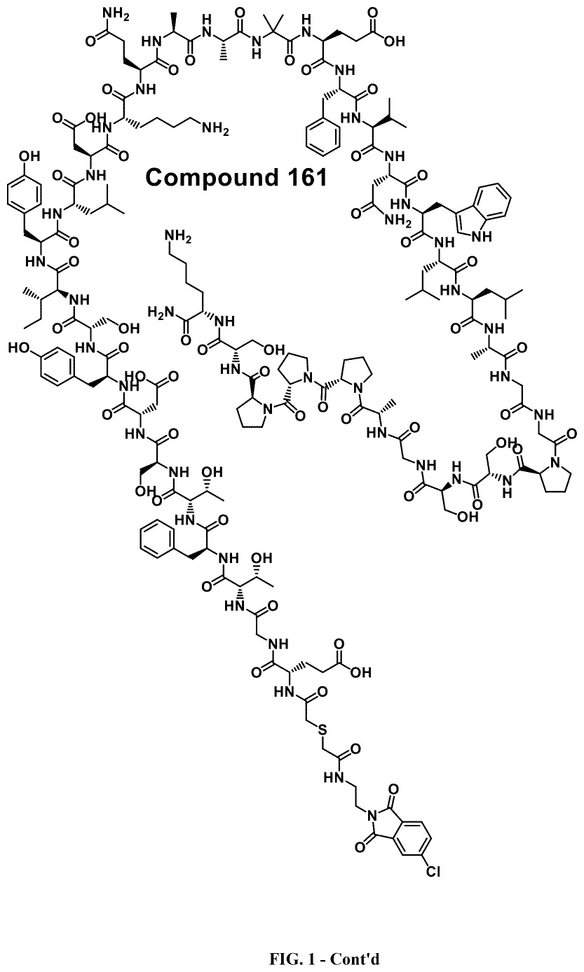

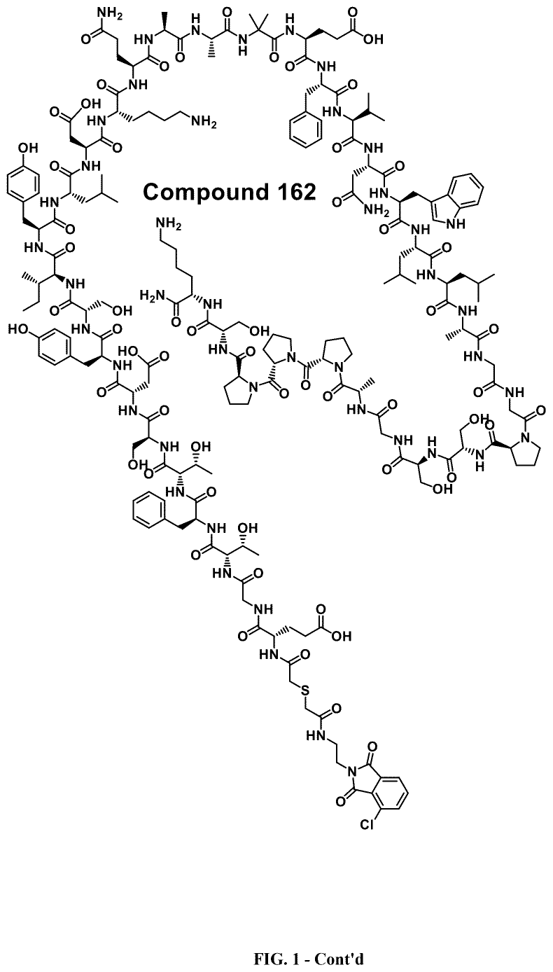

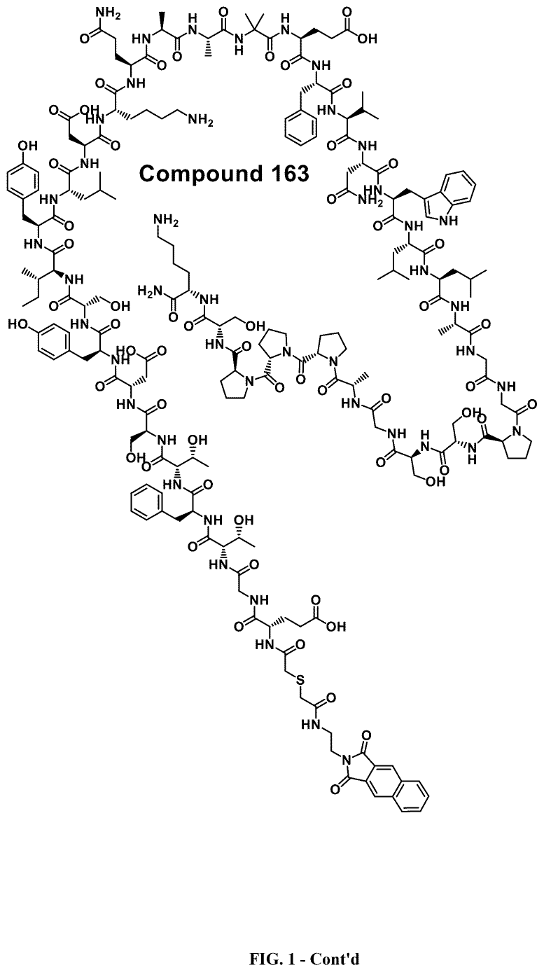

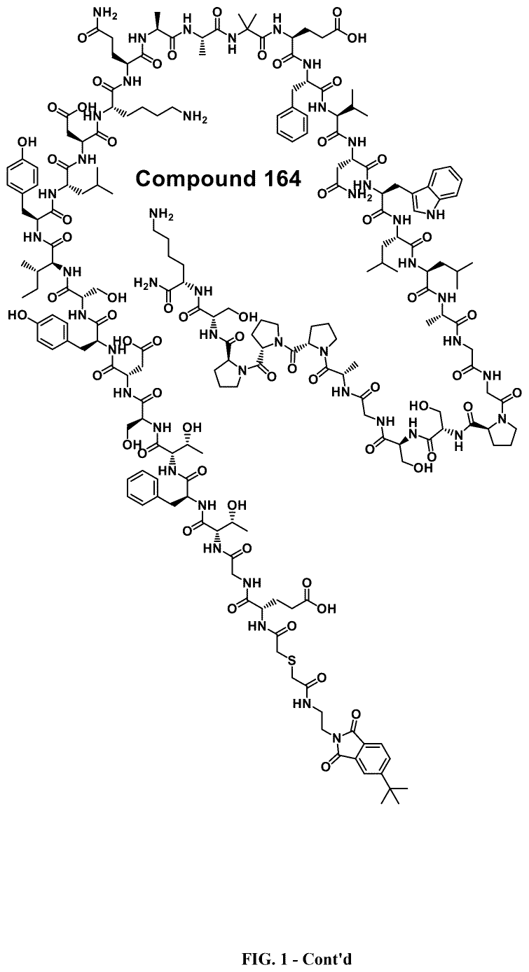

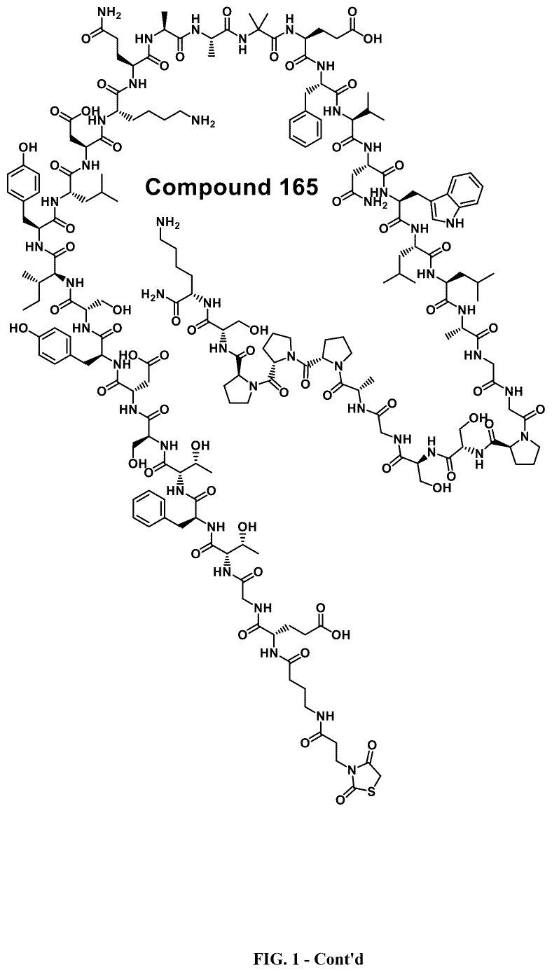

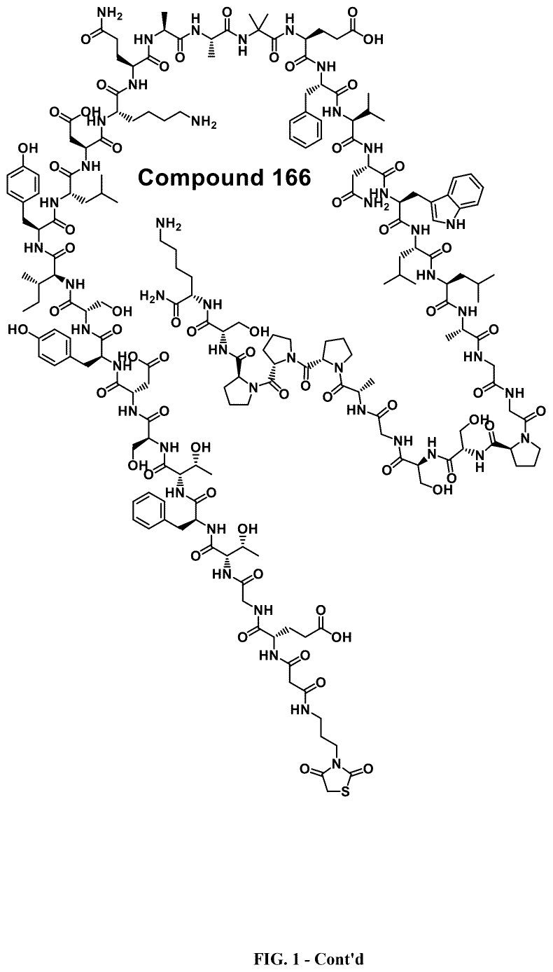

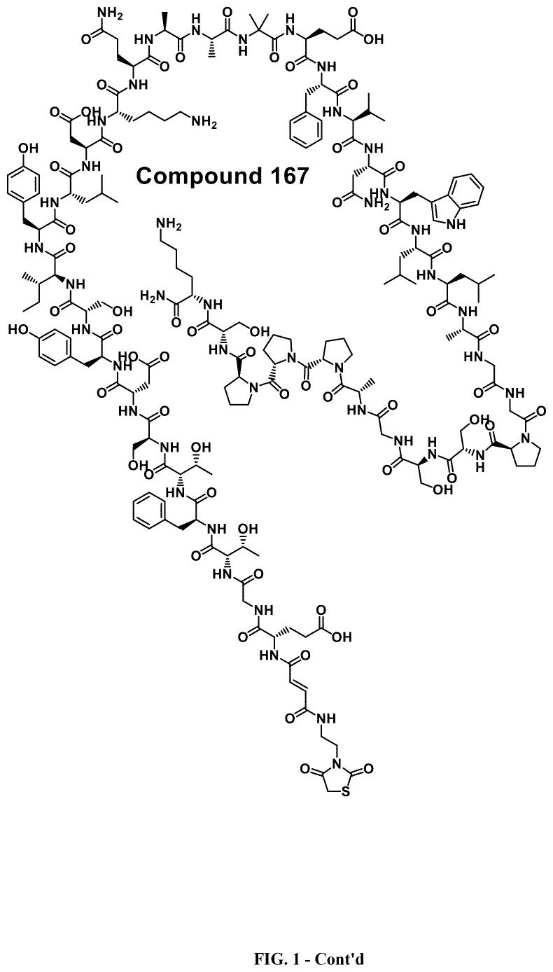

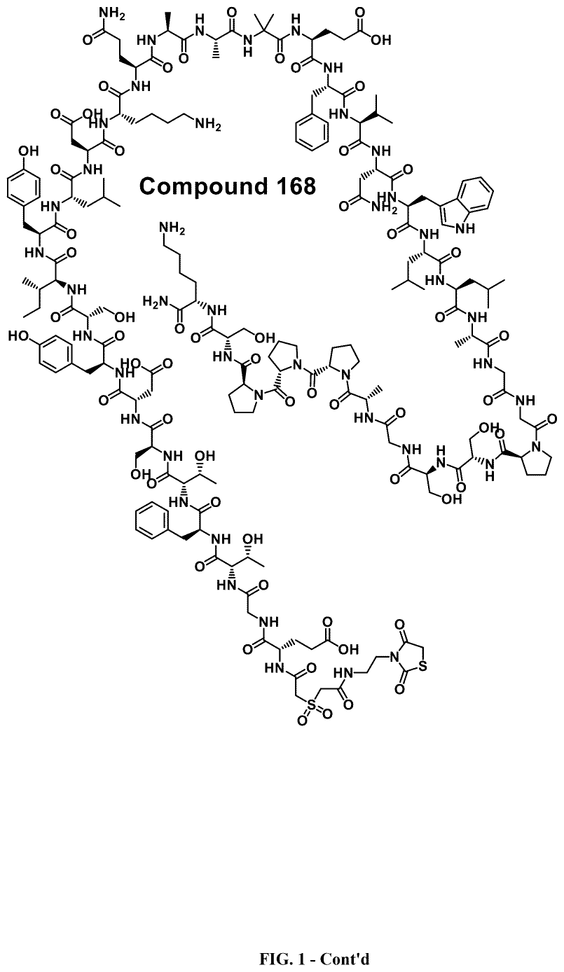

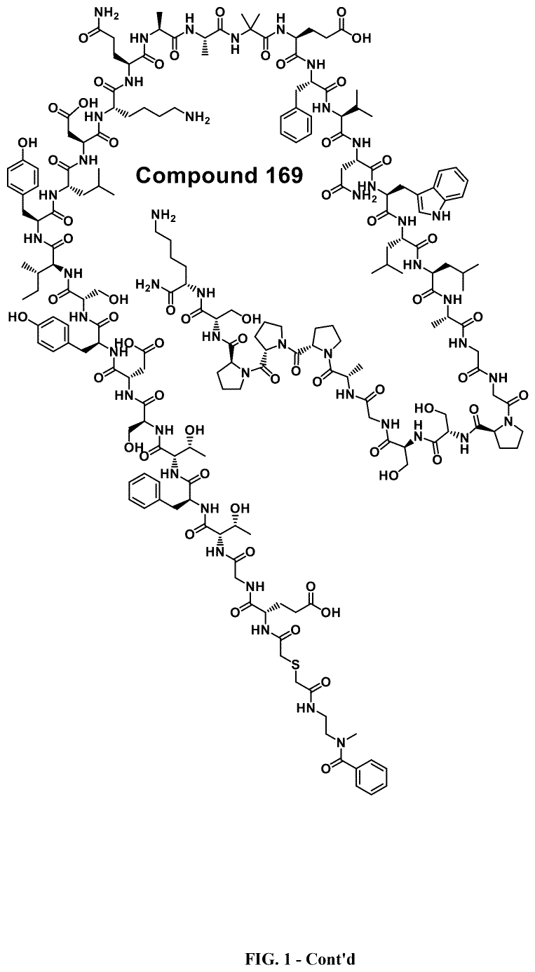

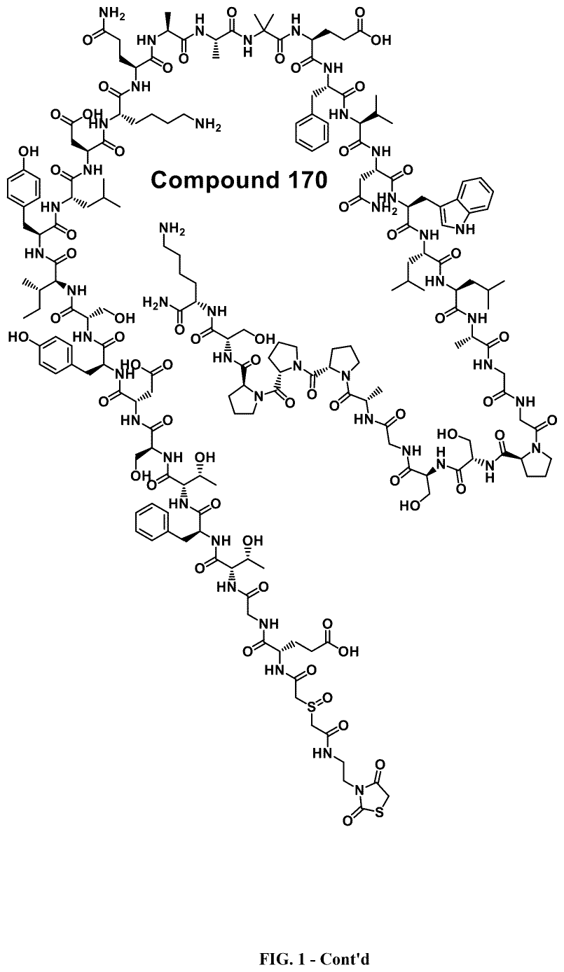

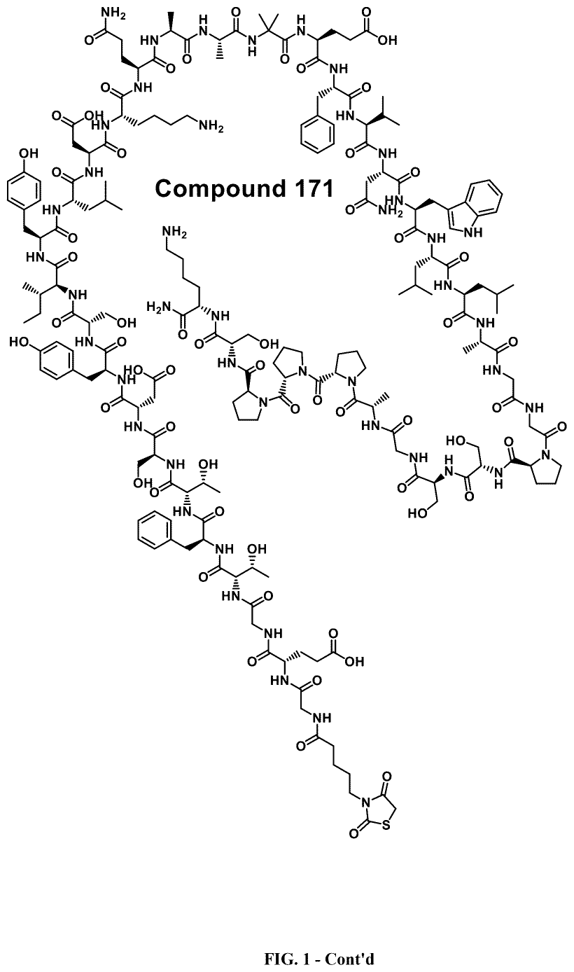

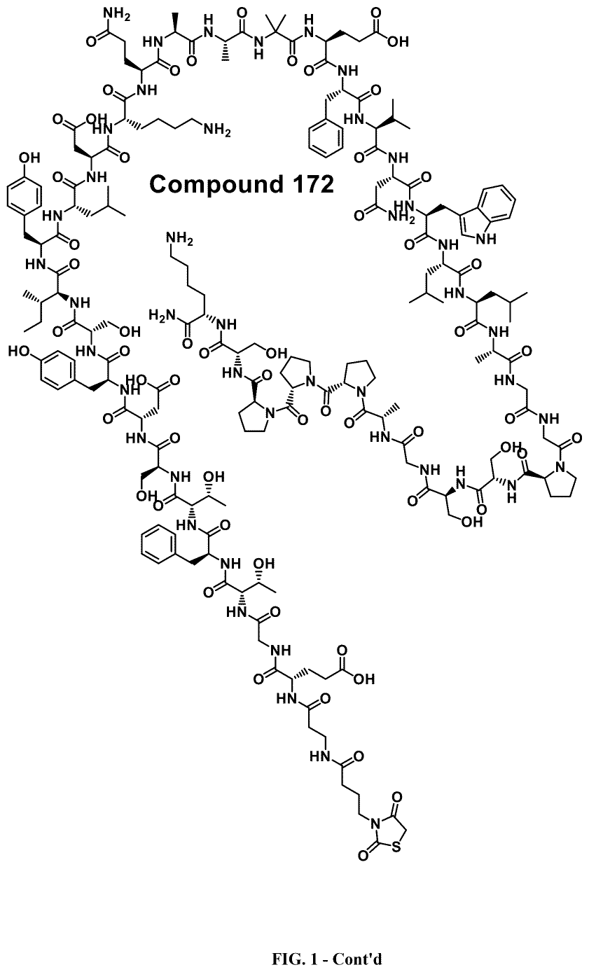

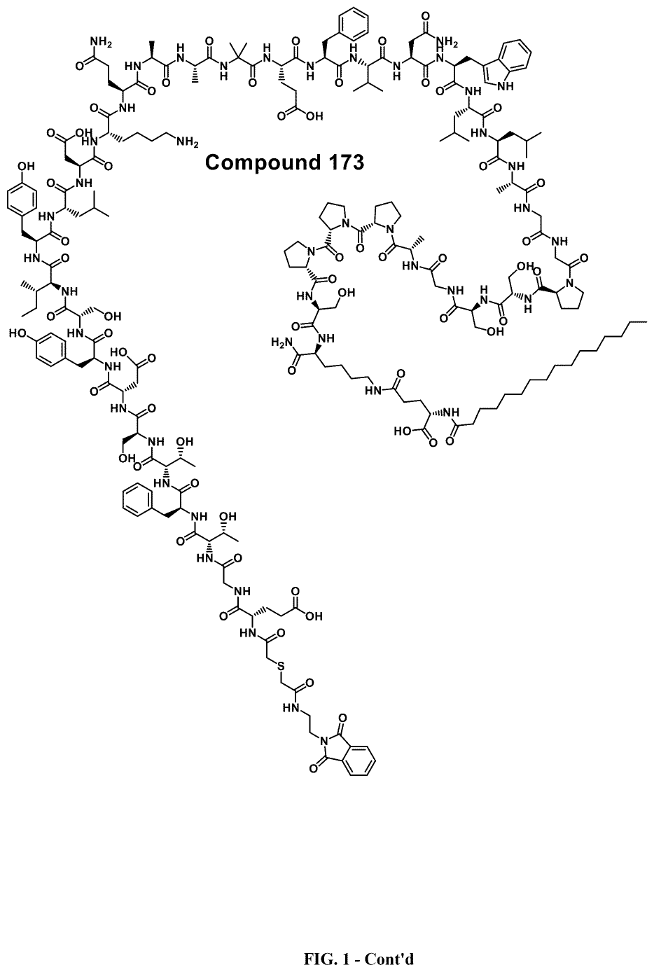

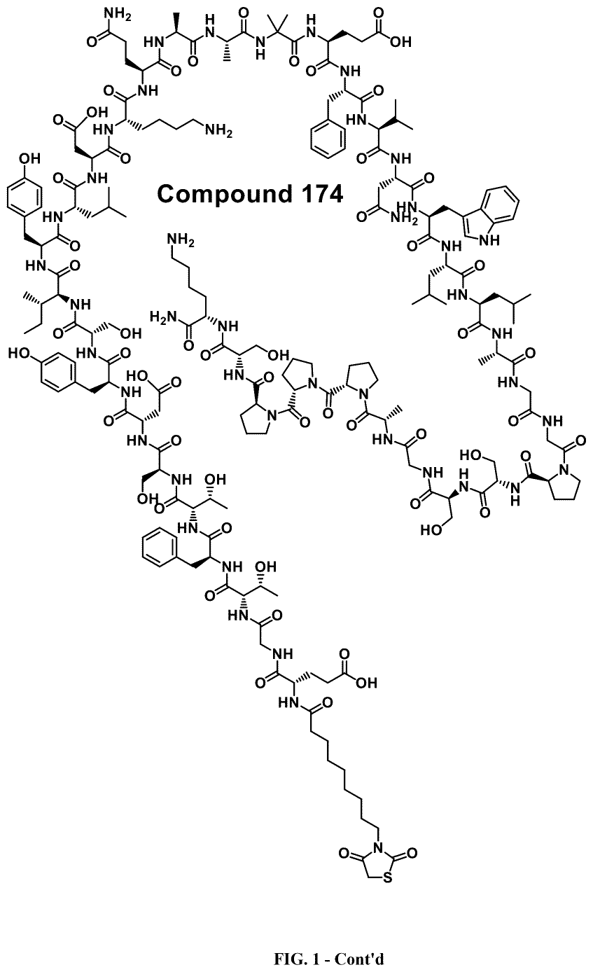

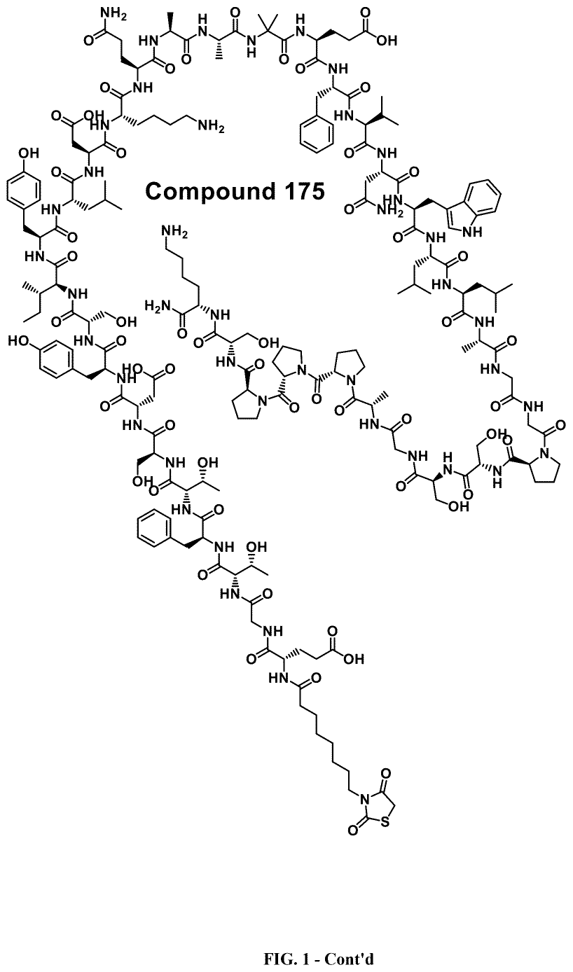

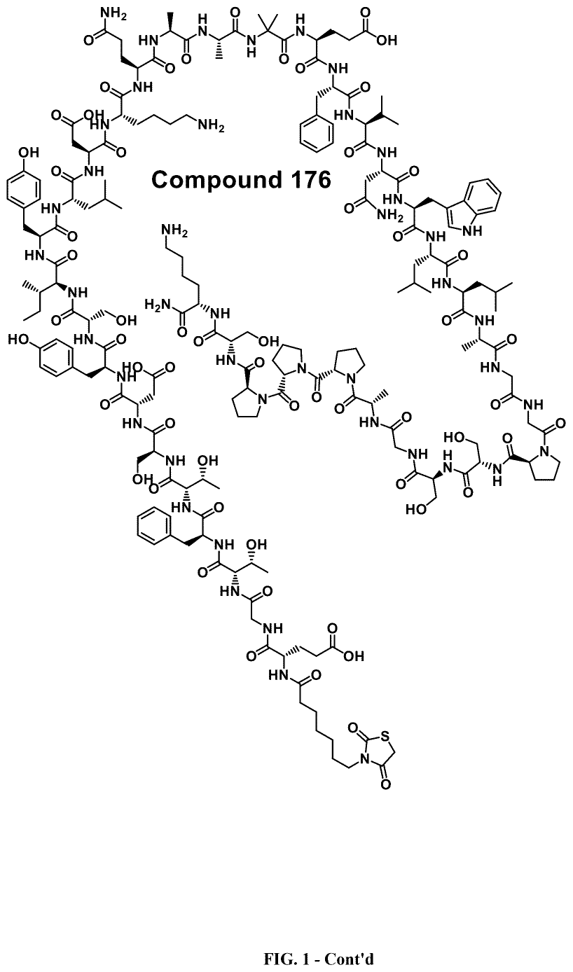

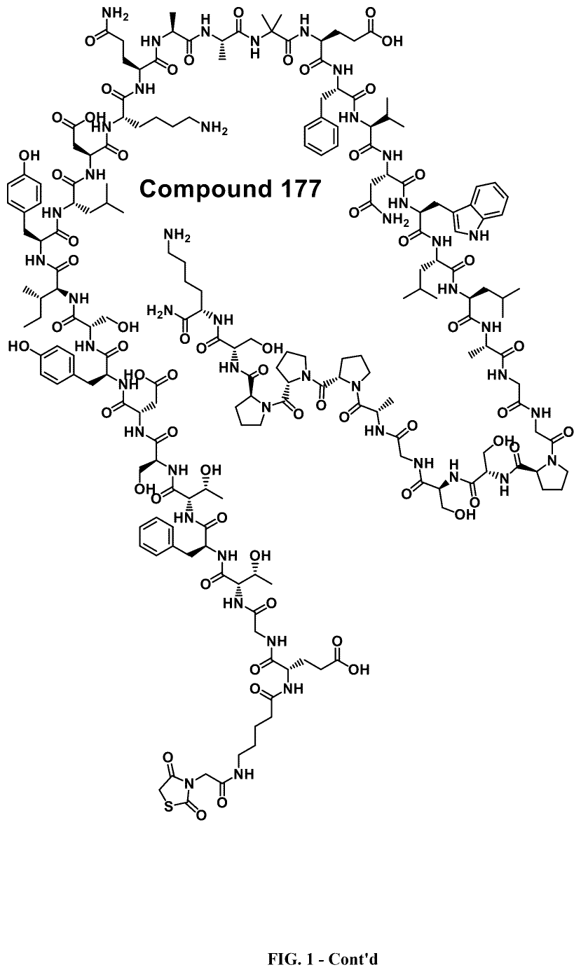

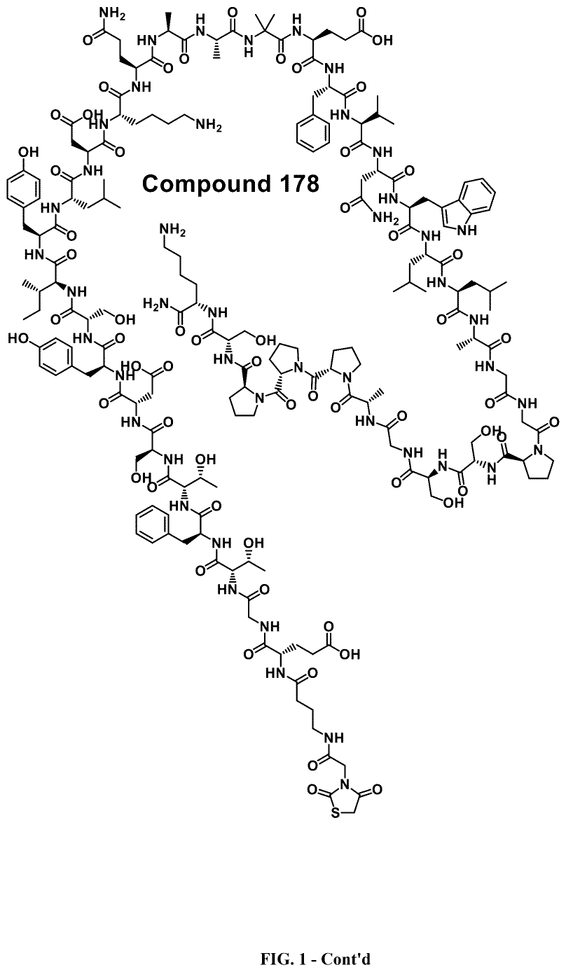

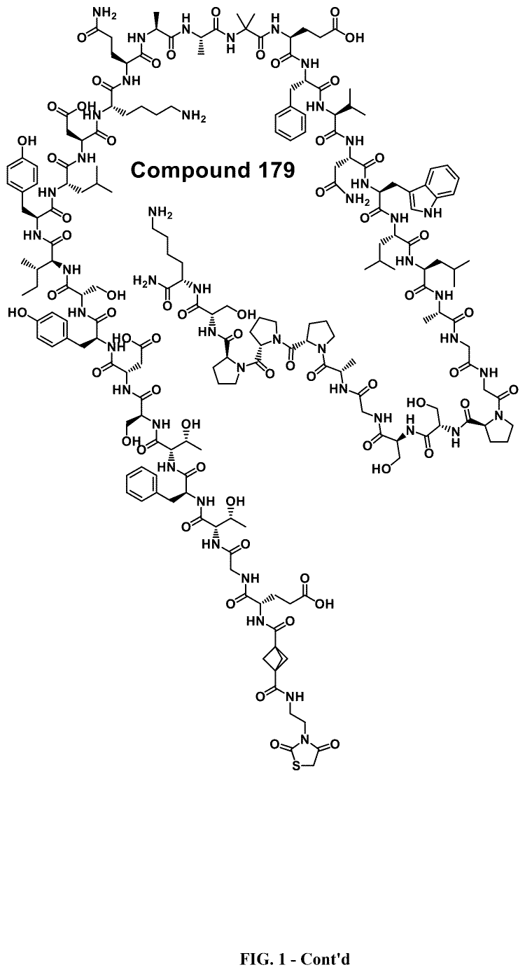

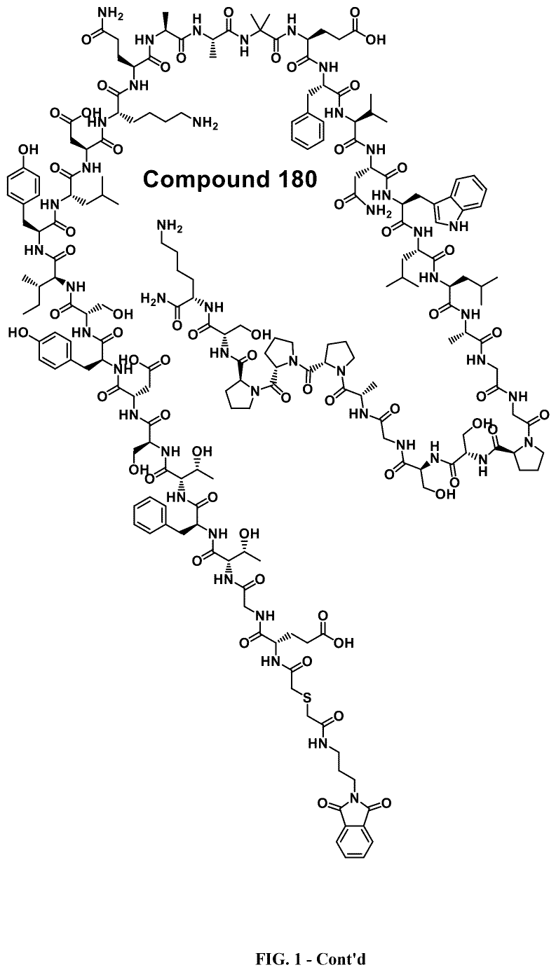

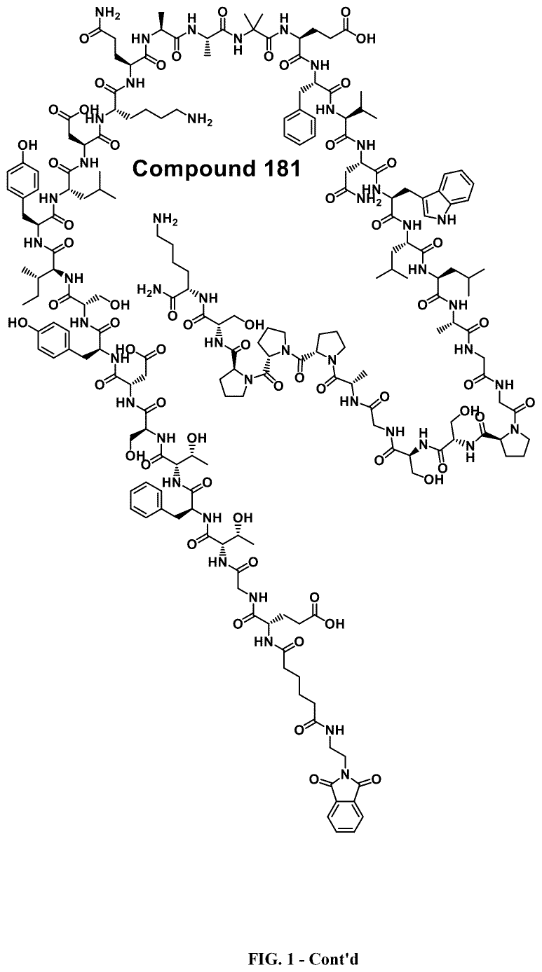

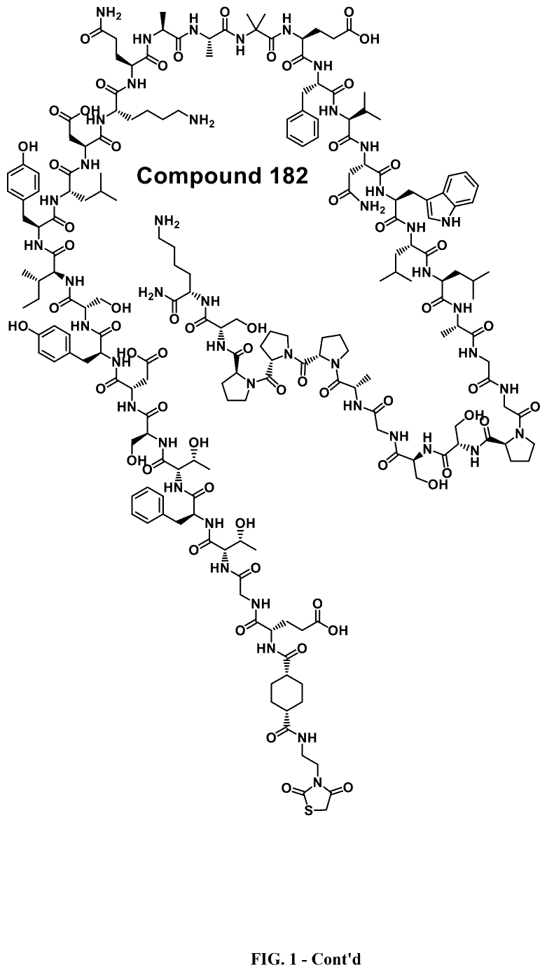

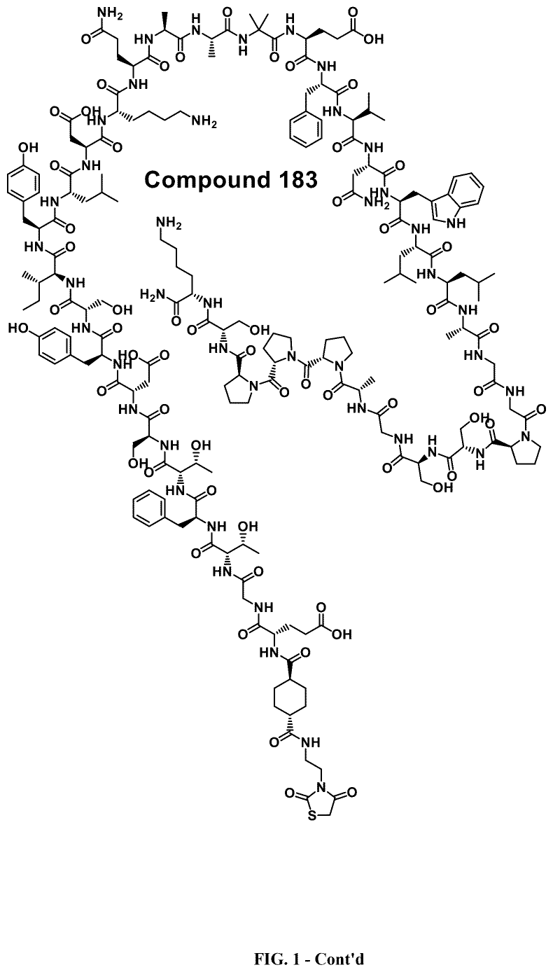

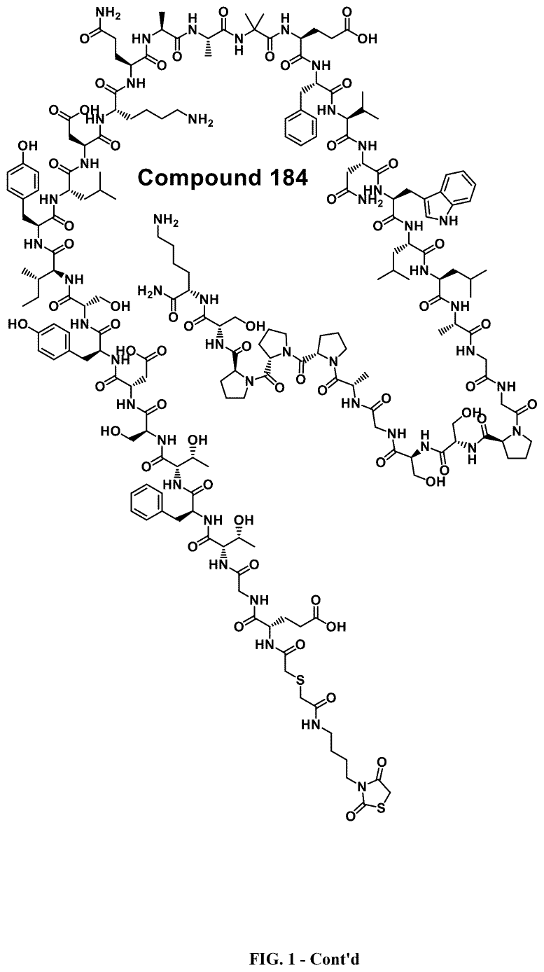

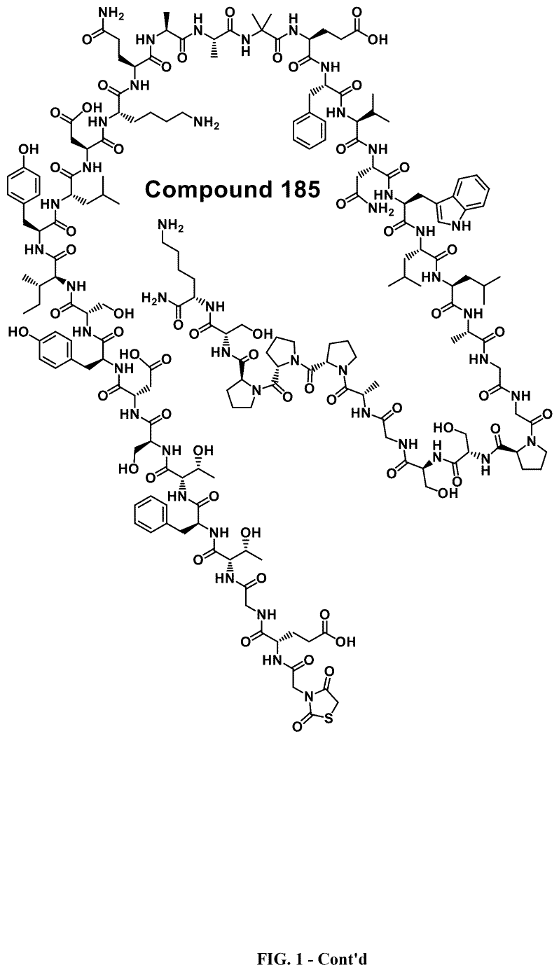

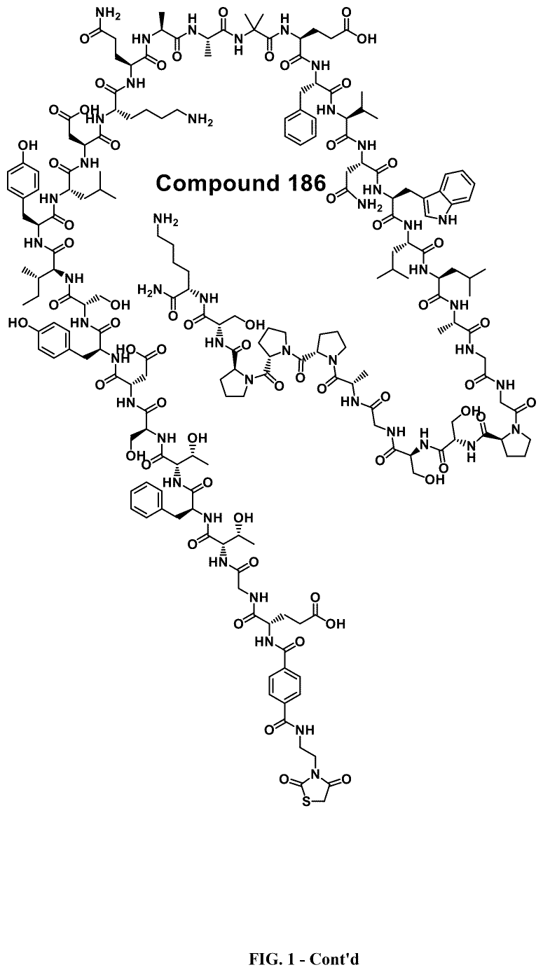

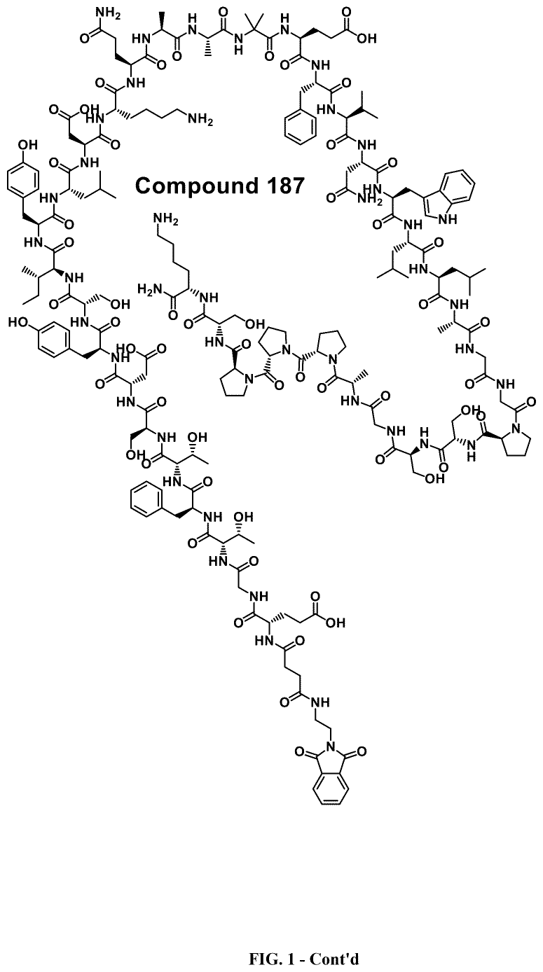

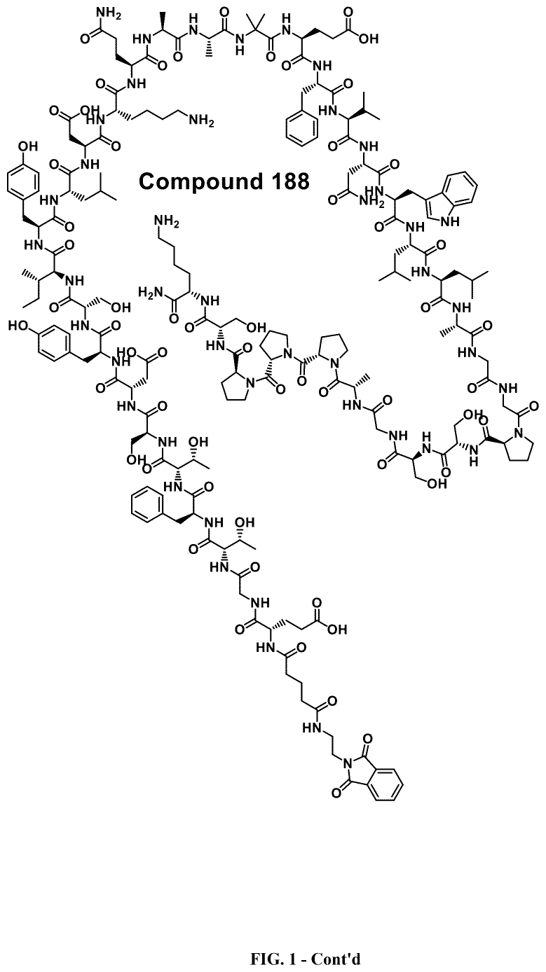

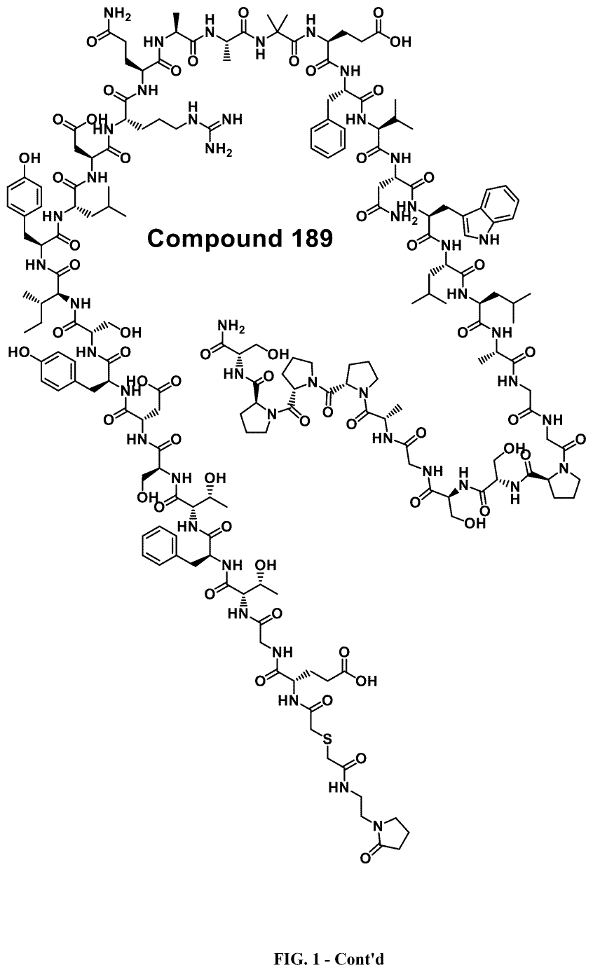

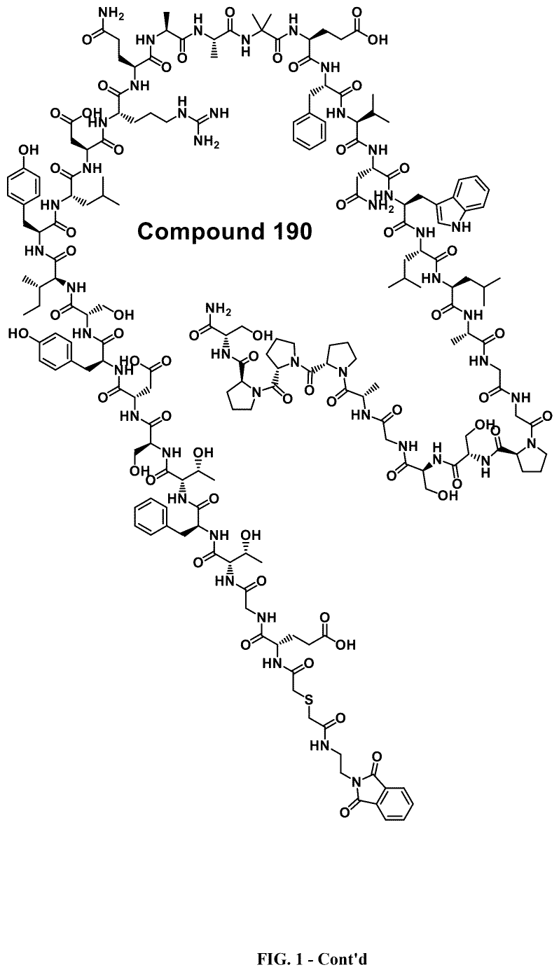

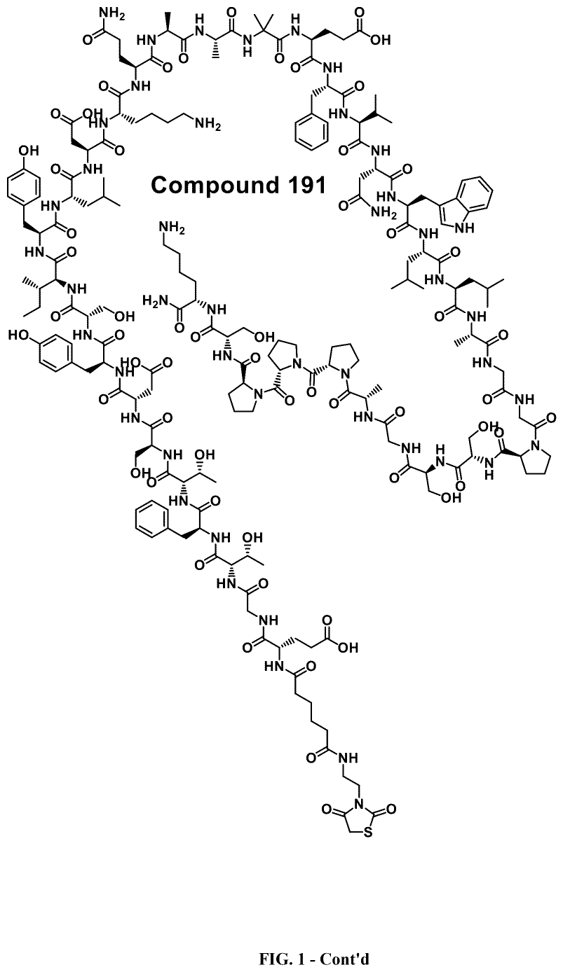

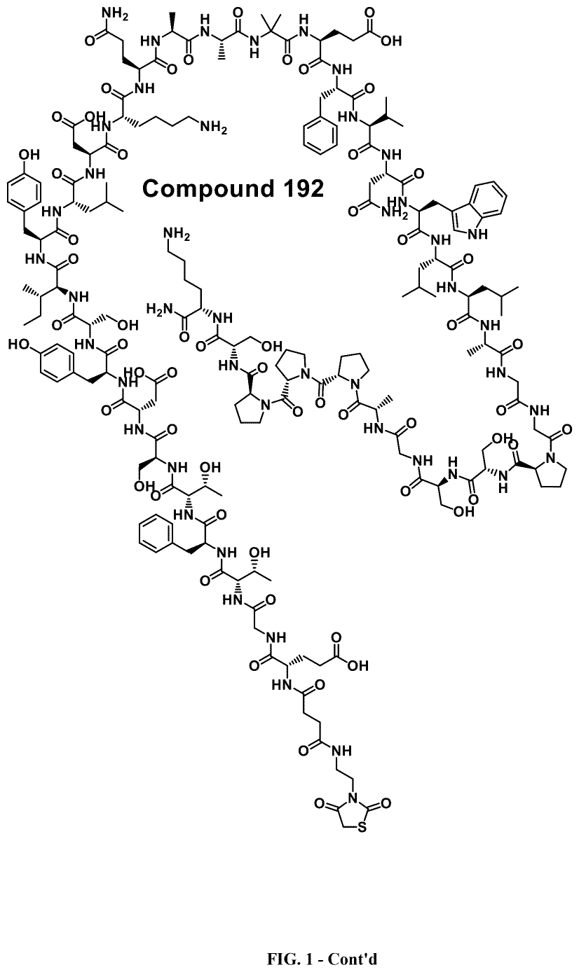

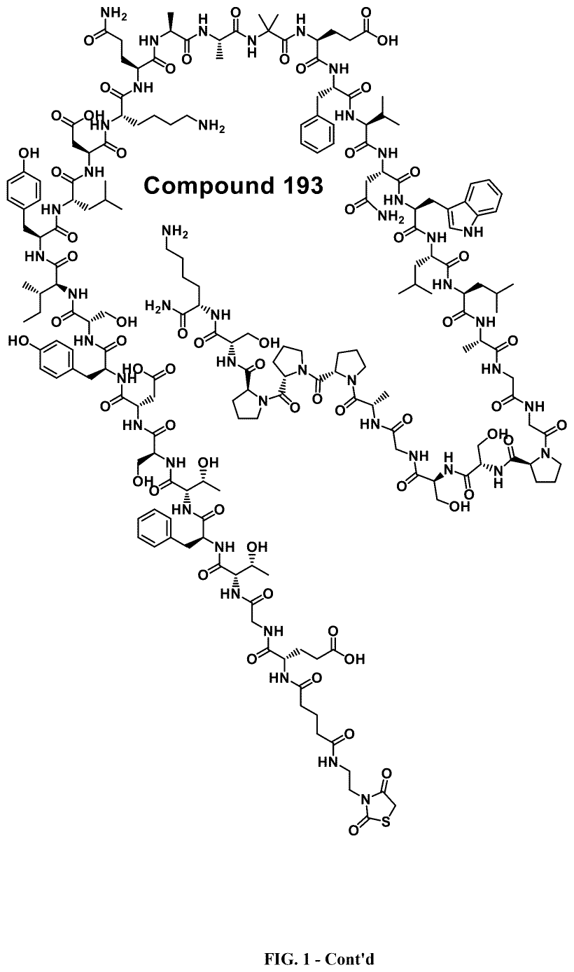

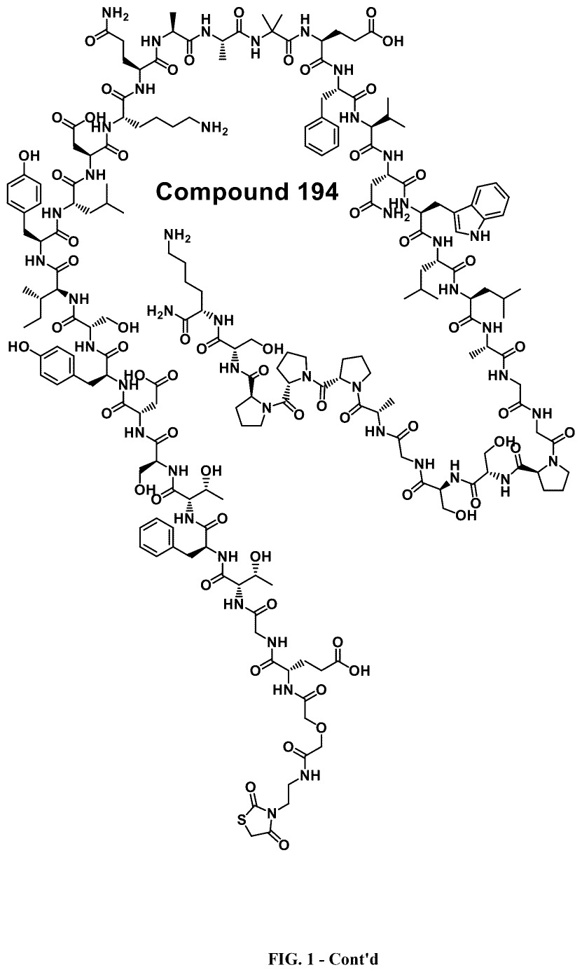

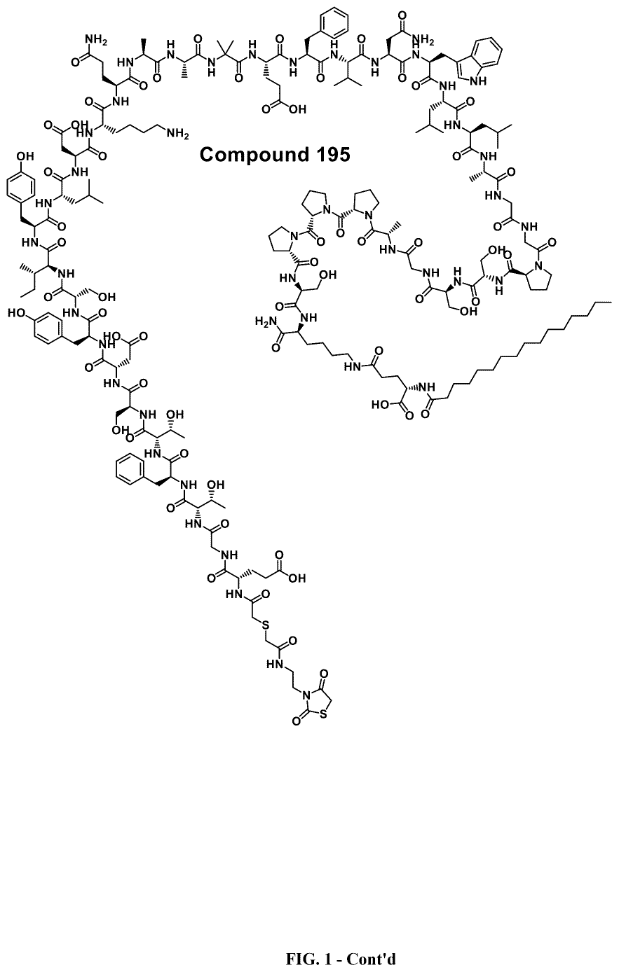

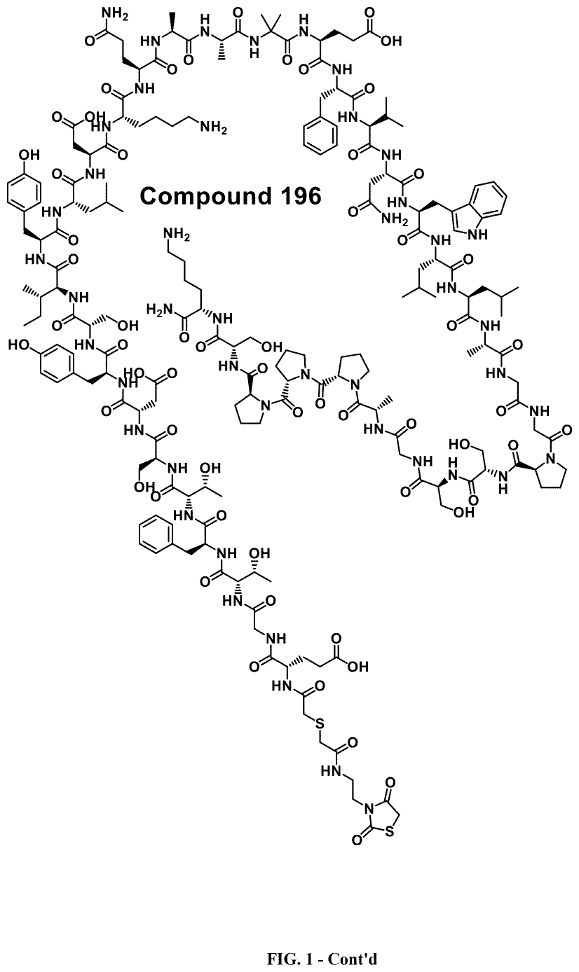

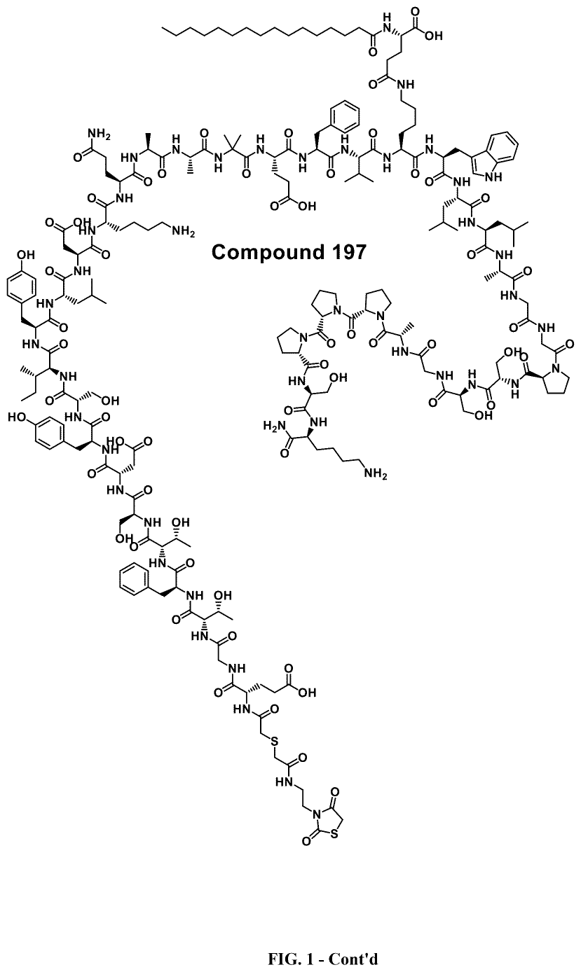

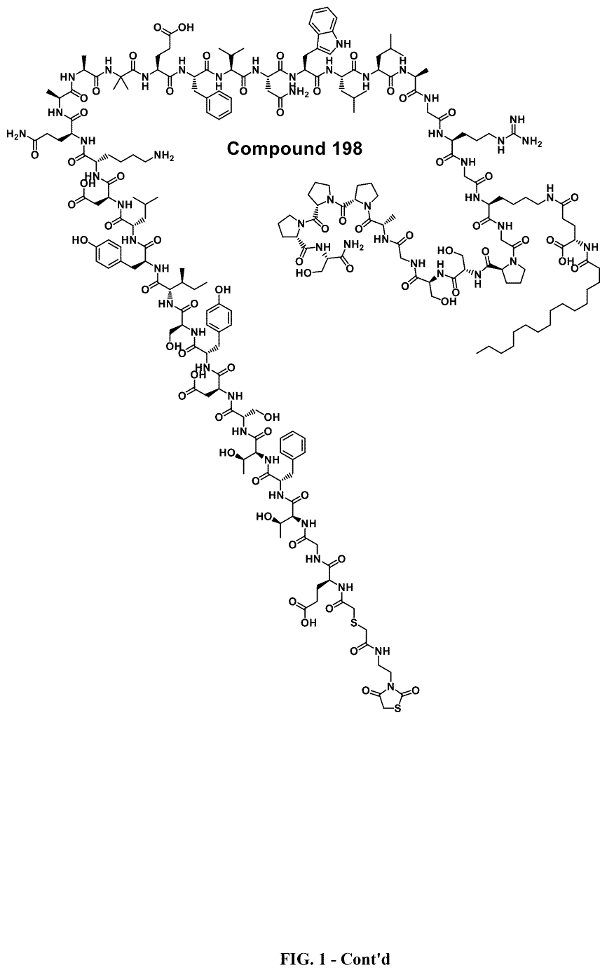

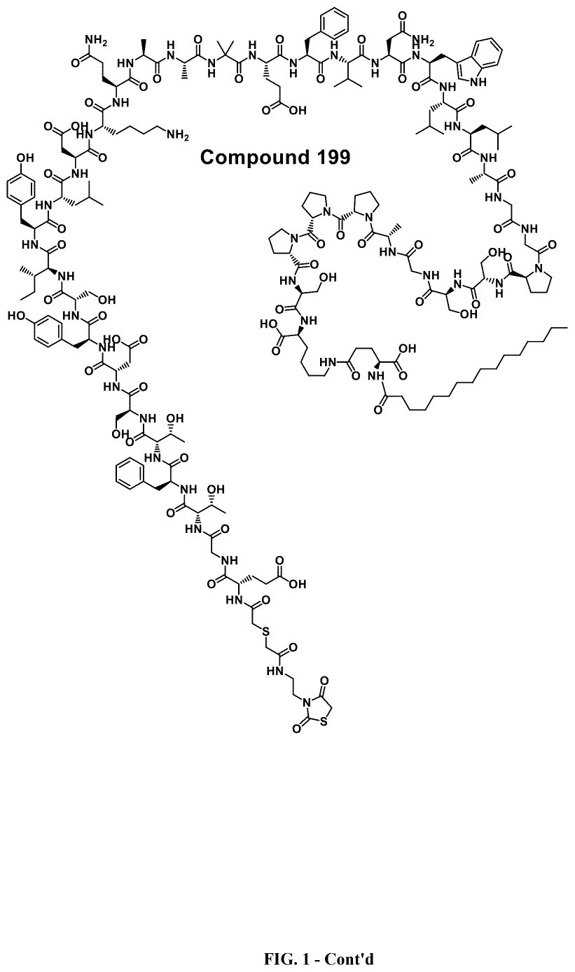

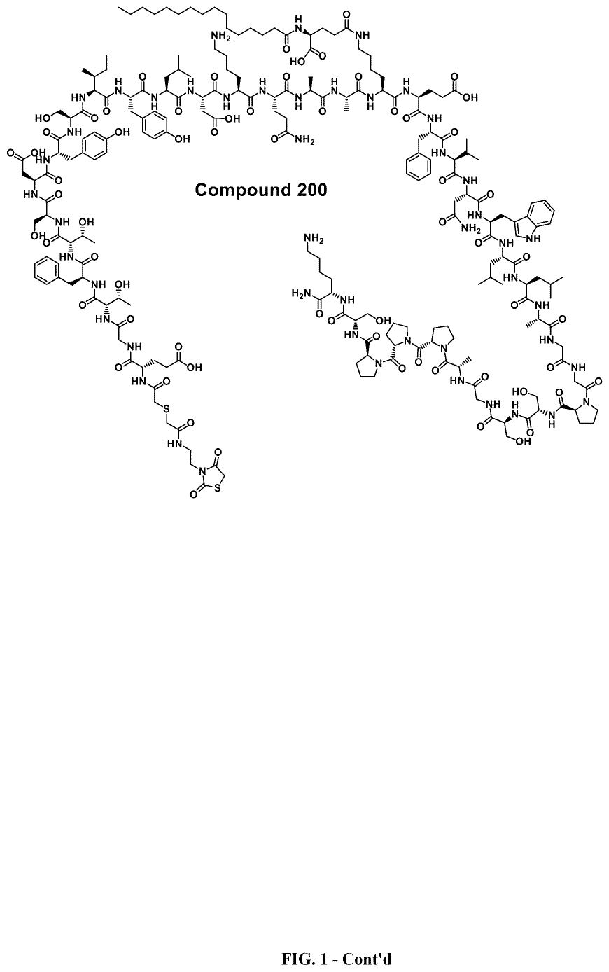

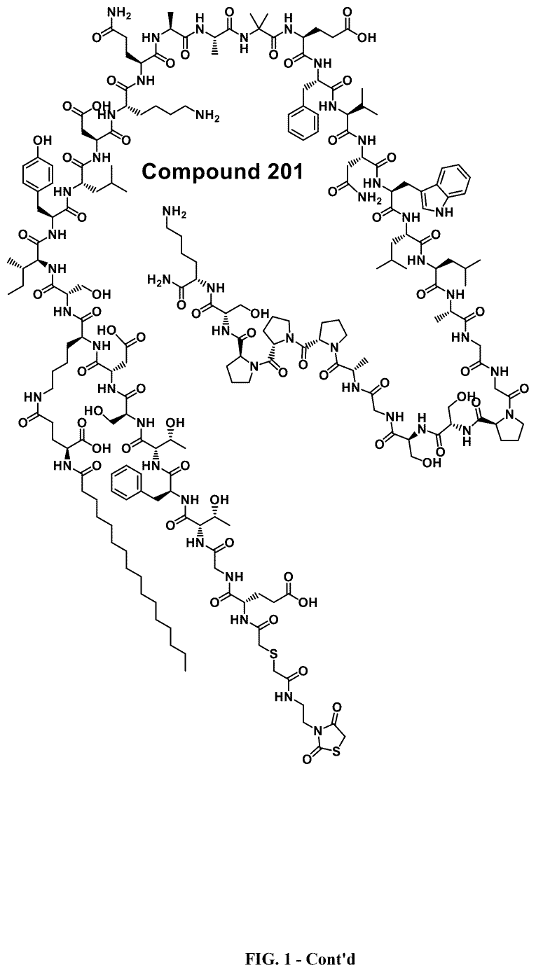

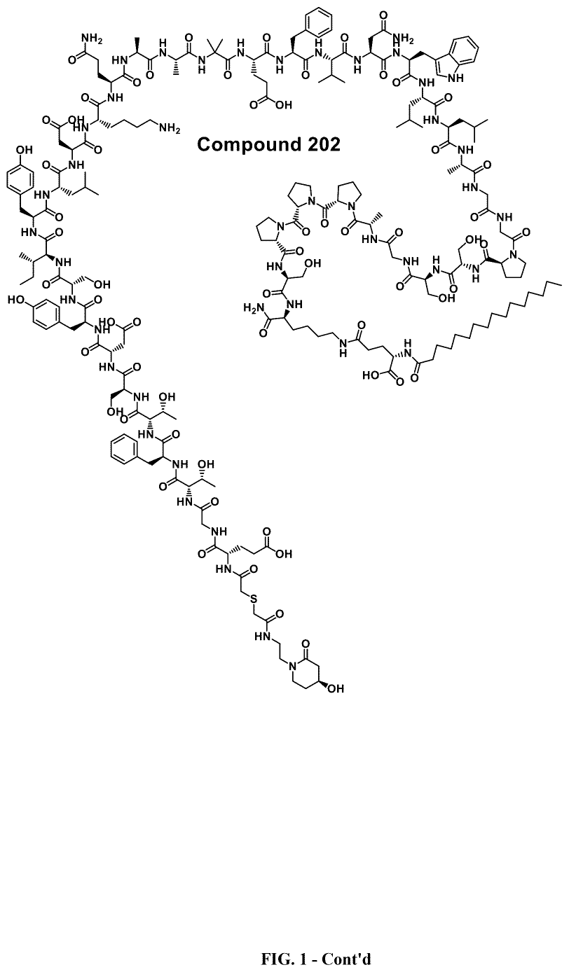

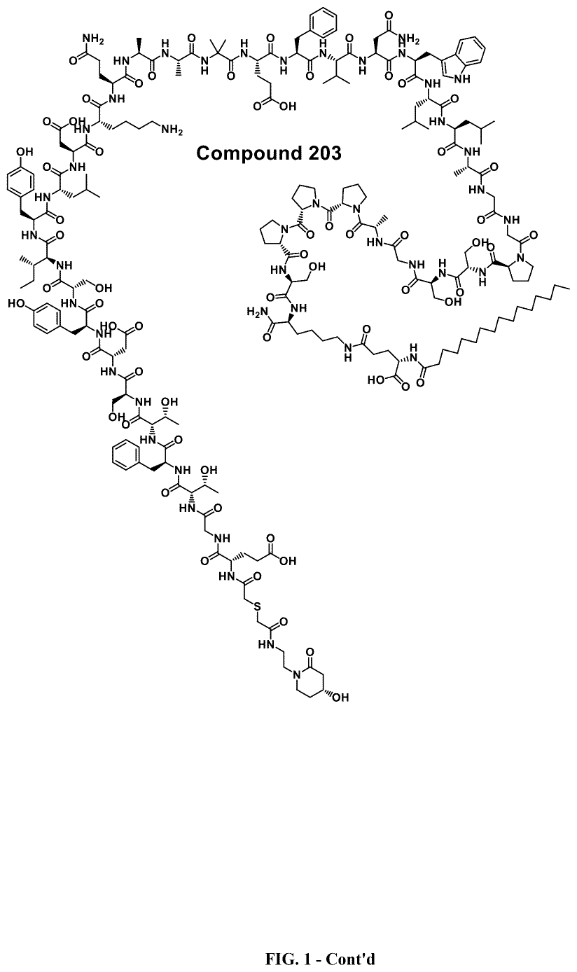

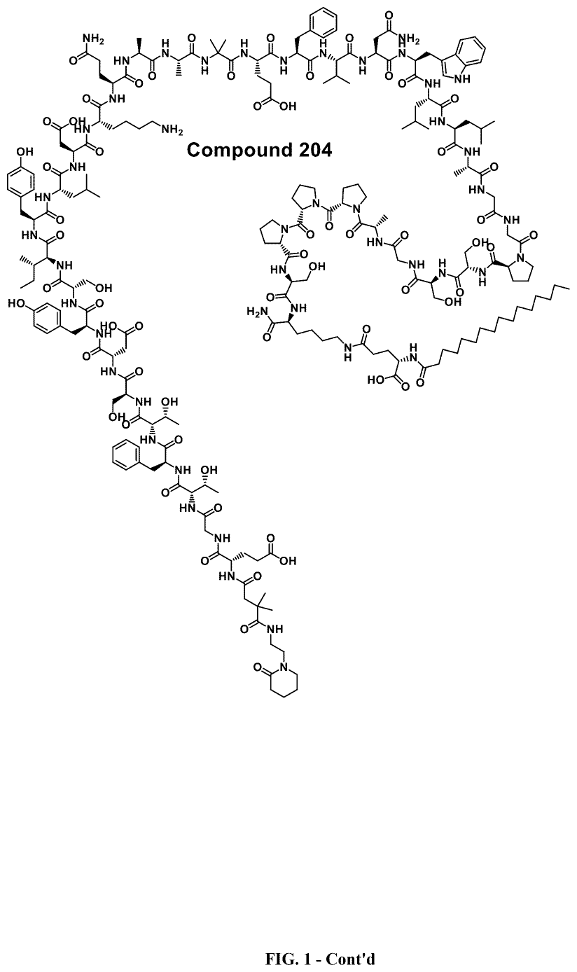

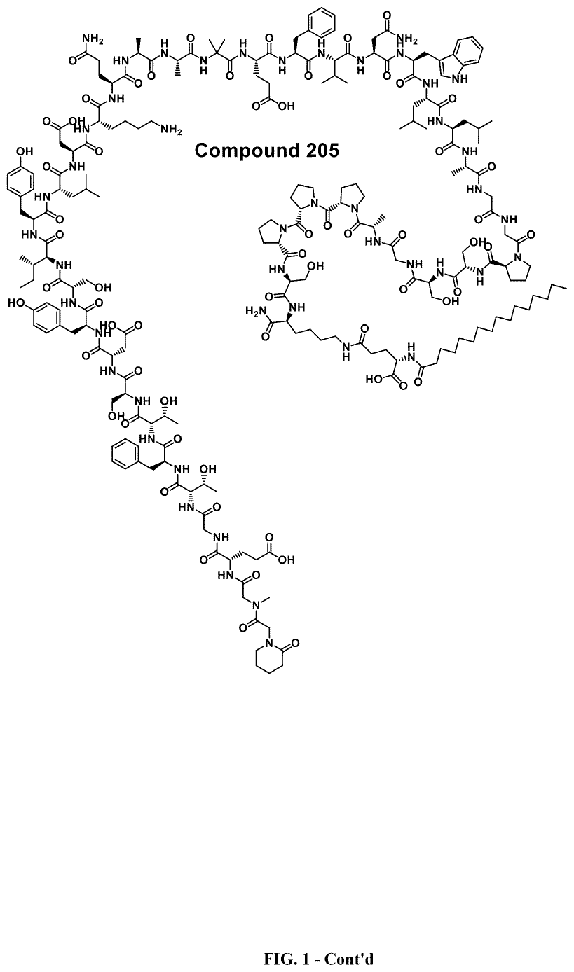

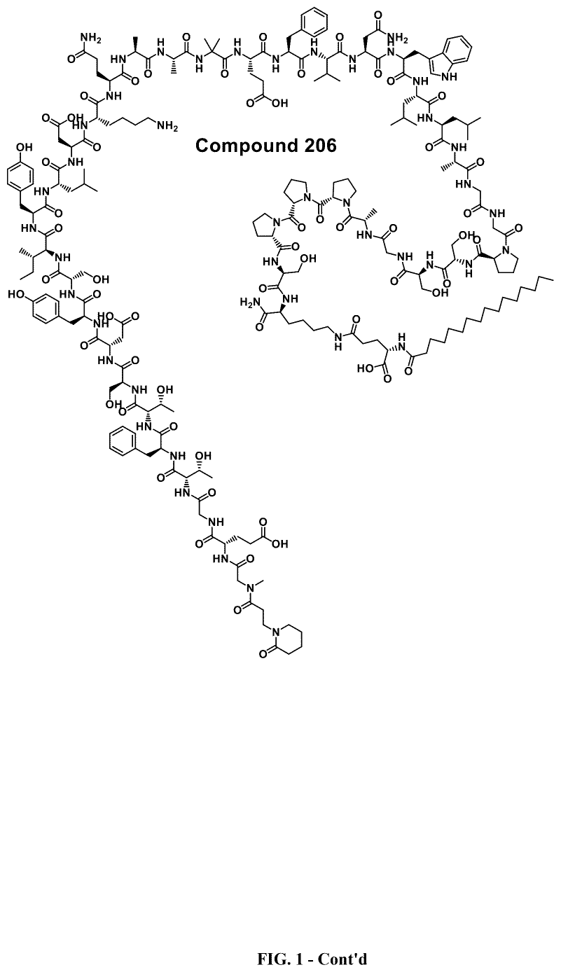

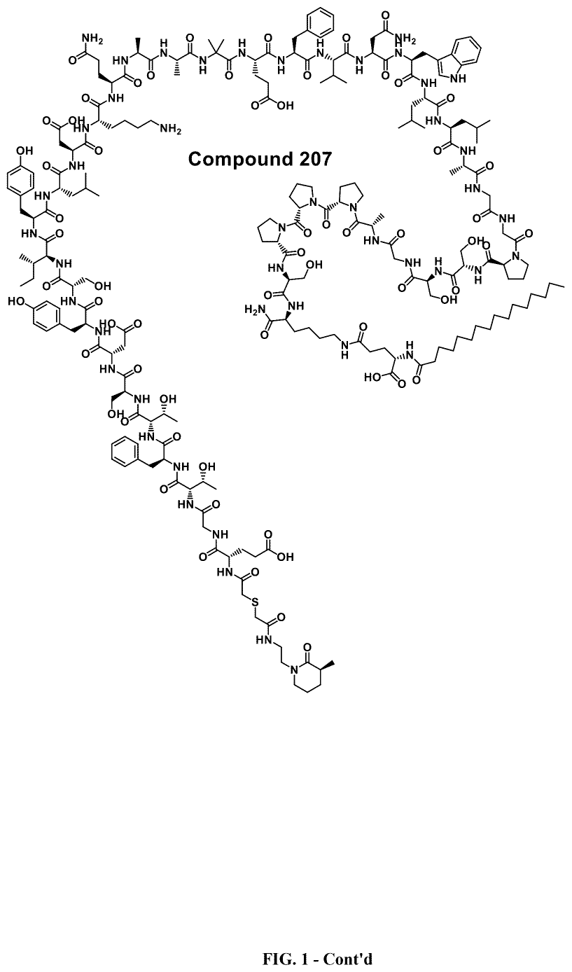

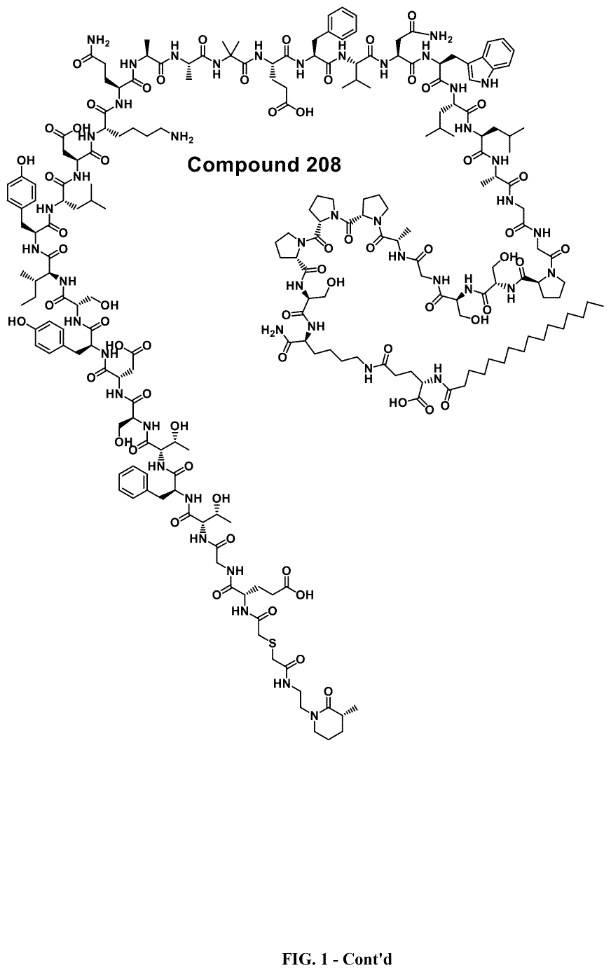

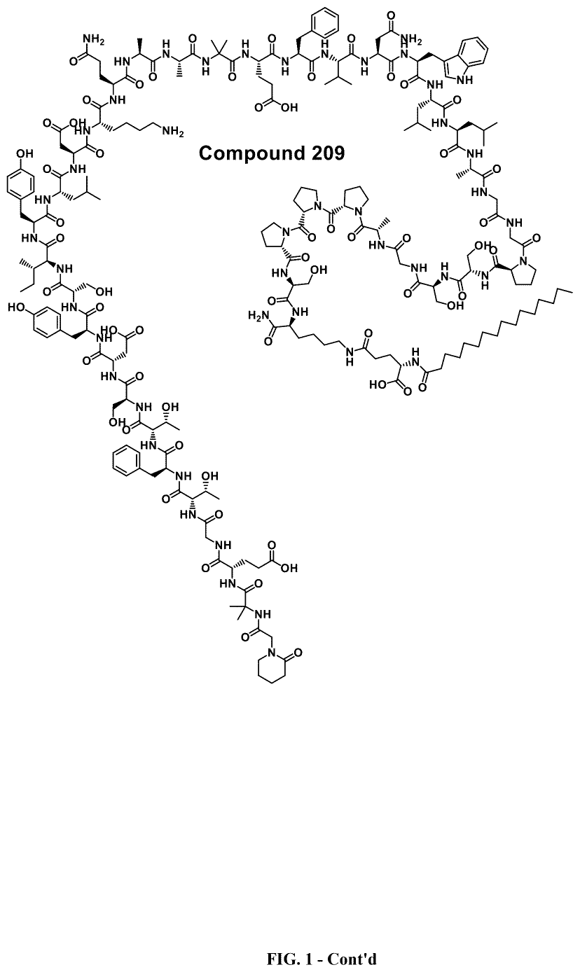

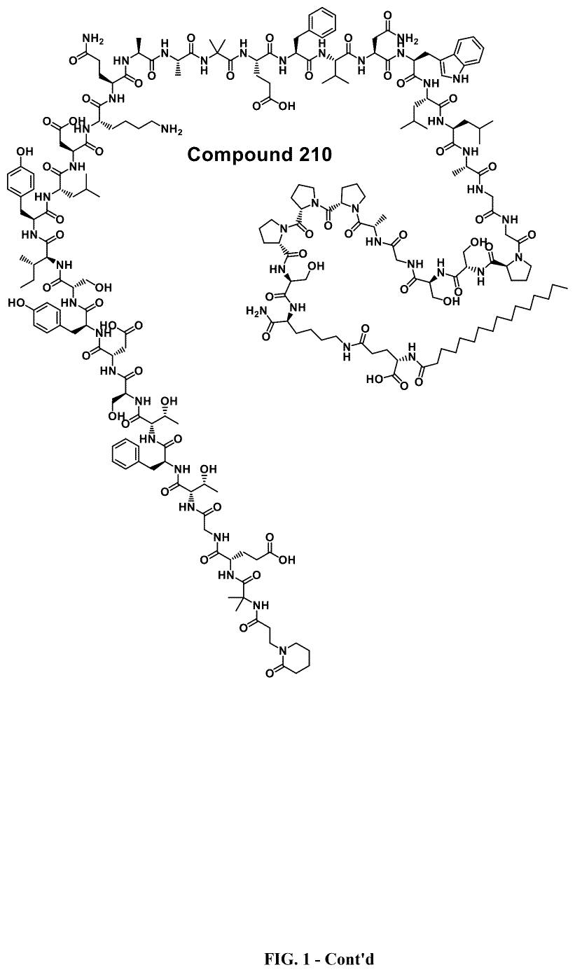

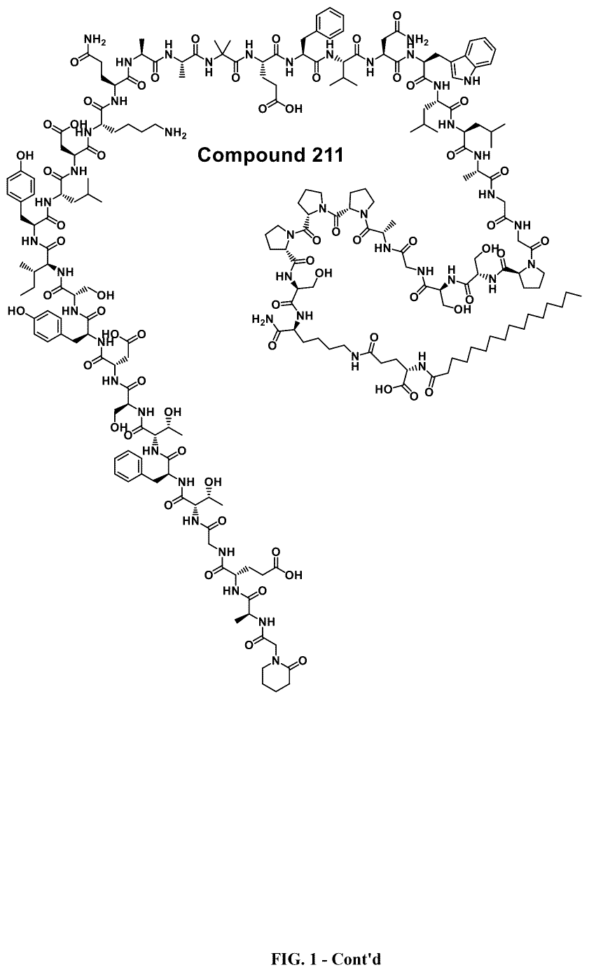

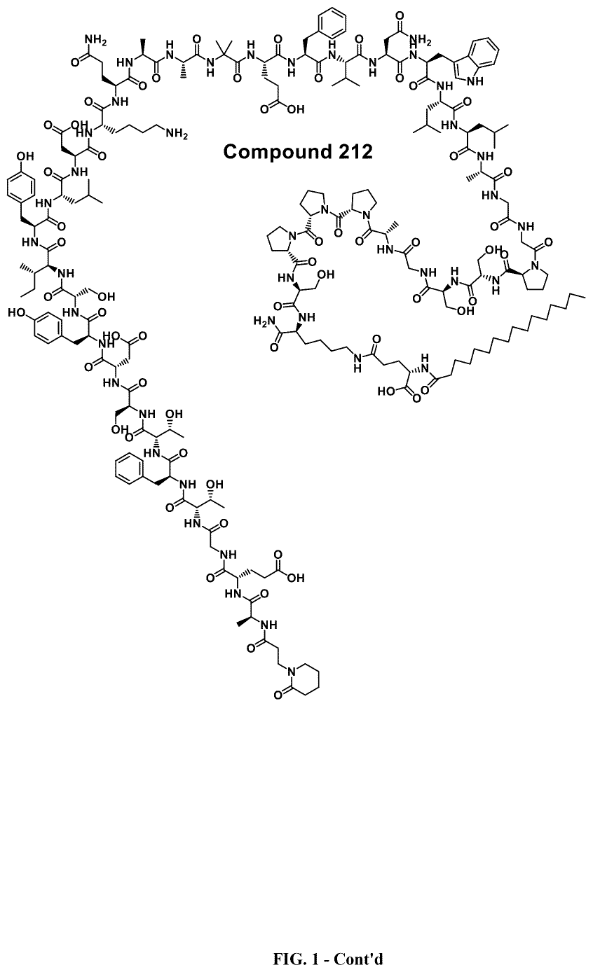

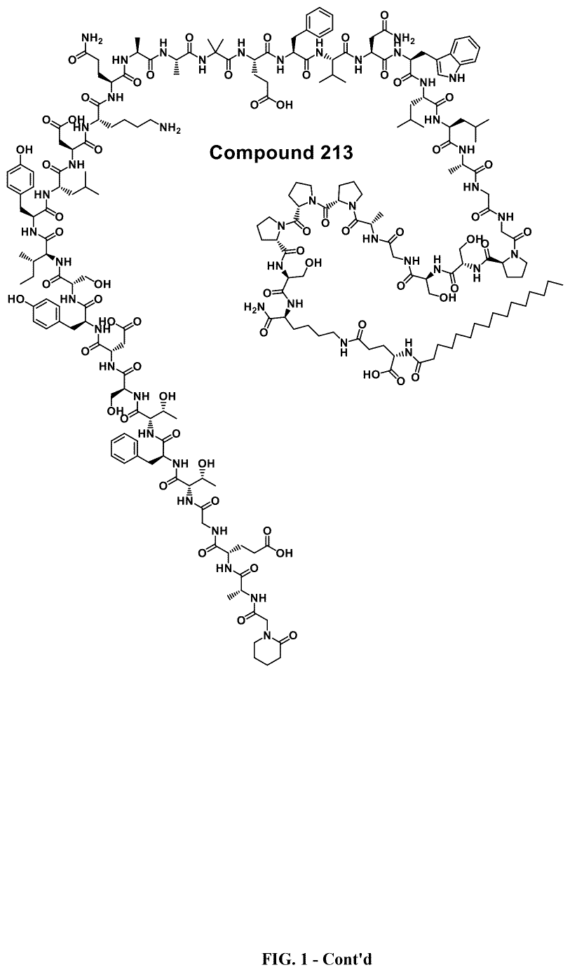

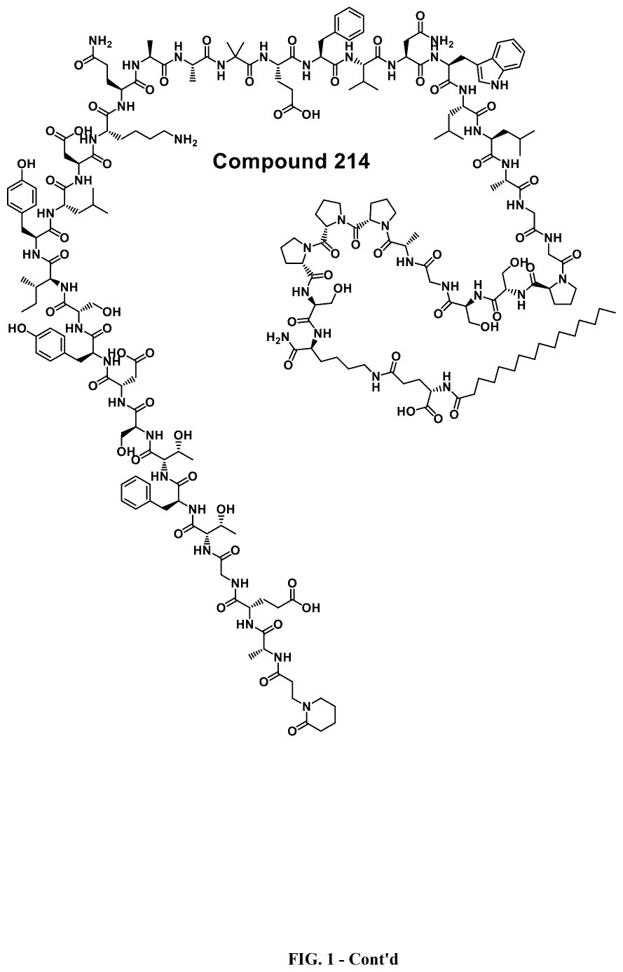

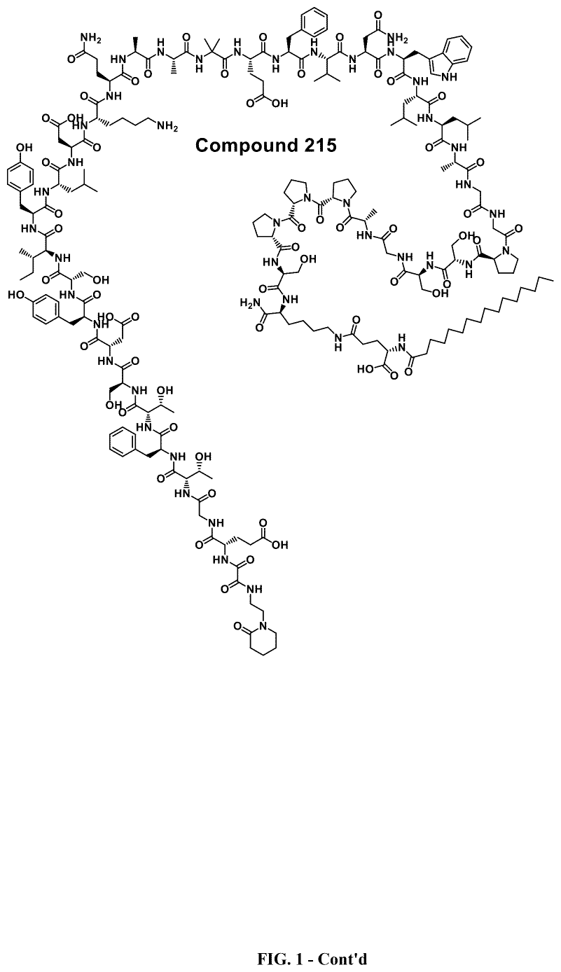

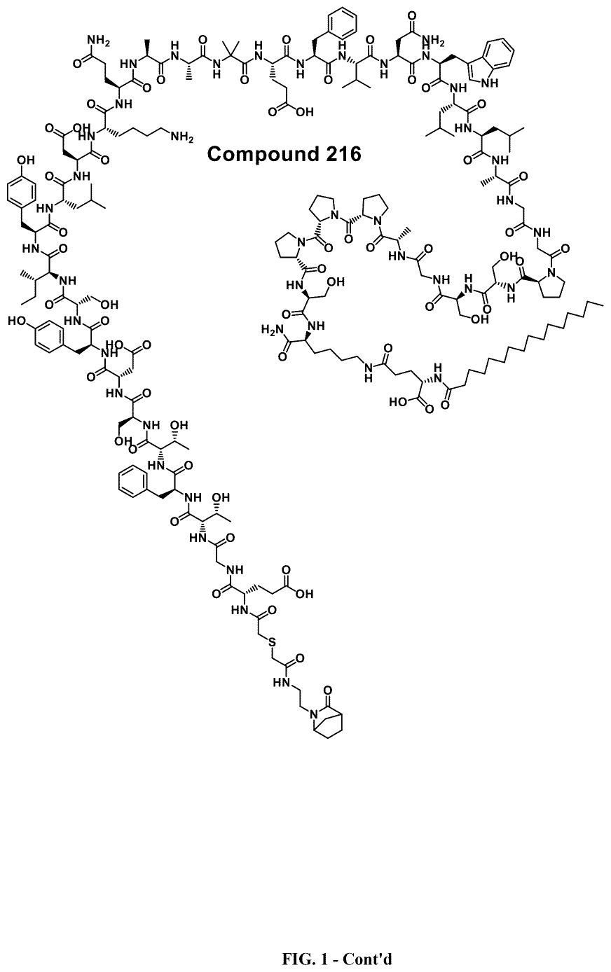

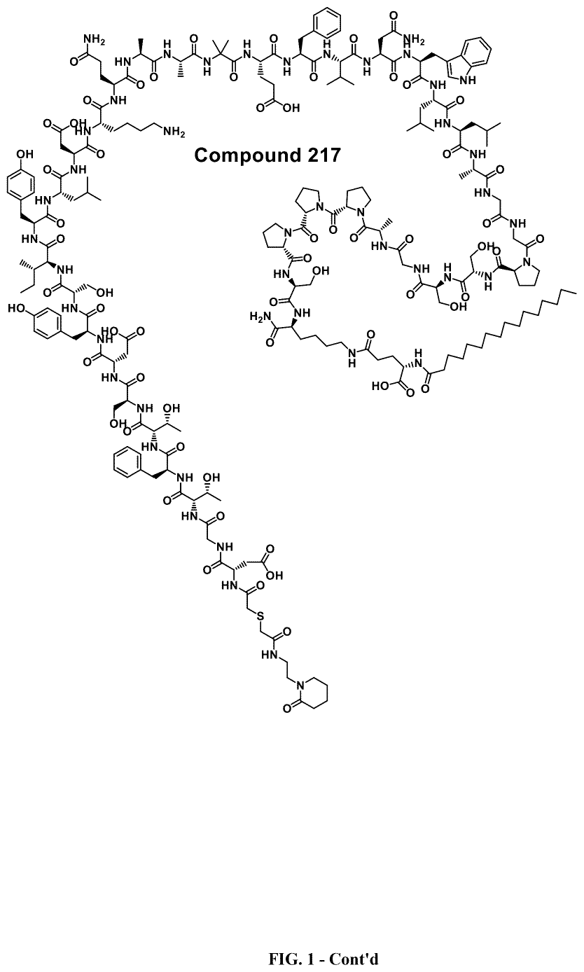

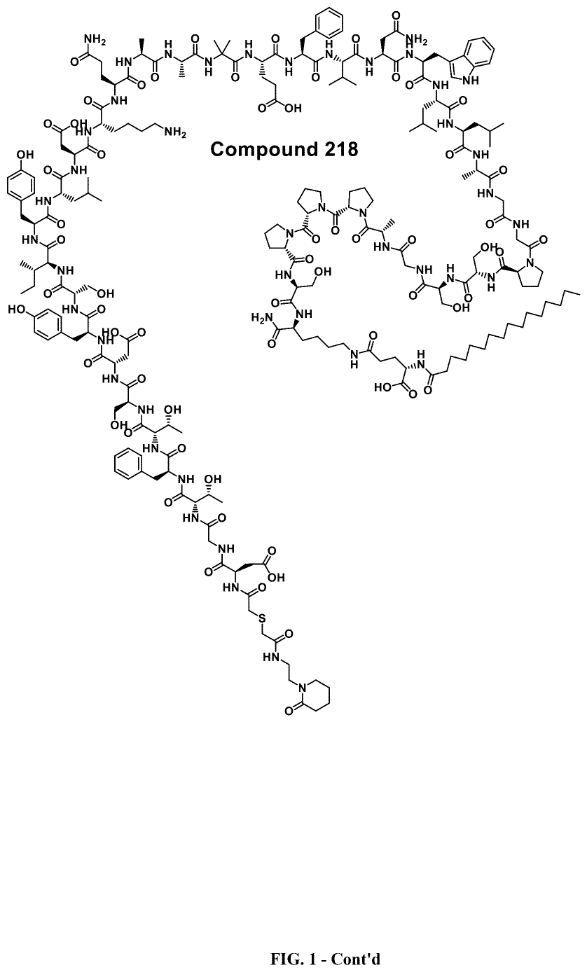

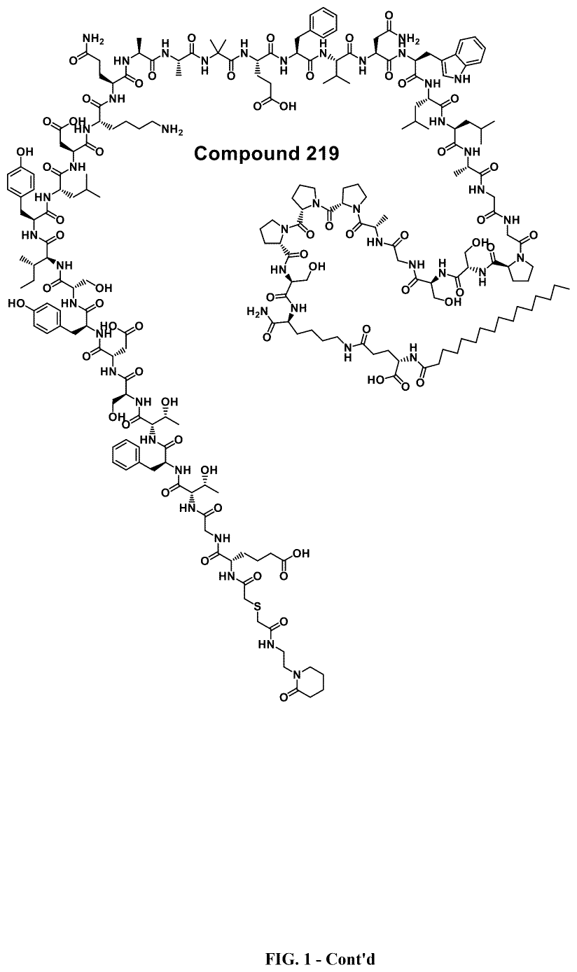

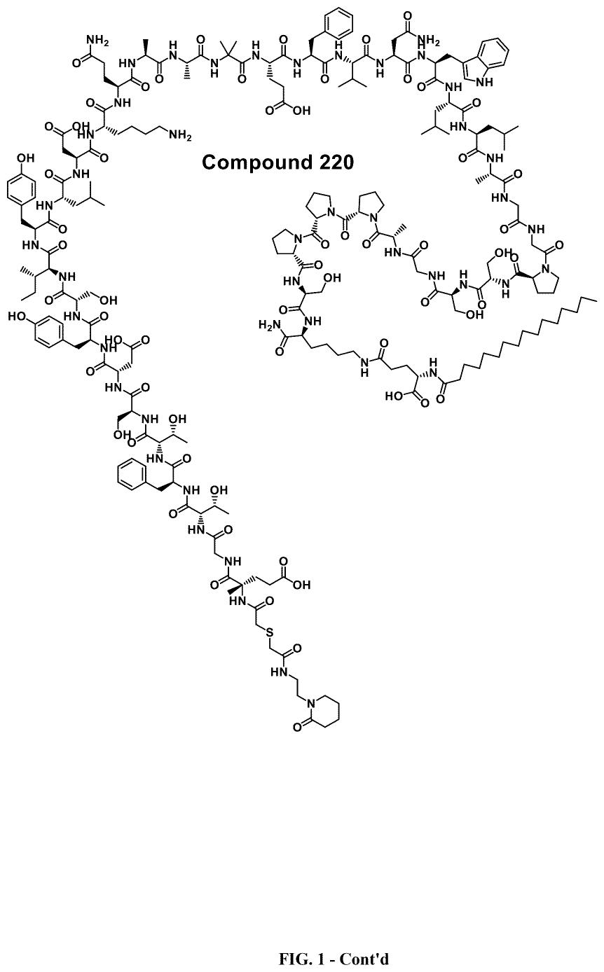

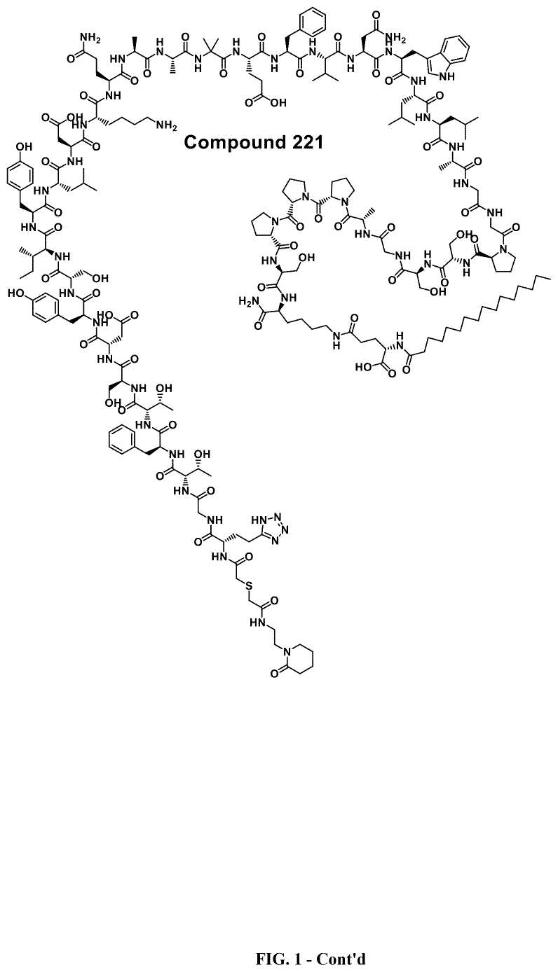

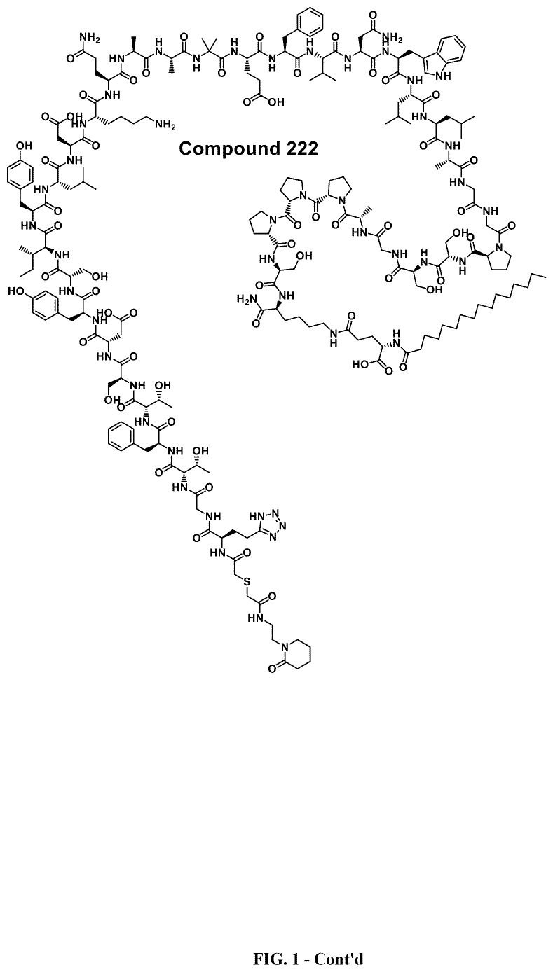

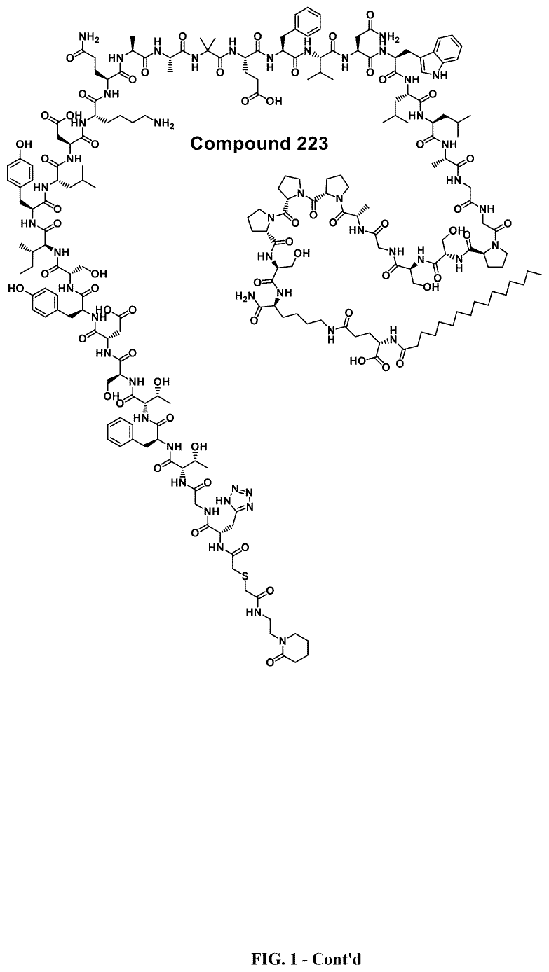

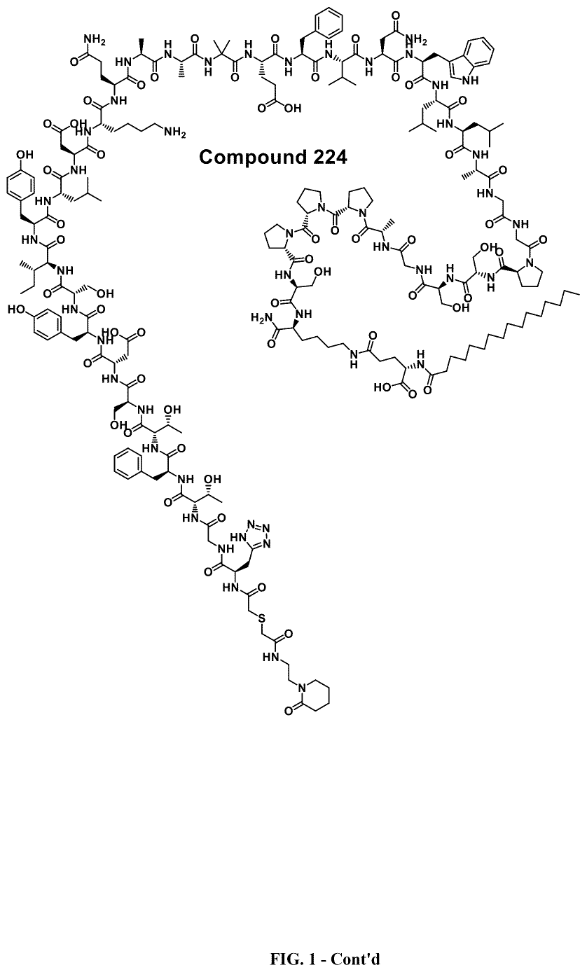

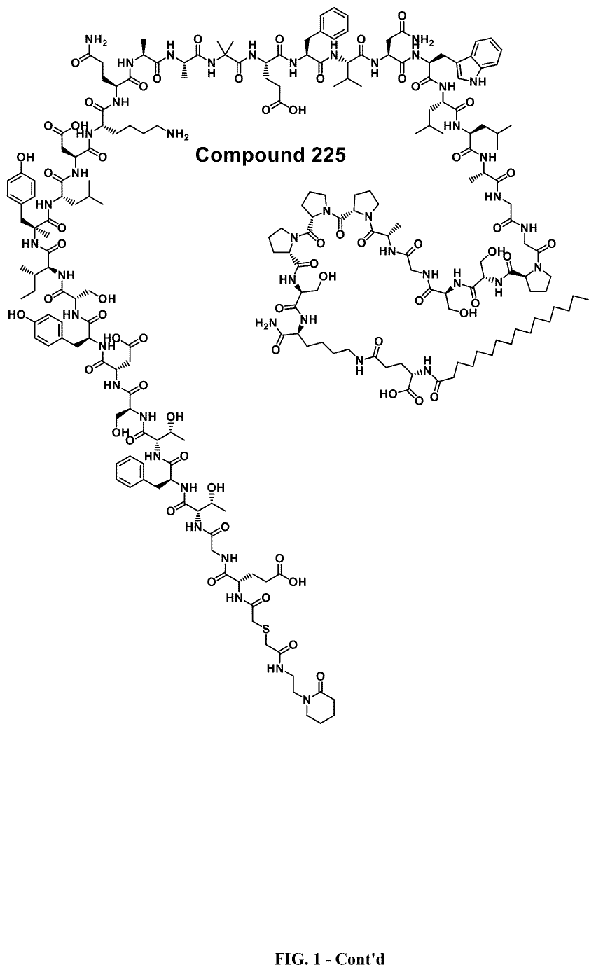

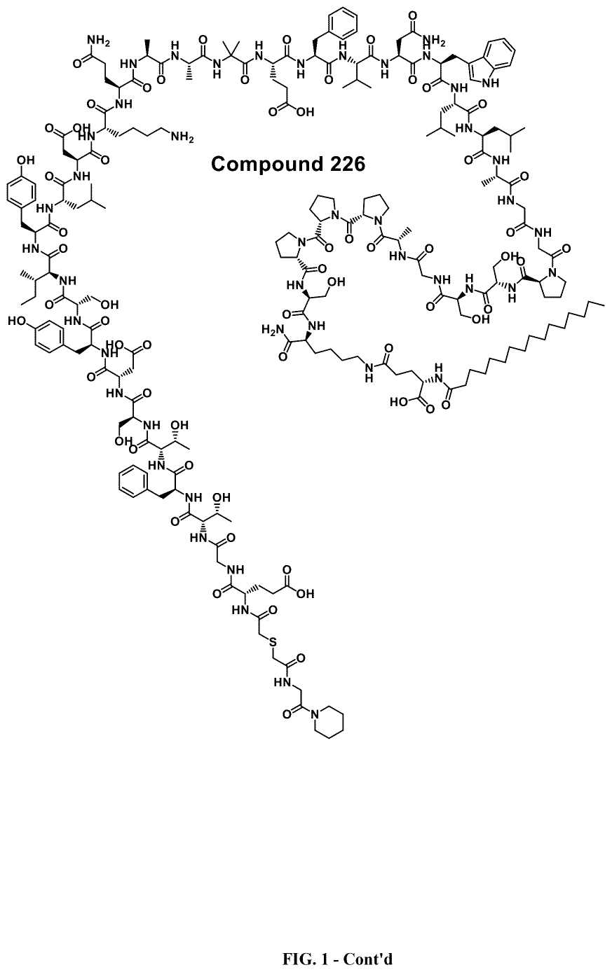

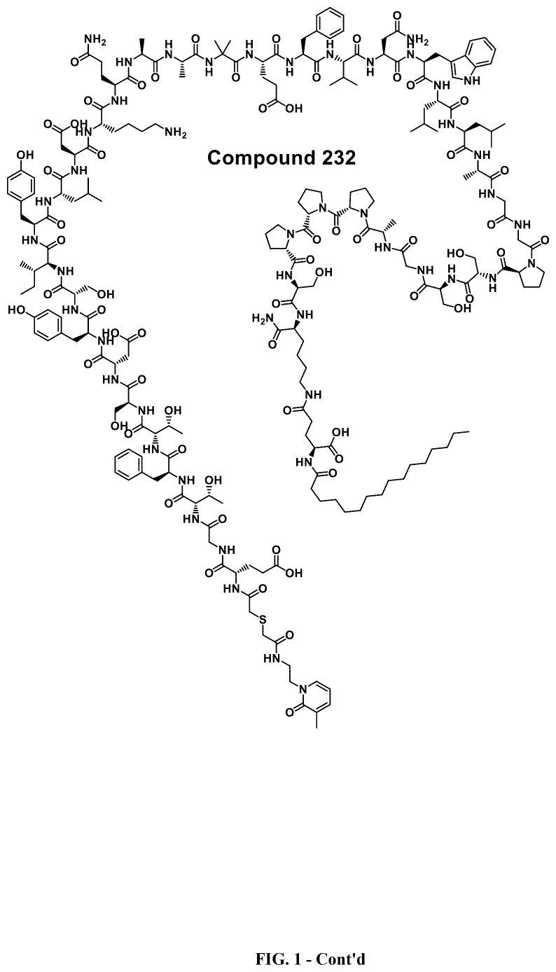

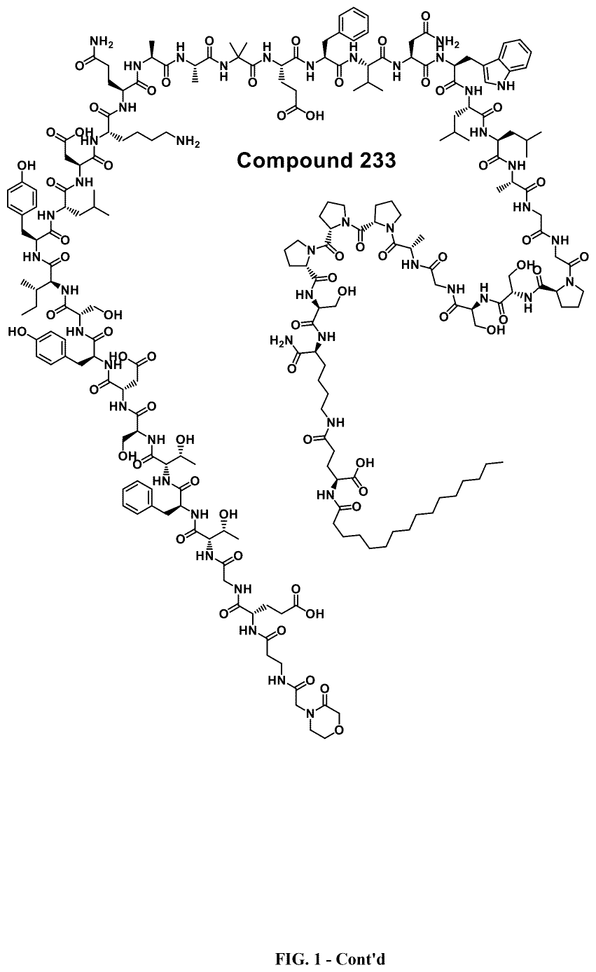

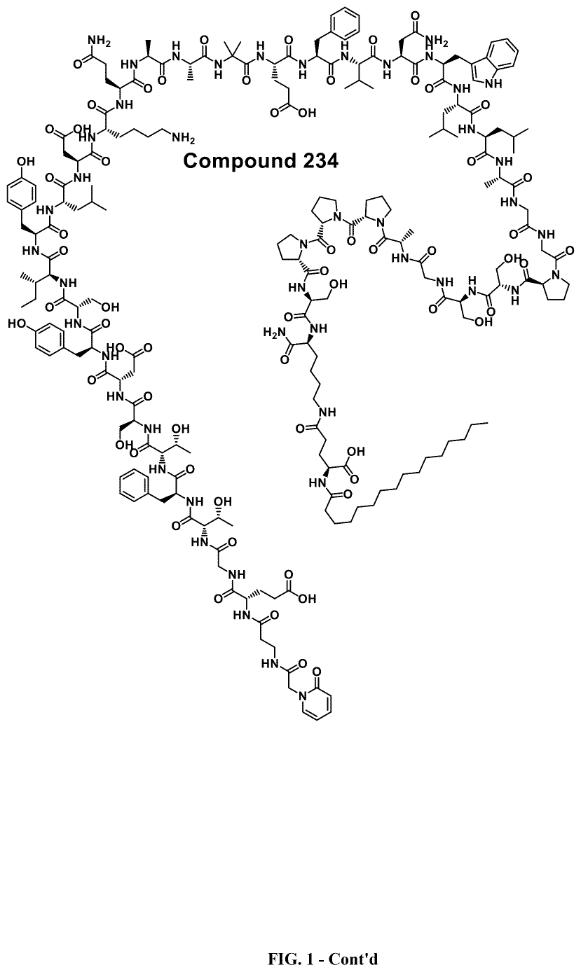

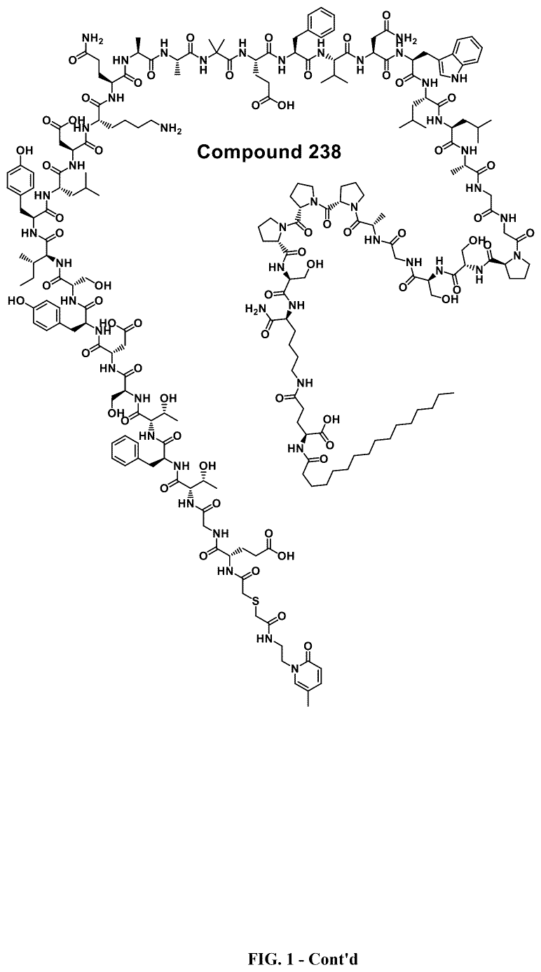

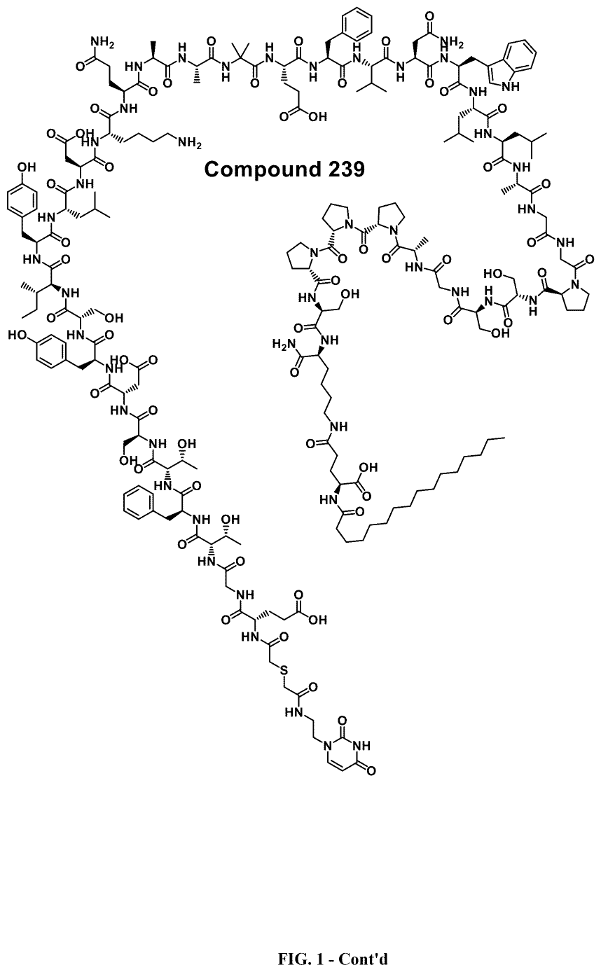

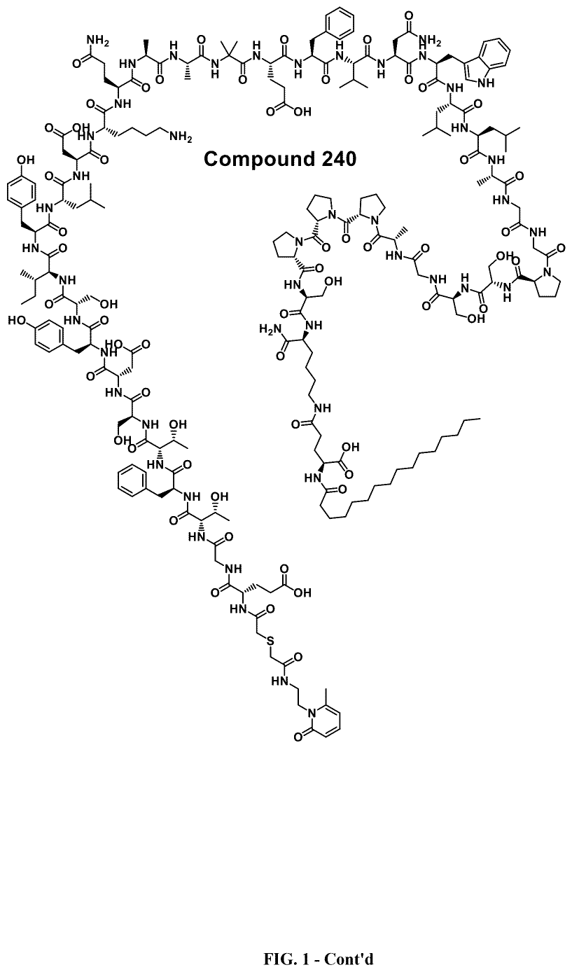

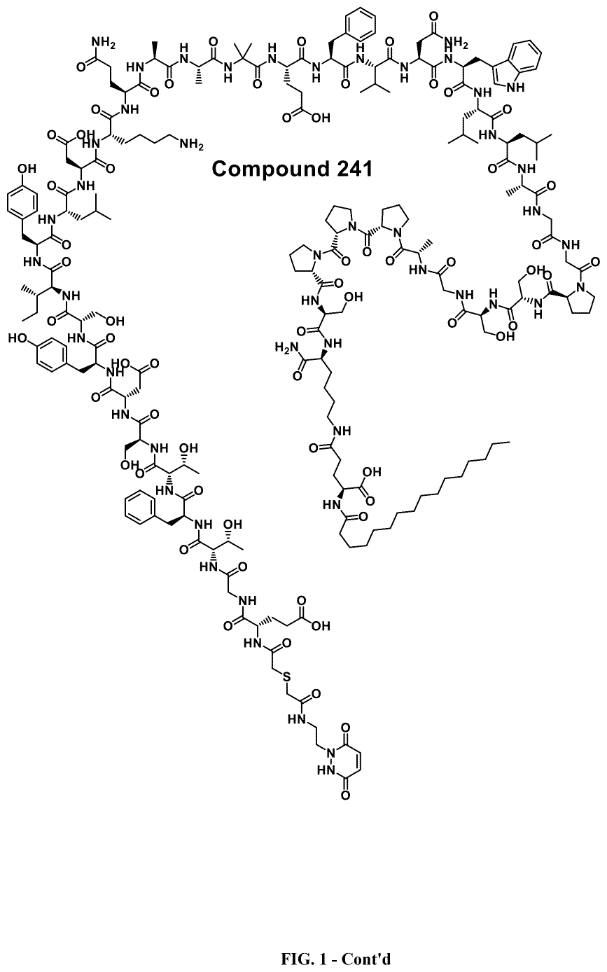

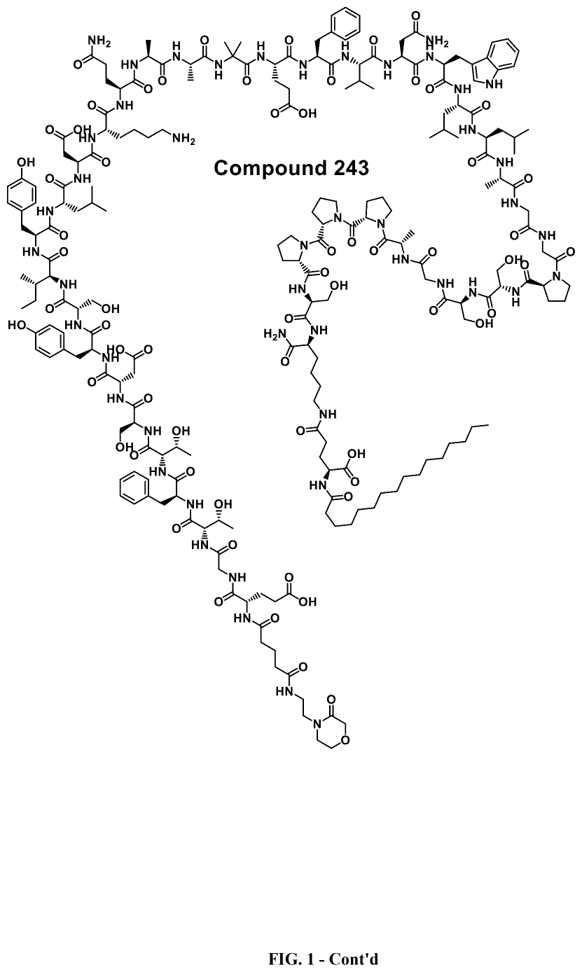

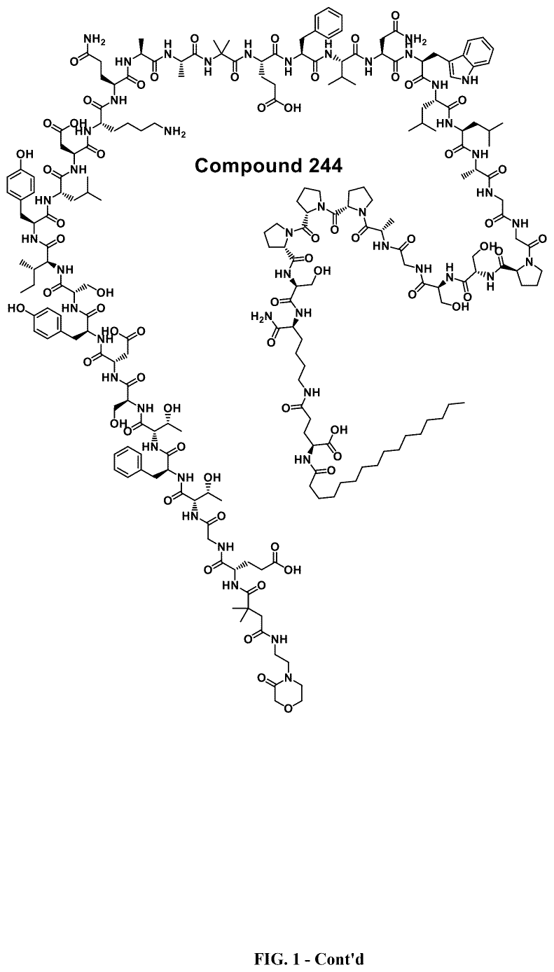

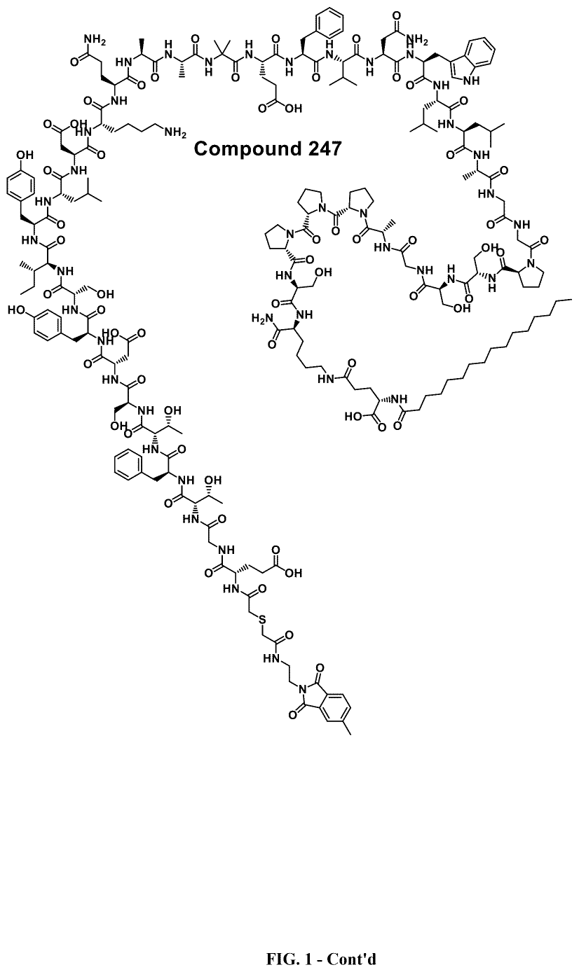

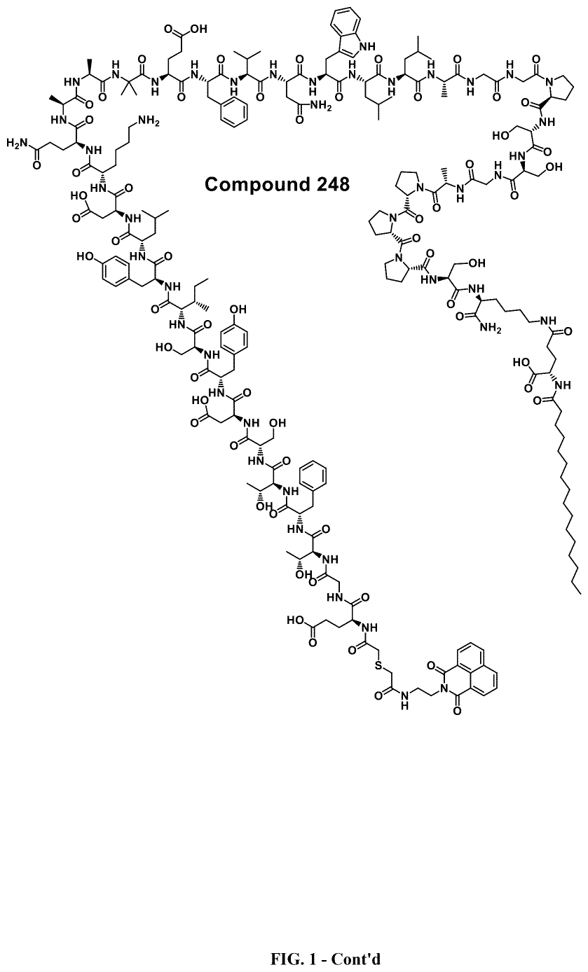

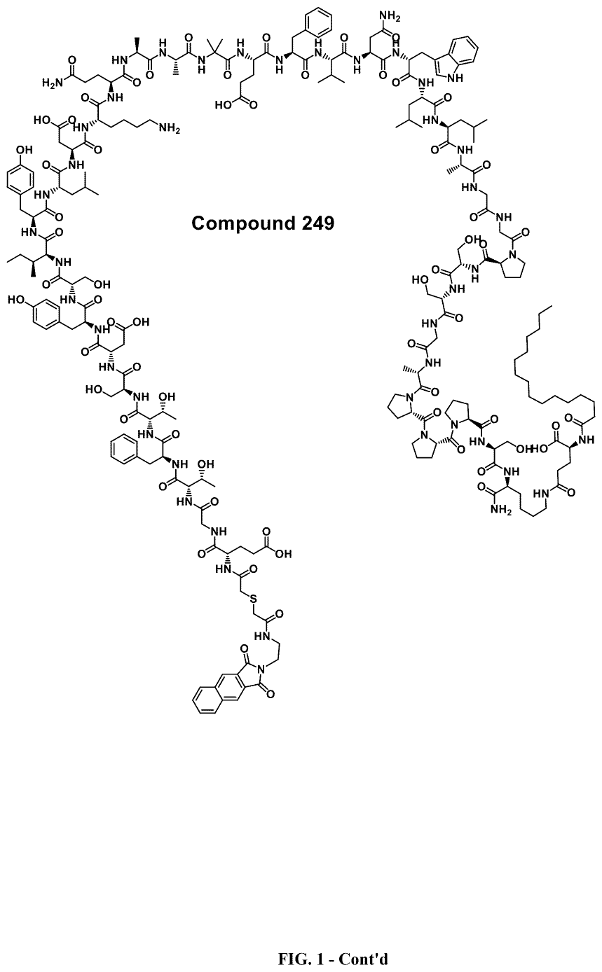

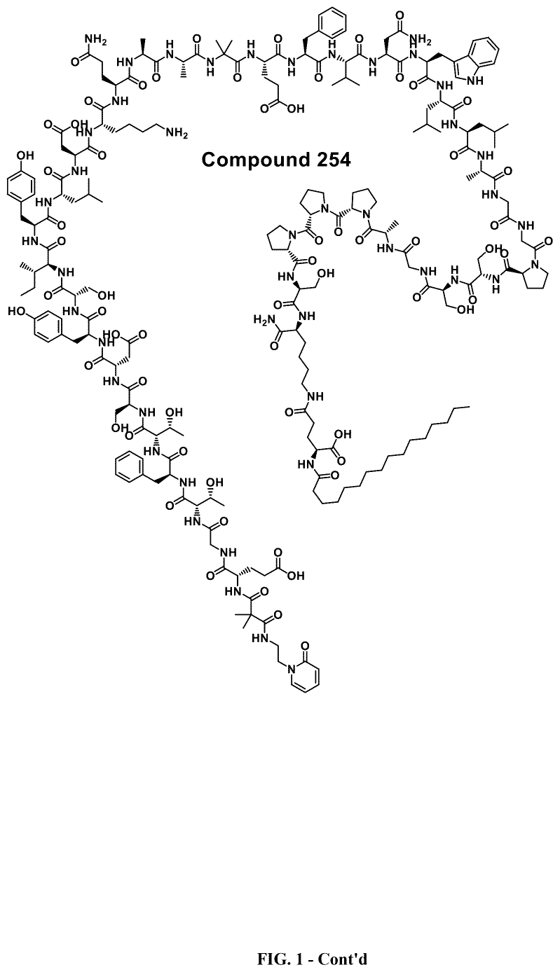

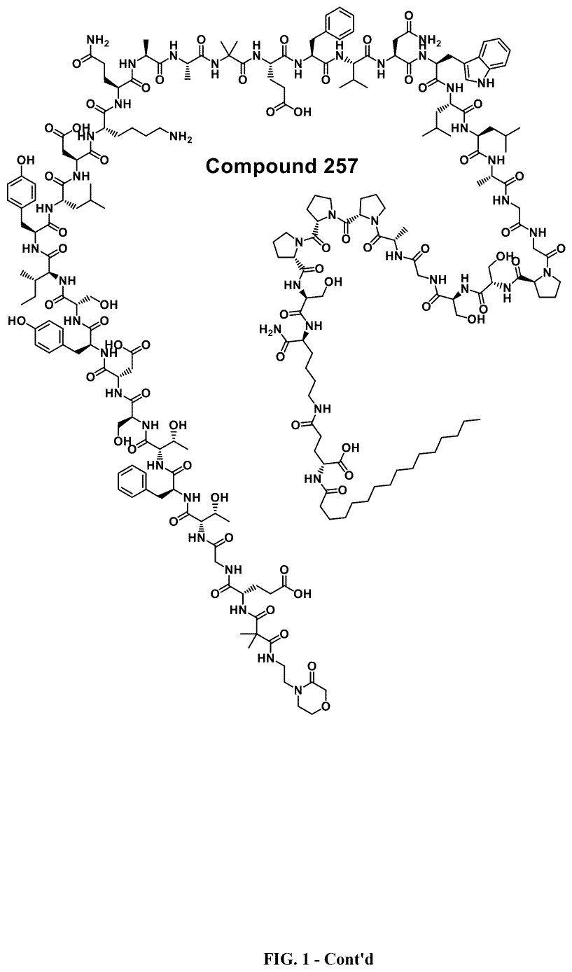

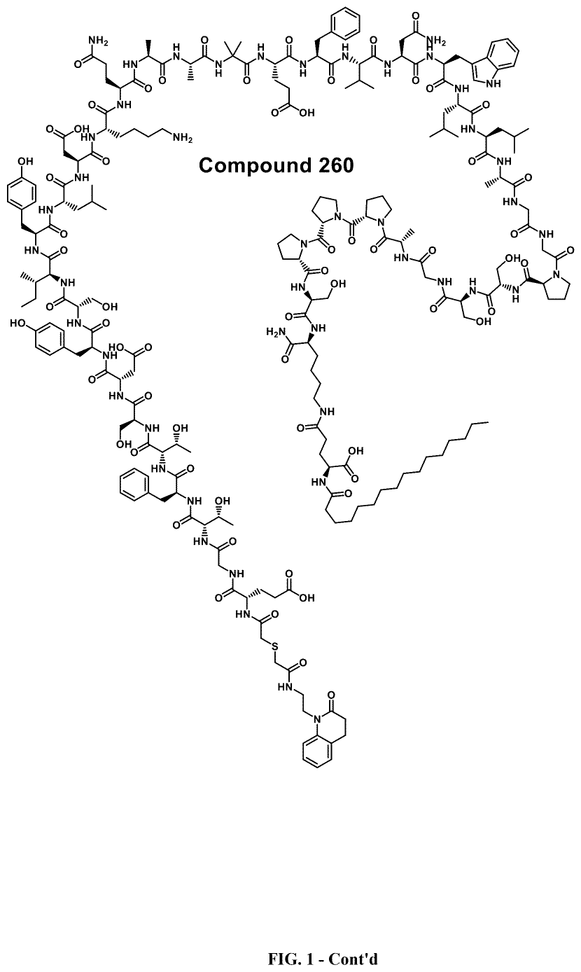

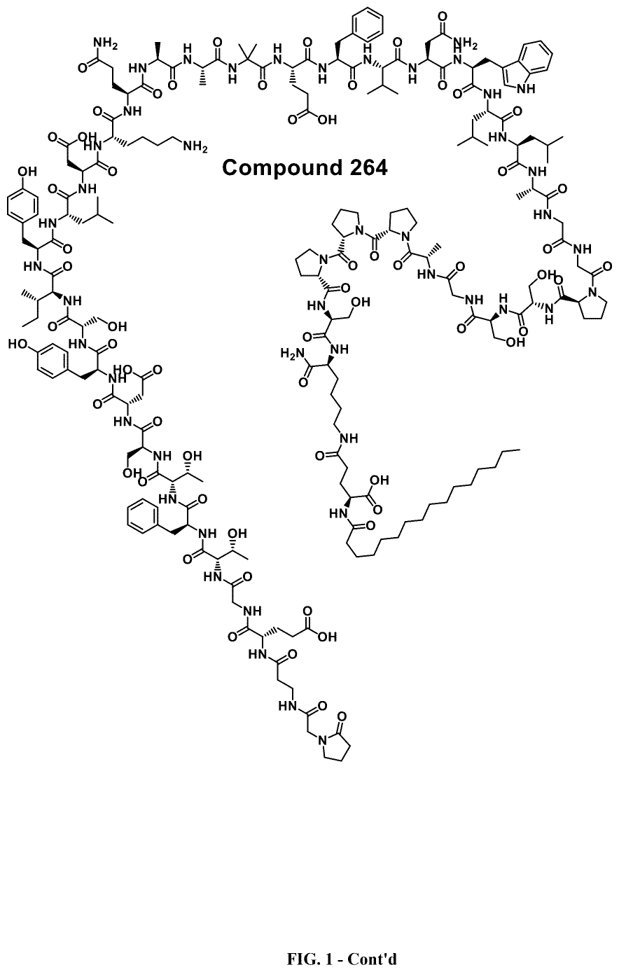

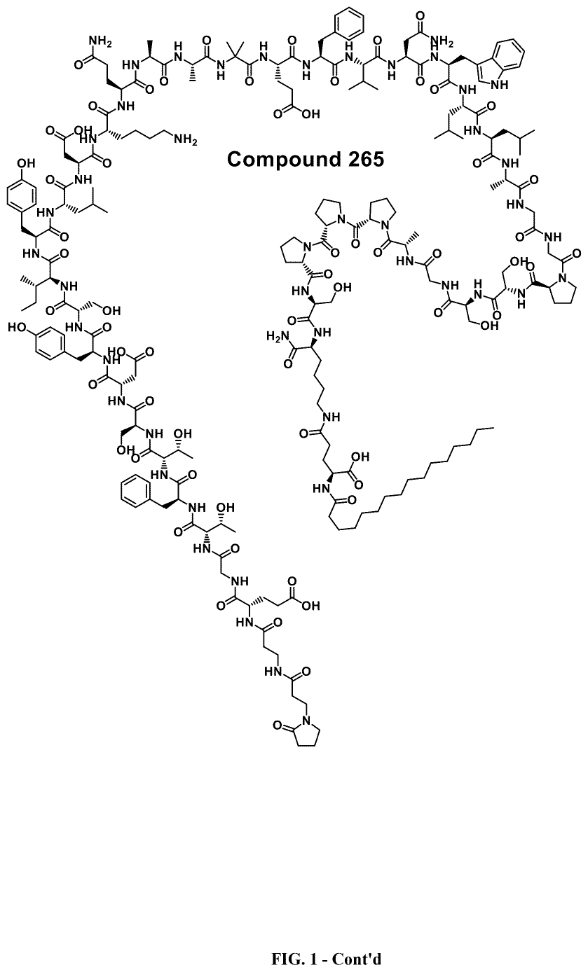

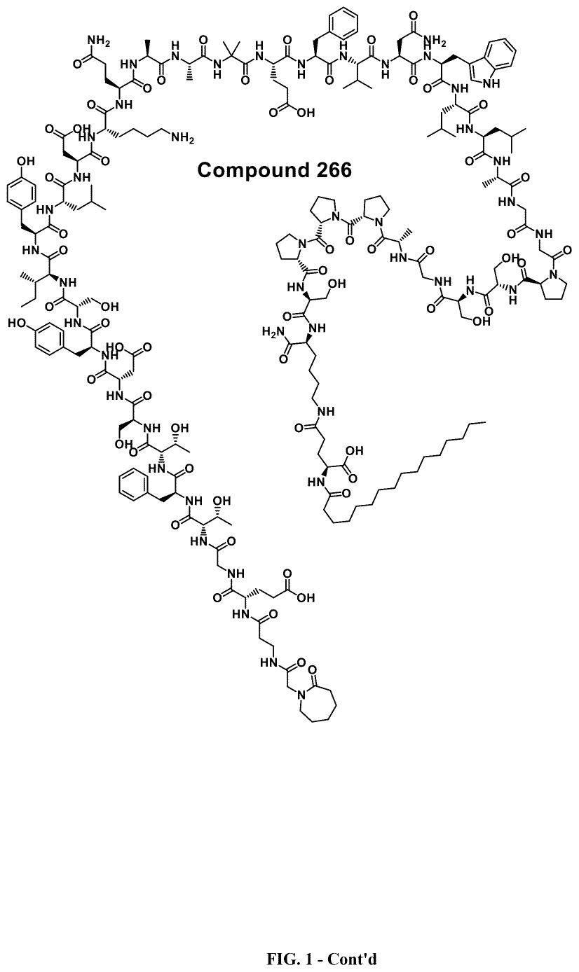

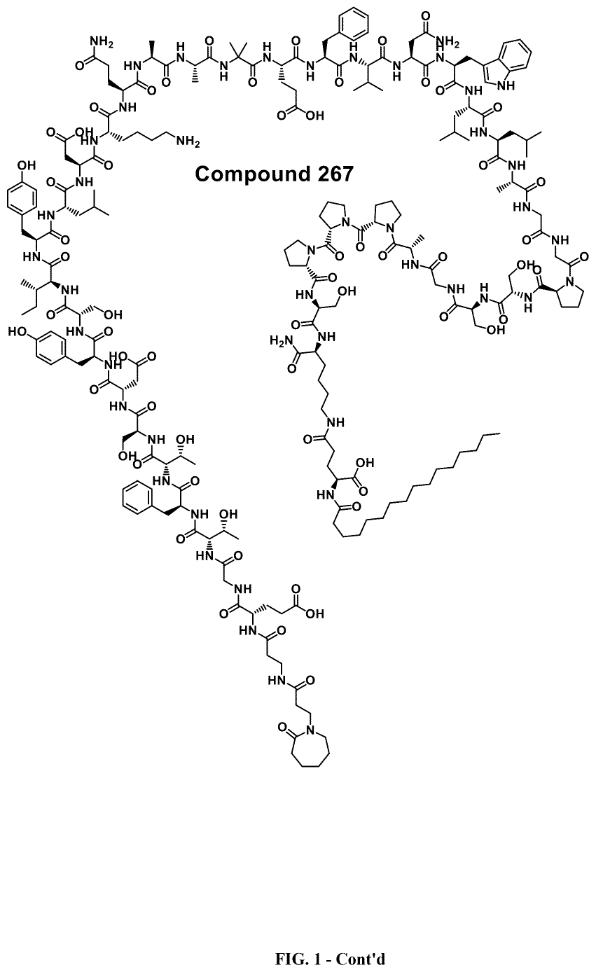

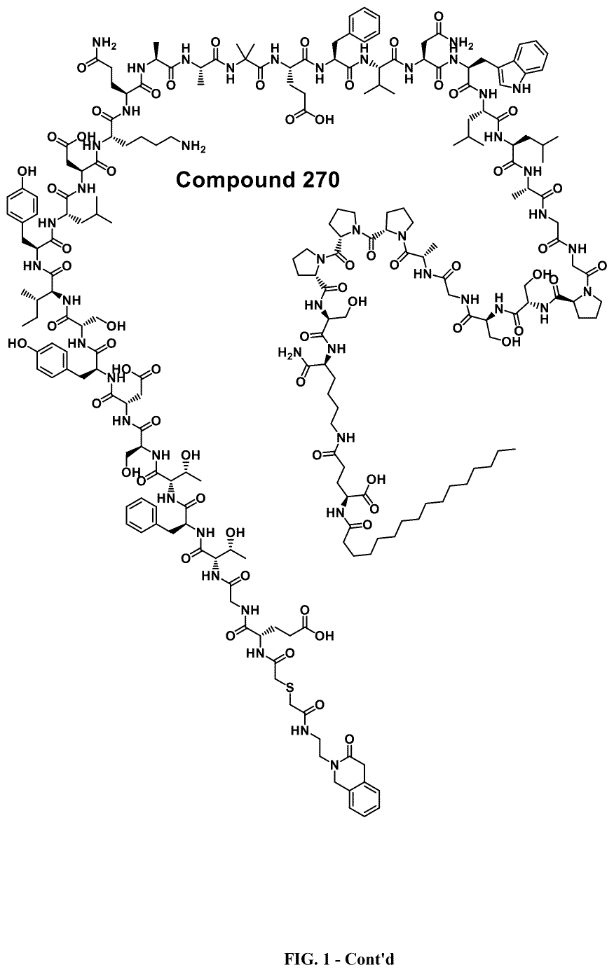

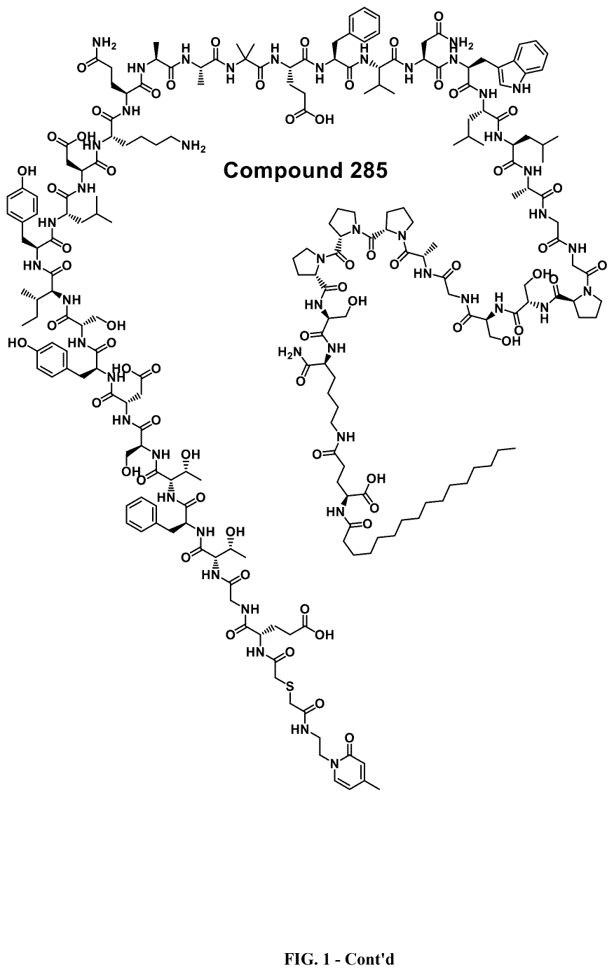

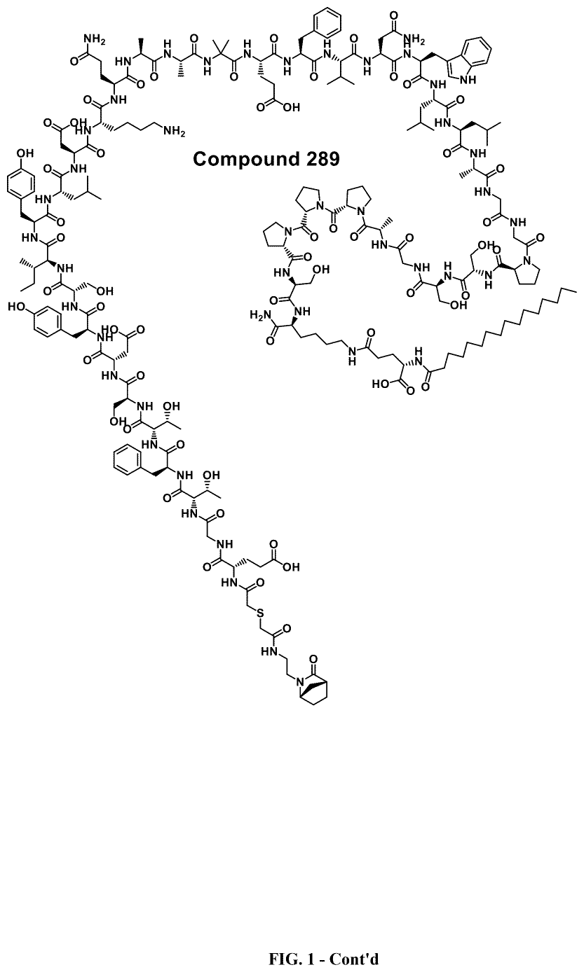

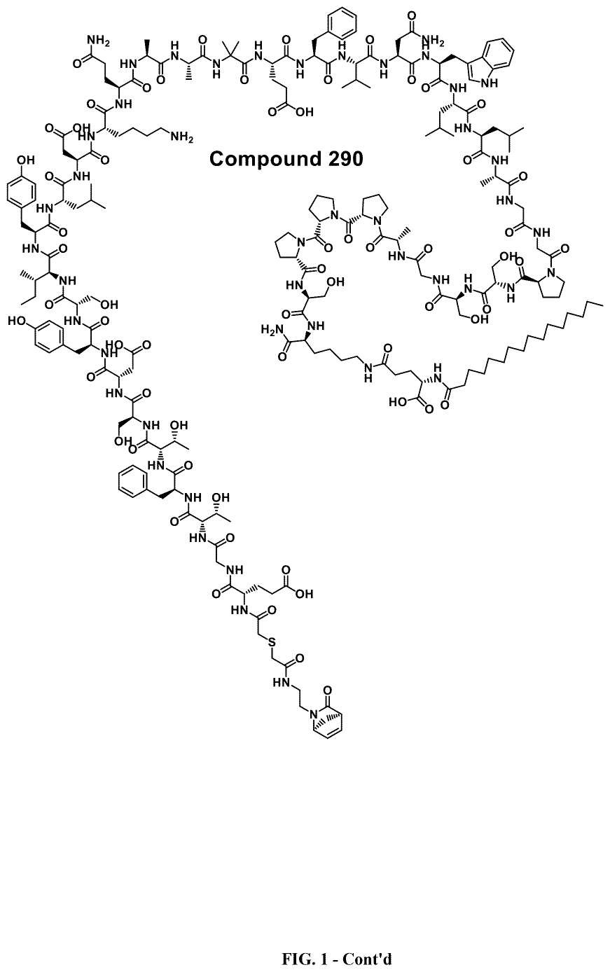

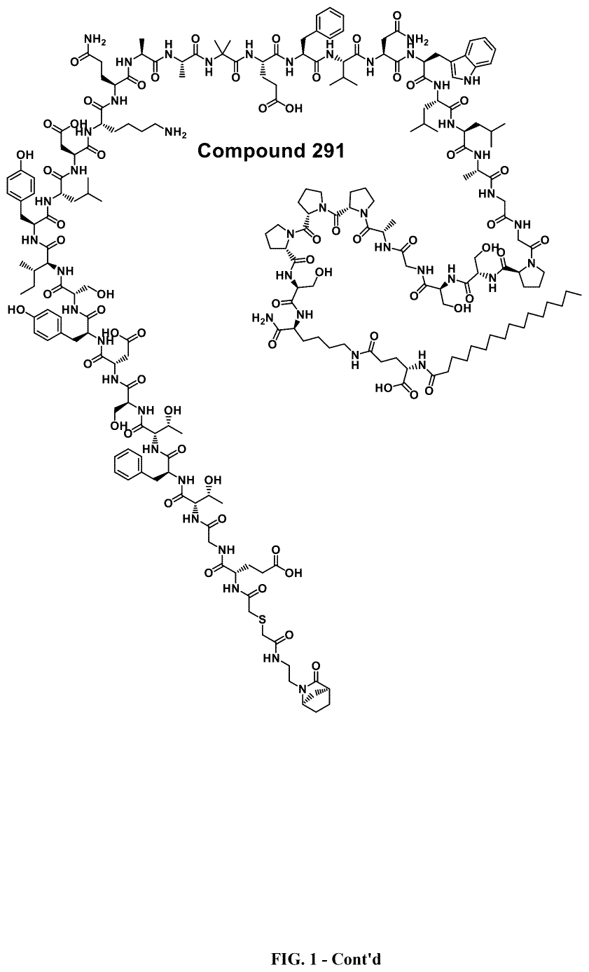

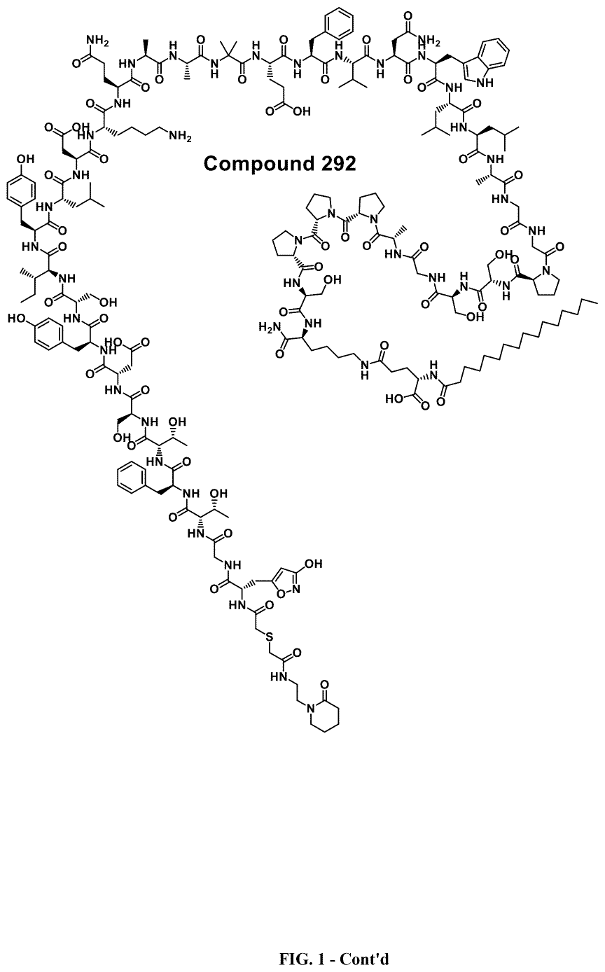

### ADDITIONAL DEFINITIONS

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “GLP-1R” is meant to include, without limitation, nucleic acids, polynucleotides, oligonucleotides, sense and antisense polynucleotide strands, complementary sequences, peptides, polypeptides, proteins, homologous and/or orthologous GLP-1R molecules, isoforms, precursors, mutants, variants, derivatives, splice variants, alleles, different species, and active fragments thereof.

As used herein, the term “GIPR” is meant to include, without limitation, nucleic acids, polynucleotides, oligonucleotides, sense and antisense polynucleotide strands, complementary sequences, peptides, polypeptides, proteins, homologous and/or orthologous GIPR molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The term “IC50” or “EC50” refers an amount, concentration, or dosage of a compound that is required for 50% inhibition or activation of a maximal response observed for such compound (or that of a reference compound as the case may be) in an assay that measures such response.

The terms “effective amount” or “therapeutically effective amount,” as used herein, refer to a sufficient amount of a chemical entity (e.g., a compound described generically or specifically herein, a pharmaceutically acceptable salt thereof, or compositions containing the same) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “haloalkoxy” refers to an —O-haloalkyl radical (e.g., —OCH₃).

The term “alkylene” refers to a branched or unbranched divalent alkyl (e.g., —CH₂—).

The term “arylene” and the like refer to divalent forms of the ring system, here divalent aryl.

The term “alkenyl” refers to a hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it.

The term “aryl” refers to a 6-carbon monocyclic, 10-carbon bicyclic, or 14-carbon tricyclic aromatic ring system wherein 0, 1, 2, 3, or 4 atoms of each ring may be substituted by a substituent, and wherein the ring comprising a monocyclic radical is aromatic and wherein at least one of the fused rings comprising a bicyclic or tricyclic radical is aromatic e.g. tetrahydronaphthyl. Examples of aryl groups also include phenyl, naphthyl and the like.

The term “cycloalkyl” as used herein includes saturated cyclic hydrocarbon groups having 3 to 10 carbons, preferably 3 to 8 carbons, and more preferably 3 to 6 carbons, wherein the cycloalkyl group may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent, and wherein the ring comprising a monocyclic radical is aromatic and wherein at least one of the fused rings comprising a bicyclic or tricyclic radical is aromatic (but does not have to be a ring which contains a heteroatom, e.g. tetrahydroisoquinolinyl. Examples of heteroaryl groups also include pyridyl, furyl or furanyl, imidazolyl, benzimidazolyl, pyrimidinyl, thiophenyl or thienyl, quinolinyl, indolyl, thiazolyl, and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The details of one or more embodiments of the invention are set forth in the description below and in the drawings. Other features and advantages will also be apparent from the claims.

### DETAILED DESCRIPTION

This disclosure features chemical entities (e.g., a compound or a pharmaceutically acceptable salt and/or hydrate and/or prodrug of the compound) that modulate (e.g., agonize or partially agonize or antagonize) glucagon-like peptide-1 receptor (“GLP-1R”) and/or the gastric inhibitory polypeptide receptor (“GIPR”). The chemical entities are useful, e.g., for treating a subject (e.g., a human) having a disease, disorder, or condition in which modulation (e.g., agonism, partial agonism or antagonism) of GLP-1R and/or GIPR activities is beneficial for the treatment or prevention of the underlying pathology and/or symptoms and/or progression of the disease, disorder, or condition. In some embodiments, the modulation results in an enhancement of (e.g., an increase in) existing levels (e.g., normal or below normal levels) of GLP-1R and/or GIPR activity (e.g., signaling). In some embodiments, the chemical entities described herein further modulate (e.g., attenuate, uncouple) β-arrestin signaling relative to what is observed with the native ligand. This disclosure also features compositions as well as other methods of using and making the said chemical entities.

In one aspect, the disclosure provides a compound having formula (IAA), or a pharmaceutically acceptable salt thereof:

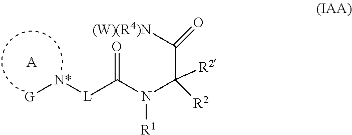

wherein:
ring A is:
(i) a saturated or unsaturated monocyclic ring that includes from 3-8 ring atoms (inclusive of G and the nitrogen atom labelled N*); or
(ii) a saturated or unsaturated bicyclic or tricyclic ring that includes from 6-14 ring atoms (inclusive of G and the nitrogen atom labelled N*), wherein:
   G is C(O), S(O), or SO₂; and
   the dotted, circular line connecting G and N* is a divalent group that includes from 1-6 ring atoms; wherein:
(a) from 0-2 of the divalent group's ring atoms are ring heteroatoms, which are each independently selected from the group consisting of N, N(R^{a}), O, S, and SO₂; and
(b) from 1-6 of the divalent group's ring atoms are ring carbon atoms, which are each independently selected from the group consisting of C, CH, CH₂, CR^{b}, C(R^{b})₂, and CHR^{b};
wherein:
(1) when ring A is a monocyclic ring, then each of the divalent group's 1-6 ring carbon atoms is independently selected from the group consisting of CH, CH₂, CR^{b}, C(R^{b})₂, and CHR^{b}; and
(2) when ring A is a bicyclic or tricyclic ring, then (A) or (B) applies:
(A) two or three adjacent ring carbon atoms of the divalent group are each independently selected from the group consisting of the group consisting of C, CH, CH₂, CR^{b}, C(R^{b})₂, and CHR^{b} and are fused to a second ring that is selected from the group consisting of:
(a) C₆₋₁₀ aryl optionally substituted with from 1-5 independently selected R^{c};
(b) heteroaryl including from 5-10 ring atoms, wherein from 1-4 ring atoms are each independently selected from the group consisting of N, N(R^{a}), O, and S, wherein the heteroaryl is optionally substituted with from 1-3 independently selected Re;
(c) C₃₋₁₀ cycloalkyl optionally substituted with from 1-4 independently selected Rd;
(d) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected Rd; or
(B) one of the divalent group's ring carbon atoms is C and is spiro-fused to a second ring that is selected from the group consisting of:
(a) C₃₋₁₀ cycloalkyl optionally substituted with from 1-4 independently selected Rd;
(b) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected Rd;
L is:
(i) —(CH₂)ₘ—X¹—(CH₂)ₙ—X²—(CH₂)ₚ— (formula VIII), wherein mis from 1-6; n is from 0-2; and p is from 0-8;
(ii) —C(O)—(CH₂)ₙ—X³—(CH₂)ₚ— (formula IX), wherein nis from 0-2; and p is from 0-8;
(iii) —(CH₂)_{q}—, wherein q is from 1-10;
(iv) —C(O)—; or
(v) —(CH₂)ₘ—X¹—(CH₂)ₚ— (formula VIII), wherein m is from 1-6; and p is from 0-8;
X¹ is —C(O)—; —N(R′)C(O)—; —C(O)N(R′)—; or —N(R′)C(O)NR′)—; wherein each occurrence of R′ is independently selected from the group consisting of: H and C₁₋₄ alkyl;
X² is:
(i) —O—;
(ii) —S—;
(iii) —S(O)ₜ—, wherein t is 1 or 2;
(iv) —C(Q¹)(Q²)-, wherein each of Q¹ and Q² is independently selected from the group consisting of H and C₁₋₄ alkyl; or Q¹ and Q², together with the carbon atom to which each is attached forms C₃₋₁₀ cycloalkyl that is optionally substituted with from 1-4 independently selected Rd and optionally fused to phenyl;
(v) C₆₋₁₀ arylene optionally substituted with from 1-5 independently selected R^{c};
(vi) heteroarylene including from 5-10 ring atoms, wherein from 1-4 ring atoms are each independently selected from the group consisting of N, N(R^{a}), O, and S; and which is optionally substituted with from 1-5 independently selected R^{c};
(vii) C₃₋₁₀ cycloalkylene optionally substituted with from 1-4 independently selected R^{d};
(viii) heterocycloalkylene including from 3-10 ring atoms, wherein from 1-3 ring atoms are each independently selected from the group consisting of N(R^{a}), O, and S; and which is optionally substituted with from 1-5 independently selected R^{d};
(ix) C₂-C₄ alkenylene optionally substituted with from 1-2 R^{e}; or
(x) C₂-C₄ alkynylene optionally substituted with from 1-2 R^{e};
X³ is: —O—; —S—; or —S(O)ₜ—, wherein tis 1 or 2;
each of R¹, R², and R⁴ is independently selected from the group consisting of H and unsubstituted C₁₋₃ alkyl;
R^{2′} is as defined according to (AA) or (BB) below:

### (AA)

R^{2′} is

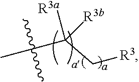

wherein:
R³ is —C(O)OH, —C(O)OR³¹, —CH(C(O)OH)₂ or a carboxylic acid isostere (e.g., amide or tetrazolyl);
a is 0-5;
a′ is 0 or 1; and
each of R^{3a} and R^{3b} is independently H or C₁₋₃ alkyl;

### (BB)

R^{2′} and R⁴ taken together with the atoms connecting them form a ring including from 5-8 ring atoms, wherein from 3-7 are ring carbon atoms each substituted with from 1-2 substituents independently selected from H, halo, hydroxy, oxo, and C₁₋₃ alkyl; and from 0-1 is a heteroatom (in addition to the N attached to R¹) selected from O, —NH, —N(C₁-3 alkyl), and S (e.g., R⁴ and R³, taken together with the atoms to which each is attached form a ring including 5-8 ring atoms);
R³¹ is:
(i) C₁₋₆ alkyl optionally substituted with from 1-2 independently selected Re;
(ii) —(C₀₋₃ alkylene)-C₃₋₁₀ cycloalkyl, wherein the cycloalkyl is optionally substituted with from 1-4 independently selected Rd;
(iii) —(C₀₋₃ alkylene)-heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d};
(iv) —(C₀₋₃ alkylene)-C₆₋₁₀ aryl, wherein the aryl is optionally substituted with from 1-5 independently selected Re; or
(v) —(C₀₋₃ alkylene)-heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
each occurrence of R^{a} is independently selected from the group consisting of: H; C₁₋₄ alkyl; C₃₋₆ cycloalkyl; —C(═O)(C₁₋₆ alkyl); —C(═O)O(C₁₋₆ alkyl); and —S(O)₁₋₂(C₁₋₆ alkyl);
each occurrence of R^{b} is independently selected from the group consisting of: R³¹; C₁₋₄ haloalkyl; —OH; oxo; —F; —N(R^{a})(R″); C₁₋₄ alkoxy; C₁₋₄ haloalkoxy; —C(═O)(C₁₋₄ alkyl); —C(═O)O(C₁₋₆ alkyl); —C(═O)OH, —C(═O)N(R′″)(R″′), —S(O)₁₋₂(C₁₋₆ alkyl); and cyano;
each occurrence of R^{c} is independently selected from the group consisting of: C₁₋₆ alkyl optionally substituted with from 1-2 independently selected R^{e}; C₃₋₆ cycloalkyl; C₁₋₄ haloalkyl; —OH; -halo; —NO₂; N₃; —N(R^{a})(R″); C₁₋₄ alkoxy; C₁₋₄ thioalkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{d} is independently selected from the group consisting of: C₁₋₆ alkyl optionally substituted with from 1-2 independently selected R^{e}; C₁₋₄ haloalkyl; —OH; —F; Cl; —N(R^{a})(R″); oxo; C₁₋₄ alkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{e} is independently selected from the group consisting of: —OH; —N(R^{a})(R″); C₁₋₄ alkoxy; C₁₋₄ haloalkoxy; —OC(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R″, R′″, and R″″ is independently selected from the group consisting of: H and C₁₋₆ alkyl; and
W is a peptide having the formula W′—R⁵, wherein W′ is a sequence of from 5-60 amino acids, and R⁵ is a C-terminal amino acid, amino acid ester, or amino acid amide that is optionally substituted with from 1-2 modifying groups (e.g., 1-2 groups selected from an acyl group and a PEG group).

In some embodiments, the compound has Formula (IIAA):

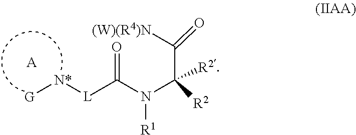

In some embodiments, the compound has Formula (IIAA-B):

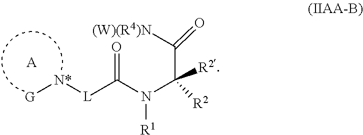

In some embodiments, the compound has Formula (IA):

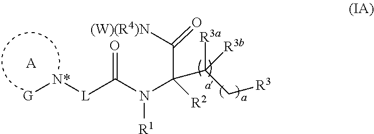

wherein:
ring A is:
(i) a saturated or unsaturated monocyclic ring that includes from 3-8 ring atoms (inclusive of G and the nitrogen atom labelled N*); or
(ii) a saturated or unsaturated bicyclic or tricyclic ring that includes from 6-14 ring atoms (inclusive of G and the nitrogen atom labelled N*), wherein:
   G is C(O), S(O), or SO₂; and
   the dotted, circular line connecting G and N* is a divalent group that includes from 1-6 ring atoms; wherein:
(a) from 0-2 of the divalent group's ring atoms are ring heteroatoms, which are each independently selected from the group consisting of N, N(R^{a}), O, S, and SO₂; and
(b) from 1-6 of the divalent group's ring atoms are ring carbon atoms, which are each independently selected from the group consisting of C, CH, CH₂, CR^{b}, C(R^{b})₂, and CHR^{b};
wherein:
(1) when ring A is a monocyclic ring, then each of the divalent group's 1-6 ring carbon atoms is independently selected from the group consisting of CH, CH₂, CR^{b}, C(R^{b})₂, and CHR^{b}; and
(2) when ring A is a bicyclic or tricyclic ring, then (A) or (B) applies:
(A) two or three adjacent ring carbon atoms of the divalent group are each independently selected from the group consisting of C, CH, and, CR^{b} and are fused to a second ring that is selected from the group consisting of:
(a) C₆₋₁₀ aryl optionally substituted with from 1-5 independently selected R^{c};
(b) heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
(c) C₃₋₁₀ cycloalkyl optionally substituted with from 1-4 independently selected R^{d};
(d) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d}; or
(B) one of the divalent group's ring carbon atoms is C and is spiro-fused to a second ring that is selected from the group consisting of:
(a) C₃₋₁₀ cycloalkyl optionally substituted with from 1-4 independently selected R^{d},
(b) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d};
L is:
(i) —(CH₂)ₘ—X¹—(CH₂)ₙ—X²—(CH₂)ₚ— (formula VIII), wherein mis from 1-6; n is from 0-2; and p is from 0-8;
(ii) —C(O)—(CH₂)ₙ—X³—(CH₂)ₚ— (formula IX), wherein nis from 0-2; and p is from 0-8;
(iii) —(CH₂)_{q}—, wherein q is from 1-10;
(iv) —C(O)—; or
(v) —(CH₂)ₘ—X¹—(CH₂)ₚ— (formula VIII), wherein m is from 1-6; and p is from 0-8;
X¹ is —C(O)—; —N(R′)C(O)—; —C(O)N(R′)—; or —N(R′)C(O)NR′)—; wherein each occurrence of R′ is independently selected from the group consisting of: H and C₁₋₄ alkyl;
X² is:
(i) —O—;
(ii) —S—;
(iii) —S(O)ₜ—, wherein tis 1 or 2;
(iv) —C(Q¹)(Q²)-, wherein each of Q¹ and Q² is independently selected from the group consisting of H and C₁₋₄ alkyl; or Q¹ and Q², together with the carbon atom to which each is attached forms C₃₋₁₀ cycloalkyl that is optionally substituted with from 1-4 independently selected R^{d} and optionally fused to phenyl;
(v) C₆₋₁₀ arylene optionally substituted with from 1-5 independently selected R^{c};
(vi) heteroarylene including from 5-10 ring atoms, wherein from 1-4 ring atoms are each independently selected from the group consisting of N, N(R^{a}), O, and S; and which is optionally substituted with from 1-5 independently selected R^{e};
(vii) C₃₋₁₀ cycloalkylene optionally substituted with from 1-4 independently selected R^{d};
(viii) heterocycloalkylene including from 3-10 ring atoms, wherein from 1-3 ring atoms are each independently selected from the group consisting of N(R^{a}), O, and S; and which is optionally substituted with from 1-5 independently selected R^{d};
(ix) C₂-C₄ alkenylene optionally substituted with from 1-2 R^{e}; or
(x) C₂-C₄ alkynylene optionally substituted with from 1-2 R^{e};
X³ is: —O—; —S—; or —S(O)ₜ—, wherein t is 1 or 2;
each of R¹, R², and R⁴ is independently selected from the group consisting of H and unsubstituted C₁₋₃ alkyl;
R³ is —C(O)OH, —C(O)OR³¹, —CH(C(O)OH)₂ or a carboxylic acid isostere (e.g., amide or tetrazolyl);
a is 0-5;
a′ is 0 or 1;
each of R^{3a} and R^{3b} is independently H or C₁₋₃ alkyl;
R³¹ is:
(i) C₁₋₆ alkyl optionally substituted with from 1-2 independently selected R^{e};
(ii) —(C₀₋₃ alkylene)-C₃₋₁₀ cycloalkyl, wherein the cycloalkyl is optionally substituted with from 1-4 independently selected R^{d};
(iii) —(C₀₋₃ alkylene)-heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d};
(iv) —(C₀₋₃ alkylene)-C₆₋₁₀ aryl, wherein the aryl is optionally substituted with from 1-5 independently selected R^{e}; or
(v) —(C₀₋₃ alkylene)-heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
each occurrence of R^{a} is independently selected from the group consisting of: H; C₁₋₄ alkyl; C₃₋₆ cycloalkyl; —C(═O)(C₁₋₆ alkyl); —C(═O)O(C₁₋₆ alkyl); and —S(O)₁₋₂(C₁₋₆ alkyl);
each occurrence of R^{b} is independently selected from the group consisting of: R³¹; C₁₋₄ haloalkyl; —OH; oxo; —F; —N(R^{a})(R″); C₁₋₄ alkoxy; C₁₋₄ haloalkoxy; —C(═O)(C₁₋₄ alkyl); —C(═O)O(C₁₋₆ alkyl); —C(═O)OH, —C(═O)N(R″′)(R″″), —S(O)₁₋₂(C₁₋₆ alkyl); and cyano;
each occurrence of R^{c} is independently selected from the group consisting of: C₁₋₆ alkyl optionally substituted with from 1-2 independently selected R^{e}; C₃₋₆ cycloalkyl; C₁₋₄ haloalkyl; —OH; -halo; —NO₂; N₃; —N(R^{a})(R″); C₁₋₄ alkoxy; C₁₋₄ thioalkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{d} is independently selected from the group consisting of: C₁₋₆ alkyl optionally substituted with from 1-2 independently selected R^{e}; C₁₋₄ haloalkyl; —OH; —F; Cl; —N(R^{a})(R″); oxo; C₁₋₄ alkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{e} is independently selected from the group consisting of: —OH; —N(R^{a})(R″); C₁₋₄ alkoxy; C₁₋₄ haloalkoxy; —OC(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R″, R′″, and R″″ is independently selected from the group consisting of: H and C₁₋₆ alkyl; and
W is a peptide having the formula W′—R⁵, wherein W′ is a sequence of from 5-60 amino acids, and R⁵ is a C-terminal amino acid, amino acid ester, or amino acid amide that is optionally substituted with from 1-2 modifying groups (e.g., 1-2 groups selected from an acyl group and a PEG group).

In some embodiments, the compound has Formula (IIA):

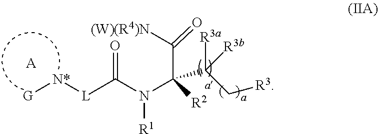

In some embodiments, the compound has Formula (I):

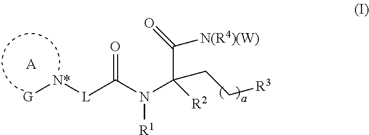

wherein:
ring A is:
(i) a saturated or unsaturated monocyclic ring that includes from 3-8 ring atoms (inclusive of G and the nitrogen atom labelled N*); or
(ii) a saturated or unsaturated bicyclic or tricyclic ring that includes from 6-14 ring atoms (inclusive of G and the nitrogen atom labelled N*), wherein:
   G is C(O), S(O), or SO₂; and
   the dotted, circular line connecting G and N* is a divalent group that includes from 1-6 ring atoms; wherein:
   (a) from 0-2 of the divalent group's ring atoms are ring heteroatoms, which are each independently selected from the group consisting of N, N(R^{a}), O, S, and SO₂; and
   (b) from 1-6 of the divalent group's ring atoms are ring carbon atoms, which are each independently selected from the group consisting of C, CH, CH₂, CR^{b}, C(R^{b})₂, and CHR^{b};
wherein:
(1) when ring A is a monocyclic ring, then each of the divalent group's 1-6 ring carbon atoms is independently selected from the group consisting of CH, CH₂, CR^{b}, C(R^{b})₂, and CHR^{b}; and
(2) when ring A is a bicyclic or tricyclic ring, then (A) or (B) applies:
   (A) two or three adjacent ring carbon atoms of the divalent group are each independently selected from the group consisting of C, CH, and, CR^{b} and are fused to a second ring that is selected from the group consisting of:
      (a) C₆₋₁₀ aryl optionally substituted with from 1-5 independently selected R^{c};
      (b) heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
      (c) C₃₋₁₀ cycloalkyl optionally substituted with from 1-4 independently selected R^{d};
      (d) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d}; or
   (B) one of the divalent group's ring carbon atoms is C and is spiro-fused to a second ring that is selected from the group consisting of:
      (a) C₃₋₁₀ cycloalkyl optionally substituted with from 1-4 independently selected R^{d};
      (b) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d};
L is:
(i) —(CH₂)ₘ—X¹—(CH₂)ₙ—X²—(CH₂)ₚ— (formula VIII), wherein m is from 1-6; n is from 0-2; and p is from 0-8;
(ii) —C(O)—(CH₂)ₙ—X³—(CH₂)ₚ— (formula IX), wherein n is from 0-2; and p is from 0-8;
(iii) —(CH₂)_{q}—, wherein q is from 1-10;
(iv) —C(O)—; or
(v) —(CH₂)ₘ—X¹—(CH₂)ₚ— (formula VIII), wherein m is from 1-6; and p is from 0-8;
X¹ is —C(O)—; —N(R′)C(O)—; —C(O)N(R′)—; or —N(R′)C(O)NR′)—; wherein each occurrence of R′ is independently selected from the group consisting of: H and C₁₋₄ alkyl;
X² is:
(i) —O—;
(ii) —S—;
(iii) —S(O)ₜ—, wherein t is 1 or 2;
(iv) —C(Q¹)(Q²)-, wherein each of Q¹ and Q² is independently selected from the group consisting of H and C₁₋₄ alkyl; or Q¹ and Q², together with the carbon atom to which each is attached forms C₃₋₁₀ cycloalkyl that is optionally substituted with from 1-4 independently selected R^{d} and optionally fused to phenyl;
(v) C₆₋₁₀ arylene optionally substituted with from 1-5 independently selected R^{c};
(vi) heteroarylene including from 5-10 ring atoms, wherein from 1-4 ring atoms are each independently selected from the group consisting of N, N(R^{a}), O, and S; and which is optionally substituted with from 1-5 independently selected R^{c};
(vii) C₃₋₁₀ cycloalkylene optionally substituted with from 1-4 independently selected R^{d};
(viii) heterocycloalkylene including from 3-10 ring atoms, wherein from 1-3 ring atoms are each independently selected from the group consisting of N(R^{a}), O, and S; and which is optionally substituted with from 1-5 independently selected R^{d};
(ix) C₂-C₄ alkenylene optionally substituted with from 1-2 R^{e}; or
(x) C₂-C₄ alkynylene optionally substituted with from 1-2 R^{e};
X³ is: —O—; —S—; or —S(O)ₜ—, wherein t is 1 or 2;
each of R¹, R², and R⁴ is independently selected from the group consisting of H and unsubstituted C₁₋₃ alkyl;
R³ is —C(O)OH, —C(O)OR³¹, or a carboxylic acid isostere; wherein R³¹ is:
(i) C₁₋₆ alkyl optionally substituted with from 1-2 independently selected R^{e};
(ii) —(C₀₋₃ alkylene)-C₃₋₁₀ cycloalkyl, wherein the cycloalkyl is optionally substituted with from 1-4 independently selected R^{d};
(iii) —(C₀₋₃ alkylene)-heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d};
(iv) —(C₀₋₃ alkylene)-C₆₋₁₀ aryl, wherein the aryl is optionally substituted with from 1-5 independently selected R^{c}; or
(v) —(C₀₋₃ alkylene)-heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
a is 0-5;
each occurrence of R^{a} is independently selected from the group consisting of: H; C₁₋₄ alkyl; C₃₋₆ cycloalkyl; —C(═O)(C₁₋₆ alkyl); —C(═O)O(C₁₋₆ alkyl); and —S(O)₁₋₂(C₁₋₆ alkyl);
each occurrence of R^{b} is independently selected from the group consisting of: R³¹; C₁₋₄ haloalkyl; —OH; oxo; —F; —N(R^{a})(R″); C₁₋₄ alkoxy; C₁₋₄ haloalkoxy; —C(═O)(C₁₋₄ alkyl); —C(═O)O(C₁₋₆ alkyl); —C(═O)OH, —C(═O)N(R″′)(R″″), —S(O)₁₋₂(C₁₋₆ alkyl); and cyano;
each occurrence of R^{c} is independently selected from the group consisting of: C₁₋₆ alkyl optionally substituted with from 1-2 independently selected R^{e}; C₃₋₆ cycloalkyl; C₁₋₄ haloalkyl; —OH; -halo; —NO₂; N₃; —N(R^{a})(R″); C₁₋₄ alkoxy; C₁₋₄ thioalkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{d} is independently selected from the group consisting of: C₁₋₆ alkyl optionally substituted with from 1-2 independently selected R^{e}; C₁₋₄ haloalkyl; —OH; —F; Cl; —N(R^{a})(R″); oxo; C₁₋₄ alkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{e} is independently selected from the group consisting of: —OH; —N(R^{a})(R″); C₁₋₄ alkoxy; C₁₋₄ haloalkoxy; —OC(═O)(C₁₋₆ alkyl); —C(═O)O(C₁₋₆ alkyl); —C(═O)OH, —C(═O)N(R″′)(R″″), —S(O)₁₋₂(C₁₋₆ alkyl); and cyano;
each occurrence of R″, R′″, and R″″ is independently selected from the group consisting of: H and C₁₋₆ alkyl; and
W is a peptide having the formula W′—R⁵, wherein W′ is a sequence of from 5-60 amino acids, and R⁵ is a C-terminal amino acid, amino acid ester, or amino acid amide that is optionally substituted with from 1-2 modifying groups (e.g., 1-2 groups selected from an acyl group and a PEG group).

In some embodiments, one or more of the compound provisions delineated herein (e.g., as delineated in the section below) apply.

### Compound Provisions

In some embodiments, the compound is other than CASRN 1401463-49-7; and/or CAS RN 1116465-43-0; and/or CAS RN 1614237-35-2, and/or CASRN 1614237-36-3; and/or CAS RN 1353718-57-6; and/or CAS Registry Number: 1083307-90-7, in which each of the registered chemical structures is incorporated herein by reference.

In some embodiments, the compound is not any of the compounds disclosed in Nacheva et al., Organic & Biomolecular Chemistry, 2012, 10 (38), 7840-7846, Chopra et al., Bioconjugate Chemistry, 2009, 20 (2), 231-240, and PCT application publication No. WO 2013/148579, each of which is incorporated by reference.

In some embodiments, ring A is other than unsubstituted maleimid-1-yl, i.e.:

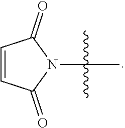 [SMILES: *N1C(=O)C=CC1=O]

In certain embodiments, when L is —(CH₂)₂—, —(CH₂)₅—C(O)NH—CH₂—, or —(CH₂)₂—C(O)NH—(CH₂)₆—, then ring A is other than unsubstituted maleimid-1-yl.

In certain embodiments, when L is —(CH₂)₂—, —(CH₂)₅—C(O)NH—CH₂—, or —(CH₂)₂—C(O)NH—(CH₂)₆—, and a is 1, then ring A is other than unsubstituted maleimid-1-yl.

In some embodiments, ring A is other than substituted maleimid-1-yl (e.g., other than 3,4-bis(2,4-difluorophenyl)-maleimid-1-yl, i.e.:

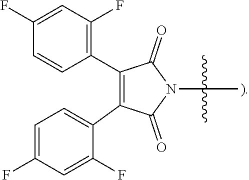 [SMILES: *N1C(=O)C(c2ccc(F)cc2F)=C(c2ccc(F)cc2F)C1=O]

In certain embodiments, when L is —(CH₂)₃—, then ring A is other than substituted maleimid-1-yl (e.g., other than 3,4-bis(2,4-difluorophenyl)-maleimid-1-yl).

In certain embodiments, when L is —(CH₂)₃—, and a is 1, then ring A is other than substituted maleimid-1-yl (e.g., other than 3,4-bis(2,4-difluorophenyl)-maleimid-1-yl).

In some embodiments, ring A is other than optionally substituted N-[2-(1,3-dioxo-1H-benz[de]isoquinolin-2(3H)-yl, (e.g., other than unsubstituted N-[2-(1,3-dioxo-1H-benz[de]isoquinolin-2(3H)-yl, i.e.:

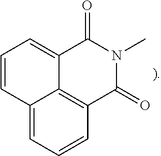 [SMILES: CN1C(=O)c2cccc3cccc(c23)C1=O]

In certain embodiments, when L is —(CH₂)₃—, then ring A is other than than optionally substituted N-[2-(1,3-dioxo-1H-benz[de]isoquinolin-2(3H)-yl, (e.g., other than unsubstituted N-[2-(1,3-dioxo-1H-benz[de]isoquinolin-2(3H)-yl).

In certain embodiments, when L is —(CH₂)₃—, and a is 0, then ring A is other than than optionally substituted N-[2-(1,3-dioxo-1H-benz[de]isoquinolin-2(3H)-yl, (e.g., unsubstituted N-[2-(1,3-dioxo-1H-benz[de]isoquinolin-2(3H)-yl).

In some embodiments, ring A is other than:
(i) unsubstituted maleimid-1-yl; and
(ii) substituted maleimid-1-yl (e.g., other than 3,4-bis(2,4-difluorophenyl)-maleimid-1-yl).

In some embodiments, ring A is other than:
(i) unsubstituted maleimid-1-yl;
(ii) substituted maleimid-1-yl (e.g., other than 3,4-bis(2,4-difluorophenyl)-maleimid-1-yl), and
(iii) optionally substituted N-[2-(1,3-dioxo-1H-benz[de]isoquinolin-2(3H)-yl, (e.g., other than unsubstituted N-[2-(1,3-dioxo-1H-benz[de]isoquinolin-2(3H)-yl).

In some embodiments:
(i) when L is —(CH₂)₂—, —(CH₂)₅—C(O)NH—CH₂—, or —(CH₂)₂—C(O)NH—(CH₂)₆—, then ring A is other than unsubstituted maleimid-1-yl; and
(ii) when L is —(CH₂)₃—, then ring A is other than substituted maleimid-1-yl (e.g, other than 3,4-bis(2,4-difluorophenyl)-maleimid-1-yl).

In some embodiments:
(i) when L is —(CH₂)₂—, —(CH₂)₅—C(O)NH—CH₂—, or —(CH₂)₂—C(O)NH—(CH₂)₆—, and a is 1, then ring A is other than unsubstituted maleimid-1-yl; and
(ii) when L is —(CH₂)₃—, and a is 1, then ring A is other than substituted maleimid-1-yl (e.g., other than 3,4-bis(2,4-difluorophenyl)-maleimid-1-yl).

In some embodiments:
(i) when L is —(CH₂)₂—, —(CH₂)₅—C(O)NH—CH₂—, or —(CH₂)₂—C(O)NH—(CH₂)₆—, then ring A is other than unsubstituted maleimid-1-yl;
(ii) when L is —(CH₂)₃—, then ring A is other than substituted maleimid-1-yl (e.g, other than 3,4-bis(2,4-difluorophenyl)-maleimid-1-yl); and
(iii) when L is —(CH₂)₃—, then ring A is other than optionally substituted N-[2-(1,3-dioxo-1H-benz[de]isoquinolin-2(3H)-yl, (e.g., other than unsubstituted N-[2-(1,3-dioxo-1H-benz[de]isoquinolin-2(3H)-yl).

In some embodiments:
(i) when L is —(CH₂)₂—, —(CH₂)₅—C(O)NH—CH₂—, or —(CH₂)₂—C(O)NH—(CH₂)₆—, and a is 1, then ring A is other than unsubstituted maleimid-1-yl;
(ii) when L is —(CH₂)₃—, and a is 1, then ring A is other than substituted maleimid-1-yl (e.g., other than 3,4-bis(2,4-difluorophenyl)-maleimid-1-yl); and
(iii) when L is —(CH₂)₃—, and a is 0, then ring A is other than optionally substituted N-[2-(1,3-dioxo-1H-benz[de]isoquinolin-2(3H)-yl, (e.g., unsubstituted N-[2-(1,3-dioxo-1H-benz[de]isoquinolin-2(3H)-yl.

In some embodiments, the first three amino acids in W′ must be glycine-threonine-phenylalanine (GTF).

In some embodiments, W′ must include 11 or more amino acids (e.g., 12 or more, 13 or more, 14 or more, 15 or more, 16 or more, 17 or more, 18 or more, 19 or more, 20 or more, 25 or more, 30 or more amino acids).

In some embodiments, the first three amino acids in W′ must be glycine-threonine-phenylalanine (GTF); and W′ must include 11 or more amino acids (e.g., 12 or more, 13 or more, 14 or more, 15 or more, 16 or more, 17 or more, 18 or more, 19 or more, 20 or more, 25 or more, 30 or more amino acids).

In certain of these embodiments, one or more of the following applies. Ring A is unsubstituted maleimid-1-yl, and L is —(CH₂)₂—, —(CH₂)₅—C(O)NH—CH₂—, or —(CH₂)₂—C(O)NH—(CH₂)₆— (optionally, a is 1). Ring A is substituted maleimid-1-yl (e.g., 3,4-bis(2,4-difluorophenyl)-maleimid-1-yl), and L is —(CH₂)₃— (optionally, a is 1). Ring A is optionally substituted N-[2-(1,3-dioxo-1H-benz[de]isoquinolin-2(3H)-yl, (e.g., unsubstituted N-[2-(1,3-dioxo-1H-benz[de]isoquinolin-2(3H)-yl) (optionally, a is 0).

In certain embodiments, when L is (CH₂)_{q}, q is not 3. In certain embodiments, when ring A is substituted with 2 R^{b} groups, the R^{b} groups are not both o,p-(di-fluoro)phenyl, or ring A is substituted with 2 o,p-(di-fluoro)phenyl groups, the first amino acid in the sequence W′ is not valine, or when ring A is substituted with 2 o,p-(di-fluoro)phenyl groups, L is not (CH₂)₃; or when L is (CH₂)₃, the first amino acid is the sequence W′ is not valine.

In certain embodiments, when L is —(CH₂)ₘ—X¹—(CH₂)ₙ—X²—(CH₂)ₚ—, m is 5, X¹ is —C(O)NH—, n and p are each zero, and X² is CQ¹Q², at least one of Q¹ and Q² is not H. In certain embodiments, when L is —(CH₂)ₘ—X¹—(CH₂)ₙ—X²—(CH₂)ₚ—, m is 5, X¹ is —C(O)NH—, n and p are each zero, and X² is CH₂, ring A is not —N-maleimide. or In certain embodiments, when L is —(CH₂)ₘ—X¹—(CH₂)ₙ—X²—(CH₂)ₚ—, m is 5, X¹ is —C(O)NH—, n and p are each zero, X² is CH₂, and ring A is —N-maleimide, the first amino acid is the sequence W′ is not glycine.

In certain embodiments, when L is —(CH₂)ₘ—X¹—(CH₂)ₙ—X²—(CH₂)ₚ—, m is 2, X¹ is —C(O)NH—, the sum of n and p is 5, and X² is CQ¹Q², at least one of Q¹ and Q² is not H. In certain embodiments, when L is —(CH₂)ₘ—X¹—(CH₂)ₙ—X²—(CH₂)ₚ—, m is 2, X¹ is —C(O)NH—, the sum of n and p is 5, and X² is CH₂, ring A is not —N-maleimide. In certain embodiments, when L is —(CH₂)ₘ—X¹—(CH₂)ₙ—X²—(CH₂)ₚ—, m is 2, X¹ is —C(O)NH—, the sum of n and p is 5, X² is CH₂, and ring A is —N-maleimide, the first amino acid in the sequence W′ is not alanine.

In certain embodiments, when L is (CH₂)_{q}, q is not 2; or when L is (CH₂)₂, ring A is not —N-maleimide; or when L is (CH₂)₂ and ring A is —N-maleimide, the first amino acid in the sequence W′ is not alanine.

### Ring A

In some embodiments, G is C(O). By way of non-limiting example, such compounds can include those having formula (I-A), (I-AA), or (I-AB):

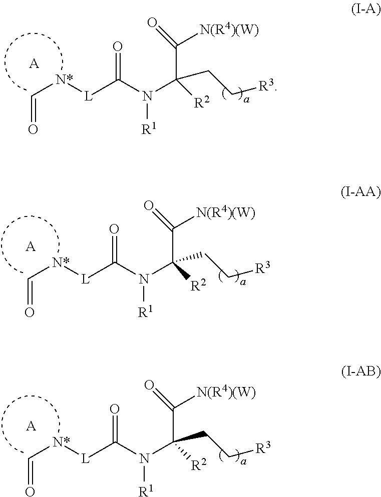

Further non-limiting examples include compounds having formula (IA-A), (IA-AA), or (IA-AB):

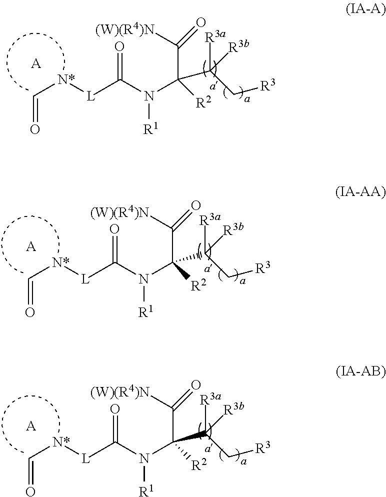

In some embodiments, G is S(O) or SO₂. By way of non-limiting example, such compounds can include those having formula (I-B), (I-BA), or (I-BB):

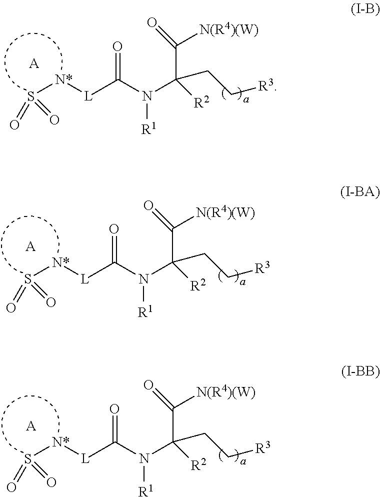

Further non-limiting examples include compounds having formula (IA-B), (IA-BA), or (IA-BB):

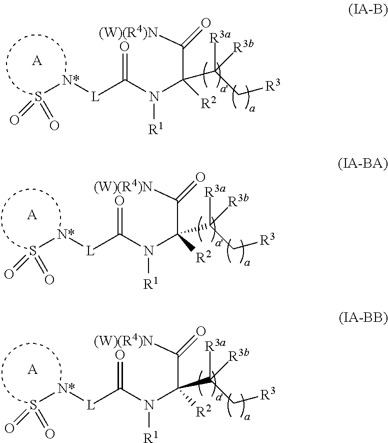

In some embodiments, ring A is a saturated or unsaturated monocyclic ring that includes from 3-8 (e.g., 4-8, 4-7, 5-6) ring atoms.

In certain of these embodiments, ring A is saturated. In other embodiments, ring A is unsaturated. In certain of these embodiments, ring A includes 4-7 ring atoms or 5-6 ring atoms.

In certain embodiments, ring A is a saturated monocyclic ring that includes from 3-8 (e.g., 4-8, 4-7, 5-6) ring atoms.

In certain embodiments, ring A is a saturated monocyclic ring that includes from 5-7 ring atoms.

In certain embodiments, ring A is a saturated monocyclic ring that includes from 5-6 ring atoms.

In certain embodiments, ring A has the following formula (III):
wherein:

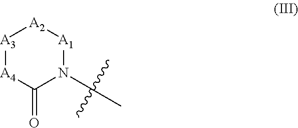

A₁ is a bond, A^{1A}-A^{1B}, C(O), CH₂, CHR^{b}, or C(R^{b})₂;
each of A^{1A} and A^{1B} is independently C(O), CH₂, CHR^{b}, or C(R^{b})₂;
A₂ is C(O), CH₂, CHR^{b}, or C(R^{b})₂;
A₃ is C(O), CH₂, CHR^{b}, or C(R^{b})₂; O; S; SO₂, or N(R^{a});
A₄ is C(O), CH₂, CHR^{b}, or C(R^{b})₂; O; S; or N(R^{a}); provided that A₃ and A₄ cannot both be O; S; or N(R^{a}); or a combination thereof.

In certain embodiments, ring A has formula (III):

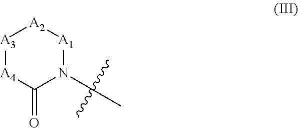

wherein:
A₁ is a bond, C(O), CH₂, CHR^{b}, or C(R^{b})₂;
A₂ is C(O), CH₂, CHR^{b}, or C(R^{b})₂;
A₃ is C(O), CH₂, CHR^{b}, or C(R^{b})₂; O; S; SO₂; or N(R^{a}); and
A₄ is CH₂, CHR^{b}, or C(R^{b})₂; O; S; or N(R^{a}); provided that A₃ and A₄ cannot both be O; S; or N(R^{a}); or a combination thereof.

In certain embodiments of formula (III), A₁ is other than a bond (i.e., A₁ is C(O), CH₂, CHR^{b}, or C(R^{b})₂), and ring A includes 6 ring atoms (i.e., ring A is a 6-membered ring).

In certain formula (III), 6-membered ring embodiments, A₁ is CH₂, CHR^{b}, or C(R^{b})₂. For example, A₁ can be CH₂. In other embodiments, A₁ is C(O).

In certain formula (III), 6-membered ring embodiments, A₂ is CH₂, CHR^{b}, or C(R^{b})₂. For example, A₂ can be CH₂.

In certain formula (III), 6-membered ring embodiments, A₃ is CH₂, CHR^{b}, or C(R^{b})₂. For example, A₃ can be CH₂. In other embodiments, A₃ is O; S; SO₂; or N(R^{a}).

In certain formula (III), 6-membered ring embodiments, A₄ is CH₂, CHR^{b}, or C(R^{b})₂. For example, A₄ can be CH₂. In other embodiments, A₄ is O; S; or N(R^{a}).

In certain formula (III), 6-membered ring embodiments, A₂, A₃ and A₄ are each independently selected from the group consisting of CH₂, CHR^{b}, or C(R^{b})₂.

In certain embodiments, one or two (e.g., one) of A₂, A₃ and A₄ are independently selected from the group consisting of CHR^{b} and C(R^{b})₂, and the other(s) is/are CH₂.

In certain formula (III), 6-membered ring embodiments, A₂, A₃ and A₄ are each CH₂.

In certain formula (III), 6-membered ring embodiments, A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₄ are both CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₃ are independently selected from the group consisting of CHR^{b} and C(R^{b})₂, and the other (where applicable) is CH₂. In other embodiments, A₂ and A₃ are both CH₂. In certain of these embodiments, A₄ is N(R^{a}) (e.g., A₄ can be NH). In other embodiments, A₄ is O.

In certain formula (III), 6-membered ring embodiments, A₁, A₂, A₃ and A₄ are each independently selected from the group consisting of CH₂, CHR^{b}, or C(R^{b})₂.

In certain formula (III), 6-membered ring embodiments, one or two (e.g., one) of A₁, A₂, A₃ and A₄ are independently selected from the group consisting of CHR^{b} and C(R^{b})₂, and the others are CH₂.

In certain formula (III), 6-membered ring embodiments, A₁, A₂, A₃ and A₄ are each CH₂.

In certain formula (III), 6-membered ring embodiments, A₁, A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₃ are both CH₂. In certain of these embodiments, A₁ is CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₁, A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₃ are both CH₂. In certain of these embodiments, A₁ is CH₂. In certain of these embodiments, A₄ is N(R^{a}) (e.g., A₄ can be NH). In other embodiments, A₄ is O.

In certain formula (III), 6-membered ring embodiments, A₁ is C(O); and A₂, A₃ and A₄ are each independently selected from the group consisting of CH₂, CHR^{b}, or C(R^{b})₂. In certain of these embodiments, one or two (e.g., one) of A₂, A₃ and A₄ are independently selected from the group consisting of CHR^{b} and C(R^{b})₂, and the other(s) is/are CH₂.

In certain formula (III), 6-membered ring embodiments, A₁ is C(O); and A₂, A₃ and A₄ are each CH₂.

In certain formula (III), 6-membered ring embodiments, A₁ is C(O); A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₄ are both CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₁ is C(O); A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₃ are independently selected from the group consisting of CHR^{b} and C(R^{b})₂, and the other (where applicable) is CH₂. In other embodiments, A₂ and A₃ are both CH₂. In certain of these embodiments, A₄ is N(R^{a}) (e.g., A₄ can be NH). In other embodiments, A₄ is O.

In certain embodiments of formula (III), A₁ is a bond, and ring A includes 5 ring atoms (i.e., ring A is a 5-membered ring), and ring A has formula (III-A):

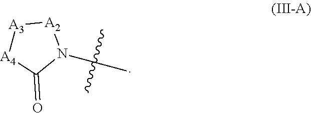

In certain formula (III-A) embodiments, A₂ is C(═O). In other embodiments, A₂ is CH₂, CHR^{b}, or C(R^{b})₂; for example, A₂ can be CH₂.

In certain formula (III-A) embodiments, A₃ is O; S; or N(R^{e}). For example, A₃ can be S. As another example, A₃ can be N(R^{a}) (e.g., A₃ can be NH).

In certain formula (III-A) embodiments, A₄ is CH₂, CHR^{b}, or C(R^{b})₂. For example, A₄ can be CHR^{b} or C(R^{b})₂. As another example, A₄ can be CH₂.

In certain formula (III-A) embodiments, A₂ is C(═O); A₃ is O; S; or N(R^{e}); and A₄ is CH₂, CHR^{b}, or C(R^{b})₂. In certain of these embodiments, A₃ is S. In other embodiments, A₃ is N(R^{a}) (e.g., A₃ is NH). In certain of these embodiments, A₄ is CH₂. In other embodiments, A₄ is CHR^{b} or C(R^{b})₂.

In certain formula (III-A) embodiments, A₂ is C(═O); and each of A₃ and A₄ is independently selected from CH₂, CHR^{b}, or C(R^{b})₂. In certain of these embodiments, each of A₃ and A₄ is CH₂. In other embodiments, one of A₃ and A₄ is CH₂, and the other of A₃ and A₄ is CHR^{b} or C(R^{b})₂. In still other embodiments, each of A₃ and A₄ is independently selected from CHR^{b} or C(R^{b})₂.

In certain formula (III-A) embodiments, A₂ is CH₂, CHR^{b}, or C(R^{b})₂, e.g., A₂ can be CH₂; and each of A₃ and A₄ is independently selected from CH₂, CHR^{b}, or C(R^{b})₂ (e.g, each of A₃ and A₄ is CH₂).

In certain of formula (III) embodiments, A₁ is A^{1A}-A^{1B}.

In certain of the foregoing formula (III) embodiments, each of A^{1A} and A^{1B} is independently CH₂, CHR^{b}, or C(R^{b})₂. As a non-limiting example, each of A^{1A} and A^{1B} can be independently CH₂.

In certain of formula (III) embodiments (when A₁ is A^{1A}-A^{1B}), A₂ is CH₂, CHR^{b}, or C(R^{b})₂. As a non-limiting example, A₂ can be CH₂.

In certain of formula (III) embodiments (when A₁ is A^{1A}-A^{1B}), A₃ is CH₂, CHR^{b}, or C(R^{b})₂. As a non-limiting example, A₃ can be CH₂.

In certain of formula (III) embodiments (when A₁ is A^{1A}-A^{1B}), A₄ is CH₂, CHR^{b}, or C(R^{b})₂. As a non-limiting example, A₄ is CH₂.

In certain of the foreging formula (III) and (III-A) embodiments, each occurrence of R^{b} is independently selected from the group consisting of:
(i) C₁₋₆ alkyl optionally substituted with from 1-2 independently selected R^{e};
(ii) —(C₀₋₃ alkylene)-C₆₋₁₀ aryl, wherein the aryl is optionally substituted with from 1-5 independently selected R^{c};
(iii) —(C₀₋₃ alkylene)-heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
(iv) —OH;
(v) —N(R^{a})(R″);
(vi) C₁₋₄ alkoxy; and
(vii) C₁₋₄ haloalkoxy; and
(viii) —F.

In certain of the formula (III) and (III-A) embodiments, each occurrence of R^{b} is independently selected from the group consisting of:
(i) C₁₋₆ alkyl;
(iv) —OH;
(v) —N(R^{a})(R″);
(vi) C₁₋₄ alkoxy;
(vii) C₁₋₄ haloalkoxy; and
(viii) —F.

In certain of the foreging formula (III) embodiments, each occurrence of R^{b} is independently selected from the group consisting of: C₁₋₆ alkyl optionally substituted with from 1-2 independently selected R^{e}; phenyl optionally substituted with from 1-5 independently selected R^{e}; —OH; —F; —N(R^{a})(R″); and C₁₋₄ alkoxy.

Non-limiting examples of ring A moities having formula (III) or (III-A) are delineated in tabular format in Table 1 below.

TABLE 1

| Example | A₁ | A₂ | A₃ | A₄ |
|---|---|---|---|---|
| III-1 | Bond | C(O) | S | CH2 |
| III-2 | CH2 | CH2 | CH2 | CH2 |
| III-3 | Bond | C(O) | NH | CH2 |
| III-4 | CH2 | CH2 | S | CH2 |
| III-5 | CH2 | CH2 | SO2 | CH2 |
| III-6 | CH2-CH2 | CH2 | CH2 | CH2 |
| III-7 | CH2 | CH2 | CH2 | C(O) |
| III-8 | CH2 | CH2 | 0 | CH2 |

In some embodiments, ring A is an unsaturated monocyclic ring that includes from 4-8 (e.g., 4-7, 5-6) ring atoms.

In certain embodiments, ring A is an unsaturated monocyclic ring that includes from 4-7 ring atoms.

In certain embodiments, ring A is an unsaturated monocyclic ring that includes from 5-6 ring atoms.

In certain embodiments, ring A is an unsaturated monocyclic ring that includes 6 ring atoms.

In certain embodiments, ring A is other than an unsaturated monocyclic ring that includes 5 ring atoms (e.g., other than maleimide, e.g, unsubstituted or di-substituted maleimide).

In certain of these embodiments, ring A has formula (IV):

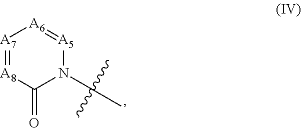

wherein:
each of A₆, and A₇ is independently selected from CH and CR^{b}; and
each of A₅ and A₈ is independently N, CH, or CR^{b}.

In certain of these embodiments, ring A has formula (IV):

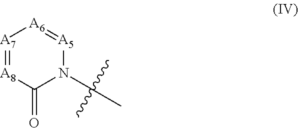

wherein:
each of A₅, A₆, and Az is independently selected from CH and CR^{b}; and
A₈ is N, CH, or CR^{b}.

In certain formula (IV) embodiments, A₅, A₆, A₇, and A₈ are each independently selected from CH and CR^{b}. For example, one or two of A₅, A₆, A₇, and A₈ are an independently selected CR^{b}, and the others are CH. As another example, A₅, A₆, A₇, and A₈ are each CH.

In other formula (IV) embodiments, A₅, A₆, and A₇ are each independently selected from CH and CR^{b}, and A₈ is N. For example, one or two of A₅, A₆, and A₇ are an independently selected CR^{b}, and the others are CH. As another example, A₅, A₆, and A₇ are each CH.

In still other formula (IV) embodiments, A₅ is N; and each of A₆, A₇, and A₈ is independently selected from CH and CR^{b} (e.g., one of A₆, A₇, and A₈ is CR^{b} (e.g., C—OH).

In certain of the foregoing formula (IV) embodiments, each occurrence of R^{b} is independently selected from the group consisting of: R³¹; C₁₋₄ haloalkyl; —OH; —N(R^{a})(R″); C₁₋₄ alkoxy; and C₁₋₄ haloalkoxy.

In certain of the foregoing formula (IV) embodiments, each occurrence of R^{b} is independently selected from the group consisting of: R³¹; —OH; —N(R^{a})(R″); C₁₋₄ alkoxy; and C₁₋₄ haloalkoxy.

In certain of the foregoing formula (IV) embodiments, each occurrence of R^{b} is independently selected from the group consisting of:
(i) C₁₋₆ alkyl optionally substituted with from 1-2 independently selected R^{e};
(ii) —(C₀₋₃ alkylene)-C₆₋₁₀ aryl, wherein the aryl is optionally substituted with from 1-5 independently selected R^{c};
(iii) —(C₀₋₃ alkylene)-heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
(iv) —OH;
(v) —N(R^{a})(R″);
(vi) C₁₋₄ alkoxy;
(vii) C₁₋₄ haloalkoxy; and
(viii) —F.

In certain of these embodiments, ring A has formula (V):

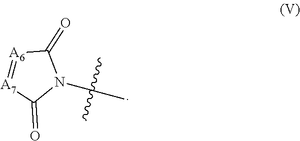

wherein:
each of A₆ and A₇ is independently selected from CH and CR^{b}; and
A₈ is N, CH, or CR^{b}.

In other formula (V) embodiments, A₆ and A₇ are each CH. In other formula (IV) embodiments, A₆ and A₇ cannot both be CH.

In certain formula (V) embodiments, one of A₆ and A₇ is CR^{b}, the other of A₆ and A₇ is CH.

In other formula (IV) embodiments, A₆ and A₇ are each an independently selected CR^{b}. In other formula (IV) embodiments, A₆ and A₇ cannot both be an independently selected CR^{b}. In certain of these embodiments, when A₆ and A₇ are each an independently selected CR^{b}, then both Rb substituents cannot be C₆₋₁₀ aryl (e.g., phenyl) substituted with from 1-5 independently selected R^{e}. As another example, both R^{b} substituents cannot be C₆₋₁₀ aryl (e.g., phenyl) substituted with from 1-3 or 1-2 independently selected R^{c}. For example, both R^{b} substituents cannot be C₆₋₁₀ aryl (e.g., phenyl), substituted with from 1-5 independently halo (e.g., fluoro). As another example, both R^{b} substituents cannot be C₆₋₁₀ aryl (e.g., phenyl), substituted with from 1-3 or 1-2 independently selected halo (e.g, fluoro). For example, both both R^{b} substituents cannot be difluorophenyl, e.g., o,p-(di-fluoro)phenyl.

In some embodiments, ring A is a saturated or unsaturated bicyclic or tricyclic ring that includes from 7-9 (e.g., 7-8, e.g, 7) ring atoms.

In certain of these embodiments, (A) applies (e.g., three adjacent ring carbon atoms of the divalent group are each independently selected from the group consisting of C, CH, CR^{b}, CH₂, C(R^{b})H, C(R^{b})₂ and are fused to a second ring).

In certain of these embodiments, ring A has the following formula:

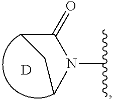

wherein D is:
(c) C₃₋₁₀ cycloalkyl optionally substituted with from 1-4 independently selected R^{d}; or
(d) heterocyclyl including from 4-10 ring atoms, wherein from 1-3 ring atoms are each independently selected from the group consisting of N(R^{a}), O, and S, wherein the heterocyclyl is optionally substituted with from 1-4 independently selected R^{d}.

As non-limiting examples of the foregoing embodiments, ring A can be:

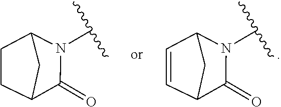 [SMILES: *N1C(=O)C2CCC1C2 ; *N1C(=O)C2C=CC1C2]

In some embodiments, ring A is a saturated or unsaturated bicyclic or tricyclic ring that includes from 8-10 ring atoms.

In certain of these embodiments, ring A is saturated. In other embodiments, ring A is unsaturated.

In certain of these embodiments, (A) applies (e.g., two adjacent ring carbon atoms of the divalent group are each independently selected from the group consisting of C, CH, and, CR^{b} and are fused to a second ring). In other embodiments, (B) applies.

In certain embodiments, ring A is an unsaturated bicyclic or tricyclic ring that includes from 8-10 ring atoms. In certain of these embodiments, (A) applies. In certain of these embodiments, ring A is optionally further substituted with 1 oxo.

In certain embodiments, (A) applies, ring A is optionally further substituted with 1 oxo, and ring A has formula (VI):

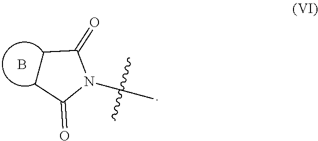

In certain of these embodiments, fused ring B is fused C₆₋₁₀ aryl optionally substituted with from 1-5 independently selected R^{c}. For example, fused ring B can be fused phenyl optionally substituted with from 1-5 independently selected R^{e}. As a further example, ring A can have formula (VI-A):

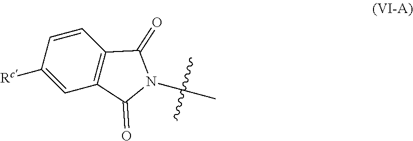

wherein R^{c′} is H or R^{e} (e.g., C₁₋₆ alkyl, e.g., CH₃).

In certain of the formula (VI) embodiments, ring B is fused C₁₀ aryl optionally substituted with from 1-5 independently selected R^{e} (e.g., unsubstituted).

In certain of the formula (VI) embodiments, ring B is fused heteroaryl including from 5-10 (e.g., 6) ring atoms, wherein from 1-4 (e.g., 1) ring atoms are each independently selected from the group consisting of N, N(R^{a}), O, and S, wherein the heteroaryl is optionally substituted with from 1-3 independently selected R^{c}.

In certain embodiments, ring A is selected from one of the follows:

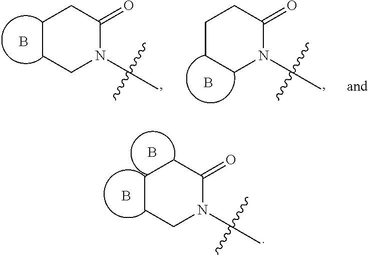

In certain of the foregoing embodiments, each fused ring B is independently selected from:
fused heteroaryl including from 5-10 (e.g., 6) ring atoms, wherein from 1-4 (e.g., 1) ring atoms are each independently selected from the group consisting of N, N(R^{a}), O, and S, wherein the heteroaryl is optionally substituted with from 1-3 independently selected R^{c}; and
fused C₆₋₁₀ (e.g., C₆) aryl optionally substituted with from 1-5 (e.g., from 1-2) independently selected R^{e}.

As a non-limiting example, each fused ring B can be independently a fused C₆₋₁₀ (e.g., C₆) aryl optionally substituted with from 1-5 (e.g., from 1-2) independently selected R^{c}.

In certain embodiments, ring A is a saturated bicyclic or tricyclic ring that includes from 8-10 ringatoms. In certain of these embodiments, (A) applies. In other embodiments, (B) applies. In certain of these embodiments, ring A is optionally further substituted with 1 oxo.

In certain embodiments, (A) applies, ring A is optionally further substituted with 1 oxo, and ring A has formula (VI):

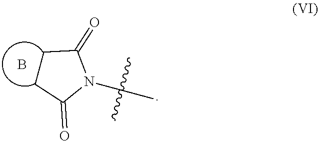

In certain of these embodiments, fused ring B is fused C₃₋₁₀ cycloalkyl optionally substituted with from 1-4 independently selected R^{d}. For example, fused ring B can be fused C₃₋₆ cycloalkyl (e.g., C₃₋₄ cycloalkyl) optionally substituted with from 1-4 independently selected R^{d} (e.g., R^{d} can be C₁₋₃ alkyl).

In other embodiments, (B) applies, ring A is optionally further substituted with 1 oxo, and ring A has formula (VII):

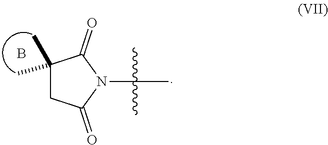

In certain of these embodiments, the spiro-fused ring B is spiro-fused C₃₋₁₀ cycloalkyl optionally substituted with from 1-4 independently selected R^{d}. For example, the spiro-fused ring B is spiro-fused C₃₋₆ cycloalkyl (e.g., C₃₋₄ cycloalkyl) optionally substituted with from 1-4 independently selected R^{d} (e.g., R^{d} can be C₁₋₃ alkyl).

### Variable L

In some embodiments, L has formula (VIII): —(CH₂)ₘ—X¹—(CH₂)ₙ—X²—(CH₂)ₚ—.

In certain embodiments of formula (VIII), m is from 2-6 (e.g., m can be 2).

In certain embodiments of formula (VIII), m is 1.

In certain embodiments of formula (VIII), X¹ is —N(R′)C(O)— (e.g., X¹ can be —N(H)C(O)—).

In certain embodiments of formula (VIII), n+p≥2. For example, n+p=2 (e.g., each of n and p is 1). As another example, n+p>2 (e.g., n+p=3, 4, 5, 6, 7, 8, 9, or 10).

In another embodiment, n+p<2.

In still other embodiments, n+p=0.

In certain embodiments of formula (VIII), X² is: —O—; —S—; or —S(O)ₜ—, wherein t is 1 or 2. For example, X² can be —S—.

In certain embodiments of formula (VIII), X² is —C(Q¹)(Q²)-.

In certain of these embodiments, each of Q¹ and Q² is independently selected from the group consisting of H and C₁₋₄ alkyl. For example, one of Q¹ and Q² can be H, and the other of Q¹ and Q² can be C₁₋₄ alkyl (e.g., CH₃). For example, Q¹ can be H; and Q² can be C₁₋₄ alkyl. As another example, each of Q¹ and Q² can be H. As a further example, each of Q¹ and Q² can be an independently selected C₁₋₄ alkyl.

In other embodiments, Q¹ and Q², together with the carbon atom to which each is attached forms C₃₋₁₀ cycloalkyl that is optionally substituted with from 1-4 independently selected R^{d} and optionally fused to phenyl. For example, Q¹ and Q², together with the carbon atom to which each is attached forms C₃₋₆ cycloalkyl that is optionally substituted with from 1-4 independently selected R^{d} and optionally fused to phenyl.

In certain of the foregoing embodiments, Q¹ and Q², together with the carbon atom to which each is attached forms C₄₋₆ (e.g., C₄) cycloalkyl that is optionally substituted with from 1-2 independently selected R^{d}.

In certain embodiments of formula (VIII), X² is:
(v) C₆₋₁₀ arylene optionally substituted with from 1-5 independently selected R^{c};
(vi) heteroarylene including from 5-10 ring atoms, wherein from 1-4 ring atoms are each independently selected from the group consisting of N, N(R^{a}), O, and S; and which is optionally substituted with from 1-5 independently selected R^{c};
(vii) C₃₋₁₀ cycloalkylene optionally substituted with from 1-4 independently selected R^{d}; or
(viii) heterocycloalkylene including from 3-10 ring atoms, wherein from 1-3 ring atoms are each independently selected from the group consisting of N(R^{a}), O, and S; and which is optionally substituted with from 1-5 independently selected R^{d}.

For example, X² can be:
(v) C₆₋₁₀ arylene optionally substituted with from 1-5 independently selected R^{c}; or
(vii) C₃₋₁₀ cycloalkylene optionally substituted with from 1-4 independently selected R^{d}.

As another example, X² can be:
(v) phenylene optionally substituted with from 1-5 (e.g., 1-3, 1-2, 1) independently selected R^{c}; or
(vii) C₃₋₆ cycloalkylene (e.g., C₃₋₄ cycloalkylene) optionally substituted with from 1-4 independently selected R^{d} (e.g., R^{d} can be C₁₋₃ alkyl).

In certain embodiments of formula (VIII), X² is (viii) heterocycloalkylene including from 3-10 ring atoms, wherein from 1-3 ring atoms are each independently selected from the group consisting of N(R^{a}), O, and S; and which is optionally substituted with from 1-5 independently selected R^{d}.

In certain embodiments of formula (VIII), X² is (viii) heterocycloalkylene including from 4-6 (e.g., 4) ring atoms, wherein from 1-3 (e.g., 1) ring atoms are each independently selected from the group consisting of N(R^{a}), O, and S; and which is optionally substituted with from 1-5 independently selected R^{d} (e.g., X² can be

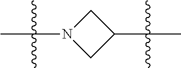 [SMILES: *C1CN(*)C1]

In some embodiments, L has formula (IX): L has formula (IX): —C(O)—(CH₂)ₙ—X³—(CH₂)ₚ—. In certain of these embodiments, n+p≥2. For example, n+p=2 (e.g., each of n and p is 1). As another example, n+p>2. In certain of these embodiments, X³ is —S—.

### Non-Limiting L Combinations

[1] In certain embodiments of formula (VIII):
m is from 2-6;
n+p≥2; and
X² is: —O—; —S—; or —S(O)ₜ—.

In certain of these embodiments, m is 2.

In certain of these embodiments, X¹ is —N(R′)C(O)— (e.g., —N(H)C(O)—).

In certain of these embodiments, n+p>2.

In certain of these embodiments, n+p=2 (e.g., each of n and p is 1).

In certain of these embodiments X² is —S—.

In certain of these embodiments, m is 2-6 (e.g., 2), X¹ is —N(R′)C(O)— (e.g., —N(H)C(O)—), and n+p>2.

In certain of these embodiments, m is 2-6 (e.g., 2), X¹ is —N(R′)C(O)— (e.g., —N(H)C(O)—), and n+p>2, and X² is —S—.

In certain of these embodiments, m is 2-6 (e.g., 2), X¹ is —N(R′)C(O)— (e.g., —N(H)C(O)—), and n+p=2 (e.g., each of n and p is 1).

In certain of these embodiments, m is 2-6 (e.g., 2), X¹ is —N(R′)C(O)— (e.g., —N(H)C(O)—), and n+p=2 (e.g., each of n and p is 1), and X² is —S—.

For example, L can be —CH₂CH₂NHC(O)CH₂SCH₂—.
[2] In certain embodiments of formula (VIII):
m is from 2-6;
n+p<2; and
X² is:
(i) —C(Q¹)(Q²)-, wherein each of Q¹ and Q² is an independently selected C₁₋₄ alkyl; or Q¹ and Q², together with the carbon atom to which each is attached forms C₃₋₁₀ cycloalkyl that is optionally substituted with from 1-4 independently selected R^{d} and optionally fused to phenyl.;
(v) C₆₋₁₀ arylene optionally substituted with from 1-5 independently selected R^{c};
(vi) heteroarylene including from 5-10 ring atoms, wherein from 1-4 ring atoms are each independently selected from the group consisting of N, N(R^{a}), O, and S; and which is optionally substituted with from 1-5 independently selected R^{c};
(vii) C₃₋₁₀ cycloalkylene optionally substituted with from 1-4 independently selected R^{d}; or
(viii) heterocycloalkylene including from 3-10 ring atoms, wherein from 1-3 ring atoms are each independently selected from the group consisting of N(R^{a}), O, and S; and which is optionally substituted with from 1-5 independently selected R^{d}.

In certain of these embodiments, m is 2.

In certain of these embodiments, X¹ is —N(R′)C(O)— (e.g., —N(H)C(O)—).

In certain of these embodiments, n+p=0.

In certain of these embodiments, X² is —C(Q¹)(Q²)-.

In certain of these embodiments, each of Q¹ and Q² is H.

In certain of these embodiments, Q¹ is C₁₋₄ alkyl.

In certain of these embodiments, Q² is C₁₋₄ alkyl.

In certain of these embodiments, each of Q¹ and Q² is an independently selected C₁₋₄ alkyl (e.g., CH₃).

In certain of these embodiments, Q¹ and Q², together with the carbon atom to which each is attached forms C₃₋₆ cycloalkyl that is optionally substituted with from 1-4 independently selected R^{d} and optionally fused to phenyl.

In certain of these embodiments, X² is:
(v) C₆₋₁₀ arylene optionally substituted with from 1-5 independently selected R^{c}; or
(vii) C₃₋₁₀ cycloalkylene optionally substituted with from 1-4 independently selected R^{d}.

In certain of these embodiments, X² is (viii) heterocycloalkylene including from 4-6 (e.g., 4) ring atoms, wherein from 1-3 (e.g., 1) ring atoms are each independently selected from the group consisting of N(R^{a}), O, and S; and which is optionally substituted with from 1-5 independently selected R^{d} (e.g., X² can be

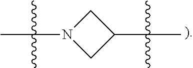 [SMILES: *C1CN(*)C1]

In certain of these embodiments, m is 2-6 (e.g., 2), X¹ is —N(R′)C(O)— (e.g., —N(H)C(O)—), and n+p=0.

In certain of these embodiments, m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl.

In certain of these embodiments, m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃).

In certain of these embodiments, m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.
[3] In certain embodiments of formula (IX): n+p=2 (e.g., each of n and p is 1), and X³ is —S—.
[4] In certain embodiments of formula (VIII):
m is 1;
n+p≤2; and
X² is: —O—; —S—; —S(O)ₜ—, or C(Q¹)(Q²).

In certain of these embodiments, n+p=2.

In certain of these embodiments, X² is —S—.

In certain of these embodiments, n+p<2 (e.g., n+p=0; or n+p=1).

In certain of these foregoing embodiments, X² is C (Q¹)(Q²).

In certain of these embodiments, each of Q¹ and Q² is H.

In certain of these embodiments, each of Q¹ and Q² is C₁₋₄ alkyl.

In certain of these embodiments, Q¹ is H; and Q² is C₁₋₄ alkyl.

Variables R¹, R², R^{2′}, and R⁴

In some embodiments, R¹ is H. In some embodiments, R² is H. In some embodiments, R⁴ is H.

In some embodiments, two of R¹, R², and R⁴ are H.

In some embodiments, each of R¹, R², and R⁴ is H.

In some embodiments, R² is unsubstituted C₁₋₃ alkyl (e.g., methyl).

In some embodiments, R¹ and R⁴ is H.

In some embodiments, R^{2′} is as defined according to (AA).

In certain embodiments, a′ is 1.

In certain of these embodiments, each of R^{3a} and R^{3b} is H.

In certain of these embodiments, each of R^{3a} and R^{3b} is C₁₋₃ alkyl (e.g., methyl).

In other embodiments, a′ is 0.

In some embodiments, R³ is —C(O)OH. In other embodiments, R³ is a carboxylic acid isostere. Non-limiting examples of carboxylic acid isosteres include noncarbon acyclic acids such as phosphonic/phosphinic and sulfonic/sulfinic acids and sulfonamides; modified carbon-based acids such as hydroxamic acids, hydroxamic esters, acylureas, acyl sulfonamides, and sulfonyl ureas; optionally substituted heteroaryl and heterocyclyl-based isosteres, such as those based on optionally substituted tetrazole, oxadiazol-5(4H)-thione, thiadiazol-5(4H)-one, oxathiadiazole and oxidized forms thereof, isoxazole, thiazolidinedione, oxazolidinedione, tetramic acid, and derivatives thereof; and optionally substituted cycloalkyl and aryl-based isosteres, such as those based on optionally substituted cyclopentane-1,3-dione, phenol and squaric acid and derivatives thereof (e.g, tetrazolyl or amide). In certain embodiments, R³ is an amide. In certain embodiments, R³ is an optionally substituted tetrazolyl (e.g., unsubstituted tetrazolyl). In certain embodiments, R³ is an optionally substituted isoxazolyl (e.g., isoxazolyl substituted with hydroxy

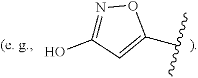 [SMILES: *c1cc(O)no1]

In some embodiments, a is 1-5 (e.g., 1-4, 1-3, 1-2). In some embodiments, a is 0. In some embodiments, a is 0, 1, or 2. In certain embodiments, a is 1. In other embodiments, a is 2. In still other embodiments, a is 0. In some embodiments, a is 0, 1, 2, or 3 (e.g., a=0; or a=1; or a=2).

### Variables W, W′, and R⁵

W is a peptide having the formula W′—R⁵, wherein W′ is a sequence of from 5-60 amino acids, and R⁵ is a C-terminal amino acid, amino acid ester, or amino acid amide that is optionally substituted with from 1-2 modifying groups (e.g., 1-2 groups selected from an acyl group and a PEG group). As used herein, “peptide” refers to a chain of amino acid monomers (sometimes termed “residues”) linked by peptide (amide) bonds (or an isosteric replacement thereof), which have an N-terminal (N—R⁴ in formulae (IAA), (IA), or (I) and a C-terminal amino acid residue (R⁵) at each of the ends of the peptide. The term peptide also includes modified peptides, including, e.g., any one or more of the modifications described herein.

### Constituent Amino Acids and Modifications Thereof

In some embodiments, W includes one or more naturally occurring amino acids found, e.g., in polypeptides and/or proteins produced by living organisms, such as Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H).

In some embodiments, W includes one or more independently selected modifications that occur present in so-called modified peptides. Such modifications include, but not limited to: (i) the incorporation of lactam-bridge; (ii) head-to-tail cyclization; (iii) one or more alternative or non-naturally occurring (D or L) amino acids, such as synthetic non-native amino acids, substituted amino acids, and D-amino acids; (iv) peptide bond replacements; (v) targeting groups; and the like. In certain embodiments, W includes one modification in either the W′ or R⁵ component. In other embodiments, W includes more than one independently selected modification (e.g., 2 independently selected modifications, 3 independently selected modifications, 4 independently selected modifications, 5 independently selected modifications, 6 independently selected modifications, 7 independently selected modifications, 8 independently selected modifications, 9 independently selected modifications, or 10 independently selected modifications that occur in the W′ and/or R⁵ component (e.g., in the W′ component only; or in the R⁵ component only; or in both the W′ and R⁵ components).

Non-limiting examples of alternative or non-naturally amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof (each which can be, where appropriate can each independently be D or L amino acids).

Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

Other non-limiting examples of modifications and/or moieties include PEGylation; glycosylation; HESylation; ELPylation; lipidation; acetylation; amidation; end-capping modifications (e.g., amidation at the C-terminus, introduction of C-terminal alcohol, aldehyde, ester, and thioester moieties); cyano groups; phosphorylation; cyclization, conjugation with targeting moieties and/or agents that increase retention in the body (e.g, agents such as cellulose, fatty acids, polyethylene glycol (PEG) or combinations thereof); incorporation of retro-inverso peptide motif (i.e., a peptide with a reversal of the direction of the peptide bond on at least one position);

In certain embodiments, W includes only naturally occurring amino acids. In other embodiments, W includes only alternative or non-naturally occurring amino acids. In still other embodiments, W includes one or more naturally occurring amino acids and one or more alternative or non-naturally occurring amino acids. In certain of the foregoing embodiments, W includes only L amino acids; or W includes both D and L amino acids; or W includes only D amino acids. While not wishing to be bound by theory, it is believed that the incorporation of D amino acids can confer enhanced in vivo or intracellular stability to the compounds described herein.

In some embodiments, W includes amino acid residues each of Formula XAA:

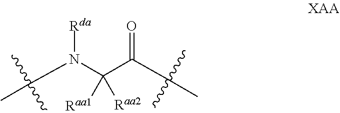

wherein each of R^{aa1} and R^{aa2} is independently selected from:
(a) H;
(b) C₁₋₆ alkyl, which is optionally substituted with from 1-3 R^{ba}.
(c) (C₀₋₃ alkylene)-C₃₋₁₀ cycloalkyl, which is optionally substituted with from 1-4 R^{ba};
(d) (C₀₋₃ alkylene)-heterocyclyl, wherein the heterocyclyl includes from 3-10 ring atoms, wherein from 1-3 ring atoms are heteroatoms, each independently selected from the group consisting of N(R^{da}), O, and S, and wherein one or more of the heterocyclyl ring carbon atoms are optionally substituted with from 1-4 independently selected R^{ba};
(e) (C₀₋₃ alkylene)-C₆-C₁₀ aryl, which is optionally substituted with from 1-4 R^{ca};
(f) (C₀₋₃ alkylene)-heteroaryl, wherein the heteroaryl includes from 5-10 ring atoms, wherein from 1-4 ring atoms are heteroatoms, each independently selected from the group consisting of N, N(R^{da}), O, and S, and wherein one or more of the heteroaryl ring carbon atoms are optionally substituted with from 1-4 independently selected R^{ca};
OR
(g) R^{aa1} and R^{da}, in the —C(═O)CR^{aa1}(R^{aa2})N(R^{da})— group, combine to form a ring including from 5-8 ring atoms, wherein the ring includes: (a) from 1-6 ring carbon atoms (in addition to CR^{aa1}(R^{aa2})), each of which is substituted with from 1-2 substituents independently selected from H and C₁₋₃ alkyl; and (b) from 0-3 ring heteroatoms (in addition to the nitrogen atom attached to R^{da}), which are each independently selected from the group consisting of N(R^{da}), O, and S;
each occurrence of R^{ba} is selected from the group consisting of —C(═O)(OH); —C(═O)(C₂₋₂₀ alkyl); —C(═O)NR′R″; —NHC(═NR′)NR′R″; —C(═O)O(C₂₋₂₀ alkyl); —S(O)₀₋₂(C₁₋₆ alkyl); oxo; F; C₁₋₁₀ alkoxy; C₁₋₁₀ haloalkoxy; azido; —N(R^{ga})(R^{ha});
each occurrence of R^{ca} is independently selected from the group consisting of: —OH; —SH; —F; —Cl; —Br; —NR′R″; C₁₋₄ alkyl; C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₀₋₂(C₁₋₄ alkyl); cyano; —NR′C(═NR′)NR′R″; C₆₋₁₀ aryl optionally substituted with 1-4 substituents independently selected from halo, C₁₋₄ alkyl, and C₁₋₄ haloalkyl; and C₃₋₆ cycloalkyl optionally substituted with from 1-4 independently selected C₁₋₄ alkyl; each occurrence of R^{da} is independently selected from the group consisting of: H, C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{aa1} and R^{da}, in the —C(═O)CR^{aa1}(R^{aa2})N(R^{da})— group, combine to form a ring including from 5-8 ring atoms as defined above;
each occurrence of R^{ga} and R^{ha} is independently selected from thr group consisting of H; C₁₋₄ alkyl; —C(═O)O(C₂₋₂₀ alkyl); and —S(O)₁₋₂(C₁₋₆ alkyl); and
each occurrence of R′ and R″ is independently selected from the group consisting of: H and C₁₋₄ alkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R′ and R″), which are each independently selected from the group consisting of N (H), O, and S;
or a C-terminal amino acid or an amino acid ester or an amino acid amide thereof.

In some embodiments, W includes 25-45 amino acid residues of Formula XAA, wherein R^{aa2} is H; or a C-terminal amino acid or an amino acid ester or an amino acid amide thereof.

In certain of these embodiments, R^{aa1} is selected from the group consisting of:
H;
C₁-C₆ alkyl optionally substituted with 1-2 substituents selected from —NH₂, —OH, —SH, —SMe, —NH(C═NH)NH₂, CO₂H, and CO₂NH₂;
(C₁-C₆ alkylene)-phenyl, optionally substituted with 1-2 —OH;
(C₁-C₆ alkylene)-indolyl; and
(C₁-C₆ alkylene)-imidazolyl; or
R^{aa1} and R^{da}, in the —CH(R^{aa1})N(R^{da})— group, combine to form a pyrrolidine ring.

As non-limiting examples of the foregoing, W can include from 25-45 naturally occurring amino acids found, e.g., in polypeptides and/or proteins produced by living organisms, such as Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H); or amino acid ester or amino acid amide thereof.

In certain embodiments, W includes from 1-2 (e.g., 1) amino acid residue of Formula XAA, wherein R^{aa2} is H; R^{aa1} is: C₁₋₆ alkyl, which is substituted with from 1-2 (e.g., 1) R^{ba}, and
each occurrence of R^{ba} is independently selected from:
—N(R^{ga})(R^{ha});
or a C-terminal amino acid or an amino acid ester or an amino acid amide thereof.

As non-limiting examples of the foregoing, W can include an amino acid residue selected from:

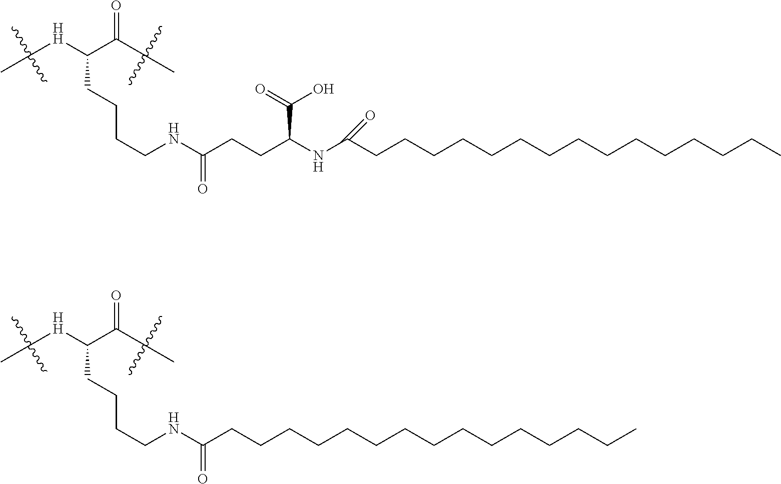

or a C-terminal amino acid or an amino acid ester or an amino acid amide thereof.

In certain embodiments, W includes from 1-2 (e.g., 1) amino acid residue of Formula XAA, wherein R^{aa2} is C₁₋₃ alkyl (e.g., methyl); and R^{aa1} is other than H;
or a C-terminal amino acid or an amino acid ester or an amino acid amide thereof.

Non-limiting examples include:

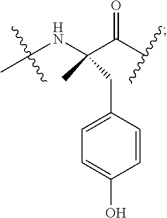 [SMILES: *N[C@@](C)(Cc1ccc(O)cc1)C(*)=O]

or a C-terminal amino acid or an amino acid ester or an amino acid amide thereof.

### Variable W′

In some embodiments, W′ is a sequence of from 20-60 (e.g., 20-55, 20-50, 20-45, 20-40, 20-30, 20-35) amino acids.

In some embodiments, W′ is a sequence of from 30-60 (e.g., 30-55, 30-50, 30-45, 30-40) amino acids. In certain embodiments, W′ is a sequence of from 25-45 (e.g., 30-45) amino acids. In certain embodiments, W′ is a sequence of from 30-50 amino acids. For example, W′ can be a sequence of from 30-45 amino acids or from 30-40 amino acids. In certain embodiments, W′ can be a sequence of 34, 35, 36, 37, 38, or 39 amino acids; e.g., 36 or 37 amino acids; e.g., 36 amino acids. In certain embodiments, W′ can be a sequence of 25-28 amino acids; e.g., 26 or 27 amino acids.

### Variable R⁵

In some embodiments, R⁵ is a C-terminal amino acid amide that is optionally substituted with from 1-2 modifying groups. In other embodiments, R⁵ is a C-terminal amino acid that is optionally substituted with from 1-2 modifying groups.

In certain embodiments, R⁵ is a C-terminal lysyl residue, e.g., a lysyl amide residue that is optionally substituted with from 1-2 modifying groups. For example, R⁵ can be a C-terminal L-lysyl amide residue that is optionally substituted with from 1-2 modifying groups. In certain embodiments, R⁵ has formula (XI):

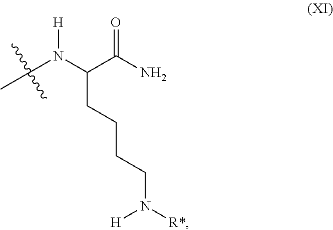

wherein R* is H or a modifying group. In certain of these embodiments, formula (XI) is L. In other embodiments, formula (XI) is D.

In certain embodiments, R* is H.

In certain embodiments, the modifying group (e.g., R*) is an acyl group. For example, the acyl group can be a C₂₋₃₀ (e.g., C₂₋₂₀, C₂₋₁₀, C₂₋₆) acyl group that is optionally substituted with from 1-2 independently selected R^{f}. Each occurrence of R^{f} is selected from the group consisting of —C(═O)(OH); —C(═O)(C₂₋₂₀ alkyl); —C(═O)O(C₂₋₂₀ alkyl); and —S(O)₁₋₂(C₁₋₆ alkyl); oxo; F; C₁₋₁₀ alkoxy; C₁₋₁₀ haloalkoxy; and —N(R^{g})(R^{h}). Each occurrence of R^{g} and R^{h} is independently selected from the group consisting of H; C₁₋₄ alkyl; —C(═O)(C₂₋₂₀ alkyl); —C(═O)O(C₂₋₂₀ alkyl); and —S(O)₁₋₂(C₁₋₆ alkyl).

In certain embodiments, the modifying group (e.g., R*) is a C₂₋₃₀ (e.g., C₂₋₂₀, C₂₋₁₀, C₂₋₆) acyl group that is substituted with 1-2 (e.g., 2) independently selected R^{f}. In certain embodiments, each occurrence of R^{f} can independently selected from the group consisting of —C(═O)(OH) and —N(R^{g})(R^{h}); e.g., in which one of R^{g} and R^{h} is independently selected from the group consisting of —C(═O)(C₂₋₂₀ alkyl); —C(═O)O(C₂₋₂₀ alkyl); and —S(O)₁₋₂(C₁₋₆ alkyl), e.g., —C(═O)(C₂₋₂₀ alkyl). By way of example, the modifying group (e.g., R*) can be:

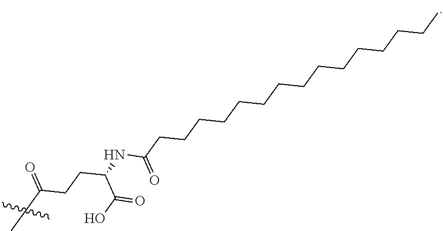 [SMILES: *C(=O)CC[C@H](NC(=O)CCCCCCCCCCCCCCC)C(=O)O]

As an additional example, R* can be:

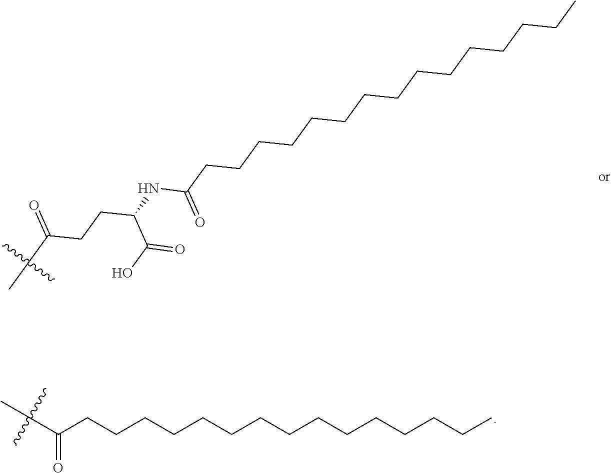 [SMILES: *C(=O)CC[C@H](NC(=O)CCCCCCCCCCCCCCC)C(=O)O ; *C(=O)CCCCCCCCCCCCCCC]

In some embodiments, R⁵ is a a C-terminal amino acid that is optionally substituted with from 1-2 modifying groups (e.g., 1-2 groups selected from an acyl group and a PEG group).

In certain embodiments, R⁵ is a C-terminal lysine that is optionally substituted with from 1-2 modifying groups (e.g., 1-2 groups selected from an acyl group and a PEG group). For example, R⁵ can be a C-terminal L-lysine that is optionally substituted with from 1-2 modifying groups (e.g., 1-2 groups selected from an acyl group and a PEG group).

In certain embodiments, R⁵ has formula (XI-OH):

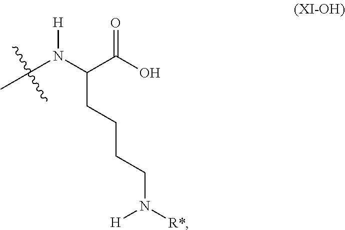

wherein R* is H or a modifying group (e.g., an acyl group and a PEG group). In certain of these embodiments, formula (XI-OH) is L. In other embodiments, formula (XI-OH) is D.

In certain embodiments of formula (XI) and (XI-OH), R* is H.

In certain embodiments, the modifying group (e.g., R*) is an acyl group. For example, the acyl group can be a C₂₋₃₀ (e.g., C₂₋₂₀, C₂₋₁₀, C₂₋₆) acyl group that is optionally substituted with from 1-2 independently selected R^{f}. Each occurrence of R^{f} is selected from the group consisting of —C(═O)(OH); —C(═O)(C₂₋₂₀ alkyl); —C(═O)O(C₂₋₂₀ alkyl); and —S(O)₁₋₂(C₁₋₆ alkyl); oxo; F; C₁₋₁₀ alkoxy; C₁₋₁₀ haloalkoxy; and —N(R^{g})(R^{h}). Each occurrence of R^{g} and R^{h} is independently selected from the group consisting of H; C₁₋₄ alkyl; —C(═O)(C₂₋₂₀ alkyl); —C(═O)O(C₂₋₂₀ alkyl); and —S(O)₁₋₂(C₁₋₆ alkyl).

In certain embodiments, the modifying group (e.g., R*) is a C₂₋₃₀ (e.g., C₂₋₂₀, C₂₋₁₀, C₂₋₆) acyl group that is substituted with 1-2 (e.g., 2) independently selected R^{f}. In certain embodiments, each occurrence of R^{f} can independently selected from the group consisting of —C(═O)(OH) and —N(R^{g})(R^{h}); e.g., in which one of R^{g} and R^{h} is independently selected from the group consisting of —C(═O)(C₂₋₂₀ alkyl); —C(═O)O(C₂₋₂₀ alkyl); and —S(O)₁₋₂(C₁₋₆ alkyl), e.g., —C(═O)(C₂₋₂₀ alkyl). By way of example, the modifying group (e.g., R*) can be:

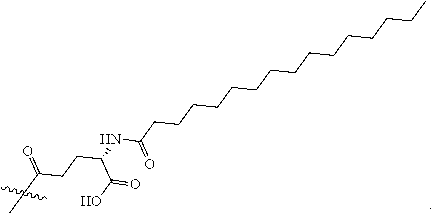 [SMILES: *C(=O)CC[C@H](NC(=O)CCCCCCCCCCCCCCC)C(=O)O]

As an additional example, R* can be:

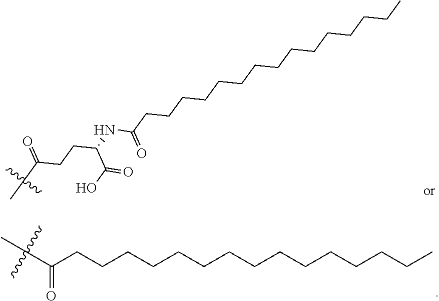 [SMILES: *C(=O)CC[C@H](NC(=O)CCCCCCCCCCCCCCC)C(=O)O ; *C(=O)CCCCCCCCCCCCCCC]

Other non-limiting, representative examples of acyl modifying groups are delineated, e.g., in Table 2.

In other embodiments, the modifying group is PEG. As used herein the general term “polyethylene glycol” or “PEG”, refers to mixtures of condensation polymers of ethylene oxide and water, in a branched or straight chain, represented by the general formula H (OCH₂CH₂)ₙOH, wherein n is at least 9. Absent any further characterization, the term is intended to include polymers of ethylene glycol with an average total molecular weight selected from the range of 5,000 to 40,000 Daltons. “Polyethylene glycol” or “PEG” is used in combination with a numeric suffix to indicate the approximate average molecular weight thereof. For example, PEG-5,000 refers to polyethylene glycol having a total molecular weight average of about 5,000. As used herein the term “PEGylated” or like terms refers to a compound that has been modified from its native state by linking a PEG chain to the peptide. A “PEGylated peptide” is a peptide that has a PEG chain covalently attached to the peptide itself.

In certain embodiments, R⁵ is a C-terminal amino acid selected from serine, glycine, and arginine.

In certain embodiments, R⁵ is a C-terminal amino acid amide selected from serinyl amide, glycyl amide, and argininyl amide.

In certain embodiments, R⁵ is a C-terminal glycyl residue (e.g., C-terminal glycine or C-terminal glycyl amide).

In certain embodiments, R⁵ is a C-terminal argininyl residue (e.g., C-terminal arginine or C-terminal argininyl amide).

In certain embodiments, R⁵ is a C-terminal serinyl residue (e.g., C-terminal serine or C-terminal serinyl amide).

### Non-Limiting Examples of W

In some embodiments, the amino acid sequence present in W is, or includes, the sequence that is present in native GLP-1-OH or GLP-1-NH₂. In other embodiments, the amino acid sequence present in W is, or includes, the sequence that is present in native GIP. In still other embodiments, the amino acid sequence present in Wis, or includes, a hybrid sequence having one or more fragments (e.g., functional fragments) present in native GLP-1-OH or GLP-1-NH₂ and one or more fragments (e.g., functional fragments) present in native GIP. This disclosure further contempates variations of the foregoing embodiments, e.g., W is, or includes, a conservatively substituted variation of the foregoing, meaning that one or more amino acid residues of an original peptide are replaced by different residues, and that the conservatively substituted peptide retains a desired biological activity. Examples of conservative substitutions include substitution of amino acids that tend not alter the secondary and/or tertiary structure of the compounds described herein, substitutions that do not change the overall or local hydrophobic character, substitutions that do not change the overall or local charge, substitutions by residues of equivalent side chain size, or substitutions by side chains with similar reactive groups.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar side chain volume are also within the scope of this disclosure.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

In certain embodiments, conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu. In general, conservative substitutions encompass residue exchanges with those of similar physicochemical properties (i.e. substitution of a hydrophobic residue for another hydrophobic amino acid).

In still other embodiments, the amino acid sequence present in W is, or includes, or is based upon a sequence that is present in a peptide having at least 0.01% of the GLP-1 receptor activation activity of the native GLP-1, such as at least 0.1%, 0.2%, 0.5%, 0.8%, 1%, 3%, 5%, 10%, 20%, 30%, 40%, 50%, 60%, 70%, 80%, 90%, 95%, 99% of the GLP-1 receptor activation activity of the native GLP-1-OH or GLP-1-NH₂ and/or at least 0.01% of the GIP receptor activation activity of GIP, such as at least 0.1%, 0.2%, 0.5%, 0.8%, 1%, 3%, 5%, 10%, 20%, 30%, 40%, 50%, 60%, 70%, 80%, 90%, 95%, 99% of the GIP receptor activation activity of the native GIP.

As used herein the term “native GLP-1” refers to a peptide comprising the sequence of human GLP-1(7-36, or 7-37), and term “native GIP” refers to a peptide comprising the sequence of human GIP (1-42). As used herein, a general reference to “GLP-1” or “GIP” in the absence of any further designation is intended to mean native GLP-1 or native GIP, respectively.

In some embodiments, W has formula (X): -GTF—W″—R⁵, wherein W″ is a sequence of 30-40 (e.g., 31-36, 33) amino acids.

In certain embodiments, W has formula (XII):

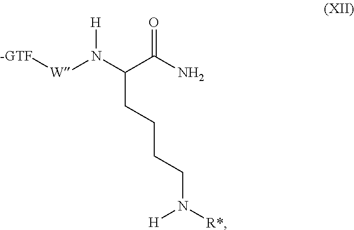

wherein W″ is a sequence of 30-40 (e.g., 31-35, e.g., 33) amino acids, and R* is H or a modifying group (e.g., an acyl group and a PEG group as described herein).

In certain embodiments, W has formula (XII-OH):

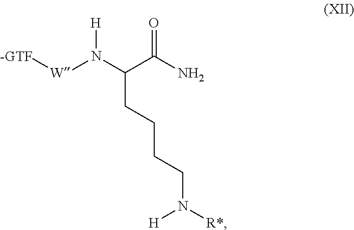

wherein W″ is a sequence of 30-40 (e.g., 31-35, e.g., 33) amino acids, and R* is H or a modifying group (e.g., an acyl group and a PEG group as described herein).

In some embodiments, W has formula (XIII):
-GTF—W′″-GPSSGAPPPS—R⁵ (SEQ ID NO: 1); wherein W′″ is a sequence of 20-30 (e.g., 21-25, e.g., 23) amino acids.

In some embodiments, W has formula (XIV-AA):
GT(Xaa3)(Xaa4)SD(Xaa7)S(Xaa9)(Xaa10)(Xaa11)(Xaa12)(Xaa13)(Xaa14)(Xaa15)(Xaa16)(Xaa17)(Xaa18)(Xaa19)(Xaa20)(Xaa21)WL(Xaa24)(Xaa25)(Xaa26)GP SSGAPPP(Xaa36)-R⁵ (SEQ ID NO: 35);
wherein:
Xaa3 is F;
Xaa4 is T or I (e.g., T);
Xaa7 is Y, V, L, or K* (e.g., Y);
Xaa9 is I or S (e.g., I);
Xaa10 is Y, Y*, Q, A, or (Aib) (e.g., Y);
Xaa11 is L, M, or L* (e.g., L);
Xaa12 is D or E (e.g., D);
Xaa13 is K, G, R, or E (e.g., K);
Xaa14 is Q or I (e.g., Q);
Xaa15 is A, H, or R (e.g., A);
Xaa16 is A, Q, or V (e.g., A);
Xaa17 is A, (Aib), K*, K, or Q (e.g., (Aib));
Xaa18 is A, D, E, (Aib), or L (e.g., A, D, E, or L (e.g., E));
Xaa19 is F, or A (e.g., F);
Xaa20 is V or I (e.g., V);
Xaa21 is N, A, Q, K*, or E (e.g., N);
Xaa24 is I, L or V (e.g., L);
Xaa25 is A, K, or I (e.g., A);
Xaa26 is Q-R, G-R-G-K*, Q, or G (e.g., G); and
Xaa36 is S or absent (e.g., S).

In some embodiments (e.g., when W has formula (XIV-AA)), W has formula (XIV):
GTF (Xaa4)SD(Xaa7)S(Xaa9)(Xaa10)(Xaa11)(Xaa12)(Xaa13)QA(Xaa16)(Xaa 17)(Xaa18)F-(Xaa20)(Xaa21)WL(Xaa24)(Xaa25)GGPSSGAPPPS-R⁵ (SEQ ID NO:2);
wherein:
Xaa4 is T or I (e.g., T);
Xaa7 is Y, V, or L (e.g., Y);
Xaa9 is I or S (e.g., I);
Xaa10 is Y, Q, or A (e.g., Y);
Xaa11 is L, M, or L* (e.g., L);
Xaa12 is D or E (e.g., D);
Xaa13 is K, G, or E (e.g., K);
Xaa16 is A or V (e.g., A);
Xaa17 is (Aib) or K (e.g., (Aib));
Xaa18 is E or L (e.g., E);
Xaa20 is V or I (e.g., V);
Xaa21 is N, A, or E (e.g., N);
Xaa24 is L or V (e.g., L); and
Xaa25 is A or K (e.g., A).

In certain embodiments, W has formula (XIV-A):
GTF(Xaa4)SD(Xaa7)S(Xaa9)(Xaa10)(Xaa11)(Xaa12)(Xaa13)QA(Xaa16)(Aib)-(Xaa18)F(Xaa20)(Xaa21)WL(Xaa24)(Xaa25)GGPSSGAPPPS-R⁵ (SEQ ID NO: 3), wherein each of the “Xaa” variables is as defined in conjunction with Formula (XIV). In certain of these embodiments, R⁵ can be as defined anywhere herein (e.g., can have formula (XI); e.g., D or L formula (XI), and R* can be as defined anywhere herein).

In certain embodiments, W has formula (XIV-B):
GTFTSDYSIYLDKQAA(Aib)EFVNWLLAGGPSSGAPPPS-R⁵ (SEQ ID NO: 4). In certain of these embodiments, R⁵ can be as defined anywhere herein (e.g., can have formula (XI); e.g., D or L formula (XI), and R* can be as defined anywhere herein).

As used herein “(Aib)” refers to 2-aminoisobutyric acid (also known as a-aminoisobutyric acid or α-methylalanine or 2-methylalanine).

As used herein, Y* refers to 2-amino-3-(4-hydroxyphenyl)-2-methylpropanoic acid (e.g., (S)-2-amino-3-(4-hydroxyphenyl)-2-methylpropanoic acid). As used herein, L* refers to 2-amino-2-methylpentanoic acid (e.g., (S)-2-amino-2-methylpentanoic acid).
or a C-terminal amino acid or an amino acid ester or an amino acid amide thereof.

As used herein K* is a lysine residue substituted with a modifying group, or a C-terminal amino acid or an amino acid ester or amino acid amide thereof.

Non-limiting examples of K* can include an amino acid residue selected from:

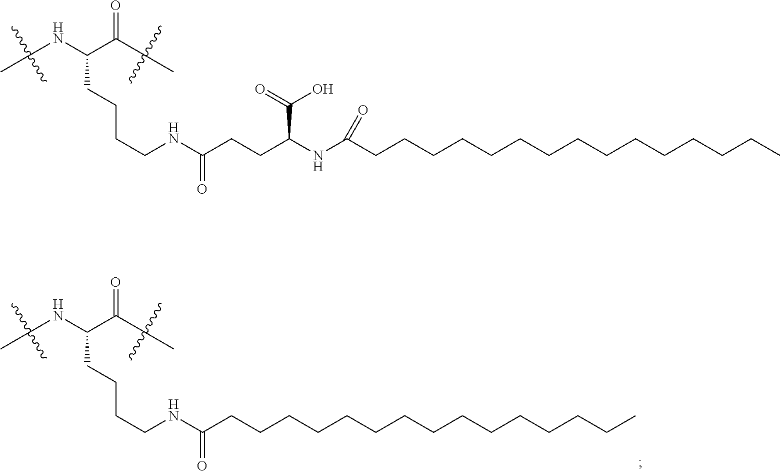 [SMILES: *N[C@@H](CCCCNC(=O)CC[C@H](NC(=O)CCCCCCCCCCCCCCC)C(=O)O)C(*)=O ; *N[C@@H](CCCCNC(=O)CCCCCCCCCCCCCCC)C(*)=O]

or a C-terminal amino acid or an amino acid ester or an amino acid amide thereof.

In some embodiments, W is represented by formula (X), (XII), (XIII), (XIV-AA), (XIV), (XIV-A), (XIV-B), (XV), or any of SEQ ID 1-34.

For example, W can have any of the sequences delineated in Table 2.

TABLE 2

| SEQ | |
|---|---|
| ID NO: | Sequence |
| 5 | GTFTSDYSIYLDKQAA(Aib)EFVNWLLAGGPSSGAPPPSK-NH₂ |
| 6 | GTFTSDYSIYLDKQAA(Aib)EFVNWLLAGGPSSGAPPPSK* |
| | K* = |
| | 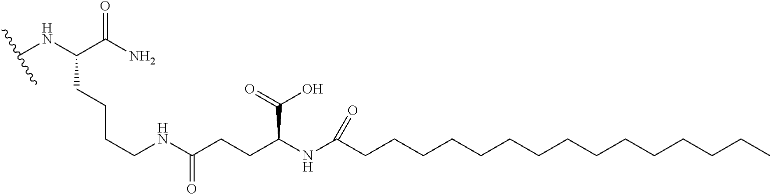 [SMILES: *N[C@@H](CCCCNC(=O)CC[C@H](NC(=O)CCCCCCCCCCCCCCC)C(=O)O)C(N)=O] |
| 7 | GTFTSDYSIYLDKQAA(Aib)EFVNWLLAGGPSSGAPPPSK* |
| | K* = |
| | 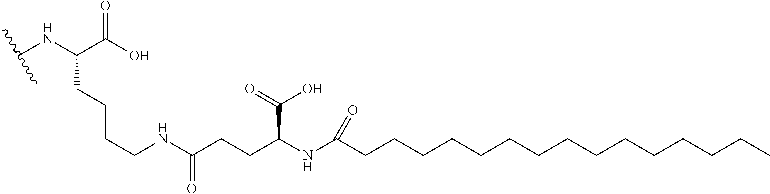 [SMILES: *N[C@@H](CCCCNC(=O)CC[C@H](NC(=O)CCCCCCCCCCCCCCC)C(=O)O)C(=O)O] |
| 8 | GTFTSDYSIYLDKQAA(Aib)EFVNWLLAGGPSSGAPPPSK* |
| | K* = |
| | 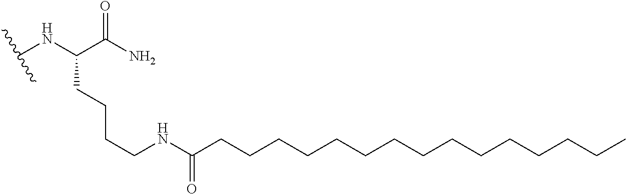 [SMILES: *N[C@@H](CCCCNC(=O)CCCCCCCCCCCCCCC)C(N)=O] |
| 9 | GTFTSDYSIYLDKQAA(Aib)EFVNWLLAGGPSSGAPPPSK*, |
| | K* = |
| | 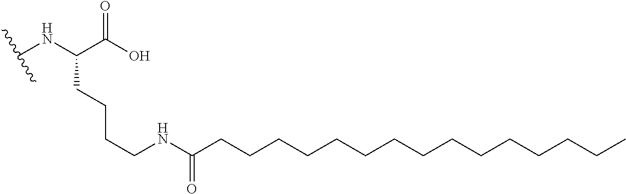 [SMILES: *N[C@@H](CCCCNC(=O)CCCCCCCCCCCCCCC)C(=O)O] |
| 10 | GTFTSD [symbol] S [symbol] YL [symbol] QAA [symbol] EF [symbol] WL [symbol] GGPSSGAPPPSK*-NH₂, |
| | K* = |
| | 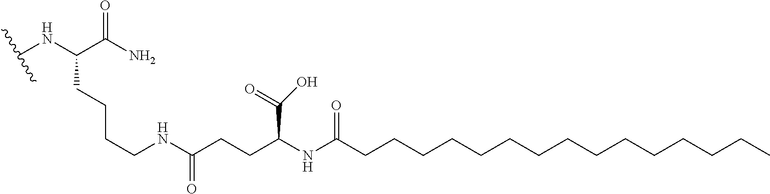 [SMILES: *N[C@@H](CCCCNC(=O)CC[C@H](NC(=O)CCCCCCCCCCCCCCC)C(=O)O)C(N)=O] |
| 11 | GTF [symbol] SDYS [symbol] DK [symbol] FVNWLLA [symbol] GPSSGAPPPSK*-NH₂, |
| | K* = |
| | 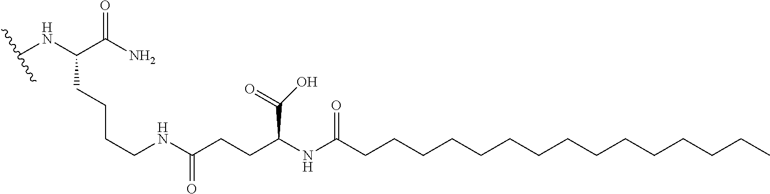 [SMILES: *N[C@@H](CCCCNC(=O)CC[C@H](NC(=O)CCCCCCCCCCCCCCC)C(=O)O)C(N)=O] |
| 12 | GTFTSD [symbol] SIYLDKQAA(Aib)EFVNWLLAGGPSSGAPPPSK-NH₂ |
| 13 | GTFTSDYSIYLD [symbol] QAA(Aib)EFVNWLLAGGPSSGAPPPSK-NH₂ |
| 14 | GTFTSDYSIYLDKQA [symbol] (Aib)EFVNWLLAGGPSSGAPPPSK-NH₂ |
| 15 | GTFTSDYSIYLDKQAA(Aib) [symbol] FVNWLLAGGPSSGAPPPSK-NH₂ |
| 16 | GTFTSDYSIYLDKQAA(Aib)EF [symbol] NWLLAGGPSSGAPPPSK-NH₂ |
| 17 | GTFTSDYSIYLDKQAA(Aib)EFV [symbol] WLLAGGPSSGAPPPSK-NH₂ |
| 18 | GTFTSDYSIYLDKQA [symbol] (Aib)EF [symbol] NWLLAGGPSSGAPPPSK-NH₂ |
| 19 | GTFTSDYSI [symbol] DKQAA(Aib)EFVNWLLAGGPSSGAPPPSK*-NH₂ |
| | K* = |
| | 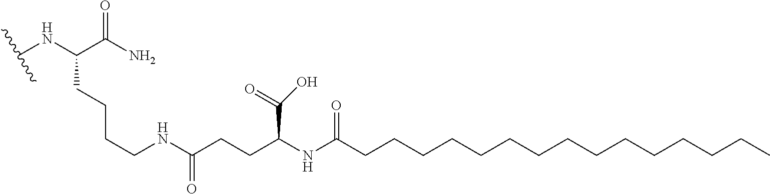 [SMILES: *N[C@@H](CCCCNC(=O)CC[C@H](NC(=O)CCCCCCCCCCCCCCC)C(=O)O)C(N)=O] |
| 20 | GTFTSDYSI [symbol] DKQAA(Aib)EFVNWLLAGGPSSGAPPPSK*-NH₂ |
| | K* = |
| | 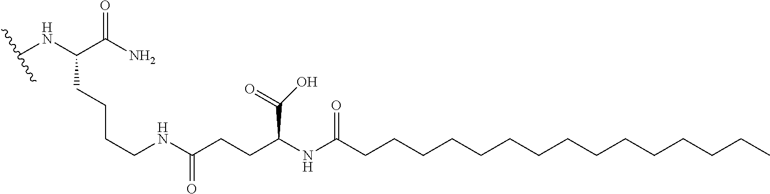 [SMILES: *N[C@@H](CCCCNC(=O)CC[C@H](NC(=O)CCCCCCCCCCCCCCC)C(=O)O)C(N)=O] |
| 21 | GTFTSDYSIY [symbol] DKQAA(Aib)EFVNWLLAGGPSSGAPPPSK*-NH₂ |
| | K* = |
| | 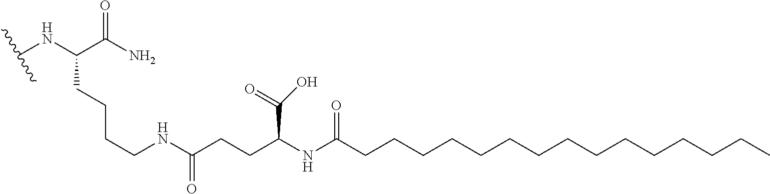 [SMILES: *N[C@@H](CCCCNC(=O)CC[C@H](NC(=O)CCCCCCCCCCCCCCC)C(=O)O)C(N)=O] |
| | L* = |
| | 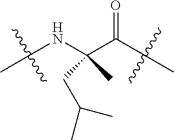 [SMILES: *N[C@@](C)(CC(C)C)C(*)=O] |
| 22 | GTFTSDYSIYLD [symbol] QAA(Aib)EFVNWLLAGGPSSGAPPPS-NH₂ |
| 23 | 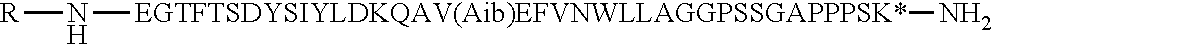 |
| | [symbol] |
| | 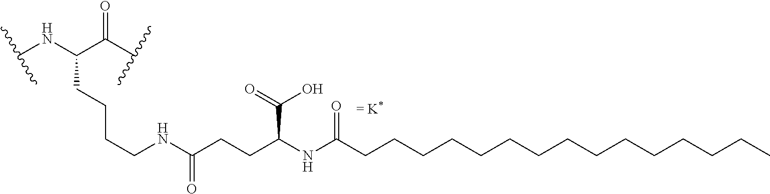 [SMILES: *N[C@@H](CCCCNC(=O)CC[C@H](NC(=O)CCCCCCCCCCCCCCC)C(=O)O)C(*)=O] |
| 24 | 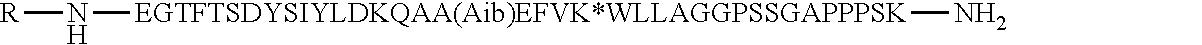 |
| | [symbol] |
| | 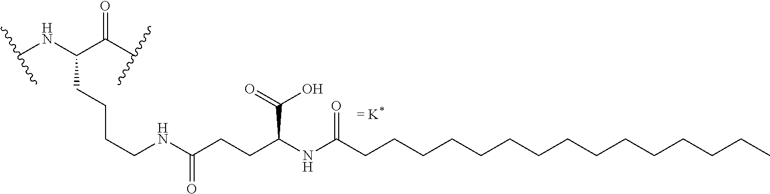 [SMILES: *N[C@@H](CCCCNC(=O)CC[C@H](NC(=O)CCCCCCCCCCCCCCC)C(=O)O)C(*)=O] |
| 25 | 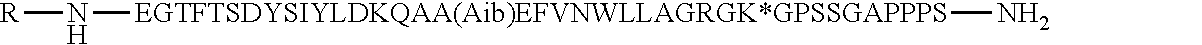 |
| | [symbol] |
| | 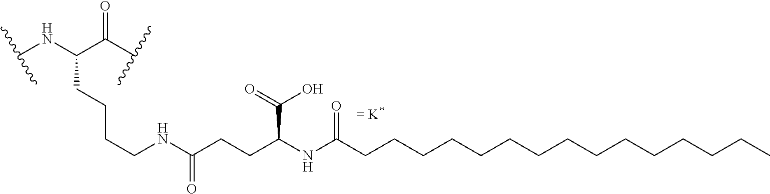 [SMILES: *N[C@@H](CCCCNC(=O)CC[C@H](NC(=O)CCCCCCCCCCCCCCC)C(=O)O)C(*)=O] |
| 26 | 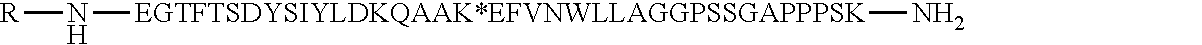 |
| | [symbol] |
| | 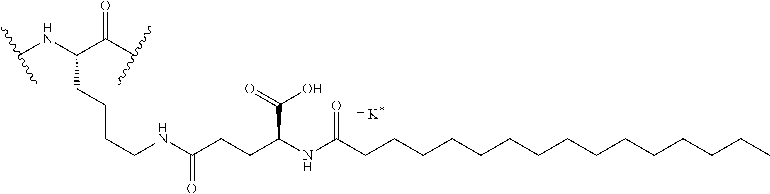 [SMILES: *N[C@@H](CCCCNC(=O)CC[C@H](NC(=O)CCCCCCCCCCCCCCC)C(=O)O)C(*)=O] |
| 27 | 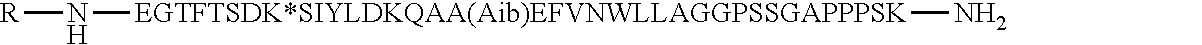 |
| | [symbol] |
| | 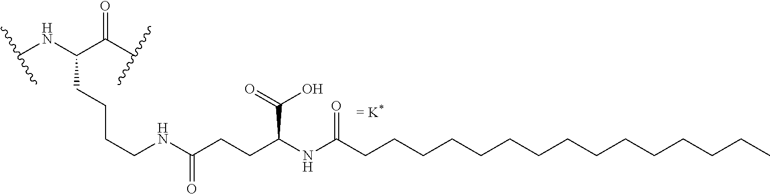 [SMILES: *N[C@@H](CCCCNC(=O)CC[C@H](NC(=O)CCCCCCCCCCCCCCC)C(=O)O)C(*)=O] |
| 28 | GTFTSDYSIYLDKQAA(Aib)EFVNWLLAGGPSSGAPPPSK*-NH₂ |
| | 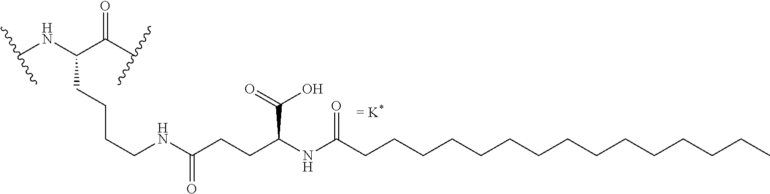 [SMILES: *N[C@@H](CCCCNC(=O)CC[C@H](NC(=O)CCCCCCCCCCCCCCC)C(=O)O)C(*)=O] |
| 29 | GTFTSDYSI [symbol] LDKQAA(Aib)EFVNWLLAGGPSSGAPPPSK*-NH₂ |
| | 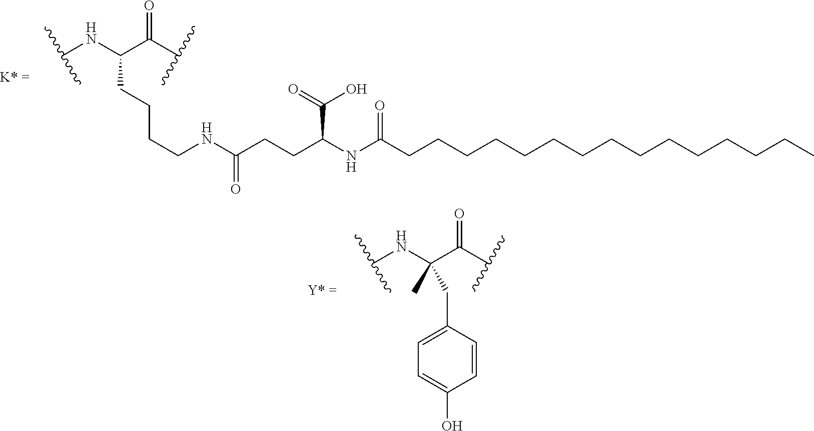 [SMILES: *N[C@@H](CCCCNC(=O)CC[C@H](NC(=O)CCCCCCCCCCCCCCC)C(=O)O)C(*)=O ; *N[C@@](C)(Cc1ccc(O)cc1)C(*)=O] |
| 30 | GTFTSDYSIYLDKQA [symbol] (Aib)EFVNWLLAGGPSSGAPPPSK*-NH₂ |
| | 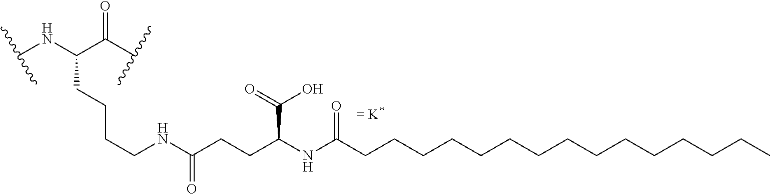 [SMILES: *N[C@@H](CCCCNC(=O)CC[C@H](NC(=O)CCCCCCCCCCCCCCC)C(=O)O)C(*)=O] |
| 31 | GTFTSDYSIYLDKQAA(Aib)EFV [symbol] WLLAGGPSSGAPPPSK-NH₂ |
| | 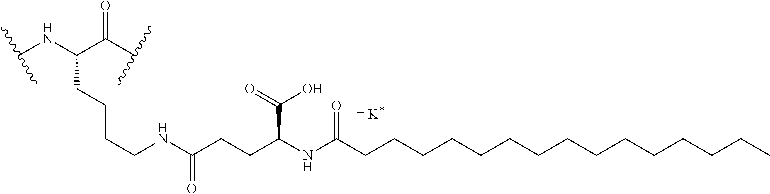 [SMILES: *N[C@@H](CCCCNC(=O)CC[C@H](NC(=O)CCCCCCCCCCCCCCC)C(=O)O)C(*)=O] |
| 32 | GTFTSDYSIYLDKQAA(Aib)EFVNWLLAG [symbol] GPSSGAPPPS-NH₂ |
| | 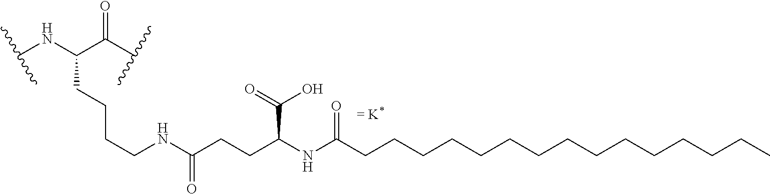 [SMILES: *N[C@@H](CCCCNC(=O)CC[C@H](NC(=O)CCCCCCCCCCCCCCC)C(=O)O)C(*)=O] |
| 33 | GTFTSDYSIYLDKQAA [symbol] EFVNWLLAGGPSSGAPPPSK-NH₂ |
| | 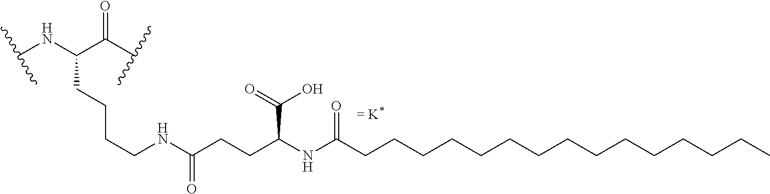 [SMILES: *N[C@@H](CCCCNC(=O)CC[C@H](NC(=O)CCCCCCCCCCCCCCC)C(=O)O)C(*)=O] |
| 34 | GTFTSD [symbol] SIYLDKQAA(Aib)EFVNWLLAGGPSSGAPPPSK-NH₂ |
| | 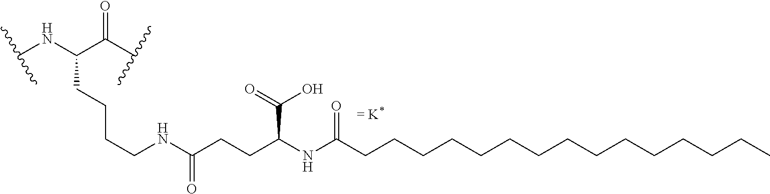 [SMILES: *N[C@@H](CCCCNC(=O)CC[C@H](NC(=O)CCCCCCCCCCCCCCC)C(=O)O)C(*)=O] |

In some embodiments, W is represented by any one of SEQ ID 1-34 (e.g., SEQ. ID 5-27; e.g., SEQ ID 5-29; (e.g., SEQ ID 6); e.g., SEQ ID 28-29; e.g., SEQ ID 30-34).

In some embodiments, W is represented by any one of SEQ ID 1-27 (e.g., SEQ. ID 5-27; e.g., SEQ ID 6).

In some embodiments, W is represented by any one of SEQ ID 28-29 (e.g., 28, 29).

In certain embodiments, W has the sequence of SEQ ID 6.

### Non-Limiting Combinations of Formulae (IAA), (IA), and (I)

[1] In some embodiments, the compound has formula (I-A), (I-AA, or (I-AB), ring A is a saturated or unsaturated monocyclic ring that includes from 3-8 (e.g., 4-8, 4-7, 5-6) ring atoms, and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH.
[1A] In some embodiments, the compound has formula (IA-A), (IA-AA), or (IA-AB), ring A is a saturated or unsaturated monocyclic ring that includes from 3-8 (e.g., 4-8, 4-7, 5-6) ring atoms, and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH. In certain embodiments, a′ is 1; each of R^{3a} and R^{3b} is H.
[2] In some embodiments, the compound has formula (I-A), (I-AA), or (I-AB), (I-A), (I-AA), or (I-AB), ring A is a saturated monocyclic ring that includes from 3-8 (e.g, 4-8, 4-7, 5-6) ring atoms, and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH.
[2A] In some embodiments, the compound has formula (IA-A), (IA-AA), or (IA-AB), (IA-B), (IA-BA, or (IA-BB), ring A is a saturated monocyclic ring that includes from 3-8 (e.g., 4-8, 4-7, 5-6) ring atoms, and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH. In certain embodiments, a′ is 1; each of R^{3a} and R^{3b} is H.
[3] In some embodiments, the compound has formula (I-A), (I-AA), or (I-AB), ring A is a saturated, monocyclic, 6-membered ring having formula (III) (i.e., A₁ is not a bond), and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH.
[3A] In some embodiments, the compound has formula (IA-A), (IA-AA), or (IA-AB), ring A is a saturated, monocyclic, 6-membered ring having formula (III) (i.e., A₁ is not a bond), and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH. In certain embodiments, a′ is 1; each of R^{3a} and R^{3b} is H.
[4] In some embodiments, the compound has formula (I-A), (I-AA), or (I-AB), ring A is a saturated, monocyclic, 5-membered ring having formula (III-A) (i.e., A₁ in formula (III) is a bond), and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH.
[4A] In some embodiments, the compound has formula (IA-A), (IA-AA), or (IA-AB), ring A is a saturated, monocyclic, 5-membered ring having formula (III-A) (i.e., A₁ in formula (III) is a bond), and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH. In certain embodiments, a′ is 1; each of R^{3a} and R^{3b} is H.
[5] In some embodiments, the compound has formula (I-A), (I-AA), or (I-AB), ring A is a saturated or unsaturated bicyclic or tricyclic ring that includes from 6-14 (e.g, 8-10) ring atoms, and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH.
[5A] In some embodiments, the compound has formula (IA-A), (IA-AA), or (IA-AB), ring A is a saturated or unsaturated bicyclic or tricyclic ring that includes from 6-14 (e.g., 8-10) ring atoms, and L has formula (VIII): —(CH₂)ₘ—X¹—(CH₂)ₙ—X²—(CH₂)ₚ—. In certain embodiments, one, two, or three of R¹, R², and R⁴ are H. In certain embodiments, R³ is —C(O)OH. In certain embodiments, a is 0, 1, or 2 (e.g., 1). In certain embodiments, each of R¹, R², and R⁴ is H, a is 0, 1, or 2 (e.g., 1), and R³ is —C(O)OH. In certain embodiments, a′ is 1; each of R^{3a} and R^{3b} is H.
[6] In some embodiments, the compound has formula (I-A), (I-AA), or (I-AB), ring A is a saturated or unsaturated monocyclic ring that includes from 3-8 (e.g., 4-8, 4-7, 5-6) ring atoms, L has formula (VIII): —(CH₂)ₘ—X¹—(CH₂)ₙ—X²—(CH₂)ₚ—, and W is represented by formula (X), (XII), (XIII), (XIV), (XIV-A), (XIV-B), or any of SEQ ID 1-27 (e.g., SEQ ID 5-27, e.g., SEQ ID 6). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).
[6A] In some embodiments, the compound has formula (IA-A), (IA-AA), or (IA-AB), ring A is a saturated or unsaturated monocyclic ring that includes from 3-8 (e.g., 4-8, 4-7, 5-6) ring atoms, L has formula (VIII): —(CH₂)ₘ—X¹—(CH₂)ₙ—X²—(CH₂)ₚ—, and W is represented by formula (X), (XII), (XIII), (XIV-AA), (XIV), (XIV-A), (XIV-B), (XV), or any of SEQ ID 1-34 (e.g., SEQ ID 5-27; e.g., SEQ ID 5-29; (e.g., SEQ ID 6); e.g., SEQ ID 28-29; e.g., SEQ ID 30-34). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.
[7] In some embodiments, the compound has formula (I-A), (I-AA), or (I-AB), ring A is a saturated monocyclic ring that includes from 3-8 (e.g., 4-8, 4-7, 5-6) ring atoms, L has formula (VIII): —(CH₂)ₘ—X¹—(CH₂)ₙ—X²—(CH₂)ₚ—, and W is represented by (X), (XII), (XIII), (XIV), (XIV-A), (XIV-B), or any of SEQ ID 1-27 (e.g., SEQ ID 5-27, e.g., SEQ ID 6). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).
[7A] In some embodiments, the compound has formula (I-A), (I-AA), or (I-AB), ring A is a saturated monocyclic ring that includes from 3-8 (e.g., 4-8, 4-7, 5-6) ring atoms, L has formula (VIII): —(CH₂)ₘ—X¹—(CH₂)ₙ—X²—(CH₂)ₚ—, and W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.
[8] In some embodiments, the compound has formula (I-A), (I-AA), or (I-AB), ring A is a saturated, monocyclic, 6-membered ring having formula (III) (i.e., A₁ is not a bond), L has formula (VIII): —(CH₂)ₘ—X¹—(CH₂)ₙ—X²—(CH₂)ₚ—, and W is represented by formula (X), (XII), (XIII), (XIV), (XIV-A), (XIV-B), or any of SEQ ID 1-27 (e.g., SEQ ID 5-27, e.g., SEQ ID 6). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).
[8A] In some embodiments, the compound has formula (IA-A), (IA-AA), or (IA-AB), ring A is a saturated, monocyclic, 6-membered ring having formula (III) (i.e., A₁ is not a bond), L has formula (VIII): —(CH₂)ₘ—X¹—(CH₂)ₙ—X²—(CH₂)ₚ—, and W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.
[9] In some embodiments, the compound has formula (I-A), (I-AA), or (I-AB), ring A is a saturated, monocyclic, 5-membered ring having formula (III-A) (i.e., A₁ in formula (III) is a bond), L has formula (VIII): —(CH₂)ₘ—X¹—(CH₂)ₙ—X²—(CH₂)ₚ—, and W is represented by formula (X), (XII), (XIII), (XIV), (XIV-A), (XIV-B), or any of SEQ ID 1-27 (e.g., SEQ ID 5-27, e.g., SEQ ID 6). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).
[9A] In some embodiments, the compound has formula (IA-A), (IA-AA), or (IA-AB), ring A is a saturated, monocyclic, 5-membered ring having formula (III-A) (i.e., A₁ in formula (III) is a bond), L has formula (VIII): —(CH₂)ₘ—X¹—(CH₂)ₙ—X²—(CH₂)ₚ—, and W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.
[10] In some embodiments, the compound has formula (I-A), (I-AA), or (I-AB), ring A is a saturated or unsaturated bicyclic or tricyclic ring that includes from 6-14 (e.g, 8-10) ring atoms, L has formula (VIII): —(CH₂)ₘ—X¹—(CH₂)ₙ—X²—(CH₂)ₚ—, and W is represented by formula (X), (XII), (XIII), (XIV), (XIV-A), (XIV-B), or any of SEQ ID 1-27 (e.g., SEQ ID 5-27, e.g., SEQ ID 6). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).
[10A] In some embodiments, the compound has formula (IA-A), (IA-AA), or (IA-AB), ring A is a saturated or unsaturated bicyclic or tricyclic ring that includes from 6-14 (e.g., 8-10) ring atoms, L has formula (VIII): —(CH₂)ₘ—X¹—(CH₂)ₙ—X²—(CH₂)ₚ—, and W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34). In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.
[11] In some embodiments:
the compound has formula (I-A), (I-AA), or (I-AB);
ring A is a saturated monocyclic ring that includes from 3-8 (e.g., 4-8, 4-7, 5-6) ring atoms;
L has formula (VIII): —(CH₂)ₘ—X¹—(CH₂)ₙ—X²—(CH₂)ₚ—,
W is represented by formula (X), (XII), (XIII), (XIV), (XIV-A), (XIV-B), or any of SEQ ID 1-27 (e.g., SEQ ID 5-27, e.g., SEQ ID 6); and
m is 2-6 (e.g., 2), X¹ is —N(R′)C(O)— (e.g., —N(H)C(O)—), and n+p=2 (e.g., each of n and p is 1), and X² is —S—; or
m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl; or
m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃); or
m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.

In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).

In certain of these embodiments, m is 2-6 (e.g., 2), X¹ is —N(R′)C(O)— (e.g., —N(H)C(O)—), and n+p=2 (e.g., each of n and p is 1), and X² is —S—. For example, L can be —CH₂CH₂NHC(O)CH₂SCH₂—.

In certain of these embodiments, any one of SEQ ID 5-27 (e.g., SEQ ID 6) represents W.
[11A] In some embodiments:
the compound has formula (IA-A), (IA-AA), or (IA-AB);
ring A is a saturated monocyclic ring that includes from 3-8 (e.g., 4-8, 4-7, 5-6) ring atoms;
L has formula (VIII): —(CH₂)ₘ—X¹—(CH₂)ₙ—X²—(CH₂)ₚ—, and
W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34).

In certain embodiments, mis 2-6 (e.g., 2), X¹ is —N(R′)C(O)— (e.g., —N(H)C(O)—), and n+p=2 (e.g., each of n and p is 1), and X² is —S—; or
m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl; or
m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃); or
m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.

In certain embodiments, m is 2-6 (e.g., 2), X¹ is —N(R′)C(O)— (e.g., —N(H)C(O)—), n+p=0, and X² is: heterocycloalkylene including from 4-6 (e.g., 4) ring atoms, wherein from 1-3 (e.g., 1) ring atoms are each independently selected from the group consisting of N(R^{a}), O, and S; and which is optionally substituted with from 1-5 independently selected R^{d} (e.g., X² can be

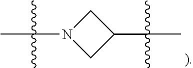 [SMILES: *C1CN(*)C1]

In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.

In certain of these embodiments, m is 2-6 (e.g., 2), X¹ is —N(R′)C(O)— (e.g., —N(H)C(O)—), and n+p=2 (e.g., each of n and p is 1), and X² is —S—. For example, L can be —CH₂CH₂NHC(O)CH₂SCH₂—.

In certain of these embodiments, any one of SEQ ID 5-34 (e.g., SEQ ID 6) represents W.
[12] In some embodiments:
the compound has formula (I-A), (I-AA), or (I-AB);
ring A is a saturated, monocyclic, 6-membered ring having formula (III) (i.e., A₁ is not a bond);
L has formula (VIII): —(CH₂)ₘ—X¹—(CH₂)ₙ—X²—(CH₂)ₚ—;
W is represented by formula (X), (XII), (XIII), (XIV), (XIV-A), (XIV-B), or any of SEQ ID 1-27 (e.g., SEQ ID 5-27, e.g., SEQ ID 6); and
m is 2-6 (e.g., 2), X¹ is —N(R′)C(O)— (e.g., —N(H)C(O)—), and n+p=2 (e.g., each of n and p is 1), and X² is —S—; or
m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl; or
m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃); or
m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.

In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).

In certain of these embodiments, m is 2-6 (e.g., 2), X¹ is —N(R′)C(O)— (e.g., —N(H)C(O)—), and n+p=2 (e.g., each of n and p is 1), and X² is —S—. For example, L can be —CH₂CH₂NHC(O)CH₂SCH₂—.

In certain of these embodiments, any one of SEQ ID 5-27 (e.g., SEQ ID 6) represents W.

In certain formula (III), 6-membered ring embodiments, A₁, A₂, A₃ and A₄ are each independently selected from the group consisting of CH₂, CHR^{b}, or C(R^{b})₂.

In certain formula (III), 6-membered ring embodiments, one or two (e.g., one) of A₁, A₂, A₃ and A₄ are independently selected from the group consisting of CHR^{b} and C(R^{b})₂, and the others are CH₂.

In certain formula (III), 6-membered ring embodiments, A₁, A₂, A₃ and A₄ are each CH₂.

In certain formula (III), 6-membered ring embodiments, A₁, A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₃ are both CH₂. In certain of these embodiments, A₁ is CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₁, A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₃ are both CH₂. In certain of these embodiments, A₁ is CH₂. In certain of these embodiments, A₄ is N(R^{a}) (e.g., A₄ can be NH). In other embodiments, A₄ is O.

In certain formula (III), 6-membered ring embodiments, A₁ is C(O); and A₂, A₃ and A₄ are each independently selected from the group consisting of CH₂, CHR^{b}, or C(R^{b})₂. In certain of these embodiments, one or two (e.g., one) of A₂, A₃ and A₄ are independently selected from the group consisting of CHR^{b} and C(R^{b})₂, and the other(s) is/are CH₂.

In certain formula (III), 6-membered ring embodiments, A₁ is C(O); and A₂, A₃ and A₄ are each CH₂.

In certain formula (III), 6-membered ring embodiments, A₁ is C(O); A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₄ are both CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₁ is C(O); A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₃ are independently selected from the group consisting of CHR^{b} and C(R^{b})₂, and the other (where applicable) is CH₂. In other embodiments, A₂ and A₃ are both CH₂. In certain of these embodiments, A₄ is N(R^{a}) (e.g., A₄ can be NH). In other embodiments, A₄ is O. In certain formula (III), 6-membered ring embodiments, A₁, A₂, A₃ and A₄ are each independently selected from the group consisting of CH₂, CHR^{b}, or C(R^{b})₂.
[12A] In some embodiments:
the compound has formula (IA-A), (IA-AA), or (IA-AB);
ring A is a saturated, monocyclic, 6-membered ring having formula (III) (i.e., A₁ is not a bond);
L has formula (VIII): —(CH₂)ₘ—X¹—(CH₂)ₙ—X²—(CH₂)ₚ—; and
W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34).

In certain embodiments, m is 2-6 (e.g., 2), X¹ is —N(R′)C(O)— (e.g., —N(H)C(O)—), and n+p=2 (e.g., each of n and p is 1), and X² is —S—; or
m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl; or
m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃); or
m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.

In certain embodiments, m is 2-6 (e.g., 2), X¹ is —N(R′)C(O)— (e.g., —N(H)C(O)—), n+p=0, and X² is: heterocycloalkylene including from 4-6 (e.g., 4) ring atoms, wherein from 1-3 (e.g., 1) ring atoms are each independently selected from the group consisting of N(R^{a}), O, and S; and which is optionally substituted with from 1-5 independently selected R^{d} (e.g., X² can be

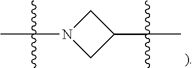 [SMILES: *C1CN(*)C1]

In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.

In certain of these embodiments, m is 2-6 (e.g., 2), X¹ is —N(R′)C(O)— (e.g., —N(H)C(O)—), and n+p=2 (e.g., each of n and p is 1), and X² is —S—. For example, L can be —CH₂CH₂NHC(O)CH₂SCH₂—.

In certain of these embodiments, any one of SEQ ID 5-34 (e.g., SEQ ID 6) represents W.

In certain formula (III), 6-membered ring embodiments, A₁, A₂, A₃ and A₄ are each independently selected from the group consisting of CH₂, CHR^{b}, or C(R^{b})₂.

In certain formula (III), 6-membered ring embodiments, one or two (e.g., one) of A₁, A₂, A₃ and A₄ are independently selected from the group consisting of CHR^{b} and C(R^{b})₂, and the others are CH₂.

In certain formula (III), 6-membered ring embodiments, A₁, A₂, A₃ and A₄ are each CH₂.

In certain formula (III), 6-membered ring embodiments, A₁, A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₃ are both CH₂. In certain of these embodiments, A₁ is CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₁, A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₃ are both CH₂. In certain of these embodiments, A₁ is CH₂. In certain of these embodiments, A₄ is N(R^{a}) (e.g., A₄ can be NH). In other embodiments, A₄ is O.

In certain formula (III), 6-membered ring embodiments, A₁ is C(O); and A₂, A₃ and A₄ are each independently selected from the group consisting of CH₂, CHR^{b}, or C(R^{b})₂. In certain of these embodiments, one or two (e.g., one) of A₂, A₃ and A₄ are independently selected from the group consisting of CHR^{b} and C(R^{b})₂, and the other(s) is/are CH₂.

In certain formula (III), 6-membered ring embodiments, A₁ is C(O); and A₂, A₃ and A₄ are each CH₂.

In certain formula (III), 6-membered ring embodiments, A₁ is C(O); A₂ and A₄ are each independently selected from the group consisting of CH₂, CHR^{b}, and C(R^{b})₂; and A₃ is O; S; SO₂; or N(R^{a}). In certain of these embodiments, one or two (e.g., one) of A₂ and A₄ are independently selected from the group consisting of CHR^{b} and C(R^{b})₂, and the other (where applicable) is CH₂. In other embodiments, A₂ and A₄ are both CH₂. In certain of these embodiments, A₃ is N(R^{a}) (e.g., A₃ can be NH). In other embodiments, A₃ is O. In still other embodiments, A₃ is SO₂.

In certain formula (III), 6-membered ring embodiments, A₁ is C(O); A₂ and A₃ are each independently selected from the group consisting of CH₂, CHR^{b}, and C(R^{b})₂; and A₄ is O; S; or N(R^{a}). In certain of these embodiments, one or two (e.g., one) of A₂ and A₃ are independently selected from the group consisting of CHR^{b} and C(R^{b})₂, and the other (where applicable) is CH₂. In other embodiments, A₂ and A₃ are both CH₂. In certain of these embodiments, A₄ is N(R^{a}) (e.g., A₄ can be NH). In other embodiments, A₄ is O. In certain formula (III), 6-membered ring embodiments, A₁, A₂, A₃ and A₄ are each independently selected from the group consisting of CH₂, CHR^{b}, or C(R^{b})₂.
[13] In some embodiments:
the compound has formula (IA-A), (IA-AA), or (IA-AB);
ring A is a saturated, monocyclic, 5-membered ring having formula (III-A) (i.e., A₁ in formula (III) is a bond);
L has formula (VIII): —(CH₂)ₘ—X¹—(CH₂)ₙ—X²—(CH₂)ₚ—,
W is represented by formula (X), (XII), (XIII), (XIV), (XIV-A), (XIV-B), or any of SEQ ID 1-27 (e.g., SEQ ID 5-27, e.g., SEQ ID 6); and
m is 2-6 (e.g., 2), X¹ is —N(R′)C(O)— (e.g., —N(H)C(O)—), and n+p=2 (e.g., each of n and p is 1), and X² is —S—; or
m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl; or
m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃); or
m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.

In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI).

In certain of these embodiments, m is 2-6 (e.g., 2), X¹ is —N(R′)C(O)— (e.g., —N(H)C(O)—), and n+p=2 (e.g., each of n and p is 1), and X² is —S—. For example, L can be —CH₂CH₂NHC(O)CH₂SCH₂—.

In certain of these embodiments, any one of SEQ ID 5-27 (e.g., SEQ ID 6) represents W.
[13A] In some embodiments:
the compound has formula (IA-A), (IA-AA), or (IA-AB);
ring A is a saturated, monocyclic, 5-membered ring having formula (III-A) (i.e., A₁ in formula (III) is a bond);
L has formula (VIII): —(CH₂)ₘ—X¹—(CH₂)ₙ—X²—(CH₂)ₚ—, and
W is represented by formula (X), (XII), (XIII), (XIV-AA), (XIV), (XIV-A), (XIV-B), (XV), or any of SEQ ID 1-34 (e.g., SEQ. ID 5-27; e.g., SEQ ID 5-29; (e.g., SEQ ID 6); e.g., SEQ ID 28-29; e.g., SEQ ID 30-34).

In certain embodiments, mis 2-6 (e.g., 2), X¹ is —N(R′)C(O)— (e.g., —N(H)C(O)—), and n+p=2 (e.g., each of n and p is 1), and X² is —S—; or
m is 2-6 (e.g., 2), X¹ is —N(R′)C(O)— (e.g., —N(H)C(O)—), n+p=0, X² is —C(Q¹)(Q²)-, and Q¹ and Q², together with the carbon atom to which each is attached forms C₃₋₆ cycloalkyl that is optionally substituted with from 1-4 independently selected R^{d} and optionally fused to phenyl; or
m is 2-6 (e.g., 2), X¹ is —N(R′)C(O)— (e.g., —N(H)C(O)—), n+p=0, X² is —C(Q¹)(Q²)-, and Q¹ and Q² is an independently selected C₁₋₄ alkyl (e.g., CH₃); or
m is 2-6 (e.g., 2), X¹ is —N(R′)C(O)— (e.g., —N(H)C(O)—), n+p=0, and X² is: (v) C₆₋₁₀ arylene (e.g., phenylene) optionally substituted with from 1-5 independently selected R^{c}; or (vii) C₃₋₁₀ cycloalkylene optionally substituted with from 1-4 independently selected R^{d}.

In certain embodiments, m is 2-6 (e.g., 2), X¹ is —N(R′)C(O)— (e.g., —N(H)C(O)—), n+p=0, and X² is: heterocycloalkylene including from 4-6 (e.g., 4) ring atoms, wherein from 1-3 (e.g., 1) ring atoms are each independently selected from the group consisting of N(R^{a}), O, and S; and which is optionally substituted with from 1-5 independently selected R^{d} (e.g., X² can be

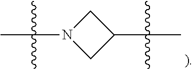 [SMILES: *C1CN(*)C1]

In certain embodiments, one, two, or three of R¹, R², and R⁴ are H. In certain embodiments, R³ is —C(O)OH. In certain embodiments, a is 0, 1, or 2 (e.g., 1). In certain embodiments, R⁵ has formula (XI). In certain embodiments, each of R¹, R², and R⁴ is H, a is 0, 1, or 2 (e.g., 1), R³ is —C(O)OH, and R⁵ has formula (XI). In certain embodiments, a′ is 1; each of R^{3a} and R^{3b} is H.

In certain of these embodiments, m is 2-6 (e.g., 2), X¹ is —N(R′)C(O)— (e.g., —N(H)C(O)—), and n+p=2 (e.g., each of n and p is 1), and X² is —S—. For example, L can be —CH₂CH₂NHC(O)CH₂SCH₂—.

In certain of these embodiments, any one of SEQ ID 5-34 (e.g., SEQ ID 6) represents W.

Embodiments of combinations [1]-[13] can also include any one or more of the features delineated in the Detailed Descriptions and/or claims.

Embodiments of combinations [1A]-[13A] can also include any one or more of the features delineated in the Detailed Descriptions and/or claims.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are contemplated within the scope of this disclosure, though such resonance forms or tautomers are not explicitly represented herein.

### Pharmaceutical Compositions and Administration

### General

In some embodiments, a chemical entity (e.g., a compound or a pharmaceutically acceptable salt and/or hydrate and/or prodrug of the compound) that modulates (e.g., agonizes or partially agonizes or antagonizes) glucagon-like peptide-1 receptor (“GLP-1R”) and/or the gastric inhibitory polypeptide receptor (“GIPR”), is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g, intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In general, the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules, sterility is not required. The USP/NF standard is usually sufficient.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treating and the particular compound being employed. Proper dosage for a particular situation can be determined by one skilled in the medical arts. In some cases, the total daily dosage may be divided and administered in portions throughout the day or by means providing continuous delivery.

In some embodiments, the compounds described herein are administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

This disclosure features methods for treating a subject (e.g., a human) having a disease, disorder, or condition in which modulation of GLP-1R and/or GIPR activities (e.g, repressed or impaired and/or elevated or unwanted GLP-1R and/or GIPR signaling) is beneficial for the treatment of the underlying pathology and/or symptoms and/or progression of the disease, disorder, or condition. In certain embodiments, the methods described herein can include or further include treating one or more conditions associated, co-morbid or sequela with any one or more of the conditions described herein.

In certain embodiments, the chemical entities described herein induce blood glucose reduction (e.g., reduce blood glucose levels), promote insulin synthesis, stimulate insulin secretion, increase the mass of B-cells, modulate gastric acid secretion, modulate gastric emptying, and/or decrease glucagon production. In certain embodiments, the chemical entities described herein stabilize serum glucose and serum insulin levels.

### Indications

### Obesity

In some embodiments, the condition, disease or disorder is obesity and conditions, diseases or disorders that are associated with obesity. Non-limiting examples of obesity and obesity related conditions include symptomatic obesity, simple obesity, childhood obesity, morbid obesity and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, disease or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In certain embodiments, the chemical entities described herein can be used to treat subjects exhibiting symptoms of both obesity and insulin deficiency.

### Diabetes

In some embodiments, the condition, disease or disorder is diabetes. Non-limiting examples of diabetes include type 1 diabetes, type 2 diabetes (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In certain embodiments, the compounds described herein are also useful in improving the therapeutic effectiveness of metformin.

### Disorders of Metabolically Important Tissues

In some embodiments, the condition, disease or disorder is a disorder of a metabolically important tissue.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g, metabolic syndrome (including obesity, diabetes and hypertriglyceridemia) and insulin resistance. It can cause liver disease in adults and children and may ultimately lead to cirrhosis (Skelly et al., J Hepatol 2001; 35:195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl: S186-90). In certain embodiments, the subject is a pediactric subject (e.g., 6-16 years old; or 6-12 years old; or 6-10 years old). In certain embodiments, the subject is a adult subject.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging pregnancy, protection against bone fractures, and malnutritionpolycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction) and geriatric syndrome. In certain embodiments, the chemical entities described herein can be used for treating surgical trauma by improving recovery after surgery and/or by preventing the catabolic reaction caused by surgical trauma.

### Cardiovascular Diseases

In some embodiments, the condition, disease or disorder is a cardiovascular disease. Non-limiting examples of cardiovascular disease include congestive heart failure, atherosclerosis, arteriosclerosis, coronary heart disease, or peripheral artery disease, stroke, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

### Neurological Diseases

In some embodiments, the condition, disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), chronic wasting syndrome). See, e.g., US20060275288A1.

Non-limiting examples of psychiatric disorders include drug dependence/addiction (narcotics and amphetamines and attention deficit/hyperactivity disorder (ADHD). The chemical entities described herein can be useful in improving behavioral response to addictive drugs, decreasing drug dependence, prevention drug abuse relapse, and relieving anxiety caused by the absence of a given addictive substance. See, e.g., US20120021979A1.

In certain embodiments, the chemical entities described herein are useful in improving learning and memory by enhancing neuronal plasticity and facilitation of cellular differentiation, and also in preserving dopamine neurons and motor function in Morbus Parkinson.

### Insulin-Related

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidaemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In certain embodiments, the chemical entities described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease. See, e.g., US20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, cachexia caused by acquired immunodeficiency syndrome).

### Body Weight

In some embodiments, the chemical entities described herein can be used to reduce body weight (e.g., excess body weight), prevent body weight gain, induce weight loss, decrease body fat, or reduce food intake in a subject (e.g., a subject in need thereof). In certain embodiments, the weight increase in a subject may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). Alternatively, the weight increase may be weight increase before reaching obesity, or may be weight increase in an obese subject. The weight increase may also be medication-induced weight gain or weight gain subsequent to cessation of smoking.

In some embodiments, the condition, disease or disorder is an eating disorder, such as hyperphagia, binge eating, bulimia, or compulsive eating.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignantlymphoma, malignantmelanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., diet therapy for diabetes), an exercise therapy, blood sugar monitoring, and diet modifications.

In some embodiments, the compound described herein can be administered in combination with one or more of additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, and therapeutic agents for dysuria.

In some embodiments, the one or more additional therapeutic agents include those useful, e.g., as anti-obesity agents. Non-limiting examples include:
monoamine uptake inhibitors (e.g., tramadol, phentermine, sibutramine, mazindol, fluoxetine, tesofensine);
serotonin 2C receptor agonists (e.g., lorcaserin);
serotonin 6 receptor antagonists;
histamine H3 receptor modulator;
GABA modulator (e.g., topiramate), including GABA receptor agonists (e.g., gabapentin, pregabalin);
neuropeptide Y antagonists (e.g., velneperit);
cannabinoid receptor antagonists (e.g., rimonabant, taranabant);
ghrelin antagonists;
ghrelin receptor antagonists;
ghrelin acylation enzyme inhibitors;
opioid receptor antagonists (e.g., GSK-1521498);
orexin receptor antagonists;
melanocortin 4 receptor agonists;
11β-hydroxysteroid dehydrogenase inhibitors (e.g., AZD-4017, BVT-3498, INCB-13739);
pancreatic lipase inhibitors (e.g., orlistat, cetilistat);
β3 agonists (e.g., N-5984);
diacylglycerol acyltransferase 1 (DGAT1) inhibitors;
acetylCOA carboxylase (ACC) inhibitors;
stearoyl-CoA desaturated enzyme inhibitors;
microsomal triglyceride transfer protein inhibitors (e.g., R-256918);
Na-glucose cotransporter 2 (SGLT-2) inhibitors (e.g., JNJ-28431754, dapagliflozin, AVE2268, TS-033, YM543, TA-7284, ASP1941, remogliflozin);
NFK inhibitors (e.g., HE-3286);
PPAR agonists (e.g., GFT-505, DRF-11605, gemfibrozil and fenofibrate);
phosphotyrosine phosphatase inhibitors (e.g., sodium vanadate, trodusquemin);
GPR119 agonists (e.g., PSN-821, MBX-2982, APD597);
glucokinase activators (e.g., piragliatin, AZD-1656, AZD6370, TTP-355, compounds described in W0006/112549, W0007/028135, W0008/047821, W0008/050821, W0008/136428 and W0008/156757);
leptin, leptin derivatives (e.g., metreleptin), leptin resistance improving drugs;
CNTF (ciliary neurotrophic factor);
BDNF (brain-derived neurotrophic factor);
cholecystokinin agonists, amylin preparations (e.g., pramlintide, AC-2307);
neuropeptide Y agonists (e.g., PYY3-36, derivatives of PYY3-36, obineptide, TM-30339, TM-30335);
oxyntomodulin (OXM) preparations;
appetite suppressants (e.g. ephedrine);
FGF21 preparations (e.g., animal FGF21 preparations extracted from the pancreas of bovine or swine; human FGF21 preparations genetically synthesized using Escherichia coli or yeast; fragments or derivatives of FGF21); and
anorexigenic agents (e.g., P-57).

In some embodiments, the one or more additional therapeutic agents include those useful, e.g., as anti-diabetic agents. Non-limiting examples include:
insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin);
insulin sensitizers (e.g., pioglitazone or a salt thereof);
biguanides (e.g., metformin, buformin or a salt thereof (e.g., hydrochloride, fumarate, succinate));
glucagon analogs (e.g., any of glucagon analogs described, e.g., in WO 2010/011439);
agents which antagonize the actions of or reduce secretion of glucagon;
sulfonylurea agents (e.g., chlorpropamide, tolazamide, gliclazide, glimepiride, tolbutamide, glibenclamide, gliclazide, acetohexamide, glyclopyramide, glybuzole, glyburide);
thiazolidinedione agents (e.g. rosiglitazone or pioglitazone);
α-glucosidase inhibitors (e.g., voglibose, acarbose, miglitol, emiglitate);
insulin secretagogues, such as prandial glucose regulators (sometimes called “short-acting secretagogues”), e.g., meglitinides (e.g. repaglinide and nateglinide);
cholinesterase inhibitors (e.g., donepezil, galantamine, rivastigmine, tacrine);
NMDA receptor antagonists;
dual GLP-1/GIP receptor agonists (e.g., LBT-2000, ZPD1-70);
GLP-1R agonists (e.g., exenatide, liraglutide, albiglutide, dulaglutide, AVE-0010, S4P and Boc5); and
dipeptidyl peptidase IV (DPP-4) inhibitors (e.g., vildagliptin, dutogliptin, gemigliptin, alogliptin, saxagliptin, sitagliptin, linagliptin, berberine, adogliptin, BI1356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, e.g., for treating NAFL and NASH. Non-limiting examples include glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, e.g., for treating diabetic complications. Non-limiting examples include:
aldose reductase inhibitors (e.g., tolrestat, epalrestat, zopolrestat, fidarestat, CT-112, ranirestat, lidorestat);
neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365);
PKC inhibitors (e.g., ruboxistaurin mesylate);
AGE inhibitors (e.g., ALT946, N-phenacylthiazolium bromide (ALT766), EXO-226, pyridorin, pyridoxamine);
serotonin and noradrenalin reuptake inhibitors (e.g., duloxetine);
sodium channel inhibitors (e.g., lacosamide);
active oxygen scavengers (e.g., thioctic acid);
cerebral vasodilators (e.g., tiapuride, mexiletine);
somatostatin receptor agonists (e.g., BIM23 190); and
apoptosis signal regulating kinase-1 (ASK-1) inhibitors.

In some embodiments, the one or more additional therapeutic agents include those useful, e.g., for treating hyperlipidemia. Non-limiting examples include:
HMG-COA reductase inhibitors (e.g., pravastatin, simvastatin, lovastatin, atorvastatin, fluvastatin, rosuvastatin, pitavastatin or a salt thereof (e.g., sodium salt, calcium salt));
squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid);
fibrate compounds (e.g., bezafibrate, clofibrate, simfibrate, clinofibrate);
anion exchange resin (e.g., colestyramine);
nicotinic acid drugs (e.g., nicomol, niceritrol, niaspan);
phytosterols (e.g., soysterol, gamma oryzanol (γ-oryzanol));
cholesterol absorption inhibitors (e.g., zechia);
CETP inhibitors (e.g., dalcetrapib, anacetrapib); and
ω-3 fatty acid preparations (e.g., ω-3-fatty acid ethyl esters 90).

In some embodiments, the one or more additional therapeutic agents include those useful, e.g., as anti-hypertensive agents. Non-limiting examples include:
angiotensin converting enzyme inhibitors (e.g., captopril, enalapril, delapril);
angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil);
calcium antagonists (e.g., manidipine, nifedipine, amlodipine, efonidipine, nicardipine, cilnidipine); and
β-blockers (e.g., metoprolol, atenolol, propranolol, carvedilol, pindolol).

In some embodiments, the one or more additional therapeutic agents include those useful, e.g., as diuretics. Non-limiting examples include:
xanthine derivatives (e.g., theobromine sodium salicylate, theobromine calcium salicylate);
thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide);
antialdosterone preparations (e.g., spironolactone, triamterene);
carbonic anhydrase inhibitors (e.g., acetazolamide); and
chlorobenzenesulfonamide agents (e.g., chlortalidone, mefruside, indapamide).

In some embodiments, the one or more additional therapeutic agents include those useful, e.g., as immunotherapeutic agents. Non-limiting examples include: microbial or bacterial compounds (e.g., muramyl dipeptide derivative, picibanil); polysaccharides having immunoenhancing activity (e.g., lentinan, sizofiran, krestin); cytokines obtained by genetic engineering approaches (e.g., interferon, interleukin (IL) such as IL-1, IL-2, IL-12); and colony-stimulating factors (e.g., granulocyte colony-stimulating factor, erythropoietin).

In some embodiments, the one or more additional therapeutic agents include those useful, e.g., as anti-thrombotic agents. Non-limiting examples include: heparins (e.g., heparin sodium, heparin calcium, enoxaparin sodium, dalteparin sodium); warfarin (e.g., warfarin potassium); anti-thrombin drugs (e.g., aragatroban, dabigatran); FXa inhibitors (e.g., rivaroxaban, apixaban, edoxaban, YM150, compounds described in WO02/06234, WO2004/048363, WO2005/030740, WO2005/058823, and WO2005/113504); thrombolytic agents (e.g., urokinase, tisokinase, alteplase, nateplase, monteplase, pamiteplase); and platelet aggregation inhibitors (e.g., ticlopidine hydrochloride, clopidogrel, prasugrel, E5555, SHC530348, cilostazol, ethyl icosapentate, beraprost sodium, sarpogrelate hydrochloride).

In some embodiments, the one or more additional therapeutic agents include those useful, e.g., for treating osteoporosis. Non-limiting examples include: alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g, distigmine). Suitable examples of anti-inflammatory agents include nonsteroidal anti-inflammatory drugs such as aspirin, acetaminophen, indomethacin.

Other additional therapeutic agents include:
agents that modulate hepatic glucose balance (e.g., fructose 1,6-bisphosphatase inhibitors, glycogen phosphorylase inhibitors, glycogen synthase kinase inhibitors, glucokinase activators);
agents designed to treat the complications of prolonged hyperglycemia, such as aldose reductase inhibitors (e.g. epalrestat and ranirestat);
agents used to treat complications related to micro-angiopathies;
anti-dyslipidemia agents, such as HMG-COA reductase inhibitors (statins, e.g. rosuvastatin);
cholesterol-lowering agents;
bile acid sequestrants (e.g., cholestyramine);
cholesterol absorption inhibitors (e.g. plant sterols such as phytosterols);
cholesteryl ester transfer protein (CETP) inhibitors;
inhibitors of the ileal bile acid transport system (IBAT inhibitors);
bile acid binding resins;
nicotinic acid (niacin) and analogues thereof;
anti-oxidants, such as probucol;
omega-3 fatty acids;
antihypertensive agents, including adrenergic receptor antagonists, such as beta blockers (e.g. atenolol), alpha blockers (e.g. doxazosin), and mixed alpha/beta blockers (e.g. labetalol);
adrenergic receptor agonists, including alpha-2 agonists (e.g. clonidine);
angiotensin converting enzyme (ACE) inhibitors (e.g. lisinopril);
calcium channel blockers, such as dihydropridines (e.g. nifedipine), phenylalkylamines (e.g. verapamil), and benzothiazepines (e.g. diltiazem);
angiotensin II receptor antagonists (e.g. candesartan);
aldosterone receptor antagonists (e.g. eplerenone);
centrally acting adrenergic drugs, such as central alpha agonists (e.g. clonidine);
diuretic agents (e.g. furosemide);
haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban);
antiplatelet agents (e.g., cyclooxygenase inhibitors (e.g. aspirin));
adenosine diphosphate (ADP) receptor inhibitors (e.g. clopidogrel);
phosphodiesterase inhibitors (e.g. cilostazol);
glycoprotein IIB/IIA inhibitors (e.g. tirofiban);
adenosine reuptake inhibitors (e.g. dipyridamole);
noradrenergic agents (e.g. phentermine);
serotonergic agents (e.g. sibutramine);
diacyl glycerolacyltransferase (DGAT) inhibitors;
feeding behavior modifying agents;
pyruvate dehydrogenase kinase (PDK) modulators;
serotonin receptor modulators;
monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine);
compounds described in W0007/013694, WO2007/018314, WO2008/093639 and WO2008/099794;
GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931);
SGLT1 inhibitors;
adiponectin or agonist thereof;
IKK inhibitors (e.g., AS-2868);
somatostatin receptor agonists;
ACC2 inhibitors;
cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M;
metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα);
MC4r agonists;
insulin receptor agonist;
PDE 5 inhibitors;
glycation inhibitors (e.g., ALT-711);
nerve regeneration-promoting drugs (e.g., Y-128, VX853, prosaptide);
antidepressants (e.g., desipramine, amitriptyline, imipramine);
antiepileptic drugs (e.g., lamotrigine, trileptal, keppra, zonegran, pregabalin, harkoseride, carbamazepine);
antiarrhythmic drugs (e.g., mexiletine);
acetylcholine receptor ligands (e.g., ABT-594);
endothelin receptor antagonists (e.g., ABT-627);
narcotic analgesics (e.g., morphine);
α2 receptor agonists (e.g., clonidine);
local analgesics (e.g., capsaicin);
antianxiety drugs (e.g., benzothiazepine);
phosphodiesterase inhibitors (e.g., sildenafil);
dopamine receptor agonists (e.g., apomorphine);
cytotoxic antibodies (e.g., T-cell receptor and IL-2 receptor-specific antibodies);
B cell depleting therapies (e.g., anti-CD20 antibody (e.g., rituxan), i-BLyS antibody);
drugs affecting T cell migration (e.g., anti-integrin alpha 4/beta 1 antibody (e.g., tysabri);
drugs that act on immunophilins (e.g., cyclosporine, tacrolimus, sirolimus, rapamicin);
interferons (e.g., IFN-β);
immunomodulators (e.g., glatiramer);
TNF-binding proteins (e.g., circulating receptors);
immunosupressants (e.g., mycophenolate); and
metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

### Patient Selection

In some embodiments, the methods described herein further include the step of identifying a subject (e.g., a patient) in need of such treatment (e.g., by way of biopsy, endoscopy, or other conventional method known in the art).

### Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, intermediates useful for preparing the compounds described herein can be prepared using the chemistries delineated in any one or more of the following schemes and non-limiting examples.

### A. Abbreviations

AcOH=Acetic acid
BOP=(Benzotriazol-1-yloxy)tris (dimethylamino)phosphonium hexafluorophosphate
CDI=1,1-carbonyldiimidazole
DCC=N,N-dicyclohexylcarbodiimide
DCM=Dichloromethane
DIAD=Diisopropylazodicarboxylate
DIPEA=N,N-diisopropylethylamine
DMAP=4-Dimethylaminopyridine
DMF=N,N-dimethylformamide
DMSO=Dimethylsulfoxide
EDC=1-Ethyl-3-(3-dimethylaminopropyl) carbodiimide hydrochloride
ESI-MS=Electrospray ionization mass spectrometry
Et₂O=Diethyl ether
EtOAc=EtOAc
EtOH=Ethanol
HATU=1-[Bis (dimethylamino)methylene]-1H-1,2,3-triazolo[4,5-b]pyridinium 3-oxide hexafluorophosphate
HOSu=N-hydroxysuccinimide
HPLC=High performance liquid chromatography
LC-MS=Liquid chromatography-mass spectrometry
MeCN=Acetonitrile
MeOH=Methanol
NMR=Nuclear magnetic resonance
Proton sponge=1,8-Bis (dimethylamino) naphthalene
TBAB=Tetrabutylammonium bromide
TBAF=Tetrabutylammonium fluoride
TBAI=Tetrabutylammonium iodide
TBSCl=tert-Butyldimethylsilyl chloride
TEA=Triethylamine
TFA=Trifluoroacetic acid
TFAA=Trifluoroacetic anhydride
THF=Tetrahydrofuran
TIS=Triisopropylsilane
TIPS=Triisopropylsilyl
T3P=Propylphosphonic anhydride
TLC=Thin layer chromatography

### B. Peptide Sequences of Compounds Described in this Invention (SEQ ID NOS 36-58 Disclosed Below, Respectively, in Order of Appearance)

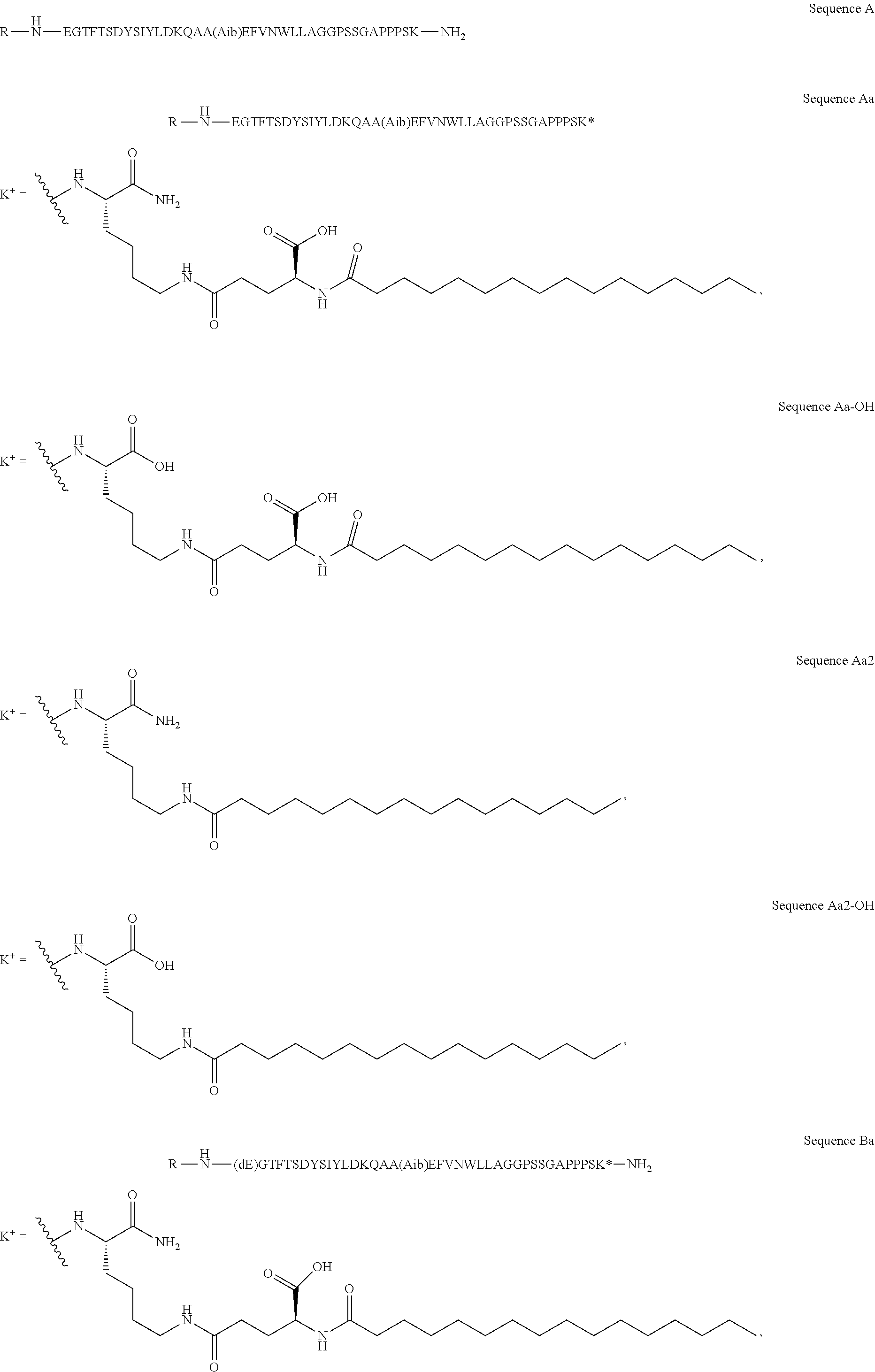

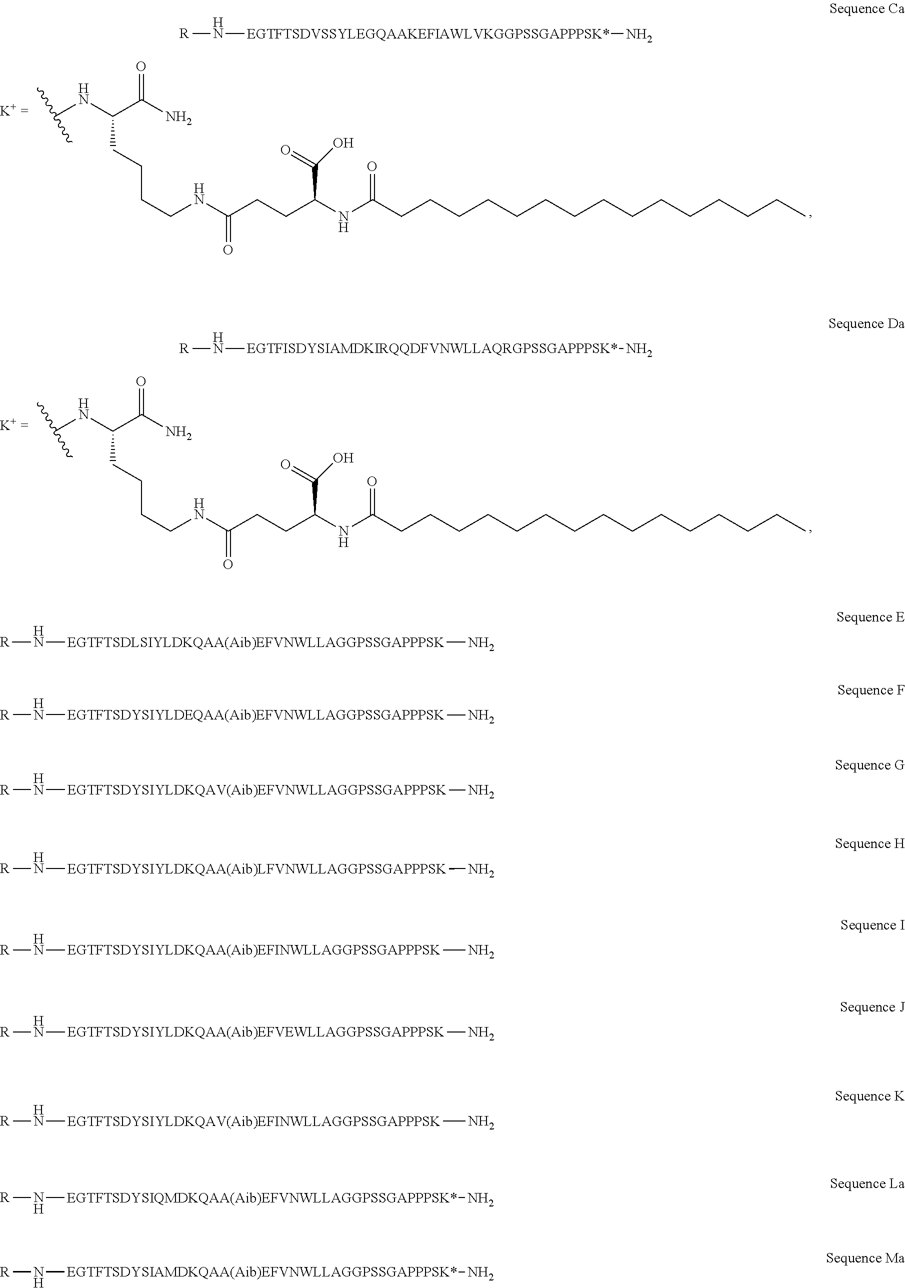

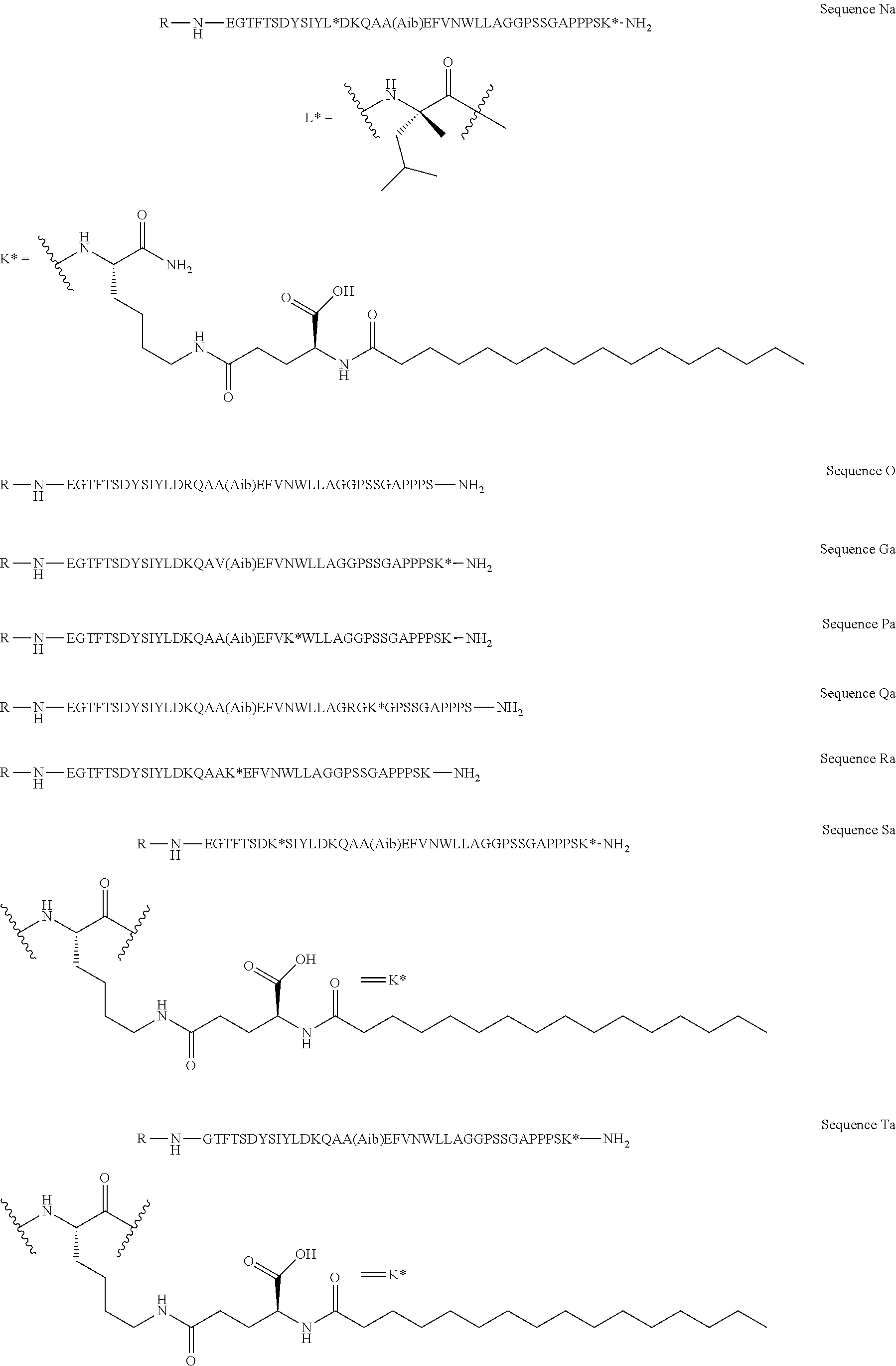

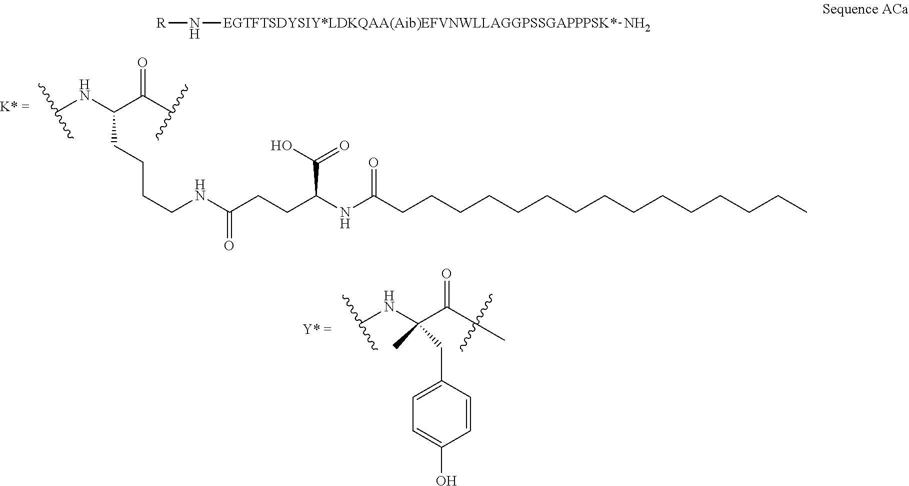

In the sequences depicted above with the exception of Sequence Ta,

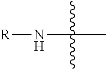

corresponds to the

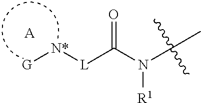

moiety in any of the formulae described herein (wherein R¹ is H).

In sequence Ta above,

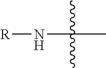

corresponds to the

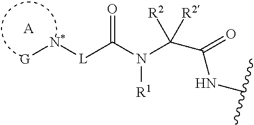

moiety in any of the formulae described herein (wherein R⁴ is H).

### C. Synthesis of Intermediates to Compounds Described in this Invention

### General Procedure 1 (GP1): Preparation of Carboxylic Acid Building Blocks for Peptide N-Terminal Derivatization

To amine or amine salt (1.0 equiv.) in DMF (1-2 mL) was added thiodiglycolic anhydride, 3-methylglutaric anhydride, or 3,3-dimethylglutaric anhydride (1.1-1.2 equiv), followed by DIPEA (2-5 equiv.). After 0.5-16 hours at ambient temperature, the reaction product was isolated by preparative HPLC (Phenomenex Jupiter 10 μM Proteo 90 Å LC column, 250×21.2 mm, with flow rate-15 mL/min, H₂O/MeCN with 0.1% TFA).

### General Procedure 2 (GP2): Preparation of Bis-Pentafluorophenyl) Esters

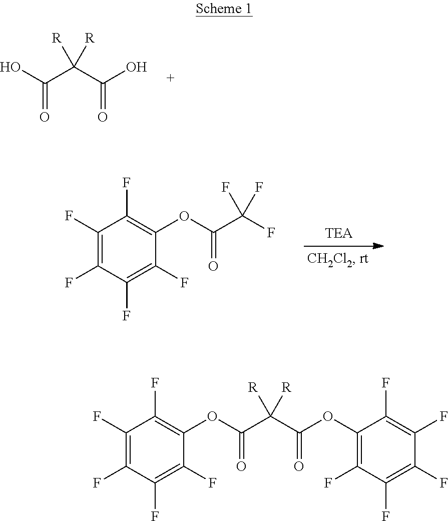

To a slurry of diacid (1.8-3.9 mmol) and triethylamine (2.1-3.0 equiv.) in DCM (5-20 mL) was added pentafluorophenyl trifluoroacetate (2.0-2.1 equiv.). The reaction mixture was stirred at ambient temperature for 5-18 hours, then concentrated and purified by silica gel chromatography to afford the desired diester.

### General Procedure 3 (GP3): Preparation of Pentafluorophenyl Ester Building Blocks From Bis-(Pentafluorophenyl) Esters (as Depicted in Scheme 2)

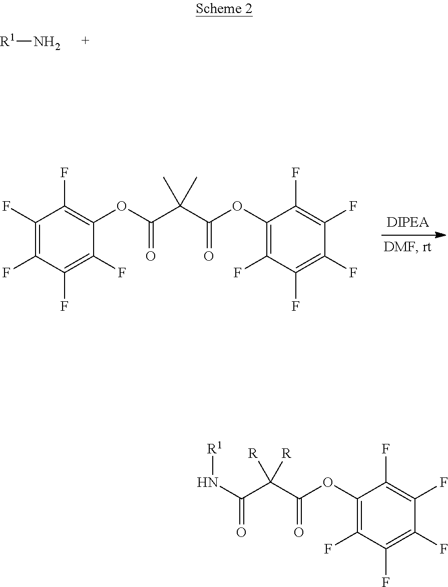

To amine (0.1-0.4 mmol) and bis-(pentafluorophenyl) ester (1.0 equiv.) in DMF (1 mL), added DIPEA (3.0 equiv.). After 3 hours at ambient temperature, the reaction mixture was purified by preparative HPLC (Phenomenex Jupiter 10 μM Proteo 90 Å LC column, 250×21.2 mm, with flow rate—15 mL/min, H₂O/MeCN with 0.1% TFA) to afford the desired substitution product.

### Synthesis of I-3 is Depicted in Scheme 3

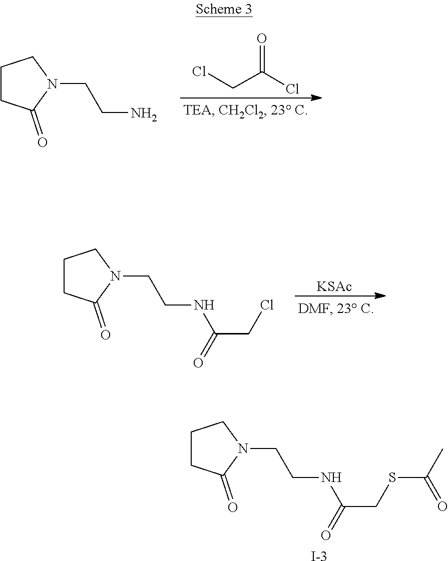

To a solution of 1-(2-aminoethyl)pyrrolidin-2-one (103.1 mg, 0.80 mmol) and triethylamine (0.11 mL, 1.0 equiv.) in DCM (2.3 mL) was added chloroacetyl chloride (63.9 μL, 1.0 equiv.). The reaction mixture was stirred at ambient temperature for 1 hour, then concentrated to a residue which was re-dissolved in DMF (2 mL). Potassium thioacetate (137.7 mg, 1.0 equiv.) was added and the reaction mixture was stirred at ambient temperature for 30 minutes, then concentrated to a residue, which was purified by silica gel chromatography (0-15% MeOH in DCM) to afford I-3. ESI-MS found 245.2, C₁₀H₁₇N₂O₃S (MH⁺) requires 245.1.

### Synthesis of I-5 is Depicted in Scheme 4

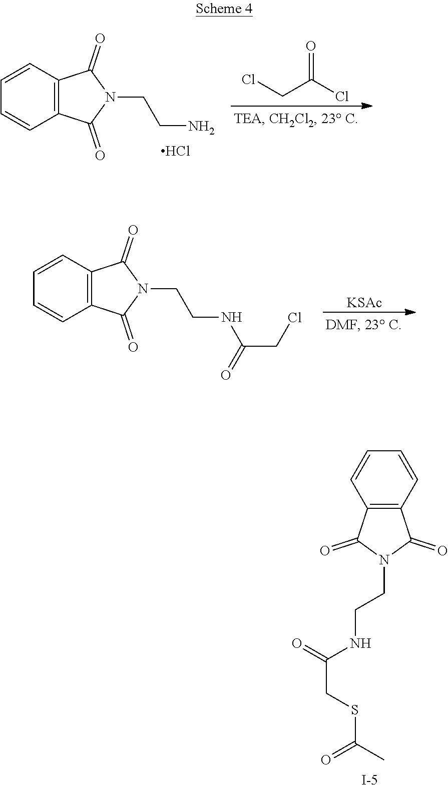

To a solution of 1-(2-aminoethyl)pyrrolidin-2-one (100.5 mg, 0.44 mmol) and triethylamine (61.8 μL, 1.0 equiv.) in DCM (1.1 mL) was added chloroacetyl chloride (35.2 μL, 1.0 equiv.). The reaction mixture was stirred at ambient temperature for 1 hour, then concentrated to a residue which was re-dissolved in DMF (2 mL). Potassium thioacetate (75 mg, 1.0 equiv.) was added and the reaction mixture was stirred at ambient temperature for 30 minutes, then concentrated to a residue, which was purified by silica gel chromatography (0-15% MeOH in DCM) to afford I-5 (69.3 mg, 51%, 2 steps). ESI-MS found 307.2, C₁₄H₁₅N₂O₄S (MH⁺) requires 307.1.

### I-6

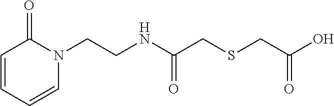 [SMILES: O=C(O)CSCC(=O)NCCn1ccccc1=O]

Prepared according to GP1. Yield: 57.8 mg (64%). ESI-MS found 271.2, C₁₁H₁₅N₂O₄S (MH⁺) requires 271.1.

### I-7

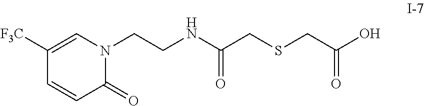 [SMILES: O=C(O)CSCC(=O)NCCn1cc(C(F)(F)F)ccc1=O]

Prepared according to GP1. Yield: 28.6 mg (69%). ESI-MS found 339.2, C₁₂H₁₄F₃N₂O₄S (MH⁺) requires 339.1.

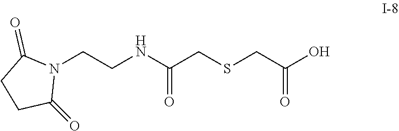 [SMILES: O=C(O)CSCC(=O)NCCN1C(=O)CCC1=O]

Prepared according to GP1. Yield: 70.1 mg (100%). ESI-MS found 275.2, C₁₀H₁₅N₂O₅S (MH⁺) requires 275.1.

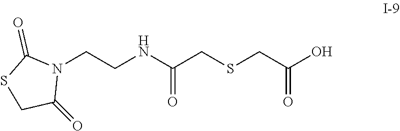 [SMILES: O=C(O)CSCC(=O)NCCN1C(=O)CSC1=O]

Prepared according to GP1. Yield: 188.4 mg (73%). ESI-MS found 292.2, C₉H₁₃N₂O₅S₂ (MH⁺) requires 292.1.

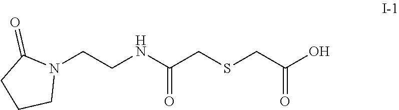 [SMILES: O=C(O)CSCC(=O)NCCN1CCCC1=O]

Prepared according to GP1. Yield: 92.7 mg (68%). ESI-MS found 259.3, C₁₀H₁₅N₂O₄S (M−H⁺) requires 259.1.

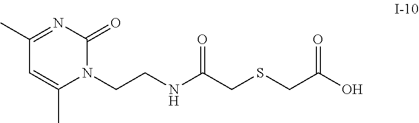 [SMILES: Cc1cc(C)n(CCNC(=O)CSCC(=O)O)c(=O)n1]

Prepared according to GP1. Yield: 73 mg. ESI-MS found 300.2, C₁₂H₁₈N₃O₄S (MH⁺) requires 300.1.

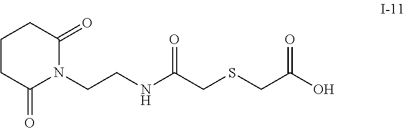 [SMILES: O=C(O)CSCC(=O)NCCN1C(=O)CCCC1=O]

Prepared according to GP1. Yield: 60.1 mg (87%). ESI-MS found 289.2, C₁₁H₁₇N₂O₅S (MH⁺) requires 289.1.

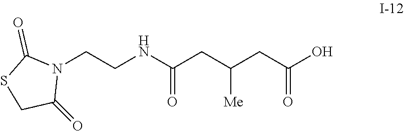 [SMILES: CC(CC(=O)O)CC(=O)NCCN1C(=O)CSC1=O]

Prepared according to GP1. Yield: 68.5 mg (98%). ESI-MS found 289.2, C₁₁H₁₇N₂O₅S (MH⁺) requires 289.1.

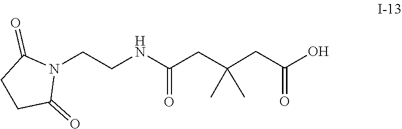 [SMILES: CC(C)(CC(=O)O)CC(=O)NCCN1C(=O)CCC1=O]

Prepared according to GP1. Yield: 73.1 mg (95%). ESI-MS found 303.2, C₁₂H₁₉N₂O₅S (MH⁺) requires 303.1.

### Synthesis of I-15 is Depicted in Scheme 5

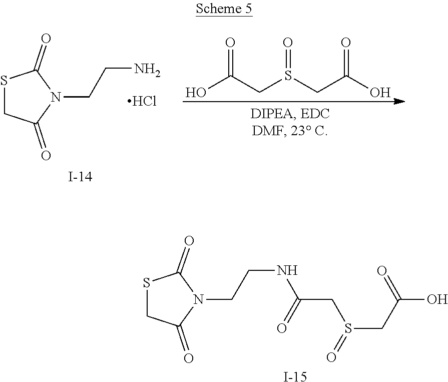

To a solution of amine salt I-14 (65 mg, 0.33 mmol) and 2,2′-sulfinyldiacetic acid (114 mg, 2.1 equiv.) in DMF (1 mL) was added DIPEA (0.12 mL, 2.1 equiv.) and EDC (67 mg, 1.06 equiv.). The reaction mixture was stirred at ambient temperature for 16 hours. Additional DIPEA (0.06 mL, 1.0 equiv.) was then added. After an additional 24 hours, purification by preparative HPLC (H₂O/MeCN with 0.1% TFA) afforded 106.9 mg (quantitative yield) of I-15 as a colorless oil. ESI-MS found 306.9, C₉H₁₁N₂O₆S₂ (M−H⁺) requires 307.0.

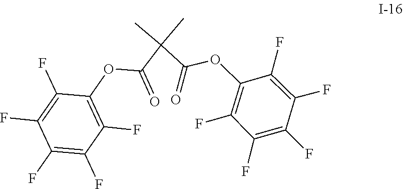 [SMILES: CC(C)(C(=O)Oc1c(F)c(F)c(F)c(F)c1F)C(=O)Oc1c(F)c(F)c(F)c(F)c1F]

Diester I-16: Prepared according to GP2. Yield: 1.16 g (65%).

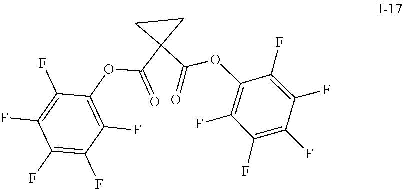 [SMILES: O=C(Oc1c(F)c(F)c(F)c(F)c1F)C1(C(=O)Oc2c(F)c(F)c(F)c(F)c2F)CC1]

Diester I-17: Prepared according to GP2. Yield: 599 mg (29%)

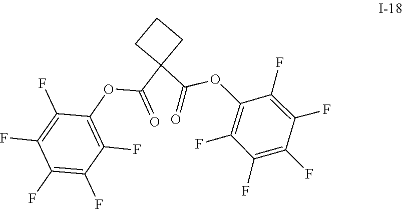 [SMILES: O=C(Oc1c(F)c(F)c(F)c(F)c1F)C1(C(=O)Oc2c(F)c(F)c(F)c(F)c2F)CCC1]

Diester I-18: Prepared according to GP2. Yield: 778 mg (46%)

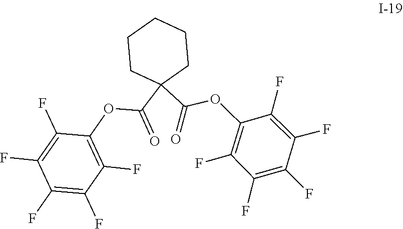 [SMILES: O=C(Oc1c(F)c(F)c(F)c(F)c1F)C1(C(=O)Oc2c(F)c(F)c(F)c(F)c2F)CCCCC1]

Diester I-19: Prepared according to GP2. Yield: 569.8 mg (64%).

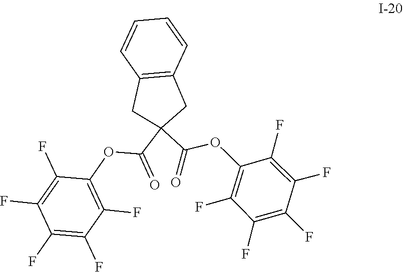 [SMILES: O=C(Oc1c(F)c(F)c(F)c(F)c1F)C1(C(=O)Oc2c(F)c(F)c(F)c(F)c2F)Cc2ccccc2C1]

Diester I-20: Prepared according to GP2. Yield: 399.3 mg (48%).

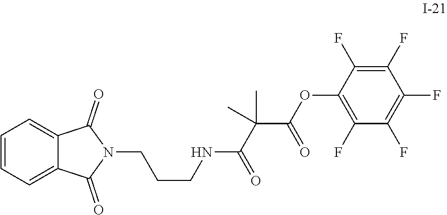 [SMILES: CC(C)(C(=O)NCCCN1C(=O)c2ccccc2C1=O)C(=O)Oc1c(F)c(F)c(F)c(F)c1F]

Prepared according to GP3 from diester I-16. Yield: 43.2 mg (50%). ESI-MS found 485.1, C₂₂H₁₈F₅N₂O₅ (MH⁺) requires 485.1.

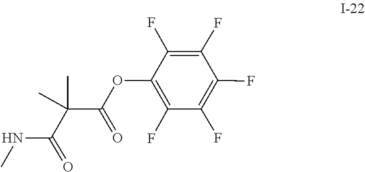 [SMILES: CNC(=O)C(C)(C)C(=O)Oc1c(F)c(F)c(F)c(F)c1F]

Prepared according to GP3 from diester I-16. Yield: 25.8 mg (33%). ESI-MS found 312.1, C₁₂H₁₁F₅NO₃ (MH⁺) requires 312.1.

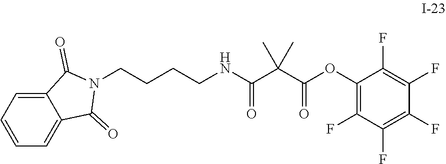 [SMILES: CC(C)(C(=O)NCCCCN1C(=O)c2ccccc2C1=O)C(=O)Oc1c(F)c(F)c(F)c(F)c1F]

Prepared according to GP3 from diester I-16. Yield: 53.4 mg (55%). ESI-MS found 499.1, C₂₃H₂₀F₅N₂O₅ (MH⁺) requires 499.1.

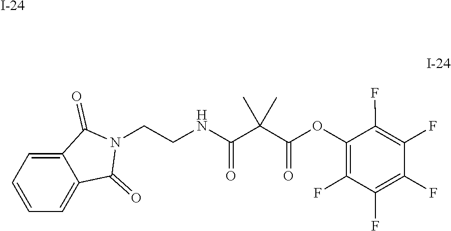

Prepared according to GP3 from diester I-16. Yield: 56.7 mg (58%). ESI-MS found 471.1, C₂₁H₁₆F₅N₂O₅ (MH⁺) requires 471.1.

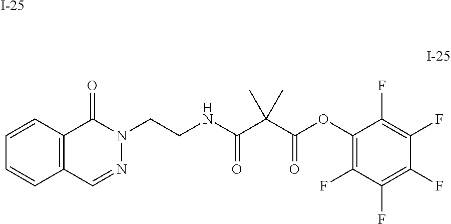

Prepared according to GP3 from diester I-16. Yield: 28.7 mg (33%). ESI-MS found 470.3, C₂₁H₁₇F₅N₃O₄ (MH⁺) requires 470.1.

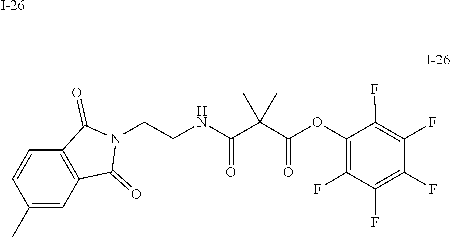

Prepared according to GP3 from diester I-16. Yield: 57.4 mg (86%). ESI-MS found 485.3, C₂₂H₁₈F₅N₂O₅ (MH⁺) requires 485.1.

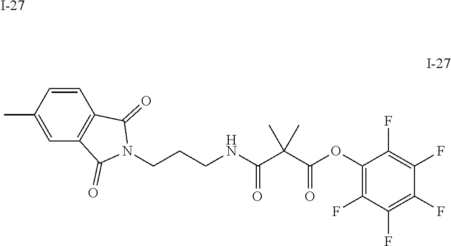

Prepared according to GP3 from diester I-16. Yield: 18.1 mg (27%).

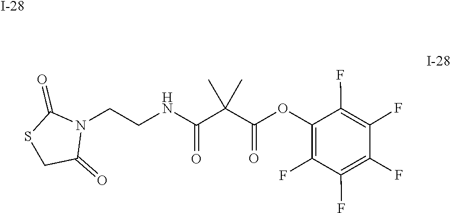

Prepared according to GP3 from diester I-16. Yield: 48 mg (33%). ESI-MS found 441.1, C₁₆H₁₄F₅N₂O₅S (MH⁺) requires 441.1.

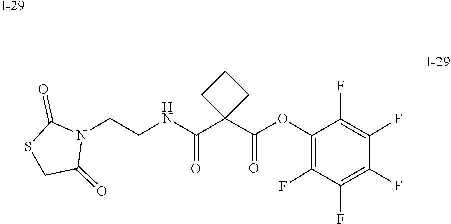

Prepared according to GP3 from diester I-18. Yield: 66.1 mg (59%). ESI-MS found 453.1, C₁₇H₁₄F₅N₂O₅S (MH⁺) requires 453.1.

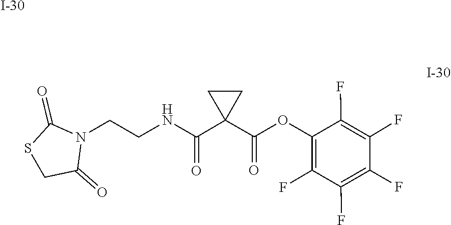

Prepared according to GP3 from diester I-17. Yield: 58.8 mg (48%). ESI-MS found 439.1, C₁₆H₁₂F₅N₂O₅S (MH⁺) requires 439.1.

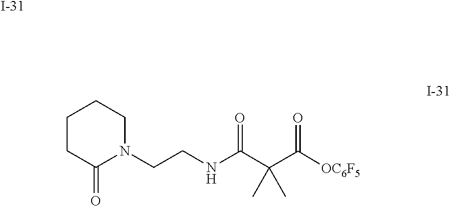

Prepared according to GP3 from diester I-16. Yield: 47.9 mg (61%). ESI-MS found 423.1, C₁₈H₂₀F₅N₂O₄ (MH⁺) requires 423.1.

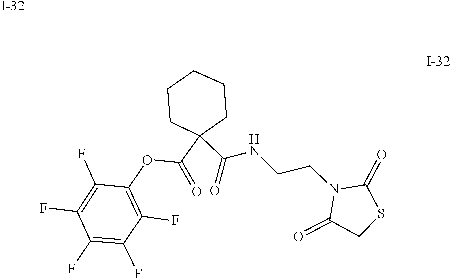

Prepared according to GP3 from diester I-19. Yield: 40 mg (30% yield). ESI-MS found 481.2, C₁₉H₁₈F₅N₂O₅S (MH⁺) requires 481.1.

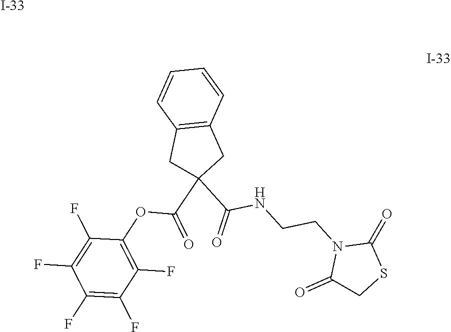

I-33: Prepared according to GP3 from diester I-20. Yield: 8.6 mg (9% yield).

### Synthesis of I-34 is Depicted in Scheme 6

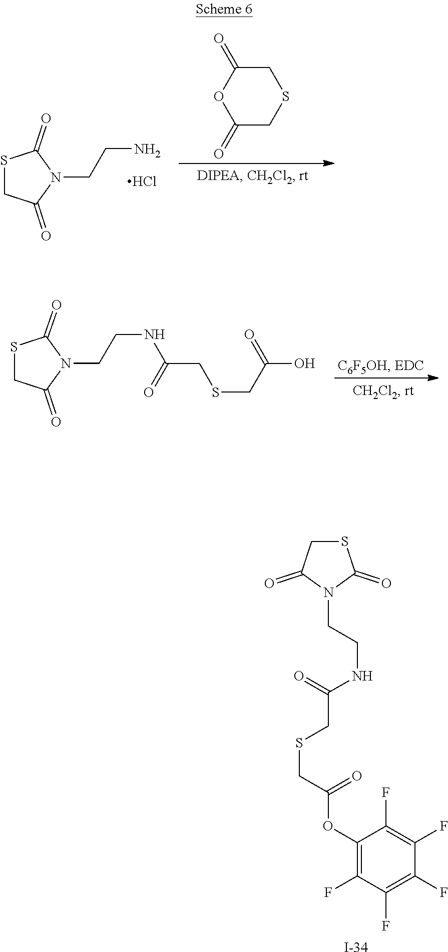

To a slurry of 3-(2-aminoethyl) thiazolidine-2,4-dione hydrochloride (100 mg, 0.51 mmol) and thiodiglycolic anhydride (69 mg, 1.0 equiv.) in DCM (1 mL) was added DIPEA (0.18 mL, 2.0 equiv.). The reaction mixture was stirred at ambient temperature for 2 hours. Pentafluorophenol (98 mg, 1.0 equiv.) was then added, followed by EDC (100 mg, 1.0 equiv.). The reaction mixture was stirred for 16 hours, then concentrated and purified by silica gel chromatography (0-60% EtOAc in Hexanes) to afford 69.1 mg (30% yield) of I-34 as a white solid. ESI-MS found 459.1. C₁₅H₁₂F₅N₂O₅S₂ (MH⁺) requires 459.0.

### Synthesis of I-35 is Depicted in Scheme 7

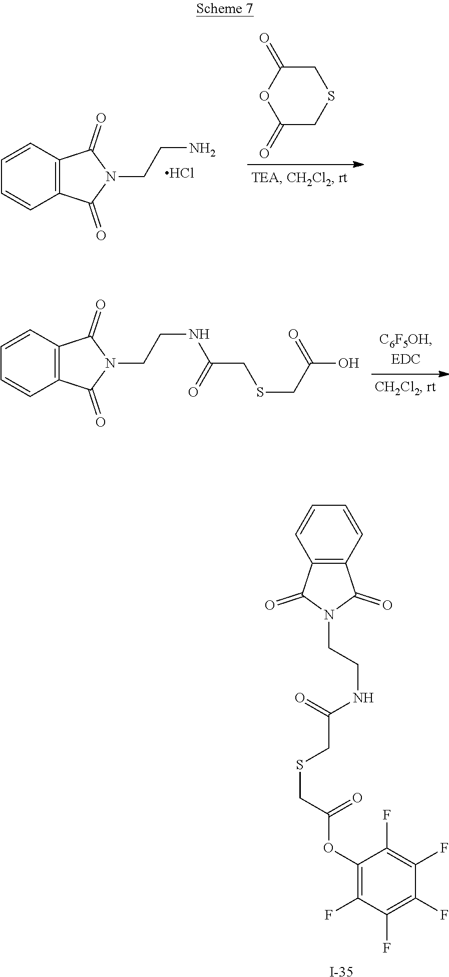

To a slurry of 2-(2-aminoethyl)isoindoline-1,3-dione hydrochloride (200 mg, 0.82 mmol) and thiodiglycolic anhydride (122 mg, 1.05 equiv.) in DCM (5 mL) and DMF (2 mL) was added TEA (0.27 mL, 2.2 equiv.). The reaction mixture was stirred at ambient temperature for 16 hours. Additional thiodiglycolic anhydride (23 mg, 0.2 equiv.) was added for completion of the reaction. The reaction mixture was concentrated and diluted with DCM (30 mL). The organic phase was washed with 1M HCl (30 mL) and the aqueous phase was back-extracted with EtOAc (30 mL). The combined organic extracts were dried (MgSO₄) and concentrated to a residue, which was purified by silica gel chromatography (10:1 DCM/MeOH with 1% AcOH) to afford 400 mg of intermediate acid, This acid was re-dissolved in DCM (5 mL). Pentafluorophenol (215 mg, 1.3 equiv.) was then added, followed by EDC (224 mg, 1.3 equiv.). The reaction mixture was stirred for 1 hour, and additional pentafluorophenol (108 mg, 0.65 equiv.) and EDC (112 mg, 0.65 equiv.) were added. After an additional 30 minutes, the reaction mixture was concentrated and purified by silica gel chromatography (0-100% EtOAc in Hexanes) to afford 250 mg (58% yield, 2 steps) of I-35 as a white solid. ESI-MS found 489.1. C₂₀H₁₄F₅N₂O₅S (MH⁺) requires 489.1.

### Synthesis of I-36 is Depicted in Scheme 8

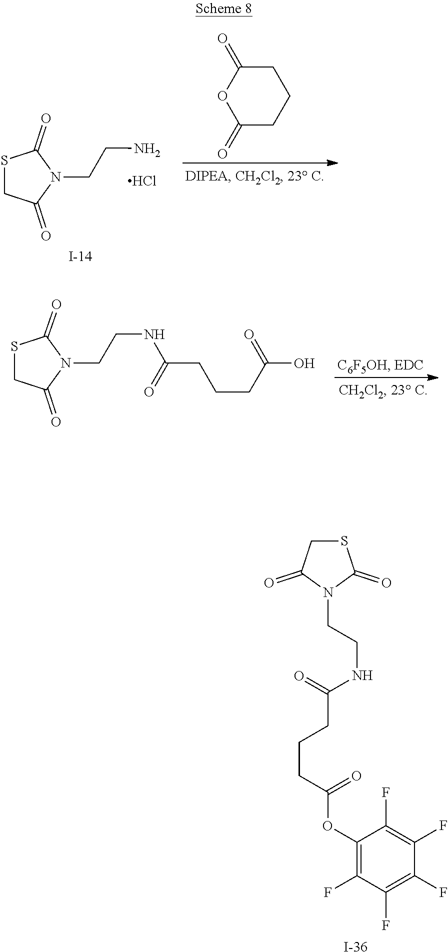

Amine salt I-14 (81 mg, 0.41 mmol) and glutaric anhydride (49 mg, 1.04 equiv.) were suspended in DCM (4 mL), and DIPEA (0.14 mL, 2.0 equiv.) was added. The reaction mixture was stirred at ambient temperature for 15 hours, whereupon pentafluorophenol (80 mg, 1.06 equiv.) and EDC (81 mg, 1.02 equiv.) were added to the homogeneous solution. After an additional 4 hours, the reaction mixture was diluted with DCM (20 mL) and washed with 1M HCl (5 mL) and brine (8 mL). The organic extracts were dried (Na₂SO₄) and concentrated to a residue, which was purified by silica gel chromatography (Hexanes/EtOAc) to afford 105 mg (58% yield) of I-36 as a white solid. ESI-MS found 441.0. C₁₆H₁₄F₅N₂O₅S (MH⁺) requires 441.1.

### Synthesis of I-37 is Depicted in Scheme 9

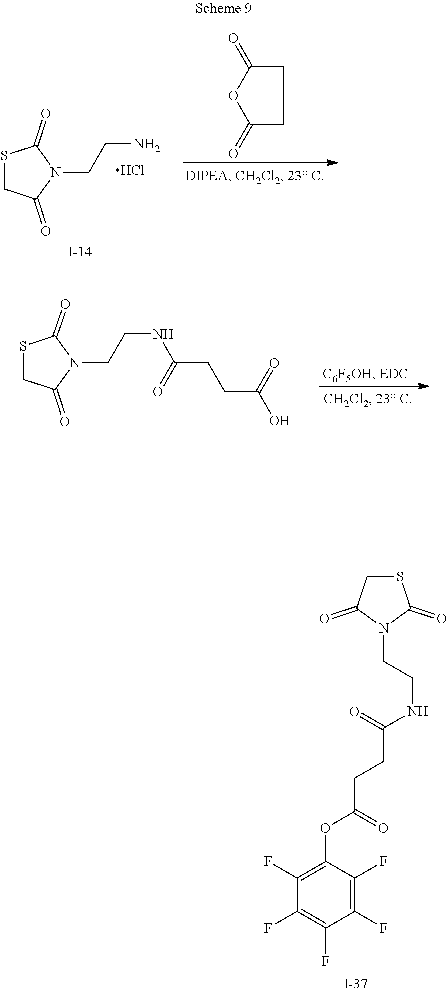

Amine salt I-14 (69 mg, 0.35 mmol) and succinic anhydride (35 mg, 1.0 equiv.) were suspended in DCM (5 mL), and DIPEA (0.12 mL, 2.0 equiv.) was added. The reaction mixture was stirred at ambient temperature for 16 hours, whereupon pentafluorophenol (80 mg, 1.24 equiv.) and EDC (74 mg, 1.1 equiv.) were added to the homogeneous solution. After an additional 4 hours, the reaction mixture was diluted with DCM (20 mL) and washed with 1M HCl (5 mL) and brine (8 mL). The organic extracts were dried (Na₂SO₄) and concentrated to a residue, which was purified by silica gel chromatography (Hexanes/EtOAc) to afford 64 mg (43% yield) of I-37 as a white solid. ESI-MS found 427.0. C₁₅H₁₂F₅N₂O₅S (MH⁺) requires 427.1.

### Synthesis of I-38 is Depicted in Scheme 10

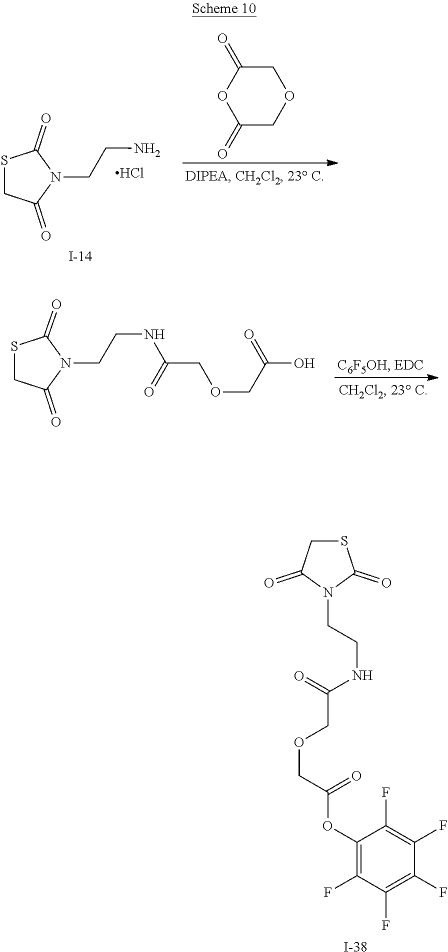

Amine salt I-14 (73 mg, 0.37 mmol) and diglycolic anhydride (43 mg, 1.0 equiv.) were suspended in DCM (5 mL), and DIPEA (0.14 mL, 2.2 equiv.) was added. The reaction mixture was stirred at ambient temperature for 17 hours, whereupon pentafluorophenol (86 mg, 1.26 equiv.) and EDC (78 mg, 1.1 equiv.) were added to the homogeneous solution. After an additional 6 hours, the reaction mixture was diluted with DCM (20 mL) and washed with 1M HCl (5 mL) and brine (8 mL). The organic extracts were dried (Na₂SO₄) and concentrated to a residue, which was purified by silica gel chromatography (Hexanes/EtOAc) to afford 22 mg (14% yield) of I-38 as a white solid. ESI-MS found 443.0. C₁₅H₁₂F₅N₂O₆S (MH⁺) requires 443.0.

### Synthesis of I-39 is Depicted in Scheme 11

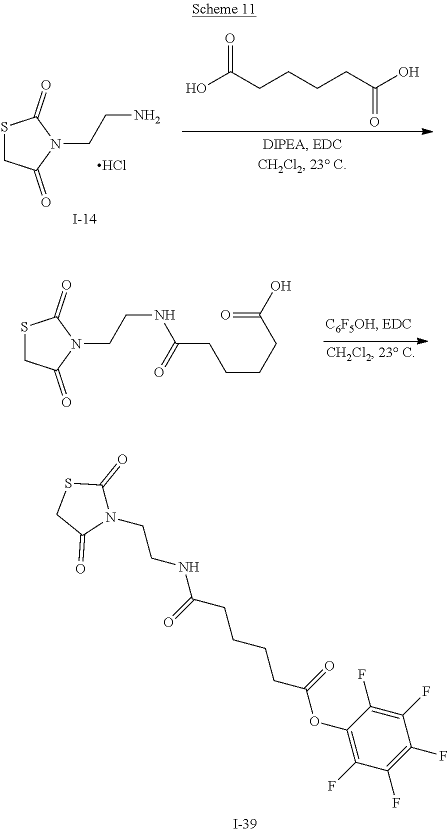

To a suspension of amine salt I-14 (99 mg, 0.50 mmol), adipic acid (73.7 mg, 1.0 equiv.) and EDC (98 mg, 1.02 equiv.) in DCM (5 mL) was added DIPEA (0.27 mL, 3.1 equiv.). The reaction mixture was stirred at ambient temperature for 60 hours at which point pentafluorophenol (98 mg, 1.06 equiv.) and additional EDC (102 mg, 1.05 equiv.) were added to the homogeneous solution. After an additional 4.5 hours, the reaction mixture was diluted with DCM (20 mL) and washed with 1M HCl (5 mL) and brine (8 mL). The organic extracts were dried (Na₂SO₄) and concentrated to a residue, which was purified by silica gel chromatography (Hexanes/EtOAc) to afford 90 mg (39% yield) of I-39 as a white solid. ESI-MS found 455.0. C₁₇H₁₆F₅N₂O₅S (MH⁺) requires 455.1.

### Synthesis of I-40 is Depicted in Scheme 12

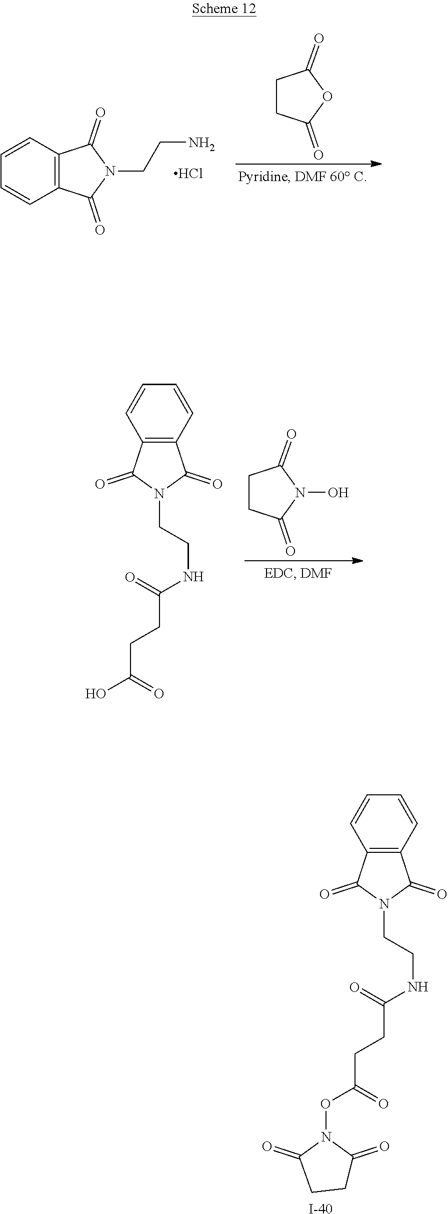

To a slurry of 2-(2-aminoethyl)isoindoline-1,3-dione hydrochloride (74.3 mg, 0.33 mmol) and succinic anhydride (39.4 mg, 1.2 equiv.) in DMF (1.5 mL) was added pyridine (79 μL, 3.0 equiv.). The reaction mixture was heated to 60° C. for 1 hour at which point acylation was complete. The reaction mixture was cooled to ambient temperature, and HOSu (75.5 mg, 2.0 equiv.) was added, followed by EDC (125.8 mg, 2.0 equiv.) The reaction mixture was stirred at ambient temperature for 4 hours. The reaction mixture was diluted saturated aqueous NH₄Cl (8 mL) and H₂O (5 mL) and extracted with EtOAc (3×5 mL). The organic extracts were combined, dried (MgSO₄), and concentrated to afford a white solid. Silica gel chromatography (EtOAc) afforded 22.9 mg (18% yield) of ester I-40.

### Synthesis of I-41 is Depicted in Scheme 13

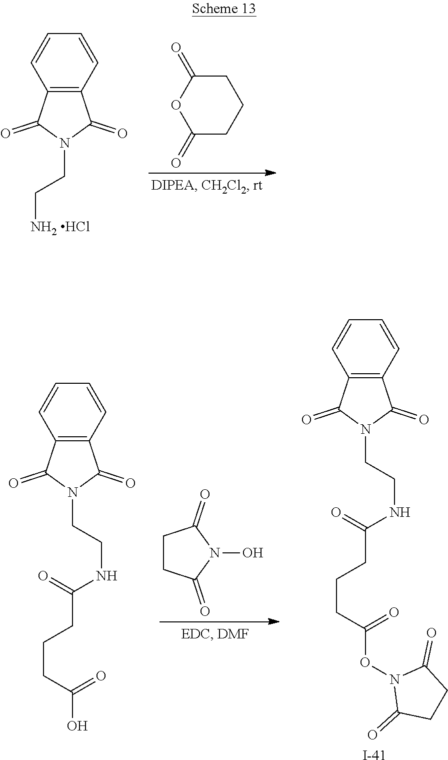

To a solution of 2-(2-aminoethyl)isoindoline-1,3-dione hydrochloride (102.2 mg 0.45 mmol) and glutaric anhydride (61.7 mg, 1.2 equiv.) in DMF (2 mL) was added DIPEA (196 μL, 2.5 equiv.). The reaction mixture was stirred for 22.5 hours at ambient temperature and HOSu (103.8 mg, 2.0 equiv.) was added, followed by EDC (172.8 mg, 2.0 equiv.). The reaction mixture was maintained at ambient temperature for an additional 16 hours, then diluted with H₂O (10 mL) and extracted with EtOAc (3×5 mL). The combined organic extracts were dried (MgSO₄) and concentrated to afford a residue, which was purified by silica gel chromatography (EtOAc) to afford 85.8 mg (47% yield, 2 steps) of I-41 as a white solid. ESI-MS found 402.3. C₁₉H₂₀N₃O₇ (MH⁺) requires 402.1.

### Synthesis of I-42 is Depicted in Scheme 14

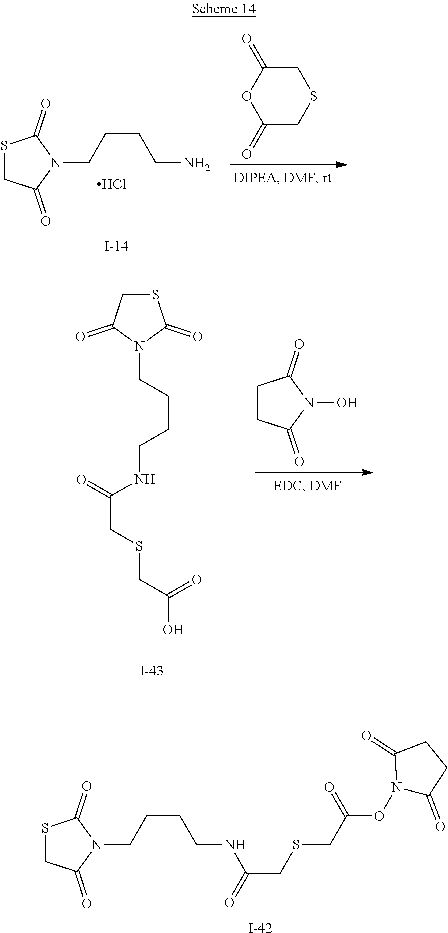

To amine salt I-14 (63.4 mg, 0.28 mmol) in DMF (1 mL) was added thiodiglycolic anhydride (44.7 mg, 1.2 equiv), followed by DIPEA (123 μL, 2.5 equiv.). After stirring at ambient temperature for 3 hours, purification by preparative HPLC (H₂O/MeCN with 0.1% TFA) afforded 70.5 mg (78% yield) of carboxylic acid I-43 as a white solid. Carboxylic acid I-43 was dissolved in DMF (2 mL), and HOSu (38 mg, 1.5 equiv.) was added, followed by EDC (63.3 mg, 1.5 equiv.) The reaction mixture was stirred at ambient temperature for 18 hours, then subjected to preparative HPLC (H₂O/MeCN with 0.1% TFA) to afford 89.8 mg (98% yield) of ester I-42 as a white solid. ESI-MS found 418.2. C₁₅H₂₀N₃O₇S₂ (MH⁺) requires 418.1.

### Synthesis of I-44 is Depicted in Scheme 15

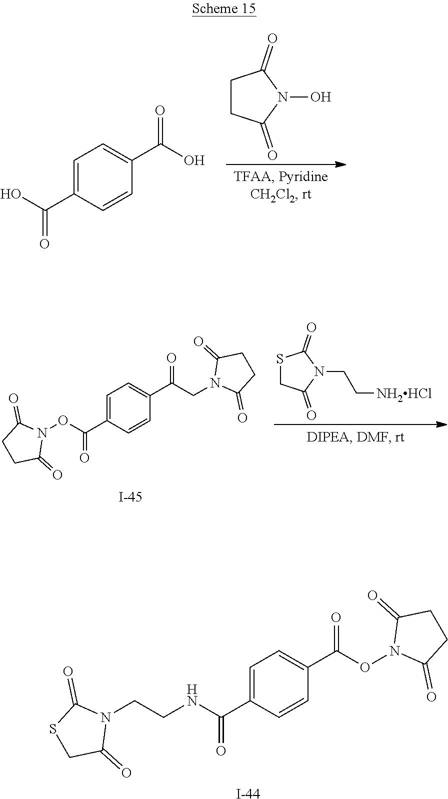

To a slurry of terephthalic acid (216.7 mg, 1.304 mmol) and HOSu (547.9 mg 2.0 equiv.) in DCM (10 mL) was added pyridine (420 mL, 4.0 equiv.). The reaction mixture was cooled to 0-5° C. and trifluoroacetic anhydride (368 mL, 2.0 equiv.) was added. The reaction mixture was allowed to warm to ambient temperature, stirred for 16 hours, and then concentrated. EtOH (20 mL) was added to the residue and the slurry was filtered and dried to afford the crude bis-N-hydroxysuccinimide ester I-45 (403 mg, 86% yield), which was taken forward without further purification.

To a solution of 3-(2-aminoethyl) thiazolidine-2,4-dione hydrochloride (34.3 mg 0.174 mmol) in DMF (1 mL) was added diester I-45 (125.7 mg, 2.0 equiv.) and DIPEA (91 mL, 3.0 equiv.). The reaction mixture was maintained at ambient temperature for 17 hours and then purified by HPLC to afford 54 mg (76% yield) of I-44.

### Synthesis of I-48 is Depicted in Scheme 16

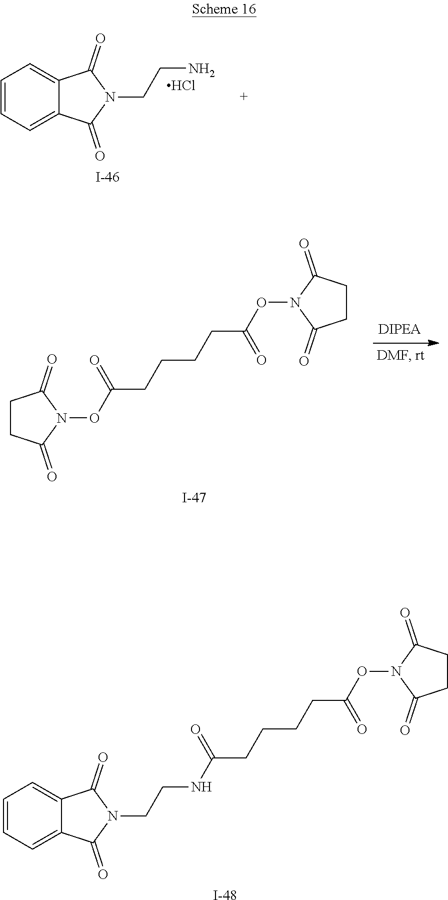

I-48: Adipic acid bis-N-hydroxysuccinimide ester I-47 was prepared as described for diester I-45. Coupling with amine hydrochloride I-46 as described in the preparation of I-44 afforded 69.8 mg (69% yield) of I-48 after purification by preparative HPLC (H₂O/MeCN with 0.1% TFA).

### Synthesis of I-51 is Depicted in Scheme 17

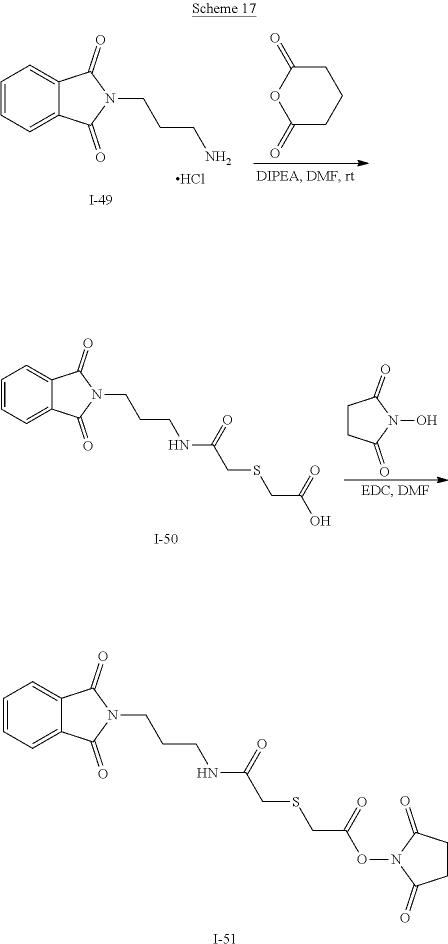

Using a procedure similar to that described for the preparation of I-42, amine hydrochloride I-49 (85 mg, 0.353 mmol) afforded 62.5 mg (38% yield, 2 steps) of I-51.

### Synthesis of I-53 is Depicted in Scheme 18

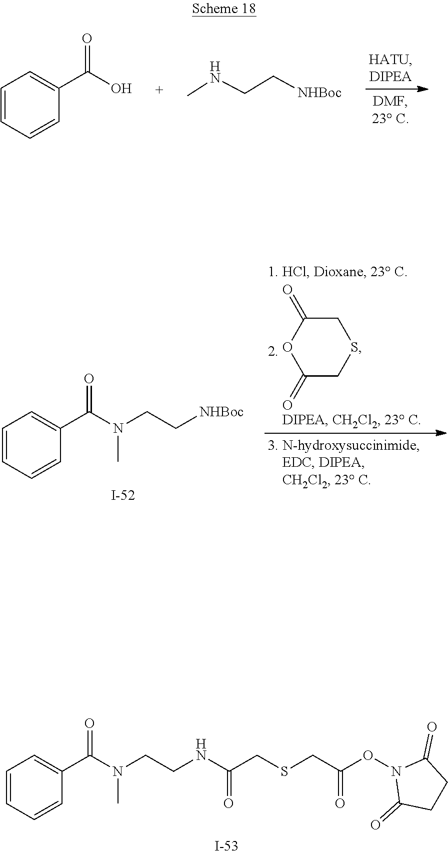

To a solution of benzoic acid (66.2 mg, 0.54 mmol) and tert-butyl (2-(methylamino)ethyl)carbamate (97.5 mg, 1.03 equiv.) in DMF (3 mL) was added HATU (216.8 mg, 1.05 equiv.) and DIPEA (0.1 mL, 1.06 equiv.). The reaction mixture was stirred at ambient temperature for 16 hours, then diluted with EtOAc (20 mL) and washed with water (10 mL), 1M HCl (10 mL), 1M NaOH (10 mL) and brine (10 mL). The combined organic extracts were dried (Na₂SO₄) and concentrated to afford intermediate amide I-52 (130.8 mg), which was then dissolved in 4M HCl in dioxane (3 mL). After 1 hour, the reaction mixture was concentrated to afford the deprotected amine as a solid. This amine was suspended in DCM (3 mL) along with thiodiglycolic anhydride (60.4 mg, 0.97 equiv.), and DIPEA (0.17 mL, 2.1 equiv.) was added. The reaction mixture was stirred at ambient temperature for 2.5 hours, at which point HOSu (55.1 mg, 1.02 equiv.) and EDC (90 mg 1.0 equiv.) were added. After 16 hours, the reaction mixture was diluted with DCM (20 mL) and washed with 1M HCl (2×10 mL) and brine (8 mL). The organic extracts were dried (Na₂SO₄) and concentrated to a residue, which was purified by silica gel chromatography (EtOAc) to afford 34.5 mg (18% yield) of I-53. ESI-MS found 408.2. C₁₈H₂₂N₃O₆S (MH⁺) requires 408.1.

### Synthesis of I-55 is Depicted in Scheme 19

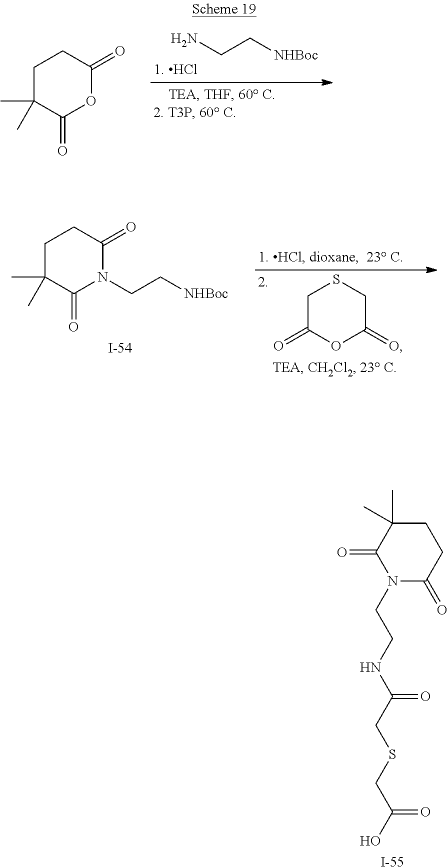

To a solution of 3,3-dimethyldihydro-2H-pyran-2,6(3H)-dione (98.7 mg, 0.69 mmol) and tert-butyl (2-aminoethyl)carbamate hydrochloride (140 mg, 1.02 equiv.) in THF (6 mL) was added TEA (0.2 mL, 2.1 equiv.). The reaction mixture was heated to 60° C. for 48 hours. T3P (50% solution in EtOAc, 0.27 g, 0.6 equiv.) was added, followed by additional THF (3 mL). The reaction mixture was heated to 60° C. for 8 hours, at which time additional T3P (0.47 g, 1.1 equiv.) was added and the reaction mixture was heated to 60° C. for an additional 16 hours. The reaction mixture was diluted with EtOAc (20 mL) and washed with H₂O (2×10 mL), saturated aqueous NaHCO₃(10 mL), 1M HCl (10 mL), and brine (10 mL). The organic extracts were dried (Na₂SO₄) and concentrated to afford a residue, which was purified by silica gel chromatography to afford the glutarimide I-54 (59 mg, 30% yield).

Glutarimide I-54 (59 mg, 0.21 mmol) was dissolved in 4M HCl in dioxane (3 mL). After 2 hours, the reaction mixture was concentrated to afford a white solid. This solid and thiodiglycolic anhydride (28.3 mg, 1.03 equiv.) were dissolved in DCM (3 mL), and TEA (90 μL, 3.1 equiv.) was added. After 25 minutes, the reaction mixture was concentrated and purified by preparative HPLC (H₂O/MeCN with 0.1% TFA) to afford 40.5 mg (62% yield) of I-55. ESI-MS found 317.2, C₁₃H₂₁N₂O₅S (MH⁺) requires 317.1.

### Synthesis of I-56 is Depicted in Scheme 20

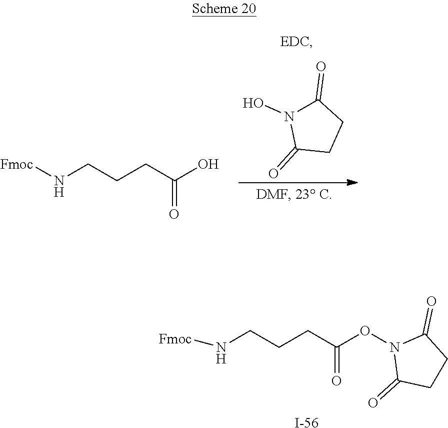

To a solution of N-Fmoc-4-aminobutanoic acid (111.3 mg, 0.34 mmol) in DMF (1 mL) was added HOSu (59.1 mg, 1.5 equiv.) and EDC (98.3 mg, 1.5 equiv.). The reaction mixture was stirred at ambient temperature for 1.5 hours and then purified by preparative HPLC (H₂O/MeCN with 0.1% TFA) to afford 126.2 mg (87% yield) of ester I-56 as a white solid.

### Synthesis of I-57 is Depicted in Scheme 21

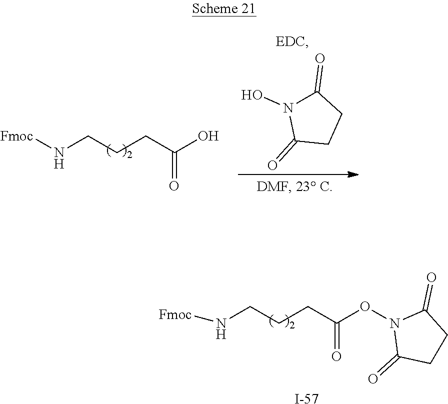

To a solution of N-Fmoc-4-aminopentanoic acid (106.4 mg, 0.31 mmol) in DMF (1 mL) was added HOSu (54.1 mg, 1.5 equiv.) and EDC (90.1 mg, 1.5 equiv.). The reaction mixture was stirred at ambient temperature for 4 hours, then purified by preparative HPLC (H₂O/MeCN with 0.1% TFA) to afford 131.2 mg (96% yield) of ester I-57 as a white solid.

### Synthesis of I-60 is Depicted in Scheme 22

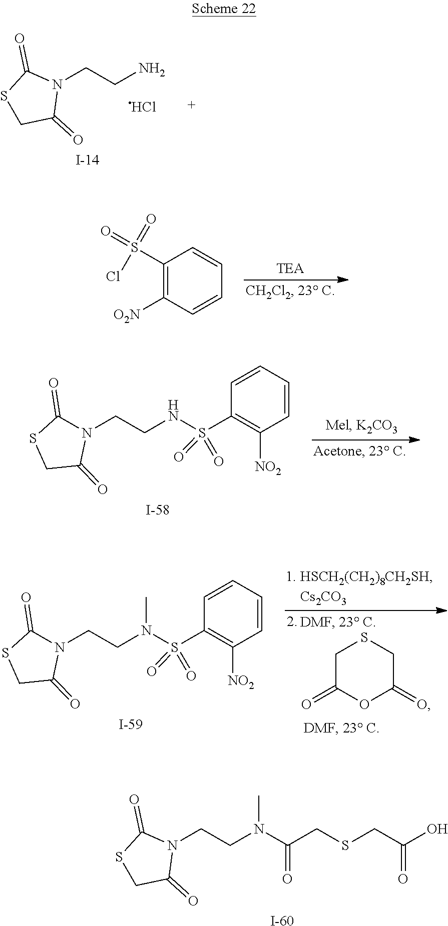

To a solution of amine salt I-14 (202.3 mg, 1.03 mmol) and TEA (0.43 mL, 3.0 equiv.) in DCM (5 mL) was added 2-nitrobenzenesulfonyl chloride (273.6 mg, 1.2 equiv.). The reaction mixture was stirred at ambient temperature for 3 hours, then concentrated and the residue purified by silica gel chromatography (40% EtOAc in hexanes) to afford sulfonamide I-58 (217.7 mg, 61% yield) as a pale yellow solid.

Sulfonamide I-58 (79.7 mg, 0.23 mmol) was dissolved in acetone (3 mL). Potassium carbonate (65.5 mg, 2.0 equiv.) was added, followed by methyl iodide (29 μL, 2.0 equiv.). The reaction mixture was stirred at ambient temperature for 1 hour, then carefully acidified by addition of 1M HCl (5 mL) and extracted with EtOAc (2×7 mL). The combined organic extracts were dried (MgSO₄) and concentrated to a residue, which was purified by preparative TLC (5:1 Toluene/MeCN, 3 elutions) to afford N-methylsulfonamide I-59 (47.7 mg, 58% yield) as a colorless oil.

To N-methylsulfonamide I-59 (47.7 mg, 0.13 mmol) in DMF (1.6 mL) was added 1,10-decanedithiol (41.1 mg, 1.5 equiv.) and cesium carbonate (64.8 mg, 1.5 equiv.). The reaction mixture was stirred at ambient temperature for 4 hours and then filtered. Thiodiglycolic anhydride (26.3 mg, 1.5 equiv.) was added to the filtrate and the reaction mixture maintained at ambient temperature for 2 hours and then diluted with H₂O (0.5 mL). The slurry was clarified by centrifugation and the supernatant was purified directly by preparative HPLC (H₂O/MeCN with 0.1% TFA) to afford 14.4 mg (35% yield) of I-60. ESI-MS found 307.1, C₁₀H₁₅N₂O₅S₂ (MH⁺) requires 307.0.

### Synthesis of I-61 is Depicted in Scheme 23

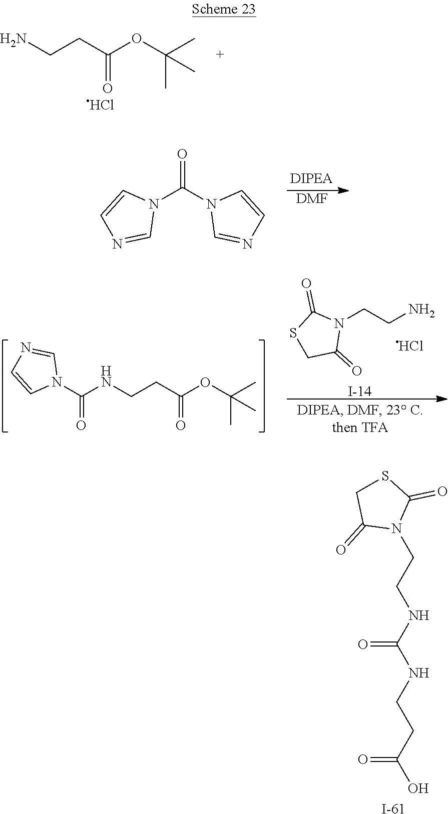

To a solution of beta-alanine-tert-butyl ester hydrochloride (221.3 mg, 1.22 mmol) and CDI (197.5 mg, 1.0 equiv.) in DMF (3 mL) was added DIPEA (1.1 mL, 5.0 equiv). The reaction mixture was stirred for 2 hours, and amine hydrochloride I-14 (239.5 mg 1.0 equiv.) was added. The reaction mixture was heated to 40° C. for 18 hours, and then concentrated under reduced pressure. TFA (5 mL) was added to the residue. The reaction mixture was maintained at ambient temperature for 2 hours, then concentrated under reduced pressure, and purified by preparative HPLC (H₂O/MeCN with 0.1% TFA) to afford 17 mg (5% yield) of I-61 as a colorless oil. ESI-MS found 276.3, C₉H₁₄N₃O₅S (MH⁺) requires 276.1.

### Synthesis of I-62 is Depicted in Scheme 24

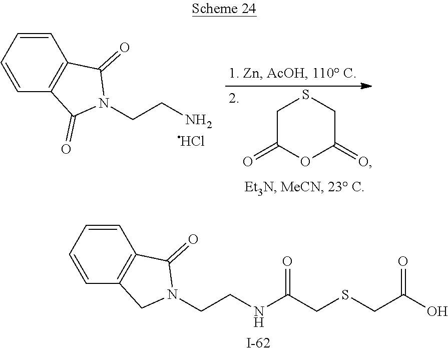

2-(2-Aminoethyl)isoindoline-1,3-dione hydrochloride (316.1 mg, 1.395 mmol) was dissolved in glacial AcOH (7 mL). Zinc dust (912.2 mg, 10 equiv.) was added, and the reaction mixture was heated to 110° C. for 18.5 hours, filtered hot, and concentrated under reduced pressure to afford the crude reduction product (900 mg). Half of this material was dissolved in MeCN (5 mL). Thiodiglycolic anhydride (120 mg, 0.91 mmol) was added, followed by triethylamine (486 μL, 5 equiv.). The reaction mixture was stirred at ambient temperature for 16 hours, concentrated, and purified by preparative HPLC (H₂O/MeCN with 0.1% TFA) to afford 20.4 mg (10% yield) of I-62 as a colorless oil. ESI-MS found 309.2, C₁₄H₁₇N₂O₄S (MH⁺) requires 309.1.

### Synthesis of I-64 is Depicted in Scheme 25

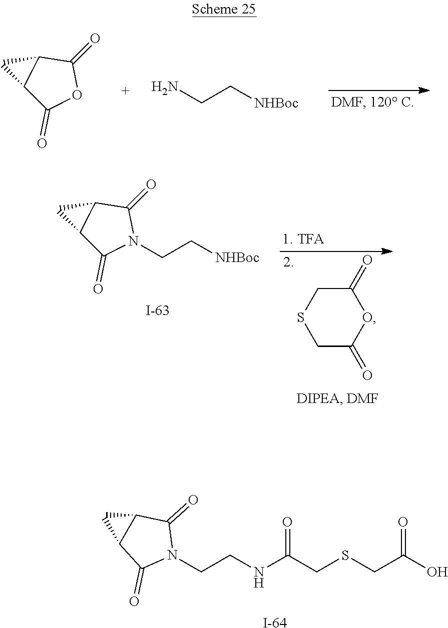

To a solution of (1R,5S)-3-oxabicyclo[3.1.0]hexane-2,4-dione (168.3 mg, 1.50 mmol) in DMF (5 mL) was added N-tert-butyloxycarbonyl ethylenediamine (360.8 mg 1.5 equiv.). The reaction mixture was stirred at ambient temperature for 30 minutes, and then heated at 120° C. for 4.5 hours. The reaction mixture was then cooled and diluted with EtOAc (50 mL). The solution was washed with saturated aqueous NH₄Cl (20 mL) and water (3×20 mL). The combined washes were back-extracted with EtOAc (30 mL). The combined organic extracts were washed with brine (20 mL), dried (MgSO₄), and concentrated to afford a residue, which was purified by silica gel chromatography (0-60% EtOAc in hexanes) to afford 174.3 mg (46% yield) of carbamate I-63 as a white solid.

To 34.8 mg (0.137 mmol) of carbamate I-63 was added TFA (1 mL). The reaction mixture was maintained at ambient temperature for 30 minutes and then concentrated. The residue was re-dissolved in DMF (0.8 mL), and DIPEA (119.2 μL, 5.0 equiv.) was added, followed by thiodiglycolic anhydride (21.7 mg, 1.2 equiv.). The reaction mixture was maintained at ambient temperature for 2 hours and then purified by preparative HPLC (H₂O/MeCN with 0.1% TFA) to afford 31.6 mg (81% yield) of I-64. ESI-MS found 287.0, C₁₁H₁₅N₂O₅S (MH⁺) requires 287.1.

### Synthesis of I-66 is Depicted in Scheme 26

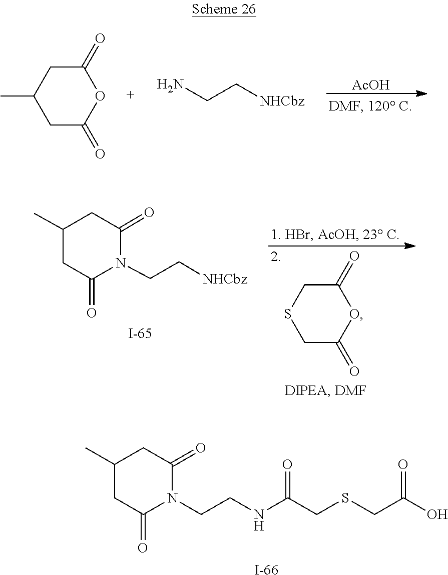

To a solution of 3-methylglutaric anhydride (247 mg, 1.93 mmol) in DMF (1 mL) was added N-benzyloxycarbonyl ethylenediamine (374.4 mg, 1.0 equiv.). The reaction mixture was stirred at ambient temperature for 1.5 hours. AcOH (2 mL) was then added. The reaction mixture was heated to 110° C. for 21.5 hours and then cooled to ambient temperature. The reaction mixture was diluted with water (10 mL) and extracted with EtOAc (3×5 mL). The combined extracts were dried (MgSO₄) and concentrated to a residue. Purification by silica gel chromatography (0-63% EtOAc in hexanes) afforded carbamate I-65 (432.8 mg, 74% yield) as a white solid.

A solution of HBr in AcOH (33% v/v, 3 mL) was added to carbamate I-65 (104 mg, 0.34 mmol). The reaction mixture was sonicated for 5 minutes to solubilize the solids and then maintained at ambient temperature for 45 minutes, after which it was concentrated under reduced pressure. The residue was triturated with Et₂O (10 mL) and the supernatant was discarded. The residue was dissolved in DMF (1.2 mL). DIPEA (179 μL, 3.0 equiv.) was added, followed by thiodiglycolic anhydride (49.7 mg, 1.1 equiv.). The reaction mixture was maintained at ambient temperature for 1.5 hours and then purified by preparative HPLC (H₂O/MeCN with 0.1% TFA) to afford 69 mg (67% yield) of I-66. ESI-MS found 303.3, C₁₂H₁₈N₂O₅S (MH⁺) requires 303.1.

### Synthesis of (±)-I-67 is Depicted in Scheme 27

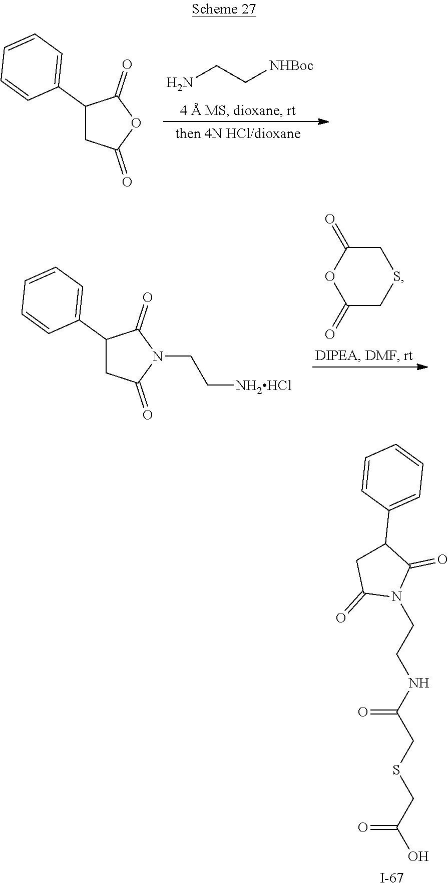

A solution of 3-phenyldihydrofuran-2,5-dione (71.2 mg, 0.40 mmol) and (2-aminoethyl)carbamate (64.1 mg, 1.0 equiv.) in 1 mL of anhydrous dioxane was stirred over 4 Å molecular sieves for 5.5 hours at ambient temperature. The reaction mixture was filtered and concentrated under reduced pressure. The residue obtained was dissolved in 4N HCl in dioxane (3 mL). After stirring at ambient temperature for 16 hours, the reaction mixture was concentrated under reduced pressure to afford the crude amine hydrochloride. Amine hydrochloride (39 mg, 0.153 mmol) was converted to 17.6 mg of acid I-67 (33% yield) following GP1. ESI-MS: Found 348.9, C₁₆H₁₈N₂O₅S (MH⁻) requires 349.1.

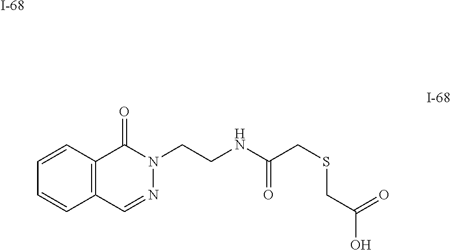

Prepared according to GP1. Yield: 68.1 mg (97%). ESI-MS found 322.2, C₁₄H₁₆N₃O₄S (MH⁺) requires 322.1.

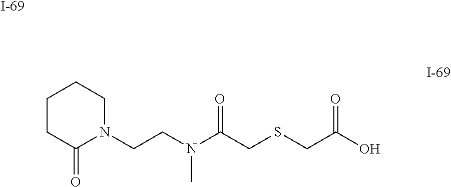

Prepared according to GP1. Yield: 48.1 mg (55%). ESI-MS found 289.2, C₁₅H₁₉N₂O₄S (MH⁺) requires 289.1.

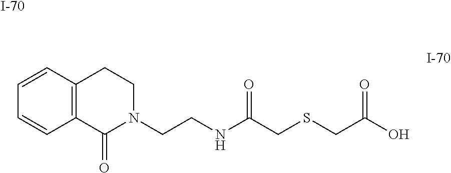

Prepared according to GP1. Yield: 37.7 mg (73%). ESI-MS found 323.2, C₁₂H₂₁N₂O₄S (MH⁺) requires 323.1.

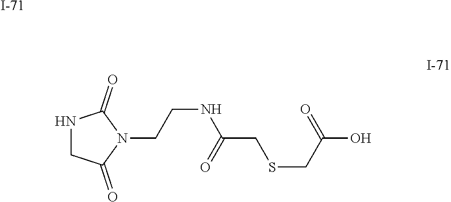

Prepared according to GP1. Yield: 9.9 mg (20%). ESI-MS found 276.1, C₉H₁₄N₃O₅S (MH⁺) requires 276.1.

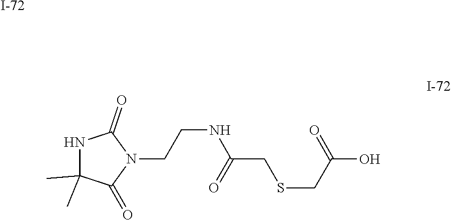

Prepared according to GP1. Yield: 60 mg (88%). ESI-MS found 304.2, C₁₁H₁₈N₃O₅S (MH⁺) requires 304.1.

### Synthesis of (±)-I-74 is Depicted in Scheme 28

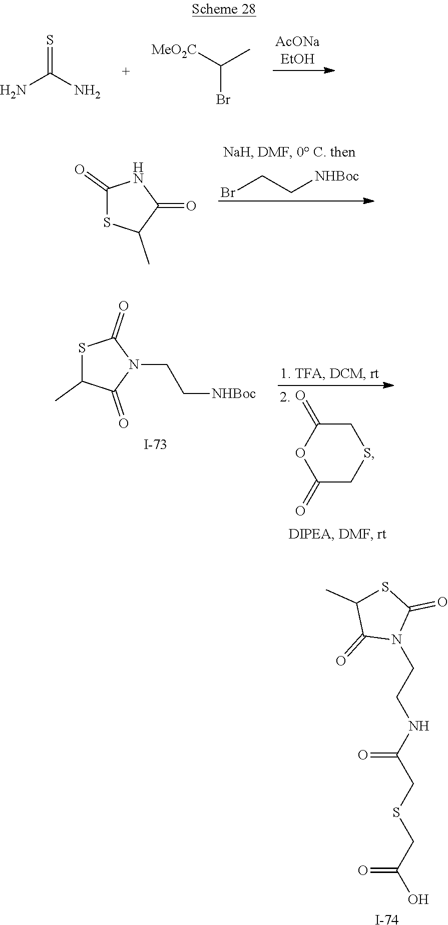

To a solution of methyl 2-bromopropanoate (200 μL, 1.79 mmol) in THF (9 mL) were added thiourea (177 mg, 1.3 equiv.) and sodium acetate (293 mg, 2.0 equiv.). The reaction mixture was heated under reflux for 20 hours. 6N HCl (4 mL) was then added. The reaction mixture was heated under reflux for an additional 16 hours and then allowed to cool to ambient temperature. Water was then added, and the reaction mixture was extracted with EtOAc. The combined organic extracts were washed with brine, dried (sodium sulfate), and concentrated. The residue obtained was subjected to silica gel chromatography (0 to 7% MeOH in DCM) to afford 235 mg of 5-methylthiazolidine-2,4-dione as a clear oil (>95% yield).

To a solution of 5-methylthiazolidine-2,4-dione (112 mg, 0.854 mmol) in DMF (6 mL) at 0° C. was added sodium hydride (41 mg of a 60% dispersion in mineral oil, 1.2 equiv.), and the resulting slurry was stirred for 30 minutes. A solution of tert-butyl (2-bromoethyl)carbamate (304 mg, 1.6 equiv.) in DMF (1 mL) was then added and the reaction mixture was allowed to warm to ambient temperature and stirred for two hours. The reaction was quenched with 45 mL of saturated NH₄Cl solution and then extracted three times with EtOAc. The combined organic extracts were washed with brine, dried (Na₂SO₄), and concentrated in vacuo to afford a residue, which was purified by silica gel chromatography to afford 202 mg (86% yield) of carbamate I-73. Deprotection of carbamate I-73 in TFA/DCM (1:1 v/v) for 2 hours at ambient temperature was followed by concentration of the reaction mixture in vacuo to give a crude trifluoroacetate salt, which was converted to 99.9 mg of acid I-74 (44% yield) following GP1. ESI-MS: Found 305.1, C₁₀H₁₃N₂O₅S₂ (M−H)⁻ requires 305.0.

### Synthesis of I-74 is Depicted in Scheme 29

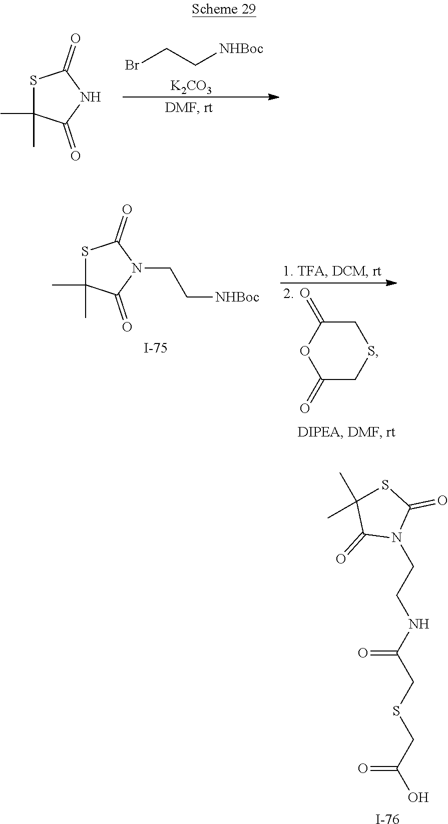

To a solution of 5,5-dimethylthiazolidine-2,4-dione (163 mg, 1.12 mmol) and tert-butyl (2-bromoethyl)carbamate (377 mg, 1.5 equiv.) in 5 mL of anhydrous DMF was added potassium carbonate (309 mg, 2.0 equiv.), and the resulting suspension was stirred for 18 hours at ambient temperature. The reaction mixture was diluted with 50 mL of EtOAc. The organic phase was washed twice with water, brine, dried over sodium sulfate, and concentrated in vacuo to afford a residue, which was purified by silica gel chromatography to afford 275 mg (83% yield) of carbamate I-75. Deprotection of carbamate I-73 in TFA/DCM (1:1 v/v) for two hours at ambient temperature was followed by concentration of the reaction mixture in vacuo to give a crude trifluoroacetate salt. This amine TFA salt (66 mg, ca. 0.218 mmol) was converted to 36 mg of acid I-76 (52% yield) following GP1. ESI-MS: Found 319.0, C₁₁H₁₅N₂O₅S₂ (M−H)⁻ requires 319.1.

### Synthesis of I-79 and I-80 is depicted in Scheme 30

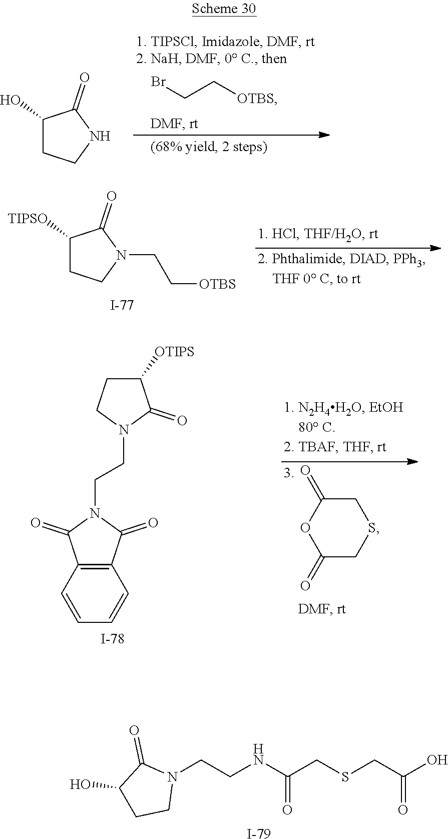

I-79: to a solution of (S)-3-hydroxypyrrolidinone (488 mg, 4.83 mmol) in DMF (10 mL), was added imidazole (493 mg, 1.5 equiv.), followed by triisopropylsilyl chloride (1.12 g, 1.2 equiv.). The reaction mixture was stirred at ambient temperature. On completion of the reaction, the reaction mixture was diluted with EtOAc (100 mL) and washed with H₂O (5×20 mL). The aqueous washes were combined and back-extracted with EtOAc (50 mL). The combined organic extracts were washed with brine (50 mL), dried (MgSO₄) and concentrated to afford a colorless oil. Purification by silica gel chromatography (12 g pre-packed column, eluting with 35-60% EtOAc in Hexanes) afforded 1.23 g (99% yield) of the TIPS-protected alcohol as a colorless oil.

The silyl ether was then dissolved in DMF (7 mL). This solution was added to a suspension of NaH (574 mg of 60% dispersion in mineral oil, 3.0 equiv.) in DMF (15 mL) that had been pre-cooled to 0-5° C. After 4 minutes, (2-bromoethoxy)(tert-butyl)dimethylsilane (1.37 g, 1.2 equiv.) was added. The reaction mixture was allowed to warm to ambient temperature. After 1.5 hours, the reaction mixture was quenched with saturated aqueous NH₄Cl (20 mL), and diluted with brine (100 mL). The aqueous phase was extracted with EtOAc (3×70 mL). The combined EtOAc extracts were dried (MgSO₄) and concentrated to afford a colorless oil. Purification by silica gel chromatography (24 g pre-packed column, eluting with 30% EtOAc in Hexanes) afforded 1.38 g (69% yield) of the bis-silyl ether I-77 as a colorless oil. This bis-silyl ether was dissolved in THF (12 mL). IN HCl (4 mL) was then added. The reaction mixture was stirred at ambient temperature for 2 hours, then diluted with H₂O (100 mL) and extracted with EtOAc (3×60 mL). The combined organic extracts were dried (Na₂SO₄) and concentrated to afford a colorless oil. Purification by silica gel chromatography (24 g pre-packed column, eluting with 80-100% EtOAc in Hexanes) afforded 0.85 g (85% yield) of the deprotection product. This deprotected silyl ether was dissolved in THF (20 mL) in the presence of triphenylphosphine (1.11 g, 1.5 equiv.) and phthalimide (622 mg, 1.5 equiv.). The solution was cooled to 0-5° C., and DIAD (833 μL, 1.5 equiv.) was added. After 1.5 hours, the reaction mixture was concentrated to a yellow oil, which was purified by silica gel chromatography (40 g pre-packed column, eluting with 44% EtOAc in Hexanes) to afford 2.3 g of phthalimide I-78 (˜50% pure, with diisopropylhydrazine-1,2-dicarboxylate as impurity).

To a solution of phthalimide I-78 (254 mg, 0.59 mmol, ˜50% pure, with diisopropylhydrazine-1,2-dicarboxylate as impurity) in EtOH (3 mL) was added hydrazine hydrate (183 μL, 10.0 equiv.). The reaction mixture was heated to 80° C. for 20 minutes, cooled and concentrated. The residue was re-suspended in MeOH (5 mL) and filtered. The filtrate was concentrated to a residue and TBAF (1 Min THF, 1.2 mL, 2.0 equiv. based on phthalimide I-78) was added. The reaction mixture was stirred at ambient temperature for 19 hours, then concentrated. The residue was re-dissolved in DMF (2 mL) and thiodiglycolic anhydride (78 mg, 1.0 equiv. based on phthalimide I-78) was added. After 2 hours, the reaction mixture was subjected to purification by preparative HPLC (H₂O/MeCN with 0.1% TFA) to afford 10.6 mg of the desired carboxylic acid I-79 as a colorless oil. ESI-MS: Found 277.2, C₁₀H₁₇N₂O₅S (MH⁺) requires 277.1.

I-80: Analogous procedure was used for the preparation of the enantiomer, I-80. ESI-MS: Found 277.2, C₁₀H₁₇N₂O₅S (MH⁺) requires 277.1.

### Synthesis of I-82 is Depicted in Scheme 31

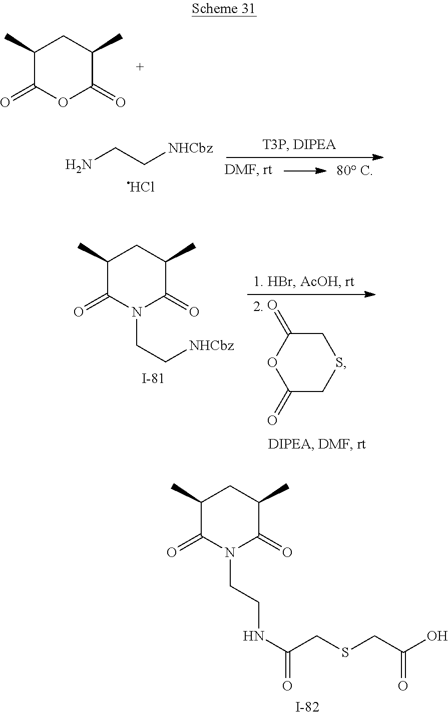

A slurry of (3R,5S)-3,5-dimethyldihydro-2H-pyran-2,6(3H)-dione (102.8 mg, 0.723 mmol), benzyl(2-aminoethyl)carbamate hydrochloride (166.8 mg, 1.0 equiv.), T3P (50% w/v solution in DMF, 0.92 mL, 2.0 equiv.) and DIPEA (378 μL, 3.0 equiv.) in DMF (2 mL) was stirred at ambient temperature for 1 hour, then heated to 80° C. for 2 hours. The reaction mixture was cooled to ambient temperature, diluted with brine (10 mL), and then extracted with EtOAc (3×15 mL). The combined organic extracts were dried (MgSO₄) and concentrated to afford an oil, which was purified by silica gel chromatography (12 g pre-packed column, eluting at 51% EtOAc/Hexanes) to afford imide I-81 (77 mg, 33% yield) as a colorless oil.

To imide I-81 (77 mg, 0.24 mmol) was added 33% v/v HBr/AcOH (3 mL). The reaction mixture was maintained at ambient temperature for 1 hour and then concentrated. The residue was triturated with Et₂O (10 mL) and the Et₂O was discarded. The remaining residue was dissolved in DMF (1.5 mL). Thiodiglycolic anhydride (32 mg, 1.0 equiv. based on imide I-81) was added, followed by DIPEA (126 μL, 3.0 equiv.). The reaction mixture was maintained at ambient temperature for 20 hours and then purified by preparative HPLC (H₂O/MeCN with 0.1% TFA) to afford 59.4 mg (78% yield) of I-82 as a colorless oil. ESI-MS: Found 317.3, C₁₃H₂₁N₂O₅S (MH⁺) requires 317.1.

### Synthesis of I-85 is Depicted in Scheme 32

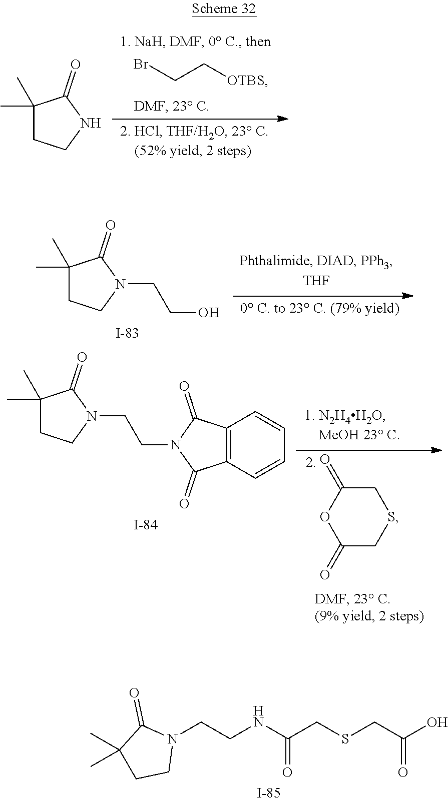

To a slurry of 447.7 mg (5.0 equiv.) of sodium hydride (60% dispersion in mineral oil) in DMF (5 mL) that had been cooled to 0-5° C. was added a solution of 2,2-dimethylpyrollidinone (253.3 mg, 2.238 mmol) in DMF (7 mL). The reaction mixture was stirred at 0-5° C. for 10 minutes and (2-bromoethoxy)(tert-butyl)dimethylsilane (642.5 mg, 1.2 equiv.) was added in one portion. The reaction mixture was stirred for 10 minutes at 0-5° C., then allowed to warm to ambient temperature. After 18 hours, the reaction mixture was cooled to 0-5° C., and the reaction was quenched with saturated aqueous NH₄Cl (10 mL). The reaction mixture was diluted with 50% brine (100 mL) and extracted with EtOAc (3×50 mL). The organic extracts were combined, dried (Na₂SO₄), and concentrated to afford a colorless oil which was then dissolved in THF (12 mL). Aqueous HCl (1M, 4 mL) was added, and the reaction mixture was stirred at ambient temperature for 5 hours. The reaction was quenched with saturated aqueous NaHCO₃ (10 mL) and diluted with brine (50 mL), then extracted with EtOAc (3×50 mL). The organic extracts were combined, dried (Na₂SO₄) and concentrated to afford a colorless oil, which was purified by silica gel chromatography (Hexanes/Acetone 0-100% gradient) to afford intermediate alcohol I-83 (183.8 mg, 52% yield over 2 steps) as a colorless oil.

To a solution of alcohol I-83 (183.8 mg, 1.17 mmol), phthalimide (258 mg, 1.5 equiv.) and triphenylphosphine (460 mg, 1.5 equiv.) in THF (6 mL) that had been cooled to 0-5° C. was added DIAD (0.35 mL, 1.5 equiv.). The reaction mixture was allowed to warm to ambient temperature and concentrated after 2.5 hours. Purification of the resulting residue by silica gel chromatography (EtOAc) afforded phthalimide I-84 (263.2 mg 79% yield) as a colorless oil.

To a solution of phthalimide I-84 (263.2 mg, 0.92 mmol) in MeOH (5 mL) was added hydrazine hydrate (0.29 mL, 10.0 Equiv). The reaction mixture was stirred at ambient temperature for 24 hours and then filtered. The filter cake was washed with MeOH (10 mL). The combined filtrate and wash were concentrated to a residue, which was re-dissolved in DMF (3 mL). Thiodiglycolic anhydride (121.5 mg, 1.0 equiv.) was added and the reaction mixture was stirred at ambient temperature for 4 hours, then purified by preparative HPLC (0-30% MeCN/25 mM NH₄OAc gradient over 35 minutes) to afford 22.8 mg (9% yield, 2 steps) of I-85 as a colorless oil. ESI-MS: Found 289.2, C₁₂H₂₁N₂O₄S (MH⁺) requires 289.1.

### Synthesis of I-87 is Depicted in Scheme 33

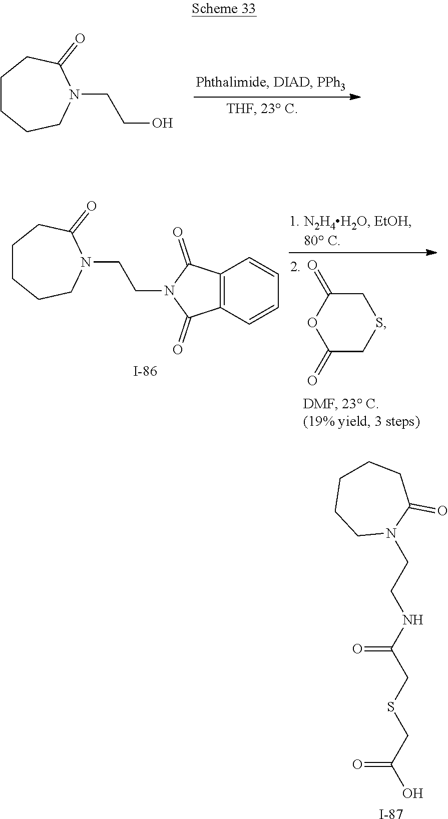

To a solution of 1-(2-hydroxyethyl) azepan-2-one (97 mg, 0.62 mmol), triphenylphosphine (242.9 mg, 1.5 equiv.), and phthalimide (136.1 mg, 1.5 equiv.) in THF (3 mL) at ambient temperature was added DIAD (0.18 mL, 1.5 equiv.). The reaction mixture was allowed to warm to ambient temperature and concentrated after 2.5 hours. Purification of the resulting residue by silica gel chromatography (EtOAc) afforded phthalimide I-86, which co-eluted with triphenylphosphine oxide. The phthalimide was dissolved in EtOH (10 mL) and hydrazine hydrate (0.19 mL, 10 equiv.) was added. The reaction mixture was heated to 80° C. for 1.5 hours and then cooled to ambient temperature and filtered. The filtrate was concentrated to afford a residue. DMF (3 mL) was added, and the suspension afforded was clarified by centrifugation. Thiodiglycolic anhydride (81.5 mg, 1.0 equiv.) was added to the supernatant and the reaction mixture was maintained at ambient temperature for 24 hours. Purification by preparative HPLC (H₂O/MeCN with 0.1% TFA) to afford 34.1 mg (19% yield, 3 steps) of I-87 as a colorless oil. ESI-MS: Found 289.2, C₁₂H₂₁N₂O₄S (MH⁺) requires 289.1.

### Synthesis of I-89 and I-90 is Depicted in Scheme 34

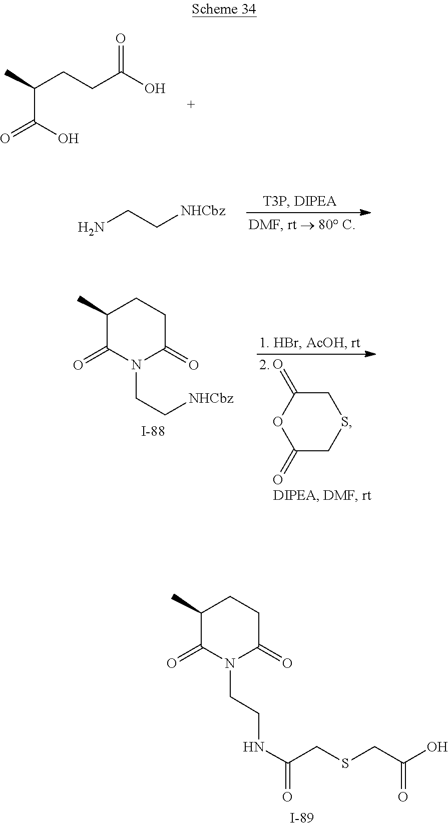

I-89: a slurry of (S)-2-methylglutaric acid (68.5 mg, 0.469 mmol), benzyl(2-aminoethyl)carbamate (91.1 mg, 1.0 equiv.), T3P (50% w/v solution in DMF, 1.5 mL, 5.0 equiv.), and DIPEA (817 μL, 10.0 equiv.) in DMF (3 mL) was stirred at ambient temperature for 63 hours and then heated to 80° C. for 4 hours. The reaction mixture was cooled and diluted with EtOAc (10 mL) and saturated aqueous NaHCO₃ (10 mL). The phases were separated, and the aqueous phase was extracted with EtOAc (2×10 mL). The combined organic extracts were dried (MgSO₄) and concentrated to afford a colorless oil, which was purified by silica gel chromatography to afford imide 1-88 (25.8 mg, 18% yield) as a colorless oil.

To imide I-88 (25.8 mg, 0.085 mmol) was added 33% v/v HBr/AcOH (2 mL). The reaction mixture was maintained at ambient temperature for 1 hour and then concentrated. The residue was triturated with Et₂O (10 mL) and the Et₂O was discarded. The remaining residue was dissolved in DMF (1 mL). Thiodiglycolic anhydride (13.4 mg, 1.2 equiv. based on imide I-88) was added, followed by DIPEA (74 μL, 5.0 equiv.). The reaction mixture was maintained at ambient temperature for 16 hours and then purified by preparative HPLC (H₂O/MeCN with 0.1% TFA) to afford 20.6 mg (81% yield) of I-89 as a colorless oil. ESI-MS: Found 303.2, C₁₂H₁₉N₂O₅S (MH⁺) requires 303.1.

I-90: an analogous procedure was used to prepare enantiomeric building block I-90. Yield: 43 mg (62%). ESI-MS: Found 303.2, C₁₂H₁₉N₂O₅S (MH⁺) requires 303.1.

### Synthesis of I-92 is Depicted in Scheme 35

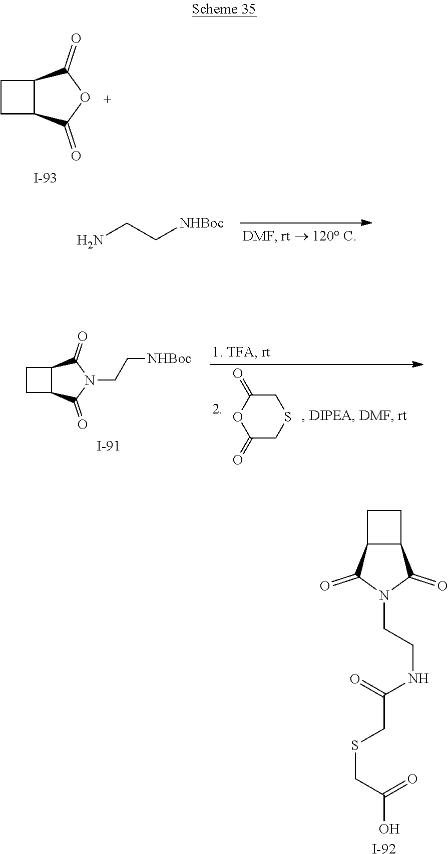

To a solution of anhydride I-93 (106.8 mg, 0.85 mmol) in DMF (2 mL) was added tert-butyl (2-aminoethyl)carbamate (203.5 mg, 1.5 equiv.). The reaction mixture was maintained at ambient temperature for 1.5 hours and then heated to 120° C. for 6 hours and 90° C. for 60 hours. The reaction mixture was cooled, diluted with H₂O (10 mL), and then extracted with EtOAc (3×6 mL). The combined organic extracts were dried (MgSO₄) and concentrated to afford a brown solid, which was purified by silica gel chromatography (12 g pre-packed column, 63% EtOAc/Hexanes) to afford imide I-91 (165.3 mg, 73% yield) as a colorless oil.

To imide I-91 (43.3 mg (0.161 mmol) was added TFA (2 mL). After 15 minutes at ambient temperature the reaction mixture was concentrated to a residue, which was redissolved in DMF (1 mL). Thiodiglycolic anhydride (23.5 mg, 1.1 equiv. based on imide I-91) was added, followed by DIPEA (84 μL, 3.0 equiv.). The reaction mixture was maintained at ambient temperature for 24 hours and then purified by preparative HPLC (H₂O/MeCN with 0.1% TFA) to afford 42.4 mg (87% yield) of I-92 as a colorless oil. ESI-MS: Found 301.2, C₁₂H₁₇N₂O₅S (MH⁺) requires 301.1.

### Synthesis of I-94 is depicted in Scheme 36

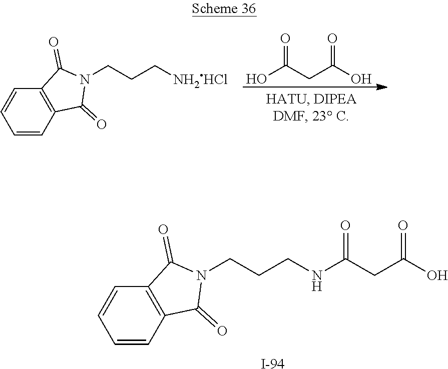

To a solution of 2-(3-aminopropyl) isoindoline-1,3-dione hydrochloride (49.8 mg 0.207 mmol), malonic acid (43.1 mg, 2.0 equiv.), and DIPEA (0.11 mL, 3.0 equiv.) in DMF (0.9 mL) was added HATU (157.3 mg, 2.0 equiv.). The reaction mixture was agitated at ambient temperature for 3 hours and then purified by preparative HPLC (H₂O/MeCN with 0.1% TFA) to afford 6.7 mg (11% yield) of I-94 as a colorless oil. ESI-MS: Found 291.1, C₁₄H₁₅N₂O₅ (MH⁺) requires 291.1.

### Synthesis of I-96 is Depicted in Scheme 37

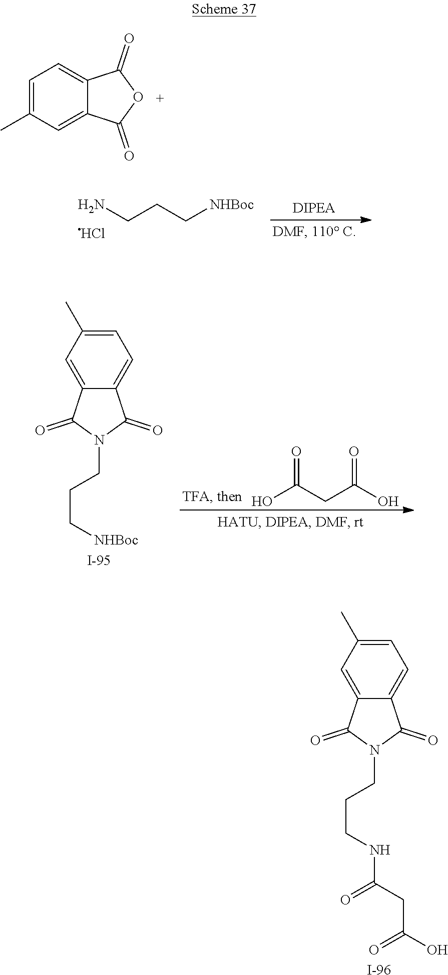

5-methylisobenzofuran-1,3-dione (305 mg, 1.88 mmol) was dissolved in DMF (5 mL). Tert-butyl (3-aminopropyl) carbamate hydrochloride (595 mg, 1.5 equiv.) was added, followed by DIPEA (983 μL, 3.0 equiv.). The reaction mixture was heated to 110° C. for 18 hours. The reaction mixture was then cooled and diluted with EtOAc (50 mL). The EtOAc solution was washed with saturated aq. NH₄Cl (20 mL) and H₂O (3×20 mL). The aqueous washes were back-extracted with EtOAc (30 mL). The combined organic extracts were washed with brine (20 mL), dried (MgSO₄), and concentrated to a solid which was purified by silica gel chromatography (39% EtOAc/Hexanes) to afford 481 mg (80% yield) of intermediate carbamate I-95 as a white solid.

To carbamate I-95 (40.6 mg, 0.128 mmol) was added TFA (1 mL). After 30 minutes at ambient temperature, the TFA was removed in vacuo. Malonic acid (26.5 mg 2.0 equiv.) was added, followed by DIPEA (67 μL, 3.0 equiv.) and HATU (97 mg, 2.0 equiv.) The reaction mixture was stirred at ambient temperature for 1 hour, then purified directly by HPLC (H₂O/MeCN with 0.1% TFA) to afford 13.1 mg (34% yield) of I-96. ESI-MS found 305.3, C₁₅H₁₇N₂O₅ (MH⁺) requires 305.1.

### Synthesis of I-98 is Depicted in Scheme 38

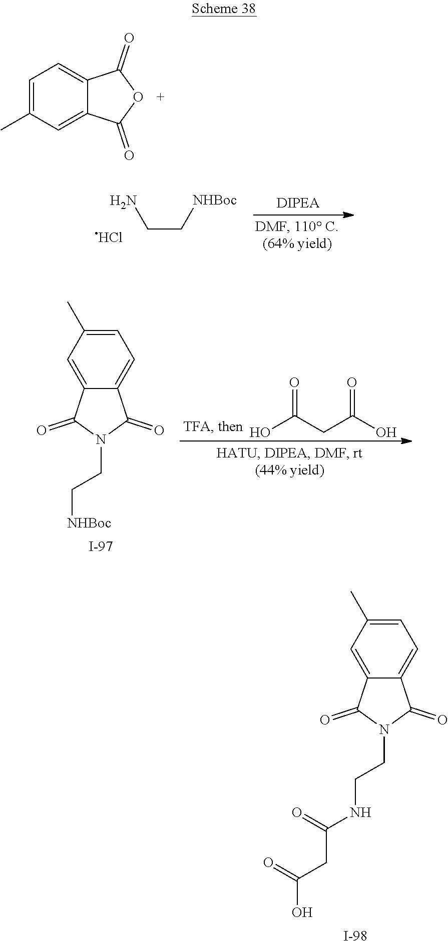

Using a procedure similar to that described for the preparation of I-96, 295 mg (1.82 mmol) of 5-methylisobenzofuran-1,3-dione afforded 356.7 mg (64% yield) of phthalimide I-97. Phthalimide I-97 (44.2 mg, 0.145 mmol) then afforded 18.5 mg (44% yield, 2 steps) of I-98. ESI-MS found 291.2, C₁₄H₁₅N₂O₅ (MH⁺) requires 291.1.

### Synthesis of I-100 is Depicted in Scheme 39

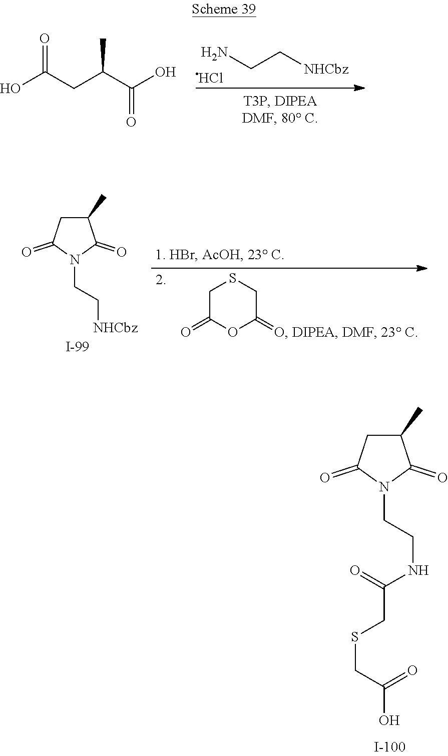

To (R)-2-methylsuccinic acid (174.5 mg, 1.32 mmol) in DMF (2 mL) was added benzyl(2-aminoethyl)carbamate hydrochloride (304.7 mg, 1.0 equiv.), DIPEA (0.69 mL, 3.0 equiv.), and propylphosphonic anhydride (50% in DMF, 2.5 mL, 3.0 equiv.). The reaction mixture was heated to 80° C. for 1.5 hours, then cooled and concentrated. The resulting residue was purified by silica gel chromatography to afford 48 mg (13%) of the intermediate succinimide I-99. HBr (33% solution in AcOH, 2 mL) was added to I-99. The reaction mixture was maintained at ambient temperature for 45 minutes, then concentrated under reduced pressure. The residue afforded was triturated with Et₂O (10 mL) and then redissolved in DMF (0.7 mL). DIPEA (86 μL, 3.0 equiv) was added, followed by thiodiglycolic anhydride (21.8 mg, 1.0 equiv.). The reaction mixture was maintained at ambient temperature for 3 hours and then purified by preparative HPLC (0-100% acetonitrile in water with 0.1% TFA) to afford 15.3 mg (32% yield) of I-100. ESI-MS found 289.2, C₁₁H₁₇N₂O₅S (MH⁺) requires 289.1.

I-101: an analogous procedure was used to synthesize enantiomeric building block I-101. Yield 16.2 mg (6% yield, 3 steps). ESI-MS found 289.2, C₁₁H₁₇N₂O₅S (MH⁺) requires 289.1.

### Synthesis of I-104 is Depicted in Scheme 40

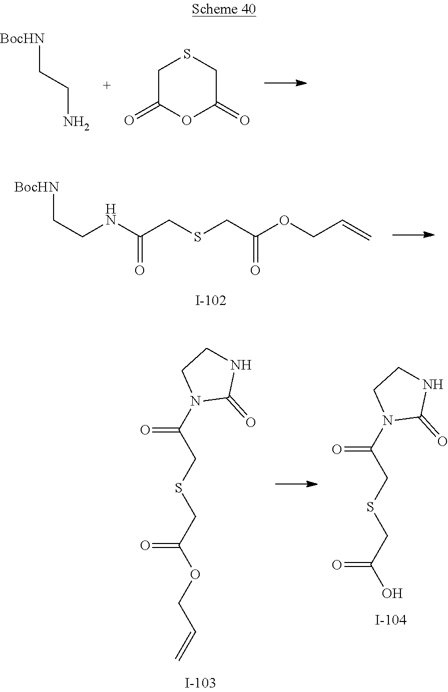 [SMILES: CC(C)(C)OC(=O)NCCN ; O=C1CSCC(=O)O1 ; C=CCOC(=O)CSCC(=O)NCCNC(=O)OC(C)(C)C ; C=CCOC(=O)CSCC(=O)N1CCNC1=O ; O=C(O)CSCC(=O)N1CCNC1=O]

A solution of tert-butyl (2-aminoethyl)carbamate (415 mg, 2.6 mmol) and thiodiglycolic anhydride (376 mg, 1.1 equiv.) in 5 mL of DMF was treated with TEA (1.27 mL, 1.4 equiv.). The reaction mixture was stirred for 3 hours at ambient temperature at which time allyl bromide (336 μL, 1.5 equiv.) was added and the reaction mixture was stirred for an additional 72 hours. The reaction mixture was then diluted with EtOAc (40 mL). The organic phase was washed with saturated aqueous NaHCO₃, brine, dried (Na₂SO₄), and concentrated in vacuo. Purification by flash chromatography (0% to 70% EtOAc in hexanes) afforded 250 mg of I-102 as a clear oil (75% yield). ESI-MS found 331.2, C₁₄H₂₃N₂O₅S (M−H)⁻ requires 331.1

A solution of I-102 (135 mg, 0.406 mmol) in 1 mL of dioxane was treated with 3 mL of 4N HCl/dioxane and stirred for 2 hours. The reaction mixture was concentrated under reduced pressure and then placed under high vacuum for 8 hours to afford 112 mg of intermediate hydrochloride salt, a portion of which (84.1 mg, 0.313 mmol) was suspended in 5 mL of DCM, treated with DIPEA (223 μL, 4.0 equiv.) and sonicated for 5 minutes. The resulting suspension was filtered through a plug of cotton, treated with proton sponge (201 mg, 3.0 equiv.), and added to a solution of triphosgene (56 mg, 0.6 equiv.) in 5 mL of DCM at 0° C. under N₂. After stirring for 4 hours at 0-5° C., the reaction mixture was allowed to warm to ambient temperature, and stirring was continued another 24 hours. The reaction mixture was then diluted with 45 mL of DCM. The organic phase was washed twice with 1M aqueous NaHSO₄, brine, dried over Na₂SO₄ and concentrated in vacuo. The resulting residue was purified by preparative HPLC (0-100% acetonitrile in water with 0.1% TFA) to afford 32 mg of I-103 as a white solid (31% yield). ESI-MS found 259.2, C₁₀H₁₃N₂O₄S (MH⁺) requires 259.1

A solution of I-103 (42 mg, 0.161 mmol) in 4 mL of DCM was treated with Pd(PPh₃) 4 (46.6 mg, 0.25 equiv.) and phenylsilane (79 μL, 4.0 equiv.) and the reaction mixture was stirred at ambient temperature for one hour. The reaction mixture was then concentrated in vacuo and the residue was suspended in 2.5 mL of 3:1 v/v DMSO/H₂O and filtered to give a clear solution, which was then purified directly by preparative HPLC (0-100% acetonitrile in water with 0.1% TFA) to afford 18.5 mg of I-104 as a white solid (53% yield). ESI-MS found 217.1, C₇H₉N₂O₄S (MH⁻) requires 217.0.

### Synthesis of I-106 is Depicted in Scheme 41

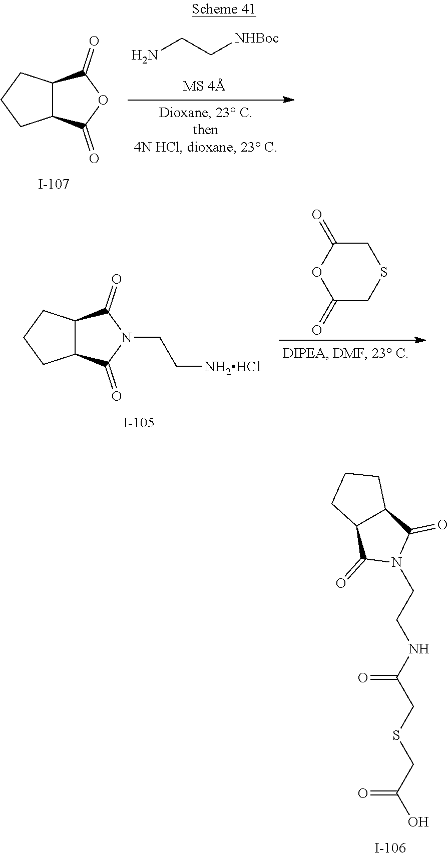

A solution of anhydride I-107 (42 mg, 0.300 mmol) and tert-butyl (2-aminoethyl)carbamate (57.6 mg, 1.2 equiv.) in 3 mL of anhydrous dioxane were stirred over 4 Å molecular sieves for 1 hour and then decanted. 4N hydrogen chloride in dioxane (2 mL) was added to the supernatant and the reaction mixture was stirred for 16 hours and then concentrated in vacuo to afford the crude hydrochloride salt I-105. The crude hydrochloride salt was converted to 27.3 mg of acid I-106 (29% yield over 3 steps) using GP1. ESI-MS found 312.9, C₁₃H₁₈N₂O₅S (MH⁻) requires 313.1.

### Synthesis of I-109 is Depicted in Scheme 42

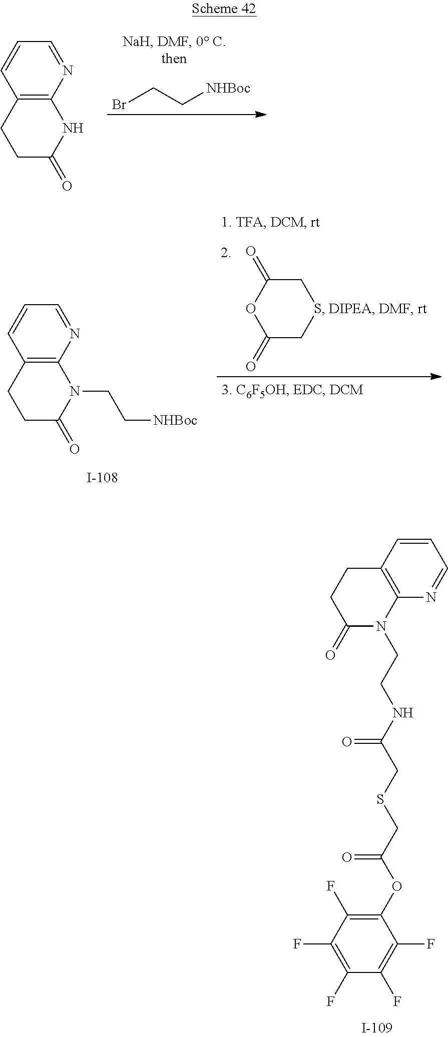

To a solution of 3,4-dihydro-1,8-naphthyridin-2(1H)-one (106 mg, 0.714 mmol) in 2.4 mL of anhydrous DMF at 0° C. was added sodium hydride (34.4 mg of a 60% dispersion in mineral oil, 1.2 equiv.), and the resulting suspension was stirred 10 minutes. Tert-butyl (2-bromoethyl)carbamate (176 mg, 1.1 equiv.) was added in two portions and the solution was allowed to warm to ambient temperature and stirred for two hours. The reaction was quenched with 25 mL of saturated ammonium chloride solution and then extracted three times with EtOAc. The combined organic extracts were washed with brine, dried over sodium sulfate and concentrated in vacuo to afford a residue, which was purified by silica gel chromatography to afford 104 mg (50% yield) of carbamate I-108. Carbamate I-108 (84 mg, 0.287 mmol) was deprotected with TFA/DCM (1:1 v/v) for two hours at ambient temperature. The reaction mixture was concentrated under reduced pressure to afford a crude trifluoroacetate salt, which was dissolved in 1 mL of DMF and 5 mL of DCM and treated with a solution of thiodiglycolic anhydride (37 mg, 1.0 equiv. based on carbamate I-108) and DIPEA (200 μL, 4.0 equiv.) in 1 mL of DMF. The reaction mixture was stirred for 2 hours. A solution of EDC (72 mg, 1.3 equiv.) and pentafluorophenol (67 mg, 1.3 equiv.) in 1 mL of DMF was then added; stirring was continued for 18 hours. The reaction mixture was then diluted with 50 mL of DCM. the organic phase was washed with 1 N aqueous NaHSO₄ then brine, dried over Na₂SO₄ and concentrated under reduced pressure to afford a residue, which was purified by flash chromatography (0 to 80% ethyl acetate in hexanes) to afford 97 mg of ester I-109 (69% yield). ESI-MS found 489.9, C₂₀H₁₅F₅N₃O₄S (M−H)⁻ requires 490.1.

### Synthesis of I-111 is Depicted in Scheme 43

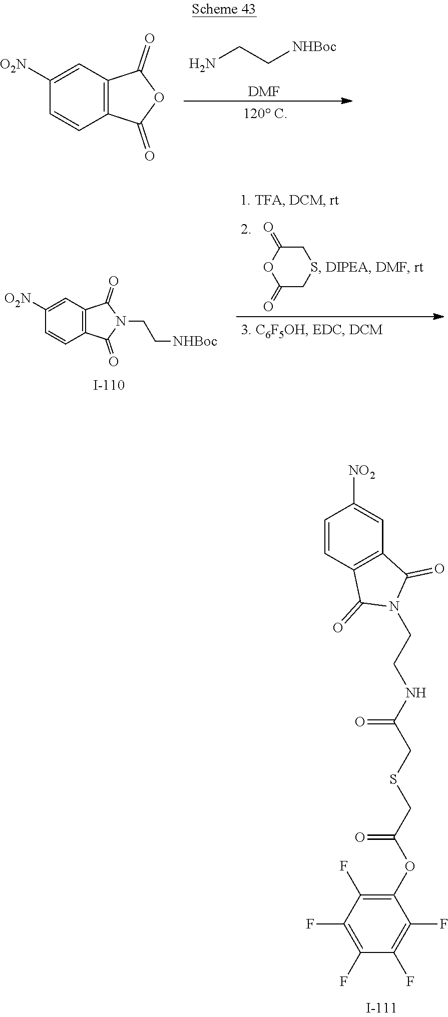

A solution of 5-nitroisobenzofuran-1,3-dione (295 mg, 1.53 mmol) and tert-butyl (2-aminoethyl)carbamate (367 mg, 1.5 equiv.) in 9 mL of anhydrous DMF was stirred for 30 minutes at ambient temperature and then for 2 hours at 120° C. The reaction mixture was allowed to cool to ambient temperature and diluted with 40 mL of EtOAc. The organic phase was washed with water and brine. The combined organic phase was then dried over sodium sulfate and concentrated under reduced pressure to a residue, which was purified by silica gel chromatography to afford 302 mg (90% yield) of carbamate I-110.

Carbamate I-110 (132 mg, 0.395 mmol) was deprotected with TFA/DCM (1:1 v/v) for two hours at ambient temperature, and the reaction mixture was concentrated under reduced pressure to afford the crude trifluoroacetate salt. The crude triflouroacetate salt was dissolved in 4 mL of DMF and treated with a solution of thiodiglycolic anhydride (58.5 mg, 1.1 equiv. based on carbamate I-110) and DIPEA (85 μL, 1.2 equiv.) in 1 mL of DCM. The reaction mixture was stirred for 18 hours and then a solution of EDC (68 mg, 0.8 equiv.) and pentafluorophenol (58 mg, 0.8 equiv.) in 1 mL of DMF was added and stirring was continued for 1 hour. The reaction mixture was then diluted with 30 mL of DCM. The organic phase was washed with 1 N aqueous NaHSO₄ and brine, dried over Na₂SO₄, and concentrated under reduced pressure to afford a residue which was purified by flash chromatography (0 to 60% ethyl acetate in hexanes) to afford 111 mg of ester I-111 (46% yield). ESI-MS found 533.8, C₂₀H₁₂F₃N₃O₇S (MH⁺) requires 534.0.

### Synthesis of I-113 is depicted in Scheme 44

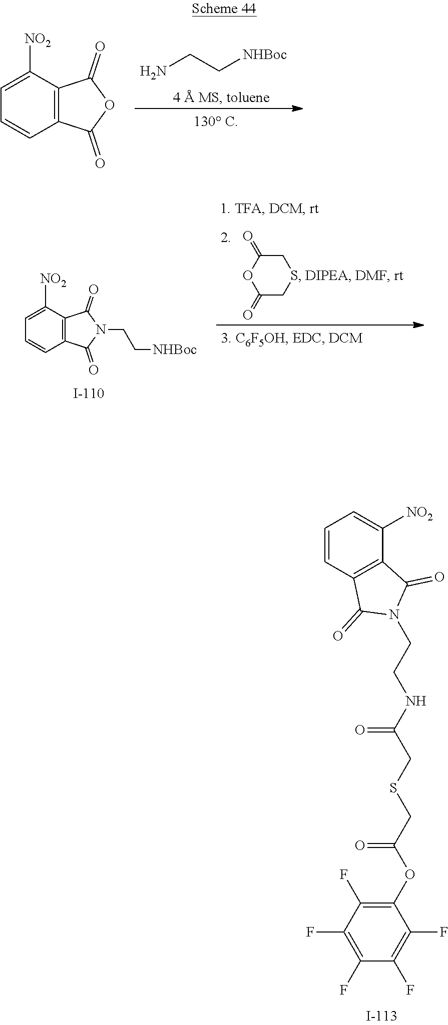

A suspension of 4-nitroisobenzofuran-1,3-dione (145 mg, 0.765 mmol) and tert-butyl (2-aminoethyl)carbamate (184 mg, 1.5 equiv.) were stirred over 4 Å molecular sieves in a sealed tube at 130° C. for 16 hours. The reaction mixture was filtered, concentrated in vacuo, and purified by silica gel chromatography to afford 82 mg (32% yield) of carbamate I-112 which was deprotected in 1:1 DCM/TFA for two hours at ambient temperature followed by concentration in vacuo to give a crude trifluoroacetate salt. The salt was dissolved in 4 mL of DMF and treated with a solution of thiodiglycolic anhydride (32 mg 0.24 mmol) and DIPEA (48 μL, 0.27 mmol) in 1 mL of DMF. The reaction mixture was stirred for 2 hours and then a solution of EDC (68 mg, 0.32 mmol) and pentafluorophenol (58 mg, 0.32 mmol) in 1 mL of DMF was added; stirring was continued for one hour. The reaction mixture was then diluted with 30 mL DCM, and the organic phase was washed with 1 N NaHSO₄ and brine, dried over Na₂SO₄, and concentrated under reduced pressure to afford a residue which was purified by flash chromatography (0 to 60% ethyl acetate in hexanes) to afford 50 mg of I-113 (39% yield). ESI-MS found 533.8, C₂₀H₁₂F₅N₃O₇S (MH⁺) requires 534.0.

### Synthesis of I-115 is Depicted in Scheme 45

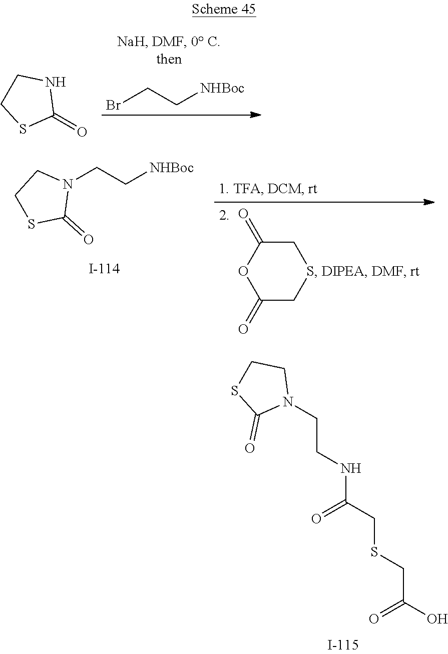

To a solution of thiazolidin-2-one (103 mg, 1.0 mmol) in 2 mL of anhydrous DMF at 0° C. was added sodium hydride (48 mg of a 60% dispersion in mineral oil, 1.2 equiv.) and the resulting suspension was stirred for 10 minutes. Tert-butyl (2-bromoethyl)carbamate (356 mg, 1.6 equiv.) in 2 mL of anhydrous DMF was added, and the solution was allowed to warm to ambient temperature and stirred for two hours. The reaction was quenched with 45 mL of saturated ammonium chloride solution. The reaction mixture was extracted three times with EtOAc. The combined organic extracts were washed with brine, dried over sodium sulfate, and concentrated in vacuo to afford a residue which was purified by silica gel chromatography to afford 125 mg (51% yield) of carbamate I-114. I-114 was deprotected in TFA/DCM (1:1 v/v) for two hours at ambient temperature followed by concentration in vacuo to give a crude trifluoroacetate salt, which was converted to 107 mg of acid I-115 (76% yield) following GP1. ESI-MS found 279.1 C₉H₁₄N₂O₄S₂ (MH⁺) requires 279.0.

### Synthesis of I-118 is Depicted in Scheme 46

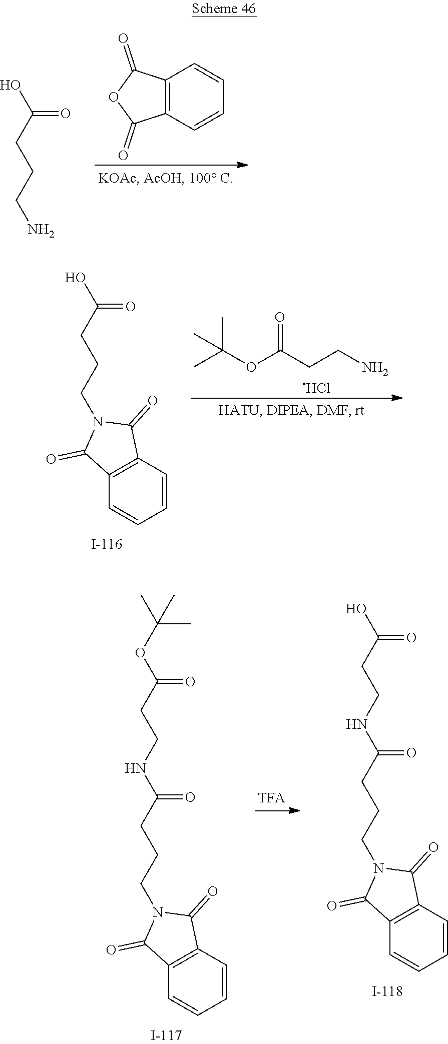

To 4-aminobutanoic acid (100 mg, 0.97 mmol) in AcOH was added phthalic anhydride (151 mg, 1.05 equiv.) and potassium acetate (286 mg, 3.0 equiv.). The reaction mixture was heated to 100° C. for 16 hours when LC-MS analysis indicated complete formation of phthalimide I-116. The reaction mixture was cooled and concentrated. The resulting residue was taken up in EtOAc; the organic extract was washed with 1M HCl, dried, and concentrated to afford the crude phthalimide I-116 which was re-dissolved in DMF. HATU (400 mg, 1.1 equiv.) was added, followed by DIPEA (607 μL, 3.5 equiv.) and B-alanine-tert-butyl ester hydrochloride (200 mg, 1.1 equiv.) The reaction mixture was stirred at ambient temperature until the reaction was deemed complete by LC-MS. The reaction mixture was concentrated to a residue, which was taken up in EtOAc and the organic extract was washed with 1M HCl, dried and concentrated. The resulting residue was purified by silica gel chromatography to afford 167 mg (48% yield, 2 steps) of phthalimide I-117. The tert-butyl ester of Phthalimide I-117 was deprotected to afford I-118 using TFA prior to coupling to the resin-bound peptide, MS (ESI) m/z calcd for C₁₅H₁₅N₂O₅ [M−H]⁻: 303.1; found 302.9.

### Synthesis of I-120 is Depicted in Scheme 47

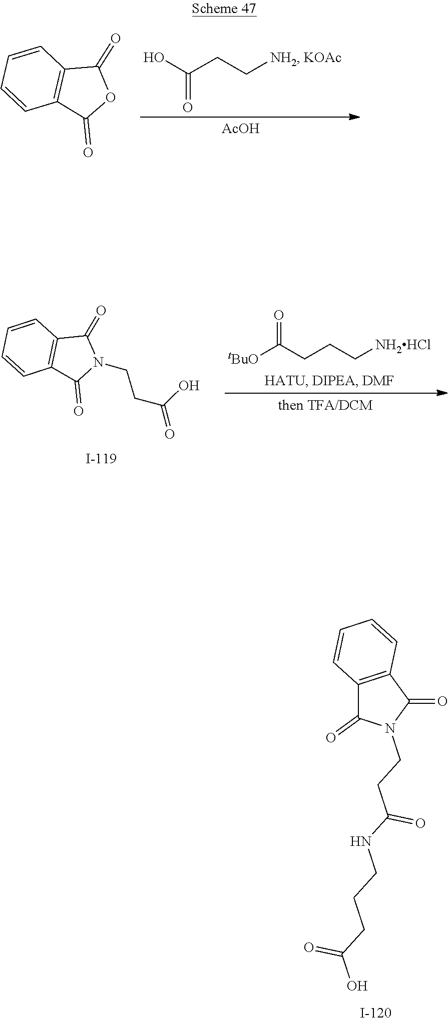

A solution of phthalic anhydride (2.26 g, 20.0 mmol), 3-aminopropanoic acid (1.87 g, 21.0 mmol) and potassium acetate (3.32 g, 1.9 equiv.) in AcOH was stirred 4 hours at 100° C., cooled to ambient temperature, and concentrated in vacuo. The resulting residue was dissolved in EtOAc, washed with 1 N HCl then brine, dried over sodium sulfate, and concentrated in vacuo to afford 4.25 g of the crude acid I-119 (97% yield). A portion of this material (59 mg, ca. 0.270 mmol) was dissolved in DMF and HATU (113 mg, 1.1 equiv.), tert-butyl 4-aminobutanoate hydrochloride (58 mg, 1.1 equiv.), and DIPEA (156 μL, 3.5 equiv.) were added. The reaction mixture was stirred at ambient temperature for 18 hours and then concentrated in vacuo. The residue was dissolved in EtOAc, and the resulting solution was washed with 1 M HCl, dried over sodium sulfate, and concentrated in vacuo. Purification by flash chromatography using hexanes/EtOAc followed by treatment with 1:1 DCM/TFA for 2 hours, concentration in vacuo and flash chromatography of the resulting residue with 20% MeOH and 1% AcOH in DCM afforded 39 mg of I-120 (108 mmol, 40% over two steps).

### Synthesis of I-122 is Depicted in Scheme 48

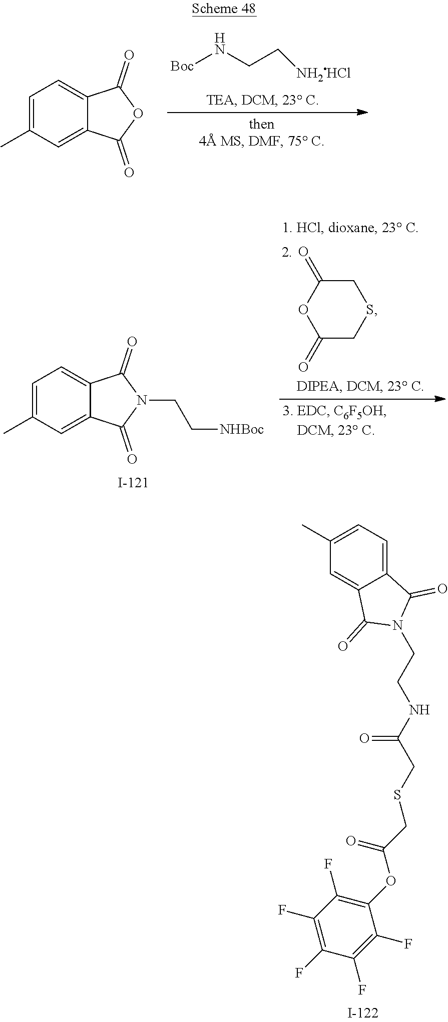

A solution of 5-methylisobenzofuran-1,3-dione (162 mg, 1.0 mmol), tert-butyl (2-aminoethyl)carbamate hydrochloride (207 mg, 1.05 equiv.) and TEA (278 μL, 2.0 equiv.) in DCM was stirred for 18 hours at ambient temperature. Then 4 Å molecular sieves and DMF were added and the reaction mixture was stirred at 75° C. for 18 hours. The reaction mixture was diluted with EtOAc, filtered, washed with saturated sodium bicarbonate and brine, then dried over sodium sulfate, and concentrated in vacuo to afford 300 mg of the intermediate imide I-121 (99% yield). This material was deprotected by treatment with 4 N HCl in dioxane followed by concentration in vacuo. A portion of the crude amine hydrochloride (30 mg, 0.125 mmol) was suspended in DCM and treated with thiodiglycolic anhydride (20 mg, 1.2 equiv.) and DIPEA (48 μL, 2.2 equiv.). The reaction mixture was stirred for 4 hours, concentrated in vacuo, and the residue was dissolved in EtOAc. The solution was washed with 1 M HCl and brine, dried over sodium sulfate, and concentrated in vacuo to afford 43 mg of the crude acid. A solution of this material, pentafluorophenol (35 mg, 0.188 mmol) and EDC (36 mg, 0.188 mmol) in DCM was stirred for one hour and then concentrated in vacuo. The residue obtained was dissolved in EtOAc, and the solution was washed with 1 M HCl, brine, dried over sodium sulfate and concentrated in vacuo. Purification by flash chromatography using EtOAc in hexanes afforded 66 mg of I-122 (quantitative yield).

### Synthesis of I-124 is Depicted in Scheme 19

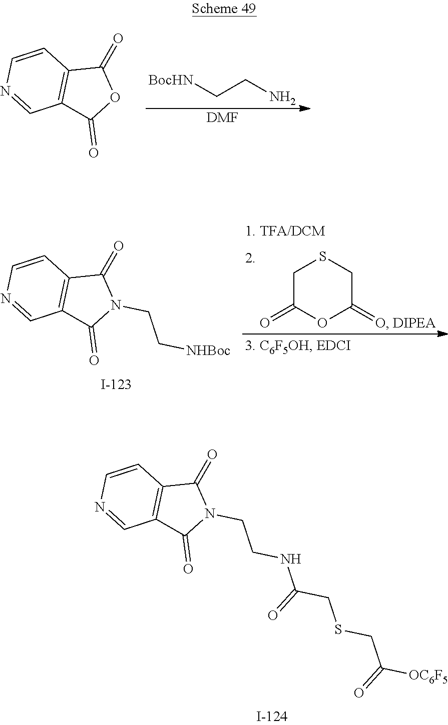

A solution of furo [3,4-c]pyridine-1,3-dione (100 mg, 0.671 mmol) and tert-butyl (2-aminoethyl)carbamate (112 mg, 1.05 equiv.) in DMF was stirred at 130° C. for 18 hours and then allowed to cool to ambient temperature. The reaction mixture was diluted with EtOAc and washed with 1 M HCl and brine. The combined aqueous washes were back-extracted with EtOAc. The combined organic extracts were dried over sodium sulfate and concentrated in vacuo. The resulting crude carbamate I-123 was stirred in 1:1 DCM/TFA for 2 hours, after which removal of the solvent afforded a primary amine salt, which was suspended in DCM and treated with thiodiglycolic anhydride (98 mg, 1.1 equiv.) and DIPEA (388 μL, 1.1 equiv.). The reaction mixture was stirred 4 hours. Pentafluorophenol (184 mg, 1.5 equiv.) and EDC (194 mg, 1.5 equiv.) were added; stirring was continued for 2 hours. The reaction mixture was diluted with EtOAc and the solution was washed with 1 M HCl then brine, dried over sodium sulfate and concentrated in vacuo. Purification by flash chromatography using EtOAc in hexanes afforded 200 mg of I-124 (61% yield).

### Synthesis of I-126 is Depicted in Scheme 50

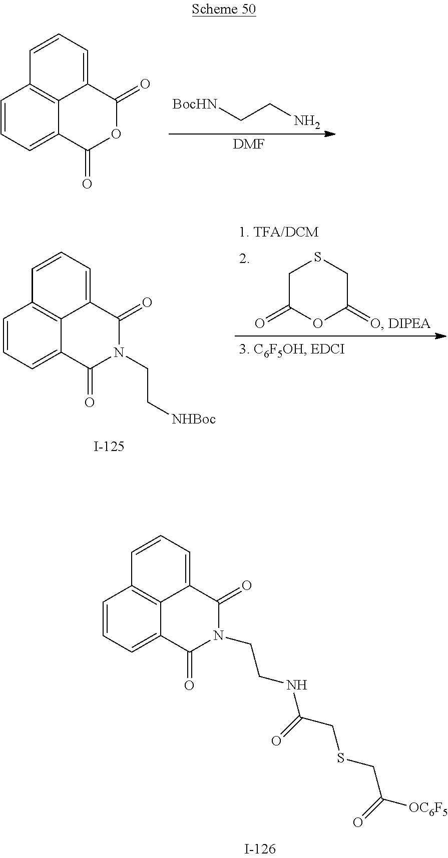

A solution of 1H,3H-benzo[de] isochromene-1,3-dione (100 mg, 0.505 mmol) and tert-butyl (2-aminoethyl)carbamate (85 mg, 1.05 equiv.) in DMF was stirred at 130° C. for 3 hours and allowed to cool to ambient temperature. The reaction mixture was diluted with EtOAc and washed with 1 M HCl and brine. The combined aqueous washes were extracted with EtOAc. The combined organic extracts were dried over sodium sulfate and concentrated in vacuo. The resulting crude carbamate I-125 was stirred in 1:1 DCM/TFA for 2 hours, after which removal of the solvent afforded a primary amine salt. The amine salt was suspended in DCM and treated with thiodiglycolic anhydride (73 mg, 1.1 equiv.) and DIPEA (219 μL, 2.5 equiv.). The reaction mixture was stirred 4 hours. Pentafluorophenol (139 mg, 1.5 equiv.) and EDC (207 mg, 2.0 equiv.) were added; stirring was continued for 2 hours. The reaction mixture was diluted with EtOAc and the solution was washed with 1 M HCl then brine, dried over sodium sulfate and concentrated in vacuo. Purification by flash chromatography using EtOAc in hexanes afforded I-126.

### Synthesis of I-127 is Depicted in Scheme 51

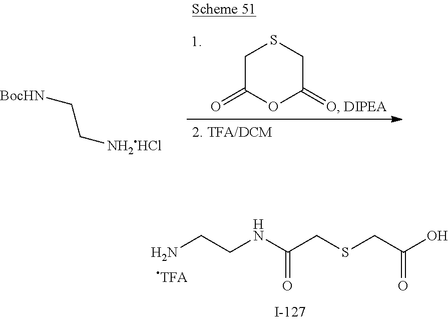

A solution of tert-butyl (2-aminoethyl)carbamate hydrochloride (207 mg, 1.05 mmol), thiodiglycolic anhydride (132 mg, 0.95 equiv.) and DIPEA (365 μL, 2.0 equiv.) in DCM was stirred 2 hours, concentrated in vacuo. The residue obtained was dissolved in EtOAc. This solution was washed with 1 M HCl and brine, dried over sodium sulfate and concentrated in vacuo to afford 366 mg of the crude carbamate, which was deprotected by treatment with 1:1 v/v TFA/DCM to afford the crude amine-TFA salt, I-127.

### Synthesis of I-129 is Depicted in Scheme 52

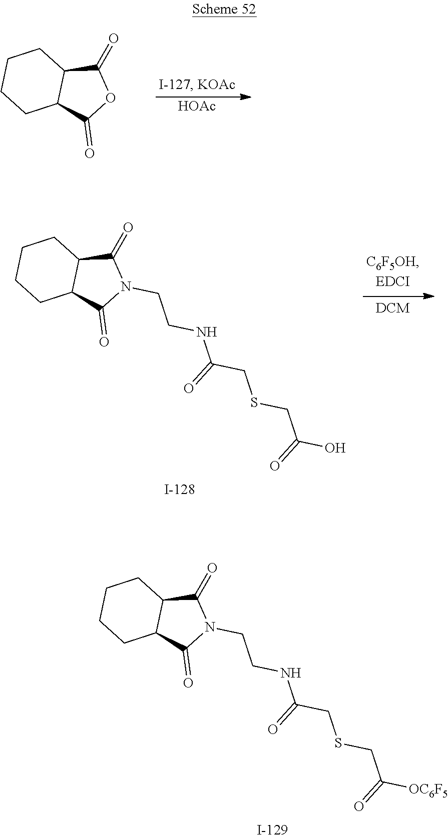

A solution of amine salt I-127 (34 mg, 0.111 mmol), (meso)-hexahydroisobenzofuran-1,3-dione (21 mg, 1.2 equiv.), and potassium acetate (33 mg 3.0 equiv.) in AcOH was heated to 100° C. for 18 hours. The resulting mixture was cooled to ambient temperature and concentrated in vacuo. The residue was dissolved in EtOAc and washed with 1 M HCl then brine, and the combined aqueous washes were back-extracted with EtOAc. The combined organic extracts were dried over sodium sulfate and concentrated in vacuo. The resulting residue was purified by flash chromatography using 15% MeOH in DCM with 1% AcOH to afford 32 mg of intermediate acid I-128 (88% yield). This material was dissolved in DCM along with pentafluorophenol (18.4 mg, 0.100 mmol) and EDC (19 mg, 0.100 mmol). The resulting reaction mixture was stirred for 2 hours and concentrated in vacuo. The residue was dissolved in EtOAc. The resulting solution was washed with 1 M HCl and brine, then dried over sodium sulfate and concentrated in vacuo. Purification of the residue by flash chromatography using EtOAc in hexanes afforded 43 mg of I-129 (78% yield over two steps).

### Synthesis of I-131 is Depicted in Scheme 53

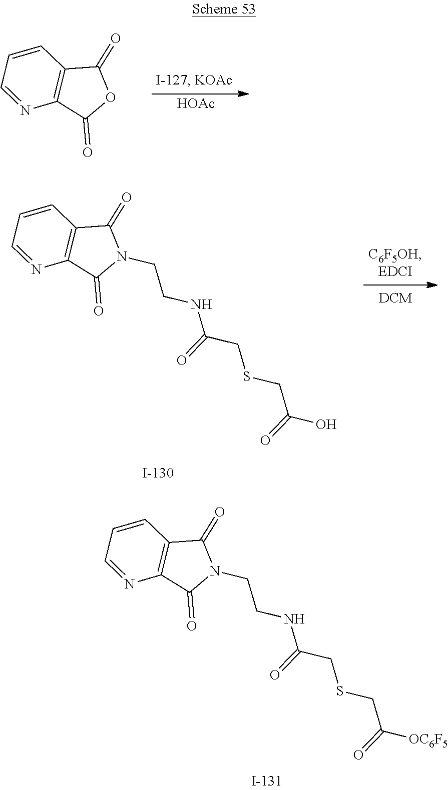

A solution of amine salt I-127 (34 mg, 0.111 mmol), furo[3,4-b]pyridine-5,7-dione (20 mg, 1.2 equiv.) and potassium acetate (33 mg, 3.0 equiv.) in AcOH was heated to 100° C. for 18 hours then cooled to ambient temperature and concentrated in vacuo. The residue was dissolved in EtOAc and the solution was washed with 1 M HCl and then brine. The combined aqueous washes were back-extracted with EtOAc. The combined organic extracts were dried over sodium sulfate and concentrated in vacuo. The resulting residue was purified by flash chromatography using 15% MeOH in DCM with 1% AcOH to afford 14 mg of intermediate acid I-130 (39% yield). This material was dissolved in DCM along with pentafluorophenol (12 mg, 0.065 mmol) and EDC (16 mg, 0.086 mmol). The resulting solution was stirred for 2 hours, concentrated in vacuo and the residue was dissolved in EtOAc. The resulting solution was washed with 1 M HCl and brine, then dried over sodium sulfate and concentrated in vacuo. Purification of the residue by flash chromatography using EtOAc in hexanes afforded 17 mg of I-131 (81% yield).

### Synthesis of I-133 is Depicted in Scheme 54

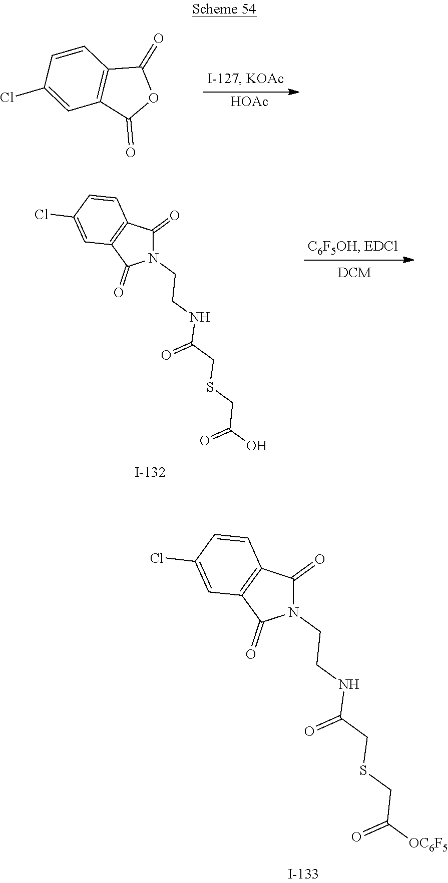

### Scheme 54

A solution of amine salt I-127 (30 mg, 0.098 mmol), 5-chloroisobenzofuran-1,3-dione (21 mg, 1.2 equiv.), and potassium acetate (29 mg, 3.0 equiv.) in AcOH was heated to 100° C. for 18 hours then cooled to ambient temperature and concentrated in vacuo. The residue was dissolved in EtOAc and the solution was washed with 1 M HCl and then brine. The combined aqueous washes were extracted with EtOAc. The combined organic extracts were dried over sodium sulfate and concentrated in vacuo. The resulting residue was purified by flash chromatography using 15% MeOH in DCM with 1% AcOH to afford 29 mg of intermediate acid I-132 (69%). This material was dissolved in DCM along with pentafluorophenol (18 mg, 0.100 mmol) and EDC (26 mg, 0.139 mmol). The resulting reaction mixture was stirred for 2 hours and concentrated in vacuo; the residue was re-dissolved in EtOAc. The resulting solution was washed with 1 M HCl and brine, dried over sodium sulfate, concentrated in vacuo. Purification of the residue by flash chromatography using EtOAc in hexanes afforded 21 mg of I-133 (60% yield).

### Synthesis of I-135 is Depicted in Scheme 55

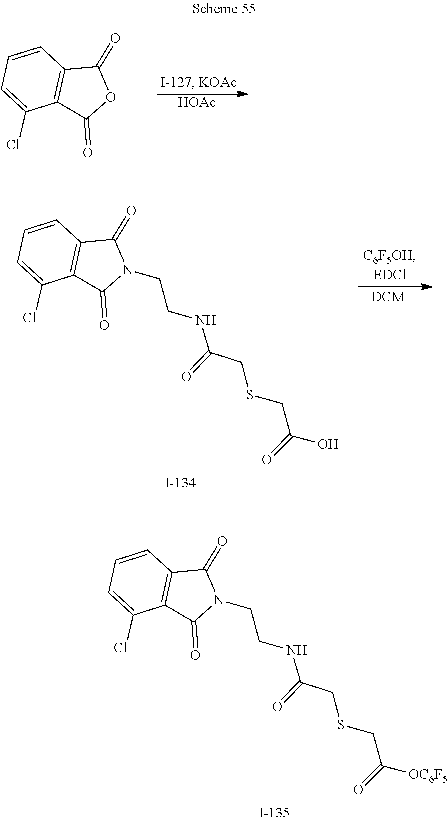

A solution of amine salt I-127 (23 mg, 0.075 mmol), 4-chloroisobenzofuran-1,3-dione (16 mg, 1.2 equiv.) and potassium acetate (22 mg, 3.0 equiv.) in AcOH was heated to 100° C. for 18 hours, cooled to ambient temperature, and concentrated in vacuo. The residue was dissolved in EtOAc and washed with 1 M HCl then brine, and the combined aqueous washes were back-extracted with EtOAc. The combined organic extracts were dried over sodium sulfate and concentrated in vacuo. The resulting residue was purified by flash chromatography using 15% MeOH in DCM with 1% AcOH to afford 21 mg of intermediate acid I-134 (79% yield). This material was dissolved in DCM along with pentafluorophenol (16 mg, 0.089 mmol) and EDC (23 mg, 0.118 mmol). The resulting reaction mixture was stirred for 2 hours and concentrated in vacuo. The residue was dissolved in EtOAc. The resulting solution was washed with 1 M HCl and brine, then dried over sodium sulfate and concentrated in vacuo. Purification of the residue by flash chromatography using EtOAc in hexanes afforded 20 mg of I-135 (0.038 mmol, 65% yield).

### Synthesis of I-137 is Depicted in Scheme 56

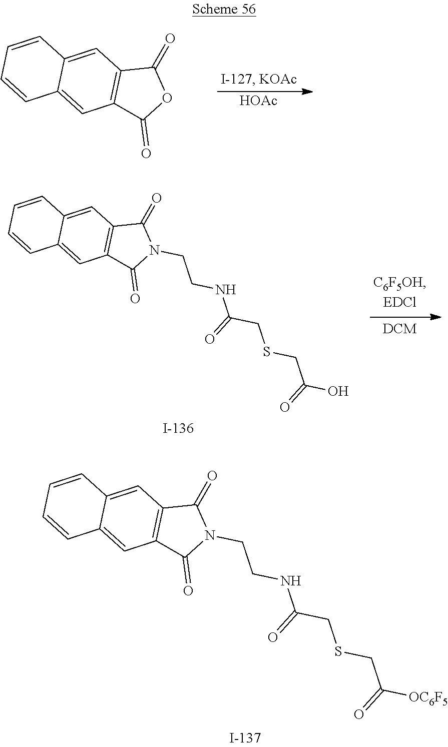

A solution of amine salt I-127 (43 mg, 0.14 mmol), naphtho[2,3-c]furan-1,3-dione (33 mg, 1.2 equiv.), and potassium acetate (42 mg, 3.0 equiv.) in AcOH was heated to 100° C. for 18 hours. The reaction mixture was then cooled to ambient temperature and concentrated in vacuo. The residue afforded was dissolved in EtOAc, and the solution was washed with 1 M HCl and brine. The combined aqueous washes were back-extracted with EtOAc. The combined organic extracts were dried over sodium sulfate and concentrated in vacuo. The resulting residue was purified by flash chromatography using 15% MeOH in DCM with 1% AcOH to afford 20 mg of intermediate acid I-136 (38% yield). This material was dissolved in DCM along with pentafluorophenol (15 mg, 0.081 mmol) and EDC (16 mg, 0.081 mmol). The resulting reaction mixture was stirred for 2 hours, concentrated in vacuo and the residue was re-dissolved in EtOAc. The resulting solution was washed with 1 M HCl and brine, then dried over sodium sulfate and concentrated in vacuo. Purification of the residue by flash chromatography using EtOAc in hexanes afforded 31 mg of I-137 (98% yield).

### Synthesis of I-139 is Depicted in Scheme 57

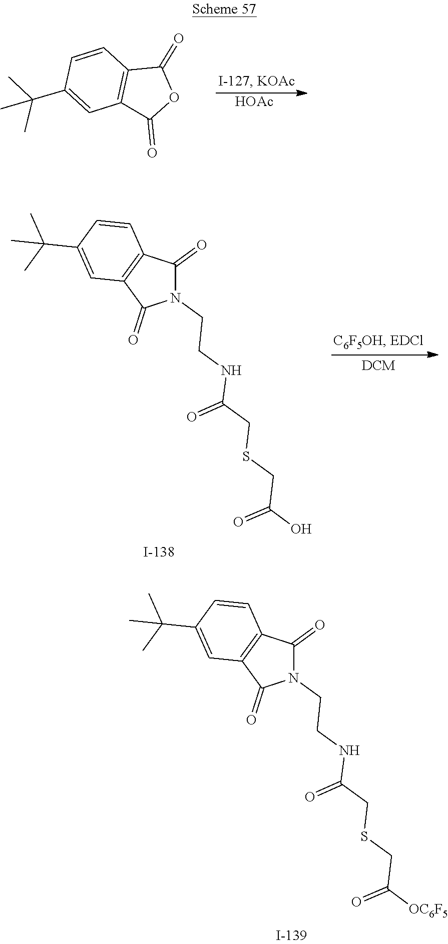

A solution of amine salt I-127 (36 mg, 0.117 mmol), 5-(tert-butyl) isobenzofuran-1,3-dione (29 mg, 1.2 equiv.) and potassium acetate (11 mg, 1.0 equiv.) in AcOH was heated to 100° C. for 18 hours. The reaction mixture was then cooled to ambient temperature and concentrated in vacuo. The residue was dissolved in EtOAc and the solution was washed with 1 M HCl then brine, and the combined aqueous washes were back-extracted with EtOAc. The combined organic extracts were dried over sodium sulfate and concentrated in vacuo. The resulting residue was purified by flash chromatography using 15% MeOH in DCM with 1% AcOH to afford 49 mg of intermediate acid I-138 containing dicarboxylic acid impurities. This material was dissolved in DCM along with pentafluorophenol (18.4 mg, 0.100 mmol) and EDC (19 mg, 0.100 mmol). The resulting reaction mixture was stirred for 2 hours, concentrated in vacuo, and the residue was re-dissolved in EtOAc. The resulting solution was washed with 1 M HCl and brine, dried over sodium sulfate, and concentrated in vacuo. Purification of the residue by flash chromatography using EtOAc in hexanes afforded 22 mg of I-139 (37% yield over two steps).

### Synthesis of I-141 is Depicted in Scheme 58

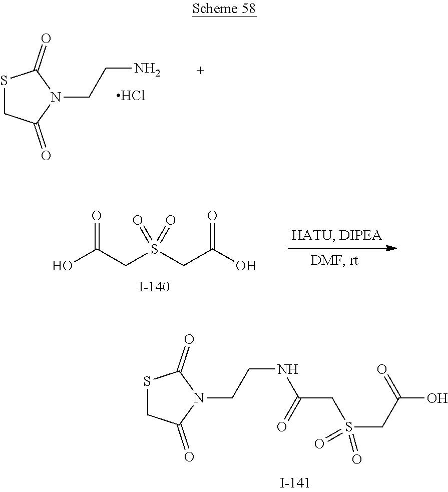 [SMILES: NCCN1C(=O)CSC1=O ; Cl ; O=C(O)CS(=O)(=O)CC(=O)O ; O=C(O)CS(=O)(=O)CC(=O)NCCN1C(=O)CSC1=O]

To a slurry of 3-(2-aminoethyl) thiazolidine-2,4-dionehydrochloride (43.8 mg, 0.22 mmol) and dicarboxylic acid I-140 (81.1 mg, 2.0 equiv.) in DMF (1 mL) was added DIPEA (117 μL, 3.0 equiv.) and HATU (127.2 mg, 1.5 equiv.). The reaction mixture was agitated at ambient temperature for 2.5 hours and then purified directly by preparative HPLC (0-100% acetonitrile in water with 0.1% TFA) to afford 62.1 mg (86% yield) of I-141 as a white solid. ESI-MS found 325.1, C₉H₁₃N₂O₇S₂ (MH⁺) requires 325.1.

### Synthesis of I-145 is Depicted in Scheme 59

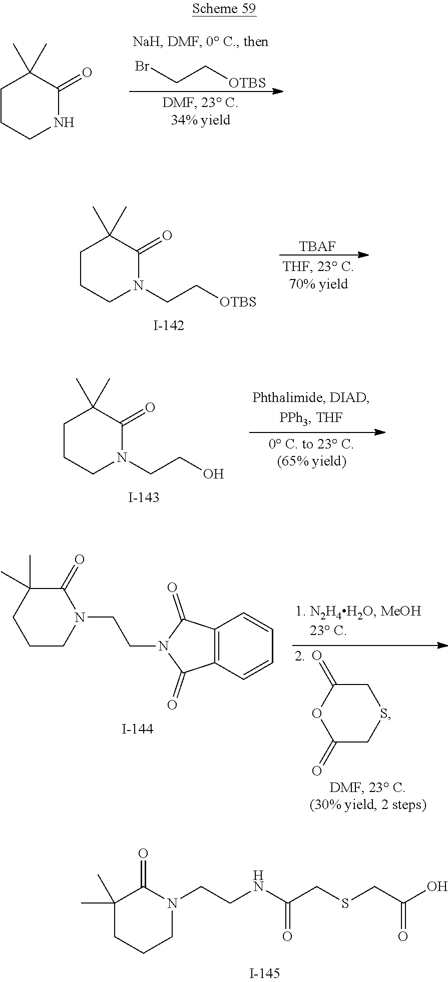

To a solution of 3,3-dimethylpiperidin-2-one (240 mg, 1.88 mmol) in DMF (5 mL) at 0° C. under N₂ was added a slurry of NaH (574 mg of 60% dispersion in mineral oil, 3.0 equiv.) in DMF (15 mL). After 20 minutes, (2-bromoethoxy)(tert-butyl)dimethylsilane (673 mg, 1.5 equiv.) was added. The reaction mixture was allowed to warm to ambient temperature. After 18 hours, the reaction mixture was quenched with saturated aqueous NH₄Cl (10 mL) and diluted with water (50 mL). The aqueous phase was extracted with EtOAc (3×50 mL). The combined EtOAc extracts were dried (Na₂SO₄) and concentrated to afford a colorless oil. Purification by silica gel chromatography (12 g pre-packed column, 0 to 25% EtOAc in Hexanes) afforded 181 mg (34% yield) of the silyl ether I-142 as a colorless oil. This material (90 mg, 0.315 mmol) was dissolved in THF (3 mL) and treated with 1M TBAF solution in THF (0.95 mL, 3 equiv.) and the resulting solution was stirred at ambient temperature for 2 hours. The reaction mixture was then concentrated under reduced pressure to afford a residue, which was purified by preparative HPLC (H₂O/MeCN with 0.1% TFA) to provide 37.7 mg of alcohol I-143 (70% yield).

Alcohol I-143 was dissolved in THF (6 mL) in the presence of triphenylphosphine (85.7 mg, 1.5 equiv.) and phthalimide (48.1 mg, 1.5 equiv.). The solution was cooled to 0-5° C., and DIAD (64 μL, 1.5 equiv.) was added; the reaction mixture was allowed to warm to ambient temperature. After 1.5 hours, the reaction mixture was concentrated to a yellow oil, which was purified by silica gel chromatography (12 g pre-packed column, eluting with 0 to 35% Acetonitrile in DCM) to afford 42.5 mg of phthalimide I-144 (65% yield) as a colorless oil.

To a solution of phthalimide I-144 (42.5 mg, 0.142 mmol) in MeOH (2 mL) was added hydrazine hydrate (44.2 μL, 10.0 equiv.). The reaction mixture was heated to 45° C. for 2 hours, cooled and concentrated under reduced pressure. The residue was re-dissolved in DMF (2 mL) and thiodiglycolic anhydride (22.5 mg, 1.2 equiv. based on phthalimide I-144) was added. After 2 hours, the reaction mixture was subjected to purification by preparative HPLC (H₂O/MeCN with 0.1% TFA) to afford 13.0 mg (30% yield) of the desired carboxylic acid I-145 as a colorless oil. ESI-MS: Found 301.2, C₁₃H₂₁N₂O₄S (M−H)⁻ requires 301.1.

### Synthesis of I-146 is Depicted in Scheme 60

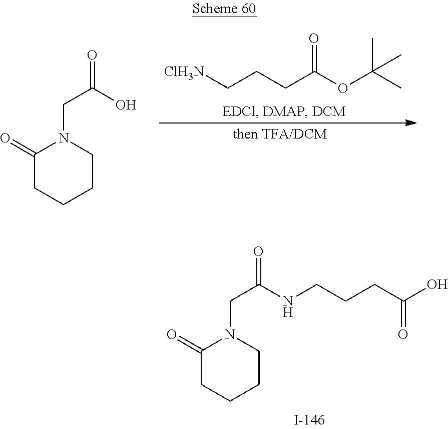

A suspension of 2-(2-oxopiperidin-1-yl)acetic acid (46 mg, 0.292 mmol) and tert-butyl 4-aminobutanoate hydrochloride (68 mg, 1.2 equiv.) in 0.5 mL of DCM was treated with DMAP (78 mg, 2.2 equiv.) and then EDC (67 mg, 1.2 equiv.). The mixture was allowed to stir at ambient temperature for 72 hours. The reaction mixture was diluted with 8 mL of DCM, and the organic phase was washed with 1 N NaHSO₃, brine, dried over Na₂SO₄, and concentrated in vacuo. The resulting crude ester was dissolved in 1 mL of DCM and treated with 1 mL of TFA, and the resulting solution was concentrated under a stream of nitrogen and held under high vacuum for 2 hours. Half of the crude product was purified by preparative HPLC (H₂O/MeCN with 0.1% TFA) to afford 27.6 mg (78% yield based on 0.146 mmol) of carboxylic acid I-146. ESI-MS: Found 243.3, C₁₁H₁₉N₂O₄ (MH⁺) requires 243.3.

### Synthesis of I-147 is Depicted in Scheme 61

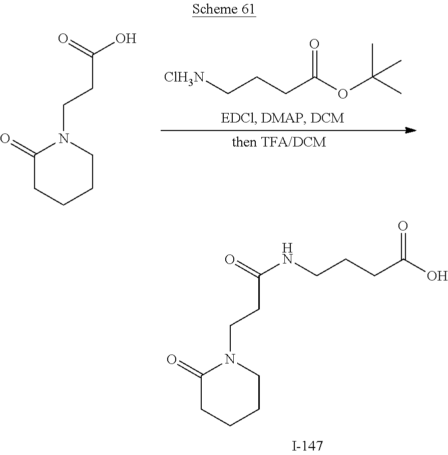

A suspension of 3-(2-oxopiperidin-1-yl) propanoic acid (50 mg, 0.292 mmol) and tert-butyl 4-aminobutanoate hydrochloride (68 mg, 1.2 equiv.) in 0.5 mL of DCM was treated with DMAP (78 mg, 2.2 equiv.) and then EDC (67 mg, 1.2 equiv.). The mixture was allowed to stir at ambient temperature for 72 hours. The reaction mixture was diluted with 8 mL of DCM, and the organic phase was washed with 1 N NaHSO₃, brine, dried over Na₂SO₄ and concentrated in vacuo. The resulting crude ester was dissolved in 1 mL of DCM and treated with 1 mL of TFA and the resulting solution was concentrated under a stream of nitrogen and held under high vacuum for 2 hours. Half of the crude product was then purified by preparative HPLC (H₂O/MeCN with 0.1% TFA) to afford 32.8 mg (87% yield based on 0.146 mmol) of carboxylic acid I-147. ESI-MS: Found 255.1, C₁₂H₁₉N₂O₄ (M−H)⁻ requires 255.3.

### Synthesis of I-148 is Depicted in Scheme 62

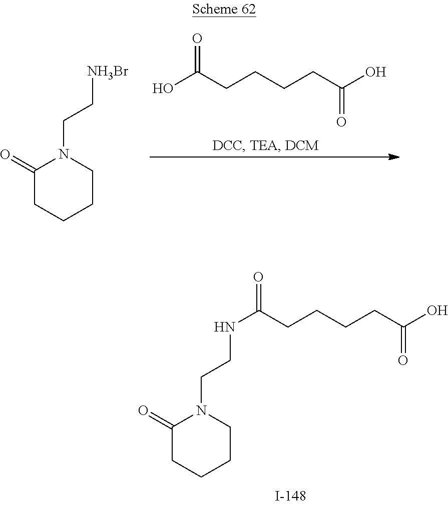

To a suspension of 1-(2-aminoethyl)piperidin-2-one hydrobromide (71.2 mg, 0.319 mmol), adipic acid (54.0 mg, 1.2 equiv.) and DCC (78.8 mg, 1.2 equiv.) was added TEA (53 μL, 1.2 equiv.). The resulting solution was stirred at ambient temperature for 72 hours, then filtered through celite, and concentrated in vacuo. Purification by preparative HPLC (H₂O/MeCN with 0.1% TFA) afforded 24 mg (23% yield) of carboxylic acid I-148. ESI-MS: Found 269.3, C₁₃H₂₁N₂O₄ (M−H)⁻ requires 269.3.

### Synthesis of I-150 is Depicted in Scheme 63

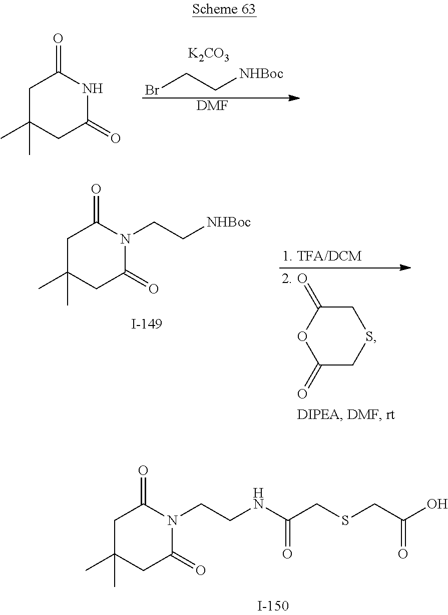

To a solution of 4,4-dimethylpiperidine-2,6-dione (28 mg, 0.20 mmol) and tert-butyl (2-bromoethyl)carbamate (45 mg, 1.0 equiv.) in 1 mL of anhydrous DMF was added potassium carbonate (41 mg, 1.5 equiv.), and the resulting suspension was stirred for 22 hours at ambient temperature. The reaction mixture was filtered and purified directly by preparative HPLC to afford 23 mg (40% yield) of carbamate I-149. Carbamate I-149 was subjected to deprotection in 2:1 DCM/TFA for 10 minutes at ambient temperature. The reaction mixture was then concentrated in vacuo to give a crude trifluoroacetate salt, which was converted to 19 mg (74% yield) of acid I-150 using GP1. ESI-MS found 317.0, C₁₃H₂₀N₂O₅S (MH⁺) requires 317.1.

### Synthesis of I-152 is Depicted in Scheme 64

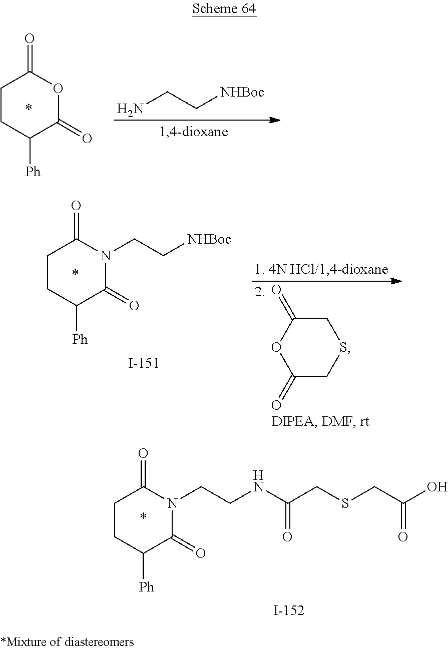 [SMILES: O=C1CCC(c2ccccc2)C(=O)O1 ; CC(C)(C)OC(=O)NCCN ; CC(C)(C)OC(=O)NCCN1C(=O)CCC(c2ccccc2)C1=O ; O=C1CSCC(=O)O1 ; O=C(O)CSCC(=O)NCCN1C(=O)CCC(c2ccccc2)C1=O]

A solution of rac-3-phenyldihydro-2H-pyran-2,6(3H)-dione (38 mg, 0.20 mmol) and tert-butyl (2-aminoethyl)carbamate (38 mg, 1.2 equiv.) in 0.6 mL of anhydrous 1,4-dioxane was stirred for 35 minutes at ambient temperature. HCl in 1,4-dioxane (4N, 1.5 mL) was then added and the reaction mixture was stirred for an additional 8 days. The reaction mixture was then concentrated in vacuo to give a crude hydrochloride salt, which was converted to 17.5 mg of I-152 (39% yield overall) following GP1. ESI-MS found 365.0, C₁₇H₂₀N₂O₅S (MH⁺) requires 365.1.

### Synthesis of I-155 is Depicted in Scheme 65

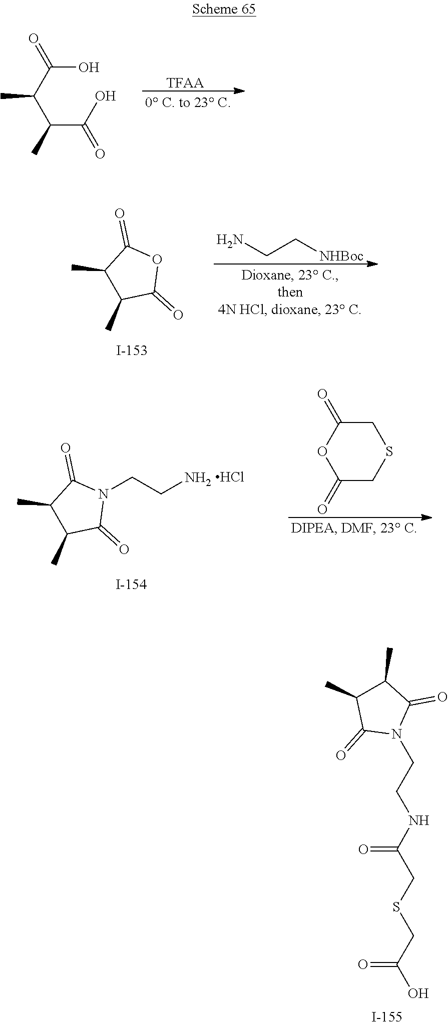

Meso-2,3-dimethylsuccinic acid (44 mg, 0.30 mmol) was dissolved in 0.2 mL of TFAA at 0° C. The solution was allowed to slowly warm to room temperature and stirred for 70 minutes. Concentration of the reaction mixture in vacuo gave the crude anhydride I-153, which was dissolved in 1 mL of anhydrous 1,4-dioxane and treated with tert-butyl (2-aminoethyl)carbamate (58 mg, 1.2 equiv.). The reaction mixture was stirred for 40 minutes at ambient temperature. 4N HCl in 1,4-dioxane (1.5 mL) was then added, and the reaction mixture was stirred for an additional 136 hours and then concentrated in vacuo to give a crude hydrochloride salt I-154 which was converted to 52 mg of carboxylic acid I-155 (57% yield overall) following GP1. ESI-MS found 303.0, C₁₂H₁₈N₂O₅S (MH⁺) requires 303.1.

### Synthesis of I-157 is Depicted in Scheme 66

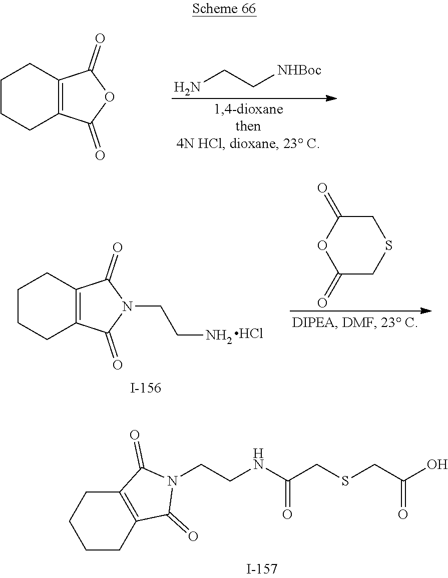 [SMILES: O=C1OC(=O)C2=C1CCCC2 ; CC(C)(C)OC(=O)NCCN ; NCCN1C(=O)C2=C(CCCC2)C1=O ; Cl ; O=C1CSCC(=O)O1 ; O=C(O)CSCC(=O)NCCN1C(=O)C2=C(CCCC2)C1=O]

A solution of 4,5,6,7-tetrahydroisobenzofuran-1,3-dione (46 mg, 0.30 mmol) and tert-butyl (2-aminoethyl)carbamate (58 mg, 1.2 equiv.) in 0.8 mL of anhydrous 1,4-dioxane was stirred 50 minutes at ambient temperature. 4N HCl in 1,4-dioxane (1 mL) was then added, and the reaction mixture was stirred for an additional 5 hours and then concentrated in vacuo to give a crude hydrochloride salt I-156, which was converted to 55.5 mg of I-157 (57% yield overall) following GP1. ESI-MS found 327.0, C₁₄H₁₈N₂O₅S (MH⁺) requires 327.1.

### Synthesis of I-159 is Depicted in Scheme 67

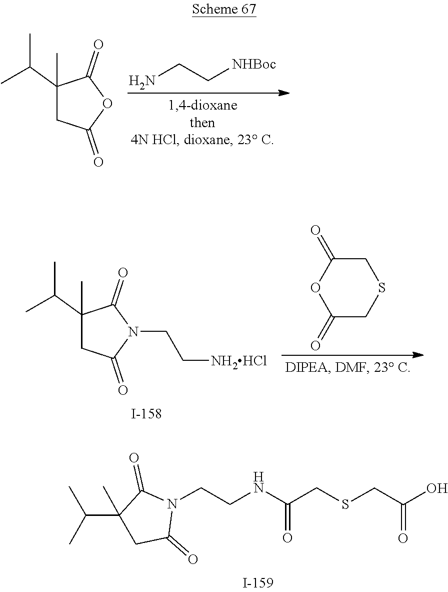

A solution of rac-3-isopropyl-3-methyldihydrofuran-2,5-dione (47 mg, 0.30 mmol) and tert-butyl (2-aminoethyl)carbamate (58 mg, 1.2 equiv.) in 0.8 mL anhydrous 1,4-dioxane was stirred for 15 minutes at ambient temperature. 4N HCl in 1,4-dioxane (0.8 mL) was then added, and the reaction mixture was stirred for an additional 41 hours and then concentrated in vacuo to give a crude hydrochloride salt I-158, which was converted to 82.0 mg of I-159 (83% yield overall) following GP1. ESI-MS found 331.0, C₁₄H₂₂N₂O₅S (MH⁺) requires 331.1.

### Synthesis of I-161 is Depicted in Scheme 68

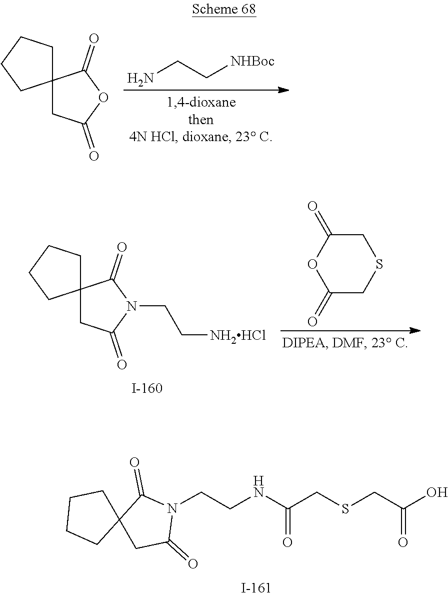

A solution of 2-oxaspiro[4.4]nonane-1,3-dione (46 mg, 0.30 mmol) and tert-butyl (2-aminoethyl)carbamate (58 mg, 1.2 equiv.) in 0.8 mL of anhydrous 1,4-dioxane was stirred for 70 minutes at ambient temperature. 4N HCl in 1,4-dioxane (1 mL) was then added, and the reaction mixture was stirred for an additional 66 hours and then concentrated in vacuo to give a crude hydrochloride salt I-160, which was converted to 66.0 mg of I-161 (67% yield overall) following GP1. ESI-MS found 329.0, C₁₄H₂₀N₂O₅S (MH⁺) requires 329.1.

### Synthesis of I-163 is Depicted in Scheme 69

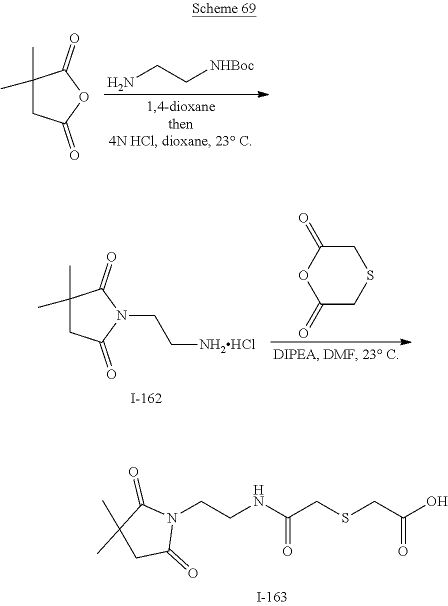

A solution of 3,3-dimethyldihydrofuran-2,5-dione (38 mg, 0.30 mmol) and tert-butyl (2-aminoethyl)carbamate (58 mg, 1.2 equiv.) in 0.8 mL of anhydrous 1,4-dioxane was stirred for 100 minutes at ambient temperature. Then 4N HCl in 1,4-dioxane (0.8 mL) was then added, and the reaction mixture was stirred for an additional 69 hours and then concentrated in vacuo to give a crude hydrochloride salt I-162, which was converted to 62.5 mg of I-163 (69% yield overall) following GP1. ESI-MS found 303.0, C₁₂H₁₈N₂O₅S (MH⁺) requires 303.1.

### Synthesis of I-165 is Depicted in Scheme 70

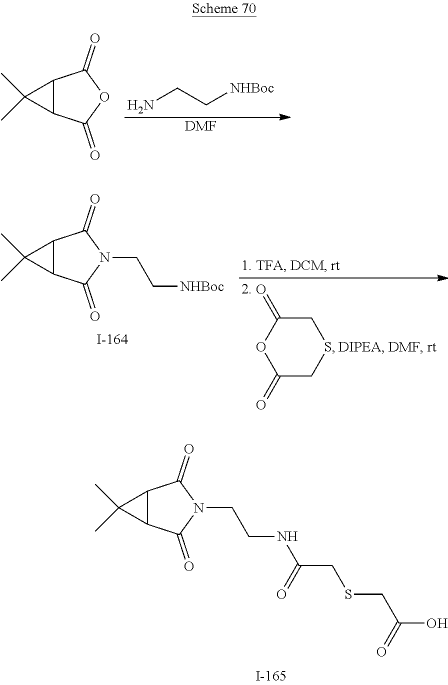

A solution of 6,6-dimethyl-3-oxabicyclo[3.1.0]hexane-2,4-dione (70 mg, 0.50 mmol) and tert-butyl (2-aminoethyl)carbamate (120 mg, 1.5 equiv.) in 3 mL of anhydrous DMF was stirred for 30 minutes at ambient temperature and then for 91 hours at 120° C. The reaction mixture was purified directly by preparative HPLC to afford 99 mg (0.35 mmol, 70% yield) of carbamate I-164. A portion of the carbamate I-164 (56 mg, 0.20 mmol) was deprotected in 2:1 v/v DCM/TFA for twenty minutes at ambient temperature. The reaction mixture was concentrated in vacuo to give a crude trifluoroacetate salt, which was converted to 56 mg of I-165 (89% yield) following GP1. ESI-MS found 315.0, C₁₃H₁₈N₂O₅S (MH⁺) requires 315.1.

### Synthesis of I-167 is Depicted in Scheme 71

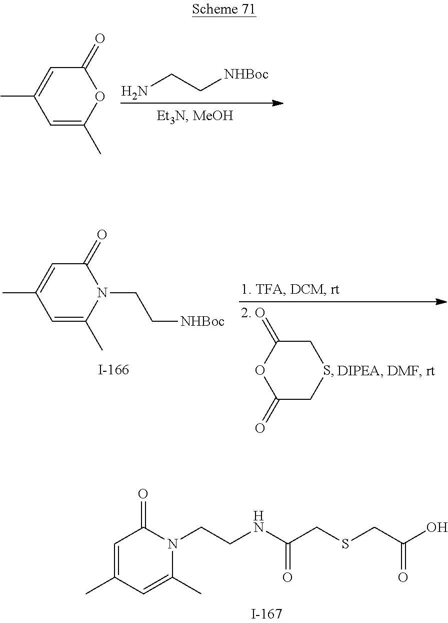

To 4,6-dimethyl-2H-pyran-2-one (222.8 mg, 1.79 mmol) in MeOH (5 mL) was added N-tert-butyloxycarbonylethylene diamine (496 mg, 1.7 equiv.) and TEA (0.75 mL, 3.0 equiv.). The reaction mixture was heated to 60° C. for 48 hours, then to 100° C. for a further 5 hours to ensure completion of the reaction. The reaction mixture was concentrated and purified by silica gel chromatography to afford the intermediate carbamate, I-166.

Carbamate I-166 (33 mg, 0.12 mmol) was treated with TFA for deprotection. After concentration of the reaction mixture, the resulting amine salt was converted to 33.4 mg of I-167 (90% yield) following GP1. ESI-MS found 299.8, C₁₃H₁₉N₂O₄S (MH⁺) requires 299.1.

### Synthesis of I-168 is Depicted in Scheme 72

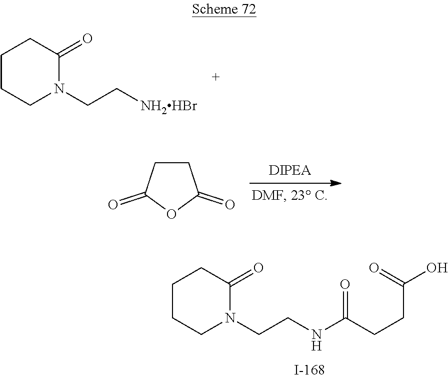

1-(2-aminoethyl)piperidin-2-one hydrobromide (54.5 mg, 0.24 mmol) was dissolved in DMF (1 mL). DIPEA (128 μL, 3.0 equiv.) was added, followed by succinic anhydride (26.9 mg, 1.1 equiv.). The reaction mixture was agitated at ambient temperature for 3 hours and then directly purified by preparative HPLC (H₂O/MeCN with 0.1% TFA) to afford 48.2 mg (81% yield) of acid I-168 as a white solid. ESI-MS found 243.3. C₁₁H₁₉N₂O₄ (MH⁺) requires 243.1.

### Synthesis of I-169 is Depicted in Scheme 73

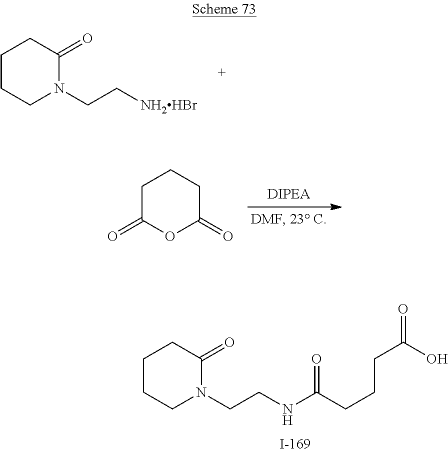

1-(2-aminoethyl)piperidin-2-one hydrobromide (64.9 mg, 0.29 mmol) was dissolved in DMF (1 mL). DIPEA (152 μL, 3.0 equiv.) was added, followed by glutaric anhydride (36.5 mg, 1.1 equiv.). The reaction mixture was agitated at ambient temperature for 3 hours and then directly purified by preparative HPLC (H₂O/MeCN with 0.1% TFA) to afford 76.8 mg (quantitative yield) of acid I-169 as a white solid. ESI-MS found 256.1. C₁₂H₂₁N₂O₄ (MH⁺) requires 256.1.

### Synthesis of I-170 is Depicted in Scheme 74

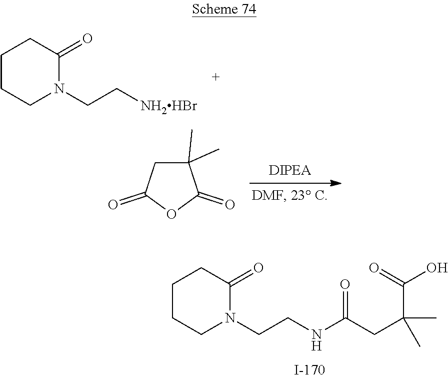

1-(2-aminoethyl)piperidin-2-one hydrobromide (178.2 mg, 0.8 mmol) was dissolved in DMF (0.9 mL). DIPEA (417 μL, 3.0 equiv.) was added, followed by 2,2-dimethylsuccinic anhydride (133 mg, 1.3 equiv.). The reaction mixture was agitated at ambient temperature for 12 hours and then directly purified by preparative HPLC (H₂O/MeCN with 0.1% TFA) to afford 51.7 mg (24% yield) of acid I-170 as a white solid. ESI-MS found 271.2. C₁₃H₂₃N₂O₄ (MH⁺) requires 271.2.

### Synthesis of I-172 is Depicted in Scheme 75

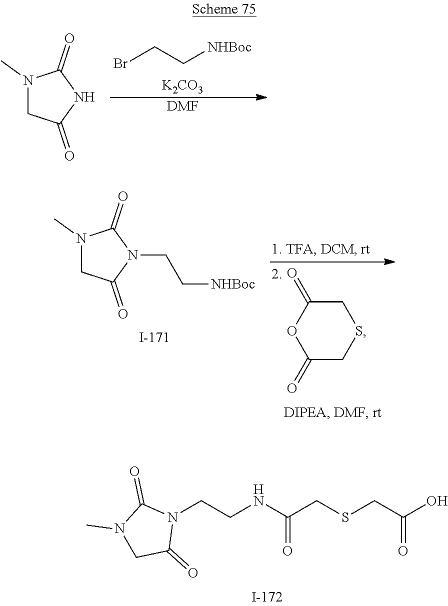

To a solution of 1-methylimidazolidine-2,4-dione (44 mg, 0.20 mmol) and tert-butyl (2-bromoethyl)carbamate (377 mg, 8.4 equiv.) in 1.5 mL of anhydrous DMF was added potassium carbonate (41 mg, 0.30 mmol) and the resulting suspension was stirred for 20 hours at ambient temperature. The reaction mixture was then filtered, and the filtrate was subjected to preparative HPLC to afford 23 mg (0.089 mmol, 45% yield) of carbamate I-171 which was deprotected in 2:1 v/v DCM/TFA for 15 minutes at ambient temperature. The reaction mixture was concentrated under reduced pressure to give a crude trifluoroacetate salt. A portion of this salt (66 mg, 0.218 mmol) was converted to 9 mg of acid I-172 using GP1 (35% yield). ESI-MS found 290.0, C₁₀H₁₅N₃O₅S (MH⁺) requires 290.1.

### Synthesis of I-174 is Depicted in Scheme 76

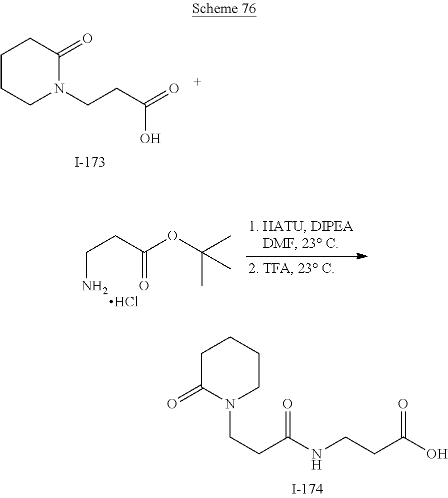

To a solution of carboxylic acid I-173 (125.3 mg, 0.73 mmol) and beta-alanine tert-butyl ester hydrochloride (146.3 mg, 1.1 equiv.) in DMF (3 mL) was added DIPEA (382 μL, 3.0 equiv.), followed by HATU (417.4 mg, 1.5 equiv.). The reaction mixture was stirred at ambient temperature for 16 hours, then diluted with H₂O (30 mL) and extracted with EtOAc (3×30 mL). The combined organic extracts were washed with brine (30 mL), dried (MgSO₄), and concentrated to afford a brown oil. TFA (5 mL) was added to this oil and the reaction mixture was stirred for 1 hour at ambient temperature. The reaction mixture was then concentrated and purified by preparative HPLC (H₂O/MeCN with 0.1% TFA) to afford I-174 (153.4 mg, 87% yield over 2 steps) as a colorless oil. ESI-MS found 243.3, C₁₁H₁₉N₂O₄ (MH⁺) requires 243.1.

### Synthesis of I-176 is Depicted in Scheme 77

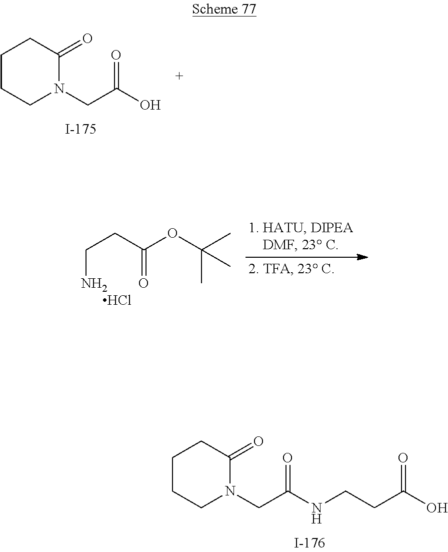

Using a procedure similar to that employed for the synthesis of I-174, carboxylic acid I-175 (102.4 mg, 0.65 mmol) afforded 123.5 mg (83% yield, 2 steps) of I-176 as a white solid. ESI-MS found 229.2, C₁₀H₁₇N₂O₄ (MH⁺) requires 229.1.

### Synthesis of I-180 is Depicted in Scheme 78

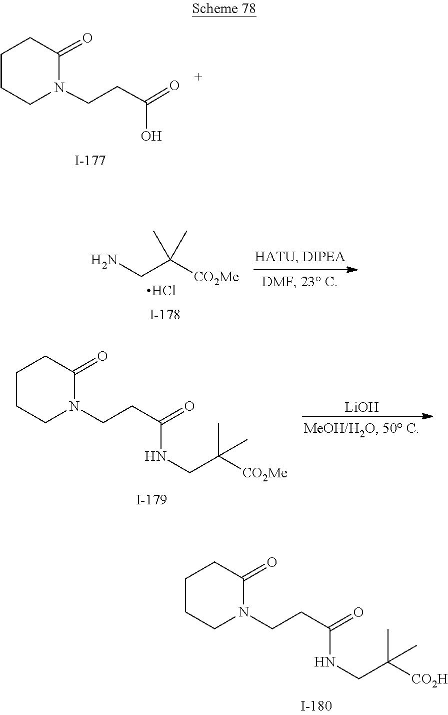

To a solution of carboxylic acid I-177 (79.1 mg, 0.46 mmol) and amine hydrochloride I-178 (77.5 mg, 1.0 equiv.) in DMF (1 mL) was added DIPEA (241 μL, 3.0 equiv.), followed by HATU (263.5 mg, 1.5 equiv.). The reaction mixture was stirred at ambient temperature for 3 hours, and then purified by preparative HPLC (H₂O/MeCN with 0.1% TFA) to afford the intermediate amide I-179 (190.2 mg) as a brown oil. This oil was redissolved in MeOH (3 mL). A solution of LiOH (22.1 mg, 2 equiv. based on carboxylic acid I-177) in H₂O (1 mL) was added. The reaction mixture was heated to 50° C. for 18 hours and then concentrated to remove MeOH. HCl (6M) was added to acidify the solution to pH<2, and the crude material was purified by preparative HPLC (H₂O/MeCN with 0.1% TFA) to afford I-180 (110.4 mg, 88% yield over 2 steps) as a colorless oil. ESI-MS found 271.2, C₁₃H₂₃N₂O₄ (MH⁺) requires 271.2.

### Synthesis of I-183 is Depicted in Scheme 79

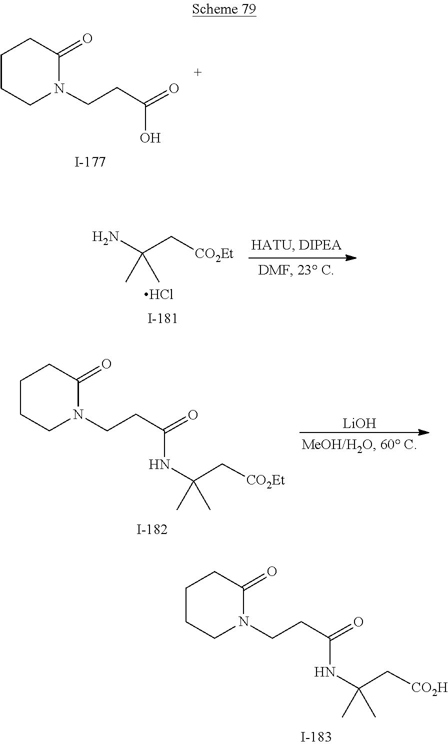

Using a procedure similar to that employed for the synthesis of I-180, carboxylic acid I-177 (77 mg, 0.45 mmol) afforded 62.4 mg (51% yield, 2 steps) of I-183 as a colorless oil. ESI-MS found 271.2, C₁₃H₂₃N₂O₄ (MH⁺) requires 271.2.

### Synthesis of I-184 is Depicted in Scheme 80

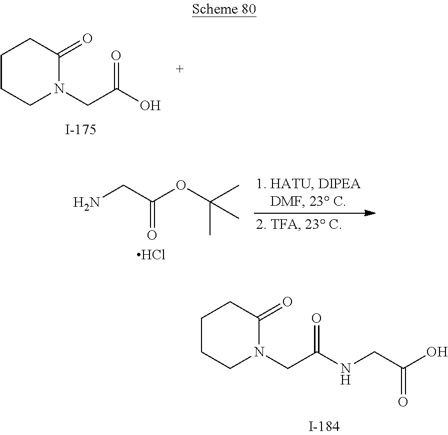

To a solution of carboxylic acid I-175 (72 mg, 0.46 mmol) and glycine tert-butyl ester hydrochloride (76.8 mg, 1.0 equiv.) in DMF (1.1 mL) was added DIPEA (240 μL, 3.0 equiv.), followed by HATU (261.5 mg, 1.5 equiv.). The reaction mixture was stirred at ambient temperature for 21 hours, then diluted with H₂O (1 mL), and purified by preparative HPLC (H₂O/MeCN with 0.1% AcOH) to afford the amide coupling product. TFA (5 mL) was added to this amide, and the reaction mixture was maintained at ambient temperature for 2 hours. The reaction mixture was then concentrated and purified by preparative HPLC (H₂O/MeCN with 0.1% TFA) to afford I-184 (84 mg, 86% yield over 2 steps) as a colorless oil. ESI-MS found 213.1 C₉H₁₃N₂O₄ (M−H) requires 213.1.

### Synthesis of I-185 is Depicted in Scheme 81

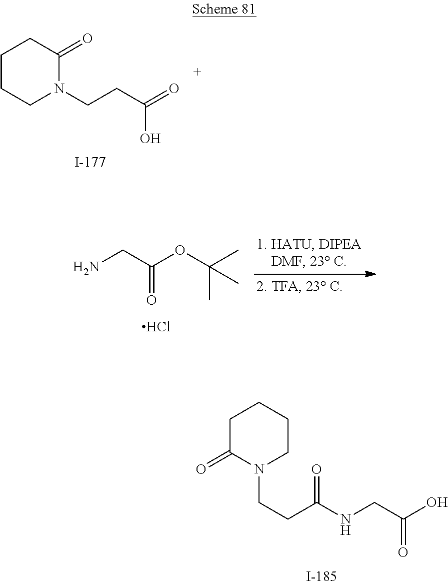

Using a procedure similar to that employed for the synthesis of I-184, carboxylic acid I-177 (72.4 mg, 0.42 mmol) afforded 104.1 mg (quant., 2 steps) of I-185 as a colorless oil. ESI-MS found 229.2 C₁₀H₁₇N₂O₄ (MH⁺) requires 229.1.

### Synthesis of I-187 is Depicted in Scheme 82

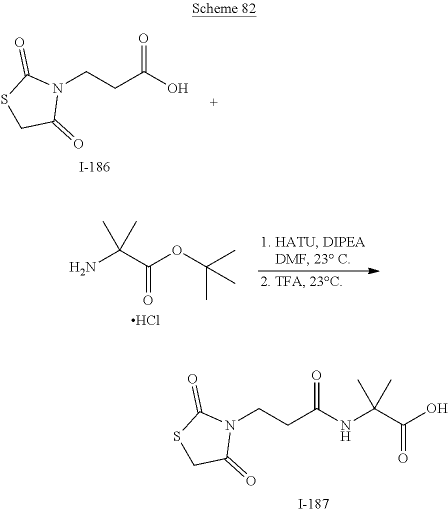 [SMILES: O=C(O)CCN1C(=O)CSC1=O ; CC(C)(C)OC(=O)C(C)(C)N ; Cl ; CC(C)(NC(=O)CCN1C(=O)CSC1=O)C(=O)O]

To a solution of carboxylic acid I-186 (96.4 mg, 0.51 mmol) and □-aminoisobutyric acid tert-butyl ester hydrochloride (119.7 mg, 1.2 equiv.) in DMF (2 mL) was added DIPEA (178 μL, 2.0 equiv.), followed by HATU (290.6 mg, 1.5 equiv.). The reaction mixture was stirred at ambient temperature for 3 hours, then concentrated. TFA (5 mL) was added to the residue. The reaction mixture was stirred at ambient temperature for 1 hour and then concentrated under reduced pressure. The residue was purified by preparative HPLC (H₂O/MeCN with 0.1% TFA) to afford 74 mg (53% yield, 2 steps) of I-187 as a white solid. ESI-MS found 273.1, C₁₀H₁₃N₂O₅S (M−H)⁻ requires 273.1.

### Synthesis of I-188 is Depicted in Scheme 83

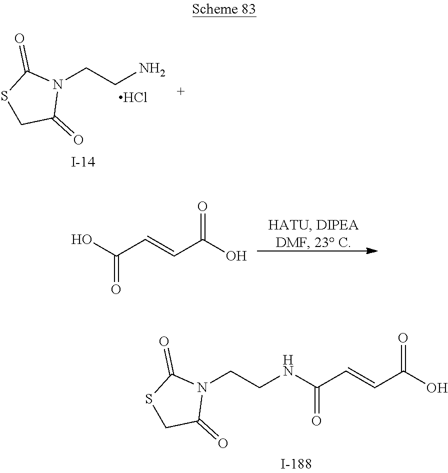

To a solution of amine hydrochloride I-14 (46.8 mg, 0.24 mmol) and fumaric acid (55.2 mg, 2.0 equiv.) in DMF (1.2 mL) was added DIPEA (124 μL, 3.0 equiv.), followed by HATU (135.7 mg, 1.5 equiv.). The reaction mixture was stirred at ambient temperature for 3.5 hours, then diluted with H₂O (0.3 mL), and purified by preparative HPLC (H₂O/MeCN with 0.1% TFA) to afford 45 mg (73% yield) of I-188 as a white solid. ESI-MS found 257.1, C₉H3N₂O₅S (M−H⁺) requires 257.1.

### Synthesis of I-191 is Depicted in Scheme 84

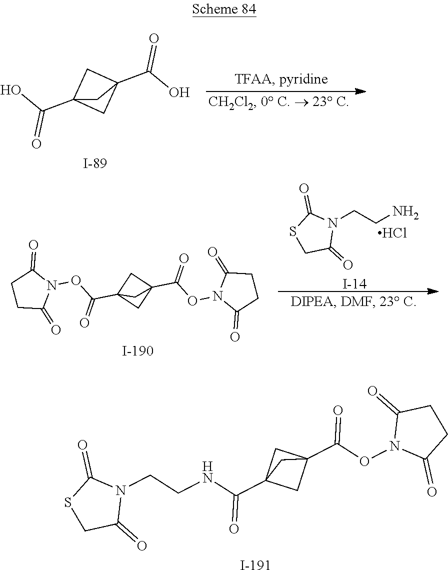

To a solution of dicarboxylic acid I-189 (101.5 mg, 0.65 mmol) and HOSu (149.6 mg, 2.0 equiv.) in DCM (4 mL) was added pyridine (209 μL, 4.0 equiv.). The solution was cooled to 0-5° C. and TFAA (183 μL, 2.0 equiv.) was added. The reaction mixture was allowed to warm to ambient temperature, stirred for 16 hours, and then concentrated. EtOH (10 mL) was added and the precipitated solids were collected by filtration and dried in vacuo to afford 218 mg of crude diester I-190, which was taken forward to the next step without further purification.

To a solution of amine salt I-14 (42.6 mg, 0.22 mmol) and diester I-190 (151.7 mg ca. 2.0 equiv.) in DMF (1.3 mL) was added DIPEA (76 μL, 2.0 equiv.). The reaction mixture was agitated for 16 hours and then purified by preparative HPLC (H₂O/MeCN with 0.1% TFA) to afford 29 mg (34% yield) of I-191 as a colorless oil.

### Synthesis of I-194 is Depicted in Scheme 85

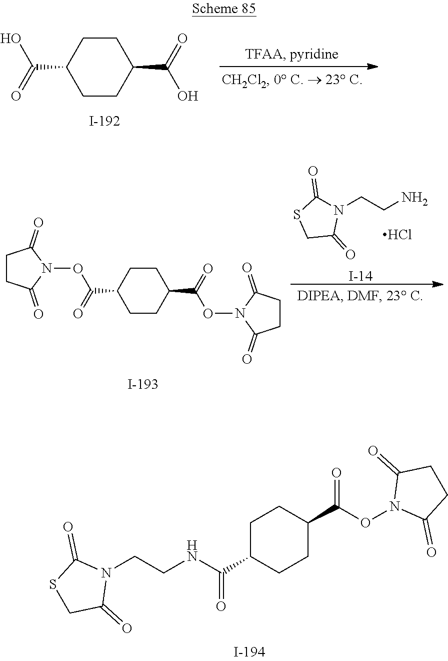

Diester I-193 and activated ester I-194 were prepared as described for I-191. Yield: 13 mg (10% yield) of I-194 as a colorless oil.

### Synthesis of I-197 is Depicted in Scheme 86

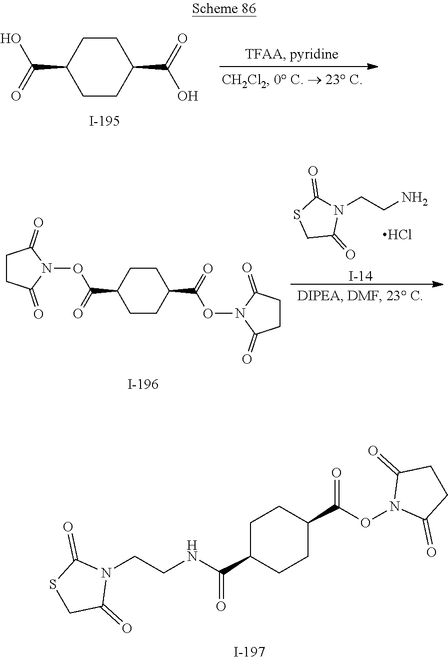

Diester I-196 and activated ester I-194 were prepared as described for I-191. Yield: 33.8 mg (31% yield) of I-197 as a white solid.

### Synthesis of I-200 is Depicted in Scheme 87

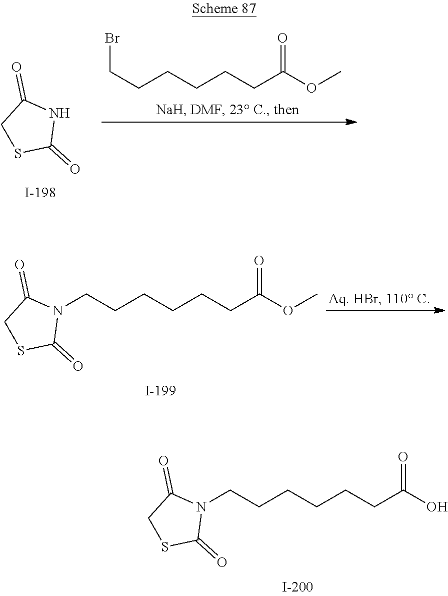 [SMILES: O=C1CSC(=O)N1 ; COC(=O)CCCCCCBr ; COC(=O)CCCCCCN1C(=O)CSC1=O ; O=C(O)CCCCCCN1C(=O)CSC1=O]

To a solution of thiazolidinedione I-198 (1.0 g, 8.55 mmol) in anhydrous DMF (10 mL) was added NaH (376.7 mg, 9.41 mmol, 1.1 equiv.), and the reaction mixture was stirred for 30 minutes at ambient temperature. Methyl-7-bromoheptanoate (2.09 g, 1.1 equiv.) was added dropwise, and the mixture was stirred at ambient temperature for 16 hours. The reaction was monitored by TLC. The reaction mixture was quenched with H₂O (20 mL) and extracted with EtOAc (3×20 mL). The combined organic extracts were concentrated and the residue obtained was purified by preparative TLC (Petroleum ether/EtOAc=1:1) to afford I-199 (900 mg, 41%) as a white solid.
TLC: (Petroleum ether/EtOAc=1:1), UV 254 nm
R_{f} (compound I-198)=0.4
R_{f} (compound I-199)=0.5

A solution of thiazolidinedione I-199 (900 mg, 3.47 mmol) in 40% aqueous HBr (10 mL) was refluxed at 110° C. for 4 hours. The reaction was monitored by TLC. Water (10 mL) was added and the reaction mixture was extracted with EtOAc (3×40 mL). The combined organic extracts were dried over Na₂SO₄, filtered, concentrated and the residue obtained was purified by preparative TLC (Petroleum ether/EtOAc=1:1) to afford I-200 (360 mg, 42%) as a white solid.
TLC: Petroleum ether/EtOAc=1:1, UV 254 nm
R_{f} (compound I-199)=0.5
R_{f} (I-200)=0.1

¹H NMR (400 MHz, d₆-DMSO) δ 11.92 (s, 1H), 4.17 (s, 2H), 3.47 (t, J=6.8 Hz, 2H), 2.18 (t, J=7.2 Hz, 2H), 1.57-1.37 (m, 4H), 1.34-1.12 (m, 4H).

### Synthesis of I-202 is Depicted in Scheme 88

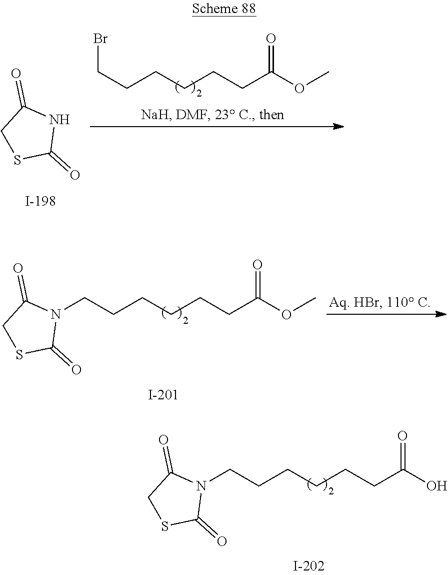

To a solution of thiazolidinedione I-198 (246 mg, 2.1 mmol) in anhydrous DMF (5 mL) was added NaH (53 mg, 1.1 equiv.) and the reaction mixture was stirred for 30 minutes at ambient temperature. Methyl-8-bromooctanoate (500 mg, 1.0 equiv.) was added dropwise. The mixture was stirred at ambient temperature for 16 hours. The reaction was monitored by TLC. The reaction mixture was quenched with H₂O (20 mL) and extracted with EtOAc (3×20 mL). The combined organic extracts were dried over Na₂SO₄ concentrated and the residue obtained was purified by preparative TLC (Petroleum ether/EtOAc=1:1) to afford intermediate ester I-201 (300 mg, 52%) as a white solid.
TLC: Petroleum ether/EtOAc=1:1, UV 254 nm
R_{f} (compound I-198)=0.4
R_{f} (compound I-201)=0.5

A solution of ester I-201 (300 mg, 1.1 mmol) in 40% aqueous HBr (5 mL) was refluxed at 110° C. for 4 hours. The reaction was monitored by TLC. Water (20 mL) was added and the reaction mixture was extracted with EtOAc (3×20 mL). The combined organic extracts were dried over Na₂SO₄, filtered, and concentrated to a residue, which was purified by preparative TLC (Petroleum Ether/EtOAc=1:1) to afford I-202 (144 mg, 51%) as a white solid.
TLC: Petroleum Ether/EtOAc=1:1, UV 254 nm
R_{f} (compound I-202)=0.5
R_{f} (I-202)=0.1

¹H NMR (400 MHz, d₆-DMSO) § 11.96 (s, 1H), 4.17 (s, 2H), 3.46 (t, J=6.8 Hz, 2H), 2.18 (t, J=7.6 Hz, 2H), 1.52-1.43 (m, 4H), 0.39-1.13 (m, 6H).

### Synthesis of I-204 is Depicted in Scheme 89

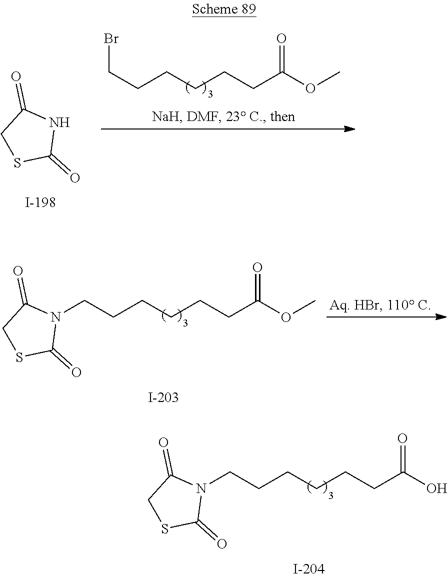

To a solution of thiazolidinedione I-198 (246 mg, 2.1 mmol) in anhydrous DMF (5 mL) was added NaH (53 mg, 1.1 equiv.) and the mixture was stirred for 30 minutes at ambient temperature. Methyl-9-bromononanoate (500 mg, 0.95 equiv.) was added dropwise and the mixture was stirred at ambient temperature for 4 hours. The reaction was monitored by TLC. The reaction mixture was quenched with H₂O (20 mL) and extracted with EtOAc (3×20 mL). The combined organic extracts were dried over Na₂SO₄ concentrated, and the residue obtained was purified by preparative TLC (Petroleum Ether/EtOAc=1:1) to afford ester I-203 (287 mg, 48%) as a white solid.
TLC: Petroleum Ether/EtOAc=1:1, UV 254 nm
R_{f} (compound I-198)=0.4
R_{f} (ester I-203)=0.5

A solution of ester I-203 (287 mg, 1.0 mmol) in 40% aqueous HBr (5 mL) was refluxed at 110° C. for 4 hours. The reaction was monitored by TLC. Water (10 mL) was added, and the reaction mixture was extracted with EtOAc (3×20 mL). The combined organic extracts were dried over Na₂SO₄, filtered, concentrated, and the residue obtained was purified by preparatory TLC (Petroleum Ether/EtOAc=1:1) to afford I-204 (122 mg 45%) as a white solid.
TLC: Petroleum Ether/EtOAc=1:1, UV 254 nm
R_{f} (compound I-203)=0.5
R_{f} (I-204)=0.1

¹H NMR (400 MHz, DMSO) δ 11.98 (s, 1H), 4.17 (s, 2H), 3.44 (t, J=6.0 Hz, 2H), 2.48 (t, J=6.0 Hz, 2H), 1.55-1.36 (m, 4H), 1.32-1.09 (m, 8H).

### Synthesis of I-209 is Depicted in Scheme 90

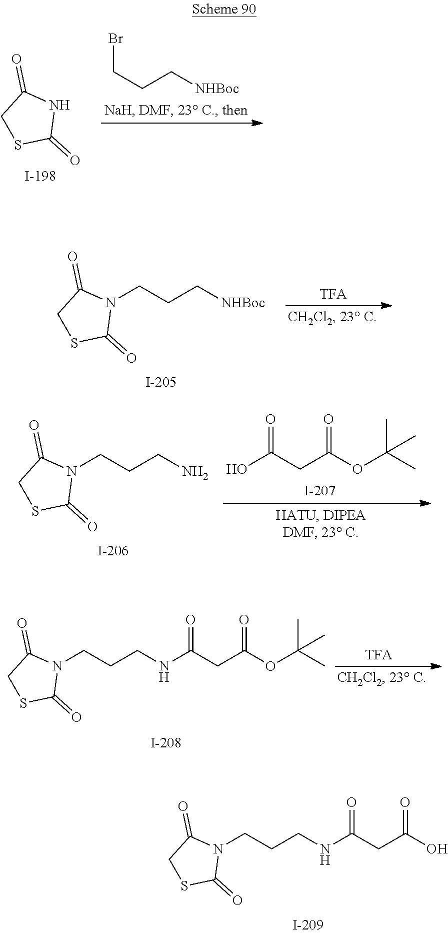

To a solution of thiazolidinedione I-198 (1.0 g, 8.55 mmol) in anhydrous DMF (10 mL) was added 60% NaH (376 mg, 1.1 equiv.) and the reaction mixture was stirred at ambient temperature for 1 hour. Tert-butyl (3-bromopropyl) carbamate (2.24 g, 1.1 equiv.) was added and the reaction mixture was stirred at ambient temperature for 3 hours. The reaction was monitored by TLC. The reaction mixture was quenched with H₂O (20 mL) and extracted with EtOAc (3×20 mL). The combined organic extracts were washed with brine, dried over Na₂SO₄, concentrated, and the residue obtained was purified by preparative TLC (Petroleum Ether/EtOAc=1:1) to afford carbamate I-205 (889 mg 38%) as a light yellow solid.
TLC: Petroleum Ether/EtOAc=1:1, UV 254 nm
R_{f} (compound I-198)=0.5
R_{f} (carbamate I-205)=0.6

To a solution of carbamate I-205 (500 mg, 1.82 mmol) in DCM (5 mL) was added TFA (0.5 mL). The reaction mixture was stirred at ambient temperature for 1 hour. The reaction was monitored by TLC. The reaction mixture was concentrated under reduced pressure to afford amine I-206 (315 mg, 99%) as a light yellow solid.
TLC: Petroleum Ether/EtOAc=1:1, UV 254 nm
R_{f} (compound I-205)=0.6
R_{f} (compound amine I-206)=0.1

A solution of amine I-206 (315 mg, 1.81 mmol), carboxylic acid I-207 (263 mg, 1.0 equiv.), HATU (688 mg, 1.1 equiv.), and DIPEA (851 mg, 4.0 equiv.) in anhydrous DMF (3 mL) was stirred at ambient temperature for 16 hours. The reaction was monitored by TLC. The reaction mixture was quenched with H₂O (5 mL) and extracted with EtOAc (3×5 mL). The combined organic extracts were dried over Na₂SO₄, and concentrated to a residue that was purified by preparative TLC (Petroleum Ether/EtOAc=1:1) to afford amide I-208 (250 mg, 48%) as a light yellow solid.
TLC: Petroleum Ether/EtOAc=1:1, UV 254 nm
R_{f} (amine I-206)=0.1
R_{f} (amide I-208)=0.6

To a solution of amide I-208 (250 mg, 0.79 mmol) in DCM (3 mL) was added TFA (0.3 mL). The mixture was stirred at ambient temperature for 4 hours. The reaction was monitored by TLC. The reaction mixture was concentrated to afford a residue, which was purified by preparative HPLC to yield acid I-209 (50 mg, 24%) as a white solid.
TLC: Petroleum Ether/EtOAc=1:1, UV 254 nm
R_{f} (amide I-208)=0.6
R_{f} (I-209)=0.1

¹H NMR (400 MHz, DMSO): δ 12.48 (br. s, 1H), 8.06 (s, 1H), 4.16 (s, 2H), 3.49 (t, J=6.8 Hz, 2H), 3.08 (s, 2H), 3.06-3.00 (m, 2H), 1.65-1.57 (m, 2H).

### Synthesis of I-213 is Depicted in Scheme 91

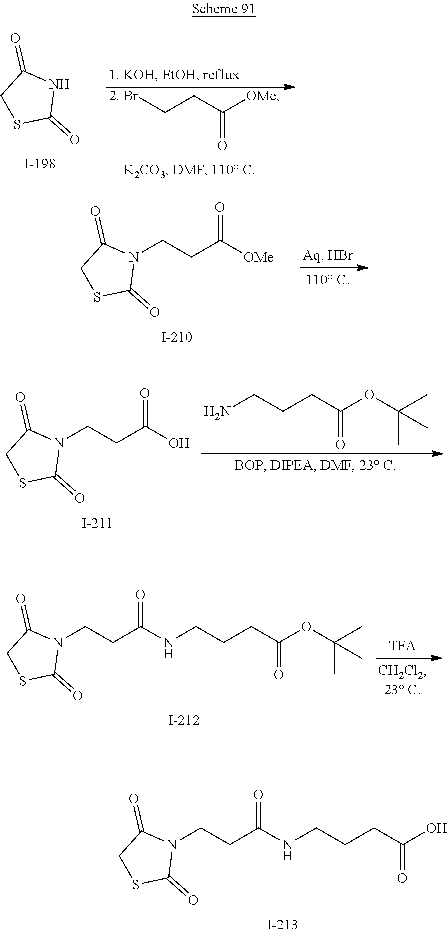

A solution of thiazolidinedione I-198 (5.0 g, 42.7 mmol) and KOH (2.63 g, 1.1 equiv.) in EtOH (50 mL) was refluxed for 4 hours. The reaction mixture was concentrated to give the crude potassium salt (6.62 g) as a white solid. To a solution of this salt (6.62 g 42.7 mmol) in DMF (70 mL) was added K₂CO₃ (25 g, 170.8 mmol, 4.0 equiv.) and methyl-3-bromopropionate (21.4 g, 3.0 equiv.). The reaction mixture was stirred at ambient temperature for 16 hours. The reaction was monitored by TLC and quenched with H₂O (100 mL), then extracted with EtOAc (3×80 mL). The combined organic extracts were washed with brine, dried over Na₂SO₄ and concentrated under reduced pressure to a residue, which was purified by flash chromatography on silica (Petroleum Ether/EtOAc=20:1-5:1) to afford ester I-210 (2.8 g, 32%) as a light yellow oil.
TLC: Petroleum Ether/EtOAc=1:1, UV 254 nm
R_{f} (compound I-198)=0.1
R_{f} (compound I-210)=0.5

A solution of ester I-210 (3.16 g, 15.56 mmol) in 45% HBr (25 mL) was refluxed at 110° C. for 4 hours. The reaction was monitored by TLC. Water (25 mL) was added and the reaction mixture was extracted with EtOAc (3×20 mL). The combined extracts were washed with brine, dried over Na₂SO₄, filtered, and concentrated to a residue, which was purified by preparative TLC (Petroleum Ether/EtOAc=1:1) to afford carboxylic acid I-211 (2.73 g, 93%) as a white solid.
TLC: Petroleum Ether/EtOAc=1:1, UV 254 nm
R_{f} (ester I-210)=0.5
R_{f} (carboxylic acid I-211)=0.1

A solution of carboxylic acid I-211 (719 mg, 3.8 mmol), gamma-aminobutyric acid tert-butyl ester (550 mg, 3.45 mmol, 1.0 equiv.), BOP (2.52 g, 1.1 equiv.) and DIPEA (2.25 g, 4.0 equiv.) in anhydrous DMF (8 mL) was stirred at ambient temperature for 16 hours. The reaction was monitored by TLC. The reaction mixture was quenched with H₂O (10 mL) and extracted with EtOAc (3×10 mL). The combined organic extracts were dried over Na₂SO₄, concentrated and the residue obtained was purified by preparative TLC (Petroleum Ether/EtOAc=1:1) to afford amide I-212 (600 mg, 52%) as a light yellow solid.
TLC: Petroleum Ether/EtOAc=1:1, UV 254 nm
R_{f} (carboxylic acid I-211)=0.1
R_{f} (amide I-212)=0.5

To a solution of amide I-212 (300 mg, 0.91 mmol) in DCM (3 mL) was added TFA (0.3 mL). The reaction mixture was stirred at ambient temperature for 2 hours. The reaction was monitored by TLC. The reaction mixture was concentrated and the residue obtained was purified by preparative HPLC to afford I-213 (52 mg, 21%) as a white solid.
TLC: Petroleum Ether/EtOAc=1:1, UV 254 nm
R_{f} (carboxylic acid I-211)=0.5
R_{f} (I-213)=0.1

¹H NMR (400 MHz, DMSO) δ 12.05 (s, 1H), 7.98 (t, J=5.2 Hz, 1H), 4.14 (s, 2H), 3.66 (t, J=7.6 Hz, 2H), 3.02-2.96 (m, 2H), 2.31 (t, J=7.2 Hz, 2H), 2.20 (t, J=7.6 Hz, 2H), 1.61-1.53 (m, 2H).

### Synthesis of I-217 is Depicted in Scheme 92

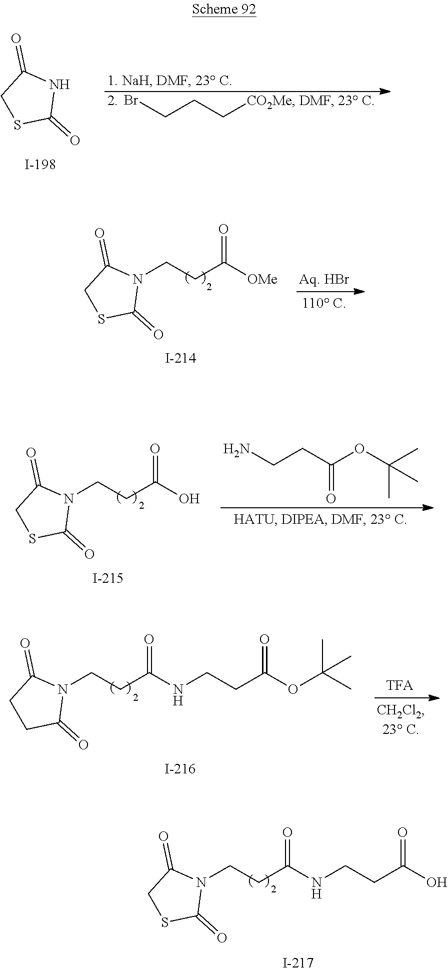

To a solution of thiazolidinedione I-198 (1.62 g, 13.8 mmol) in anhydrous DMF (15 mL) was added NaH (552 mg, 1.0 equiv.) and the reaction mixture was stirred for 10 min at ambient temperature. Methyl-4-bromobutanoate (2.5 g, 1.0 equiv.) was added dropwise, and the reaction mixture was stirred at ambient temperature for 4 hours. The reaction was monitored by TLC. H₂O (20 mL) was added and the mixture was extracted with EtOAc (3×20 mL). The combined organic extracts were washed with 5% HCl solution, dried over Na₂SO₄, and concentrated; the residue obtained was purified by preparative TLC (Petroleum Ether/EtOAc=2:1) to afford ester I-214 (1.8 g, 60% yield) as a colorless oil.
TLC: Petroleum Ether/EtOAc=1:1, UV 254 nm
R_{f} (compound I-198)=0.2
R_{f} (ester I-214)=0.5

A solution of ester I-214 (1.8 g, 8.29 mmol) in 40% HBr (15 mL) was refluxed at 110° C. for 4 hours. The reaction was monitored by TLC. Water (15 mL) was added, and the reaction was extracted with EtOAc (3×50 mL). The combined organic extracts were dried over Na₂SO₄, filtered, and concentrated; the residue obtained was purified by preparative TLC (EtOAc) to afford carboxylic acid I-215 (1.3 g, 77%) as a white solid.
TLC: Petroleum Ether/EtOAc=2:1, UV 254 nm
R_{f} (ester I-214)=0.6
R_{f} (carboxylic acid I-215)=0.3

A solution of carboxylic acid I-215 (200 mg, 1.0 mmol), DIPEA (387 mg, 3.0 equiv.), β-alanine-tert-butyl ester (143 mg, 1.0 equiv.) and HATU (564 mg, 1.5 equiv.) in anhydrous DMF (2 mL) was stirred at ambient temperature for 16 hours. The reaction was monitored by TLC. H₂O (10 mL) was added and the reaction mixture was extracted with DCM (3×10 mL). The combined organic extracts were dried over Na₂SO₄, and concentrated; the residue obtained was purified by preparative TLC (Petroleum Ether/EtOAc=1:2) to afford amide I-216 (150 mg, 46%) as a light yellow oil.
TLC: Petroleum Ether/EtOAc=1:1, UV 254 nm
R_{f} (carboxylic acid I-215)=0.2
R_{f} (amide I-216)=0.7

To a solution of amide I-216 (150 mg, 0.455 mmol) in DCM (2 mL) was added TFA (2 mL). The reaction mixture was stirred at ambient temperature for 2 hours. The reaction was monitored by LC-MS. The reaction mixture was concentrated, and the residue obtained was purified by preparative HPLC to afford I-217 (30 mg, 24%) as a colorless oil.

LC-MS: 275 (M+1)

¹H NMR (400 MHz, CD₃OD): δ 4.06 (s, 2H), 3.63 (t, J=6.4 Hz, 2H), 3.42-3.38 (m, 2H), 2.50 (t, J=7.6 Hz, 2H), 2.20 (t, J=7.2 Hz, 2H), 1.90-1.85 (m, 2H).

### Synthesis of I-221 is Depicted in Scheme 93

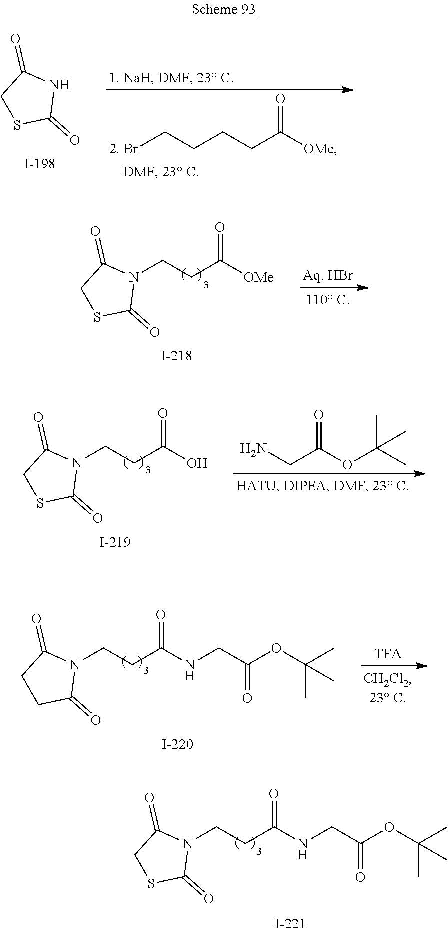

To a solution of thiazolidinedione I-198 (2 g, 17.1 mmol) in anhydrous DMF (25 mL) was added NaH (820 mg, 1.2 equiv.), and the reaction mixture was stirred for 10 minutes at ambient temperature. Methyl-5-bromopentanoate (3.66 g, 1.1 equiv.) was added dropwise to the reaction and the mixture was stirred at ambient temperature for 4 hours. The reaction was monitored by TLC. H₂O (40 mL) was added, and the reaction mixture was extracted with EtOAc (3×30 mL). The combined organic extracts were washed with 5% HCl solution, dried over MgSO₄, and concentrated. The residue obtained was purified by preparative TLC (Petroleum Ether/EtOAc=2:1) to afford ester I-218 (1.23 g, 31%) as a colorless oil.
TLC: Petroleum Ether/EtOAc=2:1, UV 254 nm
R_{f} (compound I-198)=0.2
R_{f} (ester I-218)=0.5

A solution of ester I-218 (1.23 g, 5.32 mmol) in 40% HBr (12 mL) was refluxed at 110° C. for 4 hours. The reaction was monitored by TLC. Water (12 mL) was added, and the reaction mixture was extracted with EtOAc (3×50 mL). The combined organic extracts were dried over MgSO₄, filtered, and concentrated; the residue obtained was purified by preparative TLC (EtOAc) to afford carboxylic acid I-219 (777 mg, 67%) as a white solid.
TLC: Petroleum Ether/EtOAc=2:1, UV 254 nm
R_{f} (ester I-218)=0.6
R_{f} (carboxylic acid I-219)=0.3

A solution of carboxylic acid I-219 (200 mg, 0.922 mmol), DIPEA (142 mg, 1.2 equiv.), glycine-tert-butyl ester (145 mg, 1.2 equiv.) and HATU (420 mg, 1.2 equiv.) in anhydrous DMF (5 mL) was stirred at ambient temperature for 4 hours. The reaction was monitored by TLC. H₂O (10 mL) was added and the reaction mixture was extracted with DCM (3×10 mL). The combined organic extracts were dried over Na₂SO₄, and concentrated. The residue obtained was purified by preparative TLC (Petroleum Ether/EtOAc=1:2) to give amide I-220 (200 mg, 66%) as a yellow oil.
TLC: Petroleum Ether/EtOAc=1:1, UV 254 nm
R_{f} (carboxylic acid I-219)=0.2
R_{f} (amide I-220)=0.7

To a solution of amide I-220 (200 mg, 0.606 mmol) in DCM (2 mL) was added TFA (2 mL). The reaction mixture was stirred at ambient temperature for 2 hours. The reaction was monitored by LC-MS. The reaction mixture was concentrated, and the residue obtained was purified by preparative HPLC to give I-221 (77 mg, 46%) as a white solid.

LC-MS: 275 (M+1)

¹H NMR (400 MHz, CD₃OD): δ 4.07 (s, 2H), 3.88 (s, 2H), 3.63 (t, J=6.4 Hz, 2H), 2.29 (t, J=6.4 Hz, 2H), 1.63-1.30 (m, 4H).

### The Synthesis of Compound I-225 Involved 4 Steps as Depicted in the Following Scheme 94

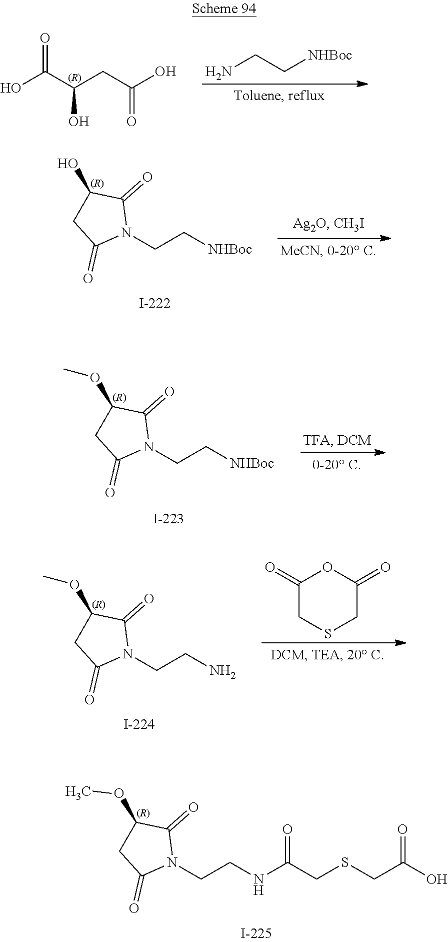

The chemical structure of the target compound was confirmed by ¹H NMR and LC-MS with the purity >95% (HPLC 214 & 254 nm).

### Step 1: tert-butyl (R)-(2-(3-hydroxy-2,5-dioxopyrrolidin-1-yl)ethyl)carbamate (I-222)

A mixture of (R)-2-hydroxysuccinic acid (cas: 636-61-3, 2 g, 14.92 mmol), tert-butyl (2-aminoethyl)carbamate (cas: 57260-73-8, 2 g, 1.0 equiv.) in toluene (25 mL) was refluxed at 130° C. for 2 hours. The reaction mixture was then cooled to ambient temperature and concentrated to give the crude product. The crude product was purified by silica gel chromatography to provide I-222 as a pale yellow oil (0.624 g, 17% yield).MS (ESI, pos. ion) m/z: 281 (M+Na).

### Step 2: tert-butyl (R)-(2-(3-methoxy-2,5-dioxopyrrolidin-1-yl)ethyl)carbamate (I-223)

To a solution of tert-butyl (R)-(2-(3-hydroxy-2,5-dioxopyrrolidin-1-yl)ethyl)carbamate (0.624 g, 2.42 mmol) in anhydrous MeCN (10 mL) at 0-5° C. was added Ag₂O (1.12 g, 2.0 equiv.) under N₂. The reaction mixture was then stirred for 5 minutes and MeI (0.309 g, 0.9 equiv.) was added to the reaction. The reaction mixture was allowed to warm to ambient temperature and stirred for another 10 hours, then filtered. The filtrate was concentrated in vacuo to give the crude product, which was purified by preparative TLC (Petroleum ether:EtOAc=1:2) to provide I-223 as a pale oil (0.2 g, 30% yield). MS (ESI, pos. ion) m/z: 295 (M+Na).

### Step 3: (R)-1-(2-Aminoethyl)-3-Methoxypyrrolidine-2,5-Dione (I-224)

A solution of tert-butyl (R)-(2-(3-methoxy-2,5-dioxopyrrolidin-1-yl)ethyl)carbamate (200 mg, 0.735 mmol) in TFA/DCM (1:3 v/v, 8 mL) was stirred at 25° C. for 1 hour. When LC-MS analysis indicated that the reaction was complete, the reaction mixture was concentrated under reduced pressure to afford the crude amine TFA salt I-224 (390 mg) as a pale oil, which was used directly in next step. MS (ESI, pos. ion) m/z: 173 (M+1)

### Step 4: (R)-2-((2-((2-(3-methoxy-2,5-dioxopyrrolidin-1-yl)ethyl)amino)-2-oxoethyl)thio)acetic acid (I-225)

To a solution of (R)-1-(2-aminoethyl)-3-methoxypyrrolidine-2,5-dione (390 mg 1.37 mmol) and thiodiglycolic anhydride (217 mg, 1.2 equiv.) in 5 mL of DCM was added TEA (166 mg, 1.2 equiv.). The reaction mixture was stirred for 2 hours, and then concentrated in vacuo to give the crude product. The crude product was purified by preparative HPLC (Mobile Phase: MeCN/H₂O (0.1% TFA)) to afford I-225 (130 mg 19% yield) as a pale oil. MS (ESI, pos. ion) m/z: 305 (M+1). ¹H NMR (400 MHz, DMSO) δ 8.14 (m, 1H), 4.31 (m, 1H), 3.47-3.39 (m, 5H), 3.33 (s, 2H), 3.28-3.17 (m, 2H), 3.14 (s, 2H), 2.96 (m, 1H), 2.55 (m, 1H).

### The Synthesis of Compound I-229 Involved 4 Steps as Depicted in the Following Scheme 95

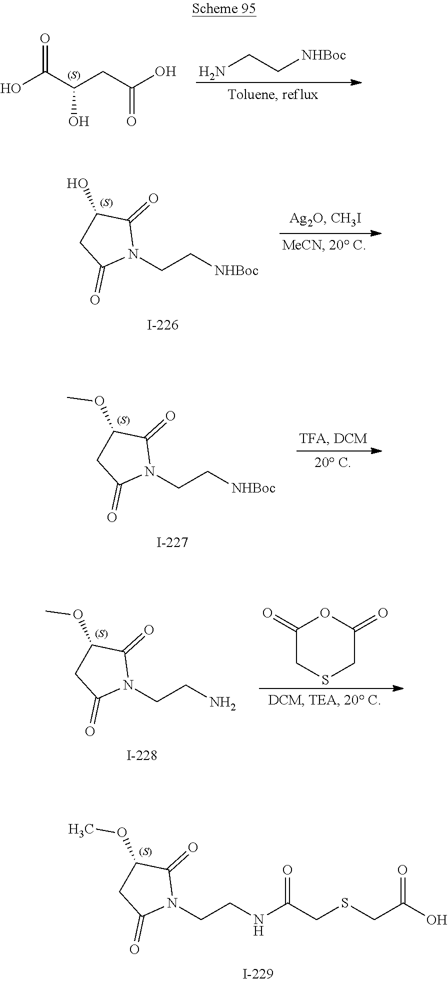

### (S)-2-((2-((2-(3-methoxy-2,5-dioxopyrrolidin-1-yl)ethyl)amino)-2-oxoethyl)thio)acetic acid (I-229)

The synthetic route for I-229 was similar with I-225 and commenced with (S)-2-hydroxysuccinic acid (cas: 97-67-6). I-229 (pale oil, 120 mg, 15% yield) was isolated by preparative HPLC (Mobile Phase: MeCN/H₂O (0.1% TFA)). MS (ESI, pos. ion) m/z: 305 (M+1). ¹H NMR (400 MHz, CDCl₃) δ 7.41 (s, 1H), 5.68 (s, 1H), 4.28 (m, 1H), 3.71 (m, 2H), 3.60 (s, 3H), 3.56-3.50 (m, 2H), 3.33 (m, 4H), 3.05 (m, 1H), 2.70-2.62 (m, 1H).

### E-149 (from (R*)-I-234) and E-150 (from (S*)-I-234)

### The Syntheses of Both Enantiomers of I-234 Involved 5 Steps as Depicted in Scheme 96

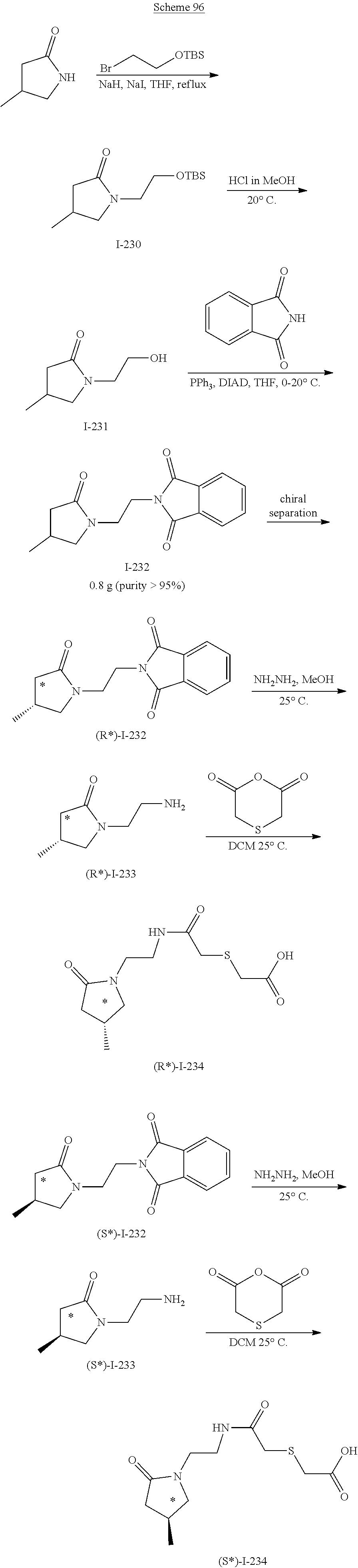

### Step 1: 1-(2-((tert-butyldimethylsilyl)oxy)ethyl)-4-methylpyrrolidin-2-one (I-230)

(2-bromoethoxy)(tert-butyl)dimethylsilane (cas: 86864-60-0, 3.18 g, 15 mmol) in 20 mL of THF was added dropwise to an ice-cooled suspension of 4-methylpyrrolidin-2-one (cas: 2996-58-9, 1 g, 1.0 equiv.), NaI (1.5 g, 1.0 equiv.) and NaH (2.4 g, 6.0 equiv.) in THF (50 mL) with stirring under N₂. After 2 hours, the ice bath was removed and the reaction mixture was heated to reflux overnight. The reaction was quenched with H₂O (50 mL). After removal of the solvent under reduced pressure, the residue was partitioned between EtOAc and H₂O. The aqueous layer was extracted with EtOAc (3×20 mL). The combined organic extracts were dried over MgSO₄, concentrated and the residue obtained was purified by silica gel chromatography to afford 360 mg (14%) of I-230 as a pale oil. MS (ESI, pos. ion) m/z: 258 (M+1).

### Step 2: 1-(2-hydroxyethyl)-4-methylpyrrolidin-2-one (I-231)

To 1-(2-((tert-butyldimethylsilyl)oxy)ethyl)-methylpyrrolidin-2-one I-230 (550 mg, 2.14 mmol) in MeOH (5 ml) was added 10 mL of HCl (4 M in MeOH). After 10 min, MeOH was removed under reduced pressure to afford 300 mg of the crude alcohol I-231 as a light yellow oil, which was taken forward to the next step without purification.

### Step 3: 2-(2-(4-methyl-2-oxopyrrolidin-1-yl)ethyl)isoindoline-1,3-dione (I-232)

To a stirred solution of 1-(2-hydroxyethyl)-4-methylpyrrolidin-2-one I-231 (96 mg, 0.66 mmol), phthalimide (cas: 85-41-6, 105 mg, 1.0 equiv.) and triphenylphosphine (260 mg, 1.0 equiv.) in anhydrous THF (10 mL) was added a solution of DIAD (400 mg 3.0 equiv.) in anhydrous THF (2 mL) dropwise at 0° C. The reaction mixture was allowed to warm to ambient temperature and stirred for 8 hours. The reaction was monitored by LC-MS. Upon completion of the reaction, the reaction mixture was filtered. The filtrate was concentrated, and the resulting residue was purified by silica gel chromatography (10-80% EtOAc in hexanes) to give 145 mg of I-232 (60% purity) as light oil. MS (ESI, pos. ion) m/z: 273.1 (M+1).

The racemic mixture I-232 was subjected to chromatographic separation using a chiral column to afford the two enantiomers (CHIRALPAK AD column, (Hexane/EtOH=50/50 (v/v)), but their absolute configuration was not established.

### Step 4: 1-(2-aminoethyl)-4-methylpyrrolidin-2-one (I-233)

To a stirred solution of each enantiomer of 2-(2-(4-methyl-2-oxopyrrolidin-1-yl)ethyl)isoindoline-1,3-dione, I-232 (145 mg, 0.53 mmol) in MeOH (10 mL) was added hydrazine monohydrate (40 mg, 1.5 equiv.). The reaction mixture was stirred at ambient temperature for 18 hours and then filtered. The filtrate was concentrated. The residue was dissolved in DCM, and the precipitate was again filtered off. After concentration of the filtrate, the 72 mg (78%) of crude amine I-233 was used in the next step without purification. MS (ESI, pos. ion) m/z: 143.1 (M+1).

### Step 5: 2-((2-((2-(4-methyl-2-oxopyrrolidin-1-yl)ethyl)amino)-2-oxoethyl)thio) acetic acid (I-234)

To a stirred solution of each enantiomer of 1-(2-aminoethyl)-4-methylpyrrolidin-2-one, I-233 (144 mg, 1.06 mmol) in DCM (10 mL) at ambient temperature was added thiodiglycolic anhydride (cas: 3261-87-8, 210 mg, 1.6 mmol), and the resulting solution was stirred at ambient temperature for 1 hour. The reaction mixture was concentrated and purified by preparative HPLC to give 150 mg (55%) of I-234 as a light oil. MS (ESI, pos. ion) m/z: 275.1 (M+1).

(R*)-I-234: ¹H NMR (400 MHz, CDCl₃) δ 7.30 (s, 1H), 3.71-3.61 (m, 1H), 3.58-3.39 (m, 4H), 3.36 (s, 2H), 3.20 (d, 2H), 3.10 (dd, 1H), 2.65 (dd, 1H), 2.49 (td, 1H), 2.13 (dd, 1H), 1.14 (s, 3H).

(S*)-I-234: ¹H NMR (400 MHz, CDCl₃) δ 7.35 (s, 1H), 3.71-3.61 (m, 1H), 3.58-3.39 (m, 4H), 3.36 (s, 2H), 3.20 (d, 2H), 3.10 (dd, 1H), 2.65 (dd, 1H), 2.49 (td, 1H), 2.13 (dd, 1H), 1.12 (s, 3H).

### The Synthesis of I-240 Involved 6 Steps as Depicted in the Following Scheme 97

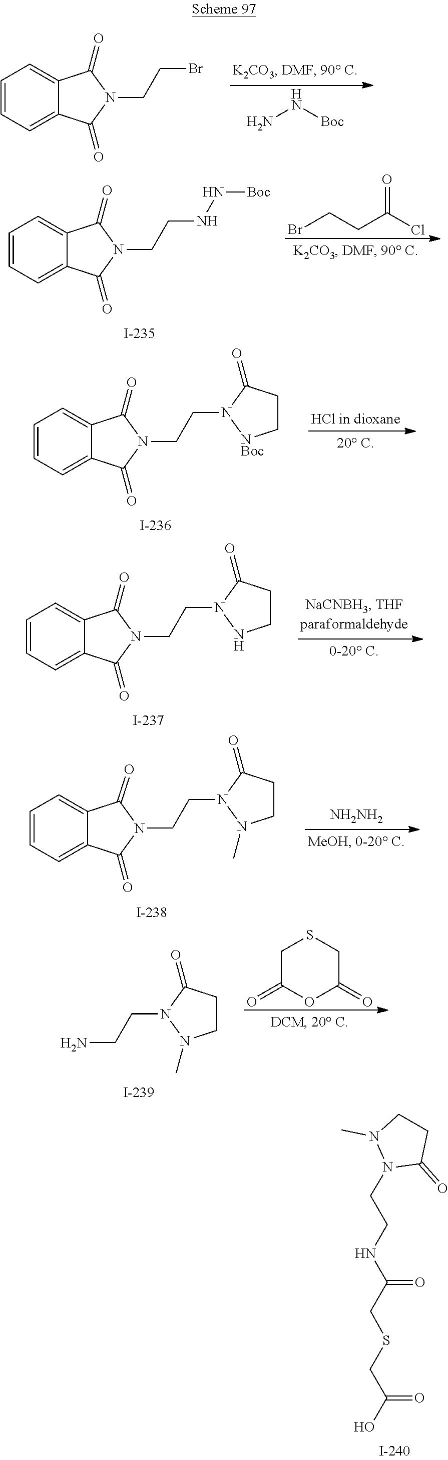

### Step 1: tert-butyl 2-(2-(1,3-dioxoisoindolin-2-yl)ethyl) hydrazine-1-carboxylate (I-235)

To a solution of 2-(2-bromoethyl)isoindoline-1,3-dione (cas: 574-98-1, 20 g, 79 mmol) and tert-butyl hydrazinecarboxylate (cas: 870-46-2, 16 g, 1.5 equiv.) in DMF (100 mL) was added K₂CO₃ (28 g, 2 equiv.). The solution was heated to 90° C. and stirred for 12 hours. Upon completion of the reaction, the reaction was quenched with water (100 mL) and extracted with EtOAc (3×100 mL). After drying with Na₂SO₄, the combined organic extracts were concentrated, and the crude product was purified by silica gel chromatography to provide 5 g of I-235 as a pale oil (21% yield). MS (ESI, pos. ion) m/z: 328.2 (M+23).

### Step 2: tert-butyl 2-(2-(1,3-dioxoisoindolin-2-yl)ethyl)-3-oxopyrazolidine-1-carboxylate (I-236)

A solution of tert-butyl 2-(2-(1,3-dioxoisoindolin-2-yl)ethyl) hydrazine-1-carboxylate (5 g, 16.4 mmol), 3-bromopropanoyl chloride (3.6 g, 1.5 equiv.) and K₂CO₃ (37 g, 2 equiv.) in DMF (80 mL) was heated to 90° C. for 16 hours. Upon completion of the reaction, the reaction was quenched with water (80 mL) and extracted with EtOAc (3×80 mL). After drying with Na₂SO₄, the combined organic extracts were concentrated, and the crude product was purified by silica gel chromatography to afford 3.2 g (54% yield) of I-236 as a pale oil. MS (ESI, pos. ion) m/z: 382.2 (M+23).

### Step 3: 2-(2-(5-oxopyrazolidin-1-yl)ethyl)isoindoline-1,3-dione I-237)

A solution of tert-butyl 2-(2-(1,3-dioxoisoindolin-2-yl)ethyl)-3-oxopyrazolidine-1-carboxylate (3.2 g, 8.9 mmol) in HCl (4M in dioxane, 15 mL) was stirred at ambient temperature for 0.5 hours. Upon completion of the reaction, the reaction mixture was concentrated to afford the crude pyrazolone I-237 (3.5 g) as a white solid, which was used directly in the next step. MS (ESI, pos. ion) m/z: 260.2 (M+1).

### Step 4:2-(2-(2-methyl-5-oxopyrazolidin-1-yl)ethyl)isoindoline-1,3-dione (I-238)

To a solution of 2-(2-(5-oxopyrazolidin-1-yl)ethyl)isoindoline-1,3-dione (3.5 g, ca. 8.9 mmol) and paraformaldehyde (2.7 g, 10 equiv.) in THF was added 2 drops of AcOH. After 1 hour, NaCNBH₃ (cas: 25895-60-7, 2.8 g, 5 equiv.) was added to the reaction mixture at 0° C. The reaction mixture was stirred for 12 hours, then quenched with water (30 mL), and extracted with DCM:MeOH (10:1) (3×40 mL). The combined organic extracts were dried over Na₂SO₄, and concentrated to a residue which was purified by silica gel chromatography to afford I-238 (0.6 g, 24% yield over 2 steps) as a yellow solid. MS (ESI, pos. ion) m/z: 274.2 (M+1).

### Step 5:2-(2-aminoethyl)-1-methylpyrazolidin-3-one (I-239)

To a solution of 2-(2-(2-methyl-5-oxopyrazolidin-1-yl)ethyl)isoindoline-1,3-dione (600 mg, 2.2 mmol) in MeOH (50 mL) at 0-5° C. was added NH₂NH₂ (85% in H₂O, 1.5 mL, 2.5 equiv.). The reaction mixture was allowed to warm to ambient temperature, stirred for 12 hours, and then filtered. The filtrate was concentrated to afford the crude amine I-239 (0.4 g) as a pale oil, which was used directly for the next step. MS (ESI, pos. ion) m/z: 144.2 (M+23).

### Step 6: 2-((2-((2-(2-methyl-5-oxopyrazolidin-1-yl)ethyl)amino)-2-oxoethyl)thio)acetic acid (I-240)

A solution of 2-(2-aminoethyl)-1-methylpyrazolidin-3-one (400 mg, 2.8 mmol) and thiodiglycolic anhydride (990 mg, 2.5 equiv.) in 10 mL of DCM was stirred for 0.5 hours and then concentrated in vacuo to afford the crude product. Half of this crude product was purified by preparative HPLC (Mobile Phase: MeCN/H₂O with 0.1% TFA), gradient: 5-15% MeCN) to afford 56 mg (15% yield) of I-240 as a white solid. MS (ESI, pos. ion) m/z: 276.2 (M+1). ¹H NMR (400 MHz, D2O) δ 3.75-3.70 (m, 2H), 3.38-3.34 (m, 4H), 3.29 (s, 2H), 3.25 (s, 2H), 3.02-2.85 (m, 1H), 2.81-2.77 (m, 3H), 2.55-2.42 (m, 1H).

### The Synthesis of I-243 Involved 5 Steps as Depicted in the Following Scheme 98

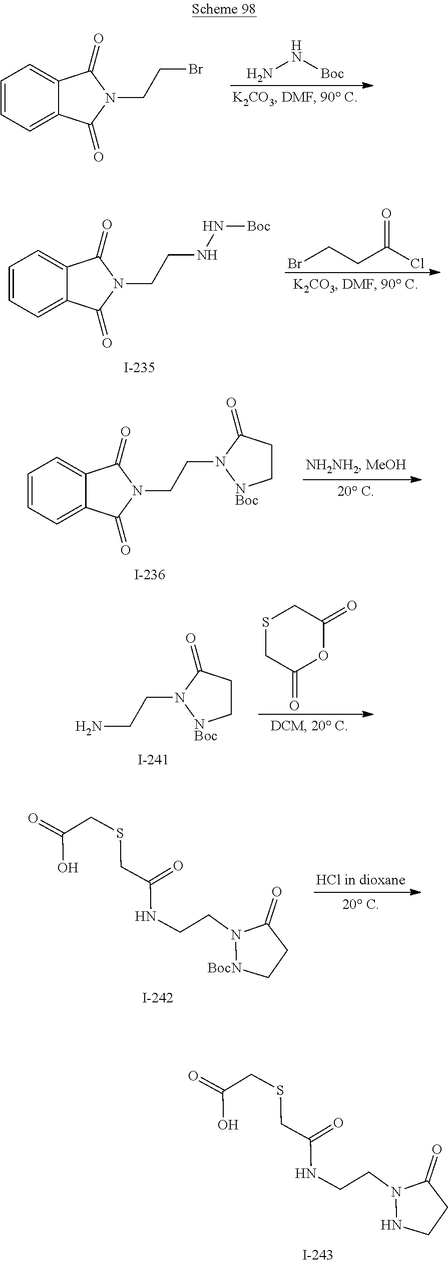

### Step 3: Tert-Butyl 2-(2-Aminoethyl)-3-Oxopyrazolidine-1-Carboxylate (I-241)

To a solution of tert-butyl 2-(2-(1,3-dioxoisoindolin-2-yl)ethyl)-3-oxopyrazolidine-1-carboxylate, I-236 (2 g, 5.5 mmol) in MeOH (50 mL) at 0-5° C. was added NH₂NH₂ (85% in H₂O, 1 mL, 2.5 equiv.). The reaction mixture was allowed to warm to ambient temperature, stirred for 12 hours, and then filtered. The filtrate was concentrated under reduced pressure to afford the crude amine I-241 (0.7 g) as a pale oil, which was used directly for next step. MS (ESI, pos. ion) m/z: 252.2 (M+23).

### Step 4: 2-((2-((2-(2-(tert-butoxycarbonyl)-5-oxopyrazolidin-1-yl)ethyl)amino)-2-oxoethyl)thio)acetic acid (I-242)

A solution of tert-butyl 2-(2-aminoethyl)-3-oxopyrazolidine-1-carboxylate I-241 (700 mg, 3 mmol), thiodiglycolic anhydride (990 mg, 2.5 equiv.), and TEA (0.8 mL, 2 equiv.) in 10 mL of DCM was stirred for 0.5 hours. The reaction mixture was then concentrated in vacuo to afford the crude product as a pale oil. Half of this crude product was purified by preparative HPLC to afford 270 mg (49% yield) of I-242 as a white solid. MS (ESI, pos. ion) m/z: 384.2 (M+23).

### Step 5: 2-((2-oxo-2-((2-(5-oxopyrazolidin-1-yl)ethyl)amino)ethyl)thio)acetic acid (I-243)

A solution of 2-((2-((2-(2-(tert-butoxycarbonyl)-5-oxopyrazolidin-1-yl)ethyl)amino)-2-oxoethyl)thio)acetic acid I-242 (270 mg, 0.74 mmol) in HCl (4 M in dioxane) (10 mL) was stirred at 20° C. for 0.5 hours. The reaction mixture was then concentrated and the residue obtained was purified by preparative HPLC (MeCN/H₂O with 0.1% TFA), gradient: 10-20% MeCN) to afford 160 mg (81% yield) of I-243 as a pale oil. MS (ESI, pos, ion) m/z: 262.2 (M+1). ¹H NMR (400 MHz, DMSO) δ 8.19 (s, 1H), 3.39 (dd, 2H), 3.34 (d, 4H), 3.25 (dd, 2H), 3.20 (s, 2H), 2.44 (t, 3H).

### The Synthesis of I-248 Involved 5 Steps as Depicted in the Following Scheme 99

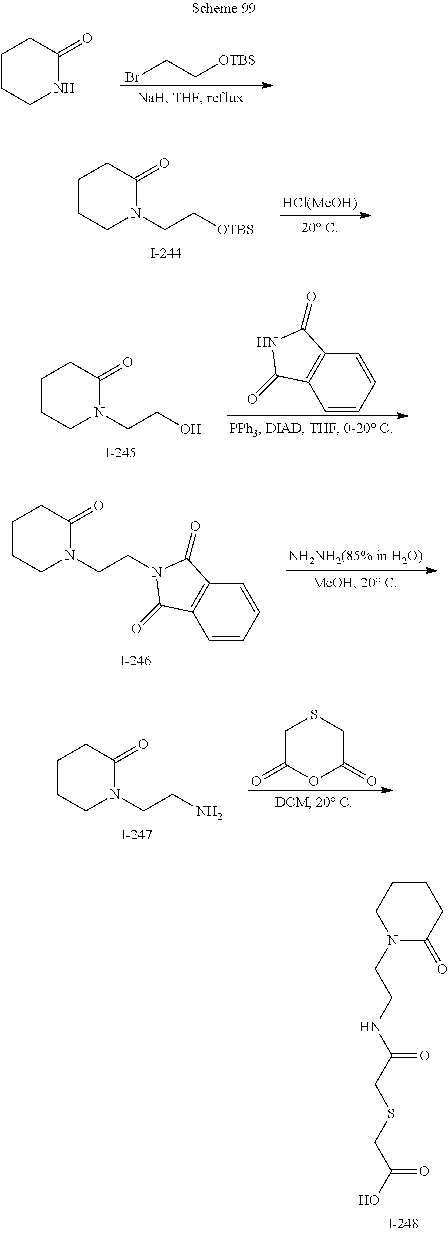

### Step 1: 1-(2-((tert-butyldimethylsilyl)oxy)ethyl)piperidin-2-one (I-244)

To a solution of piperidin-2-one (cas: 675-20-7, 6 g, 60 mmol) in THF (200 mL) at 0-5° C. was added NaH (4.8 g, 2 equiv., 60% dispersion in mineral oil) in portions under N₂. The reaction mixture was stirred for 0.5 hours. (2-bromoethoxy)(tert-butyl)dimethylsilane (cas: 86864-60-0, 4.9 g, 1.2 equiv.) was then added and the reaction mixture was heated to reflux for 6 hours. The reaction was then quenched with water (50 mL), and extracted with EtOAc (3×80 mL). The combined organic extracts were washed with water (50 mL), brine (50 mL), dried with Na₂SO₄, and concentrated under reduced pressure to a residue, which was purified by silica gel chromatography to afford I-244 as a pale oil (2 g, 13% yield). MS (ESI, pos. ion) m/z: 258 (M+1).

### Step 2: 1-(2-hydroxyethyl)piperidin-2-one (I-245)

To a solution of 1-(2-((tert-butyldimethylsilyl)oxy)ethyl)piperidin-2-one I-244 (2 g, 7.8 mmol) in MeOH (15 mL) at 0-5° C. was added HCl in MeOH (30% v/v, 10 mL) under N₂. The reaction mixture was stirred for 0.5 hours, then concentrated under reduced pressure to afford the crude alcohol I-245 as a pale oil (1.2 g), which was used directly for next step. MS (ESI, pos. ion) m/z: 144 (M+1).

### Step 3: 2-(2-(2-oxopiperidin-1-yl)ethyl)isoindoline-1,3-dione (I-246)

To a solution of 1-(2-hydroxyethyl)piperidin-2-one I-245 (1.2 g, 8.4 mmol) in THF (15 mL) at 0-5° C. was added phthalimide (1.85 g, 1.5 equiv.) and PPh₃ (3.9 g, 1.8 equiv.) and the reaction mixture was stirred for 0.5 hours under N₂. DIAD (3.4 g, 2.0 equiv.) was added and the reaction mixture was allowed to warm to ambient temperature and stirred for 2 hours. The reaction mixture was then filtered, and the filtrate was concentrated under reduced pressure to afford a pale oil, which was purified by silica gel chromatography to afford I-246 (purity: 60%, 0.7 g, 35% yield). MS (ESI, pos. ion) m/z: 273 (M+1).

### Step 4: 1-(2-aminoethyl)piperidin-2-one (I-247)

To a solution of 2-(2-(2-oxopiperidin-1-yl)ethyl)isoindoline-1,3-dione I-246 (0.7 g, 2.6 mmol) in MeOH (10 mL) at 0-5° C. was added NH₂NH₂ (85% in H₂O, 0.2 mL, 1.2 equiv.). The reaction mixture was allowed to warm to ambient temperature and stirred for 12 hours, then filtered, and the filtrate was concentrated under reduced pressure to afford the crude amine I-247 (0.4 g) as a pale oil, which was used directly for next step. MS (ESI, pos. ion) m/z: 143 (M+1).

### Step 5: 2-((2-oxo-2-((2-(2-oxopiperidin-1-yl)ethyl)amino)ethyl)thio)acetic acid (I-248)

A solution of 1-(2-aminoethyl)piperidin-2-one I-247 (400 mg, 2.8 mmol) and thiodiglycolic anhydride (924 mg, 2.5 equiv.) in 20 mL of DCM was stirred for 0.5 hours, and then concentrated in vacuo to give the crude product as a pale oil. The crude product was purified by preparative HPLC (Mobile Phase: MeCN/H₂O (0.1% formic acid), Gradient: 5-15% MeCN) to afford 200 mg of I-248 (26% yield) as a pale oil. MS (ESI, pos. ion) m/z: 275.2 (M+1). ¹H NMR (400 MHz, CDCl₃): δ 7.57 (s, 1H), 6.48 (s, 2H), 3.83-3.07 (m, 10H), 2.44 (s, 2H), 1.82 (s, 4H).

### The Synthesis of I-249 Involved 1 Step as Depicted in the Following Scheme 100

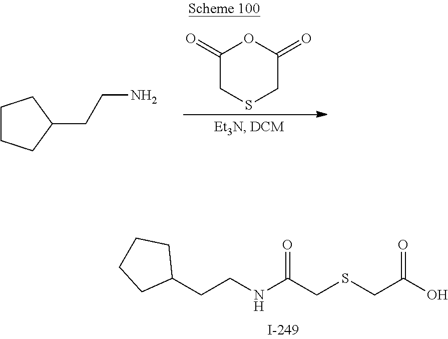 [SMILES: NCCC1CCCC1 ; O=C1CSCC(=O)O1 ; O=C(O)CSCC(=O)NCCC1CCCC1]

### Step 1: 2-((2-((2-cyclopentylethyl)amino)-2-oxoethyl)thio) acetic acid (I-249)

A solution of 2-cyclopentylethan-1-amine (cas: 5763-55-3, 200 mg, 1.8 mmol) and thiodiglycolic anhydride (594 mg, 2.5 equiv.) in 5 mL of DCM was stirred for 0.5 hours, and then concentrated in vacuo to afford a pale oil, which was purified by preparative HPLC (Mobile Phase: MeCN/H₂O (0.1% TFA), Gradient: 30-40% MeCN) to provide 250 mg (56% yield) of I-249 as a white solid. MS (ESI, pos. ion) m/z: 246.2 (M+1). ¹H NMR (400 MHz, CDCl₃): δ 6.64 (s, 1H), 3.47-3.22 (m, 6H), 1.79 (s, 3H), 1.68-1.47 (m, 6H), 1.11 (d, 2H).

### The Synthesis of Compound I-250 Involved 1 Step as Depicted in the Following Scheme 101

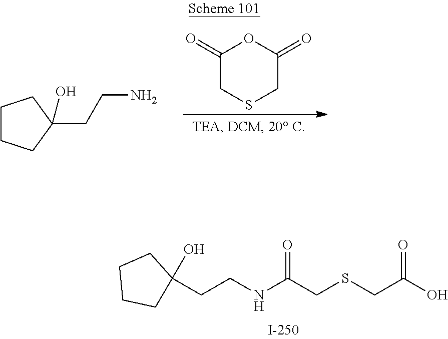 [SMILES: NCCC1(O)CCCC1 ; O=C1CSCC(=O)O1 ; O=C(O)CSCC(=O)NCCC1(O)CCCC1]

The chemical structure of the target compound was confirmed by ¹HNMR and LC-MS with the purity >95% (HPLC 214 & 254 nm).

### Step 1. 2-((2-((2-(1-hydroxycyclopentyl)ethyl)amino)-2-oxoethyl)thio) acetic acid (I-250)

To a solution of 1-(2-aminoethyl)cyclopentan-1-ol (cas: 859629-83-7, 0.1 g, 0.774 mmol) and thiodiglycolic anhydride (0.102 g, 1.0 equiv.) in DCM (5 mL) was added TEA (93 mg, 1.2 equiv.) and the reaction mixture was stirred for 4 hours at ambient temperature, then concentrated under reduced pressure to afford the crude product. The crude product was purified by preparative HPLC (Mobile Phase: MeCN/H₂O (0.1% TFA)) to afford I-250 (50 mg, 24% yield) as a pale oil. MS (ESI, pos. ion) m/z: 262.2 (M+1). ¹H NMR (400 MHz, DMSO): δ 12.63 (s, 1H), 7.97 (s, 1H), 4.09 (m, 1H), 3.35 (s, 2H), 3.20-3.12 (m, 4H), 1.75-1.35 (m, 10H).

### The Synthesis of I-257 Involved 7 Steps as Depicted in the Following Scheme 102

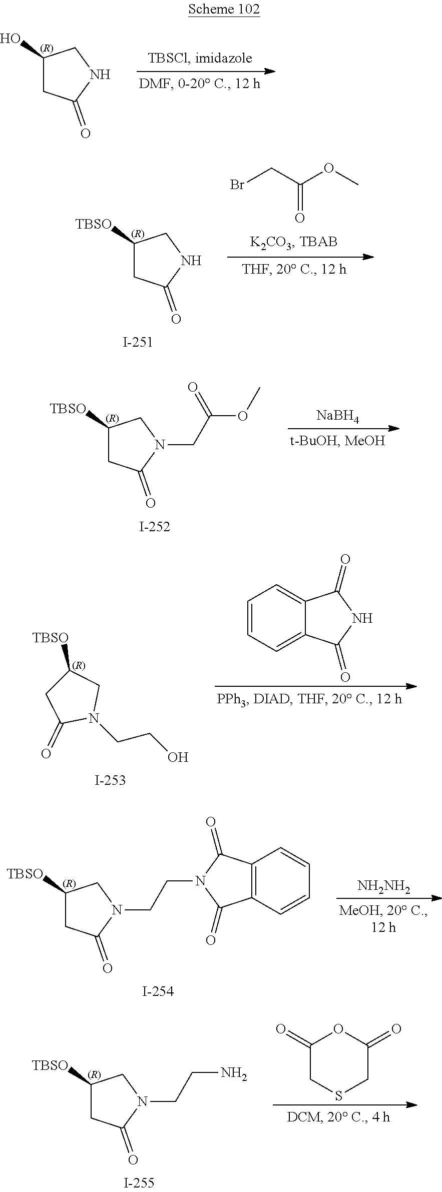

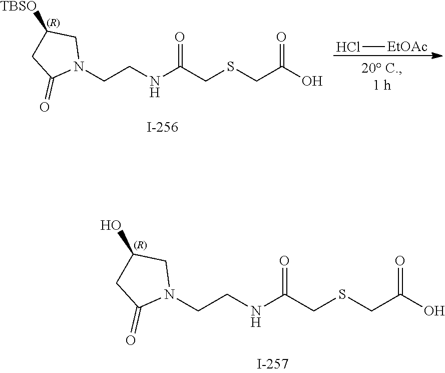

### Step 1: (R)-4-((tert-butyldimethylsilyl)oxy)pyrrolidin-2-one (I-251)

To a solution of (R)-4-hydroxypyrrolidin-2-one (cas: 22677-21-0, 2.02 g, 20 mmol) in DMF (10 ml) at 0-5° C. was added tert-butylchlorodimethylsilane (3.62 g, 24 mmol, 1.2 equiv.) and imidazole (3.40 g, 50 mmol, 2.5 equiv.). The reaction mixture was allowed to warm to ambient temperature and stirred for 12 hours. Water (50 mL) was then added, and the reaction mixture was stirred for 10 minutes. The white solid that precipitated was filtered and washed with water (10 mL) and dried under high vacuum to afford I-251 (4.05 g, 95% yield). MS (ESI, pos. ion) m/z: 216.1 (M+1).

### Step 2: methyl (R)-2-(4-((tert-butyldimethylsilyl)oxy)-2-oxopyrrolidin-1-yl)acetate (I-252)

To a solution of (R)-4-((tert-butyldimethylsilyl)oxy)pyrrolidin-2-one, I-251 (3.8 g 17.37 mmol) in THF (30 mL) was added methyl 2-bromoacetate (cas: 96-32-2, 4.03 g, 1.5 equiv.), potassium carbonate (0.84 g, 2.5 equiv.) and TBAB (4.94 g, 0.2 equiv.) under N₂. The reaction mixture was stirred at ambient temperature for 12 hours. The reaction was then quenched with water (10 mL), extracted with EtOAc (3×50 mL). The combined organic extracts were then washed with water (20 mL), brine (20 mL), dried (Na₂SO₄), and concentrated under reduced pressure to a residue, which was purified by silica gel chromatography to afford I-252 (0.71 g, 27% yield) as a pale oil. MS (ESI, pos. ion) m/z: 288.1 (M+1).

### Step 3: (R)-4-((tert-butyldimethylsilyl)oxy)-1-(2-hydroxyethyl)pyrrolidin-2-one (I-253)

To a solution of methyl (R)-2-(4-((tert-butyldimethylsilyl)oxy)-2-oxopyrrolidin-1-yl)acetate, I-252 (0.71 g, 2.47 mmol) in t-BuOH (10 mL) was added sodium borohydride (0.38 g, 4.0 equiv.) in MeOH (0.5 mL) under N₂. The reaction mixture was stirred for 2 hours at 80° C., then concentrated under reduced pressure to a residue. The residue was diluted with water (2 mL), and extracted with EtOAc (3×20 mL). The combined organic extracts were then washed with water (20 mL), brine (20 mL), dried (Na₂SO₄), and concentrated under reduced pressure to a residue, which was purified by silica gel chromatography to afford I-253 as a pale oil (0.43 g, 69% yield). MS (ESI, pos. ion) m/z: 260.2 (M+1).

### Step 4: (R)-2-(2-(4-((tert-butyldimethylsilyl)oxy)-2-oxopyrrolidin-1-yl)ethyl)isoindoline-1,3-dione (I-254)

To a solution of (R)-4-((tert-butyldimethylsilyl)oxy)-1-(2-hydroxyethyl)pyrrolidin-2-one, I-253 (0.43 g, 1.66 mmol) in THF (5 mL) at at 0-5° C. was added phthalimide (0.27 g, 1.1 equiv.) and PPh₃ (0.76 g, 1.5 equiv.). The reaction mixture was stirred for 0.5 hours under N₂, and DIAD (1.01 g, 3.0 equiv.) was added to the solution. The reaction mixture was allowed to warm to ambient temperature and stirred for 12 hours. The resulting mixture was quenched with water (5 mL) and extracted with DCM (3×20 mL). The combined organic extracts were washed with water (20 mL), brine (20 mL), dried (Na₂SO₄), and concentrated under reduced pressure to a residue, which was purified by silica gel chromatography to afford I-254 as a pale oil (0.48 g, 75% yield). MS (ESI, pos. ion) m/z: 389.2 (M+1).

### Step 5: (R)-1-(2-aminoethyl)-4-((tert-butyldimethylsilyl)oxy)pyrrolidin-2-one (I-255)

To a solution of (R)-2-(2-(4-((tert-butyldimethylsilyl)oxy)-2-oxopyrrolidin-1-yl)ethyl)isoindoline-1,3-dione, I-254 (0.48 g, 1.24 mmol) in MeOH (10 mL) at 0-5° C. was added NH₂NH₂ (85% in H₂O, 0.06 g, 1.2 equiv.). The reaction mixture was allowed to warm to ambient temperature and stirred for 12 hours and then filtered. The filtrate was concentrated under reduced pressure to afford the crude amine, I-255 (0.3 g) as a pale oil, which was used directly for next step. MS (ESI, pos. ion) m/z: 259.2 (M+1).

### Step 6: (R)-2-((2-((2-(4-((tert-butyldimethylsilyl)oxy)-2-oxopyrrolidin-1-yl)ethyl)amino)-2-oxoethyl)thio) acetic acid (I-256)

To a solution of (R)-1-(2-aminoethyl)-4-((tert-butyldimethylsilyl)oxy)pyrrolidin-2-one, I-255 (0.302 mg, 1.17 mmol) in DCM (10 mL) was added thiodiglycolic anhydride (0.20 mg, 1.52 mmol, 1.3 equiv.). The reaction mixture was stirred for 2 hours at 20° C. and then concentrated under reduced pressure to afford the crude product (0.5 g) as a pale oil. The crude product I-256 was used directly for the next step. MS (ESI, pos. ion) m/z: 391.2 (M+1).

### Step 6: (R)-2-((2-((2-(4-hydroxy-2-oxopyrrolidin-1-yl)ethyl)amino)-2-oxoethyl)thio) acetic acid (I-257)

To a solution of (R)-2-((2-((2-(4-((tert-butyldimethylsilyl)oxy)-2-oxopyrrolidin-1-yl)ethyl)amino)-2-oxoethyl)thio)acetic acid (0.50 g, 1.28 mmol) in DCM (5 mL) at 0-5° C. was added HCl in EtOAc (2M, 1.5 mL) under N₂. The reaction mixture was stirred for 1 hour. The reaction mixture was then concentrated under reduced pressure to afford the crude product as a pale oil. The crude product was purified by preparative HPLC (Mobile Phase: MeCN/H₂O with 0.1% TFA), Gradient: 2-8% MeCN) to give 33.3 mg (9% yield) of I-257 as a pale oil. MS (ESI, pos. ion) m/z: 277.1 (M+1). ¹H NMR (400 MHz, CDCl₃): 7.46 (s, 1H), 4.49-4.45 (m, 1H), 4.01 (ddd, 1H), 3.80 (ddd, 1H), 3.70 (dt, 2H), 3.39 (s, 2H), 3.35-3.28 (m, 1H), 3.20 (q, 2H), 3.08 (d, 1H), 2.81-2.74 (m, 1H), 2.57 (s, 1H).

### The Synthesis of I-264 Involved 7 Steps as Depicted in the Following Scheme 103

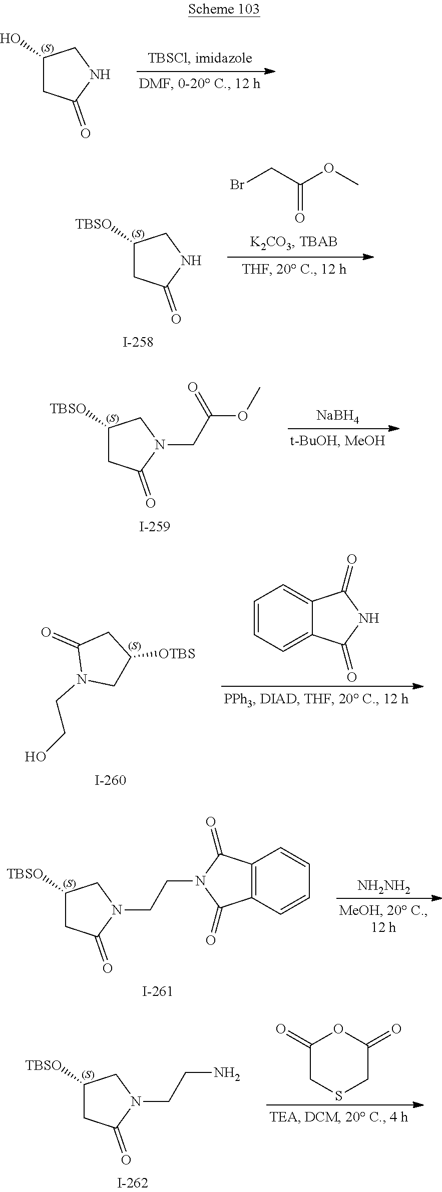

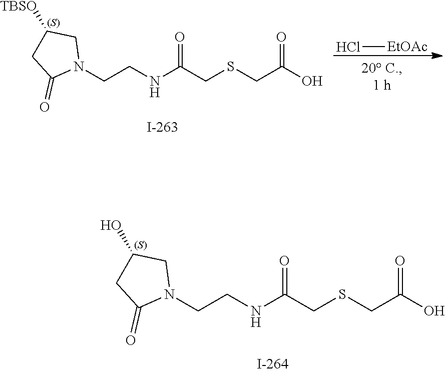

### Step 1: (S)-4-((tert-butyldimethylsilyl)oxy)pyrrolidin-2-one (I-258)

To a solution of (S)-4-hydroxypyrrolidin-2-one (cas: 68108-18-9, 5.0 g, 49.5 mmol) in DMF (20 mL) at 0-5° C. was added tert-butylchlorodimethylsilane (8.9 g, 1.2 equiv.) and imidazole (8.4 g, 2.5 equiv.). The reaction mixture was allowed to warm to ambient temperature and stirred for 12 hours. Water (50 mL) was then added and the reaction mixture was stirred for 10 minutes. The white precipitate that was formed was filtered and washed with water (15 mL) and then dried under high vacuum to afford I-258 (9.8 g, 92% yield). MS (ESI, pos. ion) m/z: 216.1 (M+1).

### Step 2: methyl (S)-2-(4-((tert-butyldimethylsilyl)oxy)-2-oxopyrrolidin-1-yl)acetate (I-259)

To a solution of (S)-4-((tert-butyldimethylsilyl)oxy)pyrrolidin-2-one, I-258 (4.9 g 22.79 mmol) in THF (40 mL) was added methyl 2-bromoacetate (5.2 g, 1.5 equiv.), potassium carbonate (7.9 g, 2.5 equiv.) and TBAB (1.47 g, 0.2 equiv.) under N₂. The reaction mixture was stirred for 12 hours at 20° C., quenched with water (10 mL), and extracted with EtOAc (3×50 mL). The combined organic extracts were washed with water (20 mL), brine (20 mL), dried (Na₂SO₄), and concentrated under reduced pressure to afford a residue, which was purified by silica gel chromatography to afford I-259 as a pale oil (1.1 g, 29% yield). MS (ESI, pos. ion) m/z: 288.1 (M+1).

### Step 3: (S)-4-((tert-butyldimethylsilyl)oxy)-1-(2-hydroxyethyl)pyrrolidin-2-one (I-260)

To a solution of methyl(S)-2-(4-((tert-butyldimethylsilyl)oxy)-2-oxopyrrolidin-1-yl)acetate, I-259 (1.1 g, 3.8 mmol) in t-BuOH (15 mL) was added sodium borohydride (0.58 g, 4.0 equiv.) in MeOH (1 mL) under N₂. The reaction mixture was stirred for 2 hours at 80° C. and then concentrated under reduced pressure to afford a residue. The residue was diluted with water (2 mL) and extracted with EtOAc (3×50 mL). The combined organic extracts were washed with water (20 mL), brine (20 mL), dried (Na₂SO₄), and concentrated under reduced pressure to afford a residue, which was purified by silica gel chromatography to afford I-260 as a pale oil (0.82 g, 83% yield). MS (ESI, pos. ion) m/z: 260.2 (M+1).

### Step 4: (S)-2-(2-(4-((tert-butyldimethylsilyl)oxy)-2-oxopyrrolidin-1-yl)ethyl)isoindoline-1,3-dione (I-261)

To a solution of (S)-4-((tert-butyldimethylsilyl)oxy)-1-(2-hydroxyethyl)pyrrolidin-2-one, I-260 (0.82 g, 3.17 mmol) in THF (10 mL) at 0-5° C. was added phthalimide (0.51 g, 1.1 equiv.) and PPh₃ (1.45 g, 1.5 equiv.). The reaction mixture was stirred for 0.5 hours under N₂ and DIAD (1.92 g, 3.0 equiv.) was added to the solution. The reaction mixture was allowed to warm to ambient temperature and stirred for 12 hours. The reaction was quenched with water (5 mL) and extracted with DCM (3×50 mL). The combined organic extracts were washed with water (20 mL), brine (20 mL), dried (Na₂SO₄) and concentrated under reduced pressure to afford a residue, which was purified by silica gel chromatography to afford I-261 as a pale oil (1.15 g, 94% yield). MS (ESI, pos. ion) m/z: 389.2 (M+1).

### Step 5: (S)-1-(2-aminoethyl)-4-((tert-butyldimethylsilyl)oxy)pyrrolidin-2-one (I-262)

To a solution of (S)-2-(2-(4-((tert-butyldimethylsilyl)oxy)-2-oxopyrrolidin-1-yl)ethyl)isoindoline-1,3-dione, I-261 (1.05 g, 1.24 mmol) in MeOH (10 mL) at 0-5° C. was added NH₂NH₂ (85% in H₂O, 0.12 g, 1.2 equiv.). The reaction mixture was allowed to warm to ambient temperature, stirred for 12 hours, and then filtered. The filtrate was concentrated under reduced pressure to afford the crude amine, I-262 (0.71 g) as a pale oil, which was used directly for the next step. MS (ESI, pos. ion) m/z: 259.2 (M+1).

### Step 6: (S)-2-((2-((2-(4-((tert-butyldimethylsilyl)oxy)-2-oxopyrrolidin-1-yl)ethyl)amino)-2-oxoethyl)thio) acetic acid (I-263)

To a solution of (S)-1-(2-aminoethyl)-4-((tert-butyldimethylsilyl)oxy)pyrrolidin-2-one (0.61 mg, 2.36 mmol) in DCM (10 mL) was added thiodiglycolic anhydride (0.41 mg, 1.3 equiv.) and TEA (0.05 g, 0.2 equiv.). The reaction mixture was stirred for 2 hours at 20° C. and was then concentrated under reduced pressure to give the crude product, I-263 (0.89 g) as a pale oil. The crude product was used directly for next step. MS (ESI, pos. ion) m/z: 391.2 (M+1).

### Step 6: (S)-2-((2-((2-(4-hydroxy-2-oxopyrrolidin-1-yl)ethyl)amino)-2-oxoethyl)thio) acetic acid (I-264)

To a solution of (S)-2-((2-((2-(4-((tert-butyldimethylsilyl)oxy)-2-oxopyrrolidin-1-yl)ethyl)amino)-2-oxoethyl)thio)acetic acid, I-263 (0.89 g, ca. 2.97 mmol) in DCM (10 mL) at 0-5° C. was added HCl in EtOAc (2M, 2.5 mL) under N₂. The reaction mixture was stirred for 1 hour and then concentrated under reduced pressure to afford the crude product as a pale oil. The crude product was purified by preparative HPLC (Mobile Phase: MeCN/H₂O (0.1% TFA), Gradient: 2-8% MeCN) to give 90 mg (11% yield) of I-264 as a pale oil. MS (ESI, pos. ion) m/z: 277.1 (M+1). ¹H NMR (400 MHz, CDCl₃): 7.40 (s, 1H), 4.44 (dd, 1H), 3.99 (dd, 1H), 3.76 (s, 1H), 3.69 (dt, 2H), 3.37 (s, 2H), 3.33-3.26 (m, 1H), 3.19 (q, 2H), 3.06 (d, J=14.4 Hz, 1H), 2.75 (ddd, 1H), 2.52 (d, 1H).

### The Synthesis of Compound I-271 Involved 7 Steps as Depicted in the Following Scheme 104

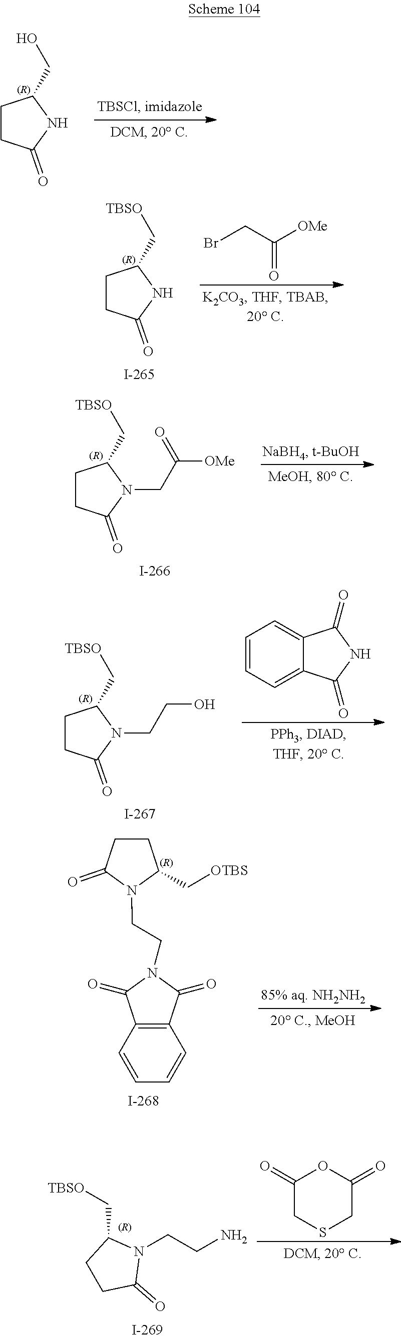

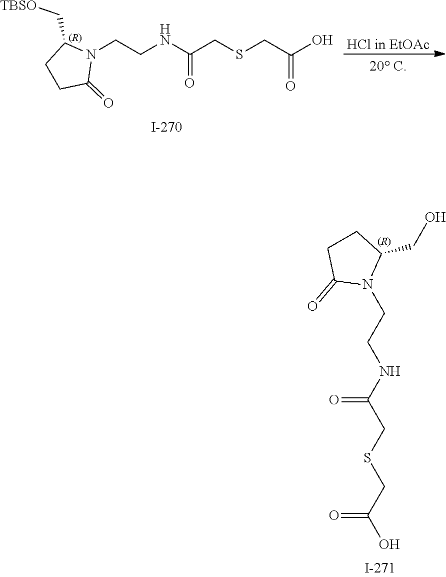

The chemical structure of the target compound was confirmed by 1HNMR and LC-MS with the purity >95% (HPLC 214 & 254 nm).

### (R)-2-((2-((2-(2-(hydroxymethyl)-5-oxopyrrolidin-1-yl)ethyl)amino)-2-oxoethyl)thio) acetic acid (I-271)

### Step 1. (R)-5-(((tert-butyldimethylsilyl)oxy)methyl)pyrrolidin-2-one (I-265)

To a solution of (R)-5-(hydroxymethyl)pyrrolidin-2-one (cas: 66673-40-3, 2 g, 17.4 mmol) in DCM (15 mL) was added imidazole (2.36 g, 2.0 equiv.) and TBSCl (3.14 g, 1.2 equiv.). The reaction mixture was stirred for 7 hours and was then quenched with water (20 mL). The aqueous phase was extracted with EtOAc (3×20 mL). The combined organic extracts were washed with water, brine, dried (Na₂SO₄), and concentrated under reduced pressure to afford a residue, which was purified by silica gel chromatography to provide silyl ether I-265 (2.9 g, 73% yield) as a pale yellow oil. MS (ESI, pos. ion) m/z: 230 (M+1).

### Step 2. methyl (R)-2-(2-(((tert-butyldimethylsilyl)oxy)methyl)-5-oxopyrrolidin-1-yl)acetate (I-266)

To a solution of (R)-5-(((tert-butyldimethylsilyl)oxy)methyl)pyrrolidin-2-one, I-265 (0.4 g, 1.75 mmol) in THF (10 mL) were added methyl 2-bromoacetate (0.4 g, 1.5 equiv.), K₂CO₃ (0.605 g, 2.5 equiv.), and TBAB (0.112 g, 2.0 equiv.). The reaction mixture was then stirred at ambient temperature for 10 hours and then quenched with water (20 mL). The aqueous phase was extracted with EtOAc (3×20 mL). The combined organic extracts were washed with water, brine, dried (Na₂SO₄), and concentrated under reduced pressure to a residue, which was purified by silica gel chromatography to provide ester I-266 (100 mg, 19%) as a pale yellow oil. MS (ESI, pos. ion) m/z: 302 (M+1).

### Step 3. (R)-5-(((tert-butyldimethylsilyl)oxy)methyl)-1-(2-hydroxyethyl)pyrrolidin-2-one (I-267)

To a solution of methyl (R)-2-(2-(((tert-butyldimethylsilyl)oxy)methyl)-5-oxopyrrolidin-1-yl)acetate, I-266 (0.2 g, 0.66 mmol) in t-BuOH (5 mL) and MeOH (0.2 mL) was added NaBH₄ (0.1 g, 4.0 equiv.) in one portion. The reaction mixture was heated to 80° C. for 2 hours and then cooled to ambient temperature and quenched with water (20 mL). The aqueous phase was then extracted with EtOAc (3×20 mL). The combined organic extracts were washed with water, brine, dried (Na₂SO₄) and concentrated under reduced pressure to a residue (220 mg), which was used directly for next step. MS (ESI, pos. ion) m/z: 274 (M+1).

### Step 4. (R)-2-(2-(2-(((tert-butyldimethylsilyl)oxy)methyl)-5-oxopyrrolidin-1-yl)ethyl)isoindoline-1,3-dione (I-268)

To a solution of (R)-5-(((tert-butyldimethylsilyl)oxy)methyl)-1-(2-hydroxyethyl) pyrrolidin-2-one, I-267 (0.22 g, 0.81 mmol), phthalimide (0.13 g, 1.1 equiv.) and PPh₃ (0.318 g, 1.5 equiv.) in anhydrous THF (5 mL) at 0-5° C. was added DIAD (0.49 g, 3.0 equiv.) under N₂. The reaction mixture was allowed to warm to ambient temperature and stirred for 7 hours and then concentrated under reduced pressure to a residue, which was and purified by preparative HPLC to afford the imide I-268 (145 mg, 28% yield). MS (ESI, pos. ion) m/z: 403 (M+1).

### Step 5. (R)-1-(2-aminoethyl)-5-(((tert-butyldimethylsilyl)oxy)methyl)pyrrolidin-2-one (I-269)

To a solution of (R)-2-(2-(2-(((tert-butyldimethylsilyl)oxy)methyl)-5-oxopyrrolidin-1-yl)ethyl)isoindoline-1,3-dione, I-268 (0.345 g, 0.858 mmol) in MeOH (10 mL) was added hydrazine hydrate (85% in H₂O, 0.05 g, 1.5 equiv.). The reaction mixture was stirred for 12 hours at ambient temperature and then filtered. The filtrate was concentrated under reduced pressure to give the crude product, I-269 (307 mg), which was used directly in next step. MS (ESI, pos. ion) m/z: 273 (M+1).

### Step 6. (R)-2-((2-((2-(2-(((tert-butyldimethylsilyl)oxy)methyl)-5-oxopyrrolidin-1-yl)ethyl)amino)-2-oxoethyl)thio) acetic acid (I-270)

To a solution of (R)-1-(2-aminoethyl)-5-(((tert-butyldimethylsilyl)oxy)methyl)pyrrolidin-2-one, I-269 (0.307 g, ca. 1.13 mmol) in DCM (5 mL) was added thiodiglycolic anhydride (178 mg, 1.2 equiv.) and TEA (22.8 mg, 0.2 equiv.). The reaction mixture was stirred for 2 hours and then concentrated in vacuo to give crude acid I-270 as a pale oil (0.5 g). The crude product was used directly for next step. MS (ESI, pos. ion) m/z: 405 (M+1).

### Step 7. (R)-2-((2-((2-(2-(hydroxymethyl)-5-oxopyrrolidin-1-yl)ethyl)amino)-2-oxoethyl)thio)acetic acid (I-271)

To a solution of (R)-2-((2-((2-(2-(((tert-butyldimethylsilyl)oxy)methyl)-5-oxopyrrolidin-1-yl)ethyl)amino)-2-oxoethyl)thio)acetic acid, I-270 (0.5 g, ca. 1.23 mmol) was added HCl in EtOAc (2M, 5 mL) and the reaction mixture was stirred for 2 hours and then concentrated in vacuo to afford the crude product. The crude product was purified by preparative HPLC (Mobile Phase: MeCN/H₂O (0.1% TFA)) to afford I-271 (0.1 g, 28% yield). MS (ESI, pos. ion) m/z: 291 (M+1). ¹H NMR (400 MHz, DMSO) δ 12.64 (s, 1H), 8.08 (m, 1H), 4.84 (s, 1H), 3.66 (m, 4.0 Hz, 1H), 3.56-3.47 (m, 2H), 3.41 (s, 3H), 3.28-3.09 (m, 4H), 3.02 m, 1H), 2.22 (m, 1H), 2.14 (m, 1H), 1.96 (m, 1H), 1.76 (m, 1H).

### The Synthesis of Compound I-278 Involved 7 Steps as Depicted in the Following Scheme 105

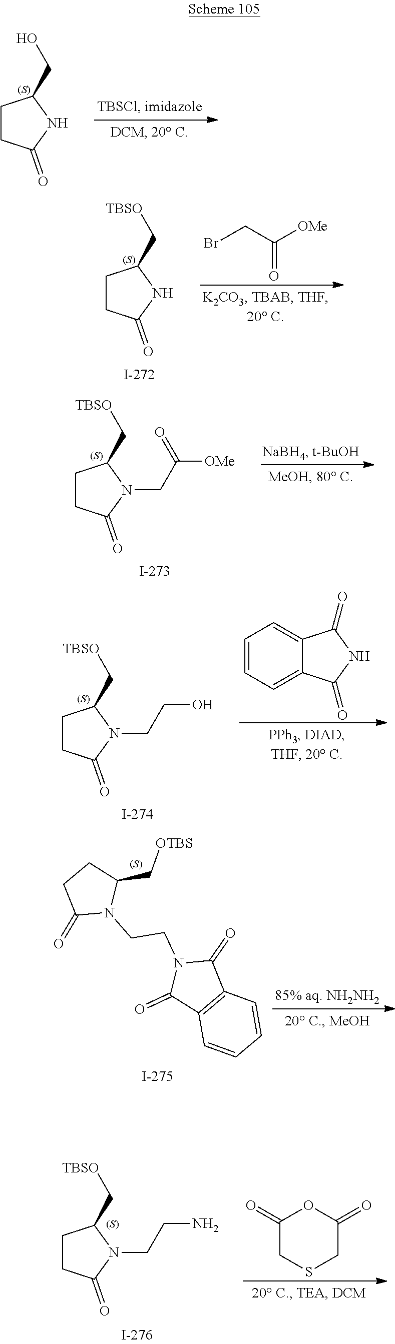

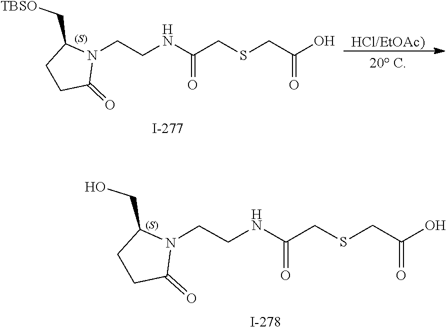

The chemical structure of the target compound was confirmed by ¹H NMR and LC-MS with the purity >95% (HPLC 214 & 254 nm).

### (S)-2-((2-((2-(2-(hydroxymethyl)-5-oxopyrrolidin-1-yl)ethyl)amino)-2-oxoethyl)thio)acetic acid (I-278)

The synthetic route for I-278 was similar to that for the synthesis of I-271 and commenced with (S)-5-(hydroxymethyl)pyrrolidin-2-one (cas: 17342-08-4). (S)-2-((2-((2-(2-(hydroxymethyl)-5-oxopyrrolidin-1-yl)ethyl)amino)-2-oxoethyl)thio)acetic acid, I-278 (pale oil, 120 mg, 15% yield) was isolated by preparative HPLC (Mobile Phase: MeCN/H₂O with 0.1% TFA). MS (ESI, pos. ion) m/z: 291 (M+1). ¹H NMR (400 MHz, DMSO) § 12.59 (s, 1H), 8.08 (m, 1H), 4.80 (s, 1H), 3.66 (m, 1H), 3.56-3.48 (m, 2H), 3.40 (m, 3H), 3.28-3.10 (m, 4H), 3.06-2.96 (m, 1H), 2.28-2.17 (m, 1H), 2.17-2.06 (m, 1H), 1.96 (m, 1H), 1.80-1.70 (m, 1H).

### Synthesis of I-279 is Depicted in Scheme 106

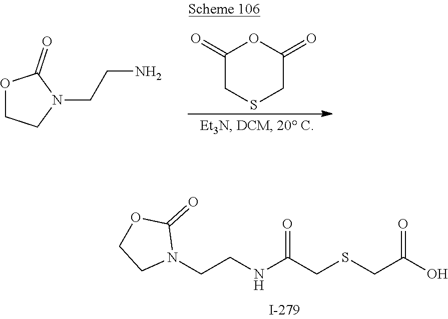 [SMILES: NCCN1CCOC1=O ; O=C1CSCC(=O)O1 ; O=C(O)CSCC(=O)NCCN1CCOC1=O]

### 2-((2-oxo-2-((2-(2-oxooxazolidin-3-yl)ethyl)amino)ethyl)thio)acetic acid (I-279)

To a solution of 3-(2-aminoethyl) oxazolidin-2-one (cas: 141778-93-0, 300 mg, 1.8 mmol) in DCM (20 mL) was added thiodiglycolic anhydride (360 mg, 1.5 equiv.) and TEA (90 mg, 0.5 equiv.). The reaction mixture was stirred at ambient temperature for 1 hour and then concentrated in vacuo. The residue obtained was purified by preparative HPLC (Mobile Phase: MeCN/H₂O (0.1% TFA), Gradient: 1-3% MeCN) to afford 87 mg (27% yield) of I-279 as a white solid. MS (ESI, pos. ion) m/z: 263.1 (M+1). ¹H NMR (400 MHz, DMSO) § 12.58 (s, 1H), 8.15 (m, 1H), 4.26-4.20 (m, 2H), 3.59-3.52 (m, 2H), 3.35 (s, 2H), 3.26-3.17 (m, 6H).

### The Synthesis of Compound I-282 Involved 3 Steps as Depicted in the Following Scheme 107

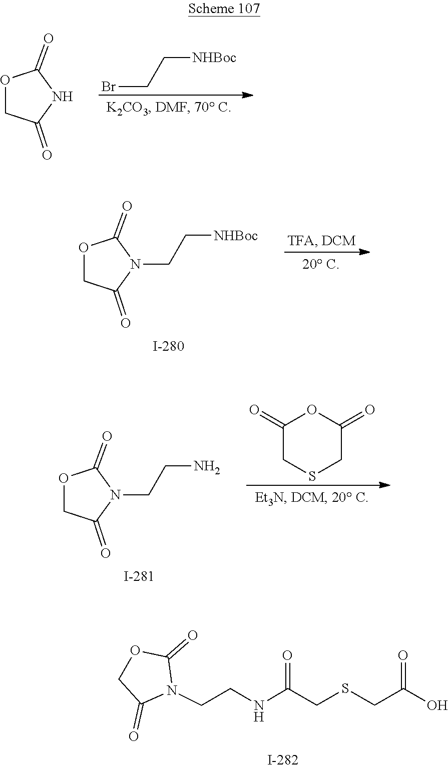

The chemical structure of the target compound was confirmed by 1HNMR and LC-MS with the purity >95% (HPLC 214 & 254 nm).

### 2-((2-((2-(2,4-dioxooxazolidin-3-yl)ethyl)amino)-2-oxoethyl)thio) acetic acid (I-282)

Step 1: Tert-butyl (2-(2,4-dioxooxazolidin-3-yl)ethyl)carbamate (I-280)

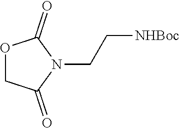 [SMILES: CC(C)(C)OC(=O)NCCN1C(=O)COC1=O]

To solution of oxazolidine-2,4-dione (cas: 2346-26-1, 0.1 g, 0.99 mmol) in DMF (5 mL) was added tert-butyl (2-bromoethyl)carbamate (cas: 39684-80-5, 0.265 g, 1.2 equiv.) and K₂CO₃ (0.274 g, 2.0 equiv.). The reaction mixture was heated to 70° C. for 2 hours. The reaction mixture was then cooled to ambient temperature, and quenched with water (20 mL). The aqueous phase was extracted with EtOAc (3×20 mL). The combined organic extracts were washed with water, brine, dried (Na₂SO₄), and concentrated under reduced pressure to afford a residue, which was purified by preparative TLC (Petroleum:EtOAc=1:1) to provide carbamate I-280 as a pale yellow oil (0.1 g, 41% yield). MS (ESI, pos. ion) m/z: 267 (M+Na).

### Step 2: 3-(2-aminoethyl)oxazolidine-2,4-dione (I-281)

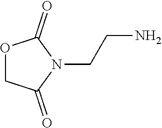

To a solution of tert-butyl (2-(2,4-dioxooxazolidin-3-yl)ethyl)carbamate, I-280 (0.1 g, 0.41 mmol) in DCM (2 ml) that had been pre-cooled to 0-5° C. was added a solution of TFA/DCM (1:1 v/v, 2 mL). The reaction mixture was then stirred at ambient temperature for 2 hours. When LC-MS showed that the reaction went to completion, the reaction solution was concentrated under reduced pressure to give the crude product, I-281 (pale oil, 176 mg). The crude product was used directly for next step. MS (ESI, pos. ion) m/z: 145 (M+1).

### Step 3: 2-((2-((2-(2,4-dioxooxazolidin-3-yl)ethyl)amino)-2-oxoethyl)thio) acetic acid (I-282)

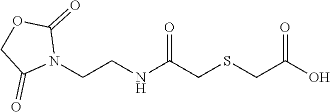 [SMILES: O=C(O)CSCC(=O)NCCN1C(=O)COC1=O]

To a solution of 3-(2-aminoethyl)oxazolidine-2,4-dione, I-281 (0.17 g, 1.17 mmol) and thiodiglycolic anhydride (0.087 g, 1.6 equiv. based on carbamate I-280) in DCM (5 mL) was added TEA (80 mg, 0.792 mmol). After 4 hours, the reaction mixture was concentrated to give the crude product. The crude product was purified by preparative HPLC (Mobile Phase: MeCN/H₂O with 0.1% TFA) to afford I-282 as a pale oil (50 mg, 2% yield). MS (ESI, pos. ion) m/z: 277.1 (M+1). ¹H NMR (400 MHz, DMSO): δ 8.21 (m, 1H), 4.77 (s, 2H), 3.47 (m, 2H), 3.33 (s, 2H), 3.28 (m, 2H), 3.16 (s, 2H).

### The Synthesis of Compound I-289 Involved 7 Steps as Depicted in the Following Scheme 108

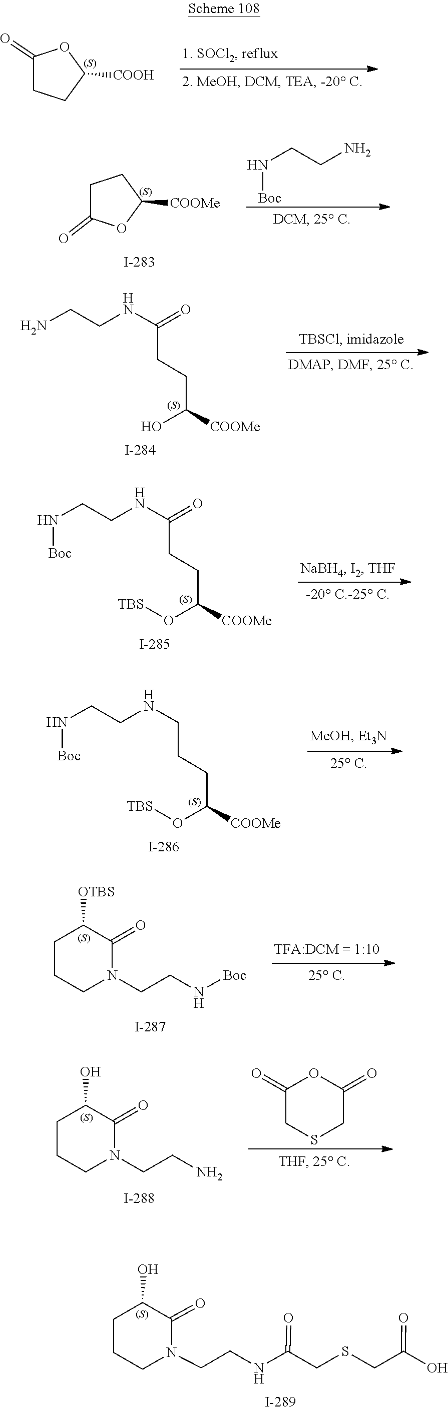

The chemical structure of the target compound was confirmed by 1HNMR and LC-MS with the purity >95% (HPLC 214 & 254 nm).

### Step 1: Methyl-(S)-5-Oxotetrahydrofuran-2-Carboxylate (I-283)

To a solution of (S)-5-oxotetrahydrofuran-2-carboxylic acid (cas: 21461-84-7, 7 g 53.8 mmol) was added SOCl₂ (17.95 g, 2.8 equiv., 11 mL) and the reaction mixture was refluxed at 80° C. for 4 hours under N₂. The reaction mixture was then allowed to cool to ambient temperature and stirred for 12 hours. The excess SOCl₂ was removed under reduced pressure. The residue was diluted with DCM (70 mL); a solution of MeOH (2.1 mL, 53.8 mmoL) and TEA (6.56 g, 64.5 mmoL) was added at −20° C. The reaction mixture was warmed to ambient temperature and stirred for 12 hours. The reaction was then quenched with H₂O (40 mL) and extracted with EtOAc (20 mL×3). The combined organic extracts were dried over MgSO₄ and concentrated under reduced pressure to give the crude product. The crude product was purified by silica gel chromatography (Petroleum ether:EtOAc=1:1) to afford I-283 as a pale oil (4.5 g, 58% yield). MS (ESI, pos. ion) m/z: 236 (M+1).

### Step 2: Methyl-(S)-5-((2-aminoethyl)amino)-2-hydroxy-5-oxopentanoate (I-284)

To a solution of methyl (S)-5-oxotetrahydrofuran-2-carboxylate, I-283 (3 g, 20.83 mmol) in DCM (20 mL) was added tert-butyl (2-aminoethyl)carbamate (cas: 57260-73-8, 230 mg). The reaction mixture was then stirred at ambient temperature for 48 hours. When the LC-MS showed the reaction went to completion, the reaction mixture was concentrated under reduced pressure to give the crude product, I-284. The crude product (3.5 g, 83% yield) was used directly in next step. MS (ESI, pos. ion) m/z: 205 (M+1).

### Step 3: Methyl-(S)-5-((2-((tert-butoxycarbonyl)amino)ethyl)amino)-2-((tert-butyldimethylsily)oxy)-5-oxopentanoate (I-285)

To a solution of methyl(S)-5-((2-aminoethyl)amino)-2-hydroxy-5-oxopentanoate, I-284 (3.5 g, 13.98 mmol) in anhydrous DMF (30 mL) was added imidazole (2.38 g, 2.5 equiv.) and TBSCl (3.79 g, 1.8 equiv.). The reaction mixture was stirred at ambient temperature for 3 hours. The reaction was quenched with H₂O (20 mL) and extracted with EtOAc (20 mL×3). The combined organic extracts were concentrated to afford the crude product. The crude product was purified by silica gel chromatography (Petroleum ether:EtOAc=1:3) to provide I-285 as a pale oil (3.6 g, 49% yield). MS (ESI, pos. ion) m/z: 419 (M+1).

### Step 4: Methyl(S)-8-((tert-butoxycarbonyl)amino)-2-((tert-butyldimethylsilyl)oxy)octanoate (I-286)

A solution of methyl (S)-5-((2-((tert-butoxycarbonyl)amino)ethyl)amino)-2-((tert-butyldimethylsilyl)oxy)-5-oxopentanoate, I-285 (2 g, 5.48 mmol) and NaBH₄ (0.373 g, 1.8 equiv.) in THF (anhydrous, 26 mL) was added to a solution of iodine (1.25 g, 0.9 equiv.) in THF (anhydrous, 10 mL) at −20° C. under N₂. The reaction mixture was gradually warmed to ambient temperature over 8 hours. When the LC-MS showed that the reaction went to completion, the reaction was quenched with H₂O (20 mL) and extracted with EtOAc (30 mL×3). The combined organic extracts were dried over MgSO₄ and concentrated to give the crude product. The crude product, I-286 (1.8 g, 93% yield) was used directly in next step. MS (ESI, pos. ion) m/z: 404 (M+1).

### Step 5: Tert-butyl(S)-(2-(3-((tert-butyldimethylsilyl)oxy)-2-oxopiperidin-1-yl)ethyl)carbamate (I-287)

To a solution of methyl(S)-8-((tert-butoxycarbonyl)amino)-2-((tert-butyldimethylsilyl)oxy) octanoate, I-286 (3.6 g, 8.9 mmol) in DCM (30 mL) was added TEA (0.27 g, 0.3 mmol). The reaction mixture was stirred for 48 hours and was concentrated under reduced pressure to give the crude product. The crude product was purified by silica gel chromatography (Petroleum ether:EtOAc=3:1) to provide lactam I-287 as a pale oil (1.2 g, 36% yield). MS (ESI, pos. ion) m/z: 373 (M+1), 395 (M+Na).

### Step 6: (S)-1-(2-aminoethyl)-3-hydroxypiperidin-2-one (I-288)

A solution of tert-butyl(S)-(2-(3-((tert-butyldimethylsilyl)oxy)-2-oxopiperidin-1-yl)ethyl)carbamate, I-287 (1.2 g, 3.22 mmol) was added to a solution of TFA/DCM (1 mL/3 mL) at 0° C. The reaction mixture was stirred for 2 hours and concentrated under reduced pressure to afford the crude product. The crude product, I-288 (0.8 g, pale oil) was used directly for next step. MS (ESI, pos. ion) m/z: 159 (M+1).

### Step 7: (S)-2-((2-((2-(3-hydroxy-2-oxopiperidin-1-yl)ethyl)amino)-2-oxoethyl)thio) acetic acid (I-289)

To a solution of (S)-1-(2-aminoethyl)-3-hydroxypiperidin-2-one, I-288 (0.89 g 5.6 mmol) and thiodiglycolic anhydride (1.11 g, ca. 1.5 equiv.) in 10 mL of DCM was added TEA (0.114 g, 0.2 equiv.). The reaction mixture was stirred for 2 hours and concentrated under reduced pressure to give the crude product. The crude product was purified by preparative HPLC (Mobile Phase: MeCN/H₂O (0.1% TFA), Gradient: 5-15% MeCN) to give I-289 (50 mg, 3% yield) as a pale oil. MS (ESI, pos. ion) m/z: 291.1 (M+1). ¹H NMR (400 MHz, DMSO) & 12.63 (s, 1H), 8.11 (m, 1H), 3.84 (m, 1H), 3.45-3.37 (m, 2H), 3.35 (s, 2H), 3.30-3.24 (m, 2H), 3.24-3.19 (m, 3H), 3.18 (s, 2H), 1.99-1.89 (m, 1H), 1.81 (m, 1H), 1.70 (m, 1H), 1.64-1.52 (m, 1H).

### The Synthesis of I-296 Involved 7 Steps as Depicted in the Following Scheme 109

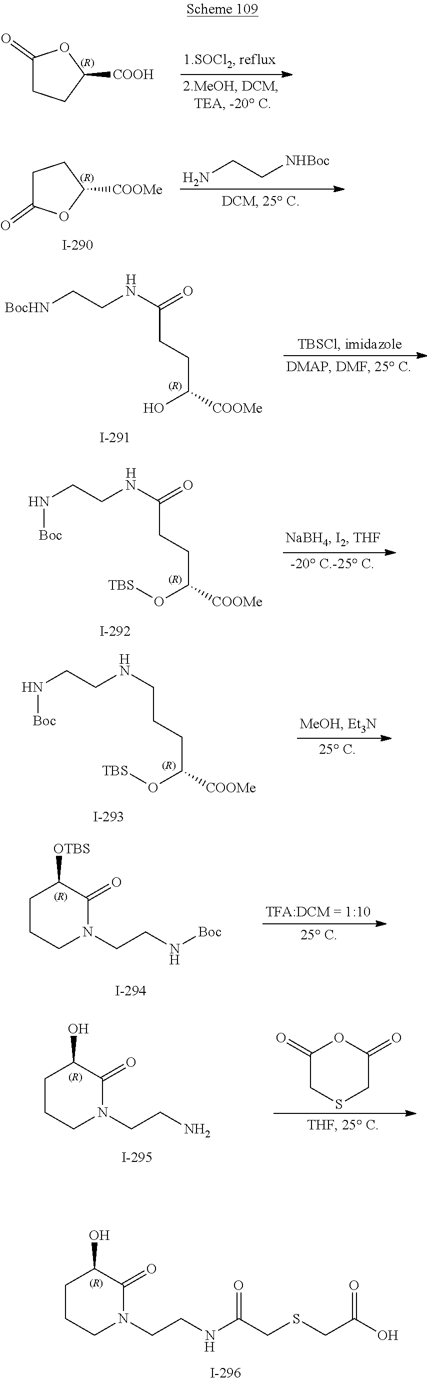

### (R)-2-((2-((2-(3-hydroxy-2-oxopiperidin-1-yl)ethyl)amino)-2-oxoethyl)thio) acetic acid (I-296)

The synthetic route for I-296 was similar to that of I-288 and commenced with (R)-5-oxotetrahydrofuran-2-carboxylic acid (cas: 53558-93-3). I-296 (pale oil, 39 mg, 3% yield) was isolated by preparative HPLC (Mobile Phase: MeCN/H₂O (0.1% TFA), Gradient: 5-15% MeCN). MS (ESI, pos. ion) m/z: 291.1 (M+1). ¹H NMR (400 MHz, DMSO) δ 12.59 (s, 1H), 8.10 (s, 1H), 5.00 (s, 1H), 3.84 (m, 1H), 3.42 (s, 2H), 3.28 (m, 3H), 3.21 (m, 3H), 3.18 (s, 2H), 1.95 (m, 1H), 1.87-1.76 (m, 1H), 1.75-1.65 (m, 1H), 1.58 (m, 1H).

### The Synthesis of I-301 Involved 5 Steps as Depicted in the Following Scheme 110

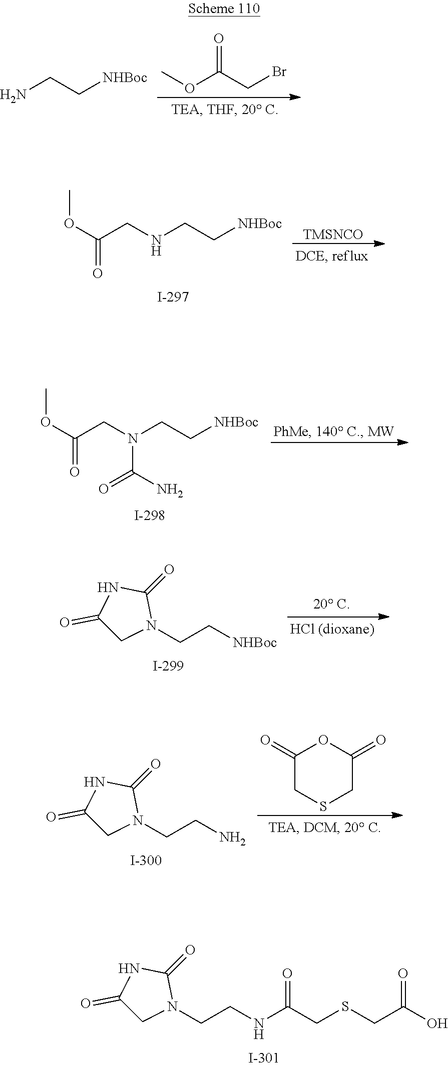

### Step 1: Methyl(2-((tert-butoxycarbonyl)amino)ethyl)glycinate (I-297)

A solution of tert-butyl (2-aminoethyl)carbamate (cas: 57260-73-8, 3.2 g, 20 mmol), methyl 2-bromoacetate (cas: 96-32-2, 3.0 g, 1 equiv.) and TEA (4 mL, 2.9 equiv.) in 100 mL of THF was stirred at ambient temperature for 4 hours. Upon completion of the reaction, the reaction mixture was filtered and the filtrate was concentrated and the crude product, I-297 (5.3 g) was used in next step directly. MS (ESI, pos. ion) m/z: 255.2 (M+23).

### Step 2: Methyl N-(2-((tert-butoxycarbonyl)amino)ethyl)-N-carbamoylglycinate (I-298)

A solution of methyl(2-((tert-butoxycarbonyl)amino)ethyl)glycinate, I-297 and trimethylsilyl isocyanate (cas: 1118 Feb. 1, 4.5 mL, 1.7 equiv.) in DCE (50 mL) was heated to 90° C. for 48 hours. LC-MS at this point indicated that the reaction was not complete. The reaction mixture was concentrated, and purified by silica gel chromatography to afford I-298 as a pale oil (4.2 g, 76% over 2 steps). MS (ESI, pos. ion) m/z: 298.2 (M+23).

### Step 3: Tert-butyl (2-(2,4-dioxoimidazolidin-1-yl)ethyl)carbamate (I-299)

Methyl N-(2-((tert-butoxycarbonyl)amino)ethyl)-N-carbamoylglycinate, I-298 (4.2 g, 15 mmol) in toluene (20 mL) was heated to 140° C. under Microwave irradiation for 30 minutes. The reaction mixture was then concentrated and purified by silica gel chromatography to afford I-299 as a pale oil (1.5 g, 41% yield). MS (ESI, pos. ion) m/z: 266.2 (M+23).

### Step 4: 1-(2-aminoethyl) imidazolidine-2,4-dione (I-300)

A solution of tert-butyl (2-(2,4-dioxoimidazolidin-1-yl)ethyl)carbamate, I-299 (1.2 g. 4.9 mmol) in HCl (4M in dioxane) (18 mL) was stirred at ambient temperature for 2 hours. Upon completion of the reaction, the reaction mixture was concentrated and the crude amine I-300 was used in the next step directly. MS (ESI, pos. ion) m/z: 144.2 (M+1).

### Step 5: 2-((2-((2-(2,4-dioxoimidazolidin-1-yl)ethyl)amino)-2-oxoethyl)thio) acetic acid (I-301)

A solution of 1-(2-aminoethyl) imidazolidine-2,4-dione, I-300 (crude, ca. 4.9 mmol) and thiodiglycolic anhydride (1.2 g, 1.8 equiv.) in 30 mL of DCM was stirred for 15 min. Upon completion of the reaction, the reaction mixture was concentrated in vacuo to give the crude product as a pale oil. Half the crude product was purified by preparative HPLC (Mobile Phase: MeCN/H₂O with 0.1% TFA), Gradient: 1-3% MeCN) to give 130 mg of I-301 as a white solid (19% yield). MS (ESI, pos. ion) m/z: 276.2 (M+1). ¹H NMR (400 MHz, DMSO) § 12.61 (s, 1H), 10.72 (s, 1H), 8.15 (t, 1H), 3.94 (s, 2H), 3.33 (s, 2H), 3.31-3.25 (m, 2H), 3.25-3.20 (m, 2H), 3.18 (s, 2H).

### The Synthesis of I-307 Involved 6 Steps as Depicted in the Following Scheme 111

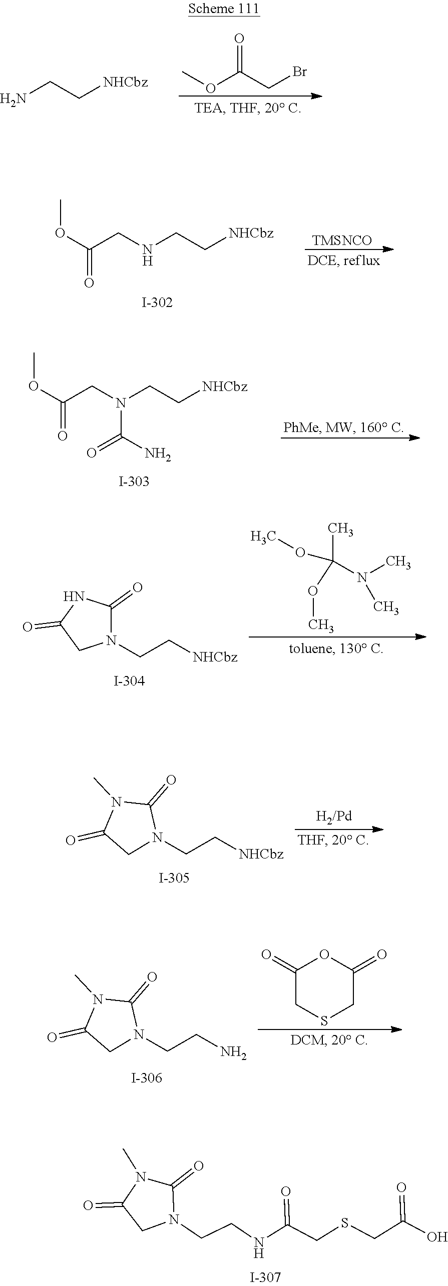

### 2-((2-((2-(3-methyl-2,4-dioxoimidazolidin-1-yl)ethyl)amino)-2-oxoethyl)thio)acetic acid (I-307)

Steps 1-3 were similar to those used in the preparation of carboxylic acid I-301.

### Step 4: Benzyl(2-(3-methyl-2,4-dioxoimidazolidin-1-yl)ethyl)carbamate (I-305)

To a solution of benzyl(2-(2,4-dioxoimidazolidin-1-yl)ethyl)carbamate, I-304 (600 mg, 2.17 mmol) in toluene (20 mL) was added N,N-dimethylacetamide dimethyl acetal (cas: 18871-66-4, 1.3 mL, 4.0 equiv.) in one portion at 25° C. The resulting mixture was heated to 130° C. for 2 hours. After cooling to ambient temperature, the reaction mixture was concentrated in vacuo. The residue was purified by preparative TLC to give 500 mg (79% yield) of carbamate I-305 as a pale yellow oil. MS (ESI, pos. ion) m/z: 292.1 (M+1).

### Step 5: Tert-butyl 1-(((benzyloxy)carbonyl)amino)cyclopropane-1-carboxylate (I-306)

To a solution of benzyl(2-(3-methyl-2,4-dioxoimidazolidin-1-yl)ethyl)carbamate, I-305 (500 mg, 1.72 mmol) in 20 mL of THF was added 50 mg of 10% Pd/C. The reaction was stirred at 25° C. for 10 hours under H₂. Upon completion of the reaction, the reaction mixture was filtered and the filtrate was concentrated in vacuo. The residue was used in next step directly without purification. MS (ESI, pos. ion) m/z: 158.1 (M+1).

### Step 6: 2-((2-((2-(3-methyl-2,4-dioxoimidazolidin-1-yl)ethyl)amino)-2-oxoethyl)thio)acetic acid (I-307)

To a solution of tert-butyl 1-(((benzyloxy)carbonyl)amino)cyclopropane-1-carboxylate, I-306 (500 mg, 3.18 mmol) in DCM (20 mL) was added thiodiglycolic anhydride (630 mg, 1.5 equiv.). The reaction mixture was then stirred at 25° C. for 1 hour. Upon completion of the reaction, the solvent was removed in vacuo, and the crude product was purified by preparative HPLC (Mobile Phase: MeCN/H₂O with 0.1% TFA), gradient: 1-3% MeCN) to give 250 mg (27% yield) of I-307 as a pale oil. MS (ESI, pos. ion) m/z: 290.1 (M+1). ¹H NMR (400 MHz, DMSO) δ 8.15 (s, 1H), 3.98 (s, 2H), 3.34 (d, 2H), 3.33 (s, 2H), 3.25 (t, 2H), 3.17 (s, 2H), 2.83 (s, 3H).

### The Synthesis of I-311 Involved 5 Steps as Depicted in the Following Scheme 112

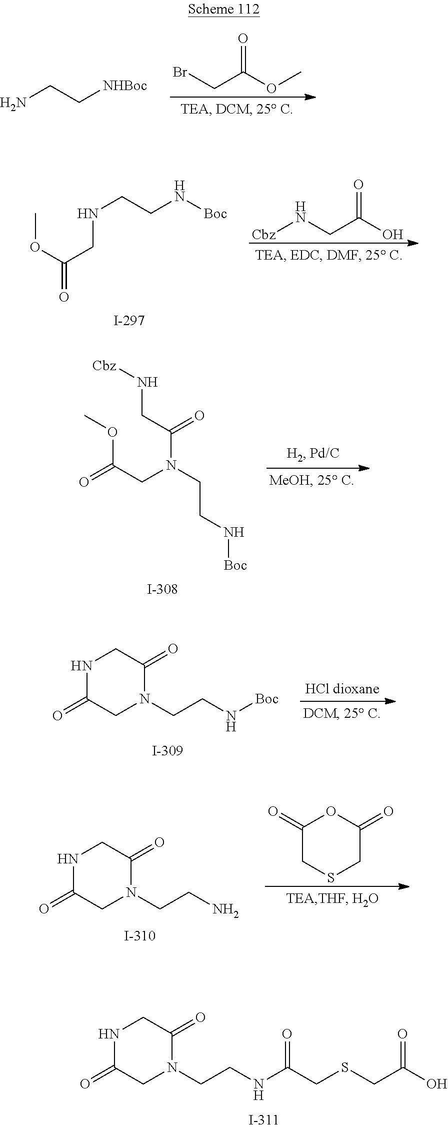

### 2-((2-((2-(2,5-dioxopiperazin-1-yl)ethyl)amino)-2-oxoethyl)thio)acetic acid (I-311)

### Step 2: Methyl N-(((benzyloxy)carbonyl)glycyl)-N-(2-((tert-butoxycarbonyl) amino) ethyl) glycinate (I-308)

To a solution of benzyloxycarbonylglycine (3.1 g, 15 mmol, cas: 1138-80-3) in DMF (30 mL) and methyl(2-((tert-butoxycarbonyl)amino)ethyl)glycinate, I-297 (1.8 g, 7.8 mmol) was added TEA (2.4 g, 24 mmol), EDC (4.6 g, 24 mmol), and then the reaction mixture was stirred at 25° C. for 15 hours. The reaction mixture was then diluted with EtOAc (100 mL) and was sequentially washed with aqueous 5% NaHCO₃ (100 mL×3), aqueous 5% citric acid solution (100 mL×3) and brine (100 mL×3). The EtOAc extracts were dried over anhydrous magnesium sulfate and purified by silica gel column chromatography (3% MeOH/DCM) to give 2.1 g of I-308 as a pale oil. Yield: 64%. MS (ESI, pos. ion) m/z: 446.1 (M+23).

### Step 3: Tert-butyl (2-(2,5-dioxopiperazin-1-yl)ethyl)carbamate (I-309)

To a solution of methyl N-(((benzyloxy)carbonyl)glycyl)-N-(2-((tert-butoxycarbonyl)amino)ethyl)glycinate, 1-308 (2.1 g, 5 mmol) in 30 mL of MeOH was added 500 mg of 10% Pd/C. The reaction mixture was stirred at ambient temperature for 10 hours under H₂ (1 atm). Upon completion of the reaction, Pd/C was filtered and the filtrate was concentrated in vacuo. The residue was purified by silica gel column chromatography (3% MeOH/DCM) to give 800 mg of I-309 as a pale oil. Yield: 64%. MS (ESI, pos. ion) m/z: 280.1 (M+23).

### Step 4: Benzyl(2-(3-methyl-2,4-dioxoimidazolidin-1-yl)ethyl)carbamate (I-310)

To a solution of tert-butyl (2-(2,5-dioxopiperazin-1-yl)ethyl)carbamate, I-309 (400 mg, 1.6 mmol) in 15 mL of DCM was added 15 mL of 4 M HCl in dioxane. The reaction mixture was stirred at 25° C. for 1 hour. Upon completion of the reaction, the mixture was concentrated under reduced pressure to give 360 mg of crude amine I-310 as a white solid, which was used in next step. MS (ESI, pos. ion) m/z: 158.1 (M+1).

### Step 5: 2-((2-((2-(2,5-dioxopiperazin-1-yl)ethyl)amino)-2-oxoethyl)thio)acetic acid (I-311)

To a solution of benzyl(2-(3-methyl-2,4-dioxoimidazolidin-1-yl)ethyl)carbamate (300 mg, 1.91 mmol) in THF/H₂O (10 mL/1 mL) was added TEA (200 mg, 2 mmol) and thiodiglycolic anhydride (504 mg, 3.82 mmol). The reaction mixture was stirred at 25° C. for 1 hour. Upon completion of the reaction, the solvent was removed under reduced pressure and the resulting residue was purified by preparative HPLC (Mobile Phase: MeCN/H₂O (with 0.1% formic acid) Gradient: 2-8% MeCN) to give 65 mg of carboxylic acid I-311 as a pale oil. Yield: 12%. MS (ESI, pos. ion) m/z: 290.1 (M+1). 1H NMR (400 MHz, DMSO) δ 8.12 (dd, J=12.1, 6.1 Hz, 2H), 3.92 (s, 2H), 3.76 (s, 1H), 3.38-3.31 (m, 4H), 3.23 (dd, J=11.8, 5.9 Hz, 1H).

### The Synthesis of I-320 Involved 9 Steps as Depicted in the Following Scheme 113

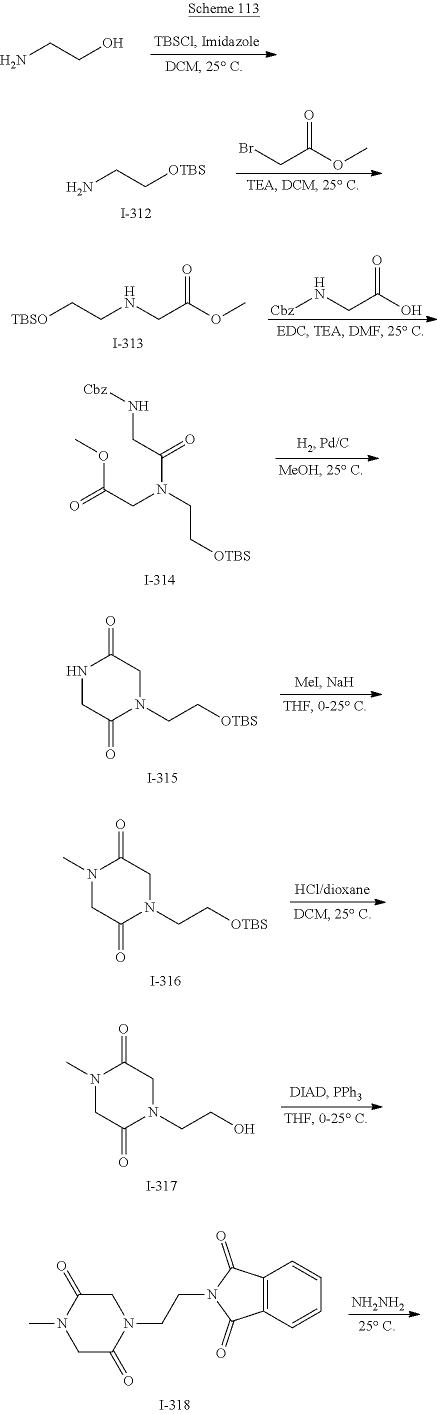

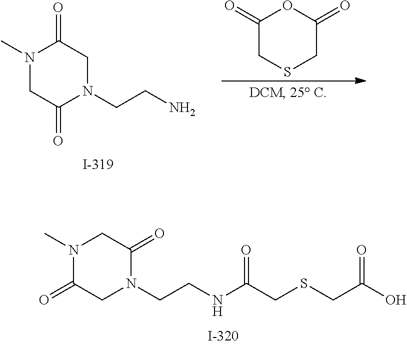 [SMILES: CN1CC(=O)N(CCN)CC1=O ; O=C1CSCC(=O)O1 ; CN1CC(=O)N(CCNC(=O)CSCC(=O)O)CC1=O]

### 2-((2-((2-(4-methyl-2,5-dioxopiperazin-1-yl)ethyl)amino)-2-oxoethyl)thio)acetic acid (I-320)

### Step 1: 2-((tert-butyldimethylsilyl)oxy)ethan-1-amine (I-312)

To a solution of 2-aminoethan-1-ol (1.2 g, 20 mmol), DMAP (220 mg, 1.8 mmol) and imidazole (2.7 g, 40 mmol) in 30 ml of DCM was added TBSCl (3.1 g, 21 mmol). The resulting mixture was stirred at ambient temperature for 16 hours. Upon completion of the reaction, 50 mL of water was added to the reaction mixture, which was then extracted with DCM (30 mL×3). The combined organic extracts were dried over Na₂SO₄, then concentrated in vacuo to give 3.1 g of I-312 as a pale oil. Yield: 89%. MS (ESI, pos. ion) m/z: 176.1 (M+1).

### Step 2: Methyl(2-((tert-butyldimethylsilyl)oxy)ethyl)glycinate (I-313)

To a solution of 2-((tert-butyldimethylsilyl)oxy)ethan-1-amine, I-312 (1.75 g, 10 mmol,) in 30 mL of DCM was added methyl 2-bromoacetate (1.6 g, 1.1 equiv., cas #: 96-32-2) and TEA (2.0 g, 2.0 equiv.). The reaction mixture was stirred at ambient temperature for 10 hours under N₂. Upon completion of the reaction, the solvent was removed in vacuo and the mixture was purified by column chromatography (5-10% MeOH in DCM) to give 2 g of I-313 as a yellow oil. Yield: 62%. MS (ESI, pos. ion) m/z: 248.1 (M+1).

### Step 3: Methyl N-(((benzyloxy)carbonyl)glycyl)-N-(2-((tert-butoxycarbonyl) amino) ethyl) glycinate (I-314)

To a solution of benzyloxycarbonylglycine (3.1 g, 15 mmol, cas #: 1138-80-3) and amine I-313 (1.8 g, 7.28 mmol) in 30 mL of DMF was added TEA (2.4 g, 24 mmol) and the reaction mixture was stirred at 0° C. To this reaction mixture was added EDC (3.5 g, 18 mmol) and stirring continued at 0° C. for 2 hours, after which the reaction mixture was allowed to warm to ambient temperature and stirred for 15 hours. The reaction mixture was diluted with EtOAc (100 mL) and sequentially washed with aqueous 5% sodium bicarbonate solution (100 mL×3), aqueous 5% citric acid solution (100 mL×3), aqueous saturated sodium chloride solution (100 mL×3). The EtOAc extracts were dried over Na₂SO₄ and purified by silica gel column chromatography (3.8% MeOH/DCM) to give 3 g of amide I-314 as a yellow oil. Yield: 43%. MS (ESI, pos. ion) m/z: 461.1 (M+23).

### Step 4:1-(2-((tert-butyldimethylsilyl)oxy)ethyl)piperazine-2,5-dione (I-315)

To a solution of methyl N-(((benzyloxy)carbonyl)glycyl)-N-(2-((tert-butoxycarbonyl)amino)ethyl)glycinate, I-314 (2.1 g, 4.8 mmol) in 30 mL of MeOH was added 500 mg of 10% Pd/C, The reaction mixture was stirred at ambient temperature for 10 hours under H₂ (1 atm). Upon completion of the reaction, Pd/C was filtered and the filtrate was concentrated under reduced pressure. The residue was purified by silica gel column chromatography (3% MeOH/DCM) to give 720 mg of diketopiperazine I-315 as a pale oil. Yield: 55%. MS (ESI, pos. ion) m/z: 273.1 (M+1).

### Step 5:1-(2-((tert-butyldimethylsilyl)oxy)ethyl)-4-methylpiperazine-2,5-dione (I-316)

To a solution of 1-(2-((tert-butyldimethylsilyl)oxy)ethyl)piperazine-2,5-dione, I-315 (720 mg, 2.64 mmol) and NaH (500 mg, 8.3 equiv., 60% dispersion in mineral oil) in 20 mL of THF at 0° C. was added MeI (1.1 g, 3.0 equiv.). The reaction mixture was stirred at ambient temperature for 1 hour. Upon completion of the reaction, the solvent was removed in vacuo, and the residue obtained was purified by column chromatography (DCM) to give 680 mg of diketopiperazine I-316 as a yellow oil, yield: 90%. MS (ESI, pos. ion) m/z: 287.1 (M+1).

### Step 6:1-(2-hydroxyethyl)-4-methylpiperazine-2,5-dione (1-317)

To a solution of 1-(2-((tert-butyldimethylsilyl)oxy)ethyl)-4-methylpiperazine-2,5-dione, I-316 (800 mg, 2.8 mmol) in DCM (8 mL) cooled in an ice-water bath was added HCl in Dioxane (30% v/v, 8 mL) under N₂. The reaction mixture was stirred for 0.5 hours. Upon completion of the reaction, the reaction mixture was concentrated under reduced pressure to give 608 mg of I-317 as a pale oil, which was used directly for next step. MS (ESI, pos. ion) m/z: 173.1 (M+1).

### Step 7:2-(2-(4-methyl-2,5-dioxopiperazin-1-yl)ethyl)isoindoline-1,3-dione (I-318)

To a solution of 1-(2-hydroxyethyl)-4-methylpiperazine-2,5-dione, 1-317 (600 mg, 3.8 mmol) in THF (20 mL) was added phthalimide (cas: 85-41-6, 592 mg, 1.05 equiv.) and PPh₃ (1.5 g, 1.5 equiv.). The reaction mixture was stirred for 0.5 h in ice bath under N₂. DIAD (2.3 g, 3.0 equiv.) was the added dropwise to the solution. The reaction mixture was allowed to warm to ambient temperature and stirred for 6 hours. Upon completion of the reaction, the reaction mixture was quenched with water (5 mL) and extracted with DCM (3×20 mL). The combined organic extracts were washed with water (20 mL), brine (20 mL), and dried over Na₂SO₄, then concentrated and the residue obtained was purified by column chromatography (50-100% EtOAc) to give 480 mg of I-318 as a yellow solid, yield: 42%. MS (ESI, pos. ion) m/z: 302.1 (M+1).

### Step 8: 1-(2-aminoethyl)-4-methylpiperazine-2,5-dione (I-319)

To a solution of 2-(2-(4-methyl-2,5-dioxopiperazin-1-yl)ethyl)isoindoline-1,3-dione, I-318 (300 mg, 1 mmol) in MeOH (5 mL) that had been cooled in an ice-water bath was added NH₂NH₂ (85% in H₂O, 300 mg, 5.5 equiv.). The reaction mixture was allowed to warm to ambient temperature and stirred for 12 hours. Upon completion of the reaction, the slurry was filtered and the filtrate was concentrated to afford crude amine I-319, which was used directly for the next step (pale oil, 280 mg). MS (ESI, pos. ion) m/z: 172.1 (M+1).

### Step 9: 2-((2-((2-(4-methyl-2,5-dioxopiperazin-1-yl)ethyl)amino)-2-oxoethyl)thio) acetic acid (I-320)

A solution of 1-(2-aminoethyl)-4-methylpiperazine-2,5-dione, I-319 (280 mg, 1.27 mmol) and thiodiglycolic anhydride (300 mg, 1.8 equiv.) in 5 mL of DCM was stirred for 2 hours, and then was concentrated in vacuo to give the crude product as a pale oil. The crude product was purified by preparative HPLC (Mobile Phase: MeCN/H₂O (0.1% formic acid) Gradient: 5-15% MeCN) to give 50 mg of carboxylic acid I-320 as a white solid, yield: 13%, ¹H NMR (400 MHz, CDCl₃) δ 7.34 (s, 1H), 4.14 (s, 2H), 4.06 (s, 2H), 3.64 (dd, 2H), 3.55 (dd, 2H), 3.36 (s, 2H), 3.25 (s, 2H), 3.00 (s, 3H).

### The Synthesis of I-325 Involved 5 Steps as Depicted in the Following Scheme 114

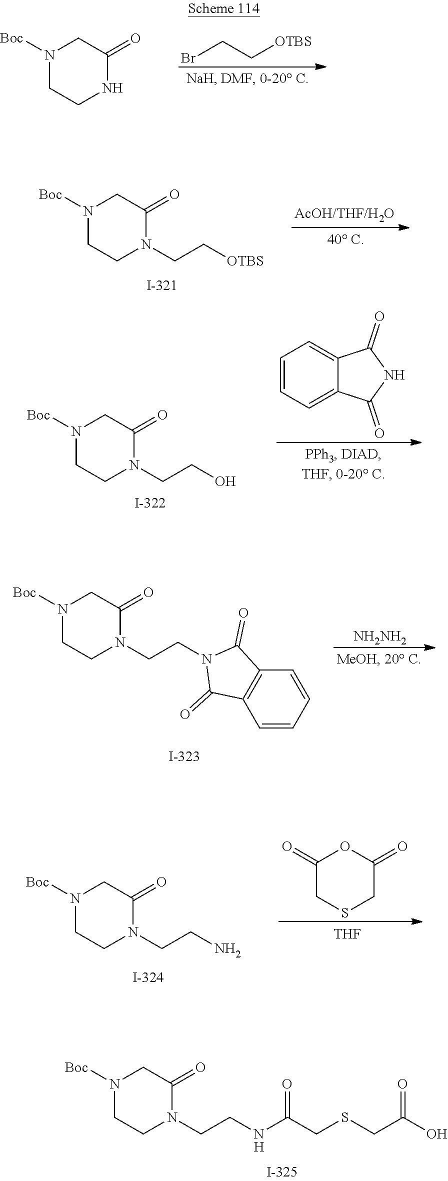

### Step 1: tert-butyl 4-(2-((tert-butyldimethylsilyl)oxy)ethyl)-3-oxopiperazine-1-carboxylate (I-321)

To a solution of tert-butyl 3-oxopiperazine-1-carboxylate (cas: 76003-29-7, 2 g, 10 mmol) in DMF (20 mL) that had been pre-cooled in an ice-water bath was added NaH (800 mg, 2.0 equiv.) in portions under N₂. The mixture was stirred for 0.5 hours. (2-bromoethoxy)(tert-butyl)dimethylsilane (cas: 86864-60-0, 5.3 mL, 2.5 equiv.) was added to the solution at 0° C. The reaction mixture was stirred at 25° C. for 12 hours. The reaction mixture was then diluted with water (50 mL) and extracted with EtOAc (3×80 mL). The combined organic extracts was then washed with water (50 mL), brine (50 mL), and dried over Na₂SO₄, then concentrated under reduced pressure to a residue. The residue was purified by silica gel chromatography to afford ketopiperazine I-321 as a pale oil (1.8 g, 50% yield, pale oil). MS (ESI, pos. ion) m/z: 381 (M+23).

### Step 2: tert-butyl 4-(2-hydroxyethyl)-3-oxopiperazine-1-carboxylate (I-322)

A solution of tert-butyl 4-(2-((tert-butyldimethylsilyl)oxy)ethyl)-3-oxopiperazine-1-carboxylate, I-321 (1.8 g, 5 mmol) in AcOH/THF/H₂O (40 mL/13 mL/13 mL) was stirred at 40° C. for 2 hours. Upon completion of the reaction, the solvent was removed under reduced pressure. The crude product was purified by silica gel chromatography to give the alcohol I-322 (0.95 g, 78%, pale oil). MS (ESI, pos. ion) m/z: 267 (M+23).

### Step 3: tert-butyl 4-(2-(1,3-dioxoisoindolin-2-yl)ethyl)-3-oxopiperazine-1-carboxylate (I-323)

To a solution of tert-butyl 4-(2-hydroxyethyl)-3-oxopiperazine-1-carboxylate, I-322 (0.82 g, 3.4 mmol) in THF (8 mL) was added phthalimide (cas: 85-41-6, 0.75 g, 1.5 equiv.), PPh₃ (1.6 g, 1.8 equiv.) in sequence at 0° C. under N₂. Then DIAD (1.4 mL, 2.0 equiv.) was added dropwise. The mixture was then stirred at 25° C. for 1.5 hours. After LC-MS showed the reaction went to completion, the mixture solution was filtered. The filtrate was concentrated under reduced pressure to afford the crude product. The crude product was purified by silica gel chromatography to give phthalimide I-323 as a white solid (purity: 40%, 2.8 g, 88% yield). MS (ESI, pos. ion) m/z: 396 (M+23).

### Step 4: tert-butyl 4-(2-aminoethyl)-3-oxopiperazine-1-carboxylate (I-324)

To a solution of tert-butyl 4-(2-(1,3-dioxoisoindolin-2-yl)ethyl)-3-oxopiperazine-1-carboxylate, I-323 (0.58 g, ca. 0.77 mmol) in MeOH (8 mL) that had been pre-cooled in an ice-water bath was added NH₂NH₂ (85% in H₂O, 0.1 mL, 2 equiv.). The reaction mixture was allowed to warm to ambient temperature and then stirred for 18 hours. Upon completion of the reaction, the slurry was filtered and the filtrate was concentrated to afford a pale oil. The crude product, I-324 was used directly for next step. MS (ESI, pos. ion) m/z: 266 (M+23).

### Step 5: 2-((2-((2-(4-(tert-butoxycarbonyl)-2-oxopiperazin-1-yl)ethyl)amino)-2-oxoethyl)thio) acetic acid (I-325)

A solution of tert-butyl 4-(2-aminoethyl)-3-oxopiperazine-1-carboxylate, I-324 (crude product, ca. 0.77 mmol) and 1,4-thiodiglycolic anhydride (100 mg, 1.0 equiv.) in THF (5 mL) was stirred for 5 min and concentrated under reduced pressure. The crude product was purified by preparative HPLC (Mobile Phase: MeCN/H₂O (0.1% formic acid), Gradient: 20-30% MeCN) to afford carboxylic acid I-325 as a white solid (54 mg, 19% yield). MS (ESI, pos. ion) m/z: 398.2 (M+23). ¹H NMR (400 MHz, CDCl₃) δ 4.14 (s, 2H), 3.67 (dd, 4H), 3.59-3.50 (m, 2H), 3.44 (d, 2H), 3.35 (s, 2H), 3.27 (s, 2H), 1.47 (s, 9H).

### The Synthesis of I-328 Involved 3 Steps as Depicted in the Following Scheme 115

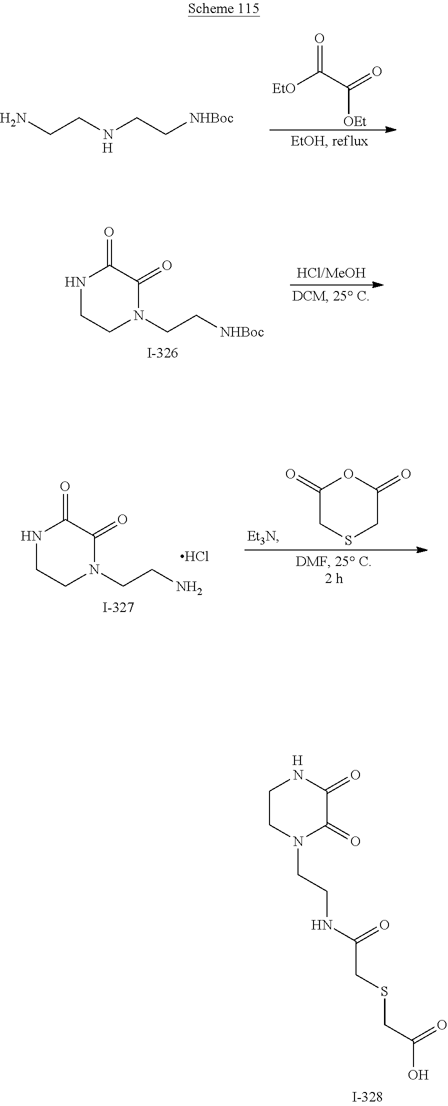

The chemical structure of the target compound was confirmed by 1HNMR and LC-MS with the purity >95% (HPLC 214 & 254 nm).

### 2-((2-((2-(2,3-dioxopiperazin-1-yl)ethyl)amino)-2-oxoethyl)thio) acetic acid (I-328)

### Step 1. tert-butyl (2-(2,3-dioxopiperazin-1-yl)ethyl)carbamate (I-326)

To a solution of tert-butyl (2-((2-aminoethyl)amino)ethyl)carbamate (cas: 193206-49-4, 1 g, 5 mmol) in EtOH (50 mL) was added diethyl oxalate (cas: 95-92-1, 0.73 g, 1.0 equiv.). The reaction mixture was heated to reflux for 18 hours, then concentrated under r educed pressure. The residue obtained was purified by silica gel chromatography to provide carbamate I-326 as a colorless foam. (330 mg, 26% yield). MS (ESI, pos. ion) m/z: 28 0 (M+23).

### Step 2. 1-(2-aminoethyl)piperazine-2,3-dione hydrochloride (I-327)

To a solution of tert-butyl (2-(2,3-dioxopiperazin-1-yl)ethyl)carbamate, I-326 (280 mg 1.1 mmol) in DCM (20 ml) was added HCl/MeOH (5 mL). The reaction mixture was then stirred at ambient temperature for 1 hour, then concentrated under reduced pressure to afford 210 mg of the crude product I-327, which was directly used without further purification. MS (ESI, pos. ion) m/z: 158 (M+1).

### Step 3. 2-((2-((2-(2,3-dioxopiperazin-1-yl)ethyl)amino)-2-oxoethyl)thio) acetic acid (I-328)

To a solution of 1-(2-aminoethyl)piperazine-2,3-dione hydrochloride, I-327 (210 mg, 1.1 mmol) and thiodiglycolic anhydride (cas: 3261-87-8, 144 mg, 1.1 equiv.) in DMF (10 mL) was added TEA (222 mg, 2.2 equiv.). The reaction mixture was stirred at ambient temperature for 2 hours and then was concentrated under reduced pressure to afford the crude product. The crude product was purified by preparative HPLC (Mobile Phase: MeCN/H₂O (with 0.1% TFA)) to give afford acid I-328 (56 mg, 18% yield) as a white solid). MS (ESI, pos. ion) m/z: 290 (M+1). ¹H NMR (400 MHz, DMSO) δ 8.54 (s, 1H), 8.18 (t, 1H), 3.55-3.45 (m, 2H), 3.41 (t, 2H), 3.32 (d, 2H), 3.25 (dd, 2H), 3.18 (s, 2H).

The synthesis of I-332 involved 4 steps as depicted in the following Scheme 116.

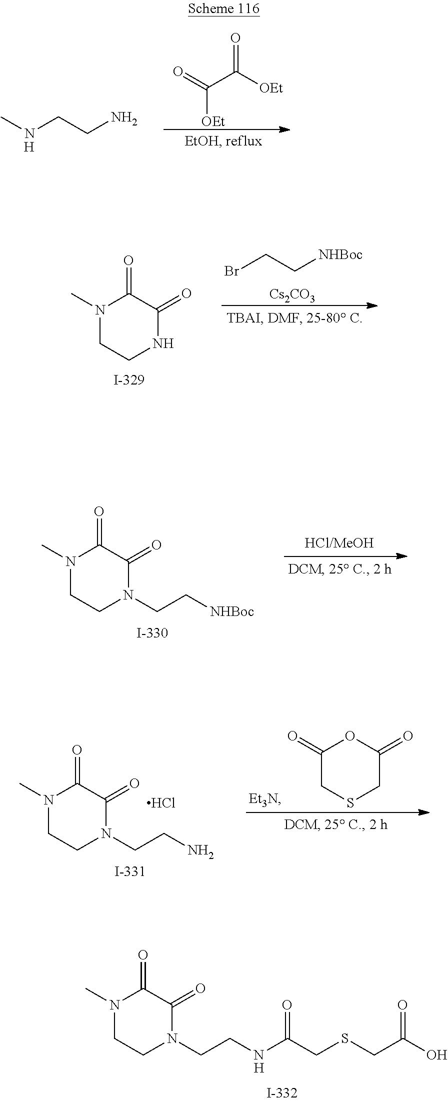

The chemical structure of the target compound was confirmed by ¹H NMR and LC-MS with the purity >95% (HPLC 214 & 254 nm).

### (S)-2-((2-((2-(2-(hydroxymethyl)-5-oxopyrrolidin-1-yl)ethyl)amino)-2-oxoethyl)thio) acetic acid (I-332)

### Step 1: 1-Methylpiperazine-2,3-dione (I-329)

To a solution of N-methylethylene-1,2-diamine (cas: 109-81-9, 1 g, 13.5 mmol) in EtOH (80 mL) was added diethyl oxalate (cas: 95-92-1, 2 g, 5 mmol). The reaction was heated to reflux for 18 hours, then concentrated under reduced pressure. The residue obtain ed was purified by silica gel chromatography to provide (1.4 g, 80% yield) of I-329 as a colorless foam. MS ESI, pos. ion) m/z: 129 (M+1).

### Step 2: Tert-butyl (2-(4-methyl-2,3-dioxopiperazin-1-yl)ethyl)carbamate (I-330)

To a slurry of 1-methylpiperazine-2,3-dione, I-329 (1 g, 7.8 mmol), Cs₂CO₃ (12.7 g, 5.0 equiv.) and TBAI (288 mg, 0.1 equiv.) in DMF (50 mL) was added tert-butyl (2-bromoethyl)carbamate (3.5 g, 2.0 equiv.). The reaction mixture was heated to 80° C. for 24 hours. The reaction mixture was then concentrated and the residue obtained was purified by silica gel chromatography to provide 1.4 g (67% yield) of carbamate I-330 as a colorless oil. MS (ESI, pos. ion) m/z: 294 (M+23).

### Step 3: 1-(2-Aminoethyl)-4-methylpiperazine-2,3-dione hydrochloride (I-331)

To a solution of tert-butyl (2-(4-methyl-2,3-dioxopiperazin-1-yl)ethyl)carbamate, I-330 (100 mg, 0.37 mmol) in DCM (12 mL) was added HCl/MeOH (2 mL). The reaction was then stirred at ambient temperature for 1 hour. The reaction mixture was then concentrated and the crude product (77 mg, colorless oil) obtained was directly used without purification. MS (ESI, pos. ion) m/z: 172 (M+1).

### Step 4: 2-((2-((2-(4-Methyl-2,3-dioxopiperazin-1-yl)ethyl)amino)-2-oxoethyl)thio) acetic acid (I-332)

To a solution of 1-(2-aminoethyl)-4-methylpiperazine-2,3-dione hydrochloride, I-331 (77 mg, 0.37 mmol) and thiodiglycolic anhydride (49 mg, 1.0 equiv.) in DCM (10 mL) was added TEA (75 mg, 0.74 mmol). The reaction mixture was stirred at ambient temperature for 1 hour and then was concentrated under reduced pressure to give the crude product. The crude product was purified by preparative HPLC (Mobile Phase: MeCN/H₂O (0.1% TFA)) to afford acid I-332 (40 mg, 37% yield). MS (ESI, pos. ion) m/z: 304 (M+1). ¹H NMR (400 MHz, DMSO) δ 8.21 (s, 1H), 3.52 (d, J=4.9 Hz, 4H), 3.39 (t, J=5.5 Hz, 2H), 3.31 (s, 2H), 3.26 (d, J=5.5 Hz, 2H), 3.17 (s, 2H), 2.91 (s, 3H).

### The Synthesis of Compound I-335 Involved 3 Steps as Depicted in the Following Scheme 117

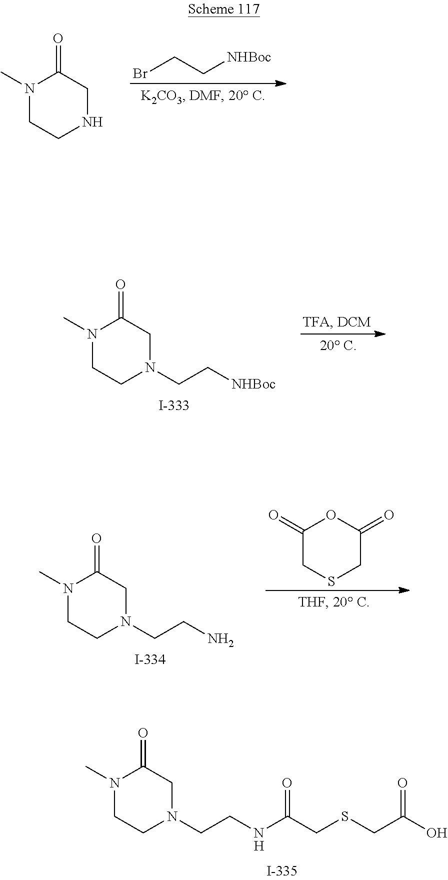

The chemical structure of the target compound was confirmed by 1HNMR and LC-MS with the purity >95% (HPLC 214 & 254 nm).

### Step 1: Tert-butyl (2-((3-chloropropyl)sulfonamido)ethyl)carbamate (I-333)

A solution of 1-methylpiperazin-2-one (cas: 5625-67-2, 1 g, 9.99 mmol), tert-butyl (2-bromoethyl)carbamate (cas: 39684-80-5, 2 g, 0.9 equiv.) in anhydrous DMF (15 mL) was added K₂CO₃ (8.28 g, 6.0 equiv.). The reaction mixture was stirred for 8 hours at ambient temperature. Once LC-MS analysis indicated reaction completion, the reaction was quenched with H₂O (20 mL) and the aqueous phase was extracted with EtOAc (20 mL×3). The combined organic extracts was concentrated under reduced pressure to give the crude product. The crude product was purified by silica gel chromatography (Petroleum ether:EtOAc=1:10) to provide carbamate I-333 as a pale oil (0.65 g, 22%). MS (ESI, pos. ion) m/z: 258 (M+1).

### Step 2:4-(2-Aminoethyl)-1-methylpiperazin-2-one (I-334)

A solution of tert-butyl (2-(4-methyl-3-oxopiperazin-1-yl)ethyl)carbamate, I-333 (0.25 g, 0.97 mmol) in DCM (2 mL) that had been pre-cooled in an ice-water bath was added a solution of TFA/DCM (1:1 v/v, 2 mL). The reaction mixture was then stirred at ambient temperature for 2 hours. When the LC-MS showed the reaction went to completion, the reaction mixture was concentrated in vacuo to afford the crude amine, I-334 (pale oil, 200 mg). The crude product was used directly for next step. MS (ESI, pos. ion) m/z: 158 (M+1).

### Step 3: 2-((2-((2-(4-Methyl-3-oxopiperazin-1-yl)ethyl)amino)-2-oxoethyl)thio) acetic acid (I-335)

A solution of 4-(2-aminoethyl)-1-methylpiperazin-2-one, I-334 (0.2 g, 1.27 mmol) and thiodiglycolic anhydride (0.185 g, 1.1 equiv.) in THF (5 mL) was stirred at ambient temperature for 4 hours, and then the reaction mixture was concentrated to give the crude product. The crude product was purified by preparative HPLC (Mobile Phase: MeCN/H₂O (0.1% TFA)) to afford the acid I-335 as a pale oil (50 mg, 2% yield). MS (ESI, pos. ion) m/z: 290.1 (M+1). ¹H NMR (400 MHz, DMSO) δ 8.26 (s, 1H), 3.75 (s, 2H), 3.48 (s, 3H), 3.44-3.36 (m, 5H), 3.26 (s, 2H), 3.12 (m, 2H), 2.87 (s, 3H).

### The Synthesis of Compound I-338 Involved 3 Steps as Depicted in the Following Scheme 118

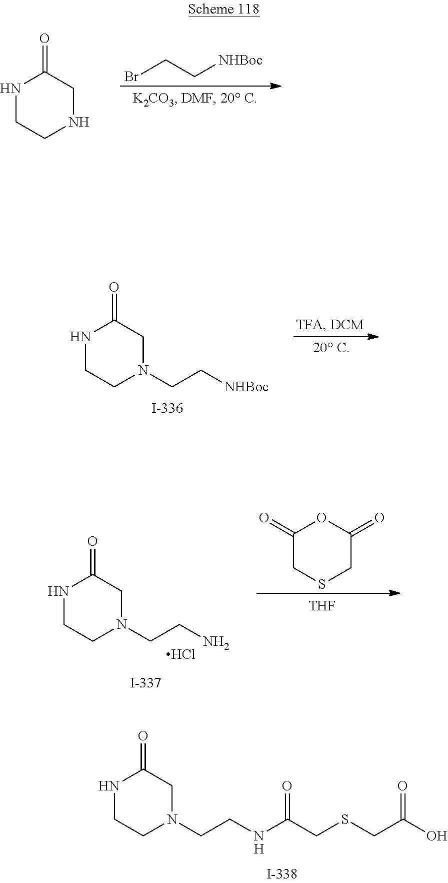

### 2-((2-oxo-2-((2-(3-oxopiperazin-1-yl)ethyl)amino)ethyl)thio)acetic acid (I-338)

The synthetic route for I-338 was similar to that of I-335 and commenced with piperazin-2-one (cas: 5625-67-2). 2-((2-oxo-2-((2-(3-oxopiperazin-1-yl)ethyl)amino)ethyl)thio)acetic acid (pale oil, 50 mg, 15% yield) was isolated by preparative HPLC (Mobile Phase: MeCN/H₂O (with 0.1% TFA)). MS (ESI, pos. ion) m/z: 276.1 (M+1). ¹H NMR (400 MHz, MeOD) δ 3.82 (s, 2H), 3.60-3.53 (m, 4H), 3.47 (m, 2H), 3.38 (s, 2H), 3.35 (s, 2H), 3.23 (m, 2H).

The synthesis of I-343 is depicted in Scheme 119.

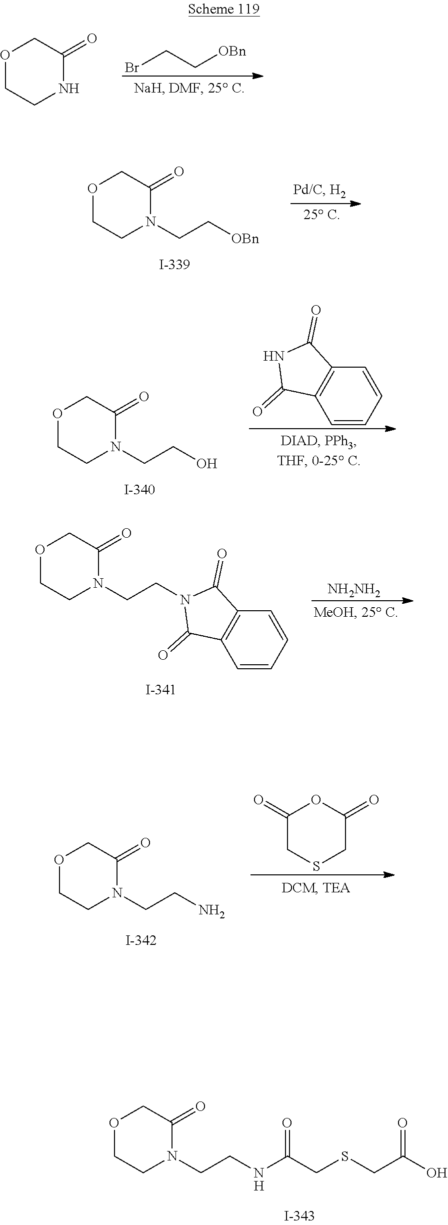

The chemical structure of the target compound was confirmed by ¹H NMR and LC-MS with the purity >95% (HPLC 214 & 254 nm).

### Step 1:4-(2-(benzyloxy)ethyl) morpholin-3-one (I-339)

To a solution of morpholin-3-one (cas: 109-11-5, 2 g, 19.8 mmol) in anhydrous DMF (25 mL) was added NaH (60% dispersion in mineral oil, 1.98 g, 2.5 equiv.) at 0° C. under N₂. The reaction mixture was stirred for 30 minutes and then ((2-bromoethoxy)methyl)benzene (cas: 1462-37-9, 1.98 g, 2.5 equiv.) was added to the reaction mixture, which was then allowed to warm to ambient temperature. After 12 hours, analysis by LC-MS indicated completion of the reaction. The reaction mixture was quenched with H₂O (20 mL) and extracted with EtOAc (20 mL×3). The combined organic extracts were concentrated under reduced pressure to give the crude product, which was purified by silica gel chromatography (Petroleum ether:EtOAc=1:4) to provide morpholinone I-339 as a pale oil (2.3 g, 49% yield). MS (ESI, pos. ion) m/z: 236 (M+1).

### Step 2:4-(2-hydroxyethyl) morpholin-3-one (I-340)

To a solution of 4-(2-(benzyloxy)ethyl) morpholin-3-one, I-339 (2.35 g, 9.79 mmol) in MeOH (10 mL) was added Pd/C (230 mg) quickly under H₂ (1 atm). The reaction mixture was then stirred at ambient temperature for 48 hours, when analysis by LC-MS showed the reaction was complete. The reaction mixture was filtered and the filtrate was concentrated under reduced pressure to afford the crude product, I-340 (pale oil, 1.35 g, 93% yield). The crude product was used directly in next step. MS (ESI, pos. ion) m/z: 146 (M+1).

### Step 3:2-(2-(3-oxomorpholino)ethyl)isoindoline-1,3-dione (I-341)

A solution of 4-(2-hydroxyethyl) morpholin-3-one, I-340 (1.37 g, 9.45 mmol), phthalimide (cas: 85-41-6, 1.53 g, 1.1 equiv.) and PPh₃ (3.72 g, 1.5 equiv.) in anhydrous THF (30 mL) was stirred at 0° C. for 0.5 hours. Then DIAD (5.73 g, 3.0 equiv.) was added and the reaction mixture was allowed to warm to ambient temperature and stirred for 7 hours. The reaction mixture was concentrated and the residue obtained was purified by silica gel chromatography (Petroleum ether:EtOAc=1:5) to provide I-341 as a pale oil (2.5 g, 50% purity, 50% yield). MS (ESI, pos. ion) m/z: 275 (M+1).

### Step 4:4-(2-aminoethyl) morpholin-3-one (I-342)

A solution of 2-(2-(3-oxomorpholino)ethyl)isoindoline-1,3-dione, I-341 (1.25 g 4.56 mmol) in MeOH (10 mL) was added hydrazine hydrate (80% aqueous solution, 0.291 g, 1.5 equiv.). The reaction mixture was stirred for 12 hours at ambient temperature, then filtered and the filtrate was then concentrated to give the crude product, I-342 (1 g), which was taken forward to the next step without further purification. MS (ESI, pos. ion) m/z: 145 (M+1).

### Step 5: 2-((2-oxo-2-((2-(3-oxomorpholino)ethyl)amino)ethyl)thio)acetic acid (I-343)

A solution of 4-(2-aminoethyl) morpholin-3-one, I-342 (1 g, 6.94 mmol) and thiodiglycolic anhydride (cas: 3261-87-8, 1.09 g, 1.2 equiv.) in 10 mL of DCM was stirred for 2 hours, and then concentrated under reduced pressure to afford a residue, which was purified by preparative HPLC (Mobile Phase: MeCN/H₂O (with 0.1% Formic Acid), Gradient: 5-15% MeCN) to obtain I-343 (50 mg, 3% yield) as a pale oil. MS (ESI, pos. ion) m/z: 277 (M+1). ¹H NMR (400 MHz, DMSO) § 12.65 (s, 1H), 8.14 (m, 1H), 4.00 (s, 2H), 3.82-3.75 (m, 2H), 3.40-3.34 (m, 6H), 3.24 (m, 2H), 3.18 (s, 2H).

### The Synthesis of I-348 Involved 5 Steps as Depicted in the Following Scheme 120

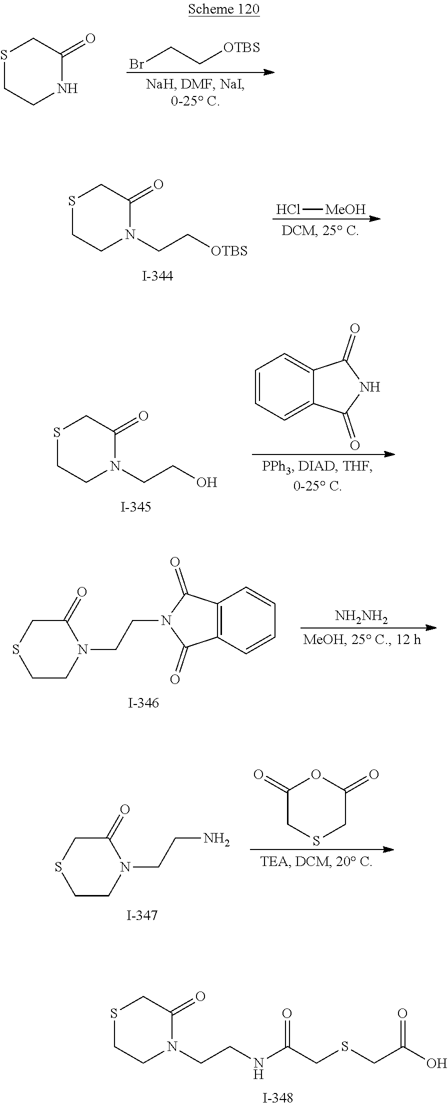

### Step 1: 4-(2-((tert-butyldimethylsilyl)oxy)ethyl)thiomorpholin-3-one (I-344)

To a solution of thiomorpholin-3-one (cas: 20196-21-8, 1.17 g, 10 mmol) in anhydrous DMF (10 mL) was added sodium hydride (cas: 7646-69-7, 1.20 g, 30 mmol, 3.0 equiv.) in ice bath under N₂. After 30 minutes, (2-bromoethoxy)(tert-butyl)dimethylsilane (cas: 86864-60-0, 3.59 g, 1.5 equiv.) was added to the solution at 0° C. . . . The reaction mixture was allowed to warm to ambient temperature and stirred for 5 hours. Once complete, the reaction was quenched with water (5 mL), extracted with EtOAc (3×500 mL). The combined organic extracts were then washed with water (50 mL), brine (50 mL), and dried with Na₂SO₄, then concentrated under reduced pressure to a residue, which was purified by silica gel chromatography to afford I-344 (1.40 g, 54% yield) as a pale yellow oil. MS (ESI, pos. ion) m/z: 276.1 (M+1).

### Step 2: 4-(2-hydroxyethyl)thiomorpholin-3-one (1-345)

To a solution of 4-(2-((tert-butyldimethylsilyl)oxy)ethyl)thiomorpholin-3-one, I-344 (1.40 g, 5.09 mmol) in DCM (15 mL) that had been pre-cooled in an ice-water bath was added HCl in MeOH (30% v/v, 10 mL) under N₂. The reaction mixture was stirred for 3 hours. Upon completion of the reaction, the resulting solution was concentrated under reduced pressure to a residue. The residue was purified by silica gel chromatography to afford I-345 (0.71 g, 87% yield) as a pale yellow oil. MS (ESI, pos. ion) m/z: 162.1 (M+1).

### Step 3: 2-(2-(3-oxothiomorpholino)ethyl)isoindoline-1,3-dione (I-346)

To a solution of 4-(2-hydroxyethyl)thiomorpholin-3-one, I-345 (0.71 g, 4.41 mmol) in anhydrous THF (10 mL) at 0-5° C. was added phthalimide (cas: 85-41-6, 0.72 g, 1.1 equiv.) and PPh₃ (1.74 g, 1.5 equiv.) under N₂. After 30 minutes, DIAD (2.67 g 3.0 equiv.) was added. The reaction mixture was allowed to warm to ambient temperature and stirred for 12 hours. Upon completion of the reaction, the reaction mixture was concentrated under reduced pressure to a residue, which was purified by silica gel chromatography to afford imide I-346 (0.90 g, 70% yield, about 50% purity, contaminated with PPh₃₀) as a white solid. MS (ESI, pos. ion) m/z: 291.1 (M+1).

### Step 4: 4-(2-aminoethyl)thiomorpholin-3-one (I-347)

To a solution of 2-(2-(3-oxothiomorpholino)ethyl)isoindoline-1,3-dione, I-346 (0.9 g, 1.24 mmol) in MeOH (10 mL) at 0-5° C. was added NH₂NH₂ (85% in H₂O, 0.14 g, 1.2 equiv.). The reaction mixture was allowed to warm to ambient temperature and stirred for 12 hours. Upon completion of the reaction, the reaction mixture was concentrated under reduced pressure. The residue obtained was purified by reversed-phase chromatography to afford the amine I-347 as a pale oil (0.41 g, 82% yield, about 40% purity). MS (ESI, pos. ion) m/z: 162.1 (M+1).

### Step 5: 2-((2-oxo-2-((2-(3-oxothiomorpholino)ethyl)amino)ethyl)thio)acetic acid (I-348)

A solution of 4-(2-aminoethyl)thiomorpholin-3-one, I-347 (0.41 g, 2.56 mmol) and thiodiglycolic anhydride (cas: 3261-87-8, 0.405 g, 3.07 mmol, 1.2 equiv.) in DCM (5 ml) and DMF (2.0 ml) was stirred for 16 hours at 25° C. Upon completion of the reaction, the mixture was concentrated by vacuum to afford a residue, which was purified by preparative HPLC (Mobile Phase: MeCN/H₂O (0.1% TFA), Gradient: 20-30% MeCN) to obtain 22.8 mg (3% yield) of I-348 as a pale oil. MS (ESI, pos. ion) m/z: 293.1 (M+1). ¹H NMR (400 MHz, CDCl₃): ¹H NMR (400 MHz, CDCl₃): 7.41 (s, 1H), 3.70 (ddd, 4H), 3.55 (d, 2H), 3.39 (d, 4H), 3.28 (s, 2H), 2.93-2.89 (m, 2H).

### The Synthesis of I-351 Involved 3 Steps from the Previously Described Intermediate I-346 as Depicted in the Following Scheme 121

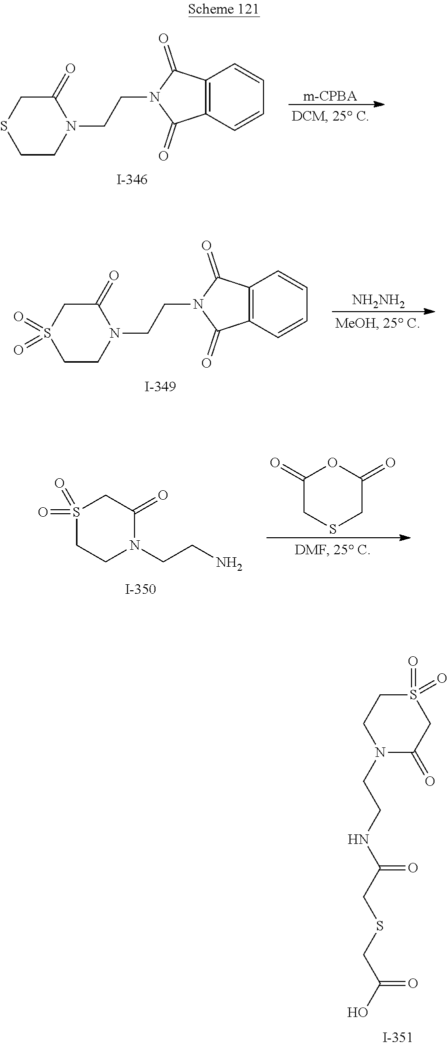

### Step 1: 2-(2-(1,1-dioxido-3-oxothiomorpholino)ethyl)isoindoline-1,3-dione(I-349)

To a solution of 2-(2-(3-oxothiomorpholino)ethyl)isoindoline-1,3-dione, I-346 (2.3 g, purity 50%, 7.9 mmol) in DCM (10 mL) at 0-5° C. was added m-chloroperbenzoic acid (4.08 g, 3.0 equiv.). The mixture was allowed to warm to ambient temperature and stirred for 12 hours. Upon completion of the reaction, the reaction mixture was washed with saturated aqueous Na₂SO₃ (10 ml), saturated aqueous NaHCO₃ (10 ml), brine (10 ml), dried, and concentrated under reduced pressure. The residue was purified by silica gel chromatography to afford sulfone I-349 as a white solid (0.3 g, 23% yield, about 80% purity). MS (ESI, pos. ion) m/z: 323.1 (M+1).

### Step 2: 4-(2-aminoethyl)thiomorpholin-3-one 1,1-dioxide (I-350)

To a solution of 2-(2-(1,1-dioxido-3-oxothiomorpholino)ethyl)isoindoline-1,3-dione, I-349 (0.3 g, 0.93 mmol) in MeOH (10 mL) was added hydrazine (85% solution in H₂O, 0.042 g, 1.2 equiv.). The reaction mixture was stirred for 12 hours. Upon completion of the reaction, the reaction mixture was concentrated under reduced pressure to a residue, which was purified by reversed-phase chromatography to afford amine I-350 (0.1 g, 56% yield, about 80% purity) as a pale oil. MS (ESI, pos. ion) m/z: 193.1 (M+1).

### Step 3: 2-((2-oxo-2-((2-(3-oxothiomorpholino)ethyl)amino)ethyl)thio) acetic acid (I-351)

A solution of 4-(2-aminoethyl)thiomorpholin-3-one 1,1-dioxide, I-350 (0.1 g, 0.52 mmol) and thiodiglycolic anhydride (cas: 3261-87-8, 0.082 g, 0.62 mmol, 1.2 equiv.) in DMF (4 mL) was stirred for 3 hours at 25° C. The reaction mixture was then concentrated under reduced pressure to afford a pale oil. The crude product was purified by preparative HPLC (Mobile Phase: MeCN/H₂O (0.1% TFA), Gradient: 1-3% MeCN) to afford 63.4 mg (38% yield) of acid I-351 as a pale oil. MS (ESI, pos. ion) m/z: 325.1 (M+1). ¹H NMR (400 MHz, D20) δ 4.19 (s, 1H), 3.87 (dd, 1H), 3.59-3.53 (m, 2H), 3.41-3.37 (m, 1H), 3.32 (s, 2H), 3.25 (s, 2H).

### The Synthesis of I-356 Involved 5 Steps as Depicted in the Following Scheme 122

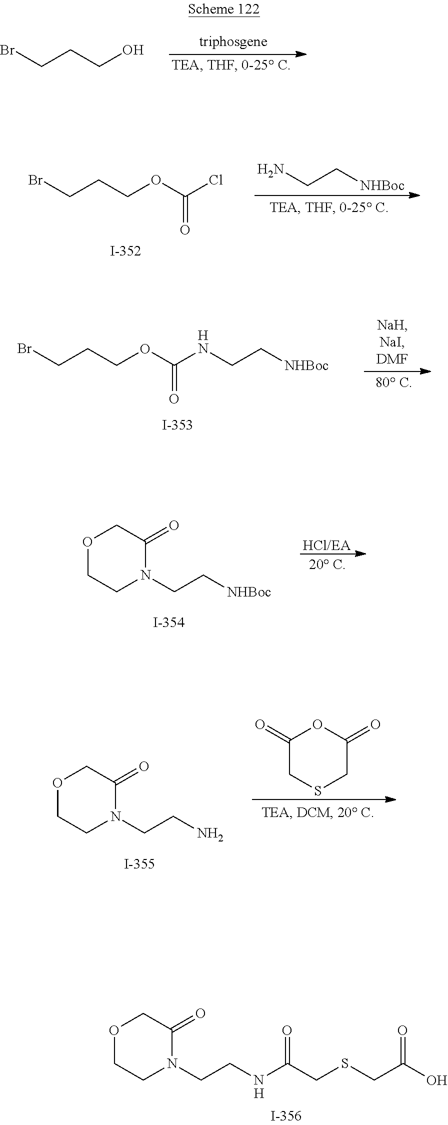

### Step 1:3-bromopropyl carbonochloridate (I-352)

To a solution of 3-bromopropan-1-ol (cas: 627-18-9, 600 mg, 4.3 mmol) in 15 mL of THF that had been pre-cooled to 0° C. was added TEA (0.9 mL) and a solution of triphosgene (600 mg, 0.5 equiv.) in 5 mL of THF under N₂. The reaction mixture was monitored by LC-MS (the reaction aliquot was treated with BnNH₂ prior to analysis). After 30 minutes, the reaction mixture was used directly in next step. MS (ESI, pos. ion) m/z: 272.2 (M+1).

### Step 2:3-bromopropyl tert-butyl ethane-1,2-diyldicarbamate (I-353)

To the reaction mixture from previous step, was added tert-butyl (2-aminoethyl)carbamate (1 g, 1.5 equiv.) in one portion. The reaction mixture was stirred at 25° C. for 15 hours. Upon completion of the reaction, the reaction was quenched with water (50 mL), and extracted with EtOAc (3×50 mL). The combined organic extracts were concentrated to a residue, which was purified by silica gel chromatography (Petroleum ether:EtOAc=1:1) to afford 1.2 g (86% yield, 2 steps) of carbamate I-353 as a white solid. MS (ESI, pos. ion) m/z: 347.2 (M+23).

### Step 3: tert-butyl (2-(2-oxo-1,3-oxazinan-3-yl)ethyl)carbamate (I-354)

A solution of 3-bromopropyl tert-butyl ethane-1,2-diyldicarbamate, I-353 (1.1 g 3.4 mmol), NaH (360 mg, 2.5 equiv., 60% dispersion in mineral oil), NaI (350 mg, 1 equiv.) in DMF (60 mL) was heated to 80° C. for 2 hours. On completion, the reaction mixture was quenched with water (30 mL) and extracted with EtOAc (3×30 mL). The combined organic extracts were concentrated to a residue, which was purified by silica gel chromatography (EtOAc), to afford oxazinone I-354 (100 mg, 12%) as a pale oil. MS (ESI, pos. ion) m/z: 267.2 (M+23).

### Step 4: 3-(2-aminoethyl)-1,3-oxazinan-2-one (I-355)

A solution of tert-butyl (2-(2-oxo-1,3-oxazinan-3-yl)ethyl)carbamate (100 mg 0.4 mmol) in HCl (2M in EtOAc, 3 mL) was stirred at 25° C. for 2 hours. Upon completion of the reaction, the reaction mixture was concentrated and purified by preparative HPLC (Mobile Phase: MeCN/H₂O (0.1% TFA) to afford amine I-355 (30 mg, 50% yield) as a pale oil. MS (ESI, pos. ion) m/z: 145.2 (M+1).

### Step 5: 2-((2-oxo-2-((2-(2-oxo-1,3-oxazinan-3-yl)ethyl)amino)ethyl)thio)acetic acid (I-356)

To a stirred solution of 3-(2-aminoethyl)-1,3-oxazinan-2-one, I-355 (30 mg, 0.2 mmol) in DCM (2 mL), was added thiodiglycolic anhydride (cas: 3261-87-8, 27 mg, 1 equiv.), TEA (4 drops) at 25° C. The reaction mixture was stirred for 2 hours, and then concentrated and purified by preparative HPLC (Mobile Phase: MeCN/H₂O (0.1% TFA), gradient: 2-8% MeCN) to afford acid I-356 (15 mg, 27%) as a pale oil. MS (ESI, pos. ion) m/z: 277.2 (M+1). ¹H NMR (400 MHz, CDCl₃) δ 7.38 (s, 1H), 4.33-4.28 (m, 2H), 3.63-3.58 (m, 2H), 3.57-3.52 (m, 2H), 3.45 (t, 2H), 3.37 (s, 2H), 3.27 (s, 2H), 2.11-2.07 (m, 2H).

### The Synthesis of I-363 Involved 7 Steps as Depicted in the Following Scheme 123

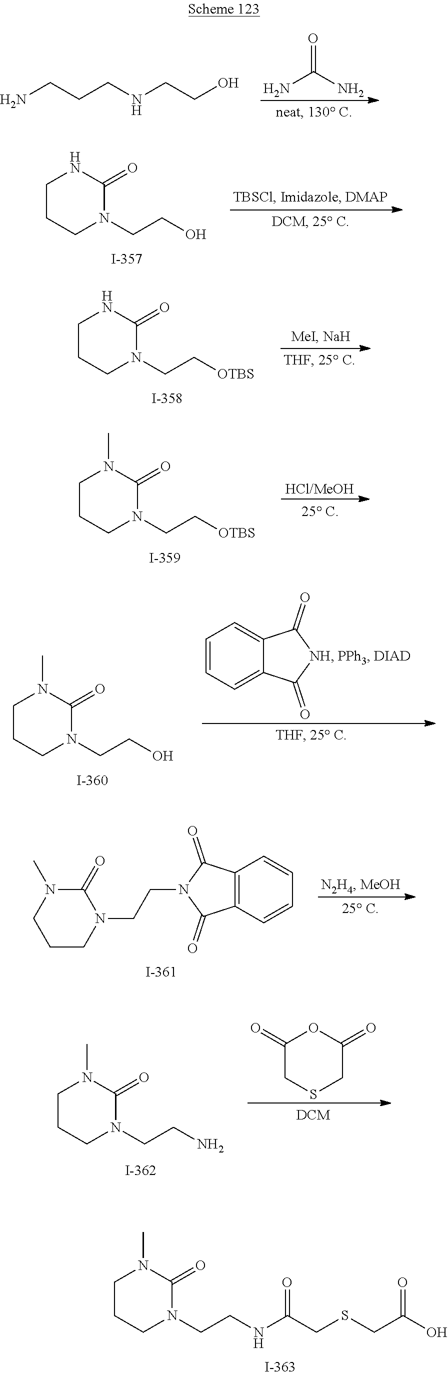

### 2-((2-((2-(3-methyl-2-oxotetrahydropyrimidin-1(2H)-yl)ethyl)amino)-2-oxoethyl)thio) acetic acid (I-363)

### Step 1:1-(2-hydroxyethyl)tetrahydropyrimidin-2(1H)-one (I-357)

2-((3-aminopropyl)amino) ethan-1-ol (5.9 g, 50 mmol, cas #: 4461-39-6) and urea (4.5 g, 1.5 equiv., cas #: 57-13-6) in a sealed tube was heated at 130° C. for 10 hours. After reaction completion, the residue was purified by silica gel column chromatography (20% MeOH in dichloromethane) to afford alcohol I-357 (1 g, 14% yield) as a white solid. MS (ESI, pos. ion) m/z: 145.1 (M+1).

### Step 2:1-(2-((tert-butyldimethylsilyl)oxy)ethyl)tetrahydropyrimidin-2(1H)-one (I-358)

To a solution of 1-(2-hydroxyethyl)tetrahydropyrimidin-2(1H)-one, I-357 (2.6 g, 18 mmol), DMAP (220 mg, 0.1 equiv.) and imidazole (2.5 g, 2.0 equiv.) in 30 mL of DCM was added TBSCl (2.7 g, 1.0 equiv.). The reaction mixture was stirred at ambient temperature for 16 hours, then diluted with 50 ml of water and extracted with DCM (30 mL×3). The combined organic extracts were dried over Na₂SO₄ and concentrated in vacuo to afford 3.1 g (67% yield) of silyl ether I-358 as a pale oil. MS (ESI, pos. ion) m/z: 259.1 (M+1).

### Step 3: 1-(2-((tert-butyldimethylsilyl)oxy)ethyl)-3-methyltetrahydropyrimidin-2(1H)-one (I-359)

To a solution of 1-(2-((tert-butyldimethylsilyl)oxy)ethyl)tetrahydropyrimidin-2(1H)-one, I-358 (2.1 g, 8 mmol) and NaH (960 mg, 5.0 equiv.) in THF (30 mL) at 0° C. was added MeI (3.4 g, 3.0 equiv.). The reaction mixture was stirred at ambient temperature for 1 hour, then the solvent was removed in vacuo, and the residue obtained was purified by column chromatography (DCM) to afford silyl ether I-359 (1.1 g, 50% yield) as a white solid. MS (ESI, pos. ion) m/z: 273.1 (M+1).

### Step 3:1-(2-((tert-butyldimethylsilyl)oxy)ethyl)-3-methyltetrahydropyrimidin-2(1H)-one (I-360)

To a solution of 1-(2-((tert-butyldimethylsilyl)oxy)ethyl)-3-methyltetrahydropyrimidin-2(1H)-one, I-359 (1.1 g, 4 mmol) in MeOH (10 mL) at 0-5° C. was added HCl in MeOH (30% v/v, 10 mL) under N₂. The reaction mixture was stirred for 0.5 hours, then concentrated under reduced pressure to afford alcohol I-360 (600 mg 95% yield) as a pale oil, which was used directly for next step. MS (ESI, pos. ion) m/z: 159.1 (M+1).

### Step 4:2-(2-(3-methyl-2-oxotetrahydropyrimidin-1(2H)-yl)ethyl)isoindoline-1,3-dione (I-361)

To a solution of 1-(2-((tert-butyldimethylsilyl)oxy)ethyl)-3-methyltetrahydropyrimidin-2(1H)-one, I-360 (600 mg, 3.8 mmol) in THF (20 mL) at 0-5° C. was added phthalimide (cas: 85-41-6, 592 mg, 1.05 equiv.), PPh₃ (1.5 g, 1.5 equiv) under N₂. After 30 minutes, DIAD (2.3 g, 3.0 equiv.) was added and the reaction mixture was allowed to warm to ambient temperature and stirred for 6 hours. The reaction was then quenched with water (5 mL), and extracted with DCM (3×20 mL). The combined organic extracts were washed with water (20 mL), brine (20 mL), and dried with Na₂SO₄, then concentrated under reduced pressure. The residue obtained was purified by column chromatography (50% EtOAc in Petroleum Ether) to afford phthalimide I-361 (280 mg 26% yield) as a yellow solid. MS (ESI, pos. ion) m/z: 288.1 (M+1).

### Step 5:1-(2-aminoethyl)-3-methyltetrahydropyrimidin-2(1H)-one (I-362)

To a solution of 2-(2-(3-methyl-2-oxotetrahydropyrimidin-1(2H)-yl)ethyl)isoindoline-1,3-dione, I-361 (280 mg, 0.98 mmol) in MeOH (5 mL) at 0-5° C. was added NH₂NH₂ (85% solution in H₂O, 200 mg, 5.6 equiv.). The reaction mixture was allowed to warm to ambient temperature and stirred for 12 hours. The slurry was then filtered, and the filtrate was concentrated to afford the crude amine I-362 (0.2 g) as a pale oil, which was taken forward to the next step. MS (ESI, pos. ion) m/z: 158.1 (M+1).

### Step 6: 2-((2-oxo-2-((2-(2-oxotetrahydropyrimidin-1(2H)-yl)ethyl)amino)ethyl)thio)acetic acid (I-363)

A solution of 1-(2-aminoethyl)-3-methyltetrahydropyrimidin-2(1H)-one, I-362 (200 mg, 1.27 mmol) and thiodiglycolic anhydride (cas: 3261-87-8, 300 mg, 1.8 equiv.) in 5 mL DCM was stirred for 2 hours, and then concentrated in vacuo to afford the crude product as a pale oil. Purification by preparative HPLC (Mobile Phase: MeCN/H₂O (with 0.1% Formic Acid) Gradient: 5-15% MeCN) afforded I-363 (50 mg, 14% yield) as a white solid. MS (ESI, pos. ion) m/z: 290.1 (M+1). ¹H NMR (400 MHz, CDCl₃) δ 7.47 (s, 1H), 3.59-3.56 (m, 2H), 3.5-3.42 (m, 2H), 3.36 (s, 2H), 3.36-3.31 (m, 2H), 3.31-3.26 (m, 2H), 3.20 (s, 2H), 2.94 (s, 3H), 2.01-1.96 (m, 2H).

### The Synthesis of I-366 Involved 3 Steps from Previously Described Intermediate I-357 as Depicted in the Following Scheme 124

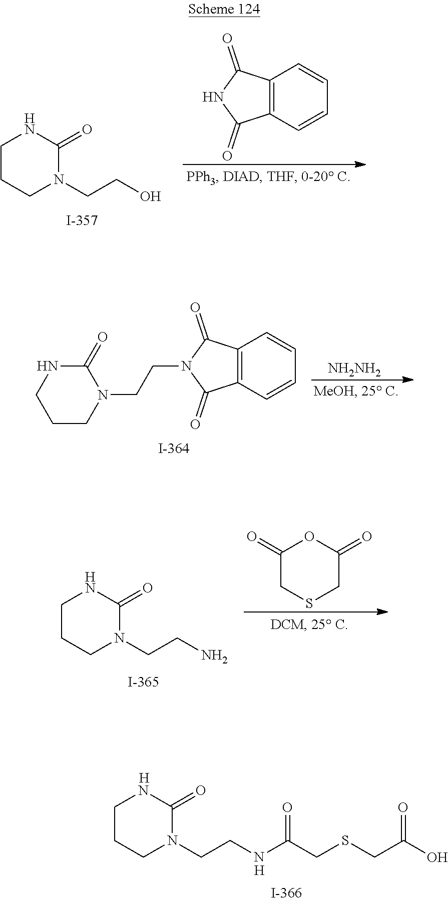

### 2-((2-oxo-2-((2-(2-oxotetrahydropyrimidin-1(2H)-yl)ethyl)amino)ethyl)thio) acetic acid (I-366)

### Step 1: 2-(2-(2-oxotetrahydropyrimidin-1(2H)-yl)ethyl)isoindoline-1,3-dione (I-364)

To a solution of 1-(2-hydroxyethyl)tetrahydropyrimidin-2(1H)-one, I-357 (1 g 6.9 mmol) in THF (30 mL) at 0-5° C. was added phthalimide (cas: 85-41-6, 1 g, 1.05 equiv.) and PPh₃ (2.7 g, 1.5 equiv.). After 30 minutes, DIAD (4.2 g, 3.0 equiv.) was added and the reaction mixture was allowed to warm to ambient temperature and stirred for 6 hours. The reaction was then quenched with water (5 mL), extracted with DCM (3×20 mL). The combined organic extracts were washed with water (20 mL), brine (20 mL), and dried with Na₂SO₄, then concentrated under reduced pressure. The residue obtained was purified by silica gel column chromatography (50% EtOAc in Petroleum Ether) to afford 800 mg (43% yield) of phthalimide I-364 as a yellow solid. MS (ESI, pos. ion) m/z: 274.1 (M+1).

### Step 2: 1-(2-aminoethyl)-3-methylpyrrolidin-2-one (I-365)

To a solution of 2-(2-(2-oxotetrahydropyrimidin-1(2H)-yl)ethyl)isoindoline-1,3-dione, I-364 (0.2 g, 0.7 mmol) in MeOH (5 mL) at 0-5° C. was added NH₂NH₂ (85% in H₂O, 200 mg, 5.5 mmol). The reaction mixture was allowed to warm to ambient temperature and stirred for 7 hours, then filtered, and the filtrate was concentrated to afford crude amine I-365 (0.12 g) as a pale oil. The crude product was used directly for the next step. MS (ESI, pos. ion) m/z: 145.1 (M+1).

### Step 3: 2-((2-oxo-2-((2-(2-oxotetrahydropyrimidin-1(2H)-yl)ethyl)amino)ethyl)thio)acetic acid (I-366)

A solution of 1-(2-aminoethyl)-3-methylpyrrolidin-2-one, I-365 (120 mg, 0.83 mmol) and thiodiglycolic anhydride (cas: 3261-87-8, 167 mg, 1.5 equiv.) in 5 mL of DCM was stirred for 2 hours, and then concentrated in vacuo to give the crude product as a pale oil. The crude product was purified by preparative HPLC (Mobile Phase: MeCN/H₂O (0.1% TFA), gradient: 5-15% MeCN) to afford 80 mg (35% yield) of I-366 as a white solid. MS (ESI, pos. ion) m/z: 276.2 (M+1). ¹H NMR (400 MHz, CDCl₃) δ 7.54 (s, 1H), 6.71 (s, 1H), 3.59-3.52 (m, 2H), 3.51 (d, J=5.1 Hz, 2H), 3.42-3.36 (m, 3H), 3.34-3.29 (m, 3H), 1.99 (s, 2H), 1.25 (s, 2H).

### The Synthesis of Compound I-373 Involved 8 Steps as Depicted in the Following Scheme 125

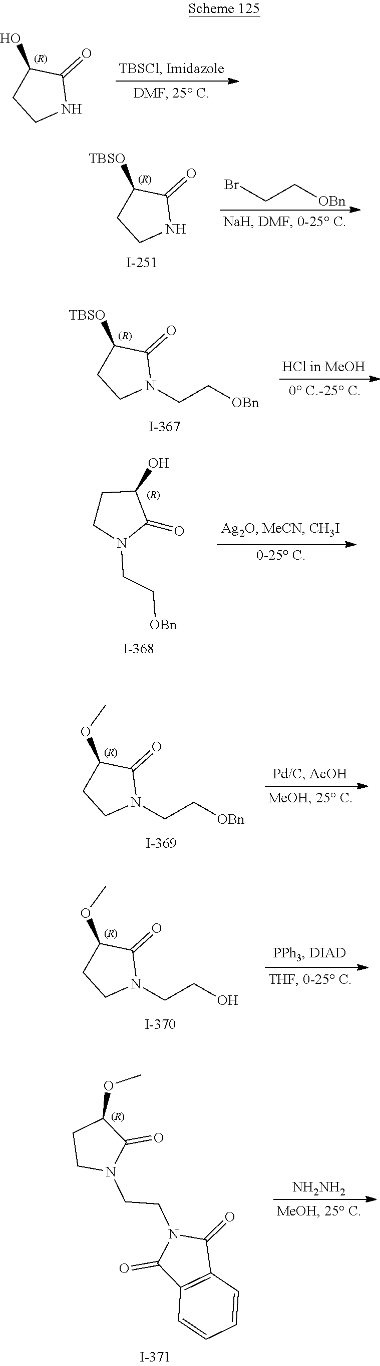

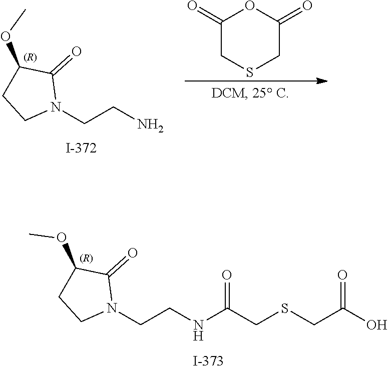

The chemical structure of the target compound was confirmed by 1HNMR and LC-MS with the purity >95% (HPLC 214 & 254 nm).

### Step 1: (R)-1-(2-(benzyloxy)ethyl)-3-((tert-butyldimethylsilyl)oxy)pyrrolidin-2-one (I-367)

To a solution of (R)-3-((tert-butyldimethylsilyl)oxy)pyrrolidin-2-one, I-251 (8 g, 37.2 mmol) in anhydrous DMF (80 mL) at 0-5° C. was added NaH (60% dispersion in mineral oil, 3.72 g, 2.5 equiv.) under N₂. The reaction mixture was stirred for 30 minutes at at 0-5° C. and ((2-bromoethoxy)methyl)benzene (cas: 1462-37-9, 11.9 g, 1.5 equiv.) was added. The reaction mixture was allowed to warm to ambient temperature. After 12 hours, LC-MS indicated that the reaction was complete. H₂O (20 mL) was added and the reaction mixture was extracted with EtOAc (20 mL×3). The combined organic extracts were concentrated under reduced pressure and the crude product was purified by silica gel chromatography (Petroleum ether:EtOAc=1:1) to provide I-367 (5 g, 39% yield) as a pale oil. MS (ESI, pos. ion) m/z: 350 (M+1).

### Step 2: (R)-1-(2-(benzyloxy)ethyl)-3-hydroxypyrrolidin-2-one (1-368)

To a solution of (R)-1-(2-(benzyloxy)ethyl)-3-((tert-butyldimethylsilyl)oxy)pyrrolidin-2-one, I-367 (5 g, 14.32 mmol) was added HCl in MeOH (30% v/v, 30 mL) and the reaction mixture was stirred at 0-5° C. for 0.5 hours. Once LC-MS analysis indicated reaction completion, the reaction mixture was concentrated and the crude product was purified by silica gel chromatography (Petroleum ether:EtOAc=1:10) to afford I-368 (2.36 g, 70% yield) as a pale oil. MS (ESI, pos. ion) m/z: 236 (M+1).

### Step 3: (R)-1-(2-(benzyloxy)ethyl)-3-methoxypyrrolidin-2-one (I-369)

To a solution of (R)-1-(2-(benzyloxy)ethyl)-3-hydroxypyrrolidin-2-one, I-368 (2.36 g, 10.0 mmol) in anhydrous MeCN (30 ml) at 0-5° C. was added Ag₂O (4.65 g, 2.0 equiv.) under N₂. The reaction mixture was then stirred for 5 minutes and CH₃I (1.28 g 0.9 equiv.) was added. The reaction mixture was was allowed to warm to ambient temperature and stirred for 10 hours. When LC-MS analysis indicated that the reaction was complete, the reaction mixture was filtered and the filtrate was concentrated under reduced pressure to afford the crude product, which was purified by silica gel chromatography (Petroleum ether:EtOAc=1:1) to provide I-369 (1.7 g, 68%) as a pale oil. MS (ESI, pos. ion) m/z: 250 (M+1).

### Step 4: (R)-1-(2-hydroxyethyl)-3-methoxypyrrolidin-2-one (1-370)

To a solution of (R)-1-(2-(benzyloxy)ethyl)-3-methoxypyrrolidin-2-one, I-369 (1.7 g, 6.82 mmol) in MeOH (10 mL) was added Pd/C (170 mg) quickly under H₂ (1 atm). The reaction mixture was then stirred at ambient temperature for 48 hours. When LC-MS analysis indicated that the reaction was complete, the reaction mixture was filtered, and the filtrate was concentrated under reduced pressure to a residue. The crude product was purified by silica gel chromatography (Petroleum ether:EtOAc=1:10) to provide I-370 (0.15 g, 14% yield) as a pale oil. MS (ESI, pos. ion) m/z: 160 (M+1).

### Step 5: (R)-2-(2-(3-methoxy-2-oxopyrrolidin-1-yl)ethyl)isoindoline-1,3-dione (I-371)

A solution of (R)-1-(2-hydroxyethyl)-3-methoxypyrrolidin-2-one, 1-370 (0.15 g, 0.94 mmol), phthalimide (cas: 85-41-6, 0.148 g, 1.1 equiv.) and PPh₃ (0.369 g, 1.5 equiv.) in anhydrous THF (3 mL) was stirred at 0-5° C. for 0.5 hours. Then DIAD (0.569 g, 3.0 equiv.) was added and the reaction mixture was allowed to warm to ambient temperature and stirred for 7 hours. The reaction mixture was then concentrated to afford the crude product, which was purified by silica gel chromatography (Petroleum ether:EtOAc=1:10) to provide I-371 (0.5 g, 50% purity, 50% yield) as a pale oil. MS (ESI, pos. ion) m/z: 275 (M+1).

### Step 6: (R)-1-(2-aminoethyl)-3-methoxypyrrolidin-2-one (I-372)

To a solution of (R)-2-(2-(3-methoxy-2-oxopyrrolidin-1-yl)ethyl)isoindoline-1,3-dione, I-371 (0.5 g, 1.74 mmol) in MeOH (5 mL) was added hydrazine hydrate (80% solution, 0.130 g, 1.5 equiv.). The reaction mixture was stirred for 12 hours at ambient temperature, then filtered. The filtrate was concentrated and the crude product, I-372 (0.5 g) was used directly in the next step. MS (ESI, pos. ion) m/z: 159 (M+1).

### Step 7: (R)-2-((2-((2-(3-methoxy-2-oxopyrrolidin-1-yl)ethyl)amino)-2-oxoethyl)thio) acetic acid (I-373)

To a solution of (R)-1-(2-aminoethyl)-3-methoxypyrrolidin-2-one, I-372 (0.5 g, 3.3 mmol) and thiodiglycolic anhydride (cas: 3261-87-8, 0.653 g, 1.5 equiv.) in 5 mL of DCM was added TEA (0.066 g, 0.2 equiv.). The reaction mixture was stirred for 2 hours, and then concentrated under reduced pressure to give the crude product, which was purified by preparative HPLC (Mobile Phase: MeCN/H₂O (0.1% TFA), Gradient: 5-15% MeCN) to afford I-373 (50 mg, 5% yield) as a pale oil. MS (ESI, pos. ion) m/z: 291.1 (M+1). ¹HNMR (400 MHz, DMSO) δ 12.68 (s, 1H), 8.10 (s, 1H), 3.88 (s, 1H), 3.38 (s, 3H), 3.35 (s, 2H), 3.33-3.26 (m, 3H), 3.19 (m, 5H), 2.32-2.20 (m, 1H), 1.76 (m, 1H).

### The Synthesis of Compound I-380 Involved 8 Steps as Depicted in the Following Scheme 126

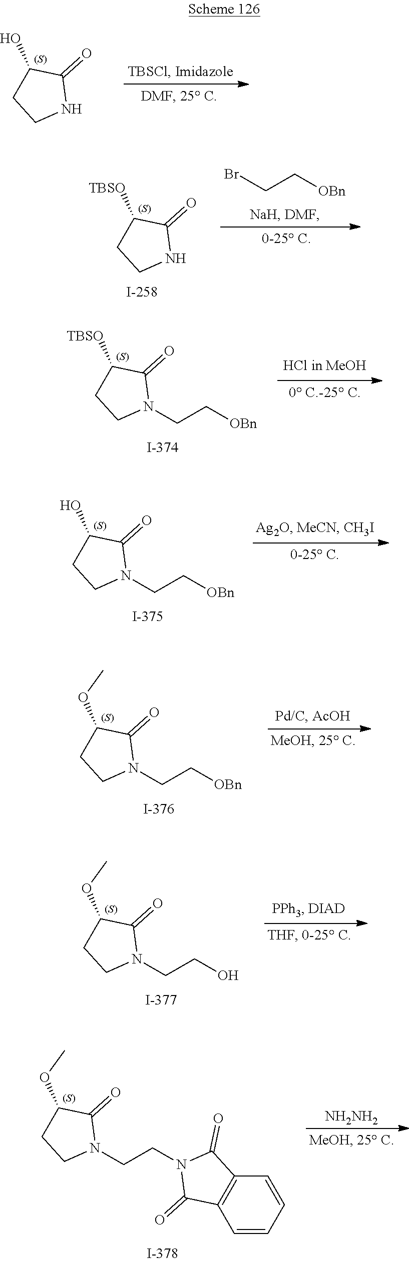

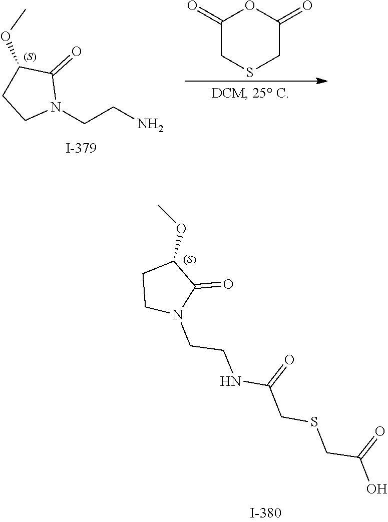

### (S)-2-((2-((2-(3-methoxy-2-oxopyrrolidin-1-yl)ethyl)amino)-2-oxoethyl)thio) acetic acid (I-380)

The synthetic route for I-380 is similar to that of I-373 and commenced with (S)-3-hydroxypyrrolidin-2-one (cas: 34368-52-0).Carboxylic acid I-380 (pale oil, 50 mg, 3% yield) was isolated by preparative HPLC (Mobile Phase: MeCN/H₂O (with 0.1% TFA), Gradient: 5-15% MeCN). MS (ESI, pos. ion) m/z: 291.1 (M+1). ¹H NMR (400 MHz, DMSO) δ 8.11 (s, 1H), 3.89 (m, 1H), 3.38 (s, 3H), 3.35 (s, 2H), 3.33-3.25 (m, 3H), 3.25-3.14 (m, 5H), 2.31-2.21 (m, 1H), 1.82-1.70 (m, 1H).

### The Synthesis of I-386 Involved 6 Steps from Previously Described Intermediate I-259 as Depicted in the Following Scheme 127

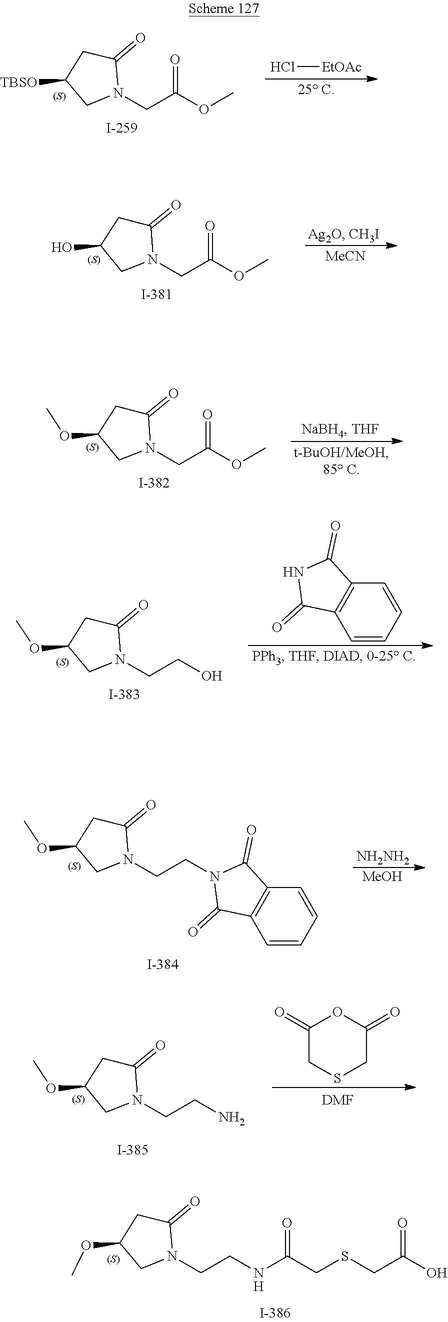

### Step 1: Methyl (S)-2-(4-hydroxy-2-oxopyrrolidin-1-yl)acetate (I-381)

To a solution of methyl (S)-2-(4-((tert-butyldimethylsilyl)oxy)-2-oxopyrrolidin-1-yl)acetate, I-259 (2.1 g, 7.32 mmol) in MeOH (15 mL) at 0-5° C. was added HCl in EtOAc (2M, 15 mL) under N₂. The reaction mixture was allowed to warm to ambient temperature and stirred for 4 hours. After reaction completion, ammonium hydroxide (ca. 0.2 mL) was added to adjust the pH to 7, and then the reaction mixture was concentrated under reduced pressure. The residue obtained was purified by silica gel chromatography to afford ester I-381 (1.3 g, 102% yield) as a pale oil. MS (ESI, pos. ion) m/z: 174.1 (M+1).

### Step 2: Methyl (S)-2-(4-methoxy-2-oxopyrrolidin-1-yl)acetate (I-382)

To a solution of methyl(S)-2-(4-hydroxy-2-oxopyrrolidin-1-yl)acetate, I-381 (1.3 g, 7.51 mmol) in acetonitrile (15 ml) at 0-5° C. was added silver oxide (cas: 260667-12-3, 2.62 g, 1.5 equiv.) and iodomethane (0.96 g, 0.9 equiv.) under N₂. The reaction mixture was allowed to warm to ambient temperature and stirred for 12 hours. Upon completion of the reaction, the reaction mixture was filtered through celite. The filtrate was concentrated under reduced pressure and the residue obtained was purified by silica gel chromatography to afford ester I-382 (0.48 g, 35% yield) as a pale oil. MS (ESI, pos. ion) m/z: 184.1 (M+1).

### Step 3: (S)-1-(2-hydroxyethyl)-4-methoxypyrrolidin-2-one (1-383)

To a solution of methyl(S)-2-(4-methoxy-2-oxopyrrolidin-1-yl)acetate, I-382 (0.25 g, 1.50 mmol) in t-BuOH (10 mL) was added sodium borohydride (0.228 g, 4.0 equiv.) in MeOH (0.5 mL) under N₂. The reaction mixture was stirred for 0.5 hours at 85° C. Upon completion of the reaction, the reaction mixture was concentrated under reduced pressure. The residue afforded was quenched with water (2 mL), and extracted with DCM (3×20 mL). The combined organic extracts were then washed with water (20 mL), brine (20 mL), and dried with Na₂SO₄, then concentrated and the residue obtained was purified by silica gel chromatography to afford alcohol I-383 (0.11 g, 46% yield) as a pale oil. MS (ESI, pos. ion) m/z: 160.1 (M+1).

### Step 4: (S)-2-(2-(4-methoxy-2-oxopyrrolidin-1-yl)ethyl)isoindoline-1,3-dione (I-384)

To a solution of (S)-1-(2-hydroxyethyl)-4-methoxypyrrolidin-2-one, I-383 (0.31 g 1.95 mmol) in THF (5 mL) was added phthalimide (0.32 g, 1.1 equiv.), and PPh₃ (0.77 g 1.5 equiv.). The reaction mixture was stirred for 0.5 h at 0-5° C. under N₂ and DIAD (1.18 g, 3.0 equiv.) was added. The reaction mixture was allowed to warm to ambient temperature and stirred at ambient temperature for 12 hours, then concentrated under reduced pressure. The residue obtained was purified by silica gel chromatography to afford phthalimide I-384 as a yellow oil (0.40 g, 71% yield). MS (ESI, pos. ion) m/z: 289.1 (M+1).

### Step 5: (S)-1-(2-aminoethyl)-4-methoxypyrrolidin-2-one (I-385)

To a solution of (S)-2-(2-(4-methoxy-2-oxopyrrolidin-1-yl)ethyl)isoindoline-1,3-dione, I-384 (0.40 g, 1.39 mmol) in MeOH (10 mL) at 0-5° C. was added hydrazine (85% in H₂O, 0.06 g, 1.2 equiv.). The reaction mixture was allowed to warm to ambient temperature and stirred for 12 hours, then concentrated under reduced pressure. DCM (10 ml) was added to the residue and the slurry was filtered. The filtrate was concentrated to afford the crude amine I-385 as a pale oil, 0.18 g, 82% yield), which was used directly in the next step. MS (ESI, pos. ion) m/z: 159.1 (M+1).

### Step 6: (S)-2-((2-((2-(4-methoxy-2-oxopyrrolidin-1-yl)ethyl)amino)-2-oxoethyl)thio) acetic acid (I-386)

A solution of (S)-1-(2-aminoethyl)-4-methoxypyrrolidin-2-one, I-385 (0.18 g, 1.13 mmol) and thiodiglycolic anhydride (0.19 g, 1.3 equiv.) in DMF (5 mL) was stirred for 2 hours at 25° C., then concentrated under reduced pressure to give the crude product as a pale oil. The crude product was purified by preparative HPLC (Mobile Phase: MeCN/H₂O (0.1% TFA), Gradient: 1-3% MeCN) to afford 137 mg (42% yield) of I-386 as a pale oil. MS (ESI, pos. ion) m/z: 291.1 (M+1). ¹H NMR (400 MHz, CDCl₃) δ 7.56 (s, 1H), 4.06 (t, 1H), 3.82 (dd, 1H), 3.68-3.59 (m, 2H), 3.46-3.36 (m, 5H), 3.32 (s, 3H), 3.27 (s, 2H), 2.72 (dd, 6.7 Hz, 1H), 2.53 (dd, 1.6 Hz, 1H).

The synthesis of I-392 involved 6 steps from previously described intermediate I-252 as depicted in the following Scheme 128.

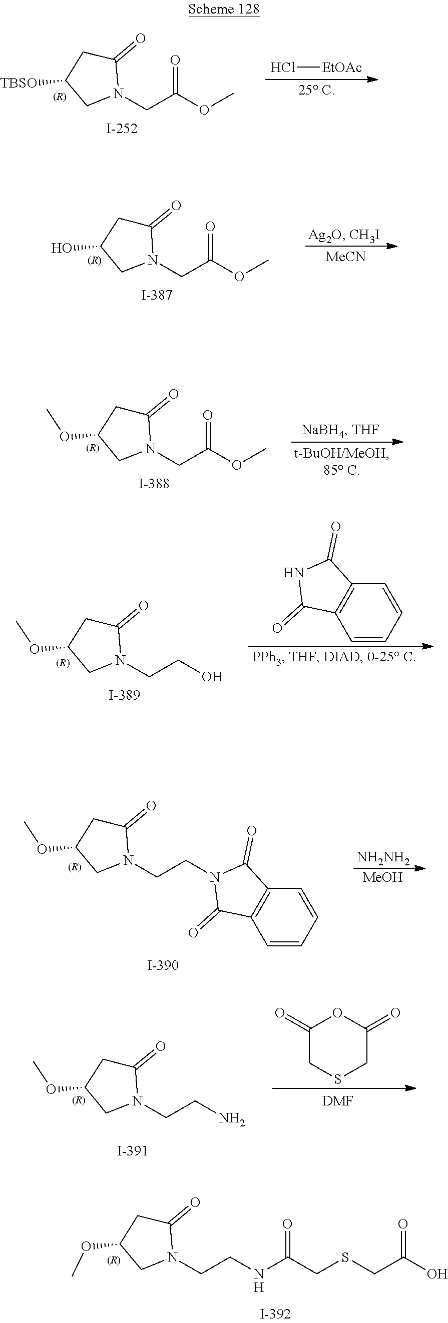

### Step 1: Methyl (R)-2-(4-hydroxy-2-oxopyrrolidin-1-yl)acetate (I-387)

To a solution of methyl (R)-2-(4-((tert-butyldimethylsilyl)oxy)-2-oxopyrrolidin-1-yl)acetate, I-252 (3.7 g, 12.89 mmol) in MeOH (20 mL) at 0-5° C. was added HCl in EtOAc (2M, 20 mL) under N₂. The reaction mixture was stirred for 4 hours. Upon completion of the reaction, ammonium hydroxide (about 0.4 ml) was added to adjust pH 7, and then the reaction mixture was concentrated under reduced pressure. The residue obtained was purified by silica gel chromatography to afford alcohol I-387 as a pale oil (2.1 g, 94% yield). MS (ESI, pos. ion) m/z: 174.1 (M+1).

### Step 2: Methyl (R)-2-(4-methoxy-2-oxopyrrolidin-1-yl)acetate (I-388)

To a solution of methyl (R)-2-(4-hydroxy-2-oxopyrrolidin-1-yl)acetate, I-387 (2.1 g, 12.14 mmol) in acetonitrile (20 ml) at 0-5° C. was added silver oxide (4.22 g, 1.5 equiv.) and iodomethane (1.55 g, 0.9 equiv.) under N₂. The reaction mixture was allowed to warm to ambient temperature and stirred for 12 hours, then filtered through celite. The filtrate was concentrated under reduced pressure. The residue obtained was purified by silica gel chromatography to afford ester I-388 as a pale oil (0.83 g, 37% yield). MS (ESI, pos. ion) m/z: 184.1 (M+1).

### Step 3: (R)-1-(2-hydroxyethyl)-4-methoxypyrrolidin-2-one (I-389)

To a solution of methyl (R)-2-(4-methoxy-2-oxopyrrolidin-1-yl)acetate, 1-388 (1.2 g, 6.42 mmol) in t-BuOH (10 mL) was added sodium borohydride (0.73 g, 3.0 equiv.) under N₂. The reaction mixture was stirred for 0.5 hours at 80° C., then cooled and concentrated under reduced pressure. The residue was quenched with water (2 mL) and extracted with DCM (3×20 mL). The combined organic extracts were then washed with water (20 mL), brine (20 mL), and dried with Na₂SO₄, then concentrated and the residue obtained was purified by silica gel chromatography to afford alcohol I-389 as a pale oil (0.52 g, 51% yield). MS (ESI, pos. ion) m/z: 160.1 (M+1).

### Step 4: (R)-2-(2-(4-methoxy-2-oxopyrrolidin-1-yl)ethyl)isoindoline-1,3-dione (I-390)

To a solution of (R)-1-(2-hydroxyethyl)-4-methoxypyrrolidin-2-one, I-389 (0.58 g, 3.64 mmol) in THF (6 mL) was added phthalimide (0.59 g, 1.1 equiv.) and PPh₃ (1.43 g, 1.5 equiv.). The reaction mixture was stirred for 0.5 hours in an ice-water bath under N₂. Then DIAD (2.21 g, 3.0 equiv.) was added and the reaction mixture was allowed to warm to ambient temperature and stirred for 12 hours, then concentrated under reduced pressure. The residue obtained was purified by silica gel chromatography to afford phthalimide I-390 as a yellow oil (0.45 g, 43% yield). MS (ESI, pos. ion) m/z: 289.1 (M+1).

### Step 5: (R)-1-(2-aminoethyl)-4-methoxypyrrolidin-2-one (I-391)

To a solution of (S)-2-(2-(4-methoxy-2-oxopyrrolidin-1-yl)ethyl)isoindoline-1,3-dione, I-390 (0.45 g, 1.56 mmol) in MeOH (10 mL) at 0-5° C. was added hydrazine (85% in H₂O, 0.07 g, 1.87 mmol, 1.2 equiv.). The reaction mixture was allowed to warm to ambient temperature and stirred for 12 hours, then concentrated. The residue was diluted in DCM (10 mL) and the slurry was filtered. The filtrate was concentrated to afford the crude amine I-391 as a pale oil (0.21 g, 85% yield, 85% purity), which was used directly in the next step. MS (ESI, pos. ion) m/z: 159.1 (M+1).

### Step 6: (R)-2-((2-((2-(4-methoxy-2-oxopyrrolidin-1-yl)ethyl)amino)-2-oxoethyl)thio) acetic acid (I-392)

A solution of (R)-1-(2-aminoethyl)-4-methoxypyrrolidin-2-one, I-391 (0.21 g, 1.33 mmol) and thiodiglycolic anhydride (0.23 g, 1.3 equiv.) in DMF (5 mL) was stirred for 2 hours at 25° C., then the reaction mixture was concentrated under reduced pressure and the crude product was purified by preparative HPLC (Mobile Phase: MeCN/H₂O (0.1% TFA), Gradient: 1-3% MeCN) to give 117.5 mg (41% yield) of acid I-392 as a pale oil. MS (ESI, pos. ion) m/z: 291.1 (M+1). ¹H NMR (400 MHz, CDCl₃) δ 7.52 (s, 1H), 4.04 (t, 1H), 3.78 (dd, 1H), 3.63-3.55 (m, 2H), 3.42-3.32 (m, 5H), 3.30 (s, 3H), 3.27 (s, 2H), 2.67 (dd, 1H), 2.47 (dd, 1H).

### The Synthesis of Compounds (S*)-I-395 and (R*)-I-395 Involved 4 Steps as Depicted in Scheme 129

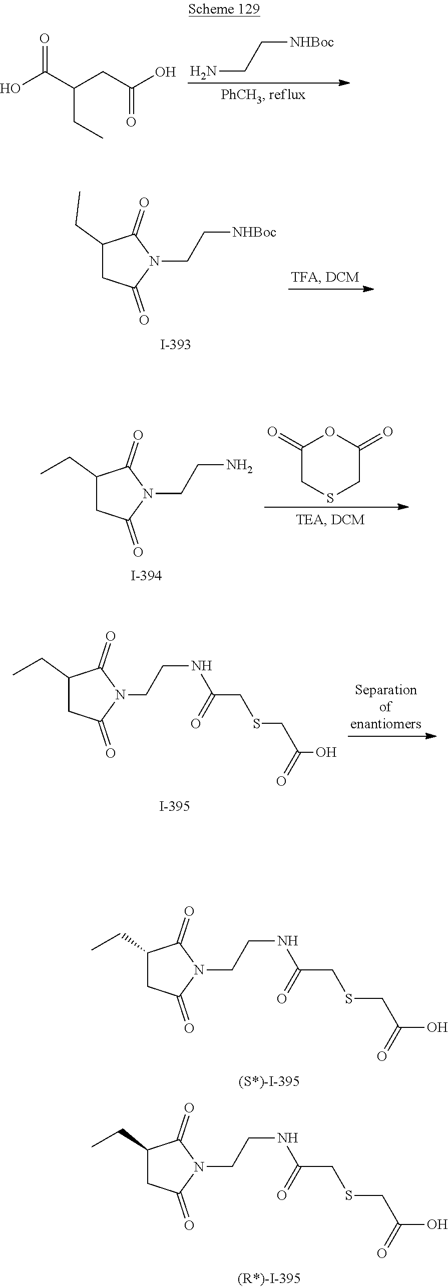

The chemical structure of the target compound was confirmed by ¹H NMR and LC-MS with the purity >95% (HPLC 214 & 254 nm).

### Step 1: Tert-butyl (2-(3-ethyl-2,5-dioxopyrrolidin-1-yl)ethyl)carbamate (I-393)

A solution of 2-ethylsuccinic acid (cas: 636-48-6, 0.2 g, 1.37 mmol) and tert-butyl (2-aminoethyl)carbamate (0.26 g, 1.2 equiv.) in 5 mL of PhCH₃ was heated to reflux for 7 hours. After LC-MS analysis indicated that the reaction was complete, the reaction mixture was concentrated under reduced pressure to give the crude product, which was purified by preparative TLC (EtOAc: Petroleum ether=2:1) to afford imide I-393 (0.15 g, 41% yield) a pale oil. MS (ESI, pos. ion) m/z: 293 (M+Na).

### Step 2:1-(2-aminoethyl)-3-ethylpyrrolidine-2,5-dione (I-394)

A solution of tert-butyl (2-(3-ethyl-2,5-dioxopyrrolidin-1-yl)ethyl)carbamate, I-393 (250 mg, 0.93 mmol) in TFA/DCM (1:3 v/v, 8 mL) was stirred at 25° C. for 1 hour. When LC-MS analysis indicated that the reaction was complete, the reaction mixture was concentrated under reduced pressure to give 1-(2-aminoethyl)-3-ethylpyrrolidine-2,5-dione, 1-394 (500 mg) as a pale oil, which was used directly for next step. MS (ESI, pos. ion) m/z: 171 (M+1).

### Step 3: 2-((2-((2-(3-ethyl-2,5-dioxopyrrolidin-1-yl)ethyl)amino)-2-oxoethyl)thio) acetic acid (I-395)

To a solution of (S)-1-(2-aminoethyl)-3-hydroxypyrrolidine-2, 5-dione, I-394 (500 mg, 1.77 mmol) and thiodiglycolic anhydride (257 mg, 1.1 equiv.) in 5 mL DCM was added TEA (257 mg, 1.1 equiv.). The reaction mixture was stirred for 2 hours and was concentrated in vacuo to give the crude product. The crude product was purified by preparative HPLC (Mobile Phase: MeCN/H₂O (0.1% formic acid)) to afford the racemic carboxylic acid I-395 as a pale oil (220 mg, 41% yield). MS (ESI, pos. ion) m/z: 303.1 (M+1).

### (S)-2-((2-((2-(3-ethyl-2,5-dioxopyrrolidin-1-yl)ethyl)amino)-2-oxoethyl)thio) acetic acid ((S*)-I-395)

(S*)-1-395 (pale oil, 80 mg, 36% yield) was isolated by chiral separation (Superchiral S-AD (Chiralway), 2 cm I.D.*25 cm Length, 5 um, CO₂/EtOH=70/30 (v/v)) from racemic acid I-395. MS (ESI, pos. ion) m/z: 303.1 (M+1). ¹H NMR (400 MHz, CDCl₃) δ 7.22 (s, 1H), 3.74-3.61 (m, 2H), 3.58-3.45 (m, 2H), 3.33 (m, 4H), 2.93-2.72 (m, 2H), 2.40 (m, 1H), 1.99-1.86 (m, 1H), 1.65-1.53 (m, 1H), 0.99 (m, 3H).

### (R)-2-((2-((2-(3-ethyl-2,5-dioxopyrrolidin-1-yl)ethyl)amino)-2-oxoethyl)thio) acetic acid ((R*)-I-395)

(R*)-I-395 (pale oil, 80 mg, 36% yield) is isolated by chiral separation (Superchiral S-AD (Chiralway), 2 cm I.D.*25 cm Length, 5 μm, CO₂/EtOH=70/30 (v/v)) from racemic acid I-395. MS (ESI, pos. ion) m/z: 303.1 (M+1). ¹H NMR (400 MHz, CDCl₃) δ 7.48 (s, 1H), 3.66 (m, 2H), 3.47 (m, 2H), 3.31 (s, 4H), 2.94-2.67 (m, 2H), 2.37 (m, 1H), 1.88 (m, 1H), 1.56 (m, 1H), 0.95 (m 3H).

### The Synthesis of Compound I-398 Involved 3 Steps as Depicted in Scheme 130. I-399, the Enantiomer of I-398, was Made Using a Similar Route

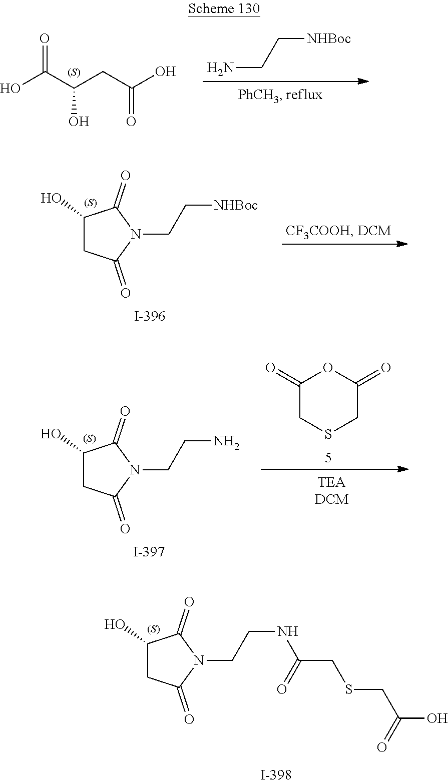

### Step 1: Tert-butyl (S)-(2-(3-hydroxy-2,5-dioxopyrrolidin-1-yl)ethyl)carbamate (I-396)

A solution of (S)-2-hydroxysuccinic acid (cas: 97-67-6, 2 g, 14.9 mmol) and tert-butyl (2-aminoethyl)carbamate (2.88 g, 1.2 equiv.) in 10 mL of toluene was heated to reflux for 7 hours. When LC-MS analysis indicated that the reaction was complete, the reaction mixture was concentrated under reduced pressure to give the crude product, which was purified by silica gel chromatography (EtOAc: Petroleum ether=2:1) to afford 1.9 g (49% yield) of intermediate tert-butyl(S)-(2-(3-hydroxy-2,5-dioxopyrrolidin-1-yl)ethyl)carbamate I-396 as a pale oil. MS (ESI, pos. ion) m/z: 281 (M+Na).

### Step 2: (S)-1-(2-aminoethyl)-3-hydroxypyrrolidine-2, 5-dione (I-397)

A solution of tert-butyl(S)-(2-(3-hydroxy-2,5-dioxopyrrolidin-1-yl)ethyl)carbamate, I-396 (300 mg, 1.06 mmol) in TFA/DCM (1:3 v/v, 8 mL) was stirred at 25° C. for 1 hour. When LC-MS analysis indicated that the reaction was complete, the reaction mixture was concentrated under reduced pressure to afford(S)-1-(2-aminoethyl)-3-hydroxypyrrolidine-2, 5-dione, I-397 (400 mg) as a pale oil, which was used directly for next step. MS (ESI, pos. ion) m/z: 159 (M+1).

### Step 3: (S)-2-((2-((2-(3-hydroxy-2,5-dioxopyrrolidin-1-yl)ethyl)amino)-2-oxoethyl)thio) acetic acid (I-398)

A solution of (S)-1-(2-aminoethyl)-3-hydroxypyrrolidine-2, 5-dione, I-397 (200 mg, 1.3 mmol) and thiodiglycolic anhydride (167 mg, 1.3 mmol) in 5 mL of DCM was added TEA (157 mg, 1.2 equiv.). The reaction mixture was stirred for 2 hours, then concentrated under reduced pressure to give the crude product. The crude product was purified by preparative HPLC to give the carboxylic acid I-398 (83 mg, 22% yield) as a pale oil. MS (ESI, pos. ion) m/z: 291.2 (M+1). ¹H NMR (400 MHz, DMSO-d₆): δ 8.16-8.13 (m, 1H), 4.48-4.45 (m, 1H), 3.45-3.42 (m, 2H), 3.35 (s, 2H), 3.26-3.17 (m, 2H), 3.14 (s, 2H), 2.98-2.91 (m, 1H), 2.55 (s, 1H), 2.44-2.39 (m, 1H).

### R-2-((2-((2-(3-hydroxy-2,5-dioxopyrrolidin-1-yl)ethyl)amino)-2-oxoethyl)thio) acetic acid (I-399)

I-399 was prepared from (2R)-malic acid as a pale oil in 79% yield. MS (ESI, pos. ion) m/z: 291.2 (M+1). ¹H NMR (400 MHz, DMSO-d₆): δ 8.15-8.12 (m, 1H), 4.47-4.44 (m, 1H), 3.45-3.42 (m, 2H), 3.34 (s, 2H), 3.26-3.16 (m, 2H), 3.14 (s, 2H), 2.97-2.91 (m, 1H), 2.54 (s, 1H), 2.44-2.36 (m, 1H).

### The Synthesis of Racemic Compound (±)-I-403 Involved 3 Steps as Depicted in the Following Scheme 131. The Intermediate I-404 was then Isolated by Chiral Separation

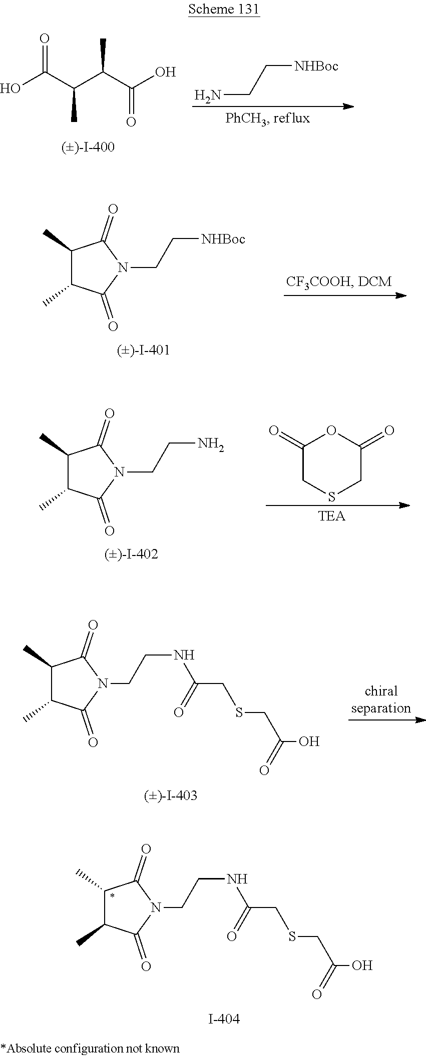

### 2-((2-((2-(3,4-dimethyl-2,5-dioxopyrrolidin-1-yl)ethyl)amino)-2-oxoethyl)thio) acetic acid (I-404)

The synthetic route for I-404 was similar to that of I-398 and commenced with (+)-(2R*,3R*)-2,3-dimethylsuccinic acid, (+) I-400 (cas: 13545-04-5). I-404 (pale oil, 30 mg, 15% yield) was isolated by chiral separation (Superchiral S-AD (Chiralway), 2 cm I.D.*25 cm Length, 5 μm, Hexane/EtOH=60/40 (v/v)) from racemic acid I-403. MS (ESI, pos. ion) m/z: 303.1 (M+1). ¹H NMR (400 MHz, CDCl₃): δ 7.34 (s, 1H), 5.6 (s, 1H), 3.67 (m, 2H), 3.5 (s, 2H), 3.31 (m, 4H), 2.4 (m, 2H), 1.3 (m, 6H).

### The Synthesis of (R*)-I-409 and (S*)-I-409 Involved 5 Steps as Depicted in the Following Scheme 132

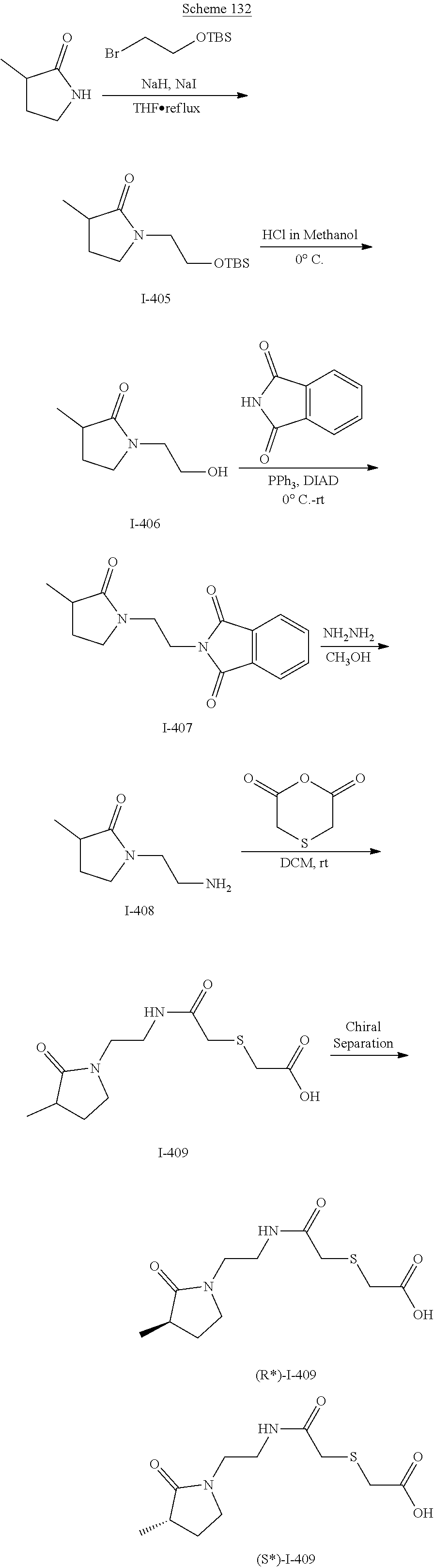

### Step 1: 1-(2-((tert-butyldimethylsilyl)oxy)ethyl)-3-methylpyrrolidin-2-one (I-405)

To a solution of 3-methylpyrrolidin-2-one (cas: 2555 May 7, 0.3 g, 3 mmol) in THF (10 mL) at 0-5° C. was added NaH (0.242 g, 2 equiv.) under N₂. The reaction mixture was stirred for 0.5 hours, then (2-bromoethoxy)(tert-butyl)dimethylsilane (cas: 86864-60-0, 0.86 g, 1.2 equiv.) was added. The reaction mixture was allowed to warm to ambient temperature and stirred for 5 hours. The reaction was then quenched with water (5 mL), and extracted with DCM (3×20 mL). The combined organic extracts were then washed with water (20 mL), brine (20 mL), dried with Na₂SO₄ and concentrated under reduced pressure. The residue obtained was purified by silica gel chromatography to afford I-405 (0.25 g, 32% yield) as a pale oil. MS (ESI, pos. ion) m/z: 258 (M+1).

### Step 2:1-(2-Hydroxy-ethyl)-3-methyl-pyrrolidin-2-one (I-406)

To a solution of 1-(2-((tert-butyldimethylsilyl)oxy)ethyl)-3-methylpyrrolidin-2-one, I-405 (0.3 g, 3 mmol) in MeOH (10 mL) at 0-5° C. was added HCl in MeOH (30%, 3 mL) under N₂. The mixture was stirred for 0.5 hours. The reaction mixture was then concentrated under reduced pressure to afford the crude alcohol I-406 as a pale oil (0.25 g), which was used directly in the next step. MS (ESI, pos. ion) m/z: 144 (M+1).

### Step 3:2-(2-(3-Methyl-2-oxopyrrolidin-1-yl)ethyl)isoindoline-1,3-dione (I-407)

To a solution of 1-(2-hydroxyethyl)-3-methylpyrrolidin-2-one, I-406 (0.25 g, 1.75 mmol) in THF (5 mL) was added phthalimide (cas: 85-41-6, 0.25 g, 1.5 equiv.) and PPh₃ (0.68 g, 1.5 equiv.). The reaction mixture was cooled to 0-5° C. and DIAD (1.059 g, 3.0 equiv.) was added. The reaction mixture was allowed to warm to ambient temperature and stirred for 6 hours, then quenched with water (5 mL), and extracted with DCM (3×20 mL). The combined organic extracts were then washed with water (20 mL), brine (20 mL), dried with Na₂SO₄ and concentrated under reduced pressure to afford the crude product, I-407 (purity: 67%, 0.3 g, 48% yield), which was taken to the next step without further purification. MS (ESI, pos. ion) m/z: 273 (M+1).

### Step 4:1-(2-aminoethyl)-3-methylpyrrolidin-2-one (I-408)

To a solution of 2-(2-(3-methyl-2-oxopyrrolidin-1-yl)ethyl)isoindoline-1,3-dione, I-407 (0.3 g, 3 mmol) in MeOH (10 mL) at 0-5° C. was added NH₂NH₂ (85% in H₂O, 1.03 mL, 1.2 equiv.). The reaction mixture was allowed to warm to ambient temperature, stirred for 7 hours, and then filtered. The filtrate was concentrated to afford a pale oil. The crude product, I-408 (0.35 g) was used directly in the next step. MS (ESI, pos. ion) m/z: 143 (M+1).

### Step 5: 2-((2-((2-(3-methyl-2-oxopyrrolidin-1-yl)ethyl)amino)-2-oxoethyl)thio) acetic acid (I-409)

To a solution of 1-(2-aminoethyl)-3-methylpyrrolidin-2-one, I-408 (350 mg, 1.3 mmol) and thiodiglycolic anhydride (167 mg, 1.0 equiv.) in 5 mL of DCM was added TEA (157 mg, 1.56 mmol). The reaction mixture was stirred for 2 hours and was concentrated in vacuo to give the crude acid, which was purified by preparative HPLC to afford racemic acid I-409. (R*)-I-409 (30 mg, 5%, absolute configuration not known) was isolated by chiral separation (CHIRALPAK AD-H column, Mobile phase: Hexane/EtOH/AcOH=80/20/0.1 (v/v/v)) from racemic I-409. MS (ESI, pos. ion) m/z: 275.2 (M+1). ¹H NMR (400 MHz, CDCl₃): δ 7.38 (s, 1H), 3.5 (m, 6H), 3.4 (s, 2H), 3.2 (s, 2H), 2.6 (m, 1H), 2.3 (m, 1H), 1.6 (m, 1H), 1.1 (m, 3H).

### 2-((2-((2-(3-methyl-2-oxopyrrolidin-1-yl)ethyl)amino)-2-oxoethyl)thio) acetic acid (S*)-I-409)

Isolated as a pale oil in 5% yield by chiral separation (CHIRALPAK AD-H column, Mobile phase: Hexane/EtOH/HOAc=80/20/0.1 (v/v/v)) from racemic I-409. The absolute configuration at the stereocenter was not established. MS (ESI, pos. ion) m/z: 275.2 (M+1). ¹H NMR (400 MHz, CDCl₃): δ 7.4 (s, 1H), 3.5 (m, 6H), 3.4 (s, 2H), 3.2 (s, 2H), 2.58 (m, 1H), 2.28 (m, 1H), 1.7 (m, 1H), 1.1 (m, 3H).

### The Synthesis of I-412 Involved 3 Steps as Depicted in the Following Scheme 133

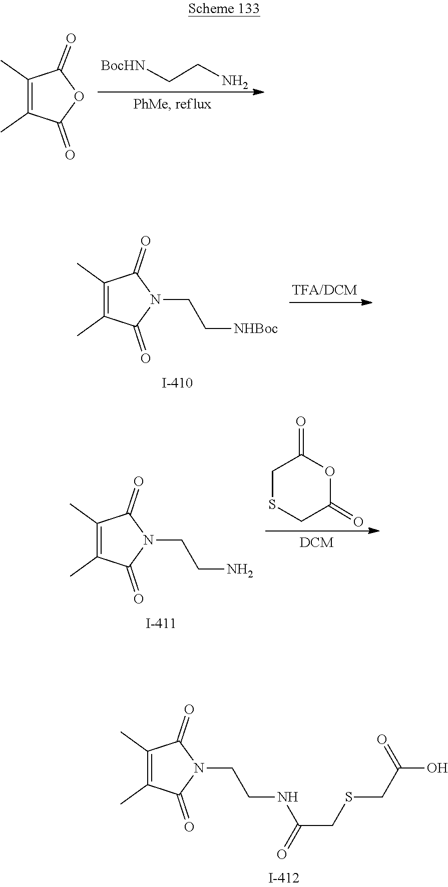

### 2-((2-((2-(3,4-dimethyl-2,5-dioxo-2,5-dihydro-1H-pyrrol-1-yl)ethyl)amino)-2-oxoethyl)thio) acetic acid (I-412)

### Step 1. tert-butyl (2-(3,4-dimethyl-2,5-dioxo-2,5-dihydro-1H-pyrrol-1-yl)ethyl)carbamate (I-410)

A solution of 3,4-dimethylfuran-2,5-dione (cas: 766-39-2, 600 mg, 4.76 mmol) and tert-butyl (2-aminoethyl)carbamate (cas: 57260-73-8, 1.1 g, 1.5 equiv.) in toluene (30 mL) was heated to reflux for 2 hours. The reaction mixture was then concentrated and purified by silica gel chromatography (Hexane:EtOAc=5:1) to afford tert-butyl (2-(3,4-dimethyl-2,5-dioxo-2,5-dihydro-1H-pyrrol-1-yl)ethyl)carbamate, I-410 as a yellow solid (700 mg, 55%). MS (ESI, pos. ion) m/z: 291.1 (M+23).

### Step 2: 1-(2-aminoethyl)-3,4-dimethyl-1H-pyrrole-2,5-dione (I-411)

To a solution tert-butyl (2-(3,4-dimethyl-2,5-dioxo-2,5-dihydro-1H-pyrrol-1-yl)ethyl)carbamate (700 mg, 2.61 mmol) in DCM (10 mL) was added TFA (2 mL). The reaction mixture was stirred at ambient temperature for 1 hour, then concentrated to afford the crude 1-(2-aminoethyl)-3,4-dimethyl-1H-pyrrole-2,5-dione TFA salt, I-411 as a brown oil (500 mg, 108%). MS (ESI, pos. ion) m/z: 169.1 (M+1).

### Step 3: 2-((2-((2-(3,4-dimethyl-2,5-dioxo-2,5-dihydro-1H-pyrrol-1-yl)ethyl)amino)-2-oxoethyl) thio) acetic acid (I-412)

A solution of 1-(2-aminoethyl)-3,4-dimethyl-1H-pyrrole-2,5-dione (0.4 g, 2.38 mmol) and thiodiglycolic anhydride (1.2 g, 4 equiv.) in DCM (5 mL) was stirred at ambient temperature overnight. The reaction was monitored by LC-MS. Upon completion of the reaction, the reaction mixture was concentrated and purified by preparative TLC to afford acid I-412 (100 mg, 14%) as a white solid. MS (ESI, pos. ion) m/z: 301.1 (M+1). ¹H NMR (400 MHz, CDCl₃): δ 8.28 (s, 1H), 3.70 (m, 2H), 3.50 (m, 2H), 3.39 (s, 2H), 3.36 (s, 1H), 3.32 (s, 2H), 1.96 (s, 6H).

### (S*)-I-413 and (R*)-I-413

The synthetic route for (S*)-I-413 and (R*)-I-413 was similar to the route for (R*)- and (S*)-I-234. The synthesis started with 5-methylpyrrolidin-2-one. The final target compounds were purified by preparative HPLC to afford 200 mg (55%) of racemic I-413 as a pale oil. Preparative chiral separation (CHIRALPAK AD column (Hexane/EtOH/AcOH=70/30/0.1)) of the racemate afforded the two enantiomers.

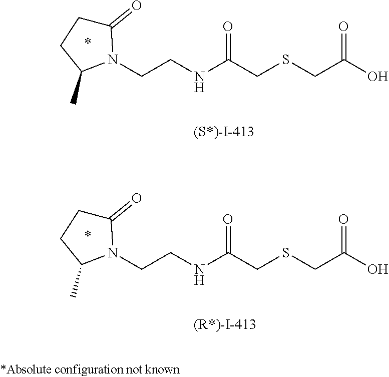 [SMILES: C[C@H]1CCC(=O)N1CCNC(=O)CSCC(=O)O ; C[C@@H]1CCC(=O)N1CCNC(=O)CSCC(=O)O]

### (S*)-I-413

After chiral HPLC purification, 30 mg of (S*)-I-413 (absolute configuration not determined) was obtained as a yellow oil. MS (ESI, pos. ion) m/z: 275.1 (M+1). ¹H NMR (400 MHz, CDCl₃): δ 7.50 (s, 1H), 6.81 (s, 1H), 3.96-3.84 (m, 1H), 3.77-3.67 (m, 1H), 3.71-3.58 (m, 1H), 3.36 (s, 2H), 3.29-3.19 (m, 2H), 3.28-3.23 (m, 1H), 3.18-3.08 (m, 1H), 2.51-2.37 (m, 2H), 2.26-2.16 (m, 1H), 1.72-1.53 (m, 1H), 1.26-1.22 (m, 3H).

### (R*)-I-413

After chiral HPLC purification, 41 mg of (R*)-I-413 (absolute configuration not determined) was obtained as a yellow oil. MS (ESI, pos. ion) m/z: 275.1 (M+1). ¹H NMR (400 MHz, CDCl₃): δ 7.39 (s, 1H), 3.96-3.88 (m, 1H), 3.88-3.79 (m, 1H), 3.73 (s, 1H), 3.36 (s, 2H), 3.29-3.19 (m, 3H), 3.15-3.07 (m, 1H), 2.47-2.37 (m, 2H), 2.27-2.19 (m, 1H), 1.67-1.56 (m, 1H), 1.26-1.24 (m, 3H).

### The Synthesis of Compound I-417 Involved 4 Steps as Depicted in the Following Scheme 134

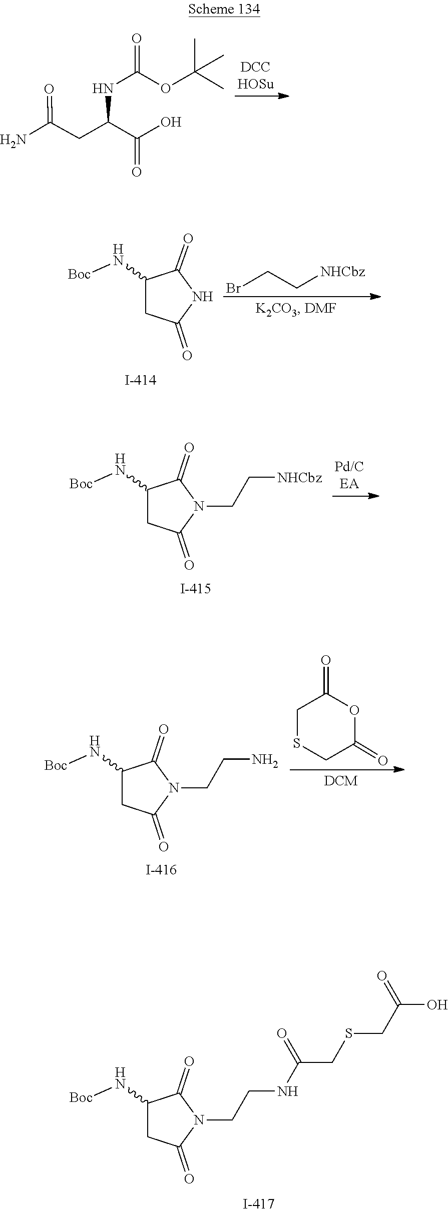

The chemical structure of the target compound was confirmed by 1HNMR and LC-MS with the purity >95% (HPLC 214 & 254 nm).

### (R)-2-((2-((2-(3-((tert-butoxycarbonyl)amino)-2,5-dioxopyrrolidin-1-yl)ethyl)amino)-2-oxoethyl)thio) acetic acid (I-417)

### Step 1: tert-butyl (2,5-dioxopyrrolidin-3-yl)carbamate (I-414)

To a stirred solution of (tert-butoxycarbonyl)-D-asparagine (cas: 75647-01-7, 1.0 g, 4.3 mmol) in DMF (7 mL) was added DCC (0.8 g, 1.0 equiv.) and HOSu (0.5 g, 1.0 equiv.). The reaction mixture was heated to 80° C. for 6 hours and then concentrated under reduced pressure. The residue was dissolved in EtOAc (10 mL) and filtered. The filtrate was washed with water, brine, dried with Na₂SO₄, and then concentrated under reduced pressure. The resulting residue was purified by column chromatography (EtOAc: Petroleum ether=1:1) to afford imide I-414 as a pale yellow solid (0.5 g, 53% yield). MS (ESI, pos. ion) m/z: 237.1 (M+23).

### Step 2: tert-butyl (1-(2-(((benzyloxy)carbonyl)amino)ethyl)-2,5-dioxopyrrolidin-3-yl)carbamate (I-415)

To a solution of tert-butyl (2,5-dioxopyrrolidin-3-yl) carbamate, I-414 (0.5 g, 2.3 mmol) and benzyl(2-bromoethyl)carbamate (cas: 53844-02-3, 890 mg, 1.5 equiv.) in DMF (5 mL) was added potassium carbonate (800 mg, 2.5 equiv.). The reaction mixture was heated to 90° C. for 12 hours. The reaction was then quenched with water (5 mL), and extracted with EtOAc (3×20 mL). The combined organic extracts were then washed with water (20 mL), brine (20 mL), dried with Na₂SO₄ and concentrated under reduced pressure. The residue was purified by column chromatography (EtOAc: Petroleum ether=1:1) to provide carbamate I-415 as a pale yellow oil (0.9 g, 100%). MS (ESI, pos. ion) m/z: 414.1 (M+23).

### Step 3: tert-butyl-(1-(2-aminoethyl)-2,5-dioxopyrrolidin-3-yl) carbamate (I-416)

To a solution of tert-butyl (1-(2-(((benzyloxy)carbonyl)amino)ethyl)-2,5-dioxopyrrolidin-3-yl) carbamate, I-415 (0.9 g, 2.3 mmol) in EtOAc (15 ml), was added 100 mg of Pd/C under H₂ (14 Psi). The reaction mixture was stirred at ambient temperature overnight then filtered, and the filtrate was concentrated to afford the crude tert-butyl-(1-(2-aminoethyl)-2,5-dioxopyrrolidin-3-yl) carbamate I-416 as brown oil (0.5 g, 84%). MS (ESI, pos. ion) m/z: 280.1 (M+23).

### Step 4: 2-((2-((2-(3-((tert-butoxycarbonyl)amino)-2,5-dioxopyrrolidin-1-yl)ethyl)amino)-2-oxoethyl)thio) acetic acid (I-417)

A solution of tert-butyl-(1-(2-aminoethyl)-2,5-dioxopyrrolidin-3-yl) carbamate, I-416 (0.1 g, 0.4 mmol) and thiodiglycolic anhydride (211 mg, 4 equiv.) in DCM (5 mL) was stirred at ambient temperature for 4 hours. The reaction was monitored by LC-MS. Upon completion of the reaction, the reaction mixture was concentrated, and the resulting residue was purified by preparative HPLC to afford I-417 (racemic) (39 mg, 25% yield). MS (ESI, pos. ion) m/z: 412.2 (M+23). ¹H NMR (400 MHz, CDCl₃): δ 7.34 (s, 1H), 5.62 (m, 1H), 4.36 (s, 1H), 3.72 (m, 1H), 3.57 (m, 1H), 3.45 (m, 3H), 3.31 (1H), 3.05 (m, 1H), 2.80 (s, 2H), 2.01 (s, 2H), 1.44 (s, 9H).

### The Synthesis of Compound I-421 Involved 4 Steps as Depicted in the Following Scheme 135

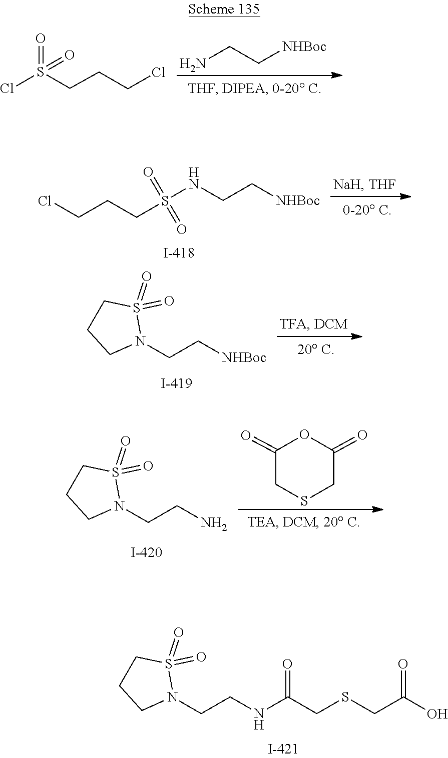

The chemical structure of the target compound was confirmed by 1HNMR and LC-MS with the purity >95% (HPLC 214 & 254 nm).

### Step 1: Tert-butyl (2-((3-chloropropyl)sulfonamido)ethyl)carbamate (I-418)

A solution of 3-chloropropane-1-sulfonyl chloride (cas: 1633-82-5, 1.22 g, 6.9 mmol) in anhydrous THF (5 mL) was added to a solution of tert-butyl (2-aminoethyl)carbamate (1 g, 6.25 mmol) and DIPEA (1.30 mL) in anhydrous THF (62 mL) at 0-5° C. The reaction mixture was was allowed to warm to ambient temperature and stirred for 8 hours. When LC-MS analysis indicated that the reaction was complete, H₂O (20 mL) was added, and the reaction mixture was extracted with EtOAc (20 mL×3). The combined organic extracts were concentrated under reduced pressure to give the crude product. The crude product was purified by silica gel chromatography (Petroleum ether:EtOAc=1:2) to provide I-418 (2.0 g, 96%) as a pale oil. MS (ESI, pos. ion) m/z: 323 (M+Na).

### Step 2: Tert-butyl (2-(1,1-dioxidoisothiazolidin-2-yl)ethyl)carbamate (I-419)

To a solution of tert-butyl (2-aminoethyl)carbamate, I-418 (2 g, 6.67 mmol) in anhydrous THF (70 mL) at 0-5° C. was added NaH (60% dispersion in mineral oil, 0.4 g 1.5 equiv.) in portions. The reaction was allowed to warm to ambient temperature and stirred for 8 hours. When LC-MS analysis indicated that the reaction was complete, H₂O (20 mL) was added and the reaction mixture was extracted with EtOAc (20 mL×3). The combined organic extracts were concentrated in vacuo to provide the crude product. The crude product was purified by silica gel chromatography (Petroleum Ether:EtOAc=2:1) to provide I-419 (0.15 g, 9% yield) as a pale oil. MS (ESI, pos. ion) m/z: 287 (M+Na).

### Step 3:2-(2-aminoethyl) isothiazolidine 1,1-dioxide (I-420)

A solution of tert-butyl (2-(1,1-dioxidoisothiazolidin-2-yl)ethyl)carbamate, I-419 (150 mg, 0.57 mmol) in TFA/DCM (1:3 v/v, 4 mL) was stirred at 25° C. for 2 hours. When LC-MS analysis indicated that the reaction was complete, the reaction mixture was concentrated in vacuo to give the crude amine TFA salt, I-420 as a pale oil (337 mg), which was used directly in the next step. MS (ESI, pos. ion) m/z: 165 (M+1).

### Step 4. 2-((2-((2-(1,1-dioxidoisothiazolidin-2-yl)ethyl)amino)-2-oxoethyl)thio) acetic acid (I-421)

To a solution of 2-(2-aminoethyl) isothiazolidine-1,1-dioxide (337 mg, 1.22 mmol) and thiodiglycolic anhydride (193 mg, 1.2 equiv.) in 5 mL of DCM was added TEA (24 mg, 0.244 mmol). The reaction mixture was stirred for 2 hours and then concentrated under reduced pressure to give the crude product. The crude product was purified by preparative HPLC (Mobile Phase: MeCN/H₂O (0.1% TFA), Gradient: 1-5% MeCN) to afford I-421 as a pale oil (50 mg, 14% yield). MS (ESI, pos. ion) m/z: 297.1 (M+1). ¹H NMR (400 MHz, DMSO) & 12.60 (s, 1H), 8.11 (m, 1H), 3.36 (s, 2H), 3.27-3.19 (m, 6H), 3.19-3.12 (m, 2H), 2.95 (m, 2H), 2.25-2.15 (m, 2H).

### I-425

### The Synthesis of Compound I-425 Involved 4 Steps as Depicted in the Following Scheme 136

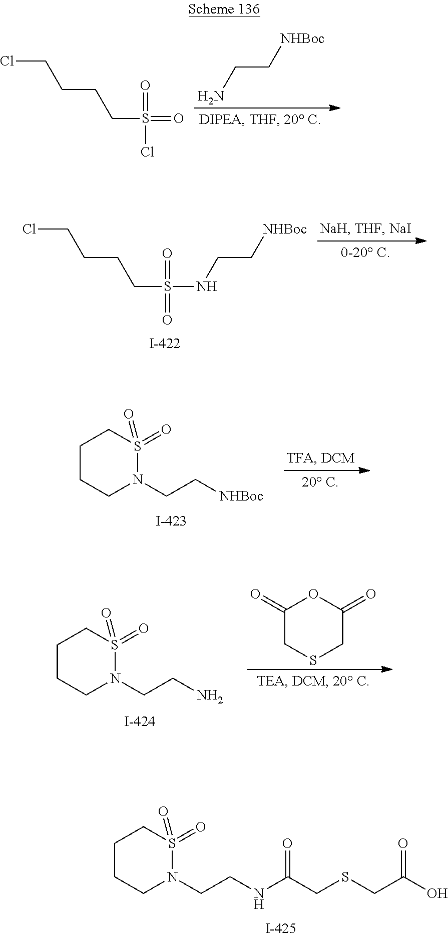

### 2-((2-((2-(1,1-dioxido-1,2-thiazinan-2-yl)ethyl)amino)-2-oxoethyl)thio) acetic acid (I-425)

The synthetic route for I-425 was similar to that used for the synthesis of I-421 and commenced with 4-chlorobutane-1-sulfonyl chloride (cas: 1633-84-7). 2-((2-((2-(1,1-dioxido-1,2-thiazinan-2-yl)ethyl)amino)-2-oxoethyl)thio)acetic acid, I-425 (pale oil, 50 mg, 15% yield) was isolated by preparative HPLC (Mobile Phase: MeCN/H₂O (with 0.1% TFA), Gradient: 10-20% MeCN). MS (ESI, pos. ion) m/z: 311.1 (M+1). 1HNMR (400 MHz, DMSO) § 12.57 (s, 1H), 8.13 (m, 1H), 3.37 (s, 2H), 3.34-3.30 (m, 2H), 3.21 (s, 2H), 3.18 (m, 2H), 3.11 (m, 2H), 3.08-3.03 (m, 2H), 2.06-1.95 (m, 2H), 1.54 (m, 3.7 Hz, 2H).

### Synthesis of I-426 was Depicted in Scheme 137

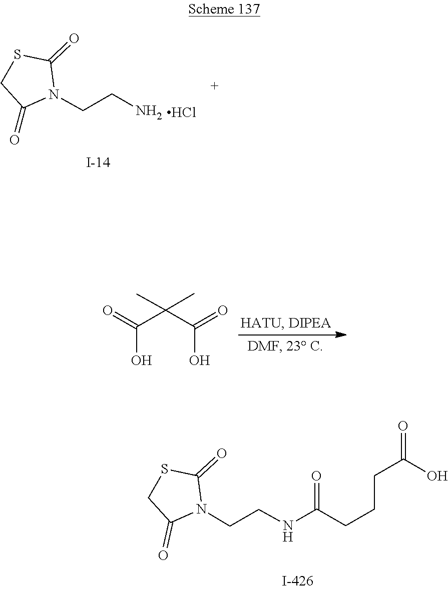

To a solution of amine salt I-14 (44.5 mg, 0.226 mmol), 2,2-dimethylmalonic acid (59.8 mg, 2.0 equiv.) and DIPEA (118 μL, 3.0 equiv.) in DMF (1 mL) was added HATU (129.1 mg, 1.5 equiv.). The reaction mixture was maintained at ambient temperature for 2 hours and was then purified by preparative HPLC (Mobile Phase: MeCN/H₂O with 0.1% TFA) to afford I-426 (8.6 mg, 14% yield).

### D. Examples of Compounds Described in this Invention

### General Procedure 4 (GP4): Solid Phase Synthesis of Peptides Utilizing Carboxylic Acid Building Blocks

### Example 1: Compound 83

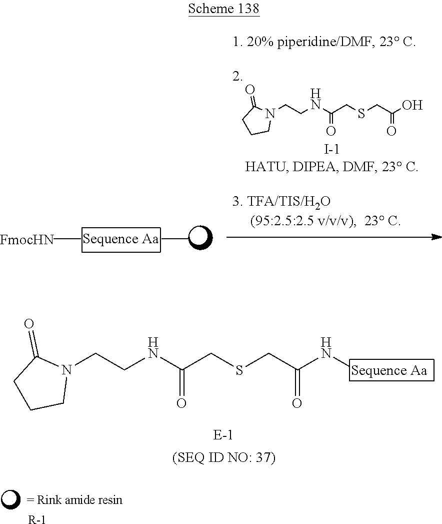

E-1 was synthesized as depicted in Scheme 138.

To Rink-amide resin R-1(0.125 mmol/g, 140.5 mg, 17.6 μmol) in a 6 mL polypropylene tube with an end-cap was added 20% v/v piperidine/DMF (2 mL). The tube was capped and agitated at ambient temperature for 30 minutes, then drained. The resin was washed with DMF (5×3 mL). A solution of carboxylic acid building block I-1(36.6 mg, 8.0 equiv.) in DMF (2.8 mL) was added to the resin, followed by DIPEA (61 μL, 20 equiv.). HATU (80 mg, 12.0 equiv.) was then added and the reaction mixture was agitated at ambient temperature for 18 hours. The reaction mixture was drained and the resin washed with DMF (5×3 mL), DCM (5×3 mL) and dried in vacuo for 30 minutes.

The resin was transferred to a 15 mL Falcon tube and 3 mL of cleavage reagent (95:2.5:2.5 v/v/v TFA/TIS/H₂O) was added. The reaction mixture was agitated at ambient temperature for 1 hour. The resin was filtered and washed with TFA (2×3 mL). The combined filtrate and washes were concentrated under reduced pressure to afford a residue, which was triturated with Et₂O (3 mL) to precipitate the peptide. The peptide was re-dissolved in glacial AcOH (2 mL) and purified by preparative HPLC (Phenomenex Jupiter 10 μM Proteo 90 Å LC column, 250×21.2 mm, with flow rate-15 mL/min, gradient of 0-100% acetonitrile in 25 mM aqueous ammonium acetate over 30 minutes) to afford 25.8 mg of E-1 as a white solid. ESI-MS (positive ionization) found 1149.7, [C₂₁₃H₃₂₅N₄₇O₆₄S]⁴⁺ calculated 1149.3.

### General Procedure 5 (GP5): Solid Phase Synthesis of Peptides Utilizing Pentafluorophenyl or N-Hydroxysuccinimide Ester Building Blocks

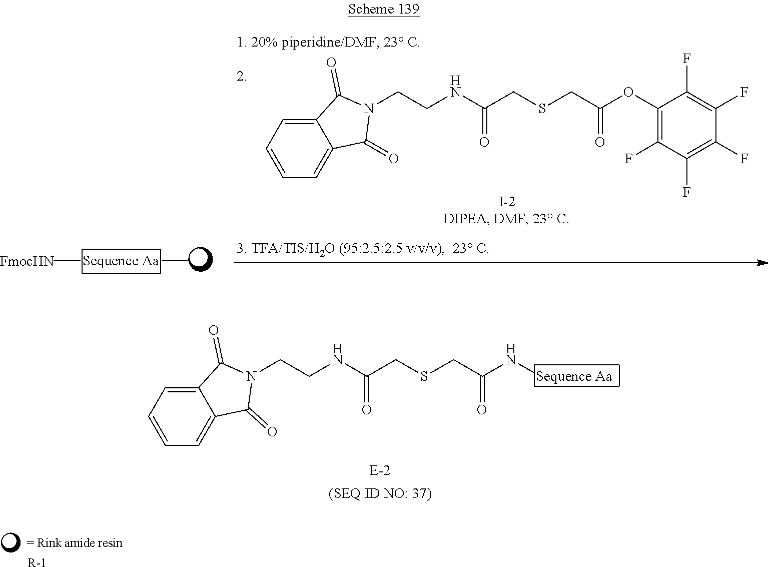

### Example 2: Compound 173

E-2 was synthesized as depicted in Scheme 139.

To Rink-amide resin (0.125 mmol/g, 107.8 mg, 13.5 μmol) in a 6 mL polypropylene tube with an end-cap was added 20% v/v piperidine/DMF (2 mL). The tube was capped, agitated at ambient temperature for 30 minutes, and then drained. The resin was washed with DMF (5×3 mL). A solution of pentafluorophenyl ester I-2(32.9 mg 5.0 equiv.) in DMF (1 mL) was added to the resin, followed by DIPEA (24 μL, 10 equiv.) and the reaction mixture was agitated at ambient temperature for 24 hours. The reaction mixture was drained. The resin was washed with DMF (5×3 mL), DCM (5×3 mL), and dried in vacuo for 30 minutes.

The resin was transferred to a 15 mL falcon tube and 3 mL of cleavage reagent (95:2.5:2.5 v/v/v TFA/TIS/H₂O) was added. The reaction mixture was agitated at ambient temperature for 3 hours. The resin was filtered and washed with TFA (2×0.5 mL). The combined filtrate and washes were concentrated under reduced pressure to afford a residue, which was triturated with Et₂O (2 mL) to precipitate the peptide. The peptide was re-dissolved in glacial AcOH (1.2 mL) and purified by preparative HPLC (Phenomenex Jupiter 10 μM Proteo 90 Å LC column, 250×21.2 mm, with flow rate-15 mL/min, gradient of 0-100% acetonitrile in 25 mM aqueous ammonium acetate over 30 minutes) to afford 9.4 mg of E-2 as a white solid. ESI-MS (positive ionization) found 1165.3, [C₂₁₇H₃₂₃N₄₇O₆₅S]⁴⁺ calculated 1164.8.

### Example 3: Compound 189

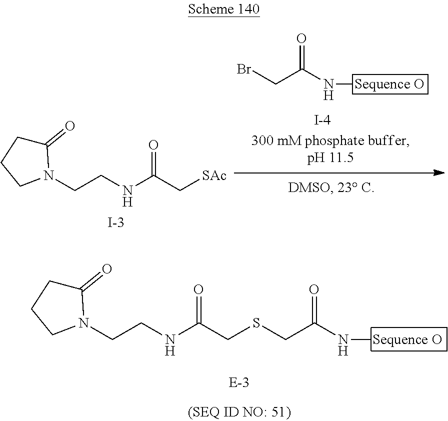

E-3 was synthesized as depicted in Scheme 140.

To peptide I-4 (0.25 mL of a 5 mM stock solution in DMSO, 1.2 μmol) was added I-3 (50 μL of a 50 mM stock solution in DMSO, 2.0 equiv.), followed by 300 mM sodium phosphate buffer, pH 11.5 (50 μL). The reaction mixture was agitated at ambient temperature for 16 hours and then purified by preparative HPLC (H₂O/MeCN with 0.1% TFA) to afford E-3 (4.3 mg) as a white solid. ESI-MS found 1374.3, C₁₈₆H₂₆₉N₄₆O₅₉S (M-3H) requires 1374.3.

### Example 4: Compound 190

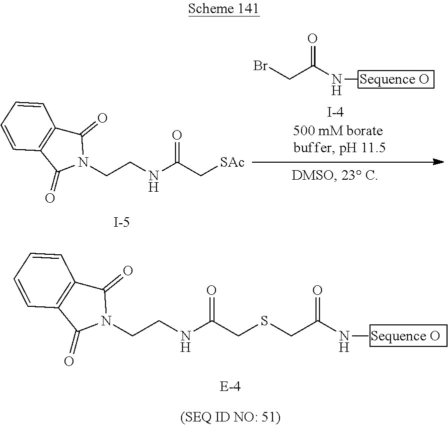

E-4 was synthesized as depicted in Scheme 141.

To peptide I-4 (0.25 mL of a 5 mM stock solution in DMSO, 1.2 μmol) was added I-5 (50 μL of a 50 mM stock solution in DMSO, 2.0 equiv.), followed by 500 mM borate buffer, pH 9.5 (25 μL). The reaction mixture was agitated at ambient temperature for 16 hours and then purified by preparative HPLC (H₂O/MeCN with 0.1% TFA) to afford E-4 (1.8 mg) as a white solid. ESI-MS found 1395.0, C₁₉₀H₂₆₇N₄₆O₆₀S (M−3H) requires 1395.0.

### Example 5: Compound 139

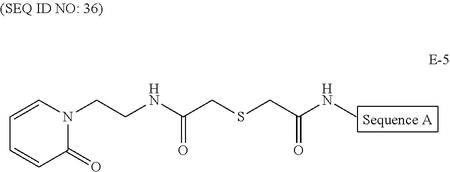 [SMILES: CNC(=O)CSCC(=O)NCCn1ccccc1=O]

E-5 was prepared according to GP4 using 24 mg of resin (estimated loading 0.18 mmol/g) and I-6 to afford 2.3 mg of E-5 as a white solid. ESI-MS found 1058.5, C₁₉₃H₂₇₈N₄₆O₆₀S (M−4H⁺) requires 1058.0.

### Example 6: Compound 118

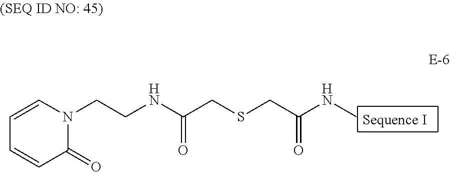 [SMILES: CNC(=O)CSCC(=O)NCCn1ccccc1=O]

E-6 was prepared according to GP4 using 30.9 mg of resin (estimated loading 0.15 mmol/g) and I-6 to afford 0.9 mg of E-6 as a white solid. ESI-MS found 1063.5, C₁₉₄H₂₈₈N₄₆O₆₀S (M+4H⁺) requires 1063.5.

### Example 7: Compound 52

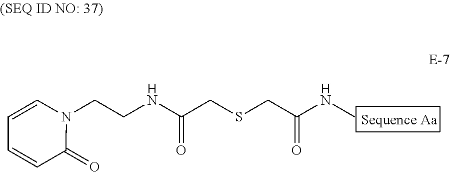 [SMILES: CNC(=O)CSCC(=O)NCCn1ccccc1=O]

E-7 was prepared according to GP4 using 43.3 mg of resin (estimated loading 0.125 mmol/g) and I-6 to afford 2.5 mg of E-7 as a white solid. ESI-MS found 1152.3, C₂₁₄H₃₂₃N₄₇O₆₄S (M+4H⁺) requires 1151.8.

### Example 8: Compound 121

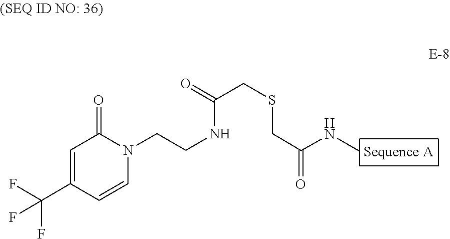 [SMILES: CNC(=O)CSCC(=O)NCCn1ccc(C(F)(F)F)cc1=O]

E-8 was prepared according to GP4 using 30.8 mg of resin (estimated loading 0.18 mmol/g) and I-7 to afford 2.4 mg of E-8 as a white solid. ESI-MS found 1077.6, C₁₉₄H₂₈₅FN₄₆O₆₀S (M+4H⁺) requires 1077.0.

### Example 9: Compound 117

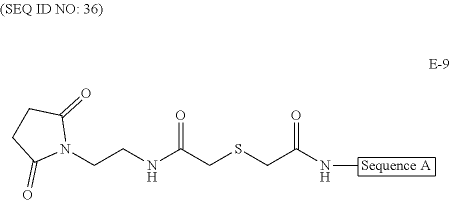 [SMILES: CNC(=O)CSCC(=O)NCCN1C(=O)CCC1=O]

E-9 was prepared according to GP4 using 30.3 mg of resin (estimated loading 0.18 mmol/g) and I-8 to afford 3.1 mg of E-9 as a white solid. ESI-MS found 1061.0, C₁₉₂H₂₈₆N₄₆O₆₁S (M+4H⁺) requires 1061.0.

### Example 10: Compound 114

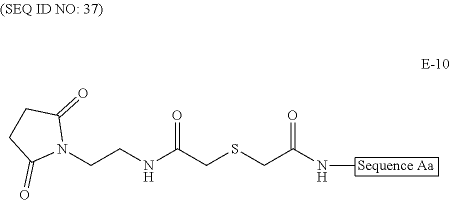 [SMILES: CNC(=O)CSCC(=O)NCCN1C(=O)CCC1=O]

E-10 was prepared according to GP4 using 40.1 mg of resin (estimated loading 0.125 mmol/g) and I-8 to afford 1.5 mg of E-10 as a white solid. ESI-MS found 1152.8, C₂₁₃H₃₂₃N₄₇O₆₅S (M+4H⁺) requires 1152.8.

### Example 11: Compound 53

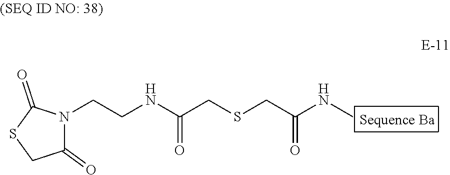 [SMILES: CNC(=O)CSCC(=O)NCCN1C(=O)CSC1=O]

E-11 was prepared according to GP4 using 67 mg of resin (estimated loading 0.08 mmol/g) and I-9 to afford 2.5 mg of E-11 as a white solid. ESI-MS found 1157.9, C₂₁₂H₃₂₁N₄₇O₆₅S₂ (M+4H⁺) requires 1157.3.

### Example 12: Compound 56

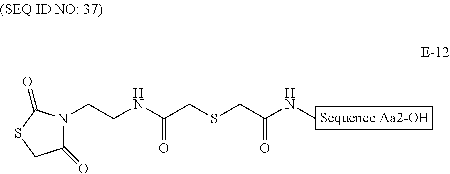 [SMILES: CNC(=O)CSCC(=O)NCCN1C(=O)CSC1=O]

E-12 was prepared according to GP4 using 55.6 mg of resin (estimated loading 0.15 mmol/g) and I-9 to afford 1.7 mg of E-12 as a white solid. ESI-MS found 1125.8, C₂₀₇H₃₁₃N₄₅O₆₃S₂ (M+4H⁺) requires 1125.3.

### Example 13: Compound 60

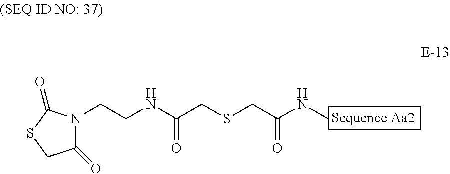 [SMILES: CNC(=O)CSCC(=O)NCCN1C(=O)CSC1=O]

E-13 was prepared according to GP4 using 53.3 mg of resin (estimated loading 0.18 mmol/g) and I-9 to afford 5.0 mg of E-13 as a white solid. ESI-MS found 1125.4, C₂₀₇H₃₁₄N₄₆O₆₂S₂ (M+4H⁺) requires 1125.1.

### Example 14: Compound 76

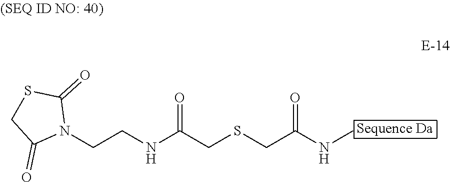 [SMILES: CNC(=O)CSCC(=O)NCCN1C(=O)CSC1=O]

E-14 was prepared according to GP4 using 41.3 mg of resin (estimated loading 0.11 mmol/g) and I-9 to afford 0.5 mg of E-14 as a white solid. ESI-MS found 1238.2, C₂₂₂H₃₄₈N₅₆O₆₆S3 (M+4H⁺) requires 1237.6.

### Example 15: Compound 73

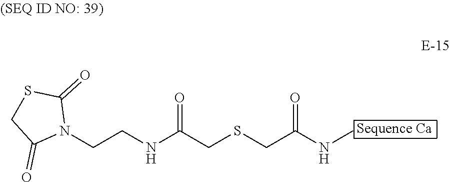 [SMILES: CNC(=O)CSCC(=O)NCCN1C(=O)CSC1=O]

E-15 was prepared according to GP4 using 40.4 mg of resin (estimated loading 0.06 mmol/g) and I-9 to afford 0.8 mg of E-15 as a white solid. ESI-MS found 1135.3, C₂₀₆H₃₁₉N₄₇O₆₄S₂ (M+4H⁺) requires 1134.8.

### Example 16: Compound 70

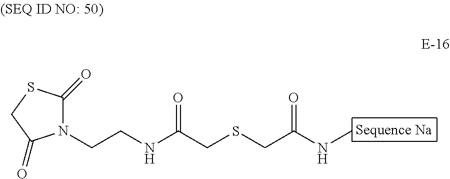 [SMILES: CNC(=O)CSCC(=O)NCCN1C(=O)CSC1=O]

E-16 was prepared according to GP4 using 56.5 mg of resin (estimated loading 0.18 mmol/g) and I-9 to afford 1.1 mg of E-16 as a white solid. ESI-MS found 1161.2, C₂₁₃H₃₂₃N₄₇O₆₅S₂ (M+4H⁺) requires 1160.8.

### Example 17: Compound 71

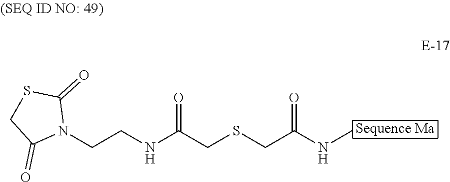 [SMILES: CNC(=O)CSCC(=O)NCCN1C(=O)CSC1=O]

E-17 was prepared according to GP4 using 68 mg of resin (estimated loading 0.16 mmol/g) and I-9 to afford 3.3 mg of E-17 as a white solid. ESI-MS found 1139.3, C₂₀₅H₃₁₅N₄₇O₆₄S₃ (M+4H⁺) requires 1138.8.

### Example 18: Compound 72

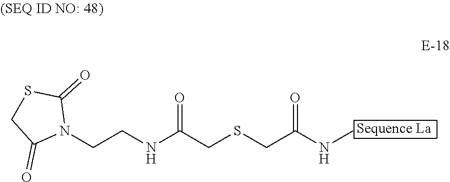 [SMILES: CNC(=O)CSCC(=O)NCCN1C(=O)CSC1=O]

E-18 was prepared according to GP4 using 56.9 mg of resin (estimated loading 0.17 mmol/g) and I-9 to afford 0.7 mg of E-18 as a white solid. ESI-MS found 1153.4, C₂₀₇H₃₁₈N₄₈O₆₅S₃ (M+4H⁺) requires 1153.1.

### Example 19: Compound 137

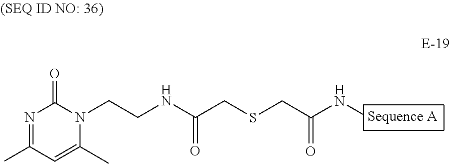

E-19 was prepared according to GP4 using 27.3 mg of resin (estimated loading 0.18 mmol/g) and I-10 to afford 0.7 mg of E-19 as a white solid. ESI-MS found 1065.8, C₁₉₄H₂₈₁N₄₇O₆₀S (M−4H)⁻ requires 1065.3.

### Example 20: Compound 138

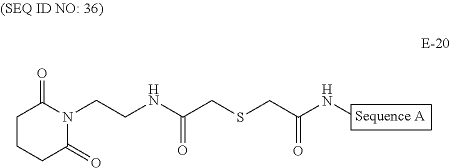 [SMILES: CNC(=O)CSCC(=O)NCCN1C(=O)CCCC1=O]

E-20 was prepared according to GP4 using 29 mg of resin (estimated loading 0.18 mmol/g) and I-11 to afford 3.0 mg of E-20 as a white solid. ESI-MS found 1063.0, C₁₉₃H₂₈₀N₄₆O₆₁S (M−4H)⁻ requires 1062.5.

### Example 21: Compound 123

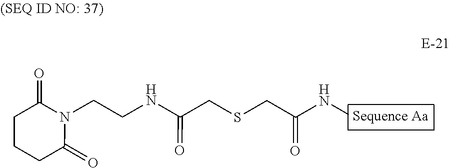 [SMILES: CNC(=O)CSCC(=O)NCCN1C(=O)CCCC1=O]

E-21 was prepared according to GP4 using 108.5 mg of resin (estimated loading 0.125 mmol/g) and I-11 to afford 9.7 mg of E-21 as a white solid. ESI-MS found 1156.8, C₂₁₄H₃₂₅N₄₇O₆₅S (M+4H⁺) requires 1156.3.

### Example 22: Compound 120

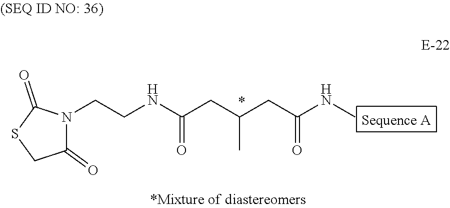 [SMILES: CNC(=O)CC(C)CC(=O)NCCN1C(=O)CSC1=O]

E-22 was prepared according to GP4 using 31.5 mg of resin (estimated loading 0.18 mmol/g) and I-12 to afford 1.8 mg of E-22 as a white solid. ESI-MS found 1065.1, C₁₉₃H₂₈₈N₄₆O₆₁S (M+4H⁺) requires 1064.5.

### Example 23: Compound 122

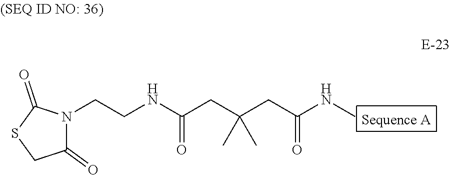 [SMILES: CNC(=O)CC(C)(C)CC(=O)NCCN1C(=O)CSC1=O]

E-23 was prepared according to GP4 using 29.4 mg of resin (estimated loading 0.18 mmol/g) and I-13 to afford 4.2 mg of E-23 as a white solid. ESI-MS found 1068.6, C₁₉₄H₂₉₀N₄₆O₆₁S (M+4H⁺) requires 1068.0.

### Example 24: Compound 170

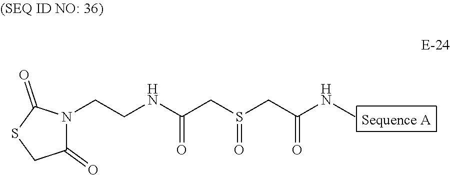 [SMILES: CNC(=O)CS(=O)CC(=O)NCCN1C(=O)CSC1=O]

E-24 was prepared according to GP4 using 26.9 mg of resin (estimated loading 0.15 mmol/g) and I-15 to afford 3.3 mg of E-24 as a white solid. ESI-MS found 1068.0, C₁₉₁H₂₇₆N₄₆O₆₂S₂ (M−4H)⁻ requires 1067.5.

### Example 25: Compound 131

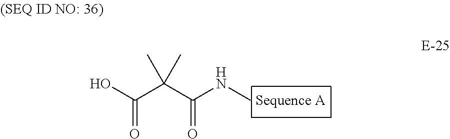

E-25 was prepared according to GP3 using 29.1 mg of resin (estimated loading 0.18 mmol/g) and diester I-16 to afford 2.1 mg of E-25 as a white solid. ESI-MS found 1024.0, C₁₈₇H₂₇₂N₄₄O₆₀ (M−4H)⁻ requires 1023.5.

### Example 26: Compound 143

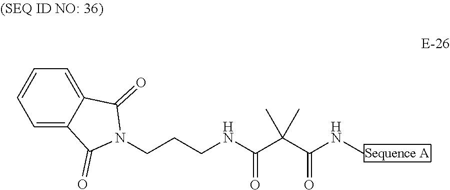 [SMILES: CNC(=O)C(C)(C)C(=O)NCCCN1C(=O)c2ccccc2C1=O]

E-26 was prepared according to GP5 using 28.7 mg of resin (estimated loading 0.18 mmol/g) and I-21 to afford 3.4 mg of E-26 as a white solid. ESI-MS found 1070.5, C₁₉₈H₂₈₂N₄₆O₆₁ (M−4H)⁻ requires 1070.0.

### Example 27: Compound 142

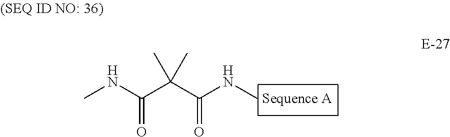

E-27 was prepared according to GP5 using 24.7 mg of resin (estimated loading 0.18 mmol/g) and I-22 to afford 2.5 mg of E-27 as a white solid. ESI-MS found 1027.3, C₁₈₈H₂₇₅N₄₅O₅₉ (M−4H)⁻ requires 1026.8.

### Example 28: Compound 141

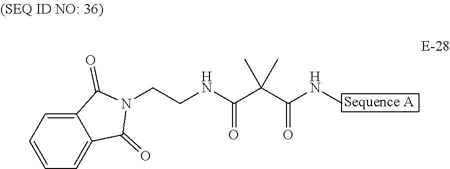 [SMILES: CNC(=O)C(C)(C)C(=O)NCCN1C(=O)c2ccccc2C1=O]

E-28 was prepared according to GP5 using 26.1 mg of resin (estimated loading 0.18 mmol/g) and I-23 to afford 1.8 mg of E-28 as a white solid. ESI-MS found 1074.0, C₁₉₉H₂₈₄N₄₆O₆₁ (M−4H)⁻ requires 1073.5.

### Example 29: Compound 140

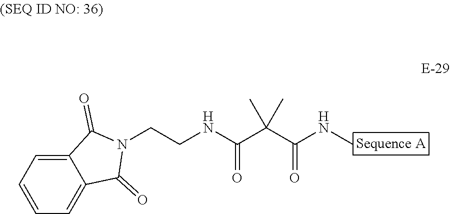 [SMILES: CNC(=O)C(C)(C)C(=O)NCCN1C(=O)c2ccccc2C1=O]

E-29 was prepared according to GP5 using 23.5 mg of resin (estimated loading 0.18 mmol/g) and I-24 to afford 0.5 mg of E-29 as a white solid. ESI-MS found 1067.0, C₁₉₇H₂₈₀N₄₆O₆₁ (M−4H⁺) requires 1066.5.

### Example 30: Compound 116

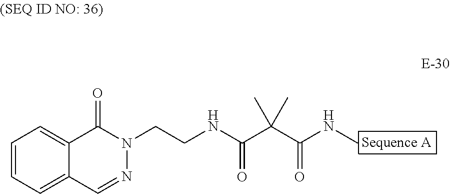 [SMILES: CNC(=O)C(C)(C)C(=O)NCCn1ncc2ccccc2c1=O]

E-30 was prepared according to GP5 using 24.9 mg of resin (estimated loading 0.18 mmol/g) and I-25 to afford 1.4 mg of E-30 as a white solid. ESI-MS found 1068.7, C₁₉₇H₂₈₉N₄₇O₆₀ (M+4H⁺) requires 1068.3.

### Example 31: Compound 127

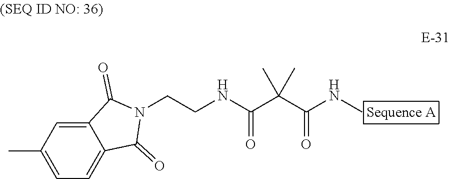

E-31 was prepared according to GP5 using 28.4 mg of resin (estimated loading 0.18 mmol/g) and I-26 to afford 2.4 mg of E-31 as a white solid. ESI-MS found 1070.5, C₁₉₈H₂₈₂N₄₆O₆₁ (M−4H)⁻ requires 1070.0.

### Example 32: Compound 129

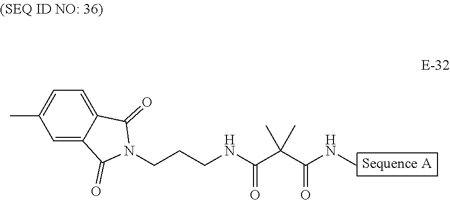

E-32 was prepared according to GP5 using 26.4 mg of resin (estimated loading 0.18 mmol/g) and I-27 to afford 5.5 mg of E-32 as a white solid. ESI-MS found 1074.0, C₁₉₉H₂₈₄N₄₆O₆₁ (M−4H)⁻ requires 1073.5.

### Example 33: Compound 132

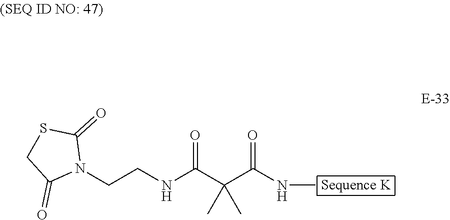 [SMILES: CNC(=O)C(C)(C)C(=O)NCCN1C(=O)CSC1=O]

E-33 was prepared according to GP5 using 27.1 mg of resin (estimated loading 0.2 mmol/g) and I-28 to afford 3.1 mg of E-33 as a white solid. ESI-MS found 1070.0, C₁₉₅H₂₈₄N₄₆O₆₁S (M−4H)⁻ requires 1069.5.

### Example 34: Compound 134

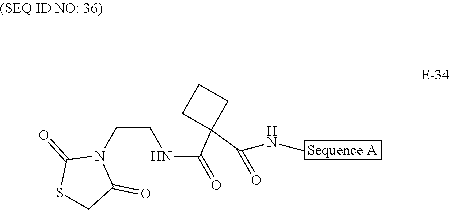 [SMILES: CNC(=O)C1(C(=O)NCCN2C(=O)CSC2=O)CCC1]

E-34 was prepared according to GP5 using 28 mg of resin (estimated loading 0.18 mmol/g) and I-29 to afford 3.0 mg of E-34 as a white solid. ESI-MS found 1062.5, C₁₉₃H₂₇₈N₄₆O₆₁S (M−4H)⁻ requires 1149.8.

### Example 35: Compound 135

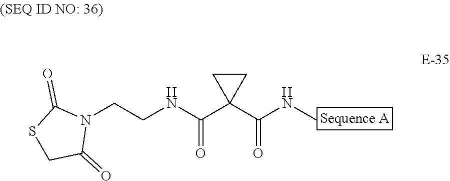 [SMILES: CNC(=O)C1(C(=O)NCCN2C(=O)CSC2=O)CC1]

E-35 was prepared according to GP5 using 25.8 mg of resin (estimated loading 0.18 mmol/g) and I-30 to afford 2.4 mg of E-35 as a white solid. ESI-MS found 1059.0, C₁₉₂H₂₇₆N₄₆O₆₁S (M−4H)⁻ requires 1058.5.

### Example 36: Compound 19

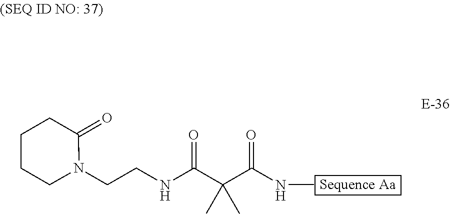 [SMILES: CNC(=O)C(C)(C)C(=O)NCCN1CCCCC1=O]

E-36 was prepared according to GP5 using 39.7 mg of resin (estimated loading 0.125 mmol/g) and I-31 to afford 5.9 mg of E-36 as a white solid. ESI-MS found 1148.9, C₂₁₅H₃₂₉N₄₇O₆₄ (M+4H⁺) requires 1148.3.

### Example 37: Compound 119

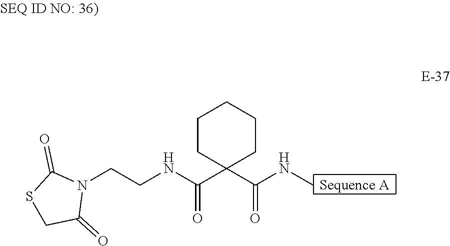 [SMILES: CNC(=O)C1(C(=O)NCCN2C(=O)CSC2=O)CCCCC1]

E-37 was prepared according to GP5 using 34 mg of resin (estimated loading 0.18 mmol/g) and I-32 to afford 5.2 mg of E-37 as a white solid. ESI-MS found 1071.6, C₁₉₅H₂₉₀N₄₆O₆₁S (M+4H⁺) requires 1071.0.

### Example 38: Compound 124

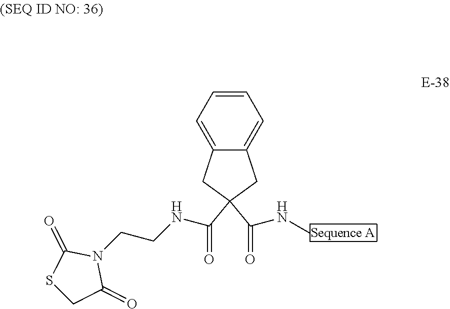 [SMILES: CNC(=O)C1(C(=O)NCCN2C(=O)CSC2=O)Cc2ccccc2C1]

E-38 was prepared according to GP5 using 21.6 mg of resin (estimated loading 0.18 mmol/g) and I-33 to afford 3.5 mg of E-38 as a white solid. ESI-MS found 1080.0, C₁₉₈H₂₈₈N₄₆O₆₁S (M+4H⁺) requires 1079.5.

### Example 39: Compound 80

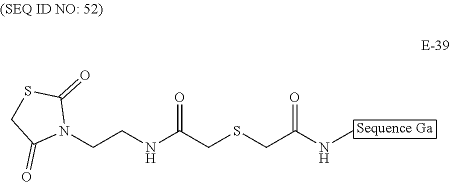 [SMILES: CNC(=O)CSCC(=O)NCCN1C(=O)CSC1=O]

E-39 was prepared according to GP5 using 51.5 mg of resin (estimated loading 0.14 mmol/g) and I-9 to afford 0.8 mg of E-39 as a white solid. ESI-MS found 1164.9, C₂₁₄H₃₂₅N₄₇O₆₅S₂ (M+4H⁺) requires 1164.3.

### Example 40: Compound 196

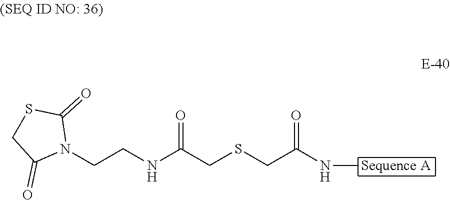 [SMILES: CNC(=O)CSCC(=O)NCCN1C(=O)CSC1=O]

E-40 was prepared according to GP5 using 26.2 mg of resin (estimated loading 0.38 mmol/g) and I-34 to afford 3.3 mg of E-40 as a white solid. ESI-MS found 1064.0, C₁₉₁H₂₇₆N₄₆O₆₁S₂ (M−4H)⁻ requires 1063.5.

### Example 41: Compound 195

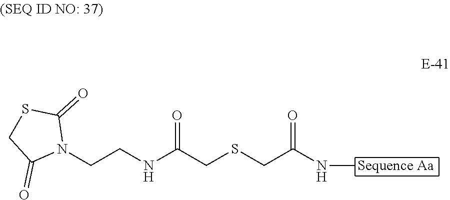 [SMILES: CNC(=O)CSCC(=O)NCCN1C(=O)CSC1=O]

E-41 was prepared according to GP5 using 109.9 mg of resin (estimated loading 0.125 mmol/g) and I-34 to afford 7.3 mg of E-41 as a white solid. ESI-MS found 1156.1, C₂₁₂H₃₁₃N₄₇O₆₅S₂ (M−4H)⁻ requires 1155.3.

### Example 42: Compound 147

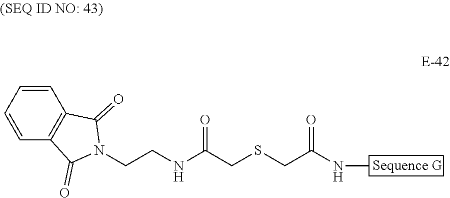 [SMILES: CNC(=O)CSCC(=O)NCCN1C(=O)c2ccccc2C1=O]

Peptide E-42 was prepared from 39 mg of resin (approximate loading 0.18 mmol/g) using I-35 and GP5 to afford E-42 as a white solid. ESI-MS found 1078.5, C₁₉₈H₂₈₂N₄₆O₆₁S (M−4H)⁻ requires 1078.0.

### Example 43: Compound 145

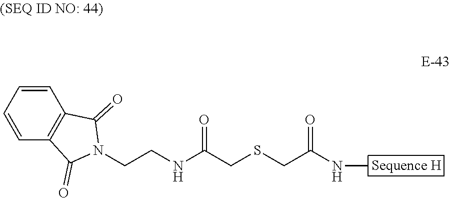 [SMILES: CNC(=O)CSCC(=O)NCCN1C(=O)c2ccccc2C1=O]

Peptide E-43 was prepared from 36 mg of resin (approximate loading 0.18 mmol/g) using I-35 and GP5 to afford E-43 as a white solid. ESI-MS found 1067.5, C₁₉₇H₂₈₂N₄₆O₅₉S (M−4H)⁻ requires 1067.0.

### Example 44: Compound 144

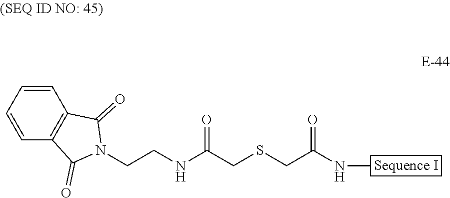 [SMILES: CNC(=O)CSCC(=O)NCCN1C(=O)c2ccccc2C1=O]

Peptide E-44 was prepared from 36 mg of resin (approximate loading 0.18 mmol/g) using I-35 and GP5 to afford E-44 as a white solid. ESI-MS found 1075.0, C₁₉₇H₂₈₀N₄₆O₆₁S (M−4H)⁻ requires 1074.5.

### Example 45: Compound 133

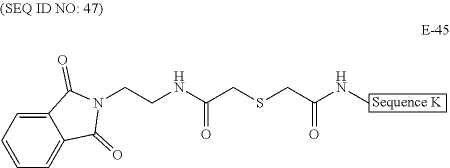 [SMILES: CNC(=O)CSCC(=O)NCCN1C(=O)c2ccccc2C1=O]

Peptide E-45 was prepared from 27.9 mg of resin (approximate loading 0.2 mmol/g) using I-35 and GP5 to afford 2.3 mg of E-45 as a white solid. ESI-MS found 1082.0, C₁₉₉H₂₈₄N₄₆O₆₁S (M−4H)⁻ requires 1081.5.

### Example 46: Compound 149

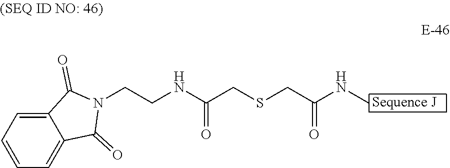 [SMILES: CNC(=O)CSCC(=O)NCCN1C(=O)c2ccccc2C1=O]

Peptide E-46 was prepared from 32 mg of resin (approximate loading 0.17 mmol/g) using I-35 and GP5 to afford 4.7 mg of E-46 as a white solid. ESI-MS found 1075.3, C₁₉₇H₂₇₉N₄₅O₆₂S (M−4H)⁻ requires 1074.8.

### Example 47: Compound 150

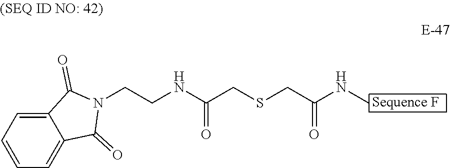 [SMILES: CNC(=O)CSCC(=O)NCCN1C(=O)c2ccccc2C1=O]

Peptide E-47 was prepared from 32.6 mg of resin (approximate loading 0.17 mmol/g) using I-35 and GP5 to afford 4.8 mg of E-47 as a white solid. ESI-MS found 1071.7, C₁₉₅H₂₇₃N₄₅O₆₃S (M−4H)⁻ requires 1071.2.

### Example 48: Compound 151

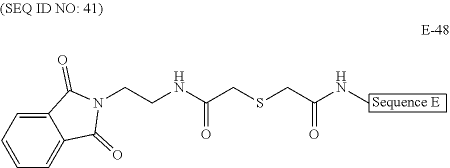 [SMILES: CNC(=O)CSCC(=O)NCCN1C(=O)c2ccccc2C1=O]

Peptide E-48 was prepared from 30.2 mg of resin (approximate loading 0.19 mmol/g) using I-35 and GP5 to afford 5.4 mg of E-48 as a white solid. ESI-MS found 1059.0, C₁₉₃H₂₈₀N₄₆O₆₀S (M−4H)⁻ requires 1058.5.

### Example 49: Compound 193

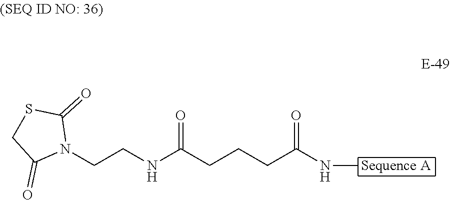 [SMILES: CNC(=O)CCCC(=O)NCCN1C(=O)CSC1=O]

E-49 was prepared according to GP5 using 50 mg of resin (estimated loading 0.18 mmol/g) and I-36 to afford 7.7 mg of E-49 as a white solid. ESI-MS found 1059.5, C₁₉₂H₂₇₈N₄₆O₆₁S (M−4H)⁻ requires 1059.0.

### Example 50: Compound 155

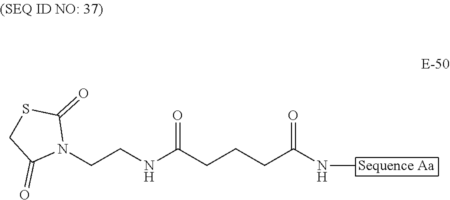 [SMILES: CNC(=O)CCCC(=O)NCCN1C(=O)CSC1=O]

E-50 was prepared according to GP5 using 52.9 mg of resin (estimated loading 0.15 mmol/g) and I-36 to afford E-50 as a white solid. ESI-MS found 1151.3, C₂₁₂H₃₂₃N₄₇O₆₄S (M−4H)⁻ requires 1150.8.

### Example 51: Compound 192

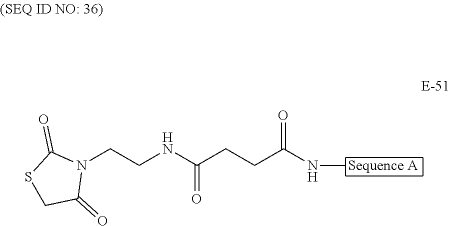 [SMILES: CNC(=O)CCC(=O)NCCN1C(=O)CSC1=O]

E-51 was prepared according to GP5 using 50 mg of resin (estimated loading 0.18 mmol/g) and I-37 to afford 5.9 mg of E-51 as a white solid. ESI-MS found 1056.0, C₁₉₁H₂₇₆N₄₆O₆₁S (M−4H)⁻ requires 1055.5.

### Example 52: Compound 194

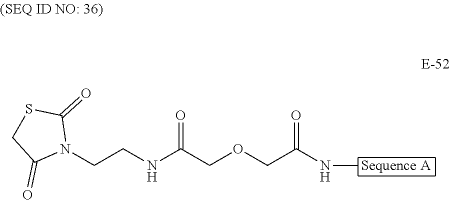 [SMILES: CNC(=O)COCC(=O)NCCN1C(=O)CSC1=O]

E-52 was prepared according to GP5 using 50 mg of resin (estimated loading 0.18 mmol/g) and I-38 to afford 3.1 mg of E-52 as a white solid. ESI-MS found 1060.0, C₁₉₁H₂₇₆N₄₆O₆₂S (M−4H)⁻ requires 1059.5.

### Example 53: Compound 191

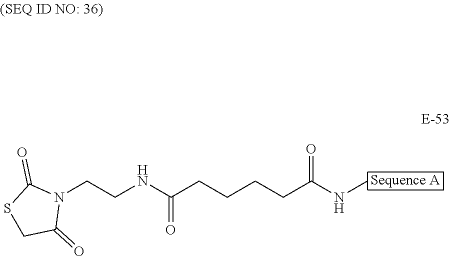 [SMILES: CNC(=O)CCCCC(=O)NCCN1C(=O)CSC1=O]

E-53 was prepared according to GP5 using 50 mg of resin (estimated loading 0.18 mmol/g) and I-39 to afford 7.8 mg of E-53 as a white solid. ESI-MS found 1063.0, C₁₉₃H₂₈₀N₄₆O₆₁S (M−4H)⁻ requires 1062.5.

### Example 54: Compound 187

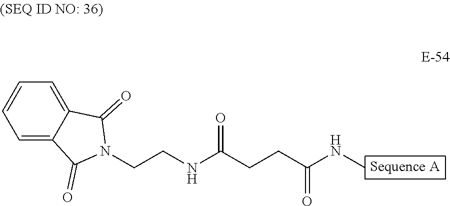 [SMILES: CNC(=O)CCC(=O)NCCN1C(=O)c2ccccc2C1=O]

E-54 was prepared according to GP5 using 22 mg of resin (estimated loading 0.5 mmol/g) and I-40 to afford 1.8 mg of E-54 as a white solid. ESI-MS found 1063.5, C₁₉₆H₂₇₈N₄₆O₆₁ (M−4H)⁻ requires 1063.0.

### Example 55: Compound 188

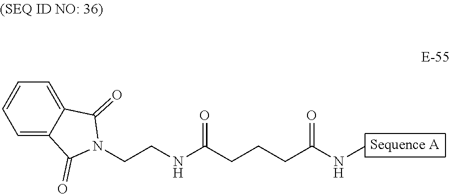 [SMILES: CNC(=O)CCCC(=O)NCCN1C(=O)c2ccccc2C1=O]

E-55 was prepared according to GP5 using 21.3 mg of resin (estimated loading 0.5 mmol/g) and I-41 to afford 1.0 mg of E-55 as a white solid. ESI-MS found 1067.0, C₁₉₇H₂₈₀N₄₆O₆₁ (M−4H)⁻ requires 1066.5.

### Example 56: Compound 184

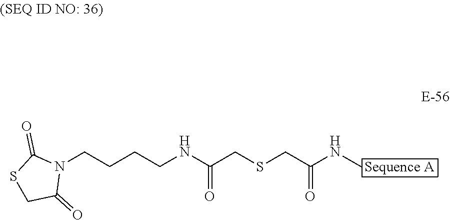 [SMILES: CNC(=O)CSCC(=O)NCCCCN1C(=O)CSC1=O]

E-56 was prepared according to GP5 using 13.6 mg of resin (estimated loading 0.5 mmol/g) and I-42 to afford 0.8 mg of E-56 as a white solid. ESI-MS found 1071.0, C₁₉₃H₂₈₀N₄₆O₆₁S₂ (M−4H)⁻ requires 1070.5.

### Example 57: Compound 186

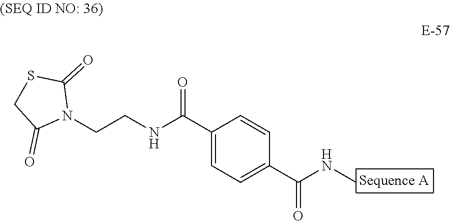 [SMILES: CNC(=O)c1ccc(C(=O)NCCN2C(=O)CSC2=O)cc1]

E-57 was prepared according to GP5 using 16.1 mg of resin (estimated loading 0.5 mmol/g) and I-44 to afford 0.4 mg of E-57 as a white solid. ESI-MS found 1068.0, C₁₉₅H₂₇₆N₄₆O₆₁S (M−4H)⁻ requires 1067.5.

### Example 58: Compound 181

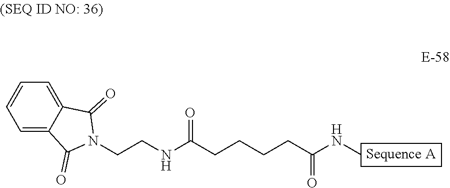 [SMILES: CNC(=O)CCCCC(=O)NCCN1C(=O)c2ccccc2C1=O]

E-58 was prepared according to GP5 using 20.7 mg of resin (estimated loading 0.5 mmol/g) and I-48 to afford 0.3 mg of E-58 as a white solid. ESI-MS found 1070.5, C₁₉₈H₂₈₂N₄₆O₆₁ (M−4H)⁻ requires 1070.0.

### Example 59: Compound 180

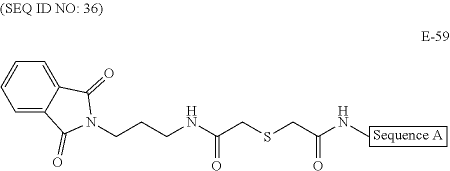 [SMILES: CNC(=O)CSCC(=O)NCCCN1C(=O)c2ccccc2C1=O]

E-59 was prepared according to GP5 using 13.6 mg of resin (estimated loading 0.5 mmol/g) and I-51 to afford 0.6 mg of E-59 as a white solid. ESI-MS found 1075.0, C₁₉₇H₂₈₀N₄₆O₆₁S (M−4H)⁻ requires 1074.5.

### Example 60: Compound 169

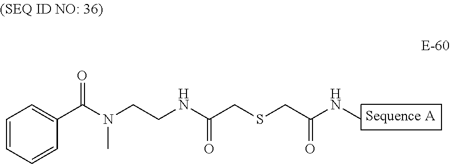 [SMILES: CNC(=O)CSCC(=O)NCCN(C)C(=O)c1ccccc1]

E-60 was prepared according to GP5 using 30.5 mg of resin (estimated loading 0.15 mmol/g) and I-53 to afford 3.6 mg of E-60 as a white solid. ESI-MS found 1068.5, C₁₉₆H₂₈₂N₄₆O₆₀S (M−4H)⁻ requires 1068.0.

### Example 61: Compound 81

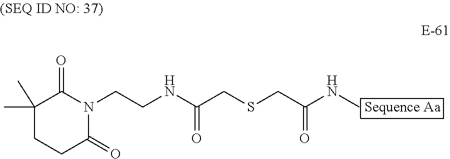

E-61 was prepared according to GP4 using 36.9 mg of resin (estimated loading 0.125 mmol/g) and I-55 to afford 5.1 mg of E-61 as a white solid. ESI-MS found 1163.8, C₂₁₆H₃₂₉N₄₇O₆₅S (M+4H⁺) requires 1163.3.

### Example 62: Compound 185

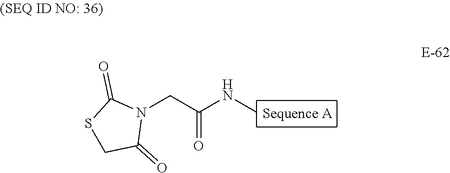

E-62 was prepared according to GP4 using 22 mg of resin (estimated loading 0.5 mmol/g) and commercially available 2-(2,4-dioxothiazolidin-3-yl)acetic acid to afford 0.8 mg of E-62 as a white solid. ESI-MS found 1034.7, C₁₈₇H₂₆₉N₄₅O₆₀S (M−4H)⁻ requires 1034.2.

### Example 63: Compound 178

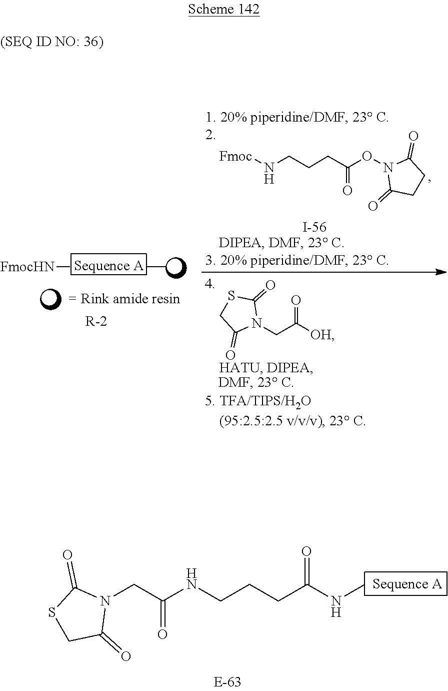

E-63 was synthesized as depicted in Scheme 142.

Resin R-2(29.2 mg, approximate loading 0.39 mmol/g, 11.3 μmol) was treated with 20% piperidine/DMF (3×5 min×1 mL) for Fmoc deprotection. The resin was drained and washed with DMF (5×1 mL). A solution of ester I-56 (23.9 mg, 5.0 equiv.) in DMF (0.6 mL) was added, followed by DIPEA (19.7 μL, 10.0 equiv.) and the reaction mixture was agitated at ambient temperature for 16 hours. The resin was then drained and washed with DMF (5×) and DCM (5×) and then dried in vacuo. The resin was treated with 20% piperidine/DMF (3×5 min×1 mL) for Fmoc deprotection. The resin was drained and washed with DMF (5×1 mL). A solution of 2-(2,4-dioxothiazolidin-3-yl)acetic acid (9.9 mg, 5.0 equiv.) in DMF (0.6 mL) was added, followed by DIPEA (23.6 μL, 12.0 equiv.) and HATU (34.4 mg, 8.0 equiv.) and the reaction mixture was agitated at ambient temperature for 16 hours. The resin was then drained and washed with DMF (5×) and DCM (5×) and then dried in vacuo. Resin cleavage was performed with TFA/TIPS/H₂O (95:2.5:2.5 v/v/v, 1 mL) at ambient temperature for 1 hour. The reaction mixture was filtered and the resin was washed with TFA (2×1 mL). The combined filtrate and washes were concentrated. The residue was triturated with Et₂O (2 mL) and the supernatant was discarded. The residue was dissolved in AcOH (1 mL) and purified by preparative HPLC (25 mM NH₄OAc/MeCN, 0-100% gradient over 30 minutes) to afford 1.6 mg of peptide E-63 as a white solid. ESI-MS found 1056.0, C₁₉₁H₂₇₆N₄₆O₆₁S (M−4H)⁻ requires 1055.5.

### Example 64: Compound 177

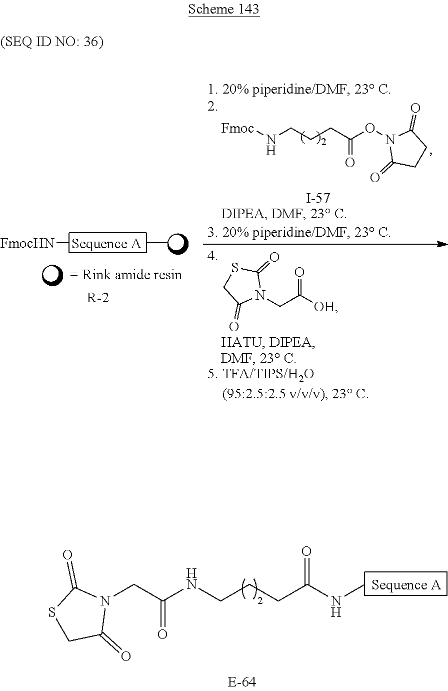

E-64 was synthesized as depicted in Scheme 143.

Resin R-2(36.1 mg, approximate loading 0.39 mmol/g, 14.1 μmol) was treated with 20% piperidine/DMF (3×5 min×1 mL) for Fmoc deprotection. The resin was drained and washed with DMF (5×1 mL). A solution of ester I-57 (30.7 mg, 5.0 equiv.) in DMF (0.6 mL) was added, followed by DIPEA (24.5 μL, 10.0 equiv.) and the reaction mixture was agitated at ambient temperature for 16 hours. The resin was then drained and washed with DMF (5×) and DCM (5×) and then dried in vacuo. The resin was treated with 20% piperidine/DMF (3×5 min×1 mL) for Fmoc deprotection. The resin was drained and washed with DMF (5×1 mL). A solution of 2-(2,4-dioxothiazolidin-3-yl)acetic acid (12.3 mg, 5.0 equiv.) in DMF (0.6 mL) was added, followed by DIPEA (29.4 μL, 12.0 equiv.) and HATU (42.8 mg, 8.0 equiv.) and the reaction mixture was agitated at ambient temperature for 16 hours. The resin was then drained and washed with DMF (5×) and DCM (5×) and then dried in vacuo. Resin cleavage was performed with TFA/TIPS/H₂O (95:2.5:2.5 v/v/v, 1 mL) at ambient temperature for 1 hour. The reaction mixture was filtered and the resin was washed with TFA (2×1 mL). The combined filtrate and washes were concentrated. The residue was triturated with Et₂O (2 mL) and the supernatant was discarded. The residue was dissolved in AcOH (1 mL) and purified by preparative HPLC (25 mM NH₄OAc/MeCN, 0-100% gradient over 30 minutes) to afford 1.2 mg of peptide E-64 as a white solid. ESI-MS found 1059.5, C₁₉₂H₂₇₈N₄₆O₆₁S (M−4H)⁻ requires 1059.0.

### Example 65: Compound 93

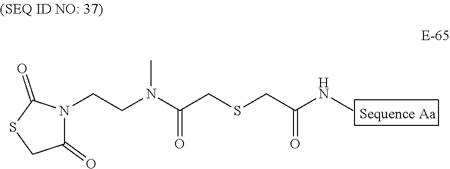 [SMILES: CNC(=O)CSCC(=O)N(C)CCN1C(=O)CSC1=O]

E-65 was prepared according to GP4 using 49.4 mg of resin (estimated loading 0.125 mmol/g) and I-60 to afford 5.8 mg of E-65 as a white solid. ESI-MS found 1161.4, C₂₁₃H₃₂₃N₄₇O₆₅S₂ (M+4H⁺) requires 1160.8.

### Example 66: Compound 148

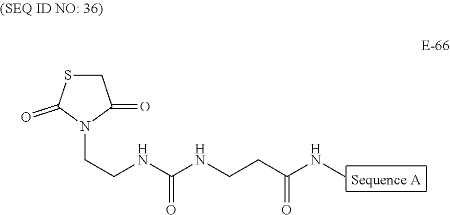 [SMILES: CNC(=O)CCNC(=O)NCCN1C(=O)CSC1=O]

E-66 was prepared according to GP4 using 28.6 mg of resin (estimated loading 0.18 mmol/g) and I-61 to afford 2.4 mg of E-66 as a white solid. ESI-MS found 1059.8, C₁₉₁H₂₇₇N₄₇O₆₁S (M−4H)⁻ requires 1059.3.

### Example 67: Compound 152

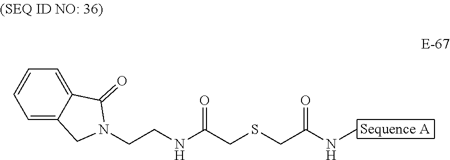 [SMILES: CNC(=O)CSCC(=O)NCCN1Cc2ccccc2C1=O]

E-67 was prepared according to GP4 using 26.5 mg of resin (estimated loading 0.18 mmol/g) and I-62 to afford E-67 as a white solid. ESI-MS found 1068.0, C₁₉₆H₂₈₀N₄₆O₆₀S (M−4H)⁻ requires 1067.5.

### Example 68: Compound 109

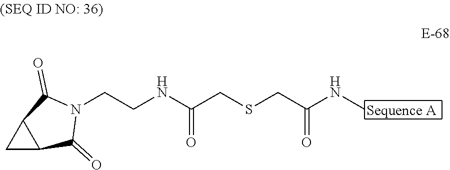 [SMILES: CNC(=O)CSCC(=O)NCCN1C(=O)[C@H]2C[C@H]2C1=O]

E-68 was prepared according to GP4 using 30.2 mg of resin (estimated loading 0.18 mmol/g) and I-64 to afford 4.6 mg of E-68 as a white solid. ESI-MS found 1064.0, C₁₉₃H₂₈₆N₄₆O₆₁S (M+4H⁺) requires 1064.0.

### Example 69: Compound 113

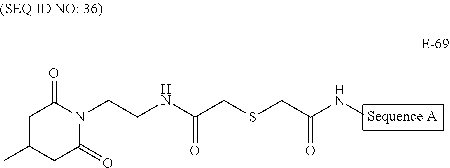

E-69 was prepared according to GP4 using 27.8 mg of resin (estimated loading 0.18 mmol/g) and I-66 to afford 2.9 mg of E-69 as a white solid. ESI-MS found 1067.9, C₁₉₄H₂₉₀N₄₆O₆₁S (M+4H⁺) requires 1068.0.

### Example 70: Compound 105

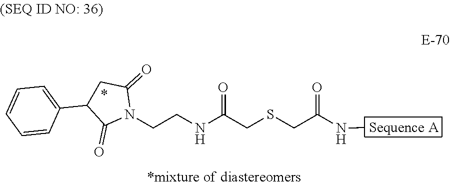 [SMILES: CNC(=O)CSCC(=O)NCCN1C(=O)CC(c2ccccc2)C1=O]

Peptide E-70 was prepared from 35 mg of resin (approximate loading 0.18 mmol/g) using I-67 and GP4 to afford 2.4 mg of E-70 as a white solid. ESI-MS found 1080.0, C₁₉₈H₂₉₀N₄₆O₆₁S (M+4H⁺) requires 1080.0.

### Example 71: Compound 99

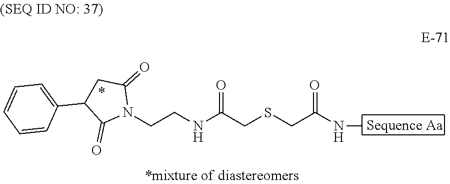 [SMILES: CNC(=O)CSCC(=O)NCCN1C(=O)CC(c2ccccc2)C1=O]

Peptide E-71 was prepared from 35 mg of resin (approximate loading 0.125 mmol/g) using I-67 and GP4 to afford 1.5 mg of E-71 as a white solid. ESI-MS found 1171.9, C₂₁₉H₃₂₇N₄₇O₆₅S (M+4H⁺) requires 1171.8.

### Example 72: Compound 136

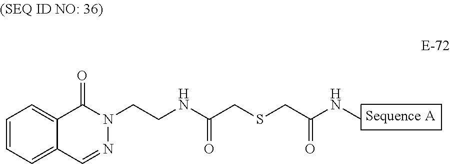 [SMILES: CNC(=O)CSCC(=O)NCCn1ncc2ccccc2c1=O]

E-72 was prepared according to GP4 using 24.4 mg of resin (estimated loading 0.18 mmol/g) and I-68 to afford 2.5 mg of E-72 as a white solid. ESI-MS found 1071.3, C₁₉₆H₂₇₉N₄₇O₆₀S (M−4H)⁻ requires 1070.8.

### Example 73: Compound 17

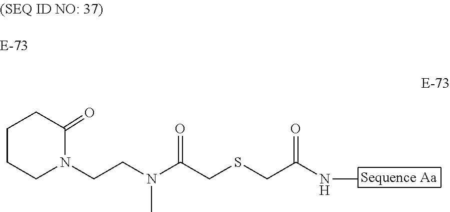 [SMILES: CNC(=O)CSCC(=O)N(C)CCN1CCCCC1=O]

E-73 was prepared according to GP4 using 46.2 mg of resin (estimated loading 0.125 mmol/g) and I-69 to afford 6.6 mg of E-73 as a white solid. ESI-MS found 1156.8, C₂₁₅H₃₂₉N₄₇O₆₄S (M+4H⁺) requires 1156.3.

### Example 74: Compound 13

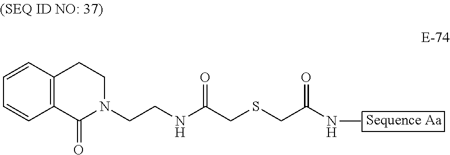 [SMILES: CNC(=O)CSCC(=O)NCCN1CCc2ccccc2C1=O]

Peptide E-74 was prepared from 30 mg of resin (approximate loading 0.125 mmol/g) using I-70 and GP4 to afford 2.2 mg of E-74 as a white solid. ESI-MS found 1165.6, C₂₁₈H₃₂₇N₄₇O₆₄S (M+4H⁺) requires 1164.8.

### Example 75: Compound 84

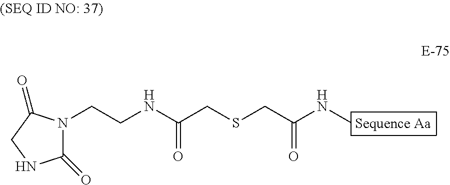 [SMILES: CNC(=O)CSCC(=O)NCCN1C(=O)CNC1=O]

E-75 was prepared according to GP4 using 35.8 mg of resin (estimated loading 0.125 mmol/g) and I-71 to afford 3.2 mg of E-75 as a white solid. ESI-MS found 1153.5, C₂₁₂H₃₂₃N₄₈O₆₅S (M+4H⁺) requires 1153.1.

### Example 76: Compound 87

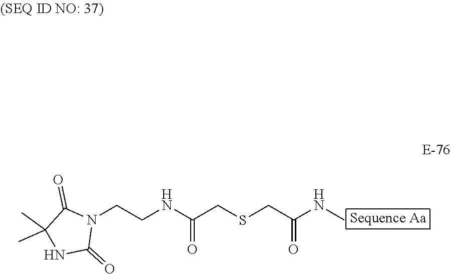

E-76 was prepared according to GP4 using 34.9 mg of resin (estimated loading 0.125 mmol/g) and I-72 to afford 3.5 mg of E-76 as a white solid. ESI-MS found 1160.5, C₂₁₄H₃₂₆N₄₈O₆₅S (M+4H⁺) requires 1160.1.

### Example 77: Compound 90

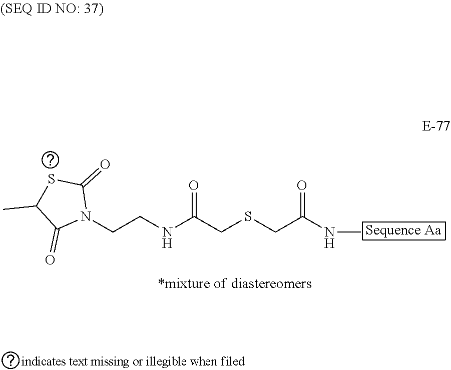

Peptide E-77 was prepared from 37 mg of resin (approximate loading 0.125 mmol/g) using I-74 and GP1 to afford 3.1 mg of E-77 as a white solid. ESI-MS found 1161.4, C₂₁₃H₃₂₃N₄₇O₆₅S₂ (M+4H⁺) requires 1160.8.

### Example 78: Compound 94

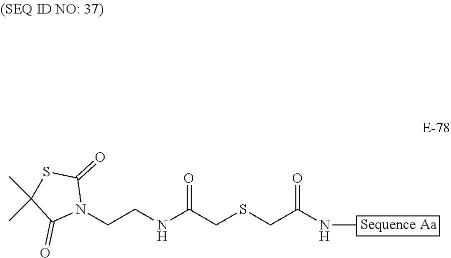

Peptide E-78 was prepared from 30 mg of resin (approximate loading 0.125 mmol/g) using I-76 and GP4 to afford 2.2 mg of E-78 as a white solid. ESI-MS found 1164.9, C₂₁₄H₃₂₅N₄₇O₆₅S₂ (M+4H⁺) requires 1164.3.

### Example 79: Compound 66

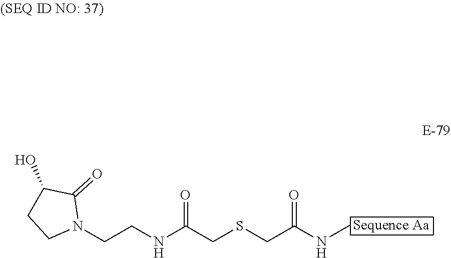 [SMILES: CNC(=O)CSCC(=O)NCCN1CC[C@H](O)C1=O]

E-79 was prepared according to GP4 using 38.4 mg of resin (estimated loading 0.125 mmol/g) and I-79 to afford 4.2 mg of E-79 as a white solid. ESI-MS found 1153.8, C₂₁₃H₃₂₅N₄₇O₆₅S (M+4H⁺) requires 1153.3.

### Example 80: Compound 47

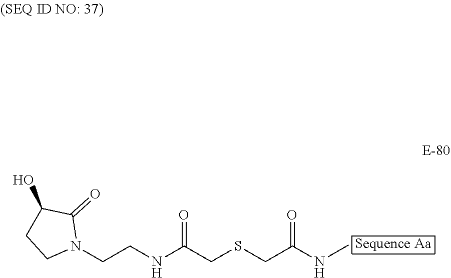 [SMILES: CNC(=O)CSCC(=O)NCCN1CC[C@@H](O)C1=O]

E-80 was prepared according to GP4 using 39.8 mg of resin (estimated loading 0.125 mmol/g) and I-80 to afford 2.5 mg of E-80 as a white solid. ESI-MS found 1153.8, C₂₁₃H₃₂₅N₄₇O₆₅S (M+4H⁺) requires 1153.3.

### Example 81: Compound 88

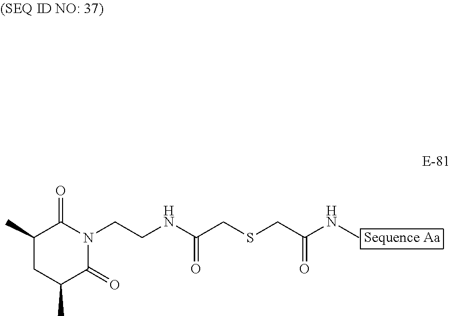

E-81 was prepared according to GP4 using 37.6 mg of resin (estimated loading 0.125 mmol/g) and I-82 to afford 2.1 mg of E-81 as a white solid. ESI-MS found 1163.8, C₂₁₆H₃₂₉N₄₇O₆₅S (M+4H⁺) requires 1163.3.

### Example 82: Compound 35

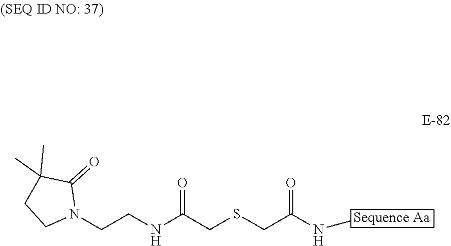

E-82 was prepared according to GP4 using 49.9 mg of resin (estimated loading 0.125 mmol/g) and I-85 to afford 6.5 mg of E-82 as a white solid. ESI-MS found 1156.9, C₂₁₅H₃₂₉N₄₇O₆₄S (M+4H⁺) requires 1156.3.

### Example 83: Compound 41

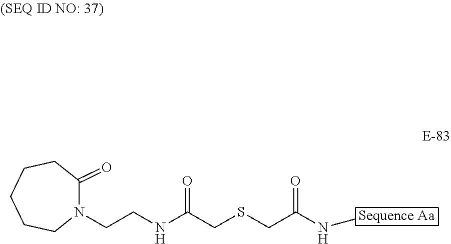 [SMILES: CNC(=O)CSCC(=O)NCCN1CCCCCC1=O]

E-83 was prepared according to GP4 using 50.8 mg of resin (estimated loading 0.125 mmol/g) and I-87 to afford 6.0 mg of E-83 as a white solid. ESI-MS found 1157.0, C₂₁₅H₃₂₉N₄₇O₆₄S (M+4H⁺) requires 1156.3.

### Example 84: Compound 92

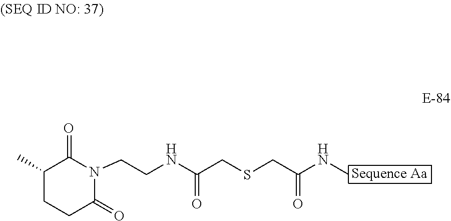

E-84 was prepared according to GP4 using 39.3 mg of resin (estimated loading 0.125 mmol/g) and I-89 to afford 2.9 mg of E-84 as a white solid. ESI-MS found 1160.4, C₂₁₅H₃₂₇N₄₇O₆₅S (M+4H⁺) requires 1159.8.

### Example 85: Compound 85

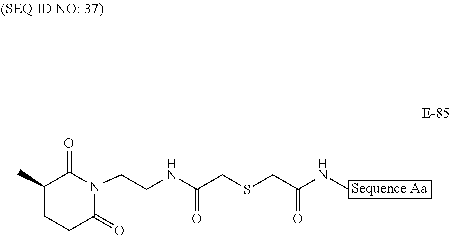

E-85 was prepared according to GP4 using 41.1 mg of resin (estimated loading 0.125 mmol/g) and I-90 to afford 3.7 mg of E-85 as a white solid. ESI-MS found 1160.4, C₂₁₅H₃₂₇N₄₇O₆₅S (M+4H⁺) requires 1159.8.

### Example 86: Compound 97

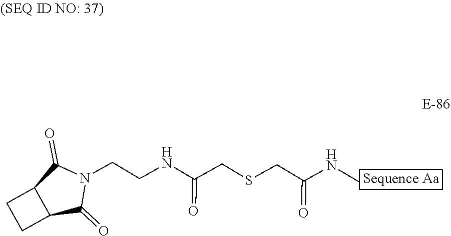 [SMILES: CNC(=O)CSCC(=O)NCCN1C(=O)[C@H]2CC[C@H]2C1=O]

E-86 was prepared according to GP4 using 30.9 mg of resin (estimated loading 0.125 mmol/g) and I-92 to afford 3.1 mg of E-86 as a white solid. ESI-MS found 1159.7, C₂₁₅H₃₂₅N₄₇O₆₅S (M+4H⁺) requires 1159.3.

### Example 87: Compound 125

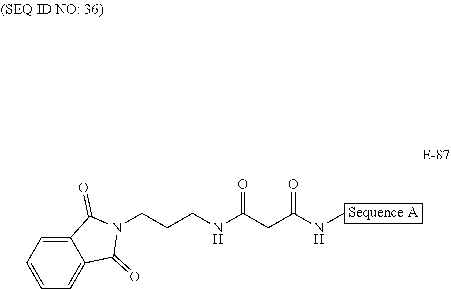 [SMILES: CNC(=O)CC(=O)NCCCN1C(=O)c2ccccc2C1=O]

E-87 was prepared according to GP4 using 21.9 mg of resin (estimated loading 0.18 mmol/g) and I-94 to afford 2.2 mg of E-87 as a white solid. ESI-MS found 1063.5, C₁₉₆H₂₇₈N₄₆O₆₁ (M−4H)⁻ requires 1063.0.

### Example 88: Compound 128

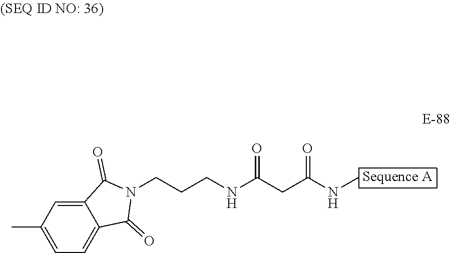

E-88 was prepared according to GP4 using 31 mg of resin (estimated loading 0.18 mmol/g) and I-96 to afford 3.4 mg of E-88 as a white solid. ESI-MS found 1067.0, C₁₉₇H₂₈₀N₄₆O₆₁ (M−4H)⁻ requires 1066.5.

### Example 89: Compound 126

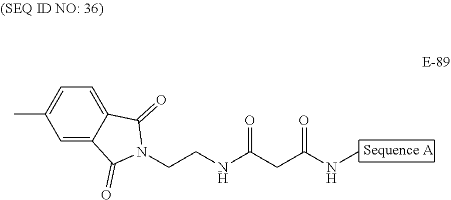

E-89 was prepared according to GP4 using 28.1 mg of resin (estimated loading 0.18 mmol/g) and I-98 to afford 2.5 mg of E-89 as a white solid. ESI-MS found 1063.5, C₁₉₆H₂₇₈N₄₆O₆₁ (M−4H)⁻ requires 1063.0.

### Example 90: Compound 91

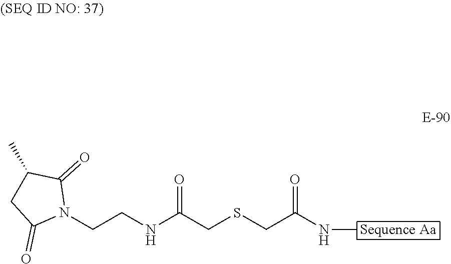 [SMILES: CNC(=O)CSCC(=O)NCCN1C(=O)C[C@H](C)C1=O]

E-90 was prepared according to GP4 using 35.5 mg of resin (estimated loading 0.125 mmol/g) and I-101 to afford 4.8 mg of E-90 as a white solid. ESI-MS found 1156.8, C₂₁₄H₃₂₅N₄₇O₆₅S (M+4H⁺) requires 1156.3.

### Example 91: Compound 89

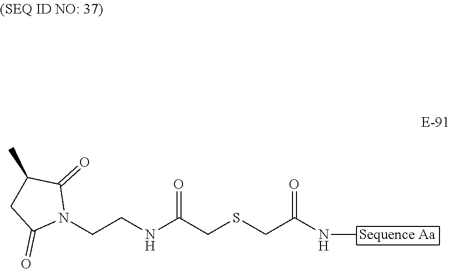 [SMILES: CNC(=O)CSCC(=O)NCCN1C(=O)C[C@@H](C)C1=O]

E-91 was prepared according to GP4 using 35.3 mg of resin (estimated loading 0.125 mmol/g) and I-100 to afford 3.2 mg of E-91 as a white solid. ESI-MS found 1156.8, C₂₁₄H₃₂₅N₄₇O₆₅S (M+4H⁺) requires 1156.3.

### Example 92: Compound 98

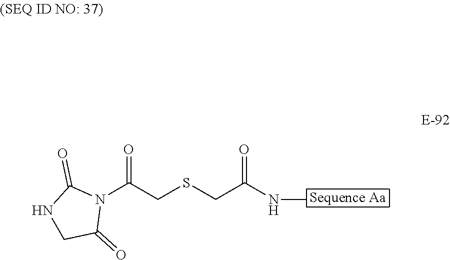 [SMILES: CNC(=O)CSCC(=O)N1C(=O)CNC1=O]

Peptide E-92 was prepared from 35 mg of resin (approximate loading 0.125 mmol/g) using I-104 and GP4 to afford 3.7 mg of E-92 as a white solid. ESI-MS found 1139.3, C₂₁₀H₃₁₉N₄₇O₆₄S (M+4H⁺) requires 1138.8.

### Example 93: Compound 103

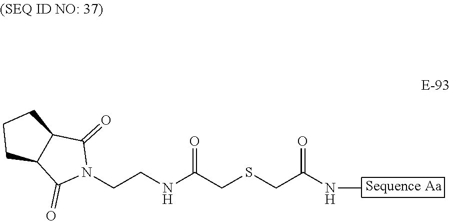 [SMILES: CNC(=O)CSCC(=O)NCCN1C(=O)[C@H]2CCC[C@H]2C1=O]

Peptide E-93 was prepared from 38 mg of resin (approximate loading 0.125 mmol/g) using I-106 and GP4 to afford 2.0 mg of E-93 as a white solid. ESI-MS found 1162.8, C₂₁₆H₃₂₇N₄₇O₆₅S (M+4H⁺) requires 1162.8.

### Example 94: Compound 106

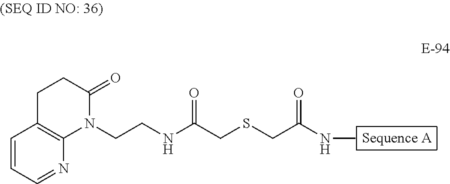 [SMILES: CNC(=O)CSCC(=O)NCCN1C(=O)CCc2cccnc21]

Peptide E-94 was prepared from 44 mg of resin (approximate loading 0.18 mmol/g) using I-109 and GP5 to afford 4.1 mg of E-94 as a white solid. ESI-MS found 1073.3, C₁₉₆H₂₈₉N₄₇O₆₀S (M+4H⁺) requires 1073.2.

### Example 95: Compound 107

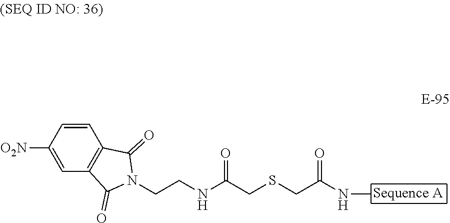 [SMILES: CNC(=O)CSCC(=O)NCCN1C(=O)c2ccc([N+](=O)[O-])cc2C1=O]

Peptide E-95 was prepared from 34 mg of resin (approximate loading 0.18 mmol/g) using I-111 and GP5 to afford 4.9 mg of E-95 as a white solid. ESI-MS found 1084.2, C₁₉₆H₂₈₅N₄₇O₆₃S (M+4H⁺) requires 1084.2.

### Example 96: Compound 108

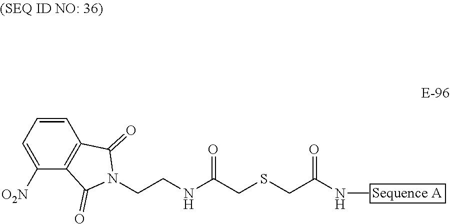 [SMILES: CNC(=O)CSCC(=O)NCCN1C(=O)c2cccc([N+](=O)[O-])c2C1=O]

Peptide E-96 was prepared from 34 mg of resin (approximate loading 0.18 mmol/g) using I-113 and GP5 to afford 4.4 mg of E-96 as a white solid. ESI-MS found 1084.2, C₁₉₆H₂₈₅N₄₇O₆₃S (M+4H⁺) requires 1084.2.

### Example 97: Compound 86

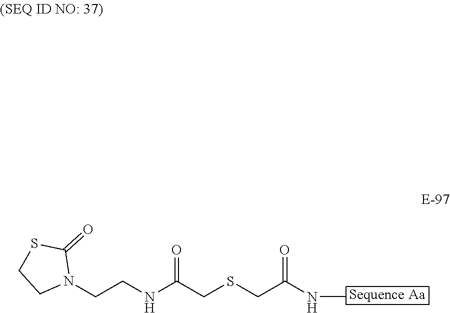 [SMILES: CNC(=O)CSCC(=O)NCCN1CCSC1=O]

Peptide E-97 was prepared from 35 mg of resin (approximate loading 0.125 mmol/g) using I-115 and GP4 to afford 2.5 mg of E-97 as a white solid. ESI-MS found 1154.4, C₂₁₂H₃₂₃N₄₇O₆₄S₂ (M+4H⁺) requires 1153.8.

### Example 98: Compound 146

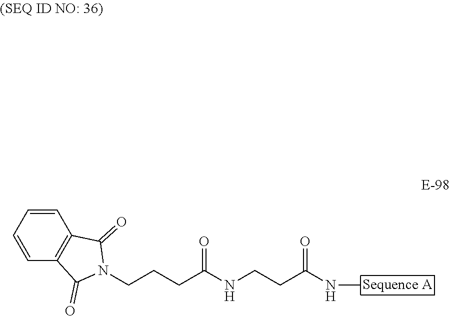 [SMILES: CNC(=O)CCNC(=O)CCCN1C(=O)c2ccccc2C1=O]

Peptide E-98 was prepared from 26 mg of resin (approximate loading 0.18 mmol/g) using I-118 and GP4 to afford E-98 as a white solid. ESI-MS found 1167.0, C₁₉₇H₂₈₂N₄₆O₅₉S (M−4H)⁻ requires 1167.0.

### Example 99: Compound 115

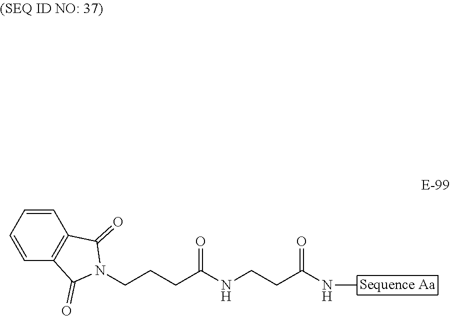 [SMILES: CNC(=O)CCNC(=O)CCCN1C(=O)c2ccccc2C1=O]

Peptide E-99 was prepared from 108 mg of resin (approximate loading 0.18 mmol/g) using I-118 and GP4 to afford 1.0 mg of E-99 as a white solid. ESI-MS found 1160.3, C₂₁₈H₃₂₅N₄₇O₆₅ (M+4H⁺) requires 1160.3.

### Example 100: Compound 154

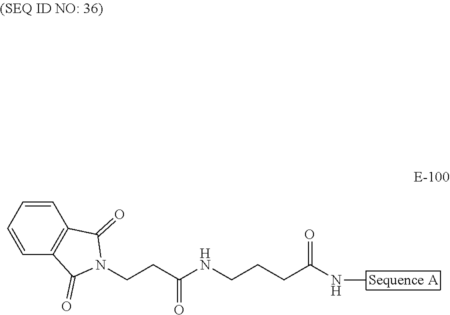 [SMILES: CNC(=O)CCCNC(=O)CCN1C(=O)c2ccccc2C1=O]

To 26 mg of resin (approximate loading 0.18 mmol/g, 4.68 μmol) in a 3 mL polypropylene tube with an end-cap was added 20% v/v piperidine/DMF (2 mL). The tube was capped, agitated at ambient temperature for 30 minutes, and then drained. The resin was washed with DMF (5×3 mL). A solution of carboxylic acid building block I-120 (8 mg, 5.6 equiv.) in DMF (2.8 mL) was added to the resin, followed by EDC (27.4 mg, 30.5 equiv.) and 1-hydroxybenzotriazole hydrate (34 mg, 43.1 equiv.). The reaction mixture was agitated at ambient temperature for 18 hours. The reaction mixture was drained; the resin was washed with DMF (5×3 mL), DCM (5×3 mL), and dried in vacuo for 30 minutes.

The resin was transferred to a 15 mL Falcon tube and 3 mL of cleavage reagent (95:2.5:2.5 v/v/v TFA/TIS/H₂O) was added. The reaction mixture was agitated at ambient temperature for 1 hour. The resin was filtered and washed with TFA (2×3 mL). The combined filtrate and washes were concentrated under reduced pressure to afford a residue, which was triturated with Et₂O (3 mL) to precipitate the peptide. The peptide was re-dissolved in glacial AcOH (2 mL) and purified by preparative HPLC (Phenomenex Jupiter 10 μM Proteo 90 Å LC column, 250×21.2 mm, with flow rate-15 mL/min, gradient of 0-100% acetonitrile in 25 mM aqueous ammonium acetate over 30 minutes) to afford E-100 as a white solid. ESI-MS found 1169.0, C₁₉₇H₂₈₈N₄₆O₆₁ (M+4H⁺) requires 1168.5.

### Example 101: Compound 156

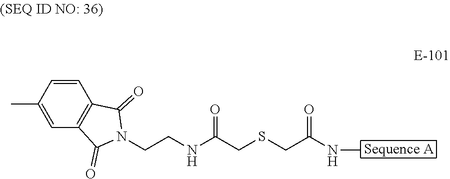

Peptide E-101 was prepared from 25 mg of resin (approximate loading 0.18 mmol/g) using I-122 and GP5 to afford E-101 as a white solid.

### Example 102: Compound 157

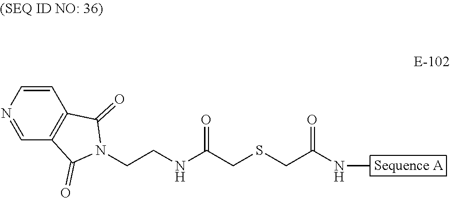 [SMILES: CNC(=O)CSCC(=O)NCCN1C(=O)c2ccncc2C1=O]

Peptide E-102 was prepared from 25 mg of resin (approximate loading 0.18 mmol/g) using I-124 and GP5 to afford E-102 as a white solid. ESI-MS found 1073.8, C₁₉₅H₂₈₅N₄₇O₆₁S (M+4H⁺) requires 1073.3.

### Example 103: Compound 158

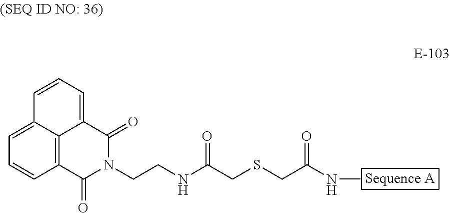 [SMILES: CNC(=O)CSCC(=O)NCCN1C(=O)c2cccc3cccc(c23)C1=O]

Peptide E-103 was prepared from 25 mg of resin (approximate loading 0.18 mmol/g) using I-126 and GP5 to afford E-103 as a white solid. ESI-MS found 1083.5, C₂₀₀H₂₈₀N₄₆O₆₁S (M−4H)⁻ requires 1084.0.

### Example 104: Compound 159

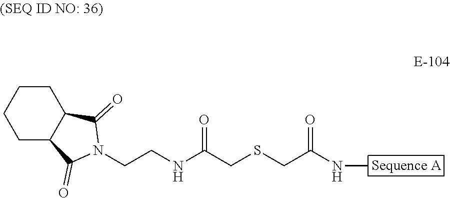 [SMILES: CNC(=O)CSCC(=O)NCCN1C(=O)[C@H]2CCCC[C@H]2C1=O]

Peptide E-104 was prepared from 25 mg of resin (approximate loading 0.18 mmol/g) using I-129 and GP5 to afford E-104 as a white solid. ESI-MS found 1073.0, C₁₉₆H₂₈₄N₄₆O₆₁S (M−4H)⁻ requires 1072.5.

### Example 105: Compound 160

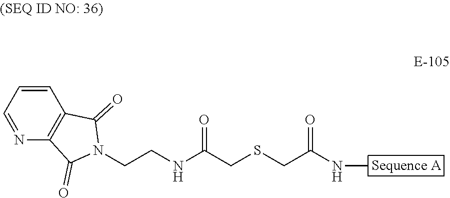 [SMILES: CNC(=O)CSCC(=O)NCCN1C(=O)c2cccnc2C1=O]

Peptide E-105 was prepared from 25 mg of resin (approximate loading 0.18 mmol/g) using I-131 and GP5 to afford E-105 as a white solid. ESI-MS found 1071.8, C₁₉₅H₂₇₇N₄₇O₆₁S (M−4H)⁻ requires 1071.3.

### Example 106: Compound 161

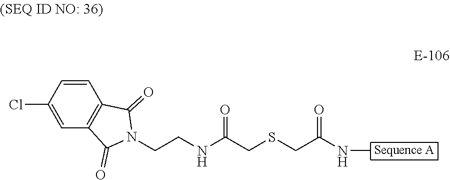 [SMILES: CNC(=O)CSCC(=O)NCCN1C(=O)c2ccc(Cl)cc2C1=O]

Peptide E-106 was prepared from 25 mg of resin (approximate loading 0.18 mmol/g) using I-133 and GP5 to afford E-106 as a white solid. ESI-MS found 1080.2, C₁₉₆H₂₇₇N₄₆O₆₁SCl (M−4H)⁻ requires 1079.5.

### Example 107: Compound 162

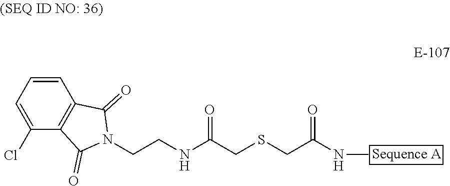 [SMILES: CNC(=O)CSCC(=O)NCCN1C(=O)c2cccc(Cl)c2C1=O]

Peptide E-107 was prepared from 25 mg of resin (approximate loading 0.18 mmol/g) using I-135 and GP5 to afford E-107 as a white solid. ESI-MS found 1080.2, C₁₉₆H₂₇₇N₄₆O₆₁SCl (M−4H)⁻ requires 1079.5.

### Example 108: Compound 163

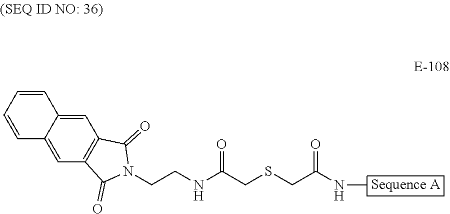 [SMILES: CNC(=O)CSCC(=O)NCCN1C(=O)c2cc3ccccc3cc2C1=O]

Peptide E-108 was prepared from 25 mg of resin (approximate loading 0.18 mmol/g) using I-137 and GP5 to afford E-108 as a white solid. ESI-MS found 1084.0, C₂₀₀H₂₈₀N₄₆O₆₁S (M−4H)⁻ requires 1083.5.

### Example 109: Compound 164

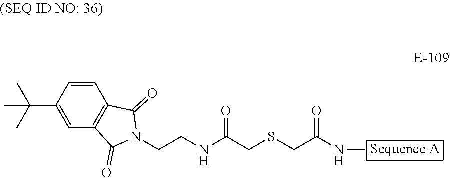 [SMILES: CNC(=O)CSCC(=O)NCCN1C(=O)c2ccc(C(C)(C)C)cc2C1=O]

Peptide E-109 was prepared from 25 mg of resin (approximate loading 0.18 mmol/g) using I-139 and GP5 to afford E-109 as a white solid. ESI-MS found 1085.5, C₂₀₀H₂₉₀N₄₆O₆₁S (M−4H)⁻ requires 1085.0.

### Example 110: Compound 168

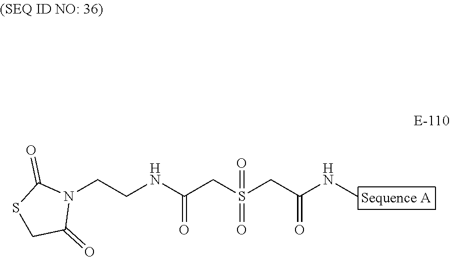 [SMILES: CNC(=O)CS(=O)(=O)CC(=O)NCCN1C(=O)CSC1=O]

E-110 was prepared according to GP4 using 21.8 mg of resin (estimated loading 0.15 mmol/g) and I-141 to afford 2.0 mg of E-110 as a white solid. ESI-MS found 1072.0, C₁₉₁H₂₇₆N₄₆O₆₃S₂ (M−4H)⁻ requires 1071.5.

### Example 111: Compound 10

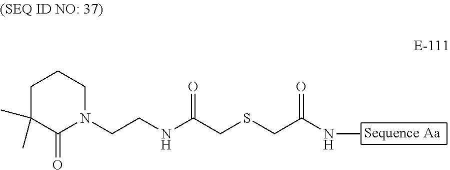

Peptide E-111 was prepared from 30 mg of resin (approximate loading 0.125 mmol/g) using I-145 and GP4 to afford 2.2 mg of E-111 as a white solid. ESI-MS found 1160.3, C₂₁₆H₃₃₁N₄₇O₆₄S (M+4H⁺) requires 1159.8.

### Example 112: Compound 16

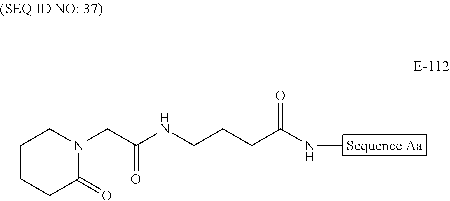 [SMILES: CNC(=O)CCCNC(=O)CN1CCCCC1=O]

Peptide E-112 was prepared from 33 mg of resin (approximate loading 0.125 mmol/g) using I-146 and GP4 to afford 3.7 mg of E-112 as a white solid. ESI-MS found 1145.3, C₂₁₅H₃₂₉N₄₇O₆₄ (M+4H⁺) requires 1144.8.

### Example 113: Compound 15

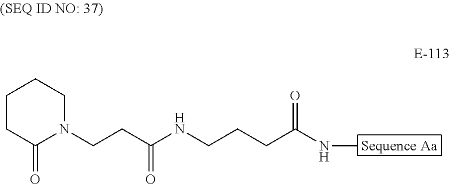 [SMILES: CNC(=O)CCCNC(=O)CCN1CCCCC1=O]

Peptide E-113 was prepared from 33 mg of resin (approximate loading 0.125 mmol/g) using I-147 and GP4 to afford 3.4 mg of E-113 as a white solid. ESI-MS found 1148.8, C₂₁₅H₃₂₉N₄₇O₆₄ (M+4H⁺) requires 1148.3.

### Example 114: Compound 20

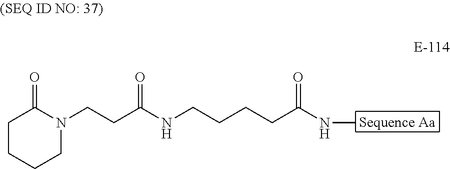 [SMILES: CNC(=O)CCCCNC(=O)CCN1CCCCC1=O]

Peptide E-114 was prepared from 45 mg of resin (approximate loading 0.125 mmol/g) using I-148 and GP4 to afford 5.8 mg of E-114 as a white solid. ESI-MS found 1152.3, C₂₁₆H₃₃₁N₄₇O₆₄ (M+4H⁺) requires 1151.8.

### Example 115: Compound 82

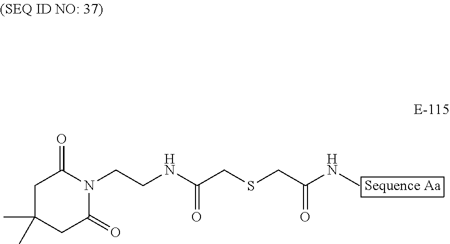

E-115 was prepared according to GP4 using 45.9 mg of resin (estimated loading 0.125 mmol/g) and I-150 to afford 6.0 mg of E-115 as a white solid. ESI-MS found 1163.9, C₂₁₆H₃₂₉N₄₇O₆₅S (M+4H⁺) requires 1163.3.

### Example 116: Compound 95

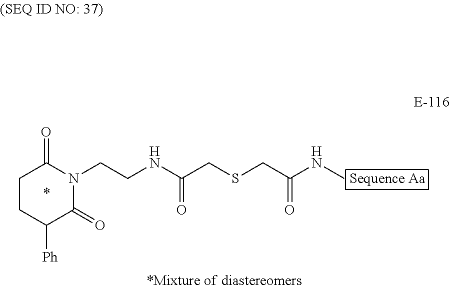 [SMILES: CNC(=O)CSCC(=O)NCCN1C(=O)CCC(c2ccccc2)C1=O]

E-116 was prepared according to GP4 using 30.5 mg of resin (estimated loading 0.125 mmol/g) and I-152 to afford 2.4 mg of E-116 as a white solid. ESI-MS found 1175.9, C₂₂₀H₃₂₉N₄₇O₆₅S (M+4H⁺) requires 1175.3.

### Example 117: Compound 96

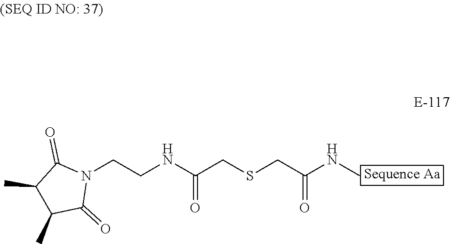

E-117 was prepared according to GP4 using 34.2 mg of resin (estimated loading 0.125 mmol/g) and I-155 to afford 2.1 mg of E-117 as a white solid. ESI-MS found 1160.3, C₂₁₅H₃₂₇N₄₇O₆₅S (M+4H⁺) requires 1159.8.

### Example 118: Compound 100

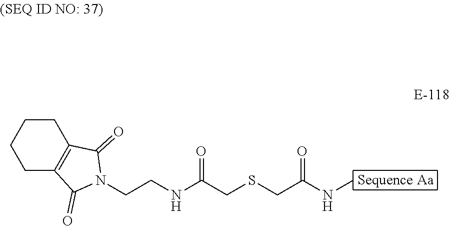 [SMILES: CNC(=O)CSCC(=O)NCCN1C(=O)C2=C(CCCC2)C1=O]

E-118 was prepared according to GP4 using 28 mg of resin (estimated loading 0.125 mmol/g) and I-157 to afford E-118 as a white solid. ESI-MS found 1166.4, C₂₁₇H₃₂₇N₄₇O₆₅S (M+4H⁺) requires 1165.8.

### Example 119: Compound 101

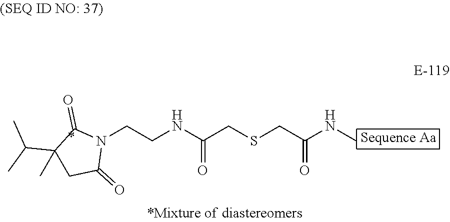 [SMILES: CNC(=O)CSCC(=O)NCCN1C(=O)CC(C)(C(C)C)C1=O]

E-119 was prepared according to GP4 using 29.8 mg of resin (estimated loading 0.125 mmol/g) and I-159 to afford 3.6 mg of E-119 as a white solid. ESI-MS found 1167.1, C₂₁₇H₃₃₁N₄₇O₆₅S (M+4H⁺) requires 1166.8.

### Example 120: Compound 102

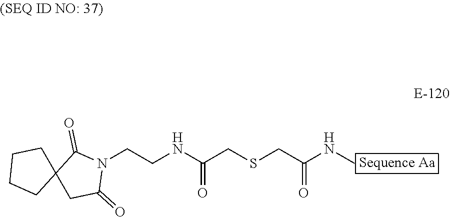 [SMILES: CNC(=O)CSCC(=O)NCCN1C(=O)CC2(CCCC2)C1=O]

E-120 was prepared according to GP4 using 29.3 mg of resin (estimated loading 0.125 mmol/g) and I-161 to afford 4.4 mg of E-120 as a white solid. ESI-MS found 1166.8, C₂₁₇H₃₂₉N₄₇O₆₅S (M+4H⁺) requires 1166.3.

### Example 121: Compound 104

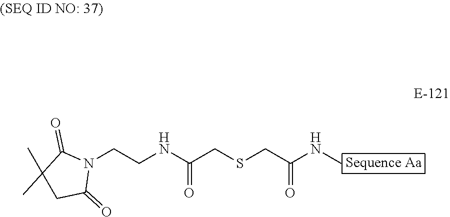

E-121 was prepared according to GP4 using 26.2 mg of resin (estimated loading 0.125 mmol/g) and I-163 to afford 3.2 mg of E-121 as a white solid. ESI-MS found 1160.3, C₂₁₅H₃₂₇N₄₇O₆₅S (M+4H⁺) requires 1159.8.

### Example 122: Compound 110

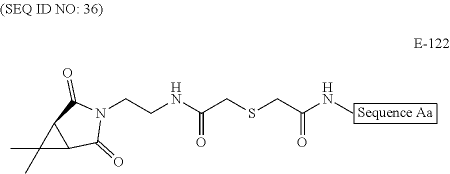

E-122 was prepared according to GP4 using 29.3 mg of resin (estimated loading 0.18 mmol/g) and I-165 to afford 0.6 mg of E-122 as a white solid. ESI-MS found 1070.9, C₁₉₅H₂₉₀N₄₆O₆₁S (M+4H⁺) requires 1071.0.

### Example 123: Compound 111

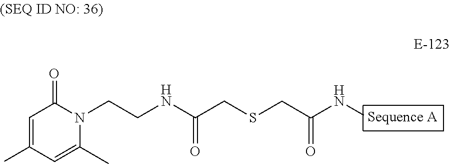

E-123 was prepared according to GP4 using 28.9 mg of resin (estimated loading 0.18 mmol/g) and I-167 to afford 4.1 mg of E-123 as a white solid. ESI-MS found 1067.1, C₁₉₅H₂₉₀N₄₆O₆₀S (M+4H⁺) requires 1067.0.

### Example 124: Compound 26

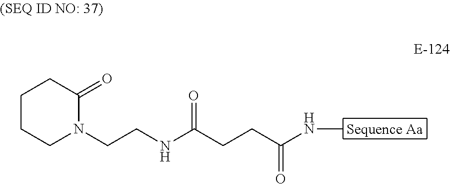 [SMILES: CNC(=O)CCC(=O)NCCN1CCCCC1=O]

E-124 was prepared according to GP4 using 42.9 mg of resin (estimated loading 0.125 mmol/g) and I-168 to afford 18.6 mg of E-124 as a white solid. ESI-MS found 1145.4, C₂₁₄H₃₂₇N₄₇O₆₄ (M+4H⁺) requires 1144.8.

### Example 125: Compound 27

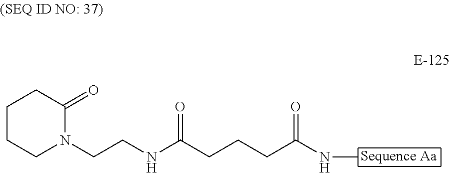 [SMILES: CNC(=O)CCCC(=O)NCCN1CCCCC1=O]

E-125 was prepared according to GP4 using 45.8 mg of resin (estimated loading 0.125 mmol/g) and I-169 to afford 5.4 mg of I-169 as a white solid. ESI-MS found 1148.9, C₂₁₅H₃₂₉N₄₇O₆₄ (M+4H⁺) requires 1148.3.

### Example 126: Compound 18

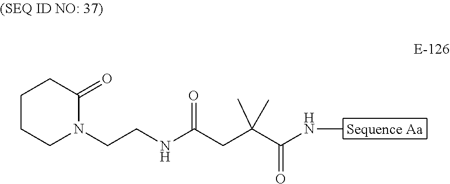 [SMILES: CNC(=O)C(C)(C)CC(=O)NCCN1CCCCC1=O]

E-126 was prepared according to GP4 using 45.5 mg of resin (estimated loading 0.125 mmol/g) and I-170 to afford 4.7 mg of E-126 as a white solid. ESI-MS found 1152.3, C₂₁₆H₃₃₁N₄₇O₆₄ (M+4H⁺) requires 1151.8.

### Example 127: Compound 112

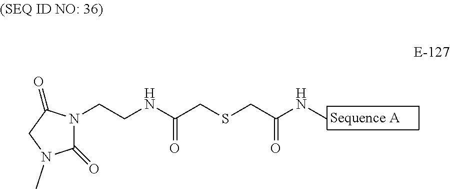 [SMILES: CNC(=O)CSCC(=O)NCCN1C(=O)CN(C)C1=O]

E-127 was prepared according to GP4 using 36.2 mg of resin (estimated loading 0.18 mmol/g) and I-172 to afford 2.1 mg of E-127 as a white solid. ESI-MS found 1064.8, C₁₉₂H₂₈₇N₄₇O₆₁S (M+4H⁺) requires 1064.8.

### Example 128: Compound 9

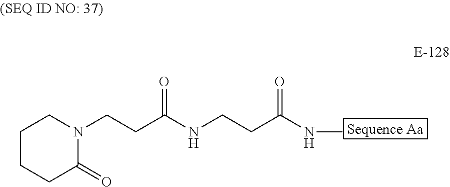 [SMILES: CNC(=O)CCNC(=O)CCN1CCCCC1=O]

E-128 was prepared according to GP4 using 38.3 mg of resin (estimated loading 0.125 mmol/g) and I-174 to afford 3.1 mg of E-128 as a white solid. ESI-MS found 1145.4, C₂₁₄H₃₂₇N₄₇O₆₄ (M+4H⁺) requires 1144.8.

### Example 129: Compound 5

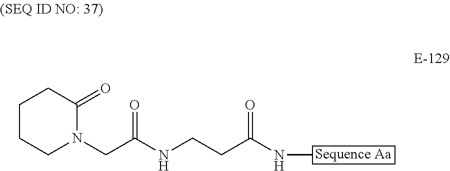 [SMILES: CNC(=O)CCNC(=O)CN1CCCCC1=O]

E-129 was prepared according to GP4 using 40.1 mg of resin (estimated loading 0.125 mmol/g) and I-176 to afford 5.2 mg of E-129 as a white solid. ESI-MS found 1141.8, C₂₁₃H₃₂₅N₄₇O₆₄ (M+4H⁺) requires 1141.3.

### Example 130: Compound 4

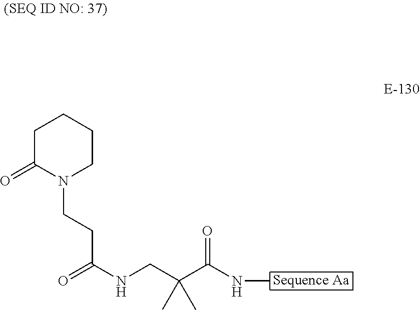 [SMILES: CNC(=O)C(C)(C)CNC(=O)CCN1CCCCC1=O]

E-130 was prepared according to GP4 using 40 mg of resin (estimated loading 0.125 mmol/g) and I-180 to afford 5.2 mg of E-130 as a white solid. ESI-MS found 1152.4, C₂₁₆H₃₃₁N₄₇O₆₄ (M+4H⁺) requires 1151.8.

### Example 131: Compound 3

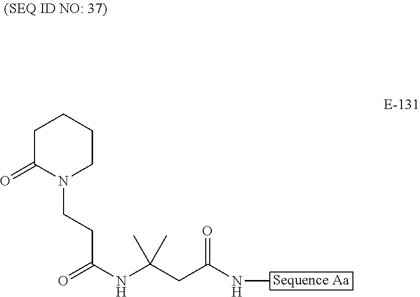 [SMILES: CNC(=O)CC(C)(C)NC(=O)CCN1CCCCC1=O]

E-131 was prepared according to GP4 using 39.3 mg of resin (estimated loading 0.125 mmol/g) and I-183 to afford 3.0 mg of E-131 as a white solid. ESI-MS found 1152.3, C₂₁₆H₃₃₁N₄₇O₆₄ (M+4H⁺) requires 1151.8.

### Example 132: Compound 2

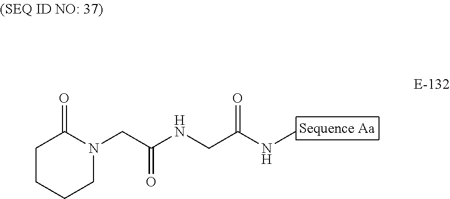 [SMILES: CNC(=O)CNC(=O)CN1CCCCC1=O]

E-132 was prepared according to GP4 using 39.8 mg of resin (estimated loading 0.125 mmol/g) and I-184 to afford 5.7 mg of E-132 as a white solid. ESI-MS found 1138.3, C₂₁₂H₃₂₃N₄₇O₆₄ (M+4H⁺) requires 1141.3.

### Example 133: Compound 1

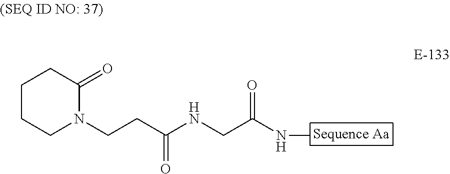 [SMILES: CNC(=O)CNC(=O)CCN1CCCCC1=O]

E-133 was prepared according to GP4 using 38.5 mg of resin (estimated loading 0.125 mmol/g) and I-185 to afford 4.3 mg of E-133 as a white solid. ESI-MS found 1141.8, C₂₁₃H₃₂₅N₄₇O₆₄ (M+4H⁺) requires 1141.3.

### Example 134: Compound 130

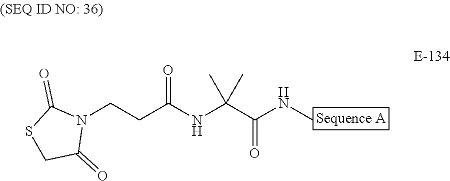 [SMILES: CNC(=O)C(C)(C)NC(=O)CCN1C(=O)CSC1=O]

E-134 was prepared according to GP4 using 27 mg of resin (estimated loading 0.18 mmol/g) and I-187 to afford 1.6 mg of E-134 as a white solid. ESI-MS found 1059.5, C₁₉₂H₂₇₈N₄₆O₆₁S (M−4H)⁻ requires 1059.0.

### Example 135: Compound 167

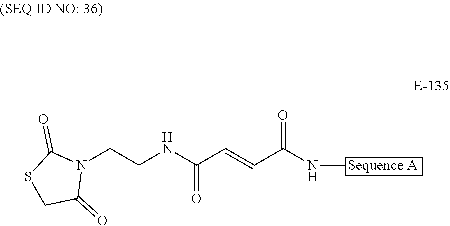 [SMILES: CNC(=O)/C=C/C(=O)NCCN1C(=O)CSC1=O]

E-135 was prepared according to GP4 using 25.1 mg of resin (estimated loading 0.15 mmol/g) and I-188 to afford 3.7 mg of E-135 as a white solid. ESI-MS found 1055.5, C₁₉₁H₂₇₄N₄₆O₆₁S (M−4H)⁻ requires 1055.0.

### Example 136: Compound 179

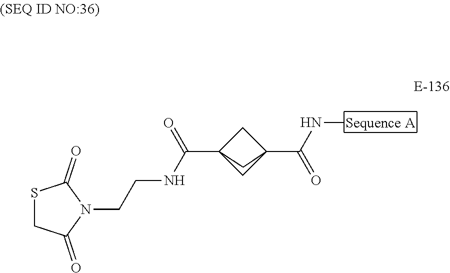

E-136 was prepared according to GP5 using 20.9 mg of resin (estimated loading 0.15 mmol/g) and I-191 to afford 1.6 mg of E-136 as a white solid. ESI-MS found 1065.5, C₁₉₄H₂₇₈N₄₆O₆₁S (M−4H)⁻ requires 1065.0.

### Example 137: Compound 183

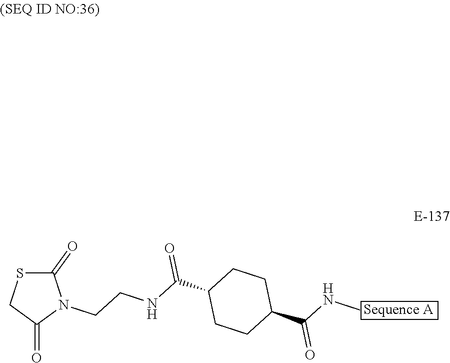 [SMILES: CNC(=O)[C@H]1CC[C@H](C(=O)NCCN2C(=O)CSC2=O)CC1]

E-137 was prepared according to GP5 using 16.6 mg of resin (estimated loading 0.5 mmol/g) and I-194 to afford 1.3 mg of E-137 as a white solid. ESI-MS found 1069.5, C₁₉₅H₂₈₂N₄₆O₆₁S (M−4H)⁻ requires 1069.0.

### Example 138: Compound 182

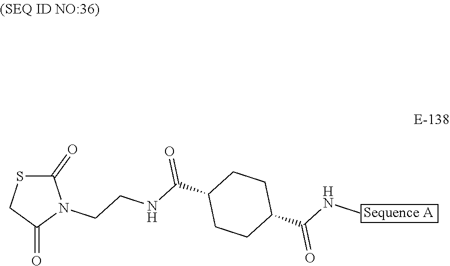 [SMILES: CNC(=O)[C@H]1CC[C@@H](C(=O)NCCN2C(=O)CSC2=O)CC1]

E-138 was prepared according to GP5 using 16.9 mg of resin (estimated loading 0.5 mmol/g) and I-197 to afford 1.0 mg of E-138 as a white solid. ESI-MS found 1069.5, C₁₉₅H₂₈₂N₄₆O₆₁S (M−4H)⁻ requires 1069.0.

### Example 139: Compound 176

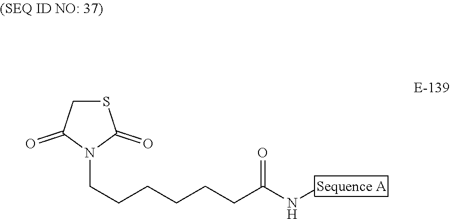

E-139 was prepared according to GP4 using 22.2 mg of resin (estimated loading 0.15 mmol/g) and I-200 to afford 0.9 mg of E-139 as a white solid. ESI-MS found 1052.3, C₁₉₂H₂₇₉N₄₅O₆₀S (M−4H)⁻ requires 1051.8.

### Example 140: Compound 175

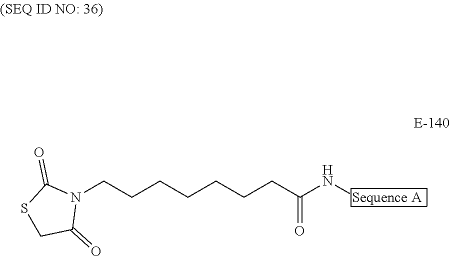

E-140 was prepared according to GP4 using 31 mg of resin (estimated loading 0.15 mmol/g) and I-202 to afford 2.7 mg of E-140 as a white solid. ESI-MS found 1055.8, C₁₉₃H₂₈₁N₄₅O₆₀S (M−4H)⁻ requires 1055.3.

### Example 141: Compound 174

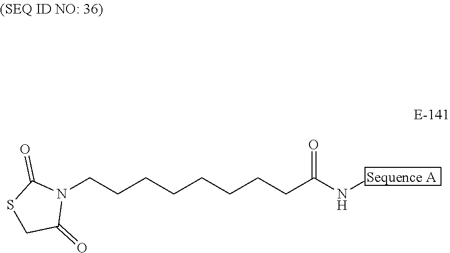

E-141 was prepared according to GP4 using 24.7 mg of resin (estimated loading 0.15 mmol/g) and I-204 to afford 1.6 mg of E-141 as a white solid. ESI-MS found 1059.3, C₁₉₄H₂₈₃N₄₅O₆₀S (M−4H)⁻ requires 1058.8.

### Example 142: Compound 166

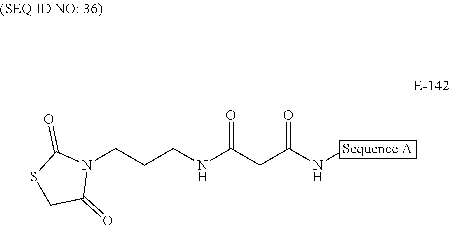

E-142 was prepared according to GP4 using 20.7 mg of resin (estimated loading 0.15 mmol/g) and I-209 to afford 2.4 mg of E-142 as a white solid. ESI-MS found 1056.0, C₁₉₁H₂₇₄N₄₆O₆₁S (M−4H)⁻ requires 1055.5.

### Example 143: Compound 165

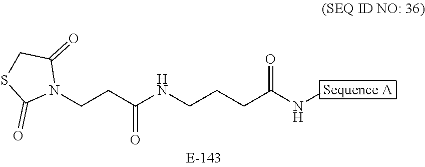

E-143 was prepared according to GP4 using 21 mg of resin (estimated loading 0.15 mmol/g) and I-213 to afford 2.1 mg of E-143 as a white solid. ESI-MS found 1059.5, C₁₉₂H₂₇₈N₄₆O₆₁S (M−4H)⁻ requires 1059.0.

### Example 144: Compound 51

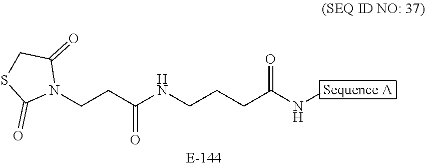

E-144 was prepared according to GP4 using 45.5 mg of resin (estimated loading 0.125 mmol/g) and I-213 to afford 4.8 mg of E-144 as a white solid. ESI-MS found 1153.1, C₂₁₃H₃₂₃N₄₇O₆₅S (M+4H⁺) requires 1152.8.

### Example 145: Compound 172

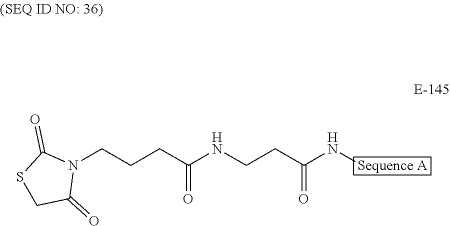

E-145 was prepared according to GP4 using 23 mg of resin (estimated loading 0.15 mmol/g) and I-217 to afford 3.0 mg of E-145 as a white solid. ESI-MS found 1059.5, C₁₉₂H₂₇₈N₄₆O₆₁S (M−4H)⁻ requires 1059.0.

### Example 146: Compound 171

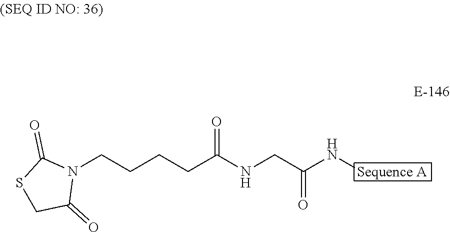

E-146 was prepared according to GP4 using 24.1 mg of resin (estimated loading 0.15 mmol/g) and I-221 to afford 2.5 mg of E-146 as a white solid. ESI-MS found 1059.5, C₁₉₂H₂₇₈N₄₆O₆₁S (M−4H)⁻ requires 1059.0.

### Example 147: Compound 48

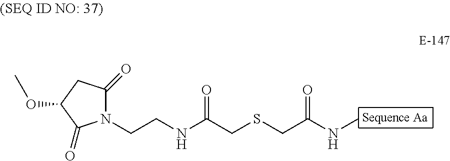

E-147 was prepared according to GP4 using 42.5 mg of resin (estimated loading 0.125 mmol/g) and I-225 to afford 1.7 mg of E-147 as a white solid. ESI-MS found 1160.8, C₂₁₄H₃₂₅N₄₇O₆₆S (M+4H⁺) requires 1160.3.

### Example 148: Compound 49

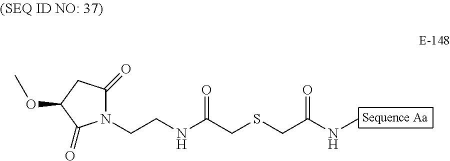

E-148 was prepared according to GP4 using 44.5 mg of resin (estimated loading 0.125 mmol/g) and I-229 to afford 2.8 mg of E-148 as a white solid. ESI-MS found 1160.7, C₂₁₄H₃₂₅N₄₇O₆₆S (M+4H⁺) requires 1160.3.

### Example 149: Compound 28

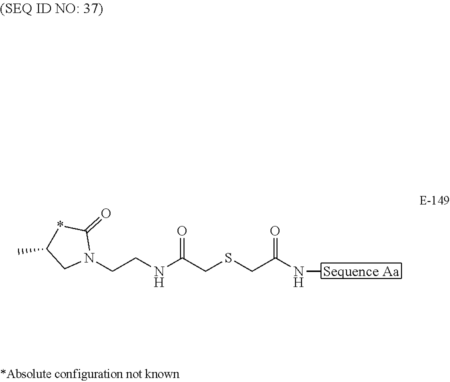

E-149 was prepared according to GP4 using 38.9 mg of resin (estimated loading 0.125 mmol/g) and (R*)-I-234 to afford 5.7 mg of E-149 as a white solid. ESI-MS found 1153.3, C₂₁₄H₃₂₇N₄₇O₆₄S (M+4H⁺) requires 1152.8.

### Example 150: Compound 64

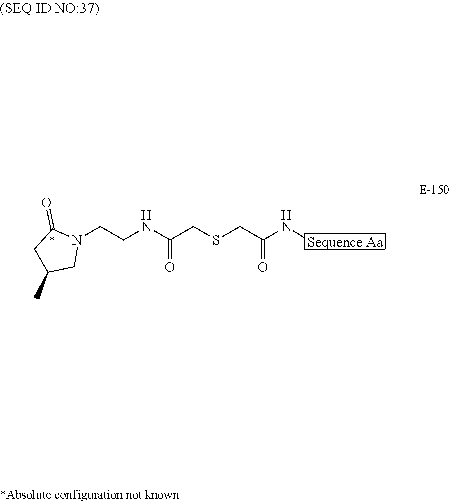

E-150 was prepared according to GP4 using 39 mg of resin (estimated loading 0.125 mmol/g) and (S*)-I-234 to afford 4.2 mg of E-190 as a white solid. ESI-MS found 1153.3, C₂₁₄H₃₂₇N₄₇O₆₄S (M+4H⁺) requires 1152.8.

### Example 151: Compound 55

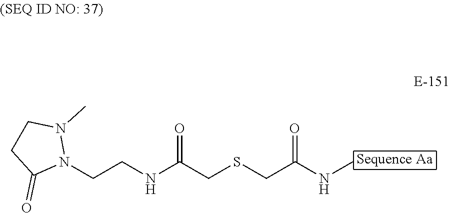 [SMILES: CNC(=O)CSCC(=O)NCCN1C(=O)CCN1C]

E-151 was prepared according to GP4 using 43.4 mg of resin (estimated loading 0.125 mmol/g) and I-240 to afford 6.0 mg of E-151 as a white solid. ESI-MS found 1153.6, C₂₁₃H₃₂₆N₄₈O₆₄S (M+4H⁺) requires 1153.1.

### Example 152: Compound 58

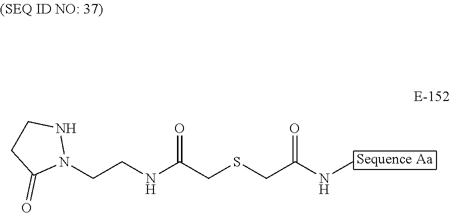 [SMILES: CNC(=O)CSCC(=O)NCCN1NCCC1=O]

E-152 was prepared according to GP4 using 37.7 mg of resin (estimated loading 0.125 mmol/g) and I-243 to afford 1.8 mg of E-152 as a white solid. ESI-MS found 1150.0, C₂₁₂H₃₂₄N₄₈O₆₄S (M+4H⁺) requires 1149.6.

### Example 153: Compound 65

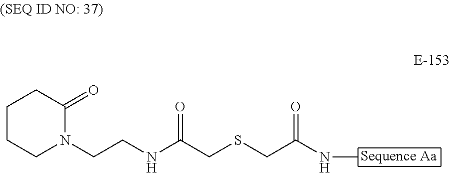 [SMILES: CNC(=O)CSCC(=O)NCCN1CCCCC1=O]

E-153 was prepared according to GP4 using 41 mg of resin (estimated loading 0.125 mmol/g) and I-248 to afford 4.1 mg of E-153 as a white solid. ESI-MS found 1153.3, C₂₁₄H₃₂₇N₄₇O₆₄S (M+4H⁺) requires 1152.8.

### Example 154: Compound 57

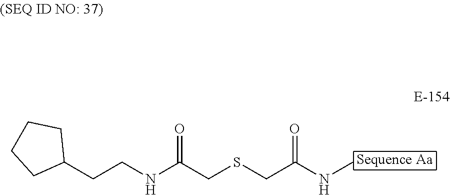 [SMILES: CNC(=O)CSCC(=O)NCCC1CCCC1]

E-154 was prepared according to GP4 using 41.1 mg of resin (estimated loading 0.125 mmol/g) and I-249 to afford 6.5 mg of E-154 as a white solid. ESI-MS found 1146.1, C₂₁₄H₃₂₈N₄₆O₆₃S (M+4H⁺) requires 1145.6.

### Example 155: Compound 54

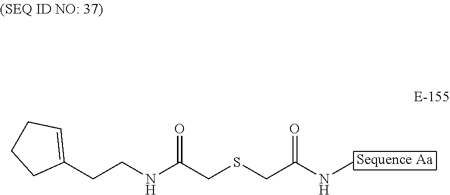 [SMILES: CNC(=O)CSCC(=O)NCCC1=CCCC1]

E-155 was prepared according to GP4 using 39.8 mg of resin (estimated loading 0.125 mmol/g) and I-250 to afford 4.7 mg of E-155 as a white solid. ESI-MS found 1145.6, C₂₁₄H₃₂₆N₄₆O₆₃S (M+4H⁺) requires 1145.

### Example 156: Compound 50

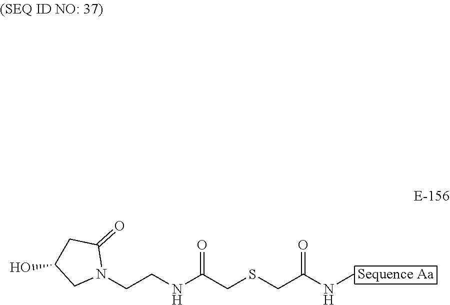 [SMILES: CNC(=O)CSCC(=O)NCCN1C[C@H](O)CC1=O]

E-156 was prepared according to GP4 using 46.9 mg of resin (estimated loading 0.125 mmol/g) and I-257 to afford 2.7 mg of E-156 as a white solid. ESI-MS found 1153.8, C₂₁₃H₃₂₅N₄₇O₆₅S (M+4H⁺) requires 1153.3.

### Example 157: Compound 45

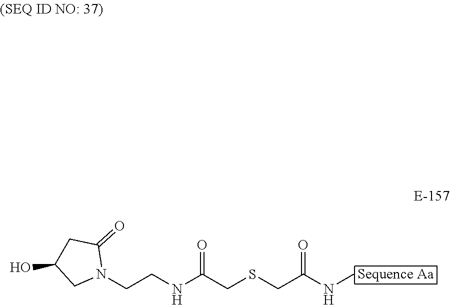 [SMILES: CNC(=O)CSCC(=O)NCCN1C[C@@H](O)CC1=O]

E-157 was prepared according to GP4 using 45.5 mg of resin (estimated loading 0.125 mmol/g) and I-264 to afford 7.0 mg of E-157 as a white solid. ESI-MS found 1153.7, C₂₁₃H₃₂₅N₄₇O₆₅S (M+4H⁺) requires 1153.3.

### Example 158: Compound 43

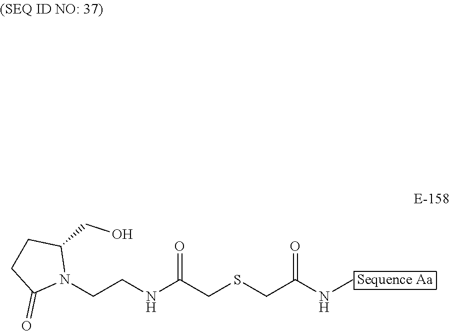 [SMILES: CNC(=O)CSCC(=O)NCCN1C(=O)CC[C@@H]1CO]

E-158 was prepared according to GP4 using 44.5 mg of resin (estimated loading 0.125 mmol/g) and I-271 to afford 2.7 mg of E-158 as a white solid. ESI-MS found 1157.2, C₂₁₄H₃₂₇N₄₇O₆₅S (M+4H⁺) requires 1156.8.

### Example 159: Compound 44

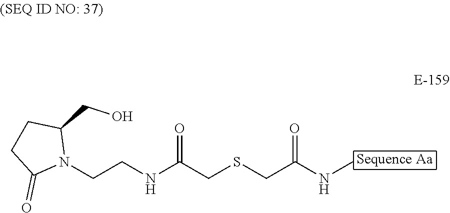 [SMILES: CNC(=O)CSCC(=O)NCCN1C(=O)CC[C@H]1CO]

E-159 was prepared according to GP4 using 39.9 mg of resin (estimated loading 0.125 mmol/g) and I-278 to afford 2.6 mg of E-159 as a white solid. ESI-MS found 1157.3, C₂₁₄H₃₂₇N₄₇O₆₅S (M+4H⁺) requires 1156.8.

### Example 160: Compound 39

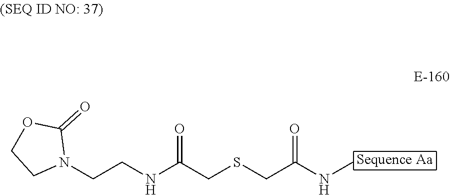 [SMILES: CNC(=O)CSCC(=O)NCCN1CCOC1=O]

E-160 was prepared according to GP4 using 43.8 mg of resin (estimated loading 0.125 mmol/g) and I-279 to afford 5.5 mg of E-160 as a white solid. ESI-MS found 1150.3, C₂₁₂H₃₂₃N₄₇O₆₄S (M+4H⁺) requires 1149.8.

### Example 161: Compound 59

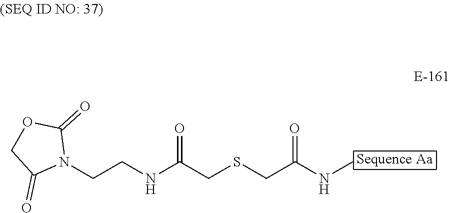 [SMILES: CNC(=O)CSCC(=O)NCCN1C(=O)COC1=O]

E-161 was prepared according to GP4 using 39.7 mg of resin (estimated loading 0.125 mmol/g) and I-282 to afford 4.8 mg of E-161 as a white solid. ESI-MS found 1153.9, C₂₁₂H₃₂₁N₄₇O₆₆S (M+4H⁺) requires 1153.3.

### Example 162: Compound 21

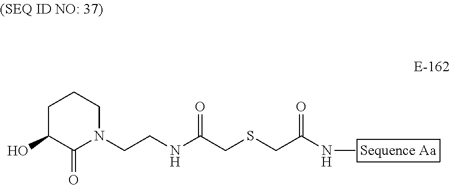 [SMILES: CNC(=O)CSCC(=O)NCCN1CCC[C@H](O)C1=O]

E-162 was prepared according to GP4 using 40.4 mg of resin (estimated loading 0.125 mmol/g) and I-289 to afford 5.3 mg of E-162 as a white solid. ESI-MS found 1157.3, C₂₁₄H₃₂₇N₄₇O₆₅S (M+4H⁺) requires 1156.8.

### Example 163: Compound 7

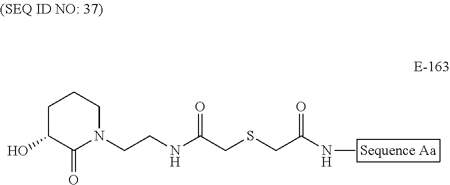 [SMILES: CNC(=O)CSCC(=O)NCCN1CCC[C@@H](O)C1=O]

E-163 was prepared according to GP4 using 36.6 mg of resin (estimated loading 0.125 mmol/g) and I-296 to afford 2.9 mg of E-163 as a white solid. ESI-MS found 1157.4, C₂₁₄H₃₂₇N₄₇O₆₅S (M+4H⁺) requires 1156.8.

### Example 164: Compound 34

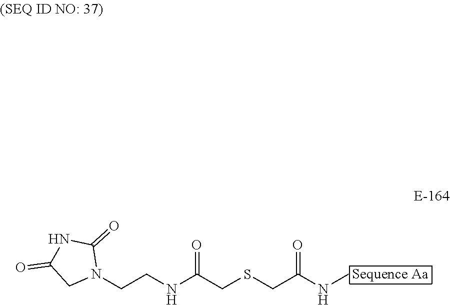 [SMILES: CNC(=O)CSCC(=O)NCCN1CC(=O)NC1=O]

E-164 was prepared according to GP4 using 41.3 mg of resin (estimated loading 0.125 mmol/g) and I-301 to afford 2.0 mg of E-164 as a white solid. ESI-MS found 1153.7, C₂₁₂H₃₂₂N₄₈O₆₅S (M+4H⁺) requires 1153.1.

### Example 165: Compound 46

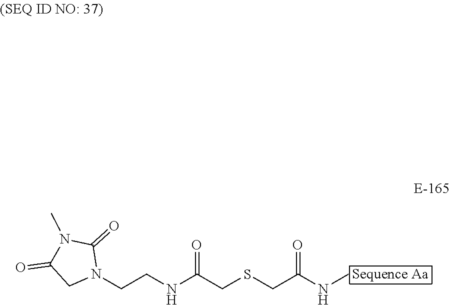 [SMILES: CNC(=O)CSCC(=O)NCCN1CC(=O)N(C)C1=O]

E-165 was prepared according to GP4 using 38.4 mg of resin (estimated loading 0.125 mmol/g) and I-307 to afford 5.3 mg of E-165 as a white solid. ESI-MS found 1157.1, C₂₁₃H₃₂₄N₄₈O₆₅S (M+4H⁺) requires 1156.6.

### Example 166: Compound 25

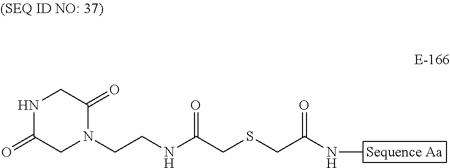 [SMILES: CNC(=O)CSCC(=O)NCCN1CC(=O)NCC1=O]

E-166 was prepared according to GP4 using 34.3 mg of resin (estimated loading 0.125 mmol/g) and I-311 to afford 3.3 mg of E-166 as a white solid. ESI-MS found 1157.1, C₂₁₃H₃₂₄N₄₈O₆₅S (M+4H⁺) requires 1156.6.

### Example 167: Compound 12

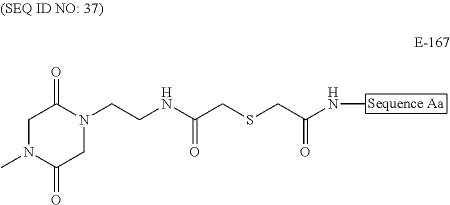

E-167 was prepared according to GP4 using 30 mg of resin (estimated loading 0.125 mmol/g) and I-320 to afford 4.0 mg of E-167 as a white solid. ESI-MS found 1160.6, C₂₁₄H₃₂₆N₄₈O₆₅S (M+4H⁺) requires 1160.1.

### Example 168: Compound 33

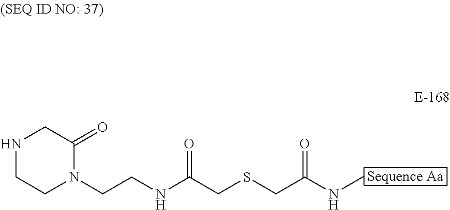 [SMILES: CNC(=O)CSCC(=O)NCCN1CCNCC1=O]

E-168 was prepared according to GP4 using 39.6 mg of resin (estimated loading 0.125 mmol/g) and I-325 to afford 4.9 mg of E-168 as a white solid. ESI-MS found 1153.7, C₂₁₃H₃₂₆N₄₈O₆₄S (M+4H⁺) requires 1153.1.

### Example 169: Compound 24

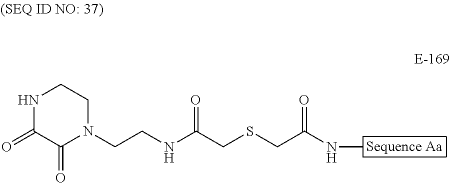 [SMILES: CNC(=O)CSCC(=O)NCCN1CCNC(=O)C1=O]

E-169 was prepared according to GP4 using 39.5 mg of resin (estimated loading 0.125 mmol/g) and I-328 to afford 5.8 mg of E-169 as a white solid. ESI-MS found 1157.1, C₂₁₃H₃₂₄N₄₈O₆₅S (M+4H⁺) requires 1156.6.

### Example 170: Compound 30

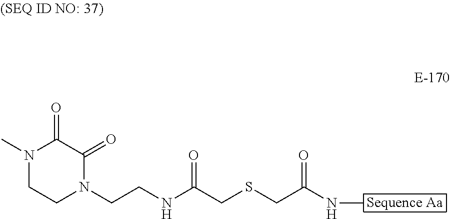

E-170 was prepared according to GP4 using 36.3 mg of resin (estimated loading 0.125 mmol/g) and I-332 to afford 4.4 mg of E-170 as a white solid. ESI-MS found 1160.7, C₂₁₄H₃₂₆N₄₈O₆₅S (M+4H⁺) requires 1160.1.

### Example 171: Compound 38

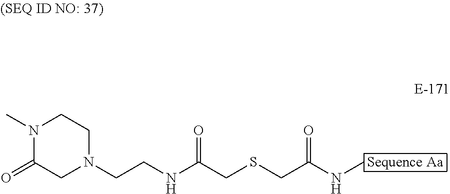

E-171 was prepared according to GP4 using 43.4 mg of resin (estimated loading 0.125 mmol/g) and I-335 to afford 4.3 mg of E-171 as a white solid. ESI-MS found 1157.2, C₂₁₄H₃₂₈N₄₈O₆₄S (M+4H⁺) requires 1156.6.

### Example 172: Compound 37

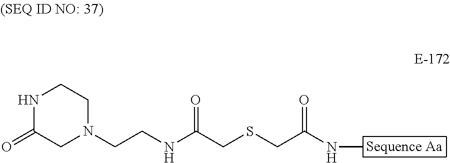 [SMILES: CNC(=O)CSCC(=O)NCCN1CCNC(=O)C1]

E-172 was prepared according to GP4 using 42.5 mg of resin (estimated loading 0.125 mmol/g) and I-338 to afford 4.5 mg of E-172 as a white solid. ESI-MS found 1153.5, C₂₁₃H₃₂₆N₄₈O₆₄S (M+4H⁺) requires 1153.1.

### Example 173: Compound 32

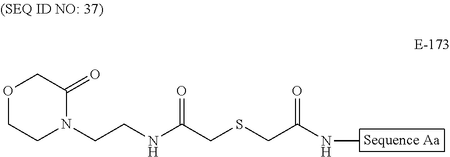 [SMILES: CNC(=O)CSCC(=O)NCCN1CCOCC1=O]

E-173 was prepared according to GP4 using 38.5 mg of resin (estimated loading 0.125 mmol/g) and I-343 to afford 4.5 mg of E-172 as a white solid. ESI-MS found 1153.9, C₂₁₃H₃₂₅N₄₇O₆₅S (M+4H⁺) requires 1153.3.

### Example 174: Compound 36

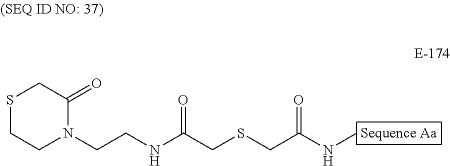 [SMILES: CNC(=O)CSCC(=O)NCCN1CCSCC1=O]

E-174 was prepared according to GP4 using 42.4 mg of resin (estimated loading 0.125 mmol/g) and I-348 to afford 4.5 mg of E-174 as a white solid. ESI-MS found 1157.9, C₂₁₃H₃₂₅N₄₇O₆₄S₂ (M+4H⁺) requires 1157.3.

### Example 175: Compound 29

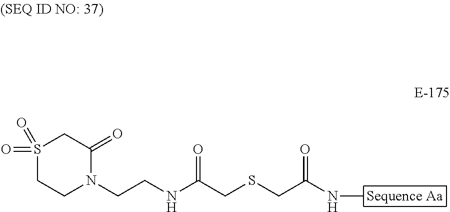 [SMILES: CNC(=O)CSCC(=O)NCCN1CCS(=O)(=O)CC1=O]

E-175 was prepared according to GP4 using 45 mg of resin (estimated loading 0.125 mmol/g) and I-351 to afford 6.5 mg of E-175 as a white solid. ESI-MS found 1165.8, C₂₁₃H₃₂₅N₄₇O₆₆S₂ (M+4H⁺) requires 1165.3.

### Example 176: Compound 6

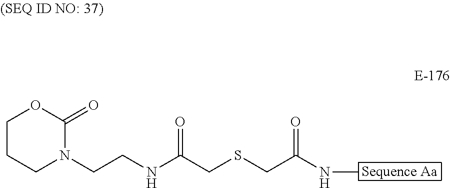 [SMILES: CNC(=O)CSCC(=O)NCCN1CCCOC1=O]

E-176 was prepared according to GP4 using 38.5 mg of resin (estimated loading 0.125 mmol/g) and I-356 to afford 5.1 mg of E-176 as a white solid. ESI-MS found 1153.9, C₂₁₃H₃₂₅N₄₇O₆₅S (M+4H⁺) requires 1153.3.

### Example 177: Compound 23

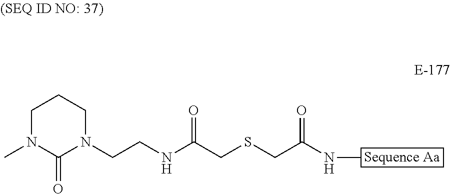

E-177 was prepared according to GP4 using 39 mg of resin (estimated loading 0.125 mmol/g) and I-363 to afford 5.2 mg of E-177 as a white solid. ESI-MS found 1157.2, C₂₁₄H₃₂₈N₄₈O₆₄S (M+4H⁺) requires 1156.6.

### Example 178: Compound 31

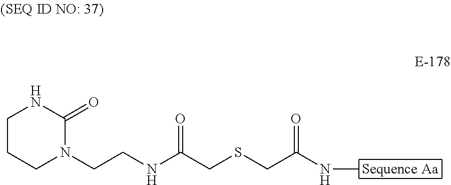 [SMILES: CNC(=O)CSCC(=O)NCCN1CCCNC1=O]

E-178 was prepared according to GP4 using 38.5 mg of resin (estimated loading 0.125 mmol/g) and I-366 to afford 4.8 mg of E-178 as a white solid. ESI-MS found 1153.7, C₂₁₃H₃₂₆N₄₈O₆₄S (M+4H⁺) requires 1153.1.

### Example 179: Compound 11

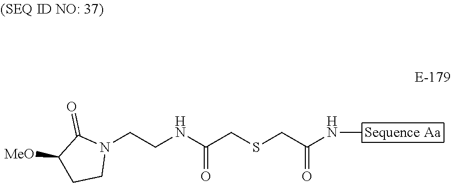 [SMILES: CNC(=O)CSCC(=O)NCCN1CC[C@@H](OC)C1=O]

E-179 was prepared according to GP4 using 30 mg of resin (estimated loading 0.125 mmol/g) and I-373 to afford 4.1 mg of E-179 as a white solid. ESI-MS found 1157.3, C₂₁₄H₃₂₇N₄₇O₆₅S (M+4H⁺) requires 1156.8.

### Example 180: Compound 14

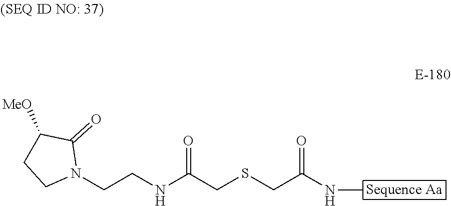 [SMILES: CNC(=O)CSCC(=O)NCCN1CC[C@H](OC)C1=O]

Peptide E-180 was prepared from 30 mg of resin (approximate loading 0.125 mmol/g) using I-380 and GP4 to afford 2.3 mg of E-180 as a white solid. ESI-MS found 1157.4, C₂₁₄H₃₂₇N₄₇O₆₅S (M+4H⁺) requires 1156.8.

### Example 181: Compound 22

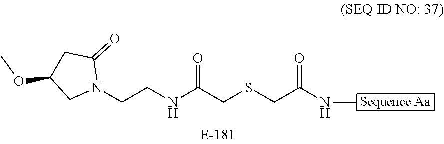

E-181 was prepared according to GP4 using 40.3 mg of resin (estimated loading 0.125 mmol/g) and I-386 to afford 5.5 mg of E-181 as a white solid. ESI-MS found 1157.3, C₂₁₄H₃₂₇N₄₇O₆₅S (M+4H⁺) requires 1156.8.

### Example 182: Compound 8

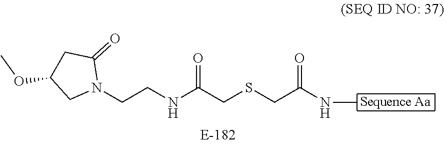

E-182 was prepared according to GP4 using 41 mg of resin (estimated loading 0.125 mmol/g) and I-392 to afford 5.1 mg of E-182 as a white solid. ESI-MS found 1157.3, C₂₁₄H₃₂₇N₄₇O₆₅S (M+4H⁺) requires 1156.8.

### Example 183: Compound 62

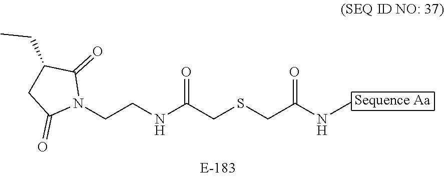 [SMILES: CC[C@H]1CC(=O)N(CCNC(=O)CSCC(=O)NC)C1=O]

E-183 was prepared according to GP4 using 40.8 mg of resin (estimated loading 0.125 mmol/g) and (S*)-I-395 to afford 4.5 mg of E-183 as a white solid. ESI-MS found 1160.3, C₂₁₅H₃₂₇N₄₇O₆₅S (M+4H⁺) requires 1159.8.

### Example 184: Compound 61

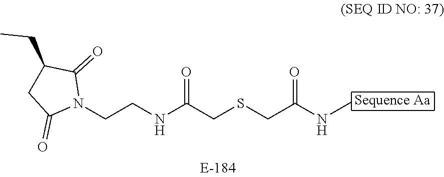 [SMILES: CC[C@@H]1CC(=O)N(CCNC(=O)CSCC(=O)NC)C1=O]

E-184 was prepared according to GP4 using 38 mg of resin (estimated loading 0.125 mmol/g) and (R*)-I-395 to afford 3.0 mg of E-184 as a white solid. ESI-MS found 1160.3, C₂₁₅H₃₂₇N₄₇O₆₅S (M+4H⁺) requires 1159.8.

### Example 185: Compound 78

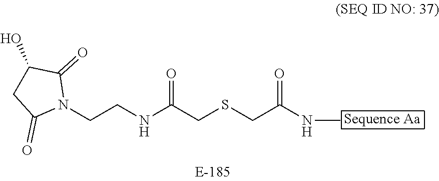 [SMILES: CNC(=O)CSCC(=O)NCCN1C(=O)C[C@H](O)C1=O]

E-185 was prepared according to GP4 using 34 mg of resin (estimated loading 0.125 mmol/g) and I-398 to afford 2.5 mg of E-185 as a white solid. ESI-MS found 1157.2, C₂₁₃H₃₂₃N₄₇O₆₆S (M+4H⁺) requires 1156.8.

### Example 186: Compound 79

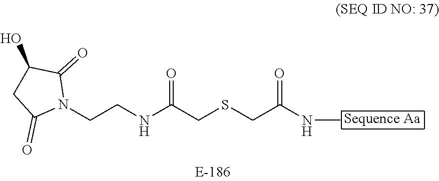 [SMILES: CNC(=O)CSCC(=O)NCCN1C(=O)C[C@@H](O)C1=O]

E-186 was prepared according to GP4 using 35.3 mg of resin (estimated loading 0.125 mmol/g) and I-399 to afford 1.3 mg of E-186 as a white solid. ESI-MS found 1157.3, C₂₁₃H₃₂₃N₄₇O₆₆S (M+4H⁺) requires 1156.8.

### Example 187: Compound 63

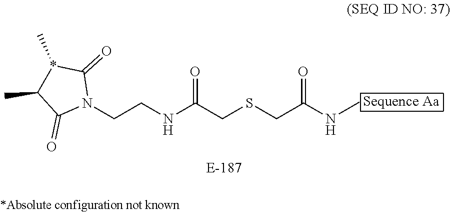

E-187 was prepared according to GP4 using 39 mg of resin (estimated loading 0.125 mmol/g) and I-404 to afford 3.0 mg of E-187 as a white solid. ESI-MS found 1160.3, C₂₁₅H₃₂₇N₄₇O₆₅S (M+4H⁺) requires 1159.8.

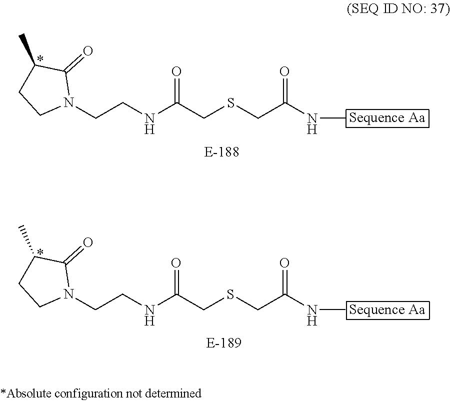 [SMILES: CNC(=O)CSCC(=O)NCCN1CC[C@@H](C)C1=O ; CNC(=O)CSCC(=O)NCCN1CC[C@H](C)C1=O]

### Example 188: Compound 69

E-188 was prepared according to GP4 using 45.9 mg of resin (estimated loading 0.125 mmol/g) and (R*)-I-409 to afford 4 mg of E-188 as a white solid. ESI-MS found 1153.3, C₂₁₄H₃₂₇N₄₇O₆₄S (M+4H⁺) requires 1152.8.

### Example 189: Compound 68

E-189 was prepared according to GP4 using 43.8 mg of resin (estimated loading 0.125 mmol/g) and (S*)-I-409 to afford 4.3 mg of E-189 as a white solid. ESI-MS found 1153.3, C₂₁₄H₃₂₇N₄₇O₆₄S (M+4H⁺) requires 1152.8.

### Example 190: Compound 77

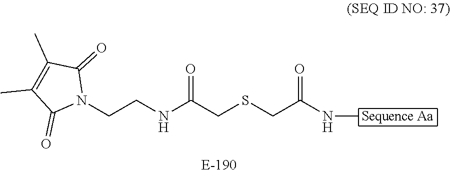

E-190 was prepared according to GP4 using 36.6 mg of resin (estimated loading 0.125 mmol/g) and I-412 to afford 0.5 mg of E-190 as a white solid. ESI-MS found 1159.7, C₂₁₅H₃₂₅N₄₇O₆₅S (M+4H⁺) requires 1159.3.

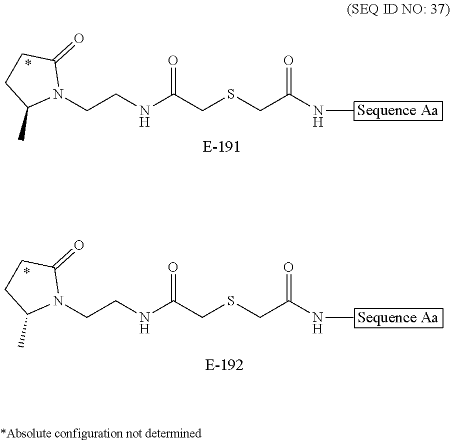 [SMILES: CNC(=O)CSCC(=O)NCCN1C(=O)CC[C@@H]1C ; CNC(=O)CSCC(=O)NCCN1C(=O)CC[C@H]1C]

### Example 191: Compound 75

E-191 was prepared according to GP4 using 30.2 mg of resin (estimated loading 0.125 mmol/g) and (S*)-I-413 to afford 2.9 mg of E-191 as a white solid. ESI-MS found 1153.4, C₂₁₄H₃₂₇N₄₇O₆₄S (M+4H⁺) requires 1152.8.

### Example 192: Compound 74

E-192 was prepared according to GP4 using 31.1 mg of resin (estimated loading 0.125 mmol/g) and (R*)-I-413 to afford 3.3 mg of E-192 as a white solid. ESI-MS found 1153.4, C₂₁₄H₃₂₇N₄₇O₆₄S (M+4H⁺) requires 1152.8.

### Example 193: Compound 67

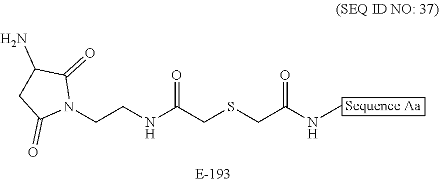 [SMILES: CNC(=O)CSCC(=O)NCCN1C(=O)CC(N)C1=O]

E-193 was prepared according to GP4 using 41.4 mg of resin (estimated loading 0.125 mmol/g) and I-417 to afford 4.1 mg of E-193 as a white solid. ESI-MS found 1157.1, C₂₁₃H₃₂₄N₄₈O₆₅S (M+4H⁺) requires 1156.6.

### Example 194: Compound 42

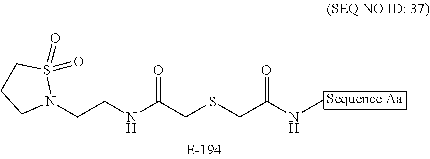 [SMILES: CNC(=O)CSCC(=O)NCCN1CCCS1(=O)=O]

E-194 was prepared according to GP4 using 42.1 mg of resin (estimated loading 0.125 mmol/g) and I-421 to afford 5.4 mg of E-194 as a white solid. ESI-MS found 1158.8, C₂₁₂H₃₂₅N₄₇O₆₅S₂ (M+4H⁺) requires 1158.3.

### Example 195: Compound 40

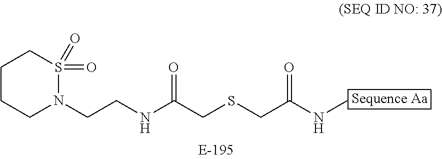 [SMILES: CNC(=O)CSCC(=O)NCCN1CCCCS1(=O)=O]

E-195 was prepared according to GP4 using 50.9 mg of resin (estimated loading 0.125 mmol/g) and I-425 to afford 5.8 mg of E-195 as a white solid. ESI-MS found 1162.3, C₂₁₃H₃₂₇N₄₇O₆₅S₂ (M+4H⁺) requires 1161.8.

### Example 196: Compound 153

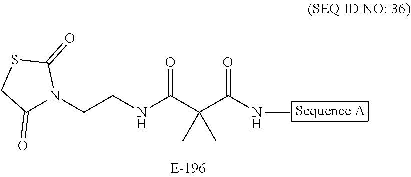

Peptide E-196 was prepared from 24.9 mg of resin (approximate loading 0.18 mmol/g) using I-426 and GP4 to afford 2.7 mg of E-196 as a white solid. ESI-MS found 1059.4989, C₁₉₂H₂₇₈SN₄₆O₆₁ (M−4H)⁻ requires 1059.0.

### C2. Synthesis of Additional Intermediates to Compounds Described in this Invention

The synthesis of carboxylic acid I-430 is depicted in Scheme 138:

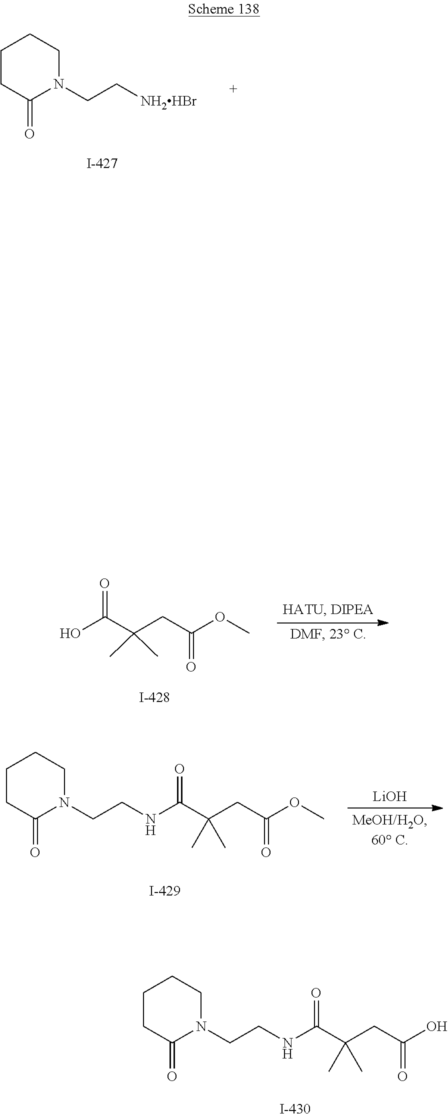

To a solution of amine salt I-427 (165.8 mg, 0.743 mmol), carboxylic acid I-428 (119 mg 1.0 equiv.) and DIPEA (388 μL, 3.0 equiv.) in DMF (2 mL) at ambient temperature was added HATU (423.8 mg, 1.5 equiv.). The reaction mixture was maintained at ambient temperature for 23 hours and was then purified by preparative HPLC (Mobile Phase: MeCN/H₂O with 0.1% TFA) to afford ester I-429 as a colorless oil (180.7 mg, 86% yield).

To a solution of ester I-429 (90.3 mg, 0.318 mmol) in MeOH (3 mL) and H₂O (1 mL) was added lithium hydroxide (15.2 mg, 2.0 equiv.). The reaction mixture was heated to 60° C. for 23 hours, acidified with 6 N HCl to pH<2, and then purified by preparative HPLC (Mobile Phase: MeCN/H₂O with 0.1% TFA) to afford acid I-430 as a white solid (26.1 mg 30% yield). ESI-MS found 271.3, C₁₃H₂₃N₂O₄ (M+4H)⁴⁺ requires 271.3.

The syntheses of compounds I-439 & I-443 involved 8 steps as depicted in the following Scheme 139.

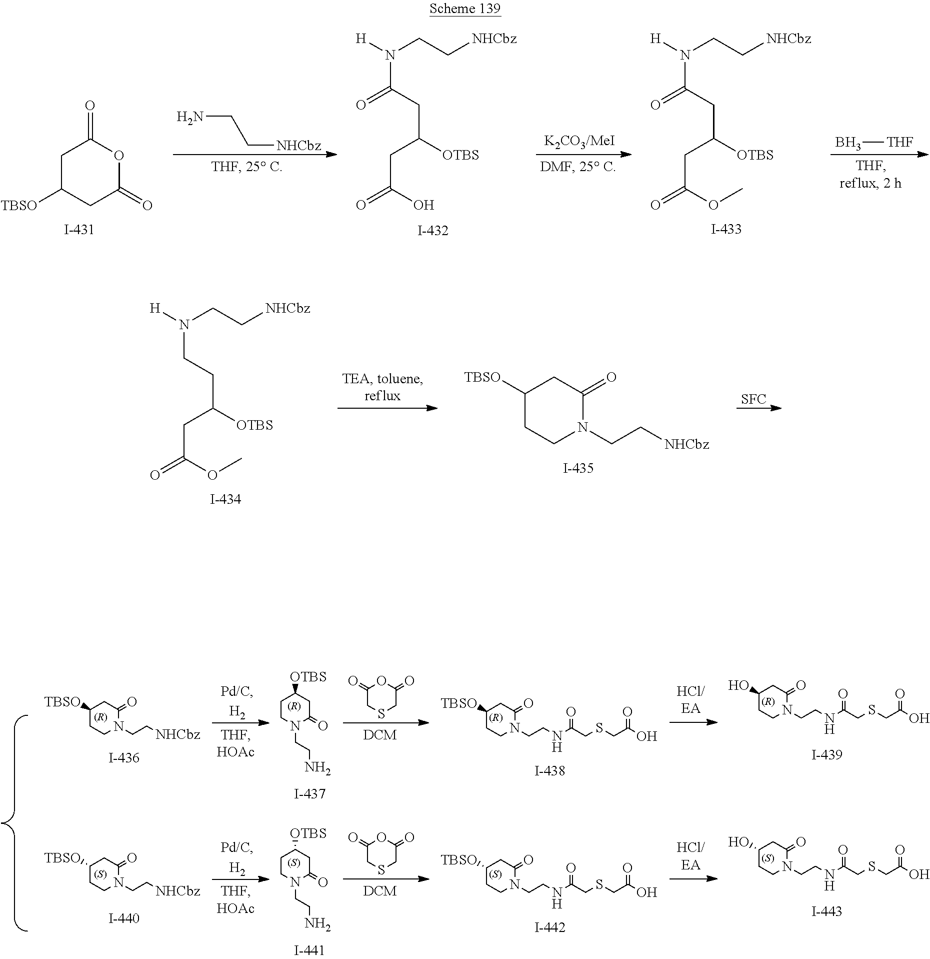

### Step 1. 5-((2-(((benzyloxy)carbonyl)amino)ethyl)amino)-3-((tert-butyldimethylsilyl)oxy)-5-oxopentanoic acid (I-432)

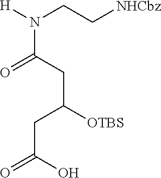 [SMILES: CC(C)(C)[Si](C)(C)OC(CC(=O)O)CC(=O)NCCNC(=O)OCc1ccccc1]

A solution of 4-((tert-butyldimethylsilyl)oxy)dihydro-2H-pyran-2,6(3H)-dione I-431 (cas: 91424-40-7, 5.0 g, 20.5 mmol), benzyl(2-aminoethyl)carbamate (cas: 72080-83-2, 3.97 g 20.5 mmol) and TEA (6.2 g, 61.5 mmol) in anhydrous THF (150 mL) was stirred at 25° C. for 16 hours. The reaction mixture was concentrated to afford the crude 5-((2-(((benzyloxy)carbonyl)amino)ethyl)amino)-3-((tert-butyldimethylsilyl)oxy)-5-oxopentanoic acid I-432 (8.9 g), which was taken on to the next reaction without further purification. MS (ESI, positive ion) m/z: 439.2 (M+1).

### Step 2. methyl 5-((2-(((benzyloxy)carbonyl)amino)ethyl)amino)-3-((tert-butyldimethylsilyl)oxy)-5-oxopentanoate (I-433)

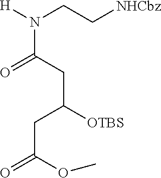 [SMILES: COC(=O)CC(CC(=O)NCCNC(=O)OCc1ccccc1)O[Si](C)(C)C(C)(C)C]

To a mixture of acid I-432 (5 g, 11.4 mmol) and K₂CO₃ (2.4 g, 17.1 mmol) in anhydrous DMF (30 mL) was added MeI (1.95 g, 13.7 mmol) at 25° C. under N₂. The reaction mixture was heated to 50° C. for 5 hours, then concentrated and dissolved in EtOAc (150 mL). The organic phase was washed with water (50 mL×3), dried and concentrated to afford the crude product, which was purified by silica gel chromatography (Petroleum ether:EtOAc=3:1 to 1:1) to provide a pale oil (4.5 g, 87% yield). MS (ESI, positive ion) m/z: 453.2 (M+1).

### Step 3. methyl 5-((2-(((benzyloxy)carbonyl)amino)ethyl)amino)-3-((tert-butyldimethylsilyl)oxy) pentanoate (I-434)

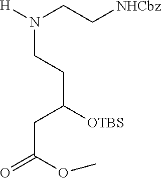 [SMILES: COC(=O)CC(CCNCCNC(=O)OCc1ccccc1)O[Si](C)(C)C(C)(C)C]

To a solution of ester I-433 (2 g, 4.4 mmol) in anhydrous THF (200 mL) was added BH₃-THF (1 M, 22 mL, 22 mmol) at 0° C. The reaction mixture was heated to 80° C. for 2 hours and cooled to 0° C., then quenched by adding 1 mL of concentrated HCl. The reaction mixture was concentrated and dissolved in EtOAc (150 mL). The organic phase was washed with water (50 mL×3), dried and concentrated to afford the crude amine I-434 (2.5 g), which was taken to the next reaction without further purification. MS (ESI, positive ion) m/z: 439.2 (M+1).

### Step 4. Benzyl(2-(4-((tert-butyldimethylsilyl)oxy)-2-oxopiperidin-1-yl)ethyl)carbamate (I-435)

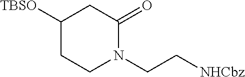 [SMILES: CC(C)(C)[Si](C)(C)OC1CCN(CCNC(=O)OCc2ccccc2)C(=O)C1]

A solution of amine I-434 (2.5 g, 5.7 mmol) and TEA (2.89 g, 28.5 mmol) in anhydrous toluene (150 ml) was heated to 50° C. for 16 hours. The reaction mixture was concentrated and purified by preparative HPLC (Mobile Phase: MeCN/H₂O with 0.1% TFA) to afford a brown oil (430 mg, 19% yield). MS (ESI, positive ion) m/z: 407.2 (M+1).

Step 5. Chiral SFC separation [5.0 cm I.D.×25 cm L CHIRALCEL OZ column at 35° C., flow rate 60 mL/min with Hexane/IPA=80/20 (V/V)] afforded enantiomers I-436 and I-440

### Step 6. (R or S)-1-(2-aminoethyl)-4-((tert-butyldimethylsilyl)oxy) piperidin-2-one (peak1)(I-437 or I-441)

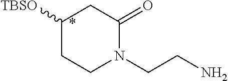

To a solution of benzyl (R or S)-(2-(4-((tert-butyldimethylsilyl)oxy)-2-oxopiperidin-1-yl)ethyl)carbamate I-436 (150 mg, 6.82 mmol) in THF (10 ml) was added Pd/C (170 mg) quickly under H₂. The reaction was then stirred at ambient temperature for 16 hours, when analysis by LC-MS indicated reaction completion. The reaction mixture was filtered, and the filtrate was concentrated under reduced pressure to afford crude amine I-437 (105 mg), which was taken to the next reaction without further purification. MS (ESI, positive ion) m/z: 273.2 (M+1).

### Step 7. (R or S)-2-(2-(3-methoxy-2-oxopyrrolidin-1-yl)ethyl)isoindoline-1,3-dione (I-438 or I-442)

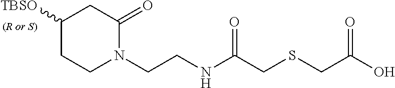 [SMILES: CC(C)(C)[Si](C)(C)OC1CCN(CCNC(=O)CSCC(=O)O)C(=O)C1]

A solution of (R or S)-1-(2-hydroxyethyl)-3-methoxypyrrolidin-2-one (I-437) (100 mg 0.94 mmol) and thiodiglycolic anhydride (cas: 3261-87-8, 240 mg, 1.84 mmol) in anhydrous THF (5 mL) was stirred at 25° C. for 16 hours. The reaction mixture was concentrated to afford crude acid I-438 (340 mg), which was taken to the next reaction without further purification. MS (ESI, positive ion) m/z: 405.1 (M+1).

### Step 8. (R or S)-2-((2-((2-(4-hydroxy-2-oxopiperidin-1-yl)ethyl)amino)-2-oxoethyl)thio)acetic acid (I-439)

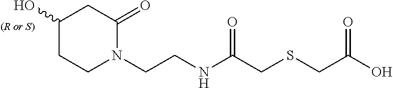 [SMILES: O=C(O)CSCC(=O)NCCN1CCC(O)CC1=O]

To a solution of crude acid I-438 (340 mg) in THF (10 mL) was added HCl/EtOAc (5 M, 2 mL) at 0° C. The reaction mixture was stirred for 12 hours at ambient temperature, then concentrated to afford a residue, which was purified by preparative HPLC (Mobile Phase: MeCN/H₂O with 0.1% TFA) to afford acid I-439 as a pale oil (22 mg, 9% yield). MS (ESI, positive ion) m/z: 291.2 (M+1).). ¹H NMR (400 MHz, D₂O) δ 4.05-4.03 (m, 1H), 3.44-3.24 (m, 6H), 3.22 (s, 2H), 3.17 (s, 2H), 2.55-2.50 (m, 1H), 2.20-2.15 (m, 1H), 1.91-1.87 (m, 1H), 1.73-1.68 (m, 1H).

A similar sequence of steps with carbamate I-441 afforded acid I-443 (40 mg, 16% yield) as a pale oil.

The syntheses of intermediates I-451 and I-454 involved 8 steps as depicted in the following Scheme 140.

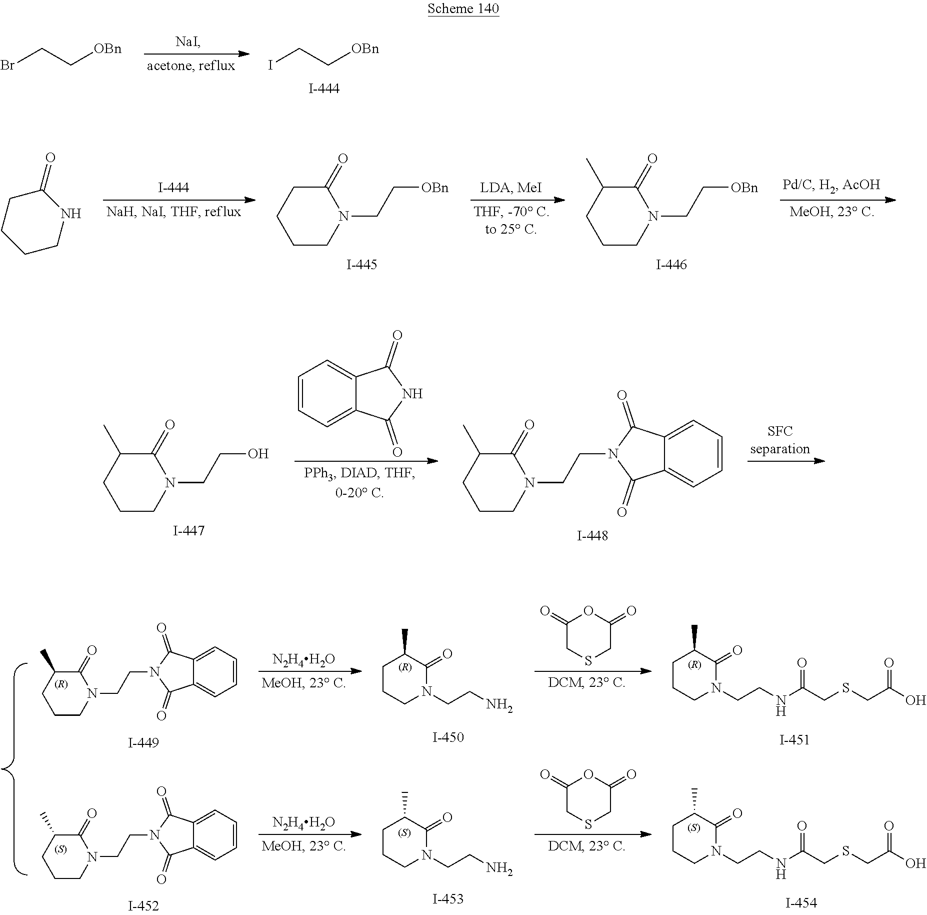

### Step 1. ((2-iodoethoxy)methyl)benzene (I-444)

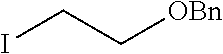 [SMILES: ICCOCc1ccccc1]

A mixture of ((2-bromoethoxy)methyl)benzene (cas: 1462-37-9, 10 g, 46.7 mmol) and sodium iodide (10.5 g, 1.5 equiv.) in acetone (250 mL) was heated to reflux for 16 hours. The reaction mixture was concentrated, and the residue was dissolved into EtOAc (200 mL) and diluted with water (100 mL). The organic layer was washed with brine (50 mL×3), dried over anhydrous Na₂SO₄ and concentrated to afford the crude product, which was purified by silica gel chromatography (Petroleum ether:EtOAc=20:1) to afford iodide I-444 as a brown liquid (7 g, yield: 60%).

### Step 2. 1-(2-(benzyloxy)ethyl)piperidin-2-one (I-445)

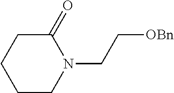 [SMILES: O=C1CCCCN1CCOCc1ccccc1]

A solution of 2-piperidone (cas: 675-20-7, 2 g, 20.2 mmol) in anhydrous THF (50 mL) was added NaH (60% in mineral oil, 1.2 g, 30.3 mmol) at 0° C., then iodide I-444 (10.6 g 40.4 mmol). The reaction mixture was heated at 80° C. for 16 hours, then quenched by adding 100 mL of saturated aqueous NH₄Cl at 0° C. The aqueous phase was extracted with EtOAc (50 mL×3). The organic extracts were combined, dried and concentrated afford the crude product, which was purified by silica gel chromatography (DCM:MeOH=20:1) to afford amide I-445 (1.2 g, yield: 25%) as a pale oil. MS (ESI, positive ion) m/z: 234.2 (M+1).

### Step 3. 1-(2-(benzyloxy)ethyl)-3-methylpiperidin-2-one (I-446)

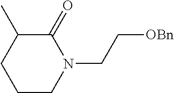 [SMILES: CC1CCCN(CCOCc2ccccc2)C1=O]

To a solution of amide I-445 (500 mg, 2.14 mmol) in anhydrous THF (20 mL) was added LDA (2 M, 1.6 mL, 3.2 mmol) at −78° C. under N₂. The reaction mixture was stirred for 30 minutes at the same temperature and MeI (914 mg, 6.44 mmol) was added. After 1 hour, LC-MS analysis indicated reaction completion. The reaction mixture was quenched with H₂O (20 mL) and extracted with EtOAc (20 mL×3). The combined organic extracts was concentrated to obtain crude product, which was purified by silica gel chromatography (DCM:MeOH=20:1) to provide lactam I-446 as a brown oil (380 mg, 72% yield). MS (ESI, positive ion) m/z: 248.2 (M+1).

### Step 4. 1-(2-hydroxyethyl)-3-methylpiperidin-2-one (I-447)

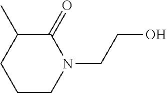

To a solution of lactam I-446 (5 g, 20.24 mmol) and acetic acid (0.5 mL) in MeOH (150 mL) was added 10% Pd(OH)₂ (300 mg) under H₂. The reaction mixture was heated to 50° C. under H₂ (3 atm) for 2 days. The reaction mixture was filtered, and the filtrate was concentrated to afford the crude product (3.2 g), which was taken to the next reaction without further purification. MS (ESI, positive ion) m/z: 158.2 (M+1).

### Step 5. 2-(2-(3-methyl-2-oxopiperidin-1-yl)ethyl)isoindoline-1,3-dione (I-448)

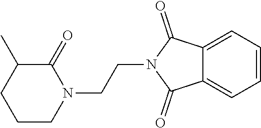 [SMILES: CC1CCCN(CCN2C(=O)c3ccccc3C2=O)C1=O]

A solution of lactam I-447 (200 mg, 1.27 mmol), phthalimide (374 mg, 2.54 mmol) and P(n-Bu)₃ (514 mg, 2.54 mmol) in anhydrous THF (10 mL) was stirred at 0° C. for 0.5 hours. Then DIAD (514 mg, 2.54 mmol) was added to the reaction dropwise and the reaction mixture was stirred for 18 hours at ambient temperature. The reaction mixture was concentrated to get crude product, which was purified by silica gel chromatography (Petroleum ether:EtOAc=1:1) to provide imide I-448 as a pale oil (180 mg, 49% yield). MS (ESI, positive ion) m/z: 287.2 (M+1).

Step 6. Phthalimide I-448 was subjected to chiral SFC to separate the racemate into its constituent enantiomers. Separation was accomplished using a Superchiral S-AD (Chiralway) column (2.1 cm I.D.×25 cm L, 5 μm at 35° C., flow rate 10 mL/min with Hexane/EtOH=55/45 (v/v), 30 mg injection) affording 137 mg of each enantiomer (I-449 and I-452) from 400 mg of racemate.

### Step 7. (R or S)-2-(2-(3-methoxy-2-oxopyrrolidin-1-yl)ethyl)isoindoline-1,3-dione (I-450)

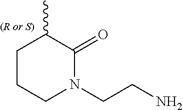

To a solution of I-449 (150 mg, 0.52 mmol) in MeOH (10 mL) was added Hydrazine hydrate (cas: 7803-57-8, 80%, 0.3 mL) at 0° C. The reaction mixture was stirred for 12 hours at ambient temperature, then filtered and the filtrate was concentrated. The residue afforded (210 mg) was used directly in the next step. MS (ESI, positive ion) m/z: 157.2 (M+1).

### Step 8. (R or S)-2-((2-((2-(3-methyl-2-oxopiperidin-1-yl)ethyl)amino)-2-oxoethyl)thio)acetic acid (I-451)

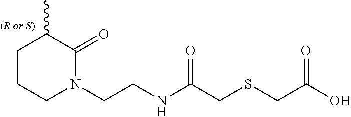 [SMILES: CC1CCCN(CCNC(=O)CSCC(=O)O)C1=O]

A solution of (R or S)-1-(2-aminoethyl)-3-methylpiperidin-2-one, I-450 (210 mg, 1.36 mmol) and thiodiglycolic anhydride (533 mg, 4.08 mmol) in 5 mL of DCM was stirred at 25° C. for 16 hours. The reaction mixture was concentrated to afford the crude product, which was purified by preparative HPLC (Mobile Phase: MeCN/H₂O (with 0.1% TFA)) to afford acid I-451 (40 mg, 11% yield) as a pale oil. MS (ESI, positive ion) m/z: 289.2 (M+1). ¹H NMR (400 MHz, D2O) δ 3.43-3.39 (m, 2H), 3.36-3.28 (m, 6H), 3.24 (s, 2H), 2.36-2.31 (m, 1H), 1.80-1.70 (m, 2H), 1.66-1.58 (m, 1H), 1.46-1.39 (m, 1H), 1.08-1.06 (d, 3H).

A similar sequence of steps from phthalimide I-452 afforded acid I-454 (43 mg, 12% yield) as a pale oil.

The synthesis of intermediate I-458 is depicted in Scheme 141:

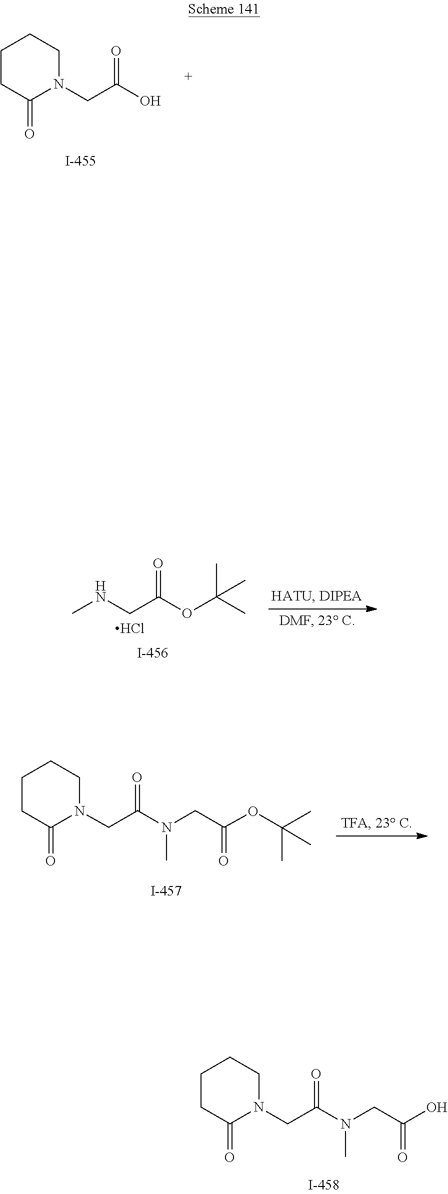

To a solution of amine salt I-456 (57.3 mg, 0.315 mmol), carboxylic acid I-455 (49.5 mg 1.0 equiv.) and DIPEA (165 μL, 3.0 equiv.) in DMF (1 mL) at ambient temperature was added HATU (179.7 mg, 1.5 equiv.). The reaction mixture was maintained at ambient temperature for 2 hours and was then purified by preparative HPLC (Mobile Phase: MeCN/H₂O with 0.1% AcOH) to afford ester I-457 as a colorless oil (75.3 mg, 84% yield).

To ester I-457 (75.3 mg, 0.265 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-458 as a white solid (42.3 mg, 70% yield). ESI-MS found 227.1. C₁₀H₁₅N₂O₄ (M−H)⁻ requires 227.1.

The synthesis of intermediate I-461 is depicted in Scheme 142:

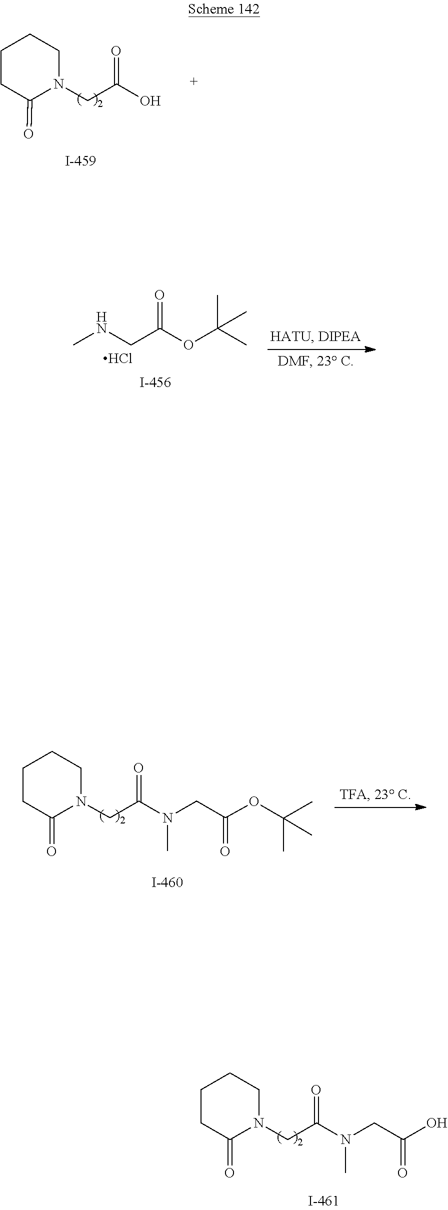

To a solution of amine salt I-456 (57.1 mg, 0.314 mmol), carboxylic acid I-455 (53.8 mg 1.0 equiv.) and DIPEA (164 μL, 3.0 equiv.) in DMF (1 mL) at ambient temperature was added HATU (179.3 mg, 1.5 equiv.). The reaction mixture was maintained at ambient temperature for 3 hours and was then purified by preparative HPLC (Mobile Phase: MeCN/H₂O with 0.1% AcOH) to afford ester I-460 as a colorless oil (81.1 mg, 87% yield).

To ester I-460 (81.1 mg, 0.272 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-461 as a white solid (73.8 mg, quantitative yield). ESI-MS found 243.3. C₁₁H₁₉N₂O₄ (M+H)⁺ requires 243.1.

### The Synthesis of Intermediate I-464 is Depicted in Scheme 143

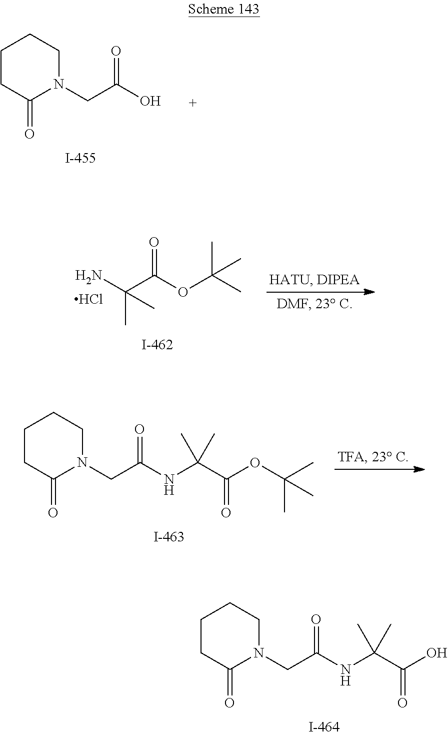 [SMILES: O=C(O)CN1CCCCC1=O ; Cl ; CC(C)(C)OC(=O)C(C)(C)N ; CC(C)(C)OC(=O)C(C)(C)NC(=O)CN1CCCCC1=O ; CC(C)(NC(=O)CN1CCCCC1=O)C(=O)O]

To a solution of amine salt I-462 (96.6 mg, 0.493 mmol), carboxylic acid I-455 (77.5 mg 1.0 equiv.) and DIPEA (258 μL, 3.0 equiv.) in DMF (1 mL) at ambient temperature was added HATU (281.4 mg, 1.5 equiv.). The reaction mixture was maintained at ambient temperature for 16 hours and was then purified by preparative HPLC (Mobile Phase: MeCN/H₂O with 0.1% AcOH) to afford ester I-463 as a white solid (127.6 mg, 87% yield).

To ester I-463 (127.6 mg, 0.428 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-464 as a white solid (115.7 mg, quantitative yield). ESI-MS found 243.3. C₁₁H₁₉N₂O₄ (M+H)⁺ requires 243.1.

### The Synthesis of Intermediate I-466 is Depicted in Scheme 144

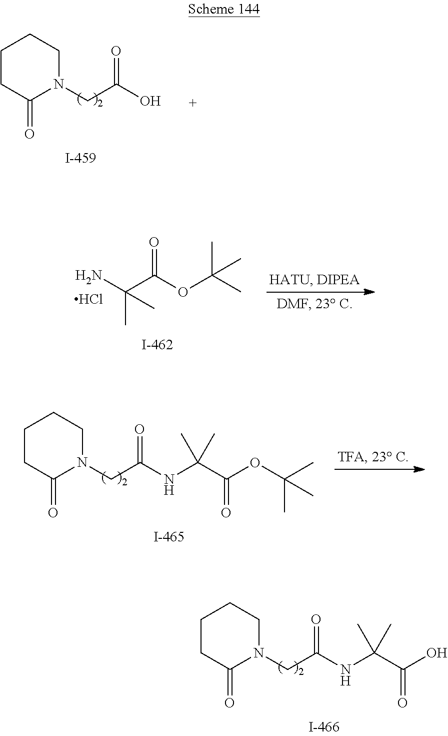

To a solution of amine salt I-462 (93.1 mg, 0.476 mmol), carboxylic acid I-459 (81.4 mg 1.0 equiv.) and DIPEA (249 μL, 3.0 equiv.) in DMF (2 mL) at ambient temperature was added HATU (271.2 mg, 1.5 equiv.). The reaction mixture was maintained at ambient temperature for 3 hours and was then purified by preparative HPLC (Mobile Phase: MeCN/H₂O with 0.1% AcOH) to afford ester I-465 as a colorless oil (133 mg, 90% yield).

To ester I-465 (133 mg, 0.426 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-466 as a white solid (108.4 mg, >99% yield). ESI-MS found 257.3. C₁₂H₂₁N₂O₄ (M+H)⁺ requires 257.1.

The synthesis of intermediate I-469 is depicted in Scheme 145:

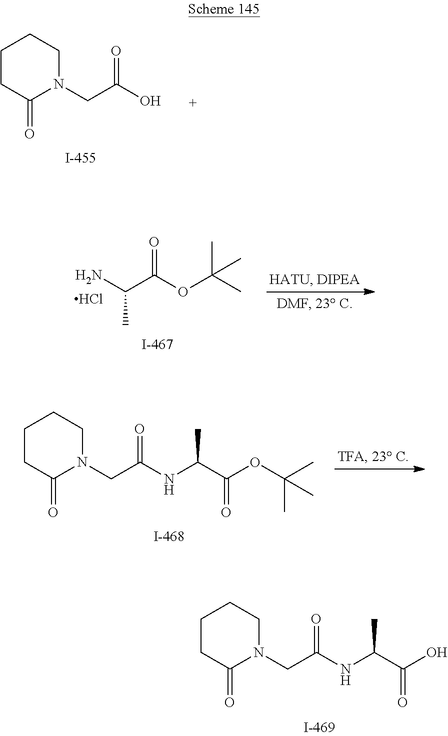

To a solution of amine salt I-467 (80.1 mg, 0.441 mmol), carboxylic acid I-455 (69.3 mg 1.0 equiv.) and DIPEA (231 μL, 3.0 equiv.) in DMF (2 mL) at ambient temperature was added HATU (251.6 mg, 1.5 equiv.). The reaction mixture was maintained at ambient temperature for 16 hours and was then purified by preparative HPLC (Mobile Phase: MeCN/H₂O with 0.1% AcOH) to afford ester I-468 as a colorless oil (112.4 mg, 90% yield).

To ester I-468 (112.4 mg, 0.395 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-469 as a white solid (79.1 mg, 88% yield). ESI-MS found 229.2. C₁₀H₁₇N₂O₄ (M+H)⁺ requires 229.1.

The synthesis of intermediate I-471 is depicted in Scheme 146:

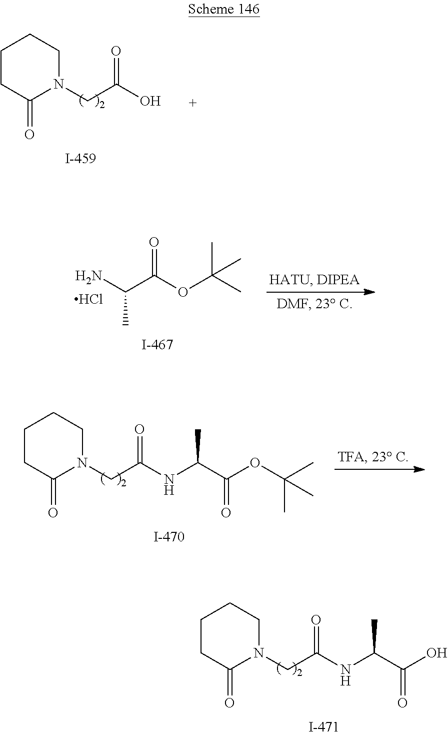

To a solution of amine salt I-467 (87.9 mg, 0.484 mmol), carboxylic acid I-459 (82.8 mg 1.0 equiv.) and DIPEA (253 μL, 3.0 equiv.) in DMF (2 mL) at ambient temperature was added HATU (275.8 mg, 1.5 equiv.). The reaction mixture was maintained at ambient temperature for 16 hours and was then purified by preparative HPLC (Mobile Phase: MeCN/H₂O with 0.1% AcOH) to afford ester I-470 as a colorless oil (131.9 mg, 91% yield).

To ester I-470 (131.9 mg, 0.442 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-471 as a white solid (102.2 mg, 95% yield). ESI-MS found 243.3. C₁₁H₁₉N₂O₄ (M+H)⁺ requires 243.1.

The synthesis of intermediate I-474 is depicted in Scheme 147:

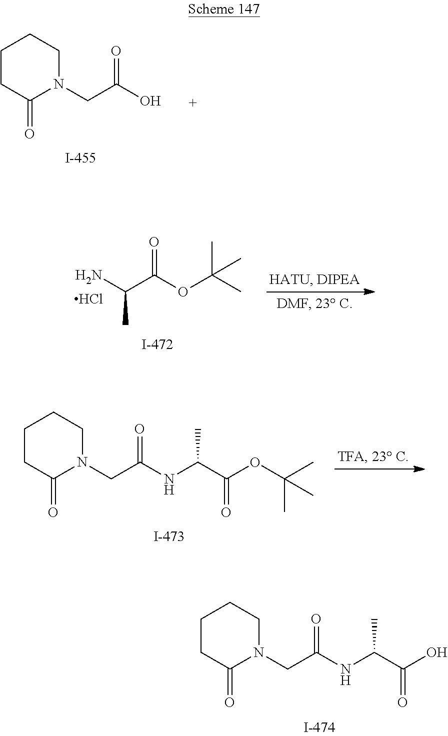

To a solution of amine salt I-472 (79.1 mg, 0.436 mmol), carboxylic acid I-455 (68.4 mg 1.0 equiv.) and DIPEA (228 μL, 3.0 equiv.) in DMF (2 mL) at ambient temperature was added HATU (248.4 mg, 1.5 equiv.). The reaction mixture was maintained at ambient temperature for 16 hours and was then purified by preparative HPLC (Mobile Phase: MeCN/H₂O with 0.1% AcOH) to afford ester I-473 as a colorless oil (105.9 mg, 86% yield).

To ester I-473 (105.9 mg, 0.372 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-474 as a white solid (100.5 mg, quantitative yield). ESI-MS found 229.2. C₁₀H₁₇N₂O₄ (M+H)⁺ requires 229.1.

The synthesis of intermediate I-476 is depicted in Scheme 148:

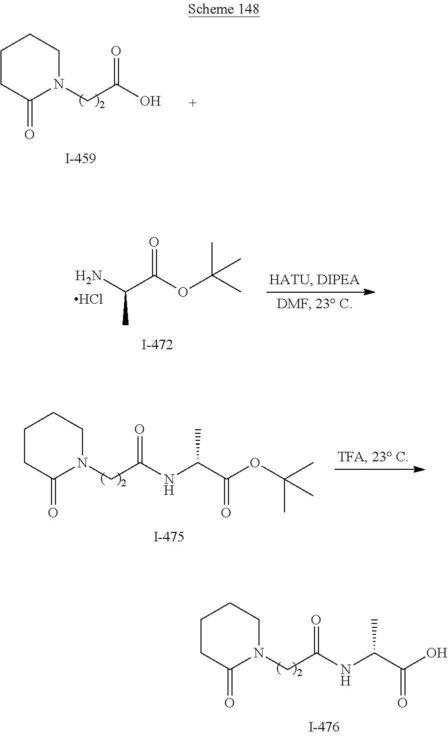

To a solution of amine salt I-472 (74.4 mg, 0.41 mmol), carboxylic acid I-459 (70.1 mg 1.0 equiv.) and DIPEA (214 μL, 3.0 equiv.) in DMF (2 mL) at ambient temperature was added HATU (233.6 mg, 1.5 equiv.). The reaction mixture was maintained at ambient temperature for 16 hours and was then purified by preparative HPLC (Mobile Phase: MeCN/H₂O with 0.1% AcOH) to afford ester I-475 as a colorless oil (104.3 mg, 85% yield).

To ester I-475 (104.3 mg, 0.35 mmol) was added TFA (4 mL). The reaction mixture was maintained at ambient temperature for 4 hours, then concentrated. The residue afforded was purified by preparative HPLC (Mobile Phase: MeCN/H₂O with 0.1% TFA) to afford acid I-476 as a white solid (96.6 mg, quantitative yield). ESI-MS found 243.2. C₁₁H₁₉N₂O₄ (M+H)⁺ requires 243.1.

### The Synthesis of Intermediate I-478 is Depicted in Scheme 149

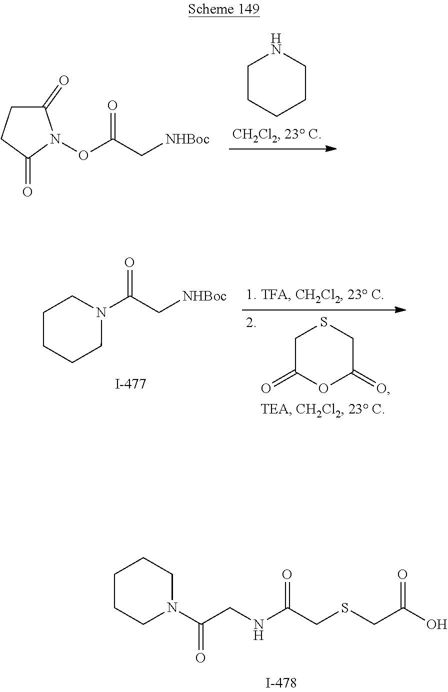 [SMILES: CC(C)(C)OC(=O)NCC(=O)ON1C(=O)CCC1=O ; C1CCNCC1 ; CC(C)(C)OC(=O)NCC(=O)N1CCCCC1 ; O=C1CSCC(=O)O1 ; O=C(O)CSCC(=O)NCC(=O)N1CCCCC1]

To a solution of 2,5-dioxopyrrolidin-1-yl (tert-butoxycarbonyl)glycinate (50 mg, 0.183 mmol) in 1 mL of anhydrous DCM was added piperidine (0.2 mL, 5 equiv.). The reaction mixture was stirred at room temperature for 18 hours, then diluted with 5 mL of DCM, washed with 1N HCl (5 mL), saturated aqueous NaHCO₃ (5 mL) and brine (5 mL). The organic extracts were dried (Na₂SO₄) and concentrated to afford a residue, which was purified by silica gel chromatography to afford the amide I-477 (35 mg, 81% yield).

Amide I-477 (35 mg, 0.15 mmol) was dissolved in 1:1 DCM/TFA (3 mL). After 1 hour at room temperature, the reaction mixture was concentrated and held under high vacuum for 1 hour. The resulting oil and thiodiglycolic anhydride (21.5 mg, 1.1 equiv.) were dissolved in DCM (3 mL), and TEA (65 μL, 2.5 equiv.) was added. After 25 minutes, the reaction mixture was concentrated and purified by preparative HPLC (H₂O/MeCN with 0.1% TFA) to afford 14.6 mg (5% yield) of acid I-478. ESI-MS found 275.1, C₁₁H₁₉N₂O₄S (MH⁺) requires 275.1.

The synthesis of intermediate I-486 is depicted in Scheme 150:

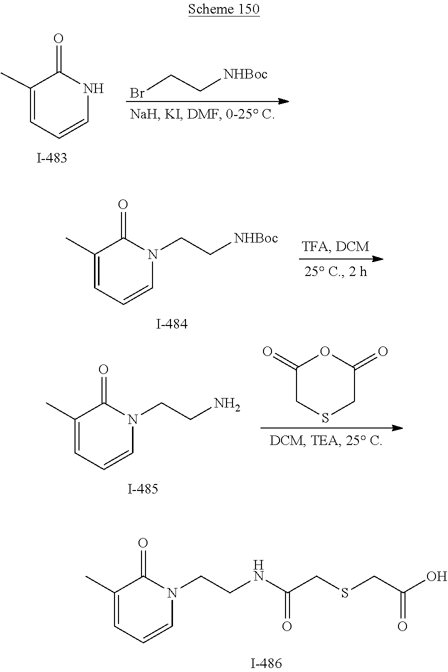

### Step 1: Tert-butyl (2-(3-methyl-2-oxopyridin-1(2H)-yl)ethyl)carbamate (I-484)

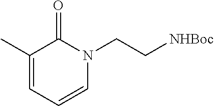 [SMILES: Cc1cccn(CCNC(=O)OC(C)(C)C)c1=O]

A solution of 3-methylpyridin-2(1H)-one, I-483 (cas: 1003-56-1, 3.0 g, 27.52 mmol, 1.0 equiv.), KI (0.913 g, 5.50 mmol, 0.2 equiv.), NaH (60%, 1.321 g, 33.02 mmol, 1.2 equiv.), in DMF (20 mL) was stirred under nitrogen at 0° C. for 1 hour. Then the reaction mixture was added to a solution of tert-butyl (2-bromoethyl)carbamate (cas: 39684-80-5, 9.205 g 41.28 mmol, 1.5 equiv.) in DMF (20 mL) dropwise. The reaction mixture was stirred at 0° C. for other 1 hour and then 25° C. for another 48 hours. LCMS analysis showed ˜50% conversion. Then H₂O (50 mL) was added and the mixture was extracted with EtOAc (100 mL×3). The organic phase was dried and purified by silica gel chromatography (20%-50% EtOAc with Petroleum ether) to afford the product tert-butyl (2-(3-methyl-2-oxopyridin-1(2H)-yl)ethyl)carbamate I-484 (1.2 g, 17% yield, 95% purity) as white solid, MS (ESI, positive ion) m/z: 253 (M+1) and byproduct: tert-butyl (2-((3-methylpyridin-2-yl)oxy)ethyl)carbamate (0.2 g, 3% yield, 95% purity), MS (ESI, positive ion) m/z: 253.1 (M+1).

### Step 2:1-(2-aminoethyl)-3-methylpyridin-2(1H)-one (I-485)

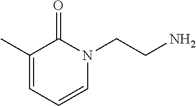

To the solution of tert-butyl (2-(3-methyl-2-oxopyridin-1(2H)-yl)ethyl)carbamate I-484 (1.2 g, 4.76 mmol, 1.0 equiv.) in DCM (20 mL) was added TFA (10 mL). The solution was stirred for 2 h at 25° C. After completion, the mixture was concentrated and the remained TFA was removed by centrifugation to give light yellow oil (0.7 g, TFA salt, 97% yield, 60% purity by LCMS). MS (ESI, positive ion) m/z: 153.1 (M+1).

### Step 3: 2-((2-((2-(3-methyl-2-oxopyridin-1(2H)-yl)ethyl)amino)-2-oxoethyl)thio)acetic acid (I-486)

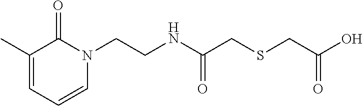 [SMILES: Cc1cccn(CCNC(=O)CSCC(=O)O)c1=O]

A solution of 1-(2-aminoethyl)-3-methylpyridin-2(1H)-one (0.7 g, 4.61 mmol, 1.0 equiv.), TEA (1.397 g, 13.83 mmol, 3.0 equiv.), thiodiglycolic anhydride (0.913 g, 6.92 mmol, 1.5 equiv.) in DCM (10 mL) was stirred for 36 hours at 25° C. After completion, the reaction mixture was concentrated. The residue was purified by Preparative HPLC (Mobile Phase: ACN-H₂O) to afford I-486 (119.7 mg, 9% yield, 99.2% purity) as a white solid. MS (ESI, positive ion) m/z: 285.1 (M+1). ¹H NMR (400 MHz, D₂O) δ 7.41-7.36 (m, 1H), 7.32 (dd, 1H), 6.31 (t, 1H), 4.07-4.01 (m, 2H), 3.54-3.48 (m, 2H), 3.17 (d, 4H), 1.99 (s, 3H).

### The Synthesis of Intermediate I-490 is Depicted in Scheme 151

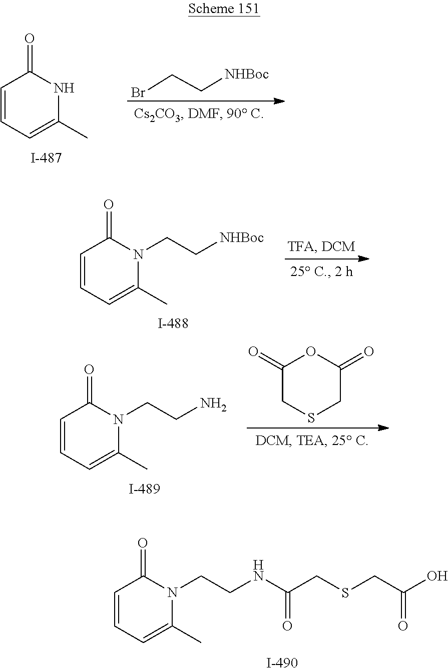

### Step 1: tert-butyl (2-(6-methyl-2-oxopyridin-1(2H)-yl)ethyl)carbamate (I-488)

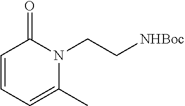 [SMILES: Cc1cccc(=O)n1CCNC(=O)OC(C)(C)C]

To a solution of 6-methylpyridin-2(1H)-one I-487 (cas: 3279-76-3, 5.0 g, 45.87 mmol, 1.0 equiv.), Cs₂CO₃ (29.91 g, 91.74 mmol, 2.0 equiv.) in anhydrous DMF (40 mL) was added tert-butyl (2-bromoethyl)carbamate (cas: 39684-80-5, 15.34 g, 68.81 mmol, 1.5 equiv.). The reaction mixture was stirred at 90° C. for 4 hours. After completion, the reaction mixture was purified by reversed flash column (Mobile Phase: MeCN—H₂O, Gradient: 20-30% H₂O) to afford tert-butyl (2-(6-methyl-2-oxopyridin-1(2H)-yl)ethyl)carbamate, I-488 (3.0 g, 26% yield, 95% purity) as a white solid, MS (ESI, positive ion) m/z: 253.1 (M+1) and byproduct: tert-butyl (2-((6-methylpyridin-2-yl)oxy)ethyl)carbamate (7.0 g, 61% yield, 95% purity) as a white solid, MS (ESI, positive ion) m/z: 253.1 (M+1).

### Step 2: 1-(2-aminoethyl)-6-methylpyridin-2(1H)-one (I-489)

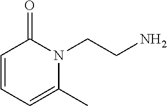

To a solution of tert-butyl (2-(6-methyl-2-oxopyridin-1(2H)-yl)ethyl)carbamate, I-488 (1.5 g, 5.95 mmol, 1.0 equiv.) in DCM (20 mL) was added TFA (10 mL). The reaction mixture was stirred for 2 hours at 25° C. After completion, the reaction mixture was concentrated and the remained TFA was removed by centrifugation to give amine I-489 as a light yellow oil (0.9 g, TFA salt, 101% yield, 90% purity). MS (ESI, positive ion) m/z: 153.1 (M+1).

### Step 3: 2-((2-((2-(6-methyl-2-oxopyridin-1(2H)-yl)ethyl)amino)-2-oxoethyl)thio)acetic acid

### I-490

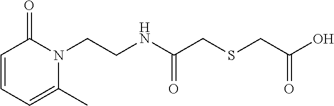 [SMILES: Cc1cccc(=O)n1CCNC(=O)CSCC(=O)O]

The solution of 1-(2-aminoethyl)-6-methylpyridin-2(1H)-one, I-489 (0.9 g, 5.92 mmol, 1.0 equiv.), TEA (1.79 g, 17.76 mmol, 3.0 equiv.), thiodiglycolic anhydride (1.172 g, 8.88 mmol, 1.5 equiv.) in DCM (10 mL) was stirred for 24 hours at 25° C. After completion, the reaction mixture was concentrated. The residue was purified by preparative HPLC (Mobile Phase: ACN-H₂O, Gradient: 5-10% MeCN) to afford carboxylic acid I-490 (119 mg 7% yield, 99.6% purity) as a light yellow solid. MS (ESI, positive ion) m/z: 285.1 (M+1). ¹H NMR (400 MHz, D₂O) δ 7.39 (dd, 1H), 6.34 (dd, 2H), 4.13 (t, 2H), 3.50 (t, 2H), 3.20 (s, 2H), 3.17 (s, 2H), 2.35 (s, 3H).

### The Synthesis of Carboxylic Acid I-494 Involved 3 Steps as Depicted in the Following Scheme 152

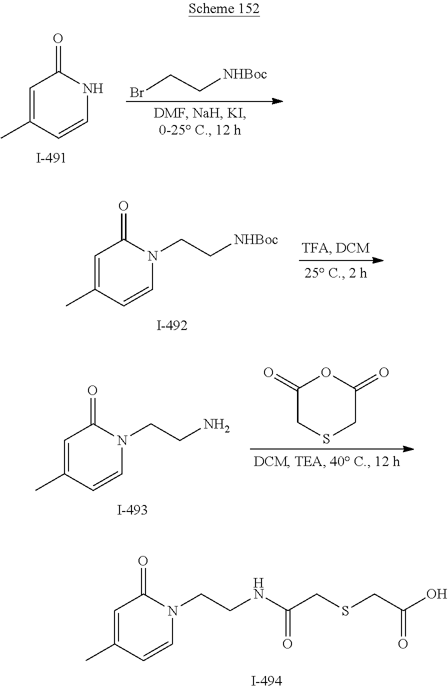

### Step 1: Tert-butyl (2-(4-methyl-2-oxopyridin-1(2H)-yl)ethyl)carbamate (I-492)

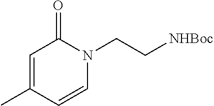 [SMILES: Cc1ccn(CCNC(=O)OC(C)(C)C)c(=O)c1]

A solution of 4-methylpyridin-2(1H)-one, I-491 (cas: 13466-41-6, 5.0 g, 45.87 mmol, 1.0 equiv.), KI (1.522 g, 9.17 mmol, 0.2 equiv.), NaH (60%, 2.20 g, 55.04 mmol, 1.2 equiv.) in DMF (20 ml) was stirred under nitrogen at 0° C. for 1 hour. Then the mixture was added to a solution of tert-butyl (2-bromoethyl)carbamate (cas: 39684-80-5, 15.34 g, 68.80 mmol, 1.5 equiv.) in DMF (30 mL) dropwise. The reaction mixture was stirred at 0° C. for 1 hour and then 25° C. for other 12 hours. LCMS showed ˜50% conversion, at which point H₂O (50 mL) was added and the reaction mixture was extracted with EtOAc (100 mL×3). The organic phase was dried and purified by flash (20%-50% EtOAc with Petroleum ether) to afford tert-butyl (2-(4-methyl-2-oxopyridin-1(2H)-yl)ethyl)carbamate, I-492 (1.3 g, 11% yield, 95% purity) as a white solid, MS (ESI, positive ion) m/z: 253 (M+1) and by product: tert-butyl (2-((4-methylpyridin-2-yl)oxy)ethyl)carbamate (0.2 g, 2% yield, 95% purity), MS (ESI, positive ion) m/z: 253 (M+1).

### Step 2: 1-(2-aminoethyl)-4-methylpyridin-2(1H)-one (I-493)

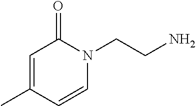

To a solution of tert-butyl (2-(4-methyl-2-oxopyridin-1(2H)-yl)ethyl)carbamate, I-492 (1.3 g, 5.16 mmol, 1.0 equiv.) in DCM (20 mL) was added TFA (10 ml). The solution was stirred for 2 hours at 25° C. After completion, the reaction mixture was concentrated and the remained TFA was removed by centrifugation to afford amine I-493 as a light yellow oil (0.8 g, TFA salt, 102% yield, 90% purity by LCMS). MS (ESI, positive ion) m/z: 153.1 (M+1).

### Step 3: 2-((2-((2-(4-methyl-2-oxopyridin-1(2H)-yl)ethyl)amino)-2-oxoethyl)thio)acetic acid (I-494)

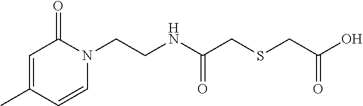 [SMILES: Cc1ccn(CCNC(=O)CSCC(=O)O)c(=O)c1]

The solution of 1-(2-aminoethyl)-4-methylpyridin-2(1H)-one, I-493 (0.8 g, 5.26 mmol, 1.0 equiv.), TEA (1.593 g, 15.78 mmol, 3.0 equiv.), thiodiglycolic anhydride (1.041 g, 7.89 mmol, 1.5 equiv.) in DCM (10 mL) was stirred for 12 hours at 40° C. After completion, the reaction mixture was concentrated. The residue was purified by preparative HPLC (Mobile Phase: ACN—H₂O, Gradient: 5-10% MeCN) to afford I-494 (114.2 mg, 8% yield, 99.6% purity) as a white solid. MS (ESI, positive ion) m/z: 285.1 (M+1). ¹H NMR (400 MHz, D₂O) δ 7.36 (d, 1H), 6.36 (s, 1H), 6.31 (dd, 1H), 4.03-3.97 (m, 2H), 3.52-3.47 (m, 2H), 3.20 (s, 2H), 3.16 (s, 2H), 2.14 (s, 3H).

### The Synthesis of Carboxylic Acid I-498 Involved 3 Steps as Depicted in the Following Scheme 153

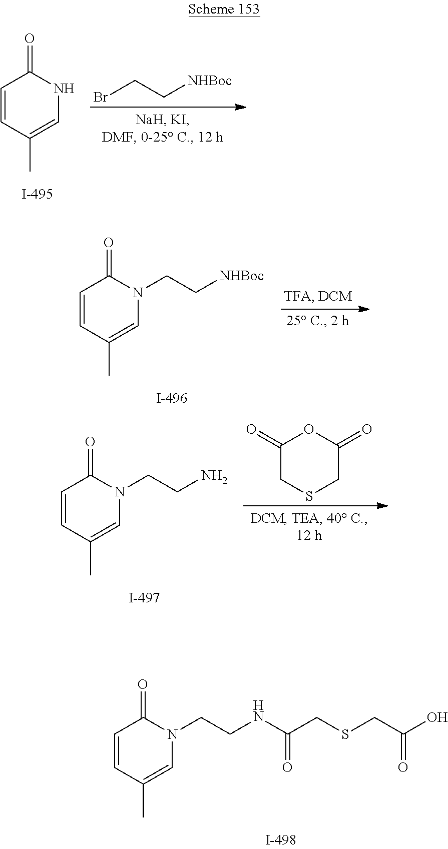

### Step 1: Tert-butyl (2-(5-methyl-2-oxopyridin-1(2H)-yl)ethyl)carbamate (I-496)

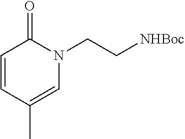 [SMILES: Cc1ccc(=O)n(CCNC(=O)OC(C)(C)C)c1]

A solution of 5-methylpyridin-2(1H)-one, I-495 (cas: 1003-68-5, 5.0 g, 45.87 mmol, 1.0 equiv.), KI (1.522 g, 9.17 mmol, 0.2 equiv.), and NaH (60%, 2.20 g, 55.04 mmol, 1.2 equiv.) in DMF (20 ml) was stirred under nitrogen at 0° C. for 1 hour. Then the reaction mixture was added to a solution of tert-butyl (2-bromoethyl)carbamate (cas: 39684-80-5, 15.34 g, 68.80 mmol, 1.5 equiv.) in DMF (30 mL) dropwise. The reaction mixture was stirred at 0° C. for 1 hour and then 25° C. for 12 hours. LCMS analysis showed ˜50% conversion, at which point H₂O (50 mL) was added and the reaction mixture was extracted with EtOAc (100 mL×3). The organic phase was dried and purified by silica gel chromatography (20%-50% EtOAc with Petroleum ether) to give the product tert-butyl (2-(5-methyl-2-oxopyridin-1(2H)-yl)ethyl)carbamate, I-496 (1.7 g, 15% yield, 95% purity) as a white solid, MS (ESI, positive ion) m/z: 253 (M+1) and byproduct: tert-butyl (2-((5-methylpyridin-2-yl)oxy)ethyl)carbamate (0.4 g, 3% yield, 95% purity), MS (ESI, positive ion) m/z: 253 (M+1).

### Step 2: 1-(2-aminoethyl)-5-methylpyridin-2(1H)-one (I-497)

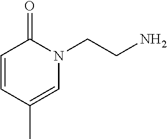

To a solution of tert-butyl (2-(4-methyl-2-oxopyridin-1(2H)-yl)ethyl)carbamate, I-496 (1.7 g, 6.74 mmol, 1.0 equiv.) in DCM (20 mL) was added TFA (10 mL) and the reaction mixture was stirred for 2 hours at 25° C. After completion, the reaction mixture was concentrated and the remaining TFA was removed by centrifugation to give the amine I-497 as a light yellow oil (1.0 g, TFA salt, 103% yield, 90% purity by LCMS). MS (ESI, positive ion) m/z: 153.1 (M+1).

### Step 3: 2-((2-((2-(5-methyl-2-oxopyridin-1(2H)-yl)ethyl)amino)-2-oxoethyl)thio)acetic acid (I-498)

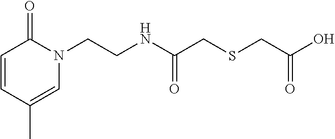 [SMILES: Cc1ccc(=O)n(CCNC(=O)CSCC(=O)O)c1]

A solution of 1-(2-aminoethyl)-5-methylpyridin-2(1H)-one, I-497 (1.0 g, 6.58 mmol, 1.0 equiv.), TEA (1.994 g, 19.74 mmol, 3.0 equiv.), and thiodiglycolic anhydride (1.303 g 9.87 mmol, 1.5 equiv.) was stirred in DCM (10 mL) for 12 hours at 40° C. After completion, the reaction mixture was concentrated. The residue was purified by preparative HPLC (Mobile Phase: ACN—H₂O, Gradient: 5-10% MeCN) to give I-498 (106 mg, 7% yield) as a white solid. MS (ESI, positive ion) m/z: 285.1 (M+1). ¹H NMR (400 MHz, D2O) δ 7.44 (dd, 1H), 7.30 (s, 1H), 6.47 (d, 1H), 4.05-3.98 (m, 2H), 3.56-3.49 (m, 2H), 3.21 (s, 2H), 3.17 (s, 2H), 2.01 (s, 3H).

The synthesis of intermediate I-505 is depicted in Scheme 155:

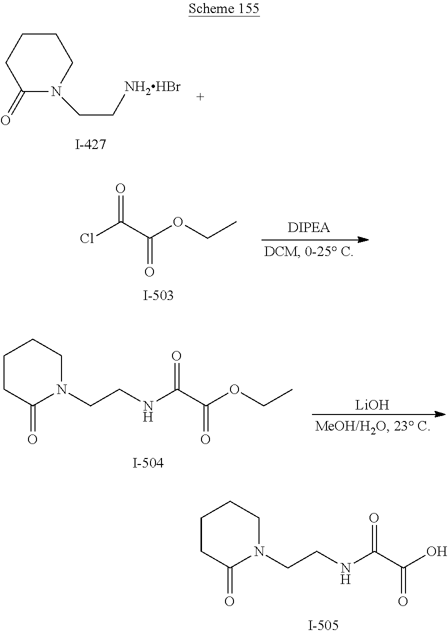

To a solution of amine hydrochloride I-427 (144 mg, 0.645 mmol) and DIPEA (225 μL, 2.0 equiv.) in DCM (5 mL) that had been cooled in an ice-water bath was added acid chloride I-503 (88.1 mg, 1.0 equiv.). The reaction mixture was gradually allowed to warm to ambient temperature and after 3 hours was concentrated to a residue that was purified by preparative HPLC (MeCN/H₂O with 0.1% TFA) to afford ester I-504 (163.8 mg, >99% yield) as a colorless oil.

To a solution of ester I-504 (78 mg, 0.322 mmol) in MeOH (3 mL) and H₂O (1 mL) was added lithium hydroxide (23.1 mg, 3.0 equiv.). The reaction mixture was stirred for 2 hours at ambient temperature at which point analysis by LC-MS indicated that the reaction was complete. The reaction mixture was concentrated and diluted with H₂O (1 mL), then acidified to pH˜1 with 2N HCl. The solution was subjected to purification by preparative HPLC (MeCN/H₂O with 0.1% TFA) to afford carboxylic acid I-505 (56.5 mg, 82% yield) as a white solid. ESI-MS found 215.3, C₉H₁₅N₂O₄ (MH⁺) requires 215.1.

### The Synthesis of Intermediate I-507 is Depicted in Scheme 156

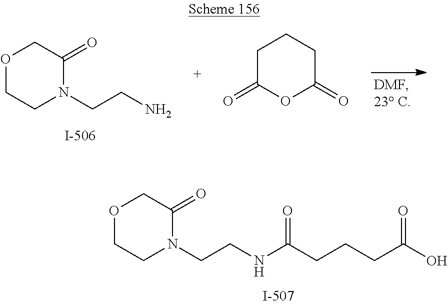 [SMILES: NCCN1CCOCC1=O ; O=C1CCCC(=O)O1 ; O=C(O)CCCC(=O)NCCN1CCOCC1=O]

Amine I-506 (84.2 mg, 0.584 mmol) was treated with Glutaric anhydride (66.6 mg 1.0 equiv.) in DMF (1.1 mL). After 1.5 hours the reaction mixture was subjected to purification by preparative HPLC (MeCN/H₂O with 0.1% TFA) to afford carboxylic acid I-507 (40 mg, 27% yield) as a colorless oil. ESI-MS found 259.2, C₁₁H₁₉N₂O₅ (MH⁺) requires 259.1.

### The Synthesis of Intermediate I-508 is Depicted in Scheme 157

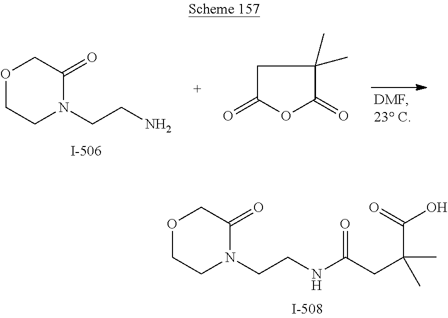

Amine I-506 (96 mg, 0.666 mmol) was treated with 2,2-dimethylsuccinic anhydride (96 mg, 1.1 equiv.) in DMF (1 mL). After 16 hours the reaction mixture was subjected to purification by preparative HPLC (MeCN/H₂O with 0.1% TFA) to afford carboxylic acid I-508 (24.8 mg, 14% yield) as a colorless oil.

### The Synthesis of Intermediate I-510 is Depicted in Scheme 158

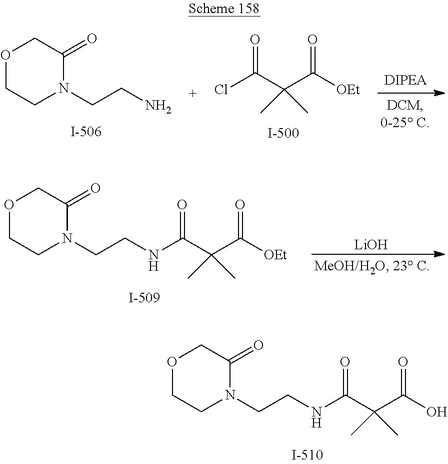 [SMILES: NCCN1CCOCC1=O ; CCOC(=O)C(C)(C)C(=O)Cl ; CCOC(=O)C(C)(C)C(=O)NCCN1CCOCC1=O ; "LiOH" ; CC(C)(C(=O)O)C(=O)NCCN1CCOCC1=O]

To a solution of amine I-506 (169.5 mg, 1.174 mmol) and DIPEA (614 μL, 3.0 equiv.) in DCM (5 mL) that had been cooled in an ice-water bath was added acid chloride I-500 (209.3 mg, 1.0 equiv.). The reaction mixture was allowed to warm to ambient temperature and after 1.5 hours was concentrated to a residue to afford crude ester I-509 as a colorless oil, which was redissolved in MeOH (3 mL). A solution of lithium hydroxide (84.4 mg mg, 3.0 equiv.) in H₂O (1 mL) was added. The reaction mixture was stirred for 17 hours at ambient temperature. The reaction mixture was concentrated and 1M NaHSO₄ and 6N HCl was added to afford a solution of pH˜1.5. Purification by preparative HPLC (MeCN/H₂O with 0.1% TFA) afforded carboxylic acid I-510 (125.3 mg, 41% yield) as a colorless oil. ESI-MS found 259.2, C₁₁H₁₉N₂O₅ (MH⁺) requires 259.1.

### The Synthesis of Intermediate I-513 is Depicted in Scheme 159

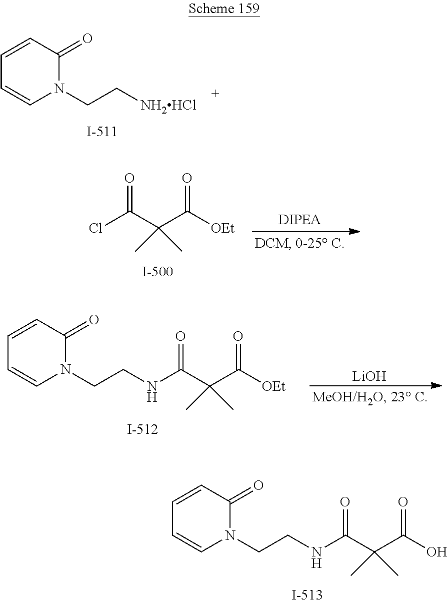

To a solution of amine dihydrochloride I-511 (138.5 mg, 0.656 mmol) and DIPEA (343 μL, 3.0 equiv.) in DCM (4 mL) that had been cooled in an ice-water bath was added acid chloride I-500 (117.2 mg, 1.0 equiv.). The reaction mixture was allowed to warm to ambient temperature and after 1.5 hours was deemed complete by LC-MS analysis. The reaction mixture was concentrated to a residue to afford crude ester I-512 as a colorless oil, which was redissolved in MeOH (2 mL). A solution of lithium hydroxide (47.2 mg mg, 3.0 equiv.) in H₂O (1 mL) was added. The reaction mixture was stirred for 17 hours at ambient temperature. Additional LiOH (47.2 mg mg, 3.0 equiv.) in H₂O (1 mL) was added and stirring was continued for 2 more hours. The reaction mixture was concentrated and 6N HCl was added to afford a solution of PH˜1.5. Purification by preparative HPLC (MeCN/H₂O with 0.1% TFA) afforded carboxylic acid I-513 (136 mg, 82% yield). ESI-MS found 253.2, C₁₂H₁₇N₂O₄ (MH⁺) requires 253.1.

### The Synthesis of Intermediate I-516 is Depicted in Scheme 160

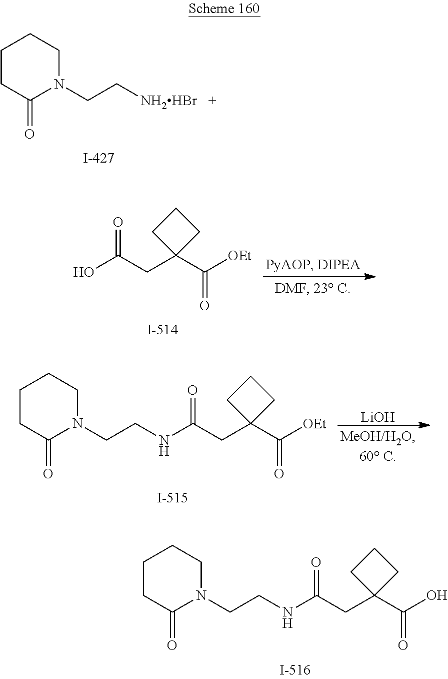

To a solution of amine salt I-427 (62.8 mg, 0.281 mmol), carboxylic acid I-514 (52.4 mg 1.0 equiv.) and DIPEA (147 μL, 3.0 equiv.) in DMF (1 mL) at ambient temperature was added PyAOP (190.7 mg, 1.3 equiv.). The reaction mixture was maintained at ambient temperature for 17 hours and was then purified by preparative HPLC (Mobile Phase: MeCN/H₂O with 0.1% TFA) to afford ester I-515 as a colorless oil (68.7 mg, 79% yield).

To a solution of ester I-515 (68.7 mg, 0.221 mmol) in MeOH (2 mL) and H₂O (1 mL) was added lithium hydroxide (15.9 mg, 3.0 equiv.). The reaction mixture was stirred at ambient temperature for 3 hours, then acidified with 6 N HCl to pH<2, then purified by preparative HPLC (Mobile Phase: MeCN/H₂O with 0.1% TFA) to afford acid I-516 as a white solid (33.9 mg, 54% yield). ESI-MS found 283.3, C₁₄H₂₃N₂O₄ (M+4H)⁴⁺ requires 283.2.

### I-517

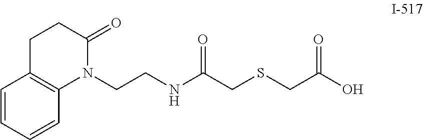 [SMILES: O=C(O)CSCC(=O)NCCN1C(=O)CCc2ccccc21]

Prepared according to GP1. Yield: 32.5 mg (74%). ESI-MS found 323.1, C₁₅H₁₉N₂O₄S (MH⁺) requires 323.1.

### I-518

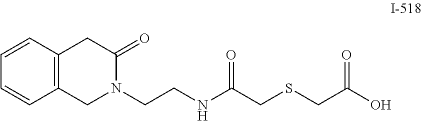 [SMILES: O=C(O)CSCC(=O)NCCN1Cc2ccccc2CC1=O]

Prepared according to GP1. Yield: 18 mg (39%). ESI-MS found 323.3, C₁₅H₁₉N₂O₄S (MH⁺) requires 323.1.

### Synthesis of Intermediate I-552

The synthesis of I-552 involved 5 steps as depicted in the following Scheme 161.

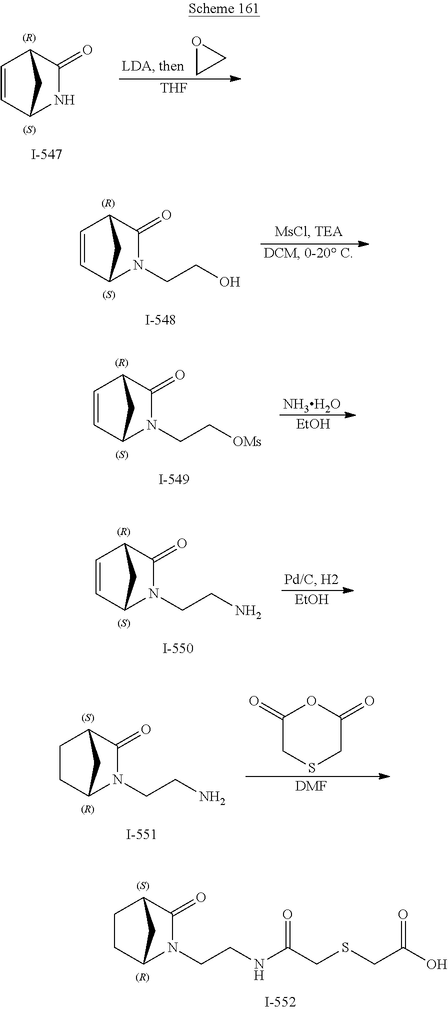

### SUMMARY

The chemical structure of the target compound was confirmed by ¹H NMR and LC-MS with the purity >90% (HPLC 214 & 254 nm).

### Step 1:(1S,4R)-2-(2-hydroxyethyl)-2-azabicyclo[2.2.1]hept-5-en-3-one (I-548)

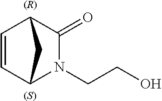

To a solution of (1S,4R)-2-azabicyclo[2.2.1]hept-5-en-3-one, I-547 (2.18 g, 20.0 mmol, CAS: 13031-83-8) in anhydrous THF (30 mL) was added LDA (10 mL, 2M in THF, 1.0 equiv.) at 0° C. The mixture was stirred at 0° C. for 0.5 hours and oxirane (20 mL, 20.0 equiv.) was added. The reaction mixture was allowed to warm to 20° C. and maintained at that temperature for another 16 hours. After the reaction was deemed complete, the pH of the reaction mixture was adjusted to 6 with 1N HCl. The reaction mixture was concentrated to a volume of approximately 5 mL and purified by reversed phase column chromatography (C₁₈ column, 20-35 μm, 100 Å, 80 g, 0.1% v/v Formic acid in MeCN/H₂O) to afford the alcohol I-548 (1.3 g, 43% yield). MS (ESI, pos. ion) m/z: 154.1, (MH⁺).

### Step 2: 2-((1S,4R)-3-oxo-2-azabicyclo[2.2.1]hept-5-en-2-yl)ethyl methanesulfonate (I-549)

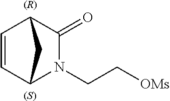

To a solution of methyl (1S,4R)-2-(2-hydroxyethyl)-2-azabicyclo[2.2.1]hept-5-en-3-one, I-548 (1.3 g, 8.50 mmol) and TEA (2.5 g, 3.0 equiv.) in anhydrous THF (20 mL) was added MsCl (1.46 g 1.5 equiv.). The reaction mixture was stirred at 20° C. for 16 hours, then diluted with water (50 mL), and extracted with EtOAc (50 mL). The organic extract was dried over sodium sulfate, and concentrated to afford the crude mesylate I-549 as a yellow oil (1.8 g, 92% yield), which was used for the next step. MS (ESI, pos. ion) m/z: 232.0, (MH⁺).

### Step 3: (1S,4R)-2-(2-aminoethyl)-2-azabicyclo[2.2.1]hept-5-en-3-one (I-550)

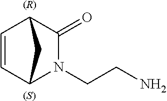

To a solution of crude 2-((1S,4R)-3-oxo-2-azabicyclo[2.2.1]hept-5-en-2-yl)ethyl methanesulfonate, I-549 (1.8 g, 7.79 mmol) in EtOH (20 mL) was added concentrated aqueous NH₃ (25-28% solution, 5 mL). The mixture was stirred at 100° C. for 0.5 hours. After completion, the reaction mixture was concentrated to ca. 5 mL and purified by reversed phase column chromatography (0.1% Formic acid in MeCN/H₂O) to afford the desired amine I-550 as a colorless syrup (550 mg, 47% yield). MS (ESI, pos. ion) m/z: 153.1, (MH⁺).

### Step 4: (1R,4S)-2-(2-aminoethyl)-2-azabicyclo[2.2.1]heptan-3-one (I-551)

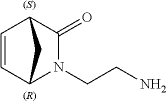

A slurry of (1S,4R)-2-(2-aminoethyl)-2-azabicyclo[2.2.1]hept-5-en-3-one, I-550 (200 mg, 1.32 mmol) and Pd/C (10%, 100 mg) in EtOH (6 mL) was stirred under H₂ (1 atm) at 20° C. for 16 hours. The reaction mixture was then filtered, and the filtrate was concentrated to afford the desired amine I-551 as a colorless syrup (200 mg, 98% yield). MS (ESI, pos. ion) m/z: 155.1, (MH⁺).

### Step 5: 2-((2-oxo-2-((2-((1R,4S)-3-oxo-2-azabicyclo[2.2.1]heptan-2-yl)ethyl)amino)ethyl)thio)acetic acid (I-552)

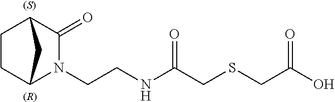

To a solution of (1R,4S)-2-(2-aminoethyl)-2-azabicyclo[2.2.1]heptan-3-one (200 mg, 1.30 mmol) in DMF (3 mL) was added thiodiglycolic anhydride (343 mg, 2.0 equiv.), and the reaction mixture was stirred at 40° C. for 16 hours, then purified by reversed phase column chromatography (0.1% Formic acid in MeCN/H₂O) to afford acid I-552 as a colorless syrup (160 mg, 42% yield). MS (ESI, pos. ion) m/z: 287.1, (MH⁺). 1H NMR (400 MHz, D₂O) δ 4.04 (s, 1H), 3.42-3.54 (m, 2H), 3.31-3.38 (m, 5H), 3.03-3.09 (m, 1H), 2.80-2.81 (m, 1H), 1.90-1.98 (m, 1H), 1.80-1.86 (m, 2H), 1.59-1.65 (m, 1H), 1.39-1.46 (m, 2H).

### Synthesis of Intermediate I-557

The synthesis of intermediate I-557 involved 4 steps as described in the following Scheme 162.

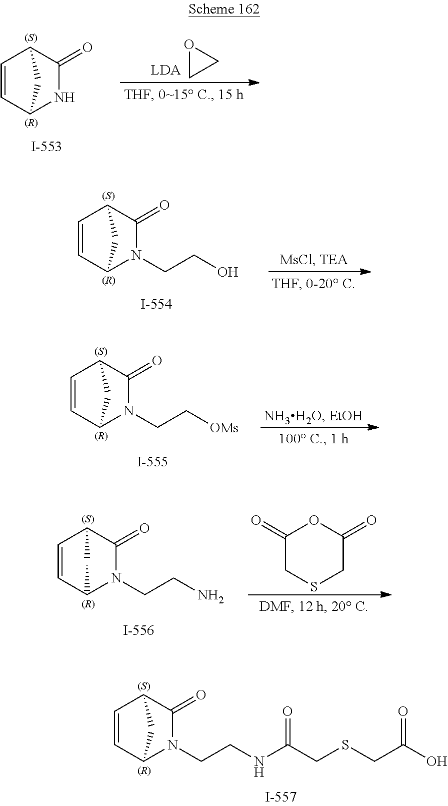

### SUMMARY

The chemical structure of the target compound was confirmed by 1HNMR and LC-MS with the purity >95% (HPLC 214 & 254 nm).

### Step 1: (1R,4S)-2-(2-hydroxyethyl)-2-azabicyclo[2.2.1]hept-5-en-3-one (I-554)

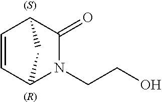

To a solution of (1R,4S)-2-azabicyclo[2.2.1]hept-5-en-3-one (3 g, CAS: 79200-56-9, 27.5 mmol) in THF (50 mL) was added LDA (2M in THF, 13.8 mL, 1.0 equiv.) at 25° C. The reaction mixture was stirred for 1 h at 25° C. and then oxirane (cas: 75-21-8, 20 mL, 17.6 g, 0.4 mol, 14.5 equiv.) was added. The reaction mixture was stirred at 25° C. for 48 hours. When the reaction was deemed complete by LC-MS analysis, the pH of the reaction mixture was adjusted to 6-7 with HCl (1M). The reaction mixture was concentrated to a residue, which was purified by reversed phase column chromatography (0.1% Formic acid in MeCN/H₂O) to afford (1R,4S)-2-(2-hydroxyethyl)-2-azabicyclo[2.2.1]hept-5-en-3-one, I-554 (2.8 g, 67% yield) as a yellow oil. MS (ESI, pos. ion) found m/z: 154.1 (MH⁺).

### Step 2: 2-((1R,4S)-3-oxo-2-azabicyclo[2.2.1]hept-5-en-2-yl)ethyl methanesulfonate(I-555)

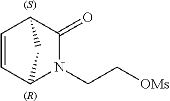

To a solution of (1R,4S)-2-(2-hydroxyethyl)-2-azabicyclo[2.2.1]hept-5-en-3-one (2.8 g, 18.3 mmol) in THF (50 mL) was added TEA (5.5 g, 3.0 equiv.) and MsCl (2.08 g, 1.0 equiv.) at 25° C. The reaction mixture was stirred at 25° C. for 2 hours. When the reaction was deemed complete by LC-MS analysis, the reaction mixture diluted with water (50 mL), and extracted with EtOAc (50 mL). The organic extract was washed with water (2×40 mL), brine (2×30 mL) and dried over anhydrous Na₂SO₄, then concentrated to afford the crude mesylate I-555 (3.0 g 71% yield) as a yellow solid, which was used for next step without further purification. MS (ESI, neg. ion) m/z: 232.0 (M+1).

### Step 3: (1R,4S)-2-(2-aminoethyl)-2-azabicyclo[2.2.1]hept-5-en-3-one (I-556)

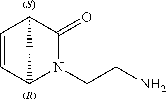

To a solution of crude mesylate I-555 (3.0 g, 12.9 mmol) in EtOH (20 mL) was added concentrated aqueous ammonia (25-28% solution, 5 mL), and the reaction mixture was stirred at 100° C. for 1 hour, then concentrated to ca. 5 mL and purified by reversed phase column chromatography (0.1% Formic acid in MeCN/H₂O) to afford the desired amine I-556 (1.8 g, 95% yield) as a colorless syrup. MS (ESI, pos. ion) m/z: 153.1, (MH⁺).

### Step 4: 2-((2-oxo-2-((2-((1R,4S)-3-oxo-2-azabicyclo[2.2.1]hept-5-en-2-yl)ethyl)amino)ethyl)thio)acetic acid. (I-557)

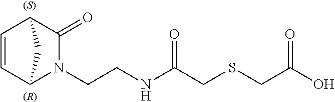

To a solution of (1R,4S)-2-(2-aminoethyl)-2-azabicyclo[2.2.1]hept-5-en-3-one (1.4 g, 9.1 mmol) in DMF (3 mL) was added thiodiglycolic anhydride (2.4 g, 2.0 equiv.), and the reaction mixture was stirred at 40° C. for 12 hours. The reaction mixture was purified by Preparatory HPLC (0.1% Formic acid in MeCN/H₂O) to afford carboxylic acid I-557 (33.1 mg, 1% yield) a white solid. MS (ESI, pos. ion) m/z: 285.1, (MH⁺). ¹H NMR (400 MHz, D₂O) δ6.18-6.24 (m, 1H), 6.01-6.05 (m, 1H), 5.59-5.61 (m, 0.5H), 5.25-5.27 (m, 0.5H), 4.17-4.24 (m, 0.5H), 3.89-3.95 (m, 0.5H), 3.56-3.82 (m, 4H), 3.37-3.54 (m, 4H), 2.44-2.61 (m, 2H).

### Synthesis of Intermediate I-559

The synthesis of intermediate I-559 from previously described intermediate I-556 involved 2 steps as depicted in the following Scheme 163.

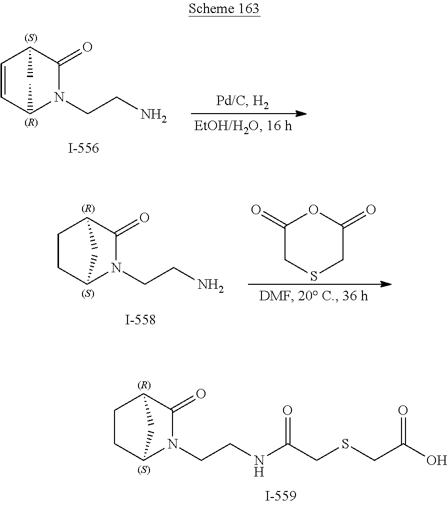

### SUMMARY

The chemical structure of the target compound was confirmed by 1HNMR and LC-MS with the purity >90% (HPLC 214 & 254 nm).

### Step 1:(1S,4R)-2-(2-aminoethyl)-2-azabicyclo[2.2.1]heptan-3-one, I-558

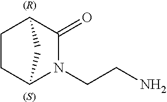

A solution of amine I-556 (1.0 g, 6.6 mmol) and 10% Pd/C (300 mg) in EtOH (10 mL) was stirred under H₂ (1 atm) at 20° C. for 16 hours. The reaction mixture was then filtered, and the filtrate was concentrated to afford the amine I-558 (1.0 g, 98% yield) as a colorless syrup. MS (ESI, pos. ion) m/z: 155.1 (M+1).

### Step 2: 2-((2-oxo-2-((2-((1S,4R)-3-oxo-2-azabicyclo[2.2.1]heptan-2-yl)ethyl)amino)ethyl)thio)acetic acid (I-559)

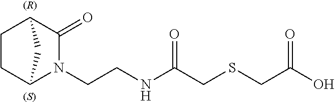

To a solution of amine I-558 (1.0 g, 6.6 mmol) in DMF (10 mL) was added thiodiglycolic anhydride (1.7 g, 2.0 equiv.). The reaction mixture was stirred at 40° C. for 36 hours, then purified directly by Preparative HPLC (0.1% TFA in MeCN/H₂O) to afford carboxylic acid I-559 (46.1 mg, 3% yield) as a colorless syrup. MS (ESI, pos. ion) m/z: 287.1, (MH⁺). ¹H NMR (400 MHz, D₂O) δ4.06 (s, 1H), 3.46-3.54 (m, 2H), 3.31-3.38 (m, 5H), 3.05-3.11 (m, 1H), 2.82 (s, 1H), 1.94-1.99 (m, 1H), 1.81-1.88 (m, 2H), 1.59-1.65 (m, 1H), 1.34-1.48 (m, 2H).

### Synthesis of Intermediate I-571

The synthesis of I-571 involved 9 steps as described in the following Scheme 164.

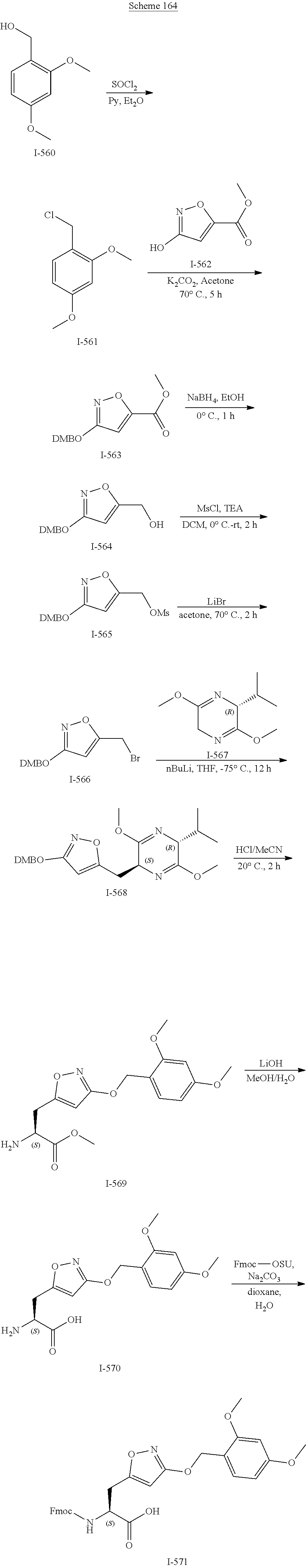

### SUMMARY

The chemical structure of the target compound was confirmed by ¹HNMR and LC-MS with the purity >95% (HPLC 214 & 254 nm).

### Step 1: 1-(chloromethyl)-2,4-dimethoxybenzene. (I-561)

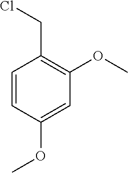

To a solution of (2,4-dimethoxyphenyl)methanol, I-560 (10 g, 59.5 mmol) in Et₂O (100 mL) was added pyridine (4.65 g, 1.0 equiv.), and SOCl₂ (7.02 g, 1.0 equiv.) at 0° C. The reaction mixture was stirred at 0° C. for 2 hours, then poured into ice water (120 mL) and the layers were separated. The aqueous layer was extracted with diethyl ether (2×60 mL) and the combined organic extracts were washed with ice water (60 mL) and a solution of 5:1 saturated aqueous sodium chloride: saturated aqueous sodium bicarbonate (2×60 mL), dried over anhydrous sodium sulfate, filtered, and concentrated to ˜50 mL of solution. The crude solution was used directly in next step.

### Step 2: Methyl 3-((3,4-dimethylbenzyl)oxy)isoxazole-5-carboxylate (I-563)

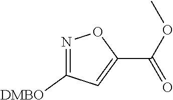

To a solution of 1-chloromethyl)-2,4-dimethoxybenzene, I-561 (20 g, 107 mmol, 5.0 equiv.) in acetone (200 mL) was added methyl 3-hydroxyisoxazole-5-carboxylate, I-562 (3 g, 21.5 mmol) and K₂CO₃ (8.9 g, 3.0 equiv.). The reaction mixture was stirred at 70° C. for 19 hours. When the reaction was deemed complete by LC-MS analysis, the reaction mixture was concentrated under reduced pressure. The crude product was purified by column chromatography (silica gel, Petroleum/EtOAc=1:1) to afford methyl 3-((3,4-dimethylbenzyl)oxy)isoxazole-5-carboxylate, I-563 (2.0 g, 32% yield, as a yellow oil).

### Step 3: (3-((3,4-dimethylbenzyl)oxy)isoxazol-5-yl)methanol. (I-564)

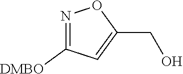

To a solution of methyl 3-[(3,4-dimethylphenyl)methoxy]-1,2-oxazole-5-carboxylate, I-563 (1.1 g, 4.2 mmol) in EtOH (10 mL) was added NaBH₄ (245 mg, 1.5 equiv.) at 0° C. The reaction mixture was allowed to warm to 25° C. and stirred for 2 hours. After completion, the reaction mixture was quenched with H₂O (5 mL), and extracted with DCM (20 mL). The organic extract was dried (Na₂SO₄), concentrated under reduced pressure and the residue obtained was purified by silica gel chromatography (Petroleum ether:EtOAc=1:1) to afford (3-((3,4-dimethylbenzyl)oxy)isoxazol-5-yl) methanol, I-564 (0.9 g, quantitative yield) as a yellow oil. MS (ESI, pos. ion) m/z: 288.1 (M+Na).

### Step 4: (3-((3,4-dimethylbenzyl)oxy)isoxazol-5-yl)methyl methanesulfonate. (I-565)

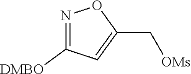

To a solution of {3-[(3,4-dimethylphenyl)methoxy]-1,2-oxazol-5-yl}methanol, I-564 (300 mg, 1.287 mmol) in DCM (5 mL) was added MsCl (440 mg, 3.0 equiv.) and TEA (258 mg, 2.0 equiv.) at 0° C. The reaction mixture was allowed to warm to 25° C. and stirred for 2 hours. After completion, the reaction mixture was quenched with H₂O (15 mL), and extracted with DCM (50 mL). The organic extract was dried (Na₂SO₄), concentrated under reduced pressure and the residue obtained was purified by silica gel chromatography (Petroleum ether:EtOAc=2:1) to afford (3-((3,4-dimethylbenzyl) oxy)isoxazol-5-yl)methyl methane sulfonate, I-565 (300 mg, 77% yield) as a yellow oil. MS (ESI, pos. ion) m/z: 366.0 (M+Na).

### Step 5: 5-(bromomethyl)-3-((3,4-dimethylbenzyl)oxy)isoxazole. (I-566)

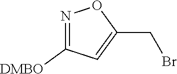

To a solution of {3-[(3,4-dimethylphenyl)methoxy]-1,2-oxazol-5-yl}methyl methane sulfonate, I-565 (2.9 g, 9.3 mmol) in acetone (50 mL) was added LiBr (2.4 g, 3.0 equiv.). The reaction mixture was stirred at 60° C. for 3 hours. When the reaction was deemed complete by LC-MS analysis, the reaction mixture was concentrated under reduced pressure to a residue, which was purified by silica gel column chromatography (DCM:MeOH=20:1) to afford 5-(bromomethyl)-3-((3,4-dimethylbenzyl)oxy)isoxazole, I-566 (2.5 g, 90% yield) as a yellow oil. MS (ESI, pos. ion) m/z: 350.0 (M+Na).

### Step 6: 3-((3,4-dimethylbenzyl)oxy)-5-(((2S,5R)-5-isopropyl-3,6-dimethoxy-2,5-dihydropyrazin-2-yl)methyl)isoxazole. (I-568)

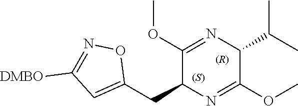

To a solution of (3R)-3-isopropyl-2,5-dimethoxy-3,6-dihydropyrazine, I-567 (46 mg, CAS: 109838-95-9, 0.25 mmol) in THF (5 mL) was added n-BuLi (2.5M in hexane, 0.1 ml, 1.0 equiv.) at −78° C. under N₂. After 30 minutes, 5-(bromomethyl)-3-[(3,4-dimethylphenyl)methoxy]-1,2-oxazole, I-566 (50 mg, 0.7 equiv.) was added. The flask was evacuated and purged again with nitrogen. The resulting mixture was slowly warmed to room temperature over 18 hours. When the reaction was deemed complete by LC-MS analysis, the reaction mixture was quenched with water (2 mL) and extracted with EtOAc (2×10 mL). The combined organic extracts were washed with brine, dried over Na₂SO₄ filtered and concentrated under reduced pressure. The residue was purified by silica gel chromatography (Petroleum:EtOAc=2:1) to afford 3-((3,4-dimethylbenzyl)oxy)-5-(((2S,5R)-5-isopropyl-3,6-dimethoxy-2,5-dihydropyrazin-2-yl)methyl) isoxazole, I-568 (20 mg, 30% yield) as a yellow oil. MS (ESI, pos. ion) m/z: 432.2 (MH⁺).

### Step 7: Methyl(S)-2-amino-3-(3-((2,4-dimethoxybenzyl)oxy)isoxazol-5-yl) propanoate (I-569)

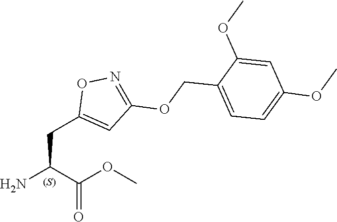

A mixture of (2S,5R)-2-({3-[3,4-dimethylphenyl)methoxy]-1,2-oxazol-5-yl}methyl)-5-isopropyl-3,6-dimethoxy-2,5-dihydropyrazine, I-568 (30 mg, 0.05 mmol) in 1N HCl (1 mL) and MeCN (5 mL) was stirred under 30° C. for 3 hours. The solvent was then removed under reduced pressure to afford the crude methyl (S)-2-amino-3-(3-((2,4-dimethoxybenzyl)oxy) isoxazol-5-yl) propanoate, I-569 (20 mg, 86% yield) as a yellow oil, which was used in the next step without any purification. MS (ESI, pos. ion) m/z: 337.1 (MH⁺).

### Step 8: (S)-2-amino-3-(3-((2,4-dimethoxybenzyl)oxy)isoxazol-5-yl) propanoic acid. (I-570)

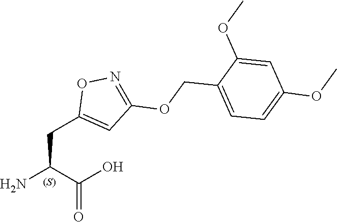

To a stirred solution of methyl (S)-2-amino-3-{3-[(2,4-dimethoxyphenyl)methoxy]-1,2-oxazol-5-yl}propanoate, I-569 (1.2 g, 3.57 mmol) in MeOH (20 mL) was added LiOH (246 mg, 3.0 equiv.) in H₂O (20 mL) at 0° C. The reaction mixture was allowed to warm to room temperature over 2 hours. The reaction mixture was then concentrated to a residue, which was purified by reverse phase HPLC (MeCN/H₂O with 0.5% formic acid) to afford (S)-2-amino-3-(3-((2,4-dimethoxybenzyl)oxy)isoxazol-5-yl) propanoic acid, I-570 (300 mg, 25% yield) as a yellow solid. MS (ESI, pos. ion) m/z: 323.1 (MH⁺).

### Step 8: (S)-2-amino-3-(3-((2,4-dimethoxybenzyl)oxy)isoxazol-5-yl) propanoic acid (I-571)

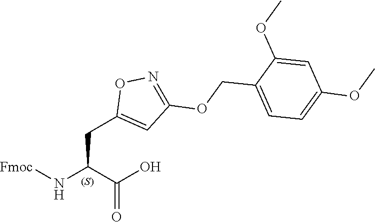

To a solution of (S)-2-amino-3-{3-[(2,4-dimethoxyphenyl)methoxy]-1,2-oxazol-5-yl}propanoic acid, I-570 (300 mg, 0.93 mmol) in dioxane (10 mL) and H₂O (10 mL) was added Fmoc-OSu (340 mg, 1.3 equiv.) and Na₂CO₃ (197 mg, 2.0 equiv.). The mixture was stirred for 2 hours at 20° C. When the reaction was deemed complete by LC-MS analysis, the pH of the reaction mixture was adjusted to 2 using IN HCl. The reaction mixture was then extracted with DCM (30 mL), and the organic extract was dried (Na₂SO₄) and concentrated under reduced pressure. The residue obtained was purified by preparatory TLC (DCM:MeOH=10:1) to afford the desired carboxylic acid, I-571 (200 mg, 40% yield) as a white solid. MS (ESI, pos. ion) m/z: 545.2 (MH⁺). ¹H NMR (400 MHz, DMSO-d6) δ=7.89 (d, J-7.5, 2H), 7.65 (d, J=4.5, 2H), 7.41 (t, J=7.4, 2H), 7.37-7.23 (m, 2H), 6.97 (d, J=8.4, 1H), 6.53 (d, J=2.1, 1H), 6.41 (dd, J=8.3, 2.3, 1H), 5.56 (s, 1H), 4.76 (s, 2H), 4.21 (dd, J-15.1, 8.1, 3H), 3.86 (s, 1H), 3.74 (s, 3H), 3.69 (s, 3H), 2.96 (d, J=11.9, 1H), 2.84 (d, J=6.9, 1H).

### D2. Additional Examples of Compounds Described in this Invention

### Example 197: Compound 197

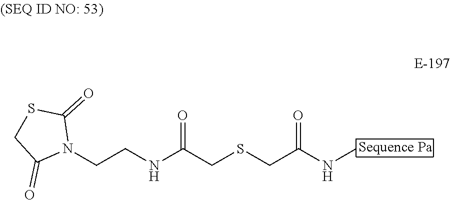 [SMILES: CNC(=O)CSCC(=O)NCCN1C(=O)CSC1=O]

Peptide E-197 was prepared from 31.7 mg of the corresponding sequence immobilized on Rink amide resin (estimated loading 0.19 mmol/g) using I-34 and GP5 to afford 1.0 mg of E-197 as a white solid. ESI-MS found 1160.8, C₂₁₄H₃₂₇S₂N₄₇O₆₄ (M+4H)⁴⁺ requires 1160.8.

### Example 198: Compound 198

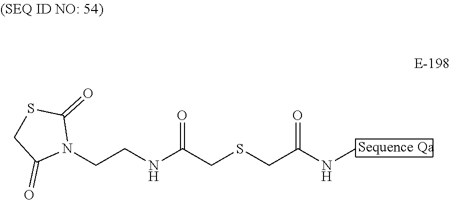 [SMILES: CNC(=O)CSCC(=O)NCCN1C(=O)CSC1=O]

Peptide E-198 was prepared from 64.4 mg of the corresponding sequence immobilized on Rink amide resin (estimated loading 0.11 mmol/g) using I-9 and GP4 to afford 3.2 mg of E-198 as a white solid. ESI-MS found 1613.4, C₂₂₀H₃₃₅S₂N₅₂O₆₇ (M+3H)³⁺ requires 1613.8.

### Example 199: Compound 199

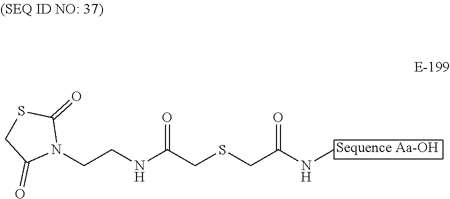 [SMILES: CNC(=O)CSCC(=O)NCCN1C(=O)CSC1=O]

Peptide E-199 was prepared from 138.8 mg of the corresponding sequence immobilized on chlorotrityl resin (estimated loading 0.18 mmol/g) using I-9 and GP4 to afford 6.5 mg of E-199 as a white solid. ESI-MS found 1542.9, C₂₁₂H₃₁₉S₂N₄₆O₆₆ (M+3H)³⁺ requires 1543.1.

### Example 200: Compound 200

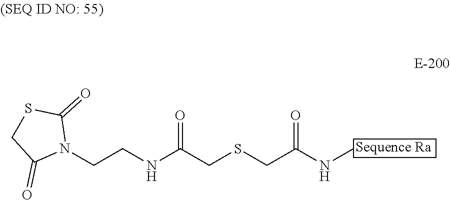 [SMILES: CNC(=O)CSCC(=O)NCCN1C(=O)CSC1=O]

Peptide E-200 was prepared from 158 mg of the corresponding sequence immobilized on Rink amide resin (estimated loading 0.16 mmol/g) using I-9 and GP4 to afford 12 mg of E-200 as a white solid. ESI-MS found 1556.2, C₂₁₄H₃₂₅S₂N₄₈O₆₅ (M+3H)³⁺ requires 1557.1.

### Example 201: Compound 201

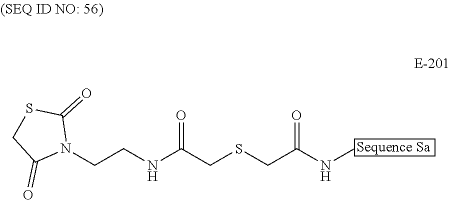 [SMILES: CNC(=O)CSCC(=O)NCCN1C(=O)CSC1=O]

Peptide E-201 was prepared from 184 mg of the corresponding sequence immobilized on Rink amide resin (estimated loading 0.16 mmol/g) using I-9 and GP4 to afford 14 mg of E-201 as a white solid. ESI-MS found 1532.4, C₂₀₉H₃₂₃S₂N₄₈O₆₄ (M+3H)³⁺ requires 1531.1.

### Example 202: Compound 202

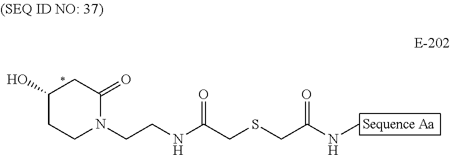 [SMILES: CNC(=O)CSCC(=O)NCCN1CC[C@H](O)CC1=O]

Peptide E-202 was prepared from 30 mg of resin R-1 (estimated loading 0.125 mmol/g) using I-443 (*absolute configuration arbitrarily assigned) and GP4 to afford 1.9 mg of E-202 as a white solid. ESI-MS found 1157.3, C₂₁₄H₃₂₇SN₄₇O₆₅ (M+4H)⁴⁺ requires 1156.8.

### Example 203: Compound 203

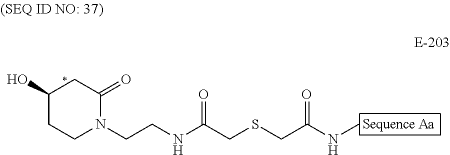 [SMILES: CNC(=O)CSCC(=O)NCCN1CC[C@@H](O)CC1=O]

Peptide E-203 was prepared from 30 mg of resin R-1 (estimated loading 0.125 mmol/g) using I-439 (*absolute configuration arbitrarily assigned) and GP4 to afford 1.2 mg of E-203 as a white solid. ESI-MS found 1157.2, C₂₁₄H₃₂₇SN₄₇O₆₅ (M+4H)⁴⁺ requires 1156.8.

### Example 204: Compound 204

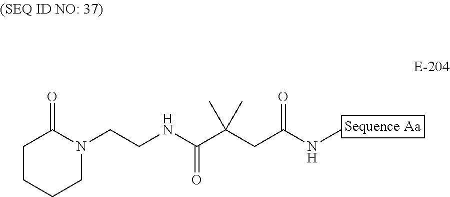 [SMILES: CNC(=O)CC(C)(C)C(=O)NCCN1CCCCC1=O]

Peptide E-204 was prepared from 39.6 mg of resin R-1 (estimated loading 0.125 mmol/g) using I-430 and GP4 to afford 3.2 mg of E-204 as a white solid. ESI-MS found 1152.3, C₂₁₆H₃₃₁N₄₇O₆₄ (M+4H)⁴⁺ requires 1151.8.

### Example 205: Compound 208

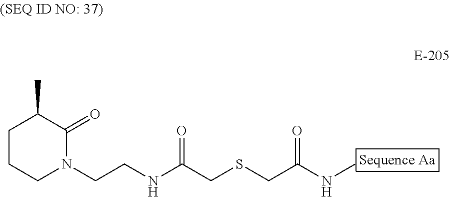 [SMILES: CNC(=O)CSCC(=O)NCCN1CCC[C@@H](C)C1=O]

Peptide E-205 was prepared from 46.6 mg of resin R-1 (estimated loading 0.125 mmol/g) using I-451 and GP4 to afford 6.4 mg of E-205 as a white solid. ESI-MS found 1156.9, C₂₁₅H₃₂₉SN₄₇O₆₄ (M+4H)⁴⁺ requires 1156.3.

### Example 206: Compound 207

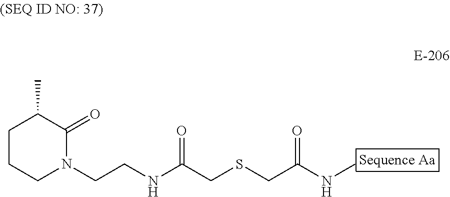 [SMILES: CNC(=O)CSCC(=O)NCCN1CCC[C@H](C)C1=O]

Peptide E-206 was prepared from 44.4 mg of resin R-1 (estimated loading 0.125 mmol/g) using I-454 and GP4 to afford 5.9 mg of E-206 as a white solid. ESI-MS found 1156.9, C₂₁₅H₃₂₉SN₄₇O₆₄ (M+4H)⁴⁺ requires 1156.3.

### Example 207: Compound 205

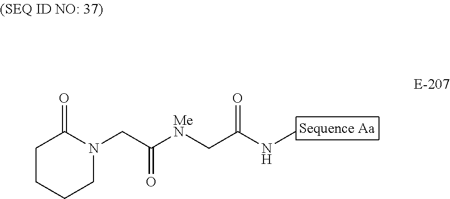 [SMILES: CNC(=O)CN(C)C(=O)CN1CCCCC1=O]

Peptide E-207 was prepared from 37 mg of resin R-1 (estimated loading 0.125 mmol/g) using I-458 and GP4 to afford 4.8 mg of E-207 as a white solid. ESI-MS found 1141.8, C₂₁₃H₃₂₅N₄₇O₆₄ (M+4H)⁴⁺ requires 1141.3.

### Example 208: Compound 206

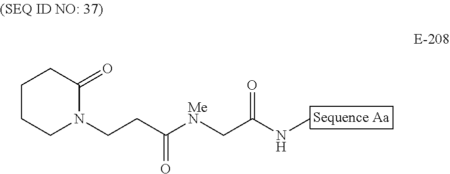 [SMILES: CNC(=O)CN(C)C(=O)CCN1CCCCC1=O]

Peptide E-208 was prepared from 40.4 mg of resin R-1 (estimated loading 0.125 mmol/g) using I-461 and GP4 to afford 7 mg of E-208 as a white solid. ESI-MS found 1145.3, C₂₁₄H₃₂₇N₄₇O₆₄ (M+4H)⁴⁺ requires 1144.8.

### Example 209: Compound 209

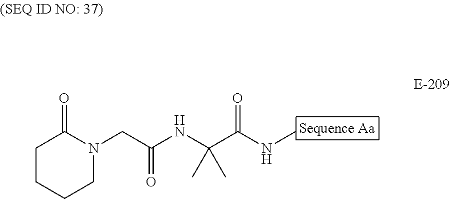 [SMILES: CNC(=O)C(C)(C)NC(=O)CN1CCCCC1=O]

Peptide E-209 was prepared from 44.2 mg of resin R-1 (estimated loading 0.125 mmol/g) using I-464 and GP4 to afford 2.2 mg of E-209 as a white solid. ESI-MS found 1145.3, C₂₁₄H₃₂₇N₄₇O₆₄ (M+4H)⁴⁺ requires 1144.8.

### Example 210: Compound 210

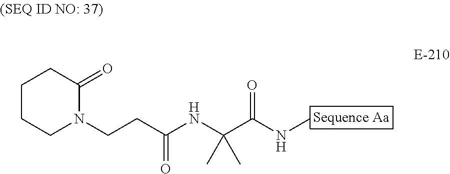 [SMILES: CNC(=O)C(C)(C)NC(=O)CCN1CCCCC1=O]

Peptide E-210 was prepared from 43.2 mg of resin R-1 (estimated loading 0.125 mmol/g) using I-466 and GP4 to afford 1.5 mg of E-210 as a white solid. ESI-MS found 1148.9, C₂₁₅H₃₂₉N₄₇O₆₄ (M+4H)⁴⁺ requires 1148.3.

### Example 211: Compound 211

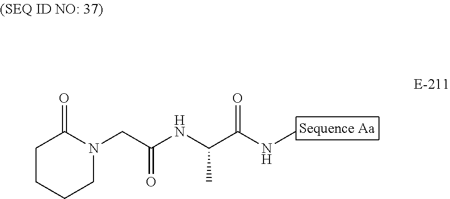 [SMILES: CNC(=O)[C@H](C)NC(=O)CN1CCCCC1=O]

Peptide E-211 was prepared from 48.2 mg of resin R-1 (estimated loading 0.125 mmol/g) using I-469 and GP4 to afford 1.5 mg of E-211 as a white solid. ESI-MS found 1141.8, C₂₁₃H₃₂₅N₄₇O₆₄ (M+4H)⁴⁺ requires 1141.3.

### Example 212: Compound 212

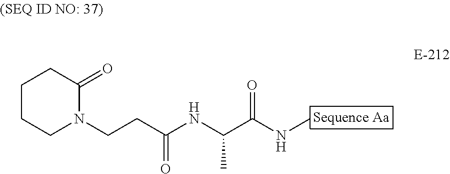 [SMILES: CNC(=O)[C@H](C)NC(=O)CCN1CCCCC1=O]

Peptide E-212 was prepared from 44.2 mg of resin R-1 (estimated loading 0.125 mmol/g) using I-471 and GP4 to afford 3.8 mg of E-212 as a white solid. ESI-MS found 1145.3, C₂₁₄H₃₂₇N₄₇O₆₄ (M+4H)⁴⁺ requires 1144.8.

### Example 213: Compound 213

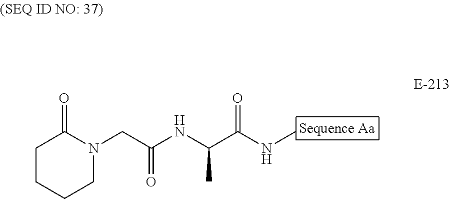 [SMILES: CNC(=O)[C@@H](C)NC(=O)CN1CCCCC1=O]

Peptide E-213 was prepared from 44.1 mg of resin R-1 (estimated loading 0.18 mmol/g) using I-474 and GP4 to afford 4.3 mg of E-213 as a white solid. ESI-MS found 1141.9, C₂₁₃H₃₂₅N₄₇O₆₄ (M+4H)⁴⁺ requires 1141.3.

### Example 214: Compound 214

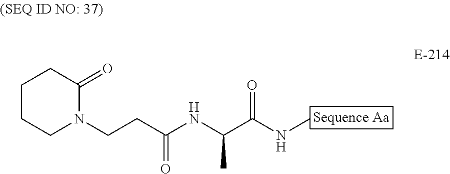 [SMILES: CNC(=O)[C@@H](C)NC(=O)CCN1CCCCC1=O]

Peptide E-214 was prepared from 45.9 mg of resin R-1 (estimated loading 0.125 mmol/g) using I-476 and GP4 to afford 3.0 mg of E-214 as a white solid. ESI-MS found 1145.4, C₂₁₄H₃₂₇N₄₇O₆₄ (M+4H)⁴⁺ requires 1144.8.

### Example 215: Compound 217

### The Synthesis of Example E-215 is Depicted in Scheme 167

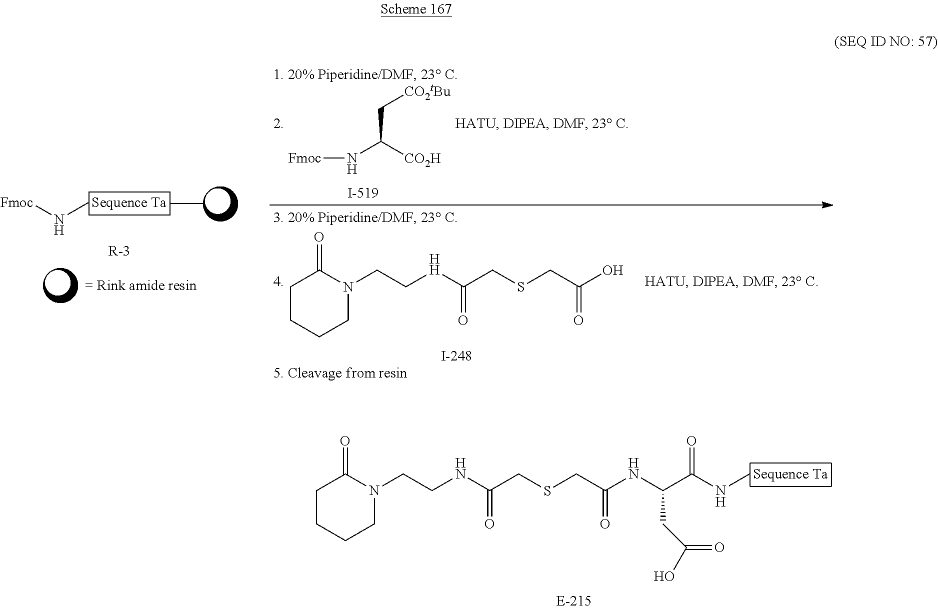

To Rink amide resin R-3 (0.17 mmol/g, 60 mg, 0.01 mmol) in a 3 mL polypropylene tube with an end-cap was added 20% v/v piperidine/DMF (2 mL). The tube was capped, agitated at ambient temperature for 20 minutes, and then drained. The resin was washed with DMF (5×3 mL). A solution of carboxylic acid I-519 (25.2 mg, 6.0 equiv.) in DMF (1.2 mL) was added, followed by DIPEA (27 μL, 15.0 equiv.) and HATU (34.9 mg, 9.0 equiv.). The reaction mixture was agitated at ambient temperature for 5 hours, then drained and the resin was washed with DMF (5×3 mL) and DCM (5×3 mL).

20% v/v piperidine/DMF (2 mL) was added to the resin. The tube was capped, agitated at ambient temperature for 20 minutes, and then drained. The resin was washed with DMF (5×3 mL). A solution of carboxylic acid I-248 (16.7 mg, 6.0 equiv.) in DMF (1.2 mL) was added, followed by DIPEA (27 μL, 15.0 equiv.) and HATU (34.9 mg, 9.0 equiv.). The reaction mixture was agitated at ambient temperature for 16 hours, then drained and the resin was washed with DMF (5×3 mL) and DCM (5×3 mL). The peptide was cleaved from the resin using 1 mL of cleavage reagent (88:2:5:5 v/v/v/v TFA/TIS/PhOH/H₂O) for 1 hour. The resin was filtered and washed with TFA (2×0.5 mL). The combined filtrate and washes were concentrated under reduced pressure to afford a residue, which was triturated with Et₂O (2 mL) to precipitate the peptide. The peptide was re-dissolved in glacial AcOH (1.2 mL) and purified by preparative HPLC (Phenomenex Jupiter 10 μM Proteo 90 Å LC column, 250×21.2 mm, with flow rate-15 mL/min, gradient of 0-100% acetonitrile in 25 mM aqueous ammonium acetate over 30 minutes) to afford 2.7 mg of E-215 as a white solid. ESI-MS found 1149.8, C₂₁₃H₃₂₅SN₄₇O₆₄ (M+4H)⁴⁺ requires 1149.3.

### Example 216: Compound 226

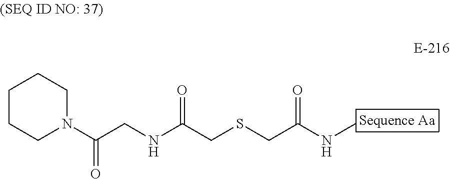 [SMILES: CNC(=O)CSCC(=O)NCC(=O)N1CCCCC1]

Peptide E-216 was prepared from 50 mg of resin R-1 (estimated loading 0.125 mmol/g) using I-478 and GP4 to afford 4.2 mg of E-216 as a white solid. ESI-MS found 1153.4, C₂₁₄H₃₂₇SN₄₇O₆₄ (M+4H)⁴⁺ requires 1152.8.

### Example 217: Compound 219

### The Synthesis of Example E-217 is Depicted in Scheme 168

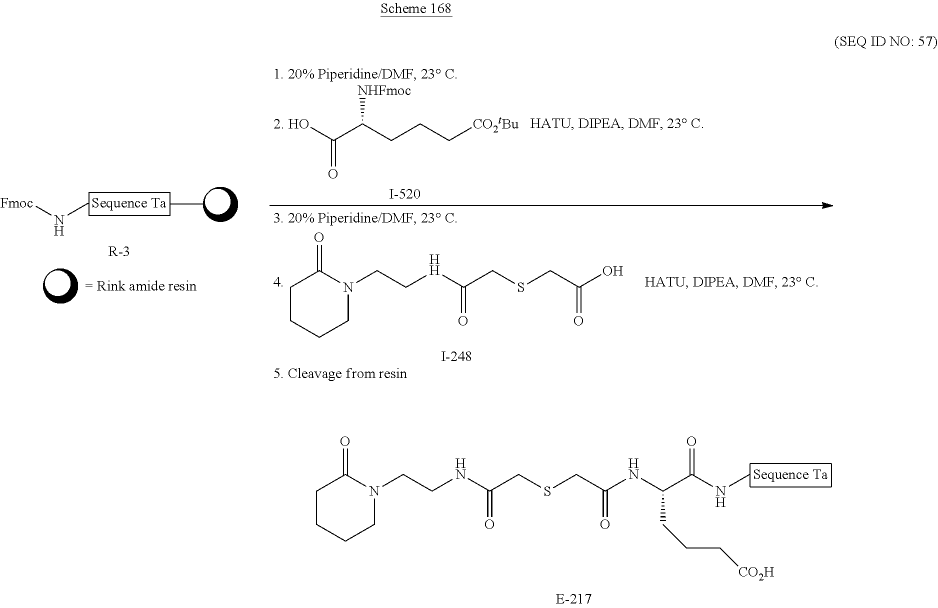

To Rink amide resin R-3 (0.17 mmol/g, 62.5 mg, 0.011 mmol) in a 3 mL polypropylene tube with an end-cap was added 20% v/v piperidine/DMF (2 mL). The tube was capped, agitated at ambient temperature for 20 minutes, and then drained. The resin was washed with DMF (5×3 mL). A solution of carboxylic acid I-520 (28 mg, 6.0 equiv.) in DMF (1.2 mL) was added, followed by DIPEA (28 μL, 15.0 equiv.) and HATU (36.3 mg, 9.0 equiv.). The reaction mixture was agitated at ambient temperature for 4 hours, then drained and the resin was washed with DMF (5×3 mL) and DCM (5×3 mL).

20% v/v piperidine/DMF (2 mL) was added to the resin. The tube was capped, agitated at ambient temperature for 20 minutes, and then drained. The resin was washed with DMF (5×3 mL). A solution of carboxylic acid I-248 (17.5 mg, 6.0 equiv.) in DMF (1.2 mL) was added, followed by DIPEA (28 μL, 15.0 equiv.) and HATU (36.3 mg, 9.0 equiv.). The reaction mixture was agitated at ambient temperature for 5 hours, then drained and the resin was washed with DMF (5×3 mL) and DCM (5×3 mL). The peptide was cleaved from the resin using 1 mL of cleavage reagent (88:2:5:5 v/v/v/v TFA/TIS/PhOH/H₂O) for 1 hour. The resin was filtered and washed with TFA (2×0.5 mL). The combined filtrate and washes were concentrated under reduced pressure to afford a residue, which was triturated with Et₂O (2 mL) to precipitate the peptide. The peptide was re-dissolved in glacial AcOH (1.2 mL) and purified by preparative HPLC (Phenomenex Jupiter 10 μM Proteo 90 Å LC column, 250×21.2 mm, with flow rate-15 mL/min, gradient of 0-100% acetonitrile in 25 mM aqueous ammonium acetate over 30 minutes) to afford 2.5 mg of E-217 as a white solid. ESI-MS found 1156.8, C₂₁₅H₃₂₉SN₄₇O₆₄ (M+4H)⁴⁺ requires 1156.3.

### Example 218: Compound 223

### The Synthesis of Example E-218 is Depicted in Scheme 169

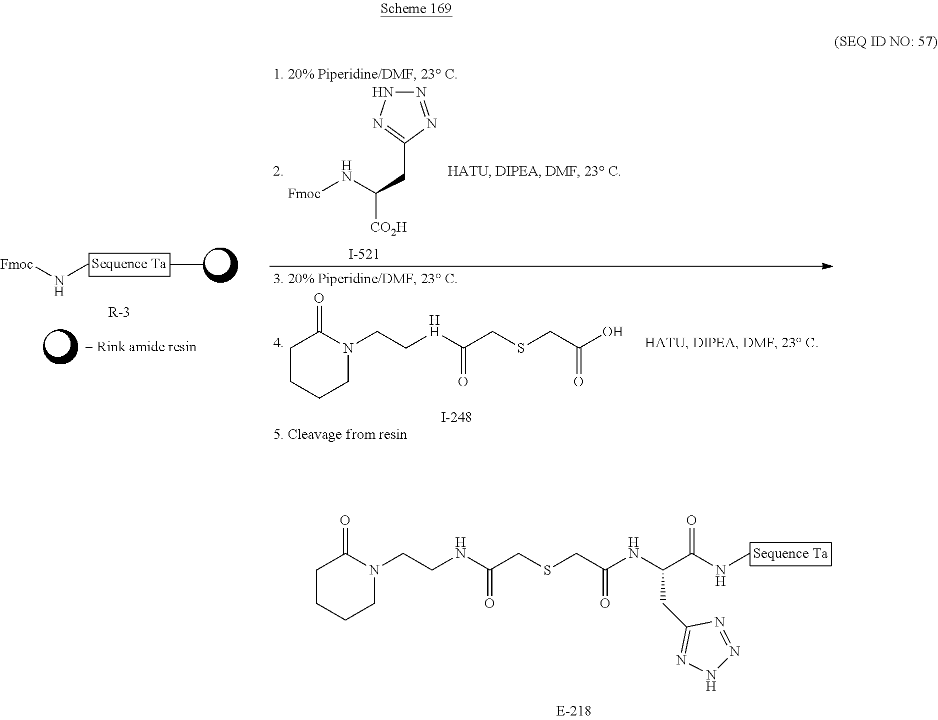

To Rink amide resin R-3 (0.17 mmol/g, 61.4 mg, 0.01 mmol) in a 3 mL polypropylene tube with an end-cap was added 20% v/v piperidine/DMF (2 mL). The tube was capped, agitated at ambient temperature for 20 minutes, and then drained. The resin was washed with DMF (5×3 mL). A solution of carboxylic acid I-521 (23.8 mg, 6.0 equiv.) in DMF (1.2 mL) was added, followed by DIPEA (27 μL, 15.0 equiv.) and HATU (35.6 mg, 9.0 equiv.). The reaction mixture was agitated at ambient temperature for 4 hours, then drained and the resin was washed with DMF (5×3 mL) and DCM (5×3 mL). 20% v/v piperidine/DMF (2 mL) was added to the resin. The tube was capped, agitated at ambient temperature for 20 minutes, and then drained. The resin was washed with DMF (5×3 mL). A solution of carboxylic acid I-248 (17.2 mg, 6.0 equiv.) in DMF (1.2 mL) was added, followed by DIPEA (27 μL, 15.0 equiv.) and HATU (35.6 mg, 9.0 equiv.). The reaction mixture was agitated at ambient temperature for 5 hours, then drained and the resin was washed with DMF (5×3 mL) and DCM (5×3 mL). The peptide was cleaved from the resin using 1 mL of cleavage reagent (88:2:5:5 v/v/v/v TFA/TIS/PhOH/H₂O) for 1 hour. The resin was filtered and washed with TFA (2×0.5 mL). The combined filtrate and washes were concentrated under reduced pressure to afford a residue, which was triturated with Et₂O (2 mL) to precipitate the peptide. The peptide was re-dissolved in glacial AcOH (1.2 mL) and purified by preparative HPLC (Phenomenex Jupiter 10 μM Proteo 90 Å LC column, 250×21.2 mm, with flow rate-15 mL/min, gradient of 0-100% acetonitrile in 25 mM aqueous ammonium acetate over 30 minutes) to afford 1.7 mg of E-218 as a white solid. ESI-MS found 1155.8, C₂₁₃H₃₂₅SN₅₁O₆₂ (M+4H)⁴⁺ requires 1155.3.

### Example 219: Compound 218

### The Synthesis of Example E-219 is Depicted in Scheme 170

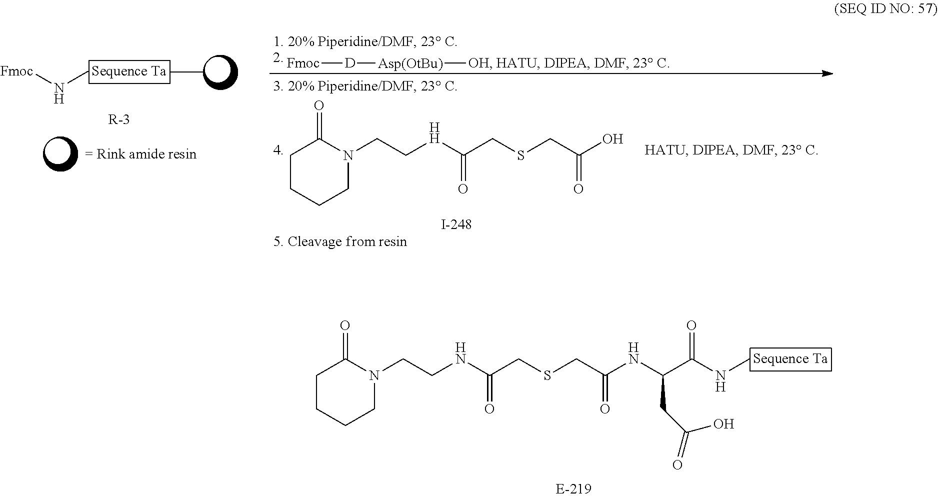

### Scheme 170

Fmoc cleavage from Rink amide resin R-3 (0.17 mmol/g, 66.7 mg, 0.011 mmol) was performed as described in example 218. A solution of Fmoc-D-Asp(O^{t}Bu)-OH (28 mg 6.0 equiv.) in DMF (1.2 mL) was added, followed by DIPEA (30 μL, 15.0 equiv.) and HATU (38.8 mg, 9.0 equiv.). The reaction mixture was agitated at ambient temperature for 4 hours, then drained and the resin was washed with DMF (5×3 mL) and DCM (5×3 mL). After further Fmoc cleavage, A solution of carboxylic acid I-248 (18.7 mg, 6.0 equiv.) in DMF (1.2 mL) was added, followed by DIPEA (28 μL, 15.0 equiv.) and HATU (38.8 mg, 9.0 equiv.). The reaction mixture was agitated at ambient temperature for 5 hours, then drained and the resin was washed with DMF (5×3 mL) and DCM (5×3 mL). The peptide was cleaved from the resin and purified as described in example 218 to afford 4.3 mg of E-219 as a white solid. ESI-MS found 1149.8, C₂₁₃H₃₂₅SN₄₇O₆₄ (M+4H)⁴⁺ requires 1149.3.

### Example 220: Compound 221

### The Synthesis of Example E-220 is Depicted in Scheme 171

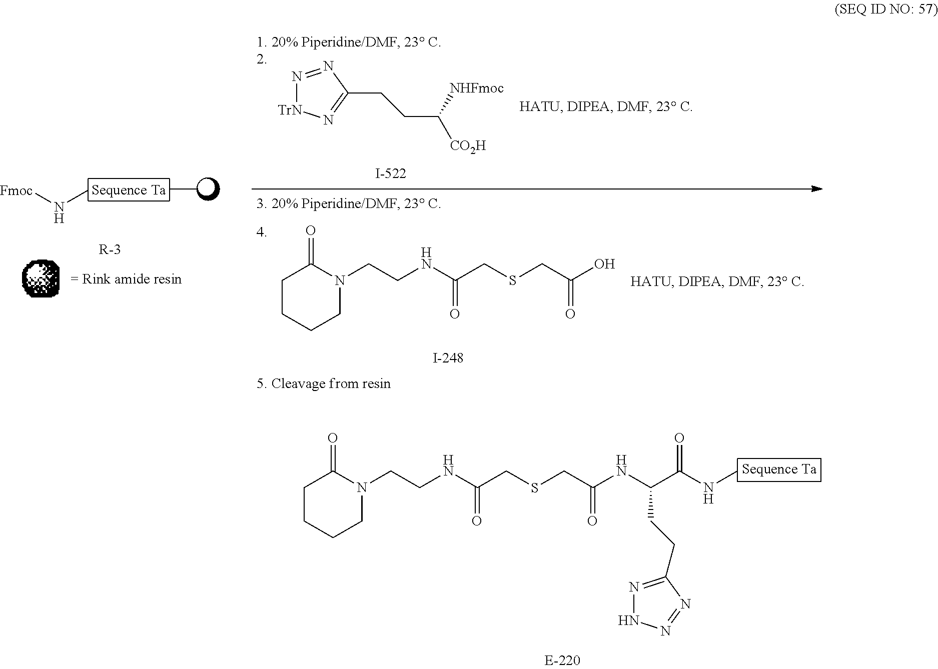

### Scheme 171

Fmoc cleavage from Rink amide resin R-3 (0.17 mmol/g, 65.5 mg, 0.011 mmol) was performed as described in example 218. A solution of carboxylic acid I-522 (42.5 mg 6.0 equiv.) in DMF (1.2 mL) was added, followed by DIPEA (29 μL, 15.0 equiv.) and HATU (38 mg, 9.0 equiv.). The reaction mixture was agitated at ambient temperature for 4 hours, then drained and the resin was washed with DMF (5×3 mL) and DCM (5×3 mL). After further Fmoc cleavage, a solution of carboxylic acid I-248 (18.3 mg, 6.0 equiv.) in DMF (1.2 mL) was added, followed by DIPEA (29 μL, 15.0 equiv.) and HATU (38 mg, 9.0 equiv.). The reaction mixture was agitated at ambient temperature for 5 hours, then drained and the resin was washed with DMF (5×3 mL) and DCM (5×3 mL). The peptide was cleaved from the resin and purified as described in example 218 to afford 2.0 mg of E-220 as a white solid. ESI-MS found 1159.3, C₂₁₄H₃₂₇SN₅₁O₆₂ (M+4H)⁴⁺ requires 1158.8.

### Example 221: Compound 222

### The Synthesis of Example E-221 is Depicted in Scheme 172

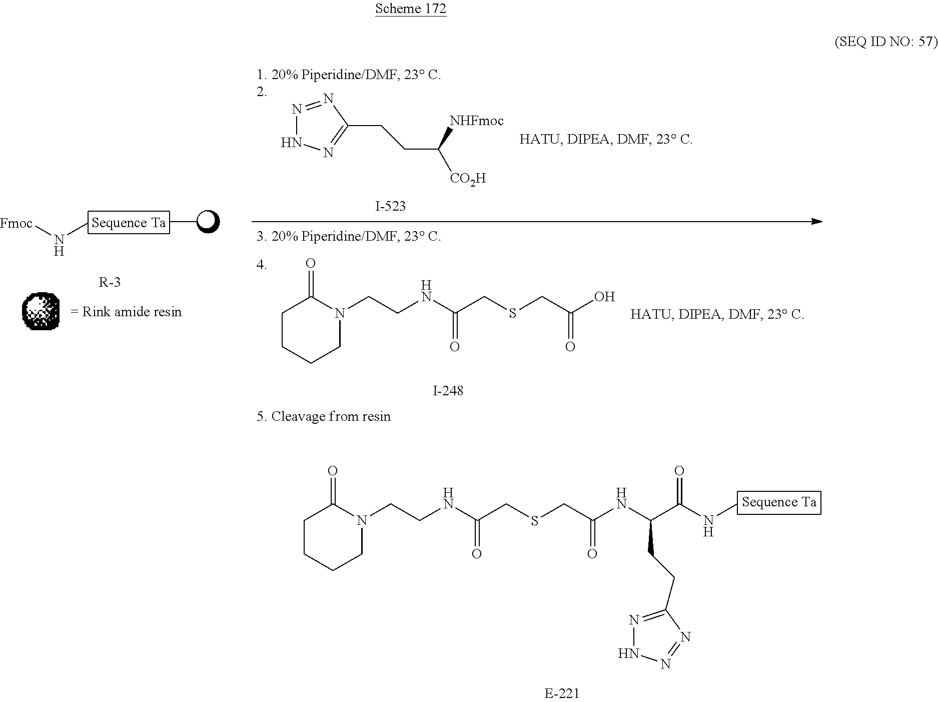

Fmoc cleavage from Rink amide resin R-3 (0.17 mmol/g, 64.8 mg, 0.011 mmol) was performed as described in example 218. A solution of carboxylic acid I-523 (26 mg, 6.0 equiv.) in DMF (1.2 mL) was added, followed by DIPEA (29 μL, 15.0 equiv.) and HATU (37.6 mg, 9.0 equiv.). The reaction mixture was agitated at ambient temperature for 4 hours, then drained and the resin was washed with DMF (5×3 mL) and DCM (5×3 mL). After further Fmoc cleavage, a solution of carboxylic acid I-248 (18.1 mg, 6.0 equiv.) in DMF (1.2 mL) was added, followed by DIPEA (29 μL, 15.0 equiv.) and HATU (37.6 mg 9.0 equiv.). The reaction mixture was agitated at ambient temperature for 5 hours, then drained and the resin was washed with DMF (5×3 mL) and DCM (5×3 mL). The peptide was cleaved from the resin and purified as described in example 218 to afford 1.4 mg of E-221 as a white solid. ESI-MS found 1159.4, C₂₁₄H₃₂₇SN₅₁O₆₂ (M+4H)⁴⁺ requires 1158.8.

### Example 222: Compound 224

### The Synthesis of Example E-222 is Depicted in Scheme 173

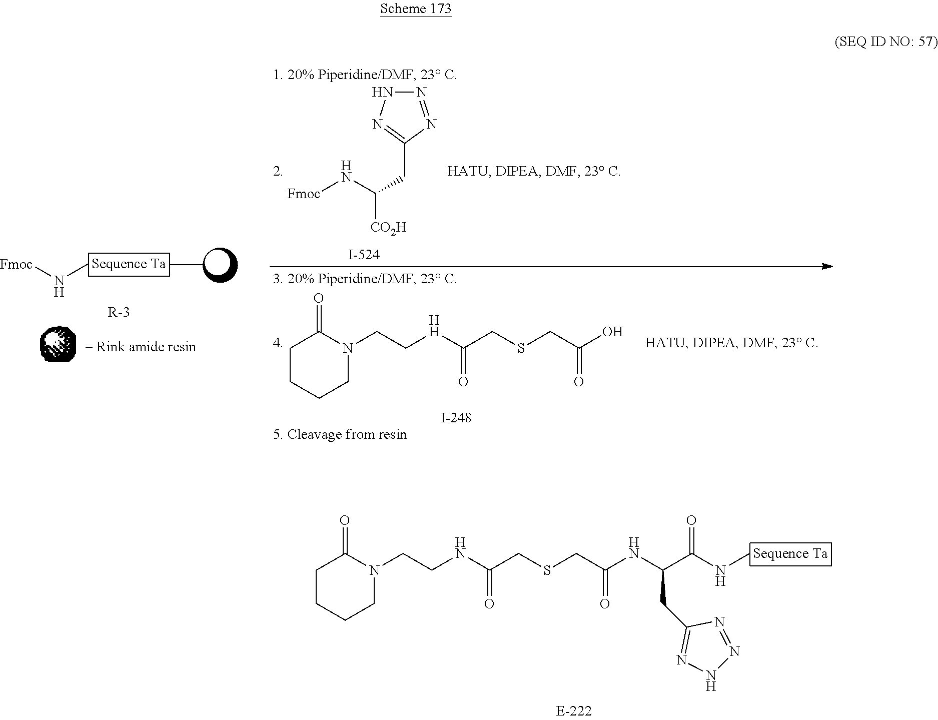

Fmoc cleavage from Rink amide resin R-3 (0.17 mmol/g, 62.8 mg, 0.011 mmol) was performed as described in example 218. A solution of carboxylic acid I-524 (24.3 mg, 6.0 equiv.) in DMF (1.2 mL) was added, followed by DIPEA (29 μL, 15.0 equiv.) and HATU (36.6 mg, 9.0 equiv.). The reaction mixture was agitated at ambient temperature for 4 hours, then drained and the resin was washed with DMF (5×3 mL) and DCM (5×3 mL). After further Fmoc cleavage, a solution of carboxylic acid I-248 (17.6 mg, 6.0 equiv.) in DMF (1.2 mL) was added, followed by DIPEA (29 μL, 15.0 equiv.) and HATU (36.6 mg, 9.0 equiv.). The reaction mixture was agitated at ambient temperature for 5 hours, then drained and the resin was washed with DMF (5×3 mL) and DCM (5×3 mL). The peptide was cleaved from the resin and purified as described in example 218 to afford 2.5 mg of E-222 as a white solid. ESI-MS found 1155.9, C₂₁₃H₃₂₅SN₅₁O₆₂ (M+4H)⁴⁺ requires 1155.3.

### Example 228: Compound 232

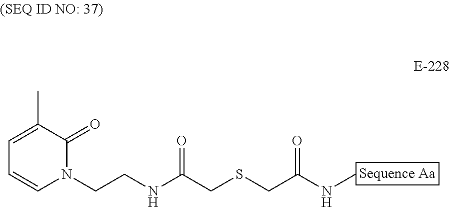 [SMILES: CNC(=O)CSCC(=O)NCCn1cccc(C)c1=O]

Peptide E-228 was prepared from 44.5 mg of resin R-1 (estimated loading 0.125 mmol/g) using I-486 and GP4 to afford 4.8 mg of E-228 as a white solid. ESI-MS found 1155.8, C₂₁₅H₃₂₅SN₄₇O₆₄ (M+4H)⁴⁺ requires 1155.3.

### Example 229: Compound 233

### The Synthesis of Example E-229 is Depicted in Scheme 179

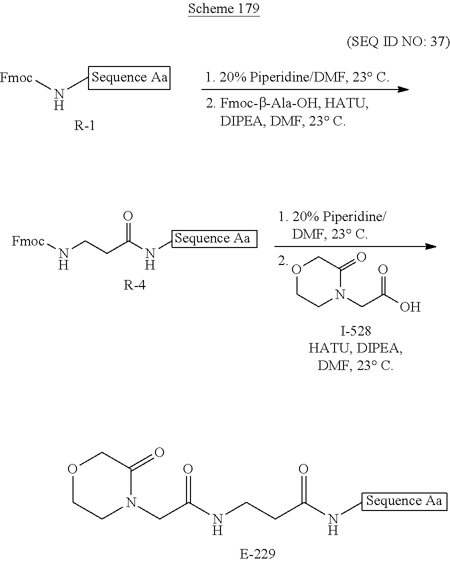

Fmoc cleavage from Rink amide resin R-1 (estimated loading 0.125 mmol/g, 121.6 mg 0.015 mmol) was performed as described in example 218. A solution of Fmoc-β-Ala-OH (28.4 mg, 6.0 equiv.) in DMF (3 mL) was added, followed by DIPEA (40 μL, 15.0 equiv.) and HATU (52 mg, 9.0 equiv.). The reaction mixture was agitated at ambient temperature for 2 hours, then drained and the resin was washed with DMF (5×3 mL) and DCM (5×3 mL) and dried in vacuo to afford resin R-4.

Resin R-4 (49.3 mg, estimated loading 0.125 mmol/g, 6.2 μmol) was subjected to Fmoc cleavage as described above, and a solution of carboxylic acid I-528 (6 mg, 6.0 equiv.) in DMF (1.2 mL) was added, followed by DIPEA (16 μL, 15.0 equiv.) and HATU (21 mg, 9.0 equiv.). The reaction mixture was agitated at ambient temperature for 16 hours, then drained and the resin was washed with DMF (5×3 mL) and DCM (5×3 mL) and dried in vacuo. The peptide was cleaved from the resin and purified as described in example 218 to afford 5.6 mg of Peptide E-229 as a white solid. ESI-MS found 1142.3, C₂₁₂H₃₂₃N₄₇O₆₅ (M+4H)⁴⁺ requires 1141.8.

### Example 230: Compound 234

### The Synthesis of Example E-230 is Depicted in Scheme 180

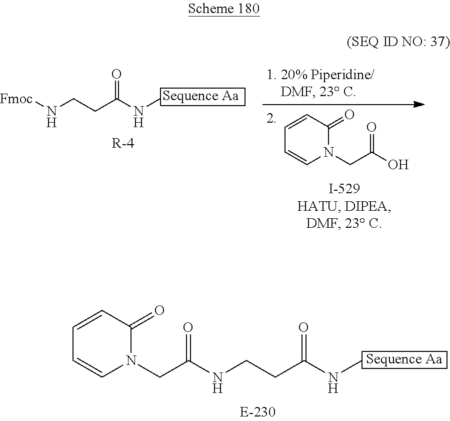

Resin R-4 (52.2 mg, estimated loading 0.125 mmol/g, 6.5 μmol) was subjected to Fmoc cleavage as described in example 218, and a solution of carboxylic acid I-529 (6 mg, 6.0 equiv.) in DMF (1.2 mL) was added, followed by DIPEA (17 μL, 15.0 equiv.) and HATU (22 mg, 9.0 equiv.). The reaction mixture was agitated at ambient temperature for 16 hours, then drained and the resin was washed with DMF (5×3 mL) and DCM (5×3 mL) and dried in vacuo. The peptide was cleaved from the resin and purified as described in example 218 to afford 4.3 mg of E-230 as a white solid. ESI-MS found 1140.8, C₂₁₃H₃₂₁N₄₇O₆₄ (M+4H)⁴⁺ requires 1140.3.

### Example 234: Compound 238

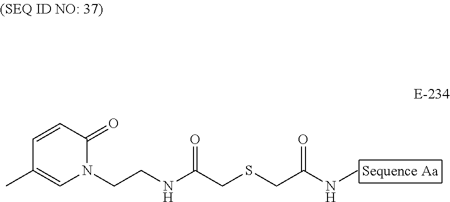

Peptide E-234 was prepared from 46.2 mg of resin R-1 (estimated loading 0.125 mmol/g) using I-498 and GP4 to afford 6.1 mg of E-234 as a white solid. ESI-MS found 1155.8, C₂₁₅H₃₂₅SN₄₇O₆₄ (M+4H)⁴⁺ requires 1155.3.

### Example 235: Compound 239

### The Synthesis of Example E-235 is Depicted in Scheme 184

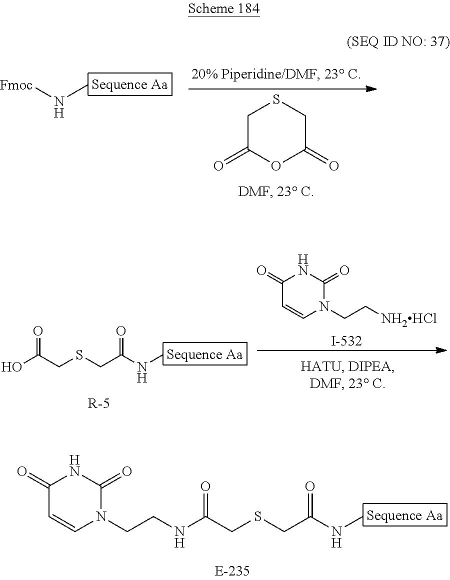

Fmoc deprotection of resin R-1(111.9 mg, estimated loading 0.125 mmol/g, 0.014 mmol) was carried out as described in example 218, then a solution of thiodiglycolic anhydride (14.8 mg, 8.0 equiv.) in DMF (1.2 mL) was added and the reaction mixture was agitated for 6 hours at ambient temperature. The solution was drained and the resin was washed with DMF (5×) and DCM (5×) and dried in vacuo to afford resin R-5.

To resin R-5 (40.5 mg, estimated loading 0.125 mmol/g, 5.1 μmol) was added a solution of amine hydrochloride I-532 (7.8 mg, 8.0 equiv.) and DIPEA (18 μL, 20.0 equiv.) in DMF (1.2 mL), followed by HATU (15.4 mg, 8.0 equiv.). The reaction mixture was agitated at ambient temperature for 5 hours, then the solution was drained and the resin was washed with DMF (5×) and DCM (5×) and dried in vacuo. The peptide was cleaved from resin and purified as described in example 218 to afford 1.7 mg of peptide E-235 as a white solid. ESI-MS found 1156.0, C₂₁₃H₃₂₂SN₄₈O₆₅ (M+4H)⁴⁺ requires 1156.1.

### Example 236: Compound 240

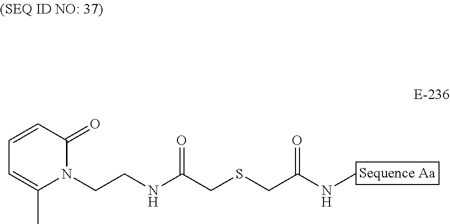 [SMILES: CNC(=O)CSCC(=O)NCCn1c(C)cccc1=O]

Peptide E-236 was prepared from 42.2 mg of resin R-1 (estimated loading 0.125 mmol/g) using I-490 and GP4 to afford 2.7 mg of E-236 as a white solid. ESI-MS found 1155.3, C₂₁₅H₃₂₅SN₄₇O₆₄ (M+4H)⁴⁺ requires 1155.3.

### Example 237: Compound 241

### The Synthesis of Example E-237 is Depicted in Scheme 185

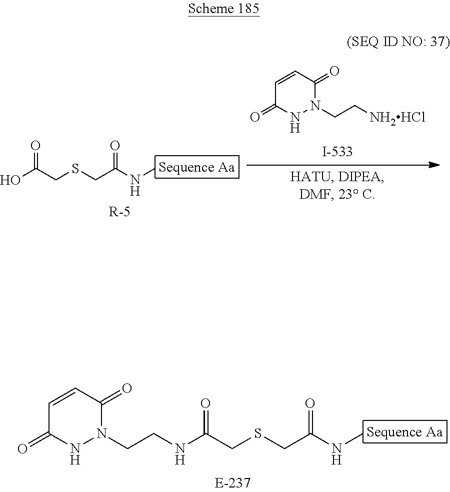

To resin R-5 (41.3 mg, estimated loading 0.125 mmol/g, 5.2 μmol) was added a solution of amine hydrochloride I-533 (7.9 mg, 8.0 equiv.) and DIPEA (18 μL, 20.0 equiv.) in DMF (1.2 mL), followed by HATU (15.7 mg, 8.0 equiv.). The reaction mixture was agitated at ambient temperature for 3.5 hours, then the solution was drained and the resin was washed with DMF (5×) and DCM (5×) and dried in vacuo. The peptide was cleaved from resin and purified as described in example 218 to afford 2.3 mg of peptide E-237 as a white solid. ESI-MS found 1156.0, C₂₁₃H₃₂₂SN₄₈O₆₅ (M+4H)⁴⁺ requires 1156.1.

### Example 239: Compound 215

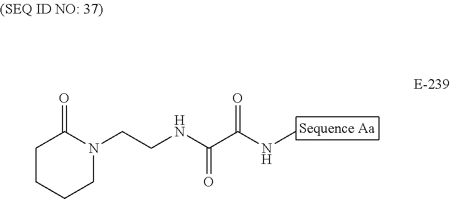 [SMILES: CNC(=O)C(=O)NCCN1CCCCC1=O]

Peptide E-239 was prepared from 49.5 mg of resin (estimated loading 0.125 mmol/g) using I-505 and GP4 to afford 5.3 mg of E-239 as a white solid. ESI-MS found 1137.7, C₂₁₂H₃₂₃N₄₇O₆₄ (M+4H)⁴⁺ requires 1137.8.

### Example 240: Compound 243

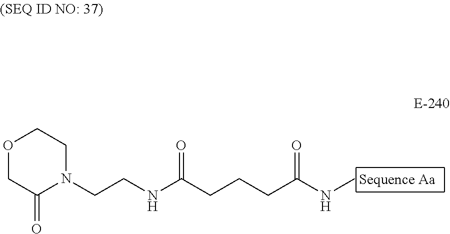 [SMILES: CNC(=O)CCCC(=O)NCCN1CCOCC1=O]

Peptide E-240 was prepared from 47.1 mg of resin (estimated loading 0.125 mmol/g) using I-507 and GP4 to afford 5.0 mg of E-240 as a white solid. ESI-MS found 1148.7, C₂₁₄H₃₂₇N₄₇O₆₅ (M+4H)⁴⁺ requires 1148.8.

### Example 241: Compound 244

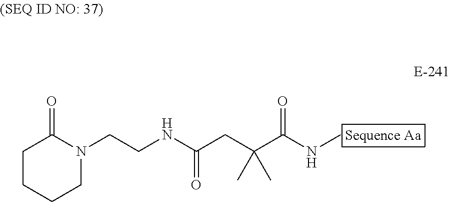 [SMILES: CNC(=O)C(C)(C)CC(=O)NCCN1CCCCC1=O]

Peptide E-241 was prepared from 53.3 mg of resin (estimated loading 0.125 mmol/g) using I-508 and GP4 to afford 3.7 mg of E-241 as a white solid. ESI-MS found 1152.3, C₂₁₅H₃₂₉N₄₇O₆₅ (M+4H)⁴⁺ requires 1152.3.

### Example 244: Compound 247

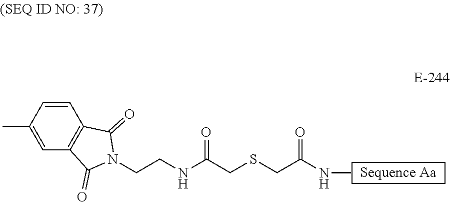

Peptide E-244 was prepared from 47.8 mg of resin (estimated loading 0.125 mmol/g) using I-122 and GP5 to afford 7.0 mg of E-244 as a white solid. ESI-MS found 1168.2, C₂₁₈H₃₂₅SN₄₇O₆₅ (M+4H)⁴⁺ requires 1168.3

### Example 245: Compound 248

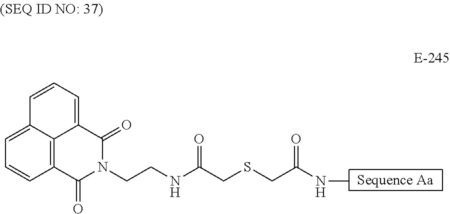 [SMILES: CNC(=O)CSCC(=O)NCCN1C(=O)c2cccc3cccc(c23)C1=O]

Peptide E-245 was prepared from 44.3 mg of resin (estimated loading 0.125 mmol/g) using I-126 and GP5 to afford 3.3 mg of E-245 as a white solid. ESI-MS found 1177.3, C₂₂₁H₃₂₅SN₄₇O₆₅ (M+4H)⁴⁺ requires 1177.3.

### Example 246: Compound 249

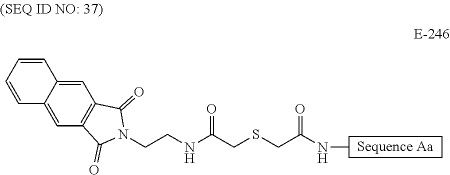 [SMILES: CNC(=O)CSCC(=O)NCCN1C(=O)c2cc3ccccc3cc2C1=O]

Peptide E-246 was prepared from 21.2 mg of resin (estimated loading 0.125 mmol/g) using I-137 and GP5 to afford 1.5 mg of E-246 as a white solid. ESI-MS found 1177.3, C₂₂₁H₃₂₅SN₄₇O₆₅ (M+4H)⁴⁺ requires 1177.3.

### Example 251: Compound 220

### The Synthesis of Example E-251 is Depicted in Scheme 190

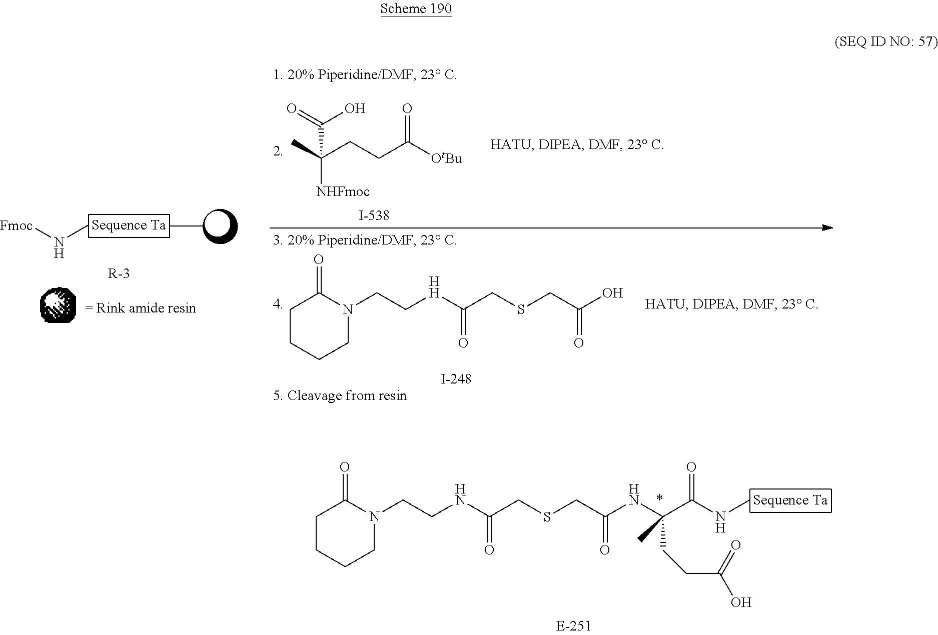

Fmoc cleavage from Rink amide resin R-3 (0.18 mmol/g, 51.5 mg, 0.009 mmol) was performed as described in example 218. A solution of carboxylic acid I-538 (24.4 mg, 6.0 equiv., *absolute configuration not established) in 20% Collidine/DMF (1.5 mL) was added, followed by HATU (19.4 mg, 5.5 equiv.). The reaction mixture was agitated at ambient temperature for 16 hours, then drained and the resin was washed with DMF (5×3 mL) and DCM (5×3 mL). After further Fmoc cleavage, a solution of carboxylic acid I-248 (15.3 mg, 6.0 equiv.) in 20% Collidine/DMF (1.5 mL) was added, followed by HATU (19.4 mg, 5.5 equiv.). The reaction mixture was agitated at ambient temperature for 16 hours, then drained and the resin was washed with DMF (5×3 mL) and DCM (5×3 mL). The peptide was cleaved from the resin and purified as described in example 218 to afford 3.6 mg of E-251 as a white solid. ESI-MS found 1156.8, C₂₁₅H₃₂₉SN₄₇O₆₄ (M+4H)⁴⁺ requires 1156.3.

### Example 252: Compound 254

### The Synthesis of Example E-252 is Depicted in Scheme 191

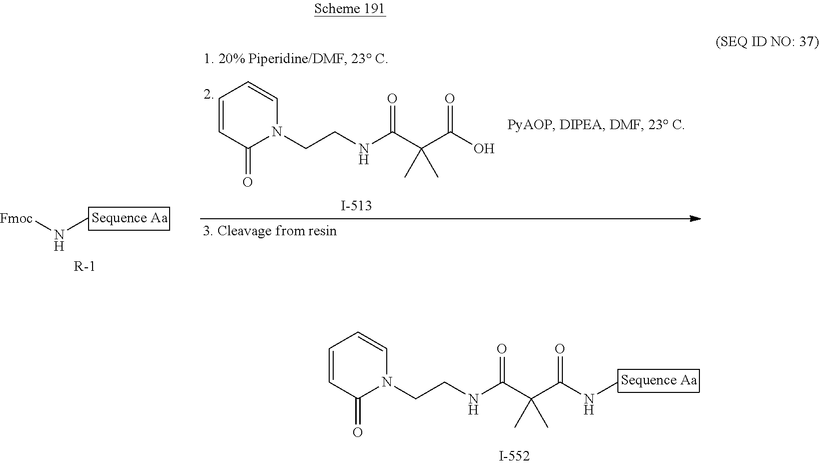

Fmoc cleavage from resin R-1 (estimated loading 0.125 mmol/g, 42 mg, 5.3 μmol) was performed as described in example 218. A solution of carboxylic acid I-513 (8 mg, 6.0 equiv.) in DMF (1.3 mL) was then added to the resin, followed by DIPEA (14 μL, 15.0 equiv.) and PyAOP (19.2 mg, 7.0 equiv.). The reaction mixture was agitated at ambient temperature for 20 hours and then the resin was drained and washed with DMF (5×), DCM (5×) and dried in vacuo. Cleavage of the peptide from resin and purification was performed as described for example 218 to afford 4.0 mg of E-252 as a white solid. ESI-MS found 1147.8, C₂₁₅H₃₂₅N₄₇O₆₄ (M+4H)⁴⁺ requires 1147.3.

### Example 255: Compound 257

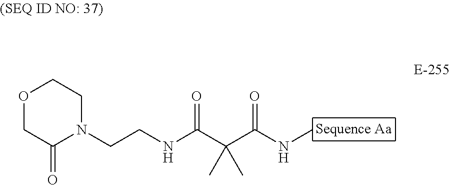 [SMILES: CNC(=O)C(C)(C)C(=O)NCCN1CCOCC1=O]

Peptide E-255 was prepared from 49.7 mg of resin (estimated loading 0.125 mmol/g) using I-510 as described for example 252 (coupling time: 5 hours) to afford 5.7 mg of E-255 as a white solid. ESI-MS found 1149.4, C₂₁₄H₃₂₇N₄₇O₆₅ (M+4H)⁴⁺ requires 1148.8.

### Example 258: Compound 260

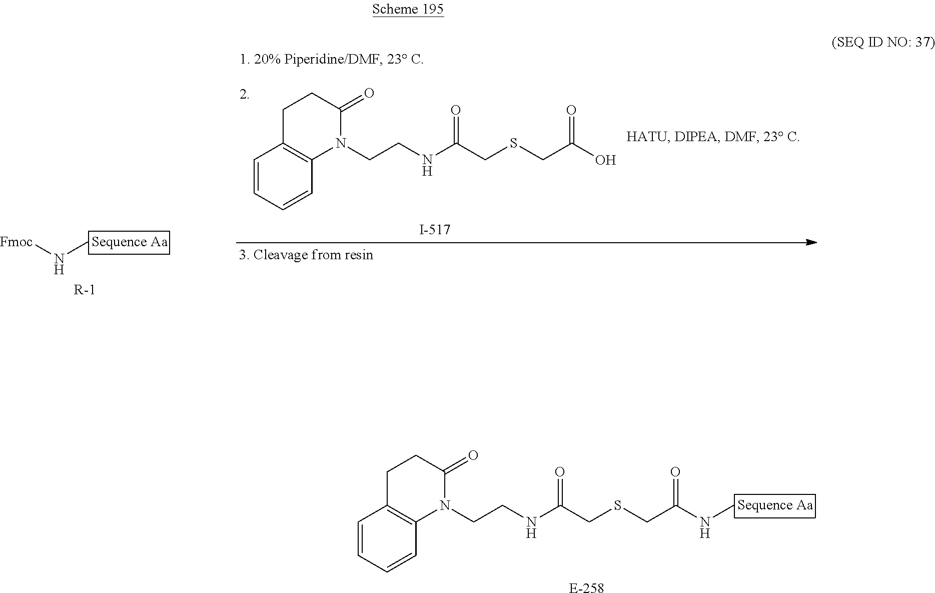

Fmoc cleavage from resin R-1 (estimated loading 0.125 mmol/g, 46.2 mg, 5.8 μmol) was performed as described in example 218. A solution of carboxylic acid I-517 (11.2 mg, 6.0 equiv.) in DMF (1.3 mL) was then added to the resin, followed by DIPEA (15 μL, 15.0 equiv.) and HATU (12.1 mg, 5.5 equiv.). The reaction mixture was agitated at ambient temperature for 2 hours and then the resin was drained and washed with DMF (5×), DCM (5×) and dried in vacuo. Cleavage of the peptide from resin and purification was performed as described for example 218 to afford 3.9 mg of E-258 as a white solid. ESI-MS found 1165.4, C₂₁₈H₃₂₇SN₄₇O₆₄ (M+4H)⁴⁺ requires 1164.8.

### Example 262: Compound 264

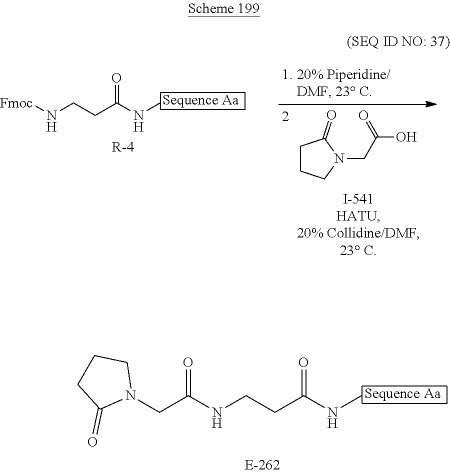

Resin R-4 (39.3 mg, estimated loading 0.125 mmol/g, 4.9 μmol) was subjected to Fmoc cleavage as described in example 218, and a solution of carboxylic acid I-541 (4.2 mg, 6.0 equiv.) in 20% Collidine/DMF (1.3 mL) was added, followed by HATU (10.3 mg, 5.5 equiv.). The reaction mixture was agitated at ambient temperature for 5 hours, then drained and the resin was washed with DMF (5×3 mL) and DCM (5×3 mL) and dried in vacuo. The peptide was cleaved from the resin and purified as described in example 218 to afford 6.5 mg of E-262 as a white solid. ESI-MS found 1138.4, C₂₁₂H₃₂₃N₄₇O₆₄ (M+4H)⁴⁺ requires 1137.8.

### Example 263: Compound 265

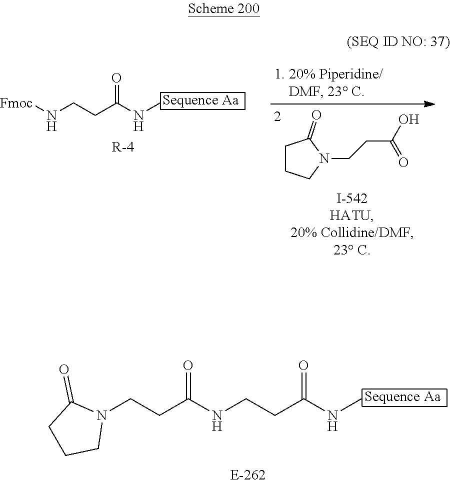

Resin R-4 (40.7 mg, estimated loading 0.125 mmol/g, 5.1 μmol) was subjected to Fmoc cleavage as described in example 218, and a solution of carboxylic acid I-542 (9.2 mg, 11.5 equiv.) in 20% Collidine/DMF (1.3 mL) was added, followed by HATU (10.3 mg, 5.5 equiv.). The reaction mixture was agitated at ambient temperature for 19 hours, then drained and the resin was washed with DMF (5×3 mL) and DCM (5×3 mL) and dried in vacuo. The peptide was cleaved from the resin and purified as described in example 218 to afford 5.0 mg of E-263 as a white solid. ESI-MS found 1141.8, C₂₁₃H₃₂₅N₄₇O₆₄ (M+4H)⁴⁺ requires 1141.3.

### Example 264: Compound 266

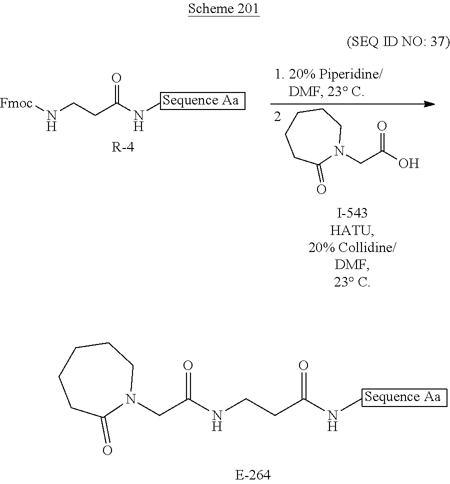

Resin R-4 (43 mg, estimated loading 0.125 mmol/g, 5.4 μmol) was subjected to Fmoc cleavage as described in example 218, and a solution of carboxylic acid I-543 (8.2 mg, 8.9 equiv.) in 20% Collidine/DMF (1.3 mL) was added, followed by HATU (11.2 mg, 5.5 equiv.). The reaction mixture was agitated at ambient temperature for 19 hours, then drained and the resin was washed with DMF (5×3 mL) and DCM (5×3 mL) and dried in vacuo. The peptide was cleaved from the resin and purified as described in example 218 to afford 4.3 mg of E-264 as a white solid. ESI-MS found 1145.3, C₂₁₄H₃₂₇N₄₇O₆₄ (M+4H)⁴⁺ requires 1144.8.

### Example 265: Compound 267

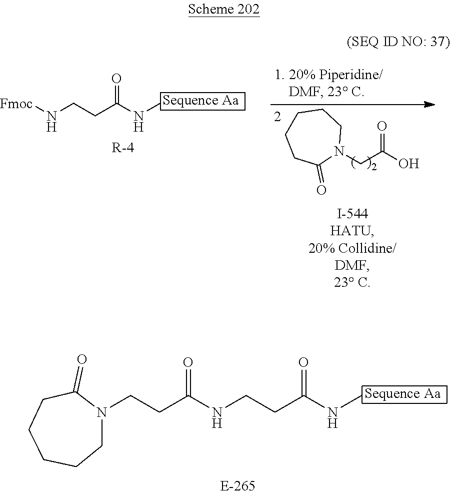

Resin R-4 (36.6 mg, estimated loading 0.125 mmol/g, 4.6 μmol) was subjected to Fmoc cleavage as described in example 218, and a solution of carboxylic acid I-544 (7.3 mg, 8.6 equiv.) in 20% Collidine/DMF (1.3 mL) was added, followed by HATU (9.6 mg, 5.5 equiv.). The reaction mixture was agitated at ambient temperature for 19 hours, then drained and the resin was washed with DMF (5×3 mL) and DCM (5×3 mL) and dried in vacuo. The peptide was cleaved from the resin and purified as described in example 218 to afford 4.4 mg of E-265 as a white solid. ESI-MS found 1148.9, C₂₁₅H₃₂₉N₄₇O₆₄ (M+4H)⁴⁺ requires 1148.3.

### Example 268: Compound 270

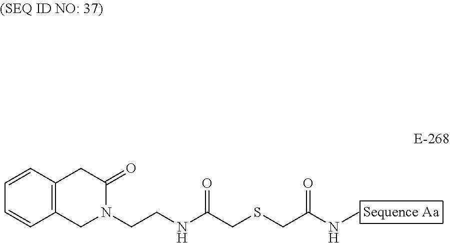 [SMILES: CNC(=O)CSCC(=O)NCCN1Cc2ccccc2CC1=O]

Peptide E-268 was prepared from 55.1 mg of resin R-1 (estimated loading 0.125 mmol/g) using I-518 as described in example 252 to afford 5.7 mg of E-268 as a white solid. ESI-MS found 1165.4, C₂₁₈H₃₂₇SN₄₇O₆₄ (M+4H)⁴⁺ requires 1164.8.

### Example 283: Compound 225

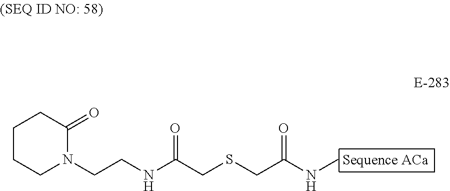 [SMILES: CNC(=O)CSCC(=O)NCCN1CCCCC1=O]

Peptide E-283 was prepared from 102 mg of the corresponding sequence immobilized on Rink amide resin (estimated loading 0.19 mmol/g) using I-248 and GP4 to afford 6.6 mg of E-283 as a white solid. ESI-MS found 1156.8, C₂₁₅H₃₂₉SN₄₇O₆₄ (M+4H)⁴⁺ requires 1156.3.

### Example 284: Compound 285

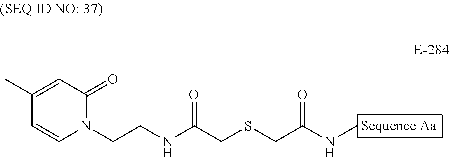

Peptide E-284 was prepared from 50.2 mg of resin R-1 (estimated loading 0.125 mmol/g) using I-494 and GP4 to afford 5.1 mg of E-284 as a white solid. ESI-MS found 1155.8, C₂₁₅H₃₂₅SN₄₇O₆₄ (M+4H)⁴⁺ requires 1155.3.

### Example 288: Compound 289

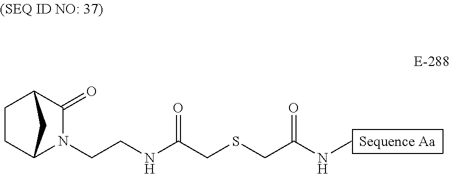 [SMILES: CNC(=O)CSCC(=O)NCCN1C(=O)[C@H]2CC[C@@H]1C2]

Peptide E-288 was prepared from 72.4 mg of resin R-1 (estimated loading 0.125 mmol/g) using I-552 as described in example 254 to afford 7.5 mg of E-288 as a white solid. ESI-MS found 1156.4, C₂₁₅H₃₂₇N₄₇O₆₄S (M+4H)⁴⁺ requires 1155.8.

### Example 289: Compound 290

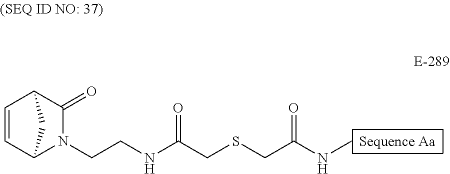 [SMILES: CNC(=O)CSCC(=O)NCCN1C(=O)[C@@H]2C=C[C@H]1C2]

Peptide E-289 was prepared from 65.4 mg of resin R-1 (estimated loading 0.125 mmol/g) using I-557 as described in example 254 to afford 9.3 mg of E-289 as a white solid. ESI-MS found 1155.8, C₂₁₅H₃₂₅N₄₇O₆₄S (M+4H)⁴⁺ requires 1155.3.

### Example 290: Compound 291

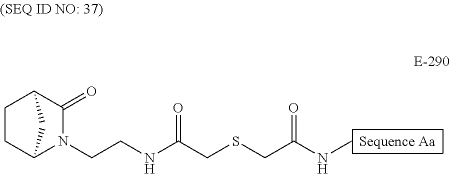 [SMILES: CNC(=O)CSCC(=O)NCCN1C(=O)[C@@H]2CC[C@H]1C2]

Peptide E-290 was prepared from 70.7 mg of resin R-1 (estimated loading 0.125 mmol/g) using I-559 as described in example 254 to afford 4.8 mg of E-290 as a white solid. ESI-MS found 1156.4, C₂₁₅H₃₂₇N₄₇O₆₄S (M+4H)⁴⁺ requires 1155.8.

### Example 291: Compound 292

### The Synthesis of Example E-291 is Depicted in Scheme 208

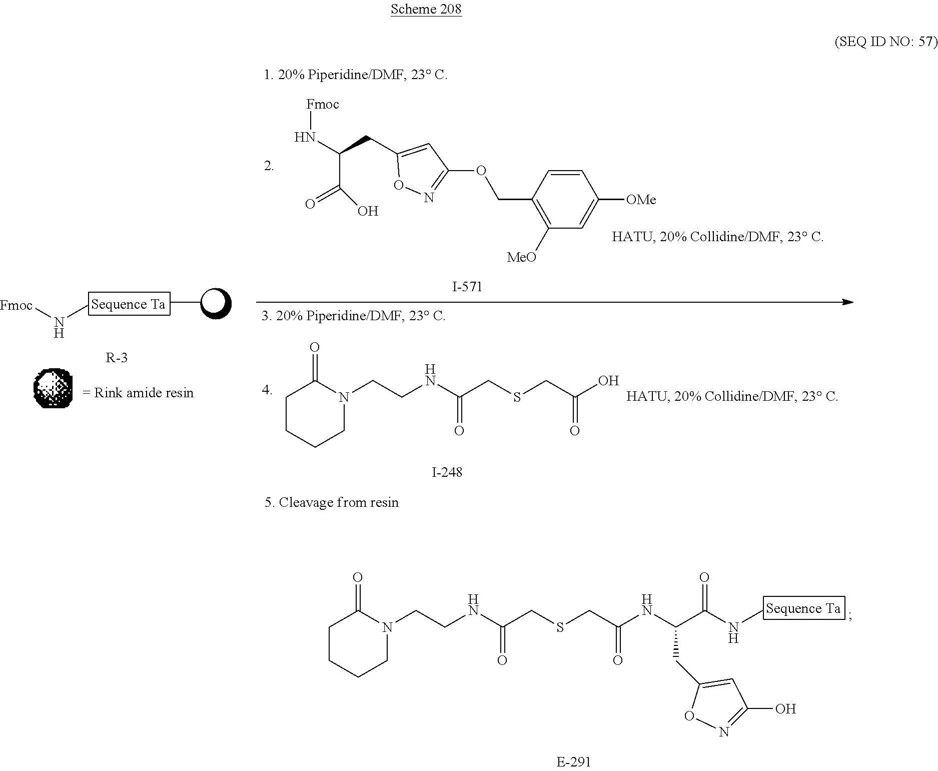

Fmoc cleavage from Rink amide resin R-3 (0.19 mmol/g, 79.1 mg, 0.015 mmol) was performed as described in example 218. A solution of carboxylic acid I-571 (49 mg, 6.0 equiv.) in 20% Collidine/DMF (1.5 mL) was added, followed by HATU (31.4 mg, 5.5 equiv.). The reaction mixture was agitated at ambient temperature for 14.5 hours, then drained and the resin was washed with DMF (5×3 mL) and DCM (5×3 mL). After further Fmoc cleavage, a solution of carboxylic acid I-248 (24.7 mg, 6.0 equiv.) in 20% Collidine/DMF (1.5 mL) was added, followed by HATU (31.4 mg, 5.5 equiv.). The reaction mixture was agitated at ambient temperature for 6.5 hours, then drained and the resin was washed with DMF (5×3 mL) and DCM (5×3 mL). The peptide was cleaved from the resin and purified as described in example 218 to afford 5.3 mg of E-291 as a white solid. ESI-MS found 1159.4, C₂₁₅H₃₂₆SN₄₈O₆₄ (M+4H)₄₊ requires 1159.1.

### E. Biological Assays

### a) HitHunter CAMP Assay

Suspension cells were harvested and resuspended in 1×HBSS (+10 mM HEPES, 625 μM IBMX, 0.2% BSA) according to the following procedure: 5 μL of 2× cell suspension+1× antibody were added to each well, where the optimal cell density was 10,000 cells per well in a low volume 384-well plate. Agonist serial dilutions in a separate 384 well dilution plate in a 22-point series of 2× dilutions of agonist in DMSO were performed as follows. 80 μL of the highest concentration of Agonist/DMSO was added to well No. 1. 40 μL was removed from well No. 1 and added it to well No. 2, followed by gentle mixing. 40 μL was removed from well No. 2 and added it to well No. 3, followed by gentle mixing. This process was repeated until well No. 22. Additional serial dilutions for additional agonists were set up in a similar manner. 50 nL of each 2× agonist serial dilution was added in duplicate to the designated agonist rows of the assay plate using the Labcyte ECHO. Assay plate was incubated for 30 minutes at room temperature. Following agonist incubation, 5 μL of cAMP Antibody Reagent was added to all wells. A stock of CAMP Working Detection Solution in a separate 15 ml polypropylene tube was prepared by mixing 19 parts of cAMP Lysis Buffer, 5 parts of Substrate Reagent 1, 1 part Substrate Reagent 2, and 25 parts of cAMP Solution D. 5 μL of cAMP Working Detection Solution was added to all wells of the assay plate [Note: Do not pipette up and down in the vial to mix or vortex plates]. Plates were spun down briefly. Assay plate was incubated for 1 hour at room temperature in the dark for the immunocompetition reaction to occur. 5 μL of CAMP Solution A was added to all wells of the assay plate [Note: Do not pipette up and down in the vial to mix or vortex plates]. Plates were spun down briefly. Assay plate was incubated O/N at room temperature in the dark. Samples were read on Tecan i-control plate reader using luminescence mode with 300 ms integration time. Data analysis was performed using GraphPad Prism. Results of the HitHunter CAMP assay are presented in Table 3 and Table 3A.

### Note:

In table 3 “+++++” denotes EC₅₀<0.1 nM; “++++” denotes 0.1 nM≤EC₅₀<1 nM; “+++” denotes 1 nM≤EC₅₀<10 nM; “++” denotes 10 nM≤EC₅₀<100 nM; “+” denotes 100 nM≤EC₅₀≤1000 nM; and “−” denotes EC₅₀>1000 nM.
In table 3A, for GLP-1R data: “+++++” denotes EC₅₀<0.1 nM; “++++” denotes 0.1 nM≤EC₅₀<1 nM; “+++” denotes 1 nM≤EC₅₀<10 nM; “++” denotes 10 nM≤EC₅₀<100 nM; “+” denotes 100 nM≤EC₅₀≤1000 nM; and “−” denotes EC₅₀>1000 nM.
In table 3A, for GIP-R data: “+++++” denotes EC₅₀<0.1 nM; “++++” denotes 0.1 nM≤EC₅₀<1 nM; “+++” denotes 1 nM≤EC₅₀<10 nM; “++” denotes 10 nM≤EC₅₀<100 nM or Emax<30%; “+” denotes 100 nM≤EC₅₀≤1000 nM or Emax<20%; and “−” denotes EC₅₀>1000 nM or Emax<10%.

TABLE 3

| Compound | EC₅₀ (Eₘₐₓ) | EC₅₀ (Eₘₐₓ) |
|---|---|---|
| # | GLP-1R/nM | GIP-R/nM |
| 1 | ++++ (97) | +++ (23) |
| 2 | +++++ (99) | +++ (46) |
| 3 | ++++ (96) | — |
| 4 | ++++ (100) | +++ (23) |
| 5 | +++++ (95) | +++ (74) |
| 6 | ++++ (95) | +++ (75) |
| 7 | +++++ (94) | +++ (29) |
| 8 | +++++ (90) | +++ (57) |
| 9 | +++++ (100) | +++ (48) |
| 10 | ++++ (95) | +++ (32) |
| 11 | ++++ (96) | +++ (39) |
| 12 | +++ (91) | ++++ (85) |
| 13 | +++ (69) | +++ (25) |
| 14 | ++++ (81) | ++ (<10) |
| 15 | ++++ (93) | +++ (20) |
| 16 | +++++ (97) | +++ (22) |
| 17 | ++++ (92) | +++ (55) |
| 18 | +++++ (101) | ++++ (80) |
| 19 | +++++ (93) | ++++ (50) |
| 20 | ++++ (91) | +++ (22) |
| 21 | +++++ (95) | +++ (20) |
| 22 | ++++ (90) | ++++ (46) |
| 23 | +++ (83) | +++ (37) |
| 24 | ++++ (92) | +++ (15) |
| 25 | +++ (86) | +++ (43) |
| 26 | ++++ (95) | +++ (64) |
| 27 | ++++ (95) | +++ (45) |
| 28 | ++++ (101) | +++ (57) |
| 29 | +++ (98) | +++ (11) |
| 30 | ++++ (97) | +++ (59) |
| 31 | +++ (102) | ++ (37) |
| 32 | +++++ (103) | +++ (65) |
| 33 | ++++ (100) | ++ (43) |
| 34 | ++++ (107) | +++ (20-38) |
| 35 | ++++ (110) | +++ (20-37) |
| 36 | +++++/++++ (108) | ++++/+++ (51-75) |
| 37 | +++++/++++ (110) | +++ (10-21) |
| 38 | ++++ (97-104) | +++ (18-43) |
| 39 | +++++/++++ | ++++/+++ (40-60) |
| | (102-104) | |
| 40 | ++++ (101-105) | +++ (14-34) |
| 41 | ++++ (100-104) | +++ (75) |
| 42 | +++++/++++ | ++++/+++ (28-46) |
| | (102-109) | |
| 43 | ++++ (93-97) | +++ (60-73) |
| 44 | ++++ (97-99) | +++ (54-67) |
| 45 | ++++ (100) | +++ (42) |
| 46 | +++ (100) | +++ (46) |
| 47 | +++++ (100) | +++ (24) |
| 48 | ++++ (104) | ++++ (65) |
| 49 | ++++ (101) | ++++ (48) |
| 50 | ++++ (103) | +++ (26) |
| 51 | ++++ (98) | +++ (65) |
| 52 | +++++ (101) | +++ (56-61) |
| 53 | +++ (99) | +++ (62) |
| 54 | +++++ (107) | (6 at 1 μM) |
| 55 | ++++ (104) | +++ (47) |
| 56 | ++++ (110) | +++ (60) |
| 57 | ++++ (109) | (7 at 1 μM) |
| 58 | ++++ (108) | +++ (26) |
| 59 | +++++ (108) | +++ (31) |
| 60 | ++++ (98) | +++ (37) |
| 61 | +++ (96) | +++ (56) |
| 62 | +++ (101) | +++ (48) |
| 63 | +++ (91) | +++ (24) |
| 64 | +++++ (97) | +++ (35) |
| 65 | ++++ (96) | +++ (42) |
| 66 | +++++ (113) | +++ (11) |
| 67 | ++++ (100) | +++ (65) |
| 68 | ++++ (109) | ++++ (87) |
| 69 | ++++ (104) | +++ (75) |
| 70 | ++++ (102) | +++ (86) |
| 71 | ++++/+++ (102) | +++ (77-87) |
| 72 | ++++ (105) | ++++ (87) |
| 73 | ++++ (93) | ++ (78) |
| 74 | ++++ (92) | +++ (47) |
| 75 | +++ (99) | +++ (39) |
| 76 | +++ (95) | ++++ (89) |
| 77 | ++++ (90) | ++++ (82) |
| 78 | ++++ (94) | +++ (58) |
| 79 | ++++ (102) | ++++ (77) |
| 80 | ++++ (92) | +++ (73) |
| 81 | ++++ (96) | ++++ (89) |
| 82 | +++ (95) | +++ (82) |
| 83 | ++++ (106) | +++ (73) |
| 84 | ++++(107) | +++ (53) |
| 85 | +++ (103) | ++++ (83) |
| 86 | ++++ (110) | +++ (61) |
| 87 | +++ (89) | +++ (62) |
| 88 | +++ (108) | +++ (102) |
| 89 | +++ (108) | ++++ (81) |
| 90 | +++ (108) | +++ (74) |
| 91 | +++ (98) | +++ (85) |
| 92 | +++ (94-104) | ++++ (85-88) |
| 93 | ++++ (99) | +++ (73) |
| 94 | +++ (89) | +++ (46) |
| 95 | ++ (84) | +++ (20) |
| 96 | +++ (94) | ++++ (90) |
| 97 | +++ (99) | ++++ (93) |
| 98 | ++++ (96) | − (<5 at 1 μM) |
| 99 | +++ (102) | − (11 at 1 μM) |
| 100 | ++ (95) | +++ (100) |
| 101 | ++ (92) | — |
| 102 | +++ (104) | ++ (34) |
| 103 | ++ (95) | +++ (68) |
| 104 | +++ (104-106) | +++ (43-47) |
| 105 | ++ (90) | ++ (<10 at 1 μM) |
| 106 | ++ (95) | ++ (37) |
| 107 | ++ (57) | ++++ (124) |
| 108 | − (18 at 1 μM) | ++ (69) |
| 109 | +++ (101) | ++ (36) |
| 110 | ++ (88) | ++ (33) |
| 111 | ++ (94) | ++ (12 at 1 μM) |
| 112 | ++ (94) | ++ (26 at 1 μM) |
| 113 | ++ (89) | ++ (52) |
| 114 | ++++/+++ (94-103) | +++ (42-63) |
| 115 | +++ (91) | +++ (67) |
| 116 | +++++ (98) | ++ (50) |
| 117 | ++ (92) | ++ (70) |
| 118 | +++++ (95) | +++ (35) |
| 119 | +++++ (97) | +++ (41) |
| 120 | +++ (91) | − (6 at 1 μM) |
| 121 | ++++ (90) | ++ (27) |
| 122 | +++ (91) | − (<1 at 1 μM) |
| 123 | +++ (104) | ++++ (111) |
| 124 | ++ (73) | − (10 at 1 μM) |
| 125 | ++ (94-100) | +++ (54-60) |
| 126 | ++ (84) | ++ (42-51) |
| 127 | +++ (95-107) | +++ (98-102) |
| 128 | +++ (99) | +++/++ (68) |
| 129 | +++/++ (91-95) | ++++/+++ (102-109) |
| 130 | ++++ (114) | +++ (64) |
| 131 | +++ (95) | − (3.2) |
| 132 | +++++ (103-114) | +++ (61-64) |
| 133 | +++ (98) | ++++ (104) |
| 134 | +++++ (103-108) | ++ (30) |
| 135 | +++++/++++ | ++ (15-22) |
| | (106-111) | |
| 136 | ++ (91-94) | ++++ (105) |
| 137 | +++ (103) | ++ (8) |
| 138 | +++ (87) | +++ (81) |
| 139 | ++++ (102) | +++ (79) |
| 140 | +++++ (107) | +++ (63) |
| 141 | +++ (98-100) | ++ (46-50) |
| 142 | +++++ (101-105) | ++ (14.2-17) |
| 143 | ++++ (99-108) | +++ (99-107) |
| 144 | +++ (93-119) | +++++ (127-140) |
| 145 | +++ (100-104) | +++++/++++ (125) |
| 146 | +++ (86-92) | ++ (83-97) |
| 147 | +++ (87-97) | +++++ (114-128) |
| 148 | ++++ (99) | +++ (64-67) |
| 149 | +++ (86-92) | +++++ (113) |
| 150 | +++ (95-113) | +++++/++++ |
| | | (118-130) |
| 151 | +++ (103-107) | ++++ (117-119) |
| 152 | ++ (85) | +++ (103) |
| 153 | +++++ (107) | ++ (75-85) |
| 154 | +++ (80) | +++ (78) |
| 155 | ++++ (102) | +++ (80) |
| 156 | +++ (95-100) | +++++ (116-118) |
| 157 | +++/++ (93-95) | ++++/+++ (103-105) |
| 158 | ++ (31-38) | ++++ (104-107) |
| 159 | ++ (87-91) | +++ (89-92) |
| 160 | +++ (85-95) | ++++ (109-113) |
| 161 | ++ (89-101) | +++/++ (58-107) |
| 162 | ++ (42-46) | ++++/+++ (103-107) |
| 163 | ++ (73) | +++++ (106) |
| 164 | ++ (68) | ++ (99-105) |
| 165 | ++++ (101-106) | +++ (91-98) |
| 166 | +++++ (109) | +++/++ (77-84) |
| 167 | ++++/+++ (94-99) | − (<1 at 1 μM) |
| 168 | +++ (92) | − (6-14 at 1 μM) |
| 169 | +++ (96) | − (6-8 at 1 μM) |
| 170 | +++ (99) | − (5-8 at 1 μM) |
| 171 | +++ (93-98) | − (6.5 at 1 μM) |
| 172 | +++ (98-101) | ++ (63-71) |
| 173 | +++ (90-105) | ++++ (92-111) |
| 174 | ++/+ (17-73) | + (30-66) |
| 175 | ++ (87-94) | ++ (38) |
| 176 | ++ (92) | − (1.8 at 1 μM) |
| 177 | +++ (96) | − (9 at 1 μM) |
| 178 | +++ (97) | + (38) |
| 179 | ++++ (103) | − (2 at 1 μM) |
| 180 | ++ (94) | ++ (84) |
| 181 | ++ (103) | +++ (102) |
| 182 | ++++ (96) | + (12 at 1 μM) |
| 183 | ++ (95) | − (1.4 at 1 μM) |
| 184 | +++ (99) | ++ (43) |
| 185 | ++++ (97) | − (8.6 at 1 μM) |
| 186 | +++ (94) | − (4 at 1 μM) |
| 187 | ++ (89) | ++ (58) |
| 188 | ++ (101) | +++ (107) |
| 189 | +++ (94) | − (4.5 at 1 μM) |
| 190 | +++ (97-119) | ++++/+++ (88-119) |
| 191 | +++ (97) | ++ (68) |
| 192 | ++++ (111) | ++ (63) |
| 193 | +++ (117) | ++ (44) |
| 194 | +++ (98) | − (12 at 1 μM) |
| 195 | +++++++ (93-113) | ++++/+++ (46-87) |
| 196 | ++++/+++ (93-104) | +++/++ (41-86) |

TABLE 3A

| Compound | EC₅₀ (Eₘₐₓ₎ | EC₅₀ (Eₘₐₓ) |
|---|---|---|
| # | GLP-1R/nM | GIP-R/nM |
| 1 | ++++ (97) | ++ (23) |
| 2 | +++++ (99) | +++ (46) |
| 3 | ++++ (96) | — |
| 4 | +++++ (100) | ++ (23) |
| 5 | +++++ (95) | +++ (74) |
| 6 | ++++ (95) | +++ (75) |
| 7 | +++++ (94) | ++ (29) |
| 8 | +++++ (90) | +++ (57) |
| 9 | +++++ (100) | +++ (48) |
| 10 | ++++ (95) | +++ (32) |
| 11 | ++++ (96) | +++ (39) |
| 12 | +++ (91) | ++++ (85) |
| 13 | +++ (69) | ++ (25) |
| 14 | ++++ (81) | — |
| 15 | ++++ (93) | ++ (20) |
| 16 | +++++ (97) | ++ (22) |
| 17 | ++++ (92) | +++ (55) |
| 18 | +++++ (101) | ++++ (80) |
| 19 | +++++ (93) | ++++ (50) |
| 20 | ++++ (91) | ++ (22) |
| 21 | +++++ (95) | ++ (20) |
| 22 | ++++ (90) | ++++ (46) |
| 23 | +++ (83) | +++ (37) |
| 24 | ++++ (92) | + (15) |
| 25 | +++ (86) | +++ (43) |
| 26 | ++++ (95) | +++ (64) |
| 27 | ++++ (95) | +++ (45) |
| 28 | ++++ (101) | +++ (57) |
| 29 | +++ (98) | + (11) |
| 30 | ++++ (97) | +++ (59) |
| 31 | +++ (102) | ++ (37) |
| 32 | +++++ (103) | +++ (65) |
| 33 | ++++ (100) | ++ (43) |
| 34 | ++++ (107) | ++/+++ (20-38) |
| 35 | ++++ (110) | ++/+++ (20-37) |
| 36 | +++++/++++ (108) | ++++/+++ (51-75) |
| 37 | +++++/++++ (110) | +/++ (10-21) |
| 38 | ++++ (97-104) | +/+++ (18-43) |
| 39 | +++++/++++ | ++++/+++ (40-60) |
| | (102-104) | |
| 40 | ++++ (101-105) | +/+++ (14-34) |
| 41 | ++++ (100-104) | +++ (75) |
| 42 | +++++/++++ | ++++/++ (28-46) |
| | (102-109) | |
| 43 | ++++ (93-97) | +++ (60-73) |
| 44 | ++++ (97-99) | +++ (54-67) |
| 45 | ++++ (100) | +++ (42) |
| 46 | +++ (100) | +++ (46) |
| 47 | +++++ (100) | ++ (24) |
| 48 | ++++ (104) | ++++ (65) |
| 49 | ++++ (101) | ++++ (48) |
| 50 | ++++ (103) | ++ (26) |
| 51 | ++++ (98) | +++ (65) |
| 52 | +++++ (101) | +++ (56-61) |
| 53 | +++ (99) | +++ (62) |
| 54 | +++++ (107) | — |
| 55 | ++++ (104) | +++ (47) |
| 56 | ++++ (110) | +++ (60) |
| 57 | ++++ (109) | — |
| 58 | ++++ (108) | ++ (26) |
| 59 | +++++ (108) | +++ (31) |
| 60 | ++++ (98) | +++ (37) |
| 61 | +++ (96) | +++ (56) |
| 62 | +++ (101) | +++ (48) |
| 63 | +++ (91) | ++ (24) |
| 64 | +++++ (97) | +++ (35) |
| 65 | ++++ (96) | +++ (42) |
| 66 | +++++ (113) | + (11) |
| 67 | ++++ (100) | +++ (65) |
| 68 | ++++ (109) | ++++ (87) |
| 69 | ++++ (104) | +++ (75) |
| 70 | ++++ (102) | +++ (86) |
| 71 | ++++/+++ (102) | +++ (77-87) |
| 72 | ++++ (105) | ++++ (87) |
| 73 | ++++ (93) | ++ (78) |
| 74 | ++++ (92) | +++ (47) |
| 75 | ++++ (99) | +++ (39) |
| 76 | +++ (95) | ++++ (89) |
| 77 | ++++ (90) | ++++ (82) |
| 78 | ++++ (94) | +++ (58) |
| 79 | ++++ (102) | ++++ (77) |
| 80 | ++++ (92) | +++ (73) |
| 81 | ++++ (96) | ++++ (89) |
| 82 | +++ (95) | +++ (82) |
| 83 | ++++ (106) | +++ (73) |
| 84 | ++++(107) | +++ (53) |
| 85 | +++ (103) | ++++ (83) |
| 86 | ++++ (110) | +++ (61) |
| 87 | +++ (89) | +++ (62) |
| 88 | +++ (108) | +++ (102) |
| 89 | +++ (108) | ++++ (81) |
| 90 | +++ (108) | +++ (74) |
| 91 | +++ (98) | +++ (85) |
| 92 | +++ (94-104) | ++++ (85-88) |
| 93 | ++++ (99) | +++ (73) |
| 94 | +++ (89) | +++ (46) |
| 95 | ++ (84) | ++ (20) |
| 96 | +++ (94) | ++++ (90) |
| 97 | +++ (99) | ++++ (93) |
| 98 | ++++ (96) | — |
| 99 | +++ (102) | — |
| 100 | ++ (95) | +++ (100) |
| 101 | ++ (92) | — |
| 102 | +++ (104) | ++ (34) |
| 103 | ++ (95) | +++ (68) |
| 104 | +++ (104-106) | +++ (43-47) |
| 105 | ++ (90) | — |
| 106 | ++ (95) | ++ (37) |
| 107 | ++ (57) | ++++ (124) |
| 108 | − (18 at 1 μM) | ++ (69) |
| 109 | +++ (101) | ++ (36) |
| 110 | ++ (88) | ++ (33) |
| 111 | ++ (94) | + (12 at 1 μM) |
| 112 | ++ (94) | ++ (26 at 1 μM) |
| 113 | ++ (89) | ++ (52) |
| 114 | ++++/+++ (94-103) | +++ (42-63) |
| 115 | +++ (91) | +++ (67) |
| 116 | +++++ (98) | ++ (50) |
| 117 | +++ (92) | ++ (70) |
| 118 | +++++ (95) | +++ (35) |
| 119 | +++++ (97) | ++ (41) |
| 120 | +++ (91) | — |
| 121 | ++++ (90) | ++ (27) |
| 122 | +++ (91) | — |
| 123 | +++ (104) | ++++ (111) |
| 124 | ++ (73) | — |
| 125 | ++ (94-100) | +++ (54-60) |
| 126 | ++ (84) | ++ (42-51) |
| 127 | +++ (95-107) | +++ (98-102) |
| 128 | +++ (99) | +++/++ (68) |
| 129 | +++/++ (91-95) | ++++/+++ (102-109) |
| 130 | ++++ (114) | +++ (64) |
| 131 | +++ (95) | — |
| 132 | +++++ (103-114) | +++ (61-64) |
| 133 | +++ (98) | ++++ (104) |
| 134 | +++++ (103-108) | ++ (30) |
| 135 | +++++/++++ | +/++ (15-22) |
| | (106-111) | |
| 136 | ++ (91-94) | ++++ (105) |
| 137 | +++ (103) | — |
| 138 | +++ (87) | +++ (81) |
| 139 | ++++ (102) | +++ (79) |
| 140 | +++++ (107) | +++ (63) |
| 141 | +++ (98-100) | ++ (46-50) |
| 142 | +++++ (101-105) | + (14.2-17) |
| 143 | ++++ (99-108) | +++ (99-107) |
| 144 | +++ (93-119) | +++++ (127-140) |
| 145 | +++ (100-104) | +++++/++++ (125) |
| 146 | +++ (86-92) | +++ (83-97) |
| 147 | +++ (87-97) | +++++ (114-128) |
| 148 | ++++ (99) | +++ (64-67) |
| 149 | +++ (86-92) | +++++ (113) |
| 150 | +++ (95-113) | +++++/++++ (118-130) |
| 151 | +++ (103-107) | ++++ (117-119) |
| 152 | ++ (85) | +++ (103) |
| 153 | +++++ (107) | ++ (75-85) |
| 154 | +++ (80) | +++ (78) |
| 155 | ++++ (102) | +++ (80) |
| 156 | +++ (95-100) | +++++ (116-118) |
| 157 | +++/++ (93-95) | ++++/+++ (103-105) |
| 158 | ++ (31-38) | ++++ (104-107) |
| 159 | ++ (87-91) | +++ (89-92) |
| 160 | +++ (85-95) | ++++ (109-113) |
| 161 | ++ (89-101) | +++/++ (58-107) |
| 162 | ++ (42-46) | ++++/+++ (103-107) |
| 163 | ++ (73) | +++++ (106) |
| 164 | ++ (68) | ++ (99-105) |
| 165 | ++++ (101-106) | +++ (91-98) |
| 166 | +++++ (109) | +++/++ (77-84) |
| 167 | ++++/+++ (94-99) | — |
| 168 | +++ (92) | — |
| 169 | +++ (96) | — |
| 170 | +++ (99) | — |
| 171 | +++ (93-98) | — |
| 172 | +++ (98-101) | ++ (63-71) |
| 173 | +++ (90-105) | ++++ (92-111) |
| 174 | ++/+ (17-73) | + (30-66) |
| 175 | ++ (87-94) | ++ (38) |
| 176 | ++ (92) | — |
| 177 | +++ (96) | — |
| 178 | +++ (97) | + (38) |
| 179 | ++++ (103) | — |
| 180 | ++ (94) | ++ (84) |
| 181 | ++ (103) | +++ (102) |
| 182 | ++++ (96) | + (12 at 1 μM) |
| 183 | ++ (95) | — |
| 184 | +++ (99) | ++ (43) |
| 185 | ++++ (97) | — |
| 186 | +++ (94) | — |
| 187 | ++ (89) | ++ (58) |
| 188 | ++ (101) | +++ (107) |
| 189 | +++ (94) | — |
| 190 | +++ (97-119) | ++++/+++ (88-119) |
| 191 | +++ (97) | ++ (68) |
| 192 | ++++ (111) | ++ (63) |
| 193 | +++ (117) | ++ (44) |
| 194 | +++ (98) | — |
| 195 | ++++/+++ (93-113) | ++++/+++ (46-87) |
| 196 | ++++/+++ (93-104) | +++/++ (41-86) |
| 197 | ++ (95) | + (20) |
| 198 | ++ (95) | ++ (81) |
| 199 | ++++ (92) | +++ (78) |
| 200 | ++ (89) | + (66) |
| 201 | +++ (99) | +++ (105) |
| 202 | +++++ | ++++ (67) |
| 203 | ++++ | +++ (44) |
| 204 | +++++ | ++ (98) |
| 208 | +++++ | +++ (59) |
| 207 | ++++ | +++ (68) |
| 205 | +++++ | +++ (61) |
| 206 | +++++ | + (18) |
| 209 | +++++ (94) | ++ (27) |
| 210 | +++++ (90) | ++ (40) |
| 211 | +++++ (91) | ++++ (50) |
| 212 | +++++ (90) | +++ (50) |
| 213 | +++++ (90) | ++++ (59) |
| 214 | +++++ (95) | +++ (45) |
| 217 | +++++ (97) | +++ (58) |
| 226 | +++ (89) | + (17) |
| 219 | ++ (89) | +++ (75) |
| 223 | +++++ (101) | ++++ (91) |
| 218 | ++++ (96) | +/++ (19-23) |
| 221 | +++ (95) | +++ (68) |
| 222 | +++ (99) | >1 mM |
| 224 | ++++ (98) | ++ (76) |
| 232 | ++++ (95) | +++ (78) |
| 233 | +++++ (96) | +++ (77) |
| 234 | ++++ (97) | +++ (76) |
| 238 | ++++ (95) | +++ (82) |
| 239 | ++++ (100) | ++ (46) |
| 240 | ++++ (97) | +++ (60) |
| 241 | +++ (100) | ++ (46) |
| 215 | +++ (103) | — |
| 243 | +++++ (103) | ++ (20) |
| 244 | +++++ (100) | +++ (50) |
| 247 | ++++ (102) | +++++ (110) |
| 248 | ++ (80) | +++ (105) |
| 249 | ++ (101) | +++ (105) |
| 220 | +++ | — |
| 254 | +++++ | +++ (80) |
| 257 | +++++ | ++ (40) |
| 260 | +++ | + |
| 264 | +++++ (100) | +++ (90) |
| 265 | +++++ (100) | ++++ (80) |
| 266 | +++++ (100) | +++ (70) |
| 267 | ++++++ (100) | ++ (40) |
| 270 | +++ (90) | +++ (90) |
| 225 | ++++ | +++ (70-80) |
| 285 | +++++ (99) | ++++ (97) |
| 291 | ++++ (100) | +++ (60) |
| 289 | +++++(100) | ++ (20) |
| 290 | +++++ (100) | ++ (10) |
| 292 | +++ (90) | ++ (50) |

### b) Glucose Tolerance Test (GTT)

### Materials:

Freestyle Lite glucometer and strips; compounds to be tested; sterilized glucose solution 200 mg/ml.

### Procedure:

Compounds were diluted in PBS from the 1 mM DMSO stocks right before injection. Body weight and baseline blood glucose level for each mouse were measured. Tails were snipped using sterile scissors; blood from tails was used to measure glucose. For 0.25 h GTT, mice were fasted for 5-6 h and injected with compound subcutaneously 15 mins before glucose injection. For 4 h GTT, mice were fasted O/N for 16 h and injected with compound subcutaneously 4 h before glucose injection. For 16 h GTT, mice were injected with compound subcutaneously 16 h before glucose injection; food was removed at the same time. For 24 h GTT, mice were injected with compound subcutaneously 24 h before glucose injection; food was removed 5-6 h before glucose injection. 20% glucose in water was prepared; the solution was sterilized by 0.2 μm-filtration. An intraperitoneal injection of glucose (2 g/kg) was given to the mouse. Continued to take blood samples from the tail. Blood glucose was measured at 10, 20, 30, 40, 60, 90, and 120 minutes after glucose injection. Between each of these time points, the mouse was returned to its cage and monitored (at least 6 replicate mice were used for each time-point). The average glucose measurements were plotted as a function of time and the area under the curve (AUC) was determined. AUC results of the GTT test are presented in Table 4, below.

TABLE 4
24 h Glucose Tolerance Test (GTT)

| | Dose | |
|---|---|---|
| | range | AUC |
| Compound # | (nmol/kg) | score |
| 1 | 2-5 | 3 |
| 2 | 2-5 | 2 |
| 5 | 2-5 | 2 |
| 16 | 2-5 | 2 |
| 18 | 2-5 | 2 |
| 32 | 2-5 | 2 |
| 41 | 2-5 | 4 |
| 59 | 2-5 | 3 |
| 64 | 2-5 | 3 |
| 65 | 2-5 | 2 |
| 66 | 2-5 | 2 |
| 68 | 2-5 | 2 |
| 69 | 2-5 | 2 |
| 77 | 5.1-15 | 2 |
| 79 | 5.1-15 | 2 |
| 81 | 2-5 | 3 |
| 83 | 2-5 | 2 |
| 91 | 2-5 | 3 |
| 96 | 5.1-15 | 4 |
| 98 | 5.1-15 | 2 |
| 114 | 2-5 | 3 |
| 195 | 2-5 | 2 |
| 233 | 2-5 | 2 |
| 234 | 2-5 | 3 |
| 264 | 2-5 | 3 |
| 266 | 2-5 | 2 |
| Liraglutide | 50 | 4 |
| Liraglutide | 200 | 2 |
| Semaglutide | 2.5 | 3 |

Note:
AUC score is based on percent compared to vehicle which is set to 100%; AUC score 1 = 0-25%, AUC score 2 = 26-50%, AUC score 3 = 51-75%, AUC score 4 = 76-100%.

### c) PathHunter β-Arrestin Assay

5,000 cells were seeded in appropriate cell culture media per well in a low volume 384-well tissue culture plate. The assay plate was incubated with cells overnight at 37° C. and 5% CO₂. The media was removed, and 5 μL of appropriate cell culture media containing 0.2% BSA was added. Agonist serial dilutions in a separate 384 well dilution plate in a 22-point series of 2× dilutions of agonist in DMSO were performed as follows. 80 μL of the highest concentration of Agonist/DMSO was added to well No. 1. 40 μL was removed from well No. 1, and added to well No. 2, followed by gentle mixing. 40 μL was removed from well No. 2 and added to well No. 3, followed by gentle mixing. This process was repeated until well No. 22. Additional serial dilutions for additional agonists were set up in a similar manner. 100 nL of each 2× agonist serial dilution was added in duplicate to the designated agonist rows of the assay plate using the Labcyte ECHO. Assay plate was incubated for 90 minutes at 37° C. and 5% CO₂. A stock of Working Detection Solution in a separate 15 ml polypropylene tube was prepared by mixing 19 parts of Cell Assay Buffer, 5 parts of Substrate Reagent 1, 1 part Substrate Reagent 2. Following agonist incubation, 3 μL of Working Detection Solution was added to all wells of the assay plate [Note: Do not pipette up and down in the vial to mix or vortex plates]. Plates were spun down briefly. Assay plate was incubated for 3 hour at room temperature in the dark for the immunocompetition reaction to occur. Samples were read on Tecan i-control plate reader using luminescence mode with 100 ms integration time. Data analysis was performed using GraphPad Prism. EC₅₀ values were only assigned to compounds with an Emax greater than 10% of positive controls (GLP-1 and liraglutide were used as positive controls for this assay). The results of the PathHunter β-arrestin assay was summarized in Table 5, below.

TABLE 5
β-arrestin activity

| Compound # | EC₅₀ | | Compound # | EC₅₀ | |
|---|---|---|---|---|---|
| 1 | >1 | μM | 114 | >1 | μM |
| 2 | >1 | μM | 116 | >1 | μM |
| 3 | >1 | μM | 118 | >1 | μM |
| 4 | 0.01-1 | μM | 119 | <0.01 | μM |
| 5 | 0.01-1 | μM | 121 | >1 | μM |
| 6 | >1 | μM | 123 | >1 | μM |
| 7 | >1 | μM | 128 | >1 | μM |
| 8 | >1 | μM | 129 | >1 | μM |
| 9 | >1 | μM | 130 | >1 | μM |
| 10 | >1 | μM | 132 | 0.01-1 | μM |
| 11 | >1 | μM | 133 | >1 | μM |
| 12 | >1 | μM | 134 | 0.01-1 | μM |
| 13 | >1 | μM | 135 | >1 | μM |
| 14 | >1 | μM | 136 | >1 | μM |
| 15 | >1 | μM | 137 | >1 | μM |
| 16 | <0.01 | μM | 138 | >1 | μM |
| 17 | >1 | μM | 139 | >1 | μM |
| 18 | <0.01 | μM | 140 | >1 | μM |
| 19 | <0.01 | μM | 141 | >1 | μM |
| 20 | >1 | μM | 142 | 0.01-1 | μM |
| 21 | >1 | μM | 143 | >1 | μM |
| 22 | >1 | μM | 144 | >1 | μM |
| 23 | >1 | μM | 145 | >1 | μM |
| 24 | >1 | μM | 146 | >1 | μM |
| 25 | >1 | μM | 147 | >1 | μM |
| 26 | >1 | μM | 148 | >1 | μM |
| 27 | >1 | μM | 149 | >1 | μM |
| 28 | >1 | μM | 150 | >1 | μM |
| 29 | >1 | μM | 151 | >1 | μM |
| 30 | >1 | μM | 152 | >1 | μM |
| 31 | >1 | μM | 153 | 0.01-1 | μM |
| 32 | >1 | μM | 154 | >1 | μM |
| 33 | >1 | μM | 155 | >1 | μM |
| 34 | >1 | μM | 156 | >1 | μM |
| 35 | >1 | μM | 157 | >1 | μM |
| 36 | >1 | μM | 158 | >1 | μM |
| 37 | >1 | μM | 159 | >1 | μM |
| 38 | >1 | μM | 160 | >1 | μM |
| 39 | >1 | μM | 161 | >1 | μM |
| 40 | >1 | μM | 162 | >1 | μM |
| 41 | >1 | μM | 163 | >1 | μM |
| 42 | >1 | μM | 164 | >1 | μM |
| 43 | >1 | μM | 165 | >1 | μM |
| 44 | >1 | μM | 166 | >1 | μM |
| 60 | >1 | μM | 167 | >1 | μM |
| 64 | >1 | μM | 170 | >1 | μM |
| 65 | >1 | μM | 171 | >1 | μM |
| 66 | >1 | μM | 172 | >1 | μM |
| 68 | >1 | μM | 173 | >1 | μM |
| 69 | >1 | μM | 176 | >1 | μM |
| 71 | >1 | μM | 177 | >1 | μM |
| 72 | >1 | μM | 178 | >1 | μM |
| 73 | >1 | μM | 179 | 0.01-1 | μM |
| 74 | >1 | μM | 180 | >1 | μM |
| 75 | >1 | μM | 181 | >1 | μM |
| 77 | >1 | μM | 182 | >1 | μM |
| 79 | >1 | μM | 183 | >1 | μM |
| 81 | >1 | μM | 184 | >1 | μM |
| 83 | >1 | μM | 185 | >1 | μM |
| 85 | >1 | μM | 186 | >1 | μM |
| 86 | >1 | μM | 187 | >1 | μM |
| 91 | >1 | μM | 188 | >1 | μM |
| 92 | >1 | μM | 189 | >1 | μM |
| 93 | >1 | μM | 190 | >1 | μM |
| 94 | >1 | μM | 191 | >1 | μM |
| 96 | >1 | μM | 192 | >1 | μM |
| 97 | >1 | μM | 193 | >1 | μM |
| 98 | >1 | μM | 194 | >1 | μM |
| 104 | >1 | μM | 195 | >1 | μM |
| 111 | >1 | μM | 196 | >1 | μM |
| GLP-1 | <0.01 | μM | liraglutide | 0.01-1 | μM |
| 202 | >1 | μM | 234 | >1 | μM |
| 203 | >1 | μM | 238 | >1 | μM |
| 204 | 0.02-1 | μM | 239 | >1 | μM |
| 208 | >1 | μM | 240 | >1 | μM |
| 207 | >1 | μM | 241 | >1 | μM |
| 205 | >1 | μM | 243 | >1 | μM |
| 206 | >1 | μM | 244 | 0.01-1 | μM |
| 209 | 0.01-1 | μM | 264 | <0.01 | μM |
| 210 | 0.01-1 | μM | 265 | <0.01 | μM |
| 211 | 0.01-1 | μM | 266 | <0.01 | μM |
| 212 | 0.01-1 | μM | 225 | >1 | μM |
| 213 | 0.01-1 | μM | 285 | >1 | μM |

| | | |
|---|---|---|
| 214 | 0.01-1 | μM |
| 217 | >1 | μM |
| 226 | >1 | μM |
| 223 | 0.01-1 | μM |
| 218 | >1 | μM |
| 232 | >1 | μM |
| 233 | 0.01-1 | μM |

### d) Conditioned Taste Aversion (CTA) in Mice

One week before start of experiment (Day-7), animals were transferred to single house double grommet cages. A 50 ml water bottle was placed, and consistently replaced when refilled in the same grommet to habituate the animal to a “water” side. After 7 days of baseline water consumption, animals began the taste preference procedure. On Day 1, the animals were weighed and deprived of water at 6 μm. On acquisition day (Day 2), mice had access to a single bottle containing 0.15% saccharin solution, on the opposite side to the “water” habituated side and allowed to consume for 3 h, from 6 pm until 9 pm. The saccharine bottles were then removed and weighed to record the 3 h consumption. The mice were assigned to form balanced groups based on saccharine consumption and animals that did not consume saccharine were removed from the study. After the groups were formed, the animals were either injected subcutaneously with test drug, vehicle (solvent) or positive aversive drug (200 nmol/kg liraglutide) based on weight from Day 1 and returned to cages with weighed water bottles. On Day 3, the water was removed and weighed at 6 pm. On Day 4, both saccharine and water bottles were weighed and returned simultaneously to respective grommet at 6 pm. On Day 5, the bottles were removed at 6 pm and weighed. Saccharine consumption was measured as a percent of the total amount of fluid consumed. The reduction of saccharin consumption during the retrieval was used as a measure of CTA strength. Liraglutide and semaglutide were used as positive controls. The results are summarized in Table 6.

TABLE 6
Conditioned taste aversion (CTA)

| | Dose | CTA |
|---|---|---|
| Compound # | (nmol/kg) | score |
| 2 | 30 | 2 |
| 5 | 30 | 1 |
| 16 | 30 | 1 |
| 32 | 30 | 2 |
| 65 | 30 | 3 |
| 66 | 50 | 2 |
| 68 | 30 | 3 |
| 69 | 30 | 3/4 |
| 83 | 30 | 2 |
| 91 | 30 | 4 |
| 96 | 30 | 4 |
| 98 | 30 | 4 |
| 114 | 50 | 2 |
| 195 | 30 | 3 |
| 202 | 30 | 4 |
| 233 | 30 | 1 |
| 234 | 30 | 1 |
| 264 | 30 | 1 |
| 266 | 30 | 1 |
| Liraglutide | 50 | 2 |
| Liraglutide | 200 | 1 |
| Semaglutide | 10 | 2 |
| Semaglutide | 20 | 1 |

Note:
Saccharine preference score is compared to vehicle (preferred saccharine solution) which is set to 100%; Score 1 = 0-25%, Score 2 = 26-50%, Score 3 = 51-75%, Score 4 = 76-100%.

A number of embodiments of the invention have been described. Nevertheless, it will be understood that various modifications may be made without departing from the spirit and scope of the invention. Accordingly, other embodiments are within the scope of the following claims.

1. A compound having formula (IAA), or a pharmaceutically acceptable salt thereof: 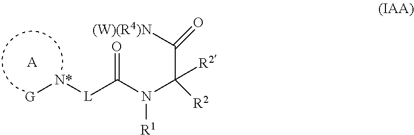
wherein:
ring A is:
(i) a saturated or unsaturated monocyclic ring that includes from 3-8 ring atoms (inclusive of G and the nitrogen atom labelled N*); or
(ii) a saturated or unsaturated bicyclic or tricyclic ring that includes from 6-14 ring atoms (inclusive of G and the nitrogen atom labelled N*), wherein:
G is C(O), S(O), or SO₂; and
the dotted, circular line connecting G and N* is a divalent group that includes from 1-6 ring atoms; wherein:
(a) from 0-2 of the divalent group's ring atoms are ring heteroatoms, which are each independently selected from the group consisting of N, N(R^{a}), O, S, and SO₂; and
(b) from 1-6 of the divalent group's ring atoms are ring carbon atoms, which are each independently selected from the group consisting of C, CH, CH₂, CR^{b}, C(R^{b})₂, and CHR^{b};
wherein:
(1) when ring A is a monocyclic ring, then each of the divalent group's 1-6 ring carbon atoms is independently selected from the group consisting of CH, CH₂, CR^{b}, C(R^{b})₂, and CHR^{b}; and
(2) when ring A is a bicyclic or tricyclic ring, then (A) or (B) applies:
(A) two or three adjacent ring carbon atoms of the divalent group are each independently selected from the group consisting of C, CH, CH₂, CR^{b}, C(R^{b})₂, and CHR^{b} and are fused to a second ring that is selected from the group consisting of:
(a) C₆₋₁₀ aryl optionally substituted with from 1-5 independently selected R^{c};
(b) heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
(c) C₃₋₁₀ cycloalkyl optionally substituted with from 1-4 independently selected R^{d};
(d) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d}; or
(B) one of the divalent group's ring carbon atoms is C and is spiro-fused to a second ring that is selected from the group consisting of:
(a) C₃₋₁₀ cycloalkyl optionally substituted with from 1-4 independently selected R^{d};
(b) heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d};
L is:
(i) —(CH₂)ₘ—X¹—(CH₂)ₙ—X²—(CH₂)ₚ— (formula VIII), wherein m is from 1-6; n is from 0-2; and p is from 0-8;
(ii) —C(O)—(CH₂)ₙ—X³—(CH₂)ₚ— (formula IX), wherein n is from 0-2; and p is from 0-8;
(iii) —(CH₂)_{q}—, wherein q is from 1-10;
(iv) —C(O)—; or
(v) —(CH₂)ₘ—X¹—(CH₂)ₚ— (formula VIII), wherein m is from 1-6; and p is from 0-8;
X¹ is —C(O)—; —N(R′)C(O)—; —C(O)N(R′)—; or —N(R′)C(O)NR′)—; wherein each occurrence of R′ is independently selected from the group consisting of: H and C₁₋₄ alkyl;
X² is:
(i) —O—;
(ii) —S—;
(iii) —S(O)ₜ—, wherein t is 1 or 2;
(iv) —C(Q¹)(Q²)-, wherein each of Q¹ and Q² is independently selected from the group consisting of H and C₁₋₄ alkyl; or Q¹ and Q², together with the carbon atom to which each is attached forms C₃₋₁₀ cycloalkyl that is optionally substituted with from 1-4 independently selected R^{d} and optionally fused to phenyl;
(v) C₆₋₁₀ arylene optionally substituted with from 1-5 independently selected R^{c};
(vi) heteroarylene including from 5-10 ring atoms, wherein from 1-4 ring atoms are each independently selected from the group consisting of N, N(R^{a}), O, and S; and which is optionally substituted with from 1-5 independently selected R^{c};
(vii) C₃₋₁₀ cycloalkylene optionally substituted with from 1-4 independently selected R^{d};
(viii) heterocycloalkylene including from 3-10 ring atoms, wherein from 1-3 ring atoms are each independently selected from the group consisting of N(R^{a}), O, and S; and
which is optionally substituted with from 1-5 independently selected R^{d};
(ix) C₂-C₄ alkenylene optionally substituted with from 1-2 R^{e}; or
(x) C₂-C₄ alkynylene optionally substituted with from 1-2 R^{e};
X³ is: —O—; —S—; or —S(O)ₜ—, wherein tis 1 or 2;
each of R¹, R², and R⁴ is independently selected from the group consisting of H and unsubstituted C₁₋₃ alkyl;
R^{2′} is as defined according to (AA) or (BB) below:
(AA)
R^{2′} is 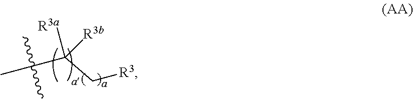
wherein:
R³ is —C(O)OH, —C(O)OR³¹, —CH(C(O)OH)₂ or a carboxylic acid isostere (e.g., amide or tetrazolyl);
a is 0-5;
a′ is 0 or 1; and
each of R^{3a} and R^{3b} is independently H or C₁₋₃ alkyl;
(BB)
R^{2′} and R⁴ taken together with the atoms connecting them form a ring including from 5-8 ring atoms, wherein from 3-7 are ring carbon atoms each substituted with from 1-2 substituents independently selected from H, halo, hydroxy, oxo, and C₁₋₃ alkyl; and from 0-1 is a heteroatom (in addition to the N attached to R¹) selected from O, —NH, —N(C₁₋₃ alkyl), and S (e.g., R⁴ and R³, taken together with the atoms to which each is attached form a ring including 5-8 ring atoms);
R³¹ is:
(i) C₁₋₆ alkyl optionally substituted with from 1-2 independently selected R^{e};
(ii) —(C₀₋₃ alkylene)-C₃₋₁₀ cycloalkyl, wherein the cycloalkyl is optionally substituted with from 1-4 independently selected R⁴;
(iii) —(C₀₋₃ alkylene)-heterocyclyl including from 3-10 ring atoms, wherein from 1-3 ring atoms are each independently selected from the group consisting of N(R^{a}), O, and S, wherein the heterocyclyl is optionally substituted with from 1-4 independently selected R^{d},
(iv) —(C₀₋₃ alkylene)-C₆₋₁₀ aryl, wherein the aryl is optionally substituted with from 1-5 independently selected R^{c}; or
(v) —(C₀₋₃ alkylene)-heteroaryl including from 5-10 ring atoms, wherein from 1-4 ring atoms are each independently selected from the group consisting of N, N(R^{a}), O, and S, wherein the heteroaryl is optionally substituted with from 1-3 independently selected R^{c};
each occurrence of R^{a} is independently selected from the group consisting of: H; C₁₋₄ alkyl; C₃₋₆ cycloalkyl; —C(═O)(C₁₋₆ alkyl); —C(═O)O(C₁₋₆ alkyl); and —S(O)₁₋₂(C₁₋₆ alkyl);
each occurrence of R^{b} is independently selected from the group consisting of: R³¹; C₁₋₄ haloalkyl; —OH; oxo; —F; —N(R^{a})(R″); C₁₋₄ alkoxy; C₁₋₄ haloalkoxy; —C(═O)(C₁₋₄ alkyl); —C(═O)O(C₁₋₆ alkyl); —C(═O)OH, —C(═O)N(R′″)(R″″), —S(O)₁₋₂(C₁₋₆ alkyl); and cyano;
each occurrence of R^{c} is independently selected from the group consisting of: C₁₋₆ alkyl optionally substituted with from 1-2 independently selected R^{e}; C₃₋₆ cycloalkyl; C₁₋₄ haloalkyl; —OH; -halo; —NO₂; N₃; —N(R^{a})(R″); C₁₋₄ alkoxy; C₁₋₄ thioalkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R′″)(R″′), —S(O)₁₋₂(C₁₋₆ alkyl); and cyano;
each occurrence of R^{d} is independently selected from the group consisting of: C₁₋₆ alkyl optionally substituted with from 1-2 independently selected R^{e}; C₁₋₄ haloalkyl; —OH; —F; Cl; —N(R^{a})(R″); oxo; C₁₋₄ alkoxy; C₁₋₄ haloalkoxy; —OC(═O)(C₁₋₆ alkyl); —C(═O)(C₁₋₆ alkyl); —C(═O)O(C₁₋₆ alkyl); —C(═O)OH, —C(═O)N(R″′)(R″″), —S(O)₁₋₂(C₁₋₆ alkyl); and cyano;
each occurrence of R^{e} is independently selected from the group consisting of: —OH; —N(R^{a})(R″); C₁₋₄ alkoxy; C₁₋₄ haloalkoxy; —OC(═O)(C₁₋₆ alkyl); —C(═O)O(C₁₋₆ alkyl); —C(═O)OH, —C(═O)N(R″′)(R″″), —S(O)₁₋₂(C₁₋₆ alkyl); and cyano;
each occurrence of R″, R′″, and R″″ is independently selected from the group consisting of: H and C₁₋₆ alkyl; and
W is a peptide having the formula W′—R⁵, wherein W′ is a sequence of from 5-60 amino acids, and R⁵ is a C-terminal amino acid, amino acid ester, or amino acid amide that is optionally substituted with from 1-2 modifying groups (e.g., 1-2 groups selected from an acyl group and a PEG group); and
provided that one or more of the compound provisions delineated herein apply.

2-218. (canceled)
